SEQUENCE LISTING (80 sequences)

SEQ ID NO: 1 - DNA, 1196 bp, bovine cDNA
aggaaaaagtaagaacaagggaaaagattgtattgattttaaaaccatgcaaaaactgcaaatctctgtttatatttacctatttatgctgattgttgctggcccagtggatctgaatgagaacagcgagcagaaggaaaatgtgcaaaaagaggggctgtgtaatgcatgtttgtggagggaaaacactacatcctcaagactagaagccataaaaatccaaatcctcagtaaacttcgcctggaaacagctcctaacatcagcaaagatgctatcagacaacttttgcccaaggctcctccactcctggaactgattgatcagttcgatgtccagagagatgccagcagtgacggctccttggaagacgatgactaccacgccaggacggaaacggtcattaccatgcccacggagtctgatcttctaacgcaagtggaaggaaaacccaaatgttgcttctttaaatttagctctaagatacaatacaataaactagtaaaggcccaactgtggatatatctgaggcctgtcaagactcctgcgacagtgtttgtgcaaatcctgagactcatcaaacccatgaaagacggtacaaggtatactggaatccgatctctgaaacttgacatgaacccaggcactggtatttggcagagcattgatgtgaagacagtgttgcagaactggctcaaacaacctgaatccaacttaggcattgaaatcaaagctttagatgagaatggccatgatcttgctgtaaccttcccagaaccaggagaagatggactgactccttttttagaagtcaaggtaacagacacaccaaaaagatctaggagagattttgggcttgattgtgatgaacactccacagaatctcgatgctgtcgttaccctctaactgtggattttgaagcttttggatgggattggattattgcacctaaaagatataaggccaattactgctctggagaatgtgaatttgtatttttgcaaaagtatcctcatacccatcttgtgcaccaagcaaaccccagaggttcagccggcccctgctgtactcctacaaagatgtctccaattaatatgctatattttaatggcgaaggacaaataatatacgggaagattccagccatggtagtagatcgctgtgggtgttcatgagtctatatttgggttcataagc

SEQ ID NO: 2 - protein, 375 aa, bovine
MQKLQISVYIYLFMLIVAGPVDLNENSEQKENVEKEGLCNACLWRENTTSSRLEAIKIQILSKLRLETAPNISKDAIRQLLPKAPPLLELIDQFDVQRDASSDGSLEDDDYHARTETVITMPTESDLLTQVEGKPKCCFFKFSSKIQYNKLVKAQLWIYLRPVKTPATVFVQILRLIKPMKDGTRYTGIRSLKLDMNPGTGIWQSIDVKTVLQNWLKQPESNLGIEIKALDENGHDLAVTFPEPGEDGLTPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGEGQIIYGKIPAMVVDRCGCS

SEQ ID NO: 3 - DNA, 1240 bp, bovine cDNA
aggaaaaagtaagaacaagggaaaagattgtattgattttaaaaccatgcaaaaactgcaaatctctgtttatatttacctatttatgctcattgttgctggcccagtggatctgaatgagaacagcgagcagaaggaaaatgtggaaaaagaggggctgtgtaatgcatgtttgtggagggaaaacactacatcctcaagactagaagccataaaaatccaaatcctcagtaaacttcgcctggaaacagctcctaacatcagcaaagatgctatcagacaacttttgcccaaggctcctccactcctggaactgattgatcagttcgatgtccagagagatgccagcagtgacggctccttggaagacgatgactaccacgccaggacggaaacggtcattaccatgcccacggagtctgatcttctaacgcaagtggaaggaaaacccaaatgttgcttctttaaatttagctctaagatacaatacaataaactagtaaaggcccaactgtggatatatctgaggcctgtcaagactcctgcgacagtgtttgtgcaaatcctcagactcatcaaacccatgaaagacggtacaaggtatactggaatccgatctctgaaacttgacatgaacccaggcactggtatttggcagagcattgatgtgaagacagtgttgcagaactggctcaaacaacctgaatccaacttaggcattgaaatcaaagctttagatgagaatggccatgatcttgctgtaaccttcccagaaccaggagaagatggactgactccttttttagaagtcaaggtaacagacacaccaaaaagatctaggagagattttgggcttgattgtgacagaatctcgatgctgtcgttaccctctaactgtggattttgaagcttttggatgggattggattattgcacctaaaagatataaggccaattactgctctggagaatgtgaatttgtatttttgcaaaagtatcctcatacccatcttgtgcaccaagcaaaccccagaggttcagccggcccctgctgtactcctacaaagatgtctccaattaatatgctatattttaatggcgaaggacaaataatatacgggaagattccagccatggtagtaaatcgctgtgggtgttcatgaggtctatatttggttcatagcttcctcaaacatggaaggtcttcccctcaacaattttgaaactgttgaaattatgt

SEQ ID NO: 4 - protein, 286 aa, bovine
MQKLQISVYIYLFMLIVAGPVDLNENSEQKENVEKEGLCNACLWRENTTSSRLEAIKIQILSKLRLETAPNISKDAIRQLLPKAPPLLELIDQFDVQRDASSDGSLEDDDYHARTETVITMPTESDLLTQVEGKPKCCFFKFSSKIQYNKLVKAQLWIYLRPVKTPATVFVQILRLIKPMKDGTRYTGIRSLKLDMNPGTGIWQSIDVKTVLQNWLKQPESNLGIEIKALDENGHDLAVTFPEPGEDGLTPFLEVKVTDTPKRSRRDFGLDCDRISMLSLPSNCGF

SEQ ID NO: 5 - DNA, 2676 bp, murine cDNA
gtctctcggacggtacatgcactaatatttcacttggcattactcaaaagcaaaaagaagaaataagaacaagcgaaaaaaaaagattgtgctgatttttaaaatgatgcaaaaactgcaaatgtatgtttatatttacctcttcatgctgattgctgctggcccagtggatctaaatgagggcagtgagagagaagaaaatgtggaaaaagaggggctgtgtaatgcatgtgcgtggagacaaaacacgaggtactccagaatagaagccataaaaattcaaatcctcagtaagctgcgcctggaaacagctcctaacatcagcaaagatgctataagacaacttctgccaagagcgcctccactccgggaactgatcgatcagtacgacgtccagagggatgacagcagtgatggctctttggaagatgacgattatcacgctaccacggaaacaatcattaccatgcctacagagtctgactttctaatgcaagcggatggcaagcccaaatgttgcttttttaaatttagctctaaaatacagtacaacaaagtagtaaaagcccaactgtggatatatctcagacccgtcaagactcctacaacagtgtttgtgcaaatcctgagactcatcaaacccatgaaagacggtacaaggtatactggaatccgatctctgaaacttgacatgagcccaggcactggtatttggcagagtattgatgtgaagacagtgttgcaaaattggctcaaacagcctgaatccaacttaggcattgaaatcaaagctttggatgagaatggccatgatcttgctgtaaccttcccaggaccaggagaagatgggctgaatccctttttagaagtcaaggtgacagacacacccaagaggtcccggagagactttgggcttgactgcgatgagcactccacggaatcccggtgctgccgctaccccctcacggtcgattttgaagcctttggatgggactggattatcgcacccaaaagatataaggccaattactgctcaggagagtgtgaatttgtgtttttacaaaaatatccgcatactcatcttgtgcaccaagcaaaccccagaggctcagcaggcccttgctgcactccgacaaaaatgtctcccattaatatgctatattttaatggcaaagaacaaataatatatggcaaaattccagccatggtagtagaccgctgtgggtgctcatgagctttgcattaggttagaaacttcccaagtcatggaaggtcttcccctcaatttcgaaactgtgaattcaagcaccacaggctgtaggccttgagtatgctctactaacgtaagcacaagctacagtgtatgaactaaaagagagaatagatgcaatggttggcattcaaccaccaaaataaaccatactataggatgttgtatgatttccagagtttttgaaatagatggagatcaaattacatttatgtccatatatgtatattacaactacaatctaggcaaggaagtgagagcacatcttgtggtctgctgagttaggagggtatgattaaaaggtaaagtcttatttcctaacagtttcacttaatatttacagaacaatctatatgtagcctttgtaaagtgtaggattgttatcatttaaaaacatcatgtacacttatatttgtattgtatacttggtaagataaaattccacaaagtaggaatggggcctcacatacacattgccattcctattataattggacaatccaccacggtgctaatgcagtgctcaatggctcctactggacctctcgatagaacactctacaaagtacgagtctctctctcccttccaggtgcatctccacacacacagcactaagtgttcaatgcattttctttaaggaaagaagaatctttttttctagaggtcaactttcagtcaactctagcacagcgggagtgactgctgcatcttaaaaggcagccaaacagtattcattttttaatctaaatttcaaaatcactgtctgcctttatcacatggcaattttgtggtaaaataatggaaatgactggttctatcaatattgtataaaagactctgaaacaattacatttatataatatgtatacaatattgttttgtaaataagtgtctccttttatatttactttggtatatttttacactaatgaaatttcaaatcattaaagtacaaagacatgtcatgtatcacaaaaaaggtgactgcttctatttcagagtgaattagcagattcaatagtggtcttaaaactctgtatgttaagattagaaggttatattacaatcaatttatgtattttttacattatcaacttatggtttcatggtggctgtatctatgaatgtggctcccagtcaaatttcaatgccccaccattttaaaaattacaagcattactaaacataccaacatgtatctaaagaaatacaaatatggtatctcaataacagctacttttttattttataatttgacaatgaatacatttcttttatttacttcagttttataaattggaactttgtttatcaaatgtattgtactcatagctaaatgaaattatttcttacataaaaatgtgtagaaactataaattaaagtgttttcacatttttgaaaggc

SEQ ID NO: 6 - protein, 376 aa, murine
MMQKLQMYVYIYLFMLIAAGPVDLNEGSEREENVEKEGLCNACAWRQNTRYSRIEAIKIQILSKLRLETAPNISKDAIRQLLPRAPPLRELIDQYDVQRDDSSDGSLEDDDYHATTETIITMPTESDFLMQADGKPKCCFFKFSSKIQYNKVVKAQLWIYLRPVKTPTTVFVQILRLIKPMKDGTRYTGIRSLKLDMSPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 7 - DNA, 2215 bp, human cDNA
tgattttctaatgcaagtggatggaaaacccaaatgttgcttctttaaatttagctctaaaatacaatacaataaagtagtaaaggcccaactatccatatatttgagacccgtcgagactcctacaacagtgtttgtgcaaatcctgagactcatcaaacctatgaaagacggtacaaggtatctggaatccgatctctgaaacttgacatgaacccaggcactggtatttgggcagacattgatgtgaagacactgttgcaaaattggctcaaacaacctgaatccaacttaggcattgaaataaaagctttacatgagaatggtcatgatcttgctgtaaccttcccaggaccaggaagaagatgggctgaatcccttttttaagaaggtcaaggtaacagacacaccaaaaagattccagaagggattttgggtcttgactggtgatgagcactcaacagaatcacgatcctgtcgttaccccctaactggtggattttgaagcctttgggatgggattggatatcgctcctaaaagaaagatataaggccaattactgctctggagagtgtgaatttgtatttttacaaaaatatcctcatactcatctggtacaccaagcaaaccccagaggttcagcaggcccttgctgtactcccacaaagatgtctccaattaatatgctatattttaatggcaaagaacaaataatatatgggaaaattccagcgatggtagtagaccgctgtgggtgctcagcgatggtagtagaccgctgtgggtgctcttttcaagctgtgaaattaagtaccacaggctataggcctagagtatgctacagtcacttaagcataagctacagtatgtaaactaaaagggggaaggggaatatatgcaatggttggcatttaaccatccaaacaaatcataccagaaagttttatgatttccagagttttttgaggcgagaaaggaggagtcaaaatttcattcttatggttatatattacaacatcggcgaggaaatgaaagcgattctccttgagttctgatgaattaaaggagtatgctttaaagtctatttctttaaagttttgtttaatatttacagaaaaatccacatacagtattggtaaaatgcaggattgttatataccatcattcgaatcaatttcggcacaggttaaacacttgaatttatattgtatggtagtatacttggtaagataaaattccacaaaaatagggatggtgcagcatatgcaatttccattcctattataattgacacagtacattaacaatccatgccaacggtgctaatacgataggctgaatggattttctttaatgtaagaagaatcatttttctagaggttggctttcaattctgtagcatacttggagaaactgcattatcttaaaaggcagtcaaatggtgtttgtttttatcaaaatgtcaaaataacatacttggagaagtatgtaattttgtctttggaaaattacaacactgcctttgcaacactgcagtttttatggtaaaataatagaaatgatcgactctatcaatattgtataaaaagactgaaacaatgcatttatataatatgtatacaatattgttttgtaaataagtgtctccttttttatttactttggtatatttttacactaaggacatttcaaattaagtactaaggcacaaagacatgtcatgcatcacagaaaagcaactacttatatttcagagcaaattagcagattaaatagtggtcttaaaactccatatgttaatgattagatggttatattacaatcattttatatttttttacatgattaacattcacttatggattcatgatggctgtataaagtgaatttgaaatttcaatggtttactgtcattgtgttaaatctcaacgttccattattttaatacttgcaaaaacattactaagtataccaaaataattgactctattatctgaaatgaagaataaactgatgctatctcaacaataactgttacttttattttataatttgataatgaatatatttctgcatttatttacttctgttttgtaaattgggattttgttaatcaaatttattgtactatgactaaatgaaattatttcttacatctaatttgtagaaacagtataagttatattaaagtgttttcacatttttttgaaagac

SEQ ID NO: 8 - protein, 375 aa, human
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 9 - DNA, 22 bp, Artificial
  primer sequence
ggcccaactatggatatatttg

SEQ ID NO: 10 - DNA, 21 bp, Artificial
  primer sequence
ggtcctgggaaggttacagca

SEQ ID NO: 11 - DNA, 11 bp, bovine
atgaacactcc

SEQ ID NO: 12 - DNA, 26 bp, Artificial
  primer sequence
cagcaaagtccttaatggtaacaagc

SEQ ID NO: 13 - DNA, 23 bp, Artificial
  primer sequence
gggtcactgaagaaaacgtcctg

SEQ ID NO: 14 - DNA, 23 bp, Artificial
  primer sequence
ccccatattatggagatgaaccg

SEQ ID NO: 15 - DNA, 23 bp, Artificial
  primer sequence
agttcaggatggcagaatttcag

SEQ ID NO: 16 - DNA, 23 bp, Artificial
  primer sequence
gcaaactgggyggragcaagacc

SEQ ID NO: 17 - DNA, 24 bp, Artificial
  primer sequence
ttsttcctgggcttttattgagac

SEQ ID NO: 18 - DNA, 25 bp, Artificial
  primer sequence
aagccwgatttctgcttyttggaag

SEQ ID NO: 19 - DNA, 25 bp, Artificial
  primer sequence
tgccmaggcahccrccrtacttgaa

SEQ ID NO: 20 - DNA, 19 bp, Artificial
  primer sequence
ggtcgtcctacaccagaag

SEQ ID NO: 21 - DNA, 22 bp, Artificial
  primer sequence
ggttgacattgtcaagaacaag

SEQ ID NO: 22 - DNA, 24 bp, Artificial
  primer sequence
tctcmaaagtcgtctgtgacaatc

SEQ ID NO: 23 - DNA, 23 bp, Artificial
  primer sequence
tgytcrttttctttcagagttgc

SEQ ID NO: 24 - DNA, 23 bp, Artificial
  primer sequence
rctggtcctcttcacctcagaac

SEQ ID NO: 25 - DNA, 23 bp, Artificial
  primer sequence
acattgtcvgttccaaagccaag

SEQ ID NO: 26 - DNA, 21 bp, Artificial
  primer sequence
aggtycgggtgacdgtgctkc

SEQ ID NO: 27 - DNA, 23 bp, Artificial
  primer sequence
tggrtacatgagytccaccttgc

SEQ ID NO: 28 - DNA, 23 bp, Artificial
  primer sequence
agctgcargtatwcctacaayct

SEQ ID NO: 29 - DNA, 23 bp, Artificial
  primer sequence
gtyccrttgctcytctcrttgyc

SEQ ID NO: 30 - DNA, 25 bp, Artificial
  primer sequence
aactgtatattgagagcctaccatg

SEQ ID NO: 31 - DNA, 24 bp, Artificial
  primer sequence
cacaccttagcgactaaaccacca

SEQ ID NO: 32 - DNA, 25 bp, Artificial
  primer sequence
ctacctaacagaatgattttgtaag

SEQ ID NO: 33 - DNA, 25 bp, Artificial
  primer sequence
agtgttcttgcctagagaatcccag

SEQ ID NO: 34 - DNA, 25 bp, Artificial
  primer sequence
acattctctcaccaatatgacatac

SEQ ID NO: 35 - DNA, 25 bp, Artificial
  primer sequence
taagtcaccattacatccttagaac

SEQ ID NO: 36 - DNA, 25 bp, Artificial
  primer sequence
gctgtaagaatcttcattaagcact

SEQ ID NO: 37 - DNA, 25 bp, Artificial
  primer sequence
cctgatacatgctaaggttaaaaac

SEQ ID NO: 38 - DNA, 25 bp, Artificial
  primer sequence
aggcatacatctggagagaaacatg

SEQ ID NO: 39 - DNA, 25 bp, Artificial
  primer sequence
cagaggagcctagcaggctaccgtc

SEQ ID NO: 40 - DNA, 25 bp, Artificial
  primer sequence
cagcaggtctgttgaagtgtatcag

SEQ ID NO: 41 - DNA, 25 bp, Artificial
  primer sequence
agtggtagcattcacaggtagccag

SEQ ID NO: 42 - DNA, 20 bp, Artificial
  primer sequence
cagtccatggcaccataaag

SEQ ID NO: 43 - DNA, 22 bp, Artificial
  primer sequence
tccgttagtactggctaattgc

SEQ ID NO: 44 - DNA, 20 bp, Artificial
  primer sequence
ctgaattggctccaaaggcc

SEQ ID NO: 45 - DNA, 20 bp, Artificial
  primer sequence
aaacagaagtccagggctgc

SEQ ID NO: 46 - DNA, 21 bp, Artificial
  primer sequence
tcagtctccaggagagaaaac

SEQ ID NO: 47 - DNA, 20 bp, Artificial
  primer sequence
ctctgccctggggatgattg

SEQ ID NO: 48 - DNA, 28 bp, Artificial
  primer sequence
aatgtatgtttatatttacctgttcatg

SEQ ID NO: 49 - DNA, 25 bp, Artificial
  primer sequence
acagtgtttgtgcaaatcctgagac

SEQ ID NO: 50 - DNA, 25 bp, Artificial
  primer sequence
caatgcctaagttggattcaggttg

SEQ ID NO: 51 - DNA, 25 bp, Artificial
  primer sequence
cttgctgtaaccttcccaggaccag

SEQ ID NO: 52 - DNA, 23 bp, Artificial
  primer sequence
tcccatccaaaggcttcaaaatc

SEQ ID NO: 53 - DNA, 25 bp, Artificial
  primer sequence
atactcwaggcctayagcctgtggt

SEQ ID NO: 54 - DNA, 5790 bp, bovine
gcggccgcccgggcaggtatcgaaagtttcacatataaagatgaataagatctaagtgtatatgttattgttaataaagtttttaatttttcgaatgtcacatacagcctttattattcatagatttattccttttaagaagtagtcaaatgaatcagctcacccttgactgtaacaaaatactgtttggtgacttgtgacagacagggttttaacctctgacagcgagattcattgtggagcaagagccaatcacagatcccgacgacacttgtctcatcaaagttggaatataaaaagccacttggaatacagtataaaagattcactggtgtggcaagttgtctctagactgggcaggcattaacgtttggcttggcgttactcaaaagcaaaagaaaagtaaaaggaagaagtaagaacaagggaaaagattgtattgattttaaaaccatgcaaaaactgcaaatctctgtttatatttacctatttatgctgattgttgctggcccagtggatctgaatgagaacagcgagcagaaggaaaatgtggaaaaagaggggctgtgtaatgcatgtttgtggagggaaaacactacatcctcaagactagaagccataaaaatccaaatcctcagtaaacttcgcctggaaacagctcctaacatcagcaaagatgctatcagacaacttttgcccaaggctcctccactcctggaactgattgatcagttcgatgtccagagagatgccagcagtgacggctccttggaagacgatgactaccacgccaggacggaaacggtcattaccatgcccacggagtgtgagtagtcctgctggtgcaaagcaacgactctgctgactgctgttctagtgttcatgaaaaaccgatctattttcaggctcttttaacaagctgctggcttgtatgtaaggaggaggggaaagagcttttttcaagatttcatgagaaatagaccaatgagactgaaagctgctactttatttgtttccttagagagctaaaaagctaaaaatcaaaaatgaaatgcttgcatagcattcatgttatatagtttagtatgacaactataacatgtttatgttttcacagcttaatgctaccaaggtaaaggattgggaaacagtatcagcaatgtgaaaaatttacatcaaatttcctaattgcatttggttgcctgaaatatgcatttataataacaggttttttttttttcattaataaaagagaaaggaagaaatctgtagaggttgaagcctatctgggcatttgctgaacacttagaatgacttctgttattcaaaactatttctcatagggtttttatggtcttcacagagtatctaattttgaaagctattagagtggaaaggataaaagaatattcttaataaacttaatgtattagtaagagcaataaggaagtaaacacagcatagtgaaaaatcatgagctaatcagcagaaaattctaagaaataaacattttaattacaaagttccacttataccctgaccatggtactattgttgagagtaccttgtctgcacatatctaggaggcacatgcttaataaccttctaaaatattattgtattcctcataggagggagaactattacctatatgtagtacctatgttgtttctgaaagataatatgtttcatgtatttctgttgcagtcacttcaaacctatactcaaggaaagggagacaggcatctcaacagagaaggcatgaccagaaagagttttgtgccatgtgtctgcgatcttgctttatacagggctctacccactttaaactggactcaaaacagtttcaaaatactgctttttcttattaagtaactagtttataaggcaacaaataaatttcctttaagactgtgctatcagataatcctggaatagatttgccttacttataaacaatcttgagaaaacaaaaaggcaagaaattgctaagtgcttctgcttacaatgacagcctggccctaaagacaatgttttctaagttttgaaacagcttgaatacaacatctaagttttggtgctaattacctgctagtttttttatttttttcctttaaaaggctgtcccagcgtcctaacataacagatgcactatattttctgctaattcccgaggctcagttagttgctcactgtgtcttgtccccaggtaattcaggcctgggggaagggttccttcctccagactgattggtacagctgctcagtaagtgtaactactcagattcccaaagaattctaagtggatgttcttccacagtgtctcttgttctctctaatcatcatcattttaaaatttcatccacttttcattccttaatagaattttccttagtccacagttctctggaaaggaagtaggcttctcataacagctgaaaaaacatatacctaaaagattctgaaaagctgtaataactgttatacttgatattttgctgttatgaatgaaatgctacatatttttccattttaaaagactaaatatgcacacattattccaattaaaaaatgttcatagattgatatggaggtgttcgttcatttttcataaaaatgatcttagtaactttttttcttattcatttatagctgatcttctaacgcaagtggaaggaaaacccaaatgttgcttctttaaatttagctctaagatacaatacaataaactagtaaaggcccaactgtggatatatctgaggcctgtcaagactcctgcgacagtgtttgtgcaaatcctgagactcatcaaacccatgaaagacggtacaaggtatactggaatccgatctctgaaacttgacatgaacccaggcactggtatttggcagagcattgatgtgaagacagtgttgcagaactggctcaaacaacctgaatccaacttaggcattgaaatcaaagctttagatgagaatggccatgatcttgctgtaaccttcccagaaccaggagaagatggactggtaagtgattactgaaaataacatgctaaaaaccttgttatgtgtttattcataatgtgaatgaatagtagtgaaaaataactaccagtttcctgtgcttataagccagacaaaggcaccttaccccagtggtagccctgtactcaataaaagtaggtgtcccatttcacatcctatgaaacactctcttgatactttgactttgcatgaggatttaaaagaaaaaaagttataccatggtccttaagtttttagggaattctttggaattgagaatgaaatataaaatgctttccgttgatgtgctacatgattatataaataaaaacatgaagtcttcacagtggattctagtactcacccaacaacacattttttcccccagaagagtgaccaatttgttaaaattcttttgcttaataaggcagaaaaatgaactctacaagttataattaaaataaaatgcttttacttatagaaattaactagatatatgttcaggtttatatactattaaatatactatatttaagatctctcatgataaatatgttccttgttttatagactattgatgcactgatgtatatgtggattactttgtgaattacccctggtaaaattaaaaatttcaggctagttaacttgtactacttagctattttctgaactgtcttactgttctttaacaggagttaacttaggtaatgtcaactaatttaatataaagtcaaacagaaaataatgccttatatattataaaaattaataaaaaaccattttaaaatctagtataagtttagagctactcactcttctggcttatctatgcttgtatttacttctgttttcaaaaaattttttaatgtgaccataccttttatttccagttattgatataatttacaacaaaagattatacttgcaagctttatagtttttaaatggtcttatttgtagtgaatatcatatctaaatgatatctaaatgtaaagtaaatcatacctaaatgaaaacatattctttaagtcattataaaattttccaggtgatcaatttttctttaaatatactacataaaatgttattgactcccaaaatgatgttattttgtataatcttaaataccaataattaccaggtctattttggttttagtgtaggataaaaaagaatgtgttcttttttctaggtagcattttaatgatcaaagttggtgacgtgacagaggtcttaagtattattaaacagatgattaataagatgtattcctcagacttttccatataaaaggaaaaatgtctcaaattcatgaaaagattggtacaggaggaggattagcaaattgtagtttaaatatctgaatggaaacactttttagtgaaagaataaagggaatatcattgtatcttcttctgagtctgtgcctctctctcttggagttagtctttccaaccctatatacttaccactatcttcatccctctaccttcctttttcccattacatctgtgcagtactgggtggcaactattgtgtttcggtgttaatatccaagtttccctgaataagaccaagtgaatggaggatgaatgagtatacctatccctccaggggtcatcagacatatttagccaccatatttaatcaataagcaggaagacataagctagccttgtccttcttctttcctccctgctcctttctcttctcttccccctctccctttactgtcatccatcagtattttcagagcatctattatgtgtcaggcattcagatactcaaacggaggaaaacaagaataaacaagacaaagatctgaccacaggggaatccctatggctactgtagacttttgagccataaaggaagaatcaagcctagtgtaaatgaaaattccttaatgctgtgccttttaaaaagaaatgtgacataagcaaaatgattagtttctttctttaataatgagtccttgaggtaggagagtgttttgggatctattattaactcttctttcctttccatacagactccttttttagaagtcaaggtaacagacacaccaaaaagatctaggagagattttgggcttgattgtgatgaacactccacagaatctcgatgctgtcgttaccctctaactgtggattttgaagcttttggatgggattggattattgcacctaaaagatataaggccaattactgctctggagaatgtgaatttgtatttttgcaaaagtatcctcatacccatcttgtgcaccaagcaaaccccagaggttcagccggcccctgctgtactcctacaaagatgtctccaattaatatgctatattttaatggcgaaggacaaataatatacgggaagattccagccatggtagtagatcgctgtgggtgttcatgaggtctatatttggttcatagcttcctcaaacatggaaggtcttcccctcaacaattttgaaactgtgaaattatgtaccacaggctataagcctagagtatgctacagtcacttaagcacaagctacagtatatgagctaaaaagagagaatatatgcaatggttggcatttaaccatccaaacaaatcgtataataaaaagttttatgatttccagagtttttgaactaggagatcaaattccatttatgttgaaatatattacaacacatgcaggtgaatgaaagcaattctccttgtcttctggtgaattaaaggagtatgctttaaaatctatttctctacagtttc

SEQ ID NO: 55 - DNA, 10 bp, bovine
aagcatacaa

SEQ ID NO: 56 - DNA, 18 bp, bovine
attgatcagttagatgtc

SEQ ID NO: 57 - protein, 6 aa, bovine
IDQLDV

SEQ ID NO: 58 - DNA, 21 bp, bovine
tgcttctaagcatacaatagc

SEQ ID NO: 59 - DNA, 17 bp, bovine
gattagtttctttcttt

SEQ ID NO: 60 - DNA, 13 bp, bovine
gtttgtttttcat

SEQ ID NO: 61 - DNA, 13 bp, bovine
gttcatttttcat

SEQ ID NO: 62 - DNA, 18 bp, bovine
ggtatttggtagagcatt

SEQ ID NO: 63 - DNA, 14 bp, bovine
ttttttcttattca

SEQ ID NO: 64 - DNA, 18 bp, bovine
cccaaatgttgtttcttt

SEQ ID NO: 65 - DNA, 18 bp, bovine
ttaggcatttaaatcaaa

SEQ ID NO: 66 - DNA, 18 bp, bovine
tctggagaatatgaattt

SEQ ID NO: 67 - protein, 6 aa, bovine
SGEYEF

SEQ ID NO: 68 - DNA, 21 bp, Artificial
  primer sequence
tctaggagagattttgggctt

SEQ ID NO: 69 - DNA, 23 bp, Artificial
  primer sequence
gatgggtatgaggatacttttgc

SEQ ID NO: 70 - DNA, 27 bp, Artificial
  primer sequence
ctagtttattgtattgtatcttagagc

SEQ ID NO: 71 - DNA, 22 bp, Artificial
  primer sequence
agactcctacaacagtgtttgt

SEQ ID NO: 72 - DNA, 25 bp, Artificial
  primer sequence
atactcwaggcctayagcctgtggt

SEQ ID NO: 73 - DNA, 27 bp, Artificial
  primer sequence
ccctcctccttacatacaagccagcag

SEQ ID NO: 74 - DNA, 26 bp, Artificial
  primer sequence
ataagcacaggaaactggtagttatt

SEQ ID NO: 75 - DNA, 25 bp, Artificial
  primer sequence
atactcwaggcctayagcctgtggt

SEQ ID NO: 76 - DNA, 20 bp, Artificial
  primer sequence
ttgaggatgtagtgttttcc

SEQ ID NO: 77 - DNA, 27 bp, Artificial
  primer sequence
catttatagctgatcttctaacgcaag

SEQ ID NO: 78 - DNA, 27 bp, Artificial
  primer sequence
tgtcgcaggagtcttgacaggcctcag

SEQ ID NO: 79 - DNA, 25 bp, Artificial
  primer sequence
agcaggggccggctgaacctctggg

SEQ ID NO: 80 - DNA, 189426 bp, Artificial
  BAC clone containing Y-specific sequences (bovine)
ttatttttaaaaaccctattatacttttttctacattttttttgcctttcctgttcttcttttcccctgtggttaatgtttaatgcatataaatctttatctacgtcttttacttttgcgtatctattatttctttctcttctttatttcctttctgctcaacattttgttaattttttttttcattgctttattccccaattggcaccttgttccagttttgctttagttagttttcttctggtagatataattttggtttgctatgttagacagtttgatctattgtaactcattttacttggattctttagattttgcttatgggtgtatatgtgtatgtgtaaattccatcacactttttattgttgctataaactttggcctctatgttgggtttttacagttctgtggagttttccttttcttttcttttttctttttacatctgttattttttctctttataatttgaattaaaatttttttcaacgtattatatttttctacatttacccctttgtttgcctttcccactcttcttttccccttgcaattaacctttaatgtatataagccttctttgtctacctctattttattttgcatatctgttctctcttttctttctttgggtcctctctacatatttgtacttttgtttttattgctttatatcccacttagtacattatgttagttttcttttccagtttgtgctatagttagttttgtatttaactggtaaatataatttttgatttaccatctccactcaatctactgtagtttatttttgttggactcttttcactttgcttatgggtgtattggtatatatgtatattccattattttcattattatttgcctgatcttgtaactgccatttgtctggagttcatctttggactctcattttttgatatttcctaccatctcacttaatgcataacaaaccacttgtggaatcttcaatcataaccagagatcaagccctgagaattgaagtgagagcactgactccaagatcctagactacaagaaaactaaccttcgggaatatcaaatagtgagaactcacacaaaggaaacccttgaatacaatacctgggatcacccagccaccagcagcaccctatgctggacacctcacgcaaaaaacaagcaaaacaaaaataaaaacctaatcaacagcaagcaggattaccacatcattcagccttgcccatcagaggaaatacaaacaaacaaaaactcagcacgattctcattggaccaaacttaggagggcagaaaccaaaaggaagaaacwmcttywysckkgaagcctgagaaaacaagacctcaaacatagtaagttaaaaaaaaataatgaaaaggcagagaaatactacacaaatgaaagaacaagctagaaacacagaagtccaaataaatgaagaggaaataggcaaactacctgaaaaagaattcagaataatgatagtaaagatgatcaaaaaacttgaaaacaaaatggagaaaatctaagaatcaatgaagaaaatatagacgaattaaagaataaacatactgagacaaacaacacaattactgaaattaaaaatattctaaaaggaatcagtaacagaatatttgaagcagaagaccaaatctgtgagctggaagataaaatggtggaaataactgctgaagggcagaataaagtaaaaagaatagagaatagcctcagagacctcagggacaatatcaaacacactaacattcaaattataggggtcccagaataagaaaagaagaaatgatacgagaagttttttgaagagattatagttgaaaatttcccttagaaggaaaaggaaatagtcactcaagtccaagtgtaacaaagagcaccatacaggataaatccaaggagaaatatggcaagaaacatagaaagcaaactaacaaagactaaacagaaagaaatattattaaaagcagcaaagaagaagtaatgagtaacatacaaaggaaatcccatatgattaacagcttttctttcagcagaaattctgcaggcccgaggggaatggcagaatatatgtaaagttctgaaagggaaaaatctagcaccaggattacaatacccagctaggatctcattcaaaattgagaaatcaaaagctgtttagacaagcaaaagttaagacaatttagtaccaccaaaccagctatacaagaaatgttaaagggacttatattgtcaagaaatacaatagaataaagcaagtctacaaaatcaaccccaaacagttaagaaaatagctgttggaacatatataccaataattatgttaaatggattaaatgctctgaccaaaagacacagactggctgaatagatataaaaaccagacccatataaatattgtctacaagaaacccacttcagacatagaatgcaagtgagaggatggaaaaatatatttcatgaaaatgggaagaaaaagaaagctggagtagtagtccttgtatcagaaaaaatagacctgaaactaaagaagattataagagataaggaaggacactacataatgatcaaaggatcaatccaagaggaagacataacaattgtaaataactgtgcacccaacacaggagcaccttaatacataagacaaacagtaacagacacaaaaggagaaattgatactaacacaaacatagtgggagactttaacaccccactcatattgatggaaagatcatcaaaaccgaaaattaataaggaaacacatgttttaaatgatatatgagatgaaatgaatctcattgatatcttcaggggattccatcaaaatgcagaagaatgtaccttcttctcaagtgcacatggaacattctccaggatagaccacaccttggccacaaatcaaacctcagtaagtttaagaaaattgaaattatatcaagcatcttctcagatcacaatactatgaaactagatatgaattacaagaaaaacactgtaaggaacacgaacaaatggagattaaaccacacttttctaaataaccaataggttattgaagatatcaaaagcaaaatttttaaaaatccttgaaacaaatgacaatgaaaacctgacaattcaaaacctatagtatacatcaaaagcaattctaagagggaagtttatagcaatacaagcctacctcacaaaacaagaaaaagatcgaatagacaacctaacttgacacgtgaaacaactggaaaaaaaaacaaaaaataaaaaaggaaaaacaaaaattagtagaaggaaagaaatcataaagatttgagtagaattataagaaaatgaaataaaagagacaatagtaaagattaataaaactaaaagctggttctttgagatgataagcaaaattgacgacctttagctagagtgaacaagataaaaagagagaagaataaaaccaacaaaattagaaatgaaaagggagaggttacaactgacaatgcagaaatacaaaggattataagagactattatgaacaactctatgataacaaaatggacaacctgggagaaacagacagattgttagaaatgctcaatcttccaagactgaatgaggaagacagaaattaggcacaacccaattacaaggtctaaatcaaagctgtgataaaaaatctctcaaaagtctaggagcagaagacttcataggtgagttctatcaaacattagagatgacataatgcttatccttctgaaactctttcaagaaactgcagaggaaagaacactttgaaactaattccacaaggccaccatcaccctgatgccaaaaccaaagacaacaccaaaaaagaaaatgacaggccaatatcactgatgaacatagatgcaaaaaatcctcaacaaaattttagcaaacagaactcaacaatacatgaaaaagcacatacaccataatcatattgggtttatttgagggatgctagcattcttcaatatatgcaaattaatcaatatgacagaccatatttaccaaatgaaagataaaaatcatatgataatgatttttaatcattaaaaatcagaaatgcagaaaaaaaattagatttaaaaatgtaaaataattatattaaaaatgaagtttaaaaagaaatgaaaaatggaaaaaaaaaagttgaaatgactcaaatgtccatcaacaagtgactgaataaataaatcaatgcattgaaatattattcagccatcaaattaatggagttcttatacattctataacatgctatagttctgaaatgaytatgttgtttaaaagaagccagtaacaaaagcatgcatacttcattattccatgagtgcatatgtcccagattaggtaatcctctaaagacagaaaatagaggcatagataccagggcmcagatgaagtggaaaataggayatsyctgccaactggaccatgtttcccactggagtgatgaaaatattctagaatgaggcagtcattatggttatgcaacctatgaagaaattacaaatcaatgaattatacagttwaactgggaaattcatggtagatgaacaatatgtcaataaataaaaaaaattttttttgattactgaccttaacttctacgtccaggaatattatccttcaaaaataaagragragtagatattttcgtatgaacaaaaaattmssrsawwwakkttgagctgatgtctggtctgttggaaatgtaaaaaggcattatttactggataaaatgatattgaagagaaactatgagttaagagtgtcatgttgaagtttgctacaaaacaagtaaattctagaaagcaattatccttaaatgaataaataaattgaaaaaataaataaaggcaaattgaaatgaaggattattcttattattaattggtaaaagataaataatgggcttctccatctttacttttcactgatcatgaatcttcatagttaaagtgcatttcatatatacaagatatattttgactcagaagtaacgaatctgcctgcaatacaggagccatagcagacacgaattccatctttgtgccaggaagatcccctgcagaagagcatggcaacccactccagtattctcgcctggagaattcccaaggcaagaggagactggtgtgcttacagtccatggggtggcaaagagttggacacgactgagcaactaaacaacagacacagattcctgacagaaactctgaagatactagaaatacatgagaaagttctcagcctaaacaaagcacaaaaaccctagactgtagagctgaattataaaaaacctagacacaagagttgaattacaaaagattttaggcaataaaatgtctcccaacagtactgtaaacagtgtgttccacacaagggagaggagaaatttttaatactcattcaatagaacaacttttctagttacattcataaactaatgaagtaaaaatgtccagaaaataaacatagtataaaatctgttgatcatgttatatctaattaagactaaaattgtaagggaattttaaggaaaaaaaatgtgcaaaagatacagttaaagcatccacaatgaatgtaattttattttgttttttccatggtctcaaaataactgaaaaataagaatgtatttcattttaaaatatatttttcaaattcagcagtatattttattttcaaatataagcagattttgtacttttcaagctaaaaatgtttgtaccttgcagtgaatactttttgtttcactgattcagaggtaaataaaagcacgttatgtgtctttaccttgacaatttttgtggtatcactgtgtatttagaatcaatggtttaaacatggagtgtgtaatactacattccttcagggactgtaggcaatgtaggaagtggtcaaggccttccaaaccattaagtatgctctgtggaatggagtagtggtaaccatggcagcctgcgttggcaacaactgtttaacaaggtttgtcttagttaactccctcatagaagacaatgaatttttaaagataagtactaaatctgtggccatataaaatcatatatatatgattatacatgcatacaatatgtgtgtgcatatgtgtatgctgctaagttgcttcagttgtgtccgactctgtgcgatcccatagatggcagcccaccaggctccaccatccctgggattctccaggcaagatcactggagtgggttgccatttccttctccaatgcatgaaagtgaaaagtgaaagtgaagtcactcagttgtgtcggactcttagtgaccccatggactgcagccgtccaggcttctccatccatgggattttccaggcaagagtactggagtggagtaccattgctttctctggcatatgtgtgtatatatatatatgtatactaaaattttgttccttgtgaataataagtaaaacattaaaacctgacataaatgacaaaaactttttaaaacttaagccaacaaaatttgaatttgcaggagaaacatatttaattactgacattattttagaatactggagtatggagattgtaataaaaacagaaccagcttttacttgattttattcttgtaataaaactcctcgtgggcaatgcctcccagtgatgccagcactggatgttggccactccccctgacccacctgtggaaggccactgctccattcaaaattccttagctataaagggcctaatcagtattgcttttgtgacctacaaaaaccgcaattattttccttcctttctttcctcctacctctcccttttctttccttttctttcttattatacaattcataaagcttgcttcctgtttatagttattacaaaatactggcagtattccccatgttctacaatacattcttgagctcatcttacacctaacaccacctctgtgttgccccttctcctccccactatctgctggtaaccagttgatgttctttgtatctgttatattctctactttgtggtattttaagattctataagtgatatcataaagtatttgtctttcttattttacttgttgctgcaaaaggcattattccattctttttatggctgagtaacatttcataggtctttcttggtggctcagtggcaaagaatctgcctgtcaagaaggagatgtgagttaaaaacctaggtaaggtgtatcccttggaaaaggaaatggcaacccactagagtattcttgcctggaaaattccacaggcagaggagcctggagtgggtagagtccatagggtggccatgagtgggaaatgacttagtgactgaacaacaatgagtgtcccatagtacatacatactacacctttatacattcatccatcatctattggacatttagattgcttccatgtcttagcgaatggtatgcttcaaggaacattgggatacatgtctttttaattaatgttatgggtattcttttaatatatatatccaataatgaaactgttaggtcatgtgttccattcttagctttttgagaaatcttcatactaaaaaccatcattatttcattcctatggactatcttttagtaagcaagtttgagtttctgaggctgttaattgctgacatcacaccttgaggtgtaataaacactactatgaggtagatgccctaggggcaactactttgtatgccttttagcaaatggcactgccaccccacacatgacatggctccccagctaacttccctcctctgctttcacacacagaaacaccgtgtcctcttcctcttacatttcttcaaagaaatgtatcttttgggctcagtctgaacagagttctgtcccatactttctggttaactggaccttctagtcttgtctccaataccacaggacctatctccttcactcttcacacacttatcccttccctgcctctctgctccacaccatgtttcagagagttcatcagagctctcatcagagagctcatttcacatcaggttgattttatttaattttcattattaatatatgctgttggtagaagattcaatgtctgtagtgttggccatttctcagtggcgagacacccgaagaattctgagatctctagccatcttctcaagtcactagctgcccatatgtccagcttgaatggcagggctctccatgatctctggggcctcttccagtccgttctgattcagctcttcattcaccttggaacactggctacagagctttgaccctctaattttcttaagtttctctgtggtggaggctttctgaggagctttcttggtcagctttgcttgaatatgcttattgacccccatggccacctggagggtaaaaagagggagaacaaagaatggcattgttttgggaaaaagggcaatgatggtggtattgatggcttcaagagaagggatgtgggccaagaaggggcatgtgcaagccaaggacaagatagggtgtgtatgaggtggtgtcaatgggcaagtggagattgggcgggttcactcacctagacatttctttggacttctgttgataggaggtcttcatcttttcatcctggatataaggagttggctgttccataactgtgctagagccttttagcttccaagttaccttagtcattttgacgtctcccagtggtctgctccagggcccttgagcactcctatataggggagaccacacccttatgatcatgaggtcaggaagtcacttcctcagccagtggcggccctgggcaagtcttgacatcaaaaagcctcatcctgatacctctaagggaagattaggagcaactcagatgatttactgggagaaatgaagcatgtttaatgctcctagagagccctccaaactcacctgccatcttcatctctctaagacaacacagataggtcacatggtagagagtaagtgtcctccaaacctttccttacagccagttttcagcctttcattctgttctctctcatgtttaactgttatccagattttattatcttgcctaaaatccttccatggtaattaagcagtggaaatgagaatcatttcacttttctcctacaagtggtcttaaagcatcttgaacaccattaaattctgaaccacctaccagaaaccttttccatcccacccccttttctaagtgttcgcatacctttcctcgattttctagtttccagtctgaaacccctgtcttcggtctgtgagactgcactgactacacttgctcttgttccaattggagctgtcttgtttcaaatttaggactcatgtgccggagtccaactctagcaaccacagattcaacttgaaaaggtgaacagtgttggccaatgagacagcctctcagttttctatggactgcctgtttatttcaagtttaagattatcttttacacttttacaaaaacattgggccagaagtttgacattttcagttccccatctcccagatatattatctccataaatcattgtcgctcttcaaacagagttcctgcttcagtgattctctcagaatcagccctcctctaatgtgtcctatagttaacttatgattacattgtaactcatgctacattcctcagtttactacttatcttcctaaatcctgtctgcccttgatatctcgagctcactatctcttgaaaaagacttctagctatagtgtctaaaaatccctaacttctataaactatagtaaaatatgctaactttacaacattccttaaatctttaacttctaactattttaattatttctgagccctaaattcagtaaattcctttgccataaacattttcctcacaaatagacttcagatatcaatccctcccatggcctcaagctacggcctatgtgctcatcctggaacactcttttgtaaaagtccttgaacaaatgtcaatgattaactttatgaattattttctgagcacagctgcagaaggctttgtgcgttctcatgctcctctcgagaacaataagcaccttaatattccttttcagtcaactcagccgacgagaggaaaagacaagtcagaattacaaggcctaactccttcatcccaggatccatgcctgcagaatgaggagaggggtctgagggccgtgcctccattttgttagtaatgcctaaggcagctcctgacactcatggtcacctcactcggtataatacccaggcttcggtttcaacaggttggtggggatgaggcagagaaatgcatgcttcaggttgcactgaatgatgccaagaaggaaatgaccacctgtgactgatcagtattcccaggaagaggtagcacttgcccagtggtatgtgagcctcatgtgcaaaggtgttctcaacatgtgccctcacaaatctcttttcttatcctgtgttcctgccagcacctcctttggaatttacctcacagtcgagaccatggtggaattcattttcatgaatagccaccacaagcttccagcttcttggaggaaacactgagtaatcatgtcctgcccatctagttcccaagatctctcattgaggttgttggcactggaggatacgggaattactggggtccagccccggttggatccacgtattccttgggaggatggcgttggcaaaatgatagataaaaggagaaaaagacagaggcttgaactaactggtttacgcagaaagccaataaaacctgtgacatcaggtttgcactgaccacgcaggccacaggtgccctctcaaatcgtagaaggtgcccactttaggcaccttctcgagtgggtcttagaagcctgggcaaataagtggtctcagaggtcccccacactccaaattagtcwtcctgaaggaagaacagagaagaaaaggagagaaaaggaaacaagaaaraacgacacagcgagacctagcttgatgagcatggcctgcaactttattttccaaagtagcttttataccttaagttgtgcatagaggataatagggggtgtagagtcatgcaaggtcggcagtccttgactcttatcgaagccaggctttctttctgcaaacttatcacdtgcaaaggctttaggtgatttacatcatcttctggccaggaggcctgttaacattttatgaccctttcttctgaataatggttagtcaatcagaaaacttattttctctaaaggtgattattctaaagtcaggcgccaccctctgaaagcattagataaagttgcattcctatagggcaaaagtgtggtgggttataacaagaaaagaattaactcaagggtccaaggttacaaacattaaagctactacttacatttctatataccaactatcttaatcaatacacacccagggacacagtagttaagggatatggaaacttggcagcacgcattagctcaacaaagagatcctctactagttctattctaacaattttaactctctgagaagctctgcattgttagaatatcttaagcttcccgtgcctcttgtggttgggaggctgtgaacaatcacatgcatacctgcaggagtccaaacaaacctgtcaggcaagcttgaaagtcatcagaggggtttgaattgaaacactcctattatgcccaggagacttattaactagagccctaagttgattttcttcagagaaaggtggtcggggatagccccccattaatgtcagaagagttggtgaaagtcgtgaaatagtaaaacagacagattttggttttggggtagatggttgggcatatccagggggcctctagagttctgattcacctttgcatgtcagatcctctctgcwtgacctttgtcatgggtgggaactcctgtgctggcttccagcagggaatgagttgtgtgcttcactgcagtccttgtattcctaattgtccagcagggtcagccagcctgagatgtgcccctgggggacactgttcattggcttagtgggacttcctgtgatttctgtgaagtgctcacttcgtggtgtccactggttgcttattcacccaggacacatcacactgctcaaaattttaagaggtagaacagcacaccataaacaaaccgaaaagatgactcaatatttttataacaaagaacctaaaacctgatagaaaaacgtgaaaagacattggccaacagtttctaagaaaactgtaacactgacctttaactttattaagagatgtttaaaagacatgagaggcaattggcaagaaaggtatggaactagtcgctaaccacattaaaaagatatgtaatctcactaaggtaagaaatatacttggatgcaagctcaaaaatgacacaatgatctctgttcatttccaaggcaaagcattcaatatcacagttttctaagtctatgccatgaccagtaatgctgaagaaactgacaatgaatggtactatgaagacctataagaccatttagaactaacacccaaaaaaagatattcgtttcattataggggattggaatgcaaaagcaagaagtcaagaaatacctggagtaagaggcaaatttgaccttggagtacagaatgaagcagggcaaaggctagatagcatattgaaaagtggaaacaatttcttttgtgggagctctcaatgaccttttatatattccatggacacagagtctgcctagctgattatgtggatttaatctgcaacttttatgctggtaggaaggtttgcattttcttttttagccacactgaccacagtgggtttccattgtggttttatttccatctccccatatgattcattcactggggtttgctcctgaggctgccctgcaggaattgggtttgcccctgggacatgtggagagaataacctttgccctgcttcattctgtactcccaaatgctgctgctgcttgggtcacaggcattttggcagcaccaggtactcaggacggtaggcactgtagagagggcatcagagggcatcagagggcagacggactgaaaacacaatcacaggaaactagccagtctgatcagagaaggcaatggcaccccactccagtactctcacctgcagaatcccattgagggaggagccaggtgggctgcagtccatggggtcgagaaaatgagacatgaccaagcgacttcattttcacttttcactttcaagcattggagaagaaagtggcaacccactccagtgttcttgcctggagaatcccagggacgggggagcctggtgggctgccgtctatggggtcgcacagagtcggacatgactgaagtgacttagcagcagcagcagcagccggtctgaaaacatggaccacagccttgtctaactcaatgaaactaagtcatgccttgtggagccacccaagatggagaggtcatggtggagaggtctgacagaatgtggtccactgcagaagggaatagcaaacaacttcaatgttcttgccttgagctctgttcgtttccaaggcaagccattcaatatcacggtaatccaagtctatgccccaaccagtaacactgaagaagctgaagttgaacagttctatgaagacctacaagactgtctagaactaacacccaaaaaagaaattcctttcattataggggactagaatgcaaaagtagaaagtcaaggaacacctggaataacaggaaaatttggccttggagtacagaatgaagcagggagaaagctaatagacttctgtcaagagaacacactaatcatagcaaacacccccttccaacaacacaagagagactctacacatggacatcaccagtggtcaacaccacaatcagattggagaagttctatacagtcagccaaaacaaacaagactgggagctgactgttgcttagatcatgaactccttattgccaaattcatactgaaattgaagaaagtggaaaaaccactagactattcacgtatgacctaaatcaaatcccttaggactatacagtggaaatgaaaaaaggatttaagaaactagatctgatagacagagtgccggatgaactatggatggaggtttgtgacattgtacaggagaaaggaatcaagaccatcccaaagaaaaaaatgcaaaaaaagcaaaatgtctgtctgaggaggccttacaaatacctgtgaaaagaagggaagtgaaaagcaaaaagaaaaggaaatatacatccatttgagcgcagagttccaaagaatagcaaggagagataagaaagcctttctcagtgatcaatacaaagaaatagaggaaaacaatagaatgggaaagattggggatctcaccaagaaaattagagatacaaagagaacatttcatgcaaagatggtctcaataaaggacagaaattatatggacctaacagaagcagaagatattaagaagaggaggcaagaatacacataattgtgcaaaaaagatattcacaacccagataatcacaatggtgtgatcactcacctagagccagatatccttgaatgtgaagtcaagtggcccttaggaagcatcactatgaacaaagctagtggaggttatgggatttcagttgagctatttcaaatcctcaaagatgatgctgtgaaaatgatgcactcatatacgccaggtgcactcaacacgccaggaaatttggaaaactcagcagtggccacaggacttgaaaaggtcagttttcattccaacaccaaagaaaggcaatgccaaagaatgcccagactagtgcacaattgcactcatctcacatgctagtaaagtaatgttcaaaattctccaagccaggcttcagcaatatgtgaactgtgaacttccagatgttgaagctggttttagaaaaggcagaggaaccagagatcaaattgtcaacatccactggatcatggaaaaagcaagagagttccagaaaaacatctatttctgctttattgactatgcaaaagactttgactatggatcacaataaactgtggacaattctgaaagagatgggaataccagaccacctgacctgtgtcttgagaaacctgtatgcaggtcaggaagcaacagttagaactggacatggaacaagagactggttccaaataggaaaaggagtatgtcaaggctgtatattgtcaccctgcttatttaacttctatgaagagtacatcatgagaaatgctgggctggaagaagcacaagctggaatcaagattgcaggaaaaatatcaataatctcaaatatgtcgatgacaccacccttatggcagaaagcgaagaagaaaagagcctcttgatgaaagtgaaagaggacagtggaatatttggtttaaagctcaacattgagaaaactatgatcatggcatccggtcccatcactttcatggtaaatagatggggaaacagtggaaacagtggccaactttattttggggagctccaaaatcactgcagatggtgactgaagccatgaagttaaaagacgcttactccttggaaggaaagttatggccaacttggacagcatattaaaaagcagagacattactttttcaacaaaggtccatctagtcaaagttttggtttttccagtagtcatgtatggatgtgagagttggactctaaagaaagctgagtgccaaagaattcatgcttttgaactgcagtgttggataagattcttgagagtccctgggctgcaaggagatccaacaagtccatcctaaagcagatcagtccaaggtgttcattggaaggactgatgttaaagctcaaactcctcacatgaagagctgactcattggaaaagaccctgatgctgggaaaaattggaggcaggagtagaagtggacgacagaggatgaaatgtgtggatagcatcaccgactcaatggacatgggtttgggtagactccagcagttggtgatggacagggaggcctggcatgctgtaattcatggggtcacaaagagtcagactcaactgtgcaactgaactgaacaggctagtctgtcatttgactgagccaatggaataggccatgaccacacaggctgtcggccatttgattgaaagcactgtttaggccaggcccaaaaaggctagtcttcctttccattgacaccactgattaggccaggcccacacaggctagtatgacttttgtttgagaccatggattagactagtaccaagacgctagactgtcatttggctgagaccatggattaagccagggtcacactgtctagtctgccatttgattgagaacatggattaggctactaccaacaggcttgtcttcatttgactgagaccatggatttggccaggaccacccaggcaagtctgcctttcgactgacaccactaaatagtccaggcccacccaggcagactgacttttgattgagactacagattaggccagtaccaaaaggctagtttgtcattcgactgagaccatggattaggccaggcccacccaggctagtctgcctttaaattgagaccatgaataaggccaggaccactgaggctaggcttccatttgttttagggcactgcttaggccaggcccacccaggatagtcggcctatcaattgacaccactgcttagggaaggccaacccaggctagtcagccttttgattgagaccacggattatgccagtaccaacaggatagtctctcatttgactgagcccacggattaggccaggaccacccaggctagtctgttatttgattgagagcactgcttagcccaggatcacctgggctagtctgcatttagatagaaaccagggattaggtcaggccttcactggcgagtcagcatattgactgacaccactgattaggccaggcccactgaggctagtctgtcatttgactgacatgaaggattagccaggccaatccagtctagtctgccatttgattgacaccatggattaggccaggacctcccaggctagtttcccatttgattgagagcattgcttagtccaggcccacctaggctagtctgtattttgatcaaaaccatggattaggtcaggcccacacaggcgagtctaacatttgattgagaccacagattaggccagtaccaacaggctagtctgtcatttgactgtgacaacggattaggccaagcccacccaggcgaacctatcatttgattgagcccacggattaggccattcacacccaggatagtctgccatttgattgcaaccagtgcttaggccaggcccacccatgctaatctgcccatgaattgagaccacagattagtctacccattctagtctagtctgccatttgattgagagcactgcttaggccaggtccaccaagctagtctgcattttgatcaaaaccatggaataggtcaggcccacacaggagagtctgcattttgactgacaccgctgattaggccaggcccacccaggctagtctgcctttgaaatgagaccacggattaggccagggaaacctaagctactcttgtatttgtttgagagcagtgcttaggcaaggcccctccaggctagactgcatttcgcatgacccaactgatatggccatgcccacctgggcaagtcagagatttgattgagtgcattgcttaggccacagccaacaagctagtctgcgttttgatagaaacctggattcggccagtgacaacaggctagtctgtcatttgacgagaccaatgaataggacaggtgcacccaggctagtctgatatttgattgagagcacagcttaggccaagaccacccaggttagtctgcatttcagatgacaccactgactatgccaagtccacccaggcaagtctgccttttgattgacaccactgattaggccttgcccacccaggctagtgtgcaatttatttgagtgcactgattaggaaggcctacccaggctagtcagccttttggttgacaccacttatcaggccaggtccatccaggttactttgcctttgaattgagaccatggatgaggccagtatcaacaggcaagtctgttatttgagcaagaacatggattaggcgaggctcacacacactagtctgccatttgagtgatccatggattagccatgcccacctgggctagtcggccatttgattgagagcactgattaggctagggagacccaatctaggctgcattttgattgacatcactgattaggccaggcccacccaggctagtctgccttttgattgagagtgctgctaagaccggtcccacccaggctagtctgacttttgattgagaccatggattaggtcagtattaacaggctagtctgtcatttgactgagcccacagataaggccaggaccacccaggctagtttgcgttttaattgacaccactgattaggccaggcccacccaggctagtctgcctttgaattgagaccacggattaggccagtactaacaggggagtctgtcatttgagtgagaatatggattaggccatgctcacccaggtagtctgccatttgattgagagcactgcttaggccaggccctcccagattagtctgccttttgattgagattacagaggccagtaccaacaggctagtctgccttttgattgaaaccactgattagtccaggaccacccattctattctttcttttgattgacactgctggttaggtgaggccaacccaggctagtctgccatttgattgacaccacatatgaagccaggcccacccagggaagtctgccttttgactgagaccatggattaggccagtaccaacaggtaggtctgtcatttgactgaacacagattagaacagatctcccaggctagtctgccctttgatcagcaccacggattatgccaggctcactcagatgactctgcattttaacagacaccactgatcaggccaaacccacccaggctagtctgtcatttgagaccacggataaggccagtaacacccaggctattctgcctttcacttgacaccactgattacgccagggccacccaggttagtctgcctttcgactgagacatggattagataggcccacccagtctagtctgcttttcagttgacaccactgattatgccatgcccacctaggctttctgcaattcaattgagtgcacttcttaggccaggcccacccaggcttttctgtgatttgactgagccatgaattaggccattcccacccaggctagtctataatttcattgggtgcactgcttaggccaggcccgcccaggtgggtctgcattatactttagtctgttaggccagtaccaacaggctagtctgccctgtgattgaaaccatgtattaggccacggccccaccctccaagttaggctgtgaattgattaagagcactgcttaagcaatgtccacccaggctggtctgccatttgactgagccaccaattaggccaggcccacccaggcaagtctgccttttgaatgagaccacagatcaggccagtatcaacaggctagtctgtcatttgactgagcctatggattaggccaggcccagccaggctagtctgccatttgactgagccatggattacaccaggctcactcagatgactctgcattttgactgatagcactgatcaggccaggcccacccaggatattctggccacttcagttagagcactgcttatgccagggccacccagcctattcaagcttttgattgagaccacaaaatatgccagtaccaataggctggtctgtcatttgattttgaccatgatttagaacaggtccacccaggctagtctgccctttgattggcccatatattatgccaggctactcaagcaattctgccttttgacagacaccactgatcaggccaggcccactgggctagtctgctatttgactgagaccacggaatagggttggaccacacaggctactctgcctttcgatttacaccactgattaggccaggcccacccaggctagtatgccttttgtttgagactatggattaggccagtaccaacaggctagcctgtcatttgagtgaccatggaataggccaggaacacccaggatagtctgcctactgactgggagggctgcttacgcaaggcccacccaggctagtctgcctttcgatgaaaccattgattaggctaggtccacccatgctagtctgccatttgattgaaaccacatatgaggccagagccacctaggaaagactgccttttgattgagaccatggattaggccagtatcaacaggctagtctataatttgactgagcccactgattaggccaggccacccaggatagtctgccatctgactaaccacggattaatcagactcattcaggcaactcagcatttttaatgataccactgatcagacctggtccacccaggctattctgccattttatttagagcactgcttatgccaggcccacccagactagtttgcctttcaaatgagtgtactgcctaggccaggcccacccaggctagtctgccttatgatttagactgattaggccagtaccaacaggcagtctgccctctgattgaaaccatgaattaagtcatgcccacccaggttagtctgcaaattaattgagtgcactgcttaggcaatgtccatccaggctaatctgccatttgactgagccaccaattaggccaggcccacataggcaaatctgcctttttaatgagaccacagattagtccagtaccaactggctggtctgacatttgactgagagcaggattagaacaggcccacccagccagtctgctctttgaccagcacccagattatgtcaggctcactcaggcatctctgcgttttgacagacaccactgaacaggccagacccacccaggctagtttaccatttgattgagaccatggattaggccagtaccacccagatactctgcctttagattgagaccacagattaggaaaggcccacccaactagtcttctttttgattgacggcattgattatgccatgcccacccaagctattctgagtttccactgagtgtactgcctaggccaggcccacccaggctagtctgccttatgatttagactgattaggccagtaccaacaggctagtctgccctccaattgaaacgatgaattaggccatgcccacccaggttgttctgtgaatagattgagtgcacaatttaggcaatgtccacccaggctagtctcccatatgactaagccaccaatcaggccaggaaacccagtcaagtctgcctttttaatgagaccacagattagtccaggcccacccaggctattctgccttttgattgagaccacagaatagatcagtaccaaccagctggtctgtcatttgattgagaccacaatttagaacatgtctacccaggctagtctgccctttgattgggcccatggattatgccaggctactcaagtgattctgcattttgacagacaccactaatcaggccaggcccaccaggctagtctgctgtttgattgagaccatggattaggccagtaccacctaggcaactctgtatttcaattgacaccactgattaggccaggcccacccaggttattctgccttgatttggaccacgttttaggccagtaccaacagtacggtctgtctttttattgagatcacagaatagaacaggcccacccaggctagtctgccctttgatcggcaccacagattatgccaggcccactcctgtgactctgcattttgacagacaccaccaatcagaccaaactcacccaagctagtctgccatttgactgaggccacagatttggccagaaccacccaggctactctgtctttctattgataccacataataggccaggcccacccaggctagtctgctgttcaattgacaccactgattaggccagaaccaaccagctagtcttcctttgatcgacaccactgattaatccaggcccacccatgctgttctgcgttttgactgagaccatggattaagccagtaccaacaggctggtctgtcatttgattgagaccataggttagaacaggtccaccaaggctagtctgtcctttgattggccccatggattatgccaggctactcaggcaatgctgcattttgacagacaccactgatcaggccaggcccaccaggctagtctgtcatttgattgagaccacagattaggccagtaccacataggctactctgcatttcaattggcaccactgattaggccaggcccaccaggctagtctgccatttgattgagactatggattaggccagtaccacctaggctactctgcatttcaattggcaccactgattaggccaggtccacccagtctagactgtcatttgactgagactgattatgccagcaccaacaggctagtctgctattgccttagaccacggattcggccatgaccaacaggctagcctgtcagttgactgacaacagaataggccaggaccacccagggtagtctgcctttcgattgagatcatggattaggatagaccccccaccccgcaccaggctattctgcatatcgattgagtacactgcttaggccagacctacccaggatcttctgtgatttgactgagccacagattaggccattcccacccaggctggtttacgatttgattgggtggactgcttaggccaggcccacccaagctagtctgccttatgatttagactgattaggccagtaccaacaggctagcctgccctccaattgaaactatgaattaggccatgcccacccaggttggtctgcgaatagattgagtacactgcataagcaatatccacccatgtcctaccatttgactgagaccgtggattatgccagtatcaacaggctaatctgtcatttcactgagcacacagattaggccaggtccagacaggctagtctgccatttgactgagccacagactatgccaggctcactcaggcaactctgcattttgactgacaacactgatcaggccaggcccacctagaatattctgccacttcatttagagcactgcttatgccagggacaccttgctagtctgcctttcctttgatatcactgcttataccaagcccacccaggtattctgccttttgattcggaccatagaataggccagtccaacaggctggaatgaagaggaactaaaaagcctcttgattaaagtgaaaaaggaggagagtgaaaaagttggcttaaagctcaatattcagaaaactaagatcatagaatctgatcccatcacttcatggcaaatagatggggaaacagtggaaacagtgtcagactttatttttctgggctccaaaaccactgcagatggtgactgcagccatgaaattaaaagacacttactccttggaaggaaagttatgaccaacctagacagcatattcaaaagcagagacattactttgccaacaaaggtccatctagtcaaggctatggtttttccagtagtcatgtatggatgtgagagttgaactgtgaagaaagctgagcaccgaagaattgatgcttttgaactgtggtgttggagaagactcttgagagtcctttggactgcaaggagatccaaccagtccattctaaagtagatcagtcgtcggtgttctttggaaggaatcatgctaaagctgaaactccagtactttggccacctcttgagaagagttgactcattgcaaaagagtctgatgctgggagggactgggggcaagaggagaaggggatgaacagaggatgagatggctggatggcatcaccgactcgatggatgtgagtttaagtgaactctgggagttggtgatggacagggaggmctgatgtgctgcaattcatggggtcgcagagtcagacacgactgatcaactgaactgaactgaactgaacagactggtctgttatttgattgagaccacgatttagaacatgtccactcaggctagtctgcctttgatcagccccacagattatgccaagctactcaagcaattctacccatcaggccaggcccaccagtctagtctgccatttgattgacaccatggtttaggccagtaccacctaagctactctgcatttcaattgacaccactgatatgctaggcccacccaggctattctgccttttgattgagaacatggattagtccagtacattgattagaacaggcccaatgagcctagtctgccctttgatcagcaccatgtattatgccaggatcactcaggtgactctgcattgacagacaccgctcaataggccaggcccaccaggctagacaatgatttgattgaaataatgtattaggccagtaccacccaggctagtctgccttttgattgacactgattaggccagtaccaacaggctagtccactatttgactgagaccatggaataggtcaggaccacacaggctactctgcctttccattgacaccactgattaggccaggcccatcaaggctaatctgttgttcgattgacaccactgatgaggccaggaccacataggcgagtctgcctttgattgacaccatgtattaatccaggcccacccaggctattctgccttttgattgagatcatgtattaggctagtaccaagaggctggtgtgtaatttaattgagacaacagattagaacaggtccaccaaggtgagtctgccctttgatcggcccacggattatgcacaggcttctcaggtgattctgcattttgacagacacttctgatcaggcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnattatgccaagcttctcaggcgattctacattttgataaacaccactgatctggccaggcccacaaggctagtctaccatttgattgagaacacagattatgccagtaccacctaggctactctgcatttcaattgacaccaatgattaggccaggcccagcccggctagtctgccttttgattgagactgattatgccagtaccaacaggctagtctgctattggactgagaccacggaataggccaggaccacacaaagtactctgcctttcaattaacatcactgattaggccaggcccacccaggattgtctgtcttttgattgagacacgaattaggccagtaccaacaggctatattgtcatttgactgaccaaggaataggtcatgcccacccaggctggtctgcaatttgactgagcaccaattaggccatgcctatccaggctggtttgctatttgattgagagaactgcttaggccagcctggtcaaggctagtttgcatttcaattgacactactgattaggccaggcccacccaaggaagtccaccttttgtatgagaccatggattaggccagtatcaacaggctagtctatcatttgactgagcccacagatgaggccagaggcactcaggatagtctaccatttgactgagtcacagattatgccaggctcactcaggtgacaaggcattttgactgacaccattgatcaggccaggtccacccaggttattcagccatttgatttagagcactacttataacagacccacccaggctagtttgcctttcagttgagagaactgcttatgctaggcccacccaggctagtttacccctcaactgacactgctgataaggccaggaccacacaggctagtctgtctttcaattgacaccactgattaatccaggcccacctaggctattctcccttttcattgagaccatggctggtctgcaatttgagcaccaattaggccatgcctatccaggctagtttgctatttgattgagagaactgcttaggccagcctggtcaaggctagtttgcatttcaattgacactactgattaggccaggcccacccaaggaagtccaccttttgtatgagaccatggattaggccagtatctacaggctggtgtgtcatttgactgagaccacggattagaacagacccacctaggctagtcttcactttgatcgacaccgcggattacgccaggctcactcaggagactgcattttcacagataccaatgatcaggccagacccacccagggtagtctgccttttgattgagaccacagattaggccagtatgatccagactactctgcctttcaattgttaccacgattaggcctggcccatccagcctagtctgcctttwaatgagaccacagattaggccagtaccaacaggctgcactgtcatttgtctgaccagggaataggccagggaatagtcaattggctagtctgccaattgactaggaggactccttcggctaggcccacccacgttagtctgtcatttgattgataccacatatgaggccaggcccacccagggaagtctgccttttgattgagaccatgtattaggccaataccaacaggctggtctgtcatttgactgagaacatggattagaacaggcccacccaggctagtctaccttacgattgaggccacggattaggataggcccacccaggcaagtctgccttttgagtaagaccatggattaggccagtatcaacaggctagtctgtcatttgacagagactatggattaggccaggcccagccaggctagtctgccatttgactgagccacggattatgccatgctcattcaggcgactctgcattttgactgacaccactgatctggccaggcccacccaggatattctgcctcttcattttgagcactgctcatgccaagcccacccaggctattctgccttttgattgacaccacagaataggccagtaccaacaggctagactgtcatttgattgagaccatgaattagaacaggtccacccaagctagtctgccctttgatcggcaccacagattatgctactcagacaactcagcagttcaacgacaacactgatcatgccaggcctaccaggctagtctgccatttgattcagaccatgaattaggtcagaccacctaggctactctgcatttcaattgacaccactgcttaggccattcccacccaggcaagtctgccatttgactgagagcactgcttagcccagcctgcacaggctagttcatattttcgattgacaccactgatttggccagccccacgcaggctagcctgccttttgactgagactgactaggctagtaccaaaaggtacttctatttctacttctacttctagtcttctatttgactaagaccacggaagaggccaggaccacacaggctattctgcctttcagttgacaccactgattaggccaggcccacctaggctactgtgccttttgatcgagaccacaaattggccagtaccaacaggctagcctgtgatttgactgaacatggaataggtcaggacaacccagcccacccaggcaagactcccttttgattgagaccacagattaggccagtatcaacaggctagtctgtcatttgactaagcccgtggattgggccaggcccacccaggctagtctgccatttgactgagccacagattatgctgggctcactcaggtgactctgcattttgactgataccactgatcaggccaggcctactcaggctagtctgcttttgattgagaccacggattagaccagtaccaacaggctggtctgtcagttgactgagaccatggattagaataggctagtaggccctttgatcagcacctcagattatgccaggctcactcaggtgaatctgcatttcaacacacaccactgatcaggccaaacccacccaggctagtctgccatttgattgagaccacagattaggacagtactatccaggctactcagcctttcgattgacaccactgattaatccaggcccacccaggttattttgccttttgattgataccatgaattaagccagtaccaacaggctggtctgtcattttattgagaccctggattagaacaggtagacctaggctagtctgcctttcgattgagaacacggattaggctaggcctacccaggttagtctgccattgtttgacaccactcattagaccaggcccaaccaggctagtctgcctttcaattgaaaccacatattatgctaggcctacccaagctagtctggcttttcatttacaccactgattacgccatgtcagcccatgctagtctgcaatttgattcagtacactacttatgccaggcgtacccaggctagtttgtcatttgactgagccatggatttggccattcccccctggcaactctcccatttgattgagaactctgcttaagccaagcccaccccaggctagtctgccttttgattcacaccactgattaggccaggtccacccatgtttttctgccttttgattgagacctcaaattagaccagtaccaacaggctagcctgtcatttgactgagaccacggattaggccaggcccacccatgctattctgcactttgataggcaccatgggttaggccaggcacactcaagcatctctgcattttgatctacaccactgattaggccaggacccccaggccaatttgcctttcaagtgagaccacagggaggccagtaccacccagtctagatagcctttggtggattccatggattttggcaggccccccccaccgccccccaggcaagtctgccatttgattgagagcactgcttacaccaggcccacccagactagtatgccttttggttgacagtactgattagttcaggcccactcaggctagtctgccttttattcagaccttggattaggctagtatcaacagcctagtctgtcatgtgactgatcccacagattaggccaggaccacccaggttagactgtcattccaatgagccattgattaggccatgcccacccaggctagtctgacatttgactgagagctctccttagcccaggtcctcccagggtagtctgcattttgatagacaccactgattaggccaggccaaccaagtctagtctgcctttctattgacaccactgattacgctaggtccacccaggctattctgccctttcactgggcccaaggattatgccaggctcactcaggcgactctgcattttgaaaagcaccactgaccaggtcagcaccaccaaggctattttgccattggattgagaccacggcttaggcaagtcgacccaggctactctacctttcaaatgacaccactgattaggccaggcccacacacacaggctagtgtctttcgattaacactactgcttaggccaggcccacccggctactctgccattggattgagaccacgtattaggtgcataccaacaggctagcctgtaatttgactgagaccatgtattaagccaggaacacacaggctactgtctttctattgacgccactgattacgctaggctcacccaggctttctacctttcgattgggaccatggattaggctttgctcatgcaggctagtctgccttctgattgacaccactgattaggccatgcctactcaggttaggttgcgaattgtttgagtatactgcttaggccatgcccacccaactagtctgccatttgattgtgagcactgctttggccaggcccacctaggctagattgcatttcaattgacaccactgattaggccaggacaacccagccatgtctgccttttgattgaaaccacgaattagggcagtatccacaggctagtctgtcatttgccccacggattaggccaggcccacccagggtagtttgtatttcagttgacaccactgattaggccaggctcacccaggtaagtctgcttttcgattgaaaccatggattaggctaggcccacccaggctagtctgtcatttgattgagagcactggttaggccaggaccacacaggctactctgcctttctgttgacagcactgataaggctaggcccacccaagcttgtctgatttacgtttgagaccatggattaggctaggtgcacccaggctagtctgccctttagttggcaccacggactatgccagattcattcaggtgactctgaattttgacagacaccactgatcagtccaggcccaccccagctagtctaccatttattgagaccatggttaggctagtactacccaggcttttctgccttttgattacaccagtaattaggccaggcacacacaggctagtctgcctttcaattgacacaactgcttaggccaggcccaaccagactagtctgccttttgattgacaccgattaagccagtacctacaggctagtctgccatttgcctgagaccatggaataggccaggaccacacaggctactctgactttcaactgacactactgattaggccaggccctcccacatagtctgcctttcaattaacagtgctgattaagccaggtccaccctggctagtctgctttttgagtgagacccaggattaggctgggcccacccacgccagtctgccctccaattgacaccactgattaggccatgcccaccgaggctagtctgccatttaactgagccacattttatgccatgcccacccaggctagtttgccatttgatcgagagcaccgcttaggccaggcccaccctggctactttgcatttcagttgacaccactgattaggcaaggcccactcagtcaagtctgcattttgattgagaccacagattgggccagtatcaacaggctagtctgtcatttaaactaatcccaaggattacgccaggcccacataggatagtctgaaatttgactgagccacggattatgccaggctcactcaggtgactctgcattttgatgacactactgatcaggcaaggcccacccaggctagtctgccatttgatttagaccacagattaggccagtaccaccctggctactctgccttcaattgacaccactgattaggacaggaccacccaggctagtcttccttttgattgaaactgattaggccagtagtaacaggctagtttgccatttgactgagaccatggaataggccaggaccacacaggctactctgcctttcggttgacaccactgattagaccaggcccacccacgctagtctgcctttcaattgacagcactgattaggctaggtccaccccagctactctggcttttgattgagaccatggattaggccagtacgaacaggctagtctgtcatttgatggagactgtggattaggtcaggacacacaggctactctgactttatattgacaccactgattaggctaggcccacacaggctagtctgccatttgactgakgtcacggattatgccaggctcactcaggtgactctgcattttgactgacaacacttcaggccaggccaacccaggctactctgcctttcgattgagaccatggatgaggccagtmccaacaggctagtctgtcatttgaatgagaccacagcttaggccaggaccacacaggctactttccctttcatttgacaacactgatgatgctaggcccacccaggcttgtctgcctttcgattgacaccacagattcagcaaggcccacccaggtagtctgcttttgatcagcaccacagattatgccaggctcactgaggcgacgctgcattttgacagacactactgatcaagtcaggcccaccgaggcaagtctgccatttgactgacacgacagattaggccagtaccacccaggctactctgatattcagttgacaccactgcttaggtcaggcccaccctggctagtctgccttctgattgagaccacggattagtccagtatggctcttgatgaaaatgaaagaggagagtggaaaagttggcttaaggctcaacattcacaaaacaaagatcacggcatctggttccatcacttcacggcaaatagatgaggaaacagtttcagactttatttttggggctccaaaatcactgcagatggtgactgcagccatgaaattaaaagacacttactccttggaaggaaagttatgaccaacctagacagcatattcacaagcagagacattactttgccaacaaagttccatctagtcaaggctatggtttttccagtagtcatgtatagatgtgagagttgaactgtgaagaaagctgagtgccaaagaattgatgcttttggactgtggtgttgaagaaaacttttgagagtcccttggactgcaaggagatccaaccagtccattctaaagatcagtcctgggtgttctttggaaggaatgatgctaaagttgaaactccagtactttggccacctcattcgaagagttgactcattggaaaagactctgatgctgggagggattgggggcaggaagaaaaggtgacaacagaggatgaggtggctggatggcatcaccgattctacagatgtgagtttgagtgaactccaggagttggtaaaggacaggaggcctggaaggttctgtgattcatggggtcccaaagagtcagacaggactcagggactgaactgaacaggctagactgtcacttaaatgacactgcatattaggctagcaatggcaccccactccaatactcctgcctggaaaatcccatggacagaggagcctggtaggctgcagtccatggggtcgctcagagtcccacaggactgagcgacttcactttcacttttcactttcatgcattggagaatgaaatggcagcccactccagtgttcttgcctggagaatcctggggacggcggagcctggagggctgctgtctatgcacagagtcggacaggactgaagggacttagcggcagcggcggctgccctccgattgacaccactgaaggggtcatgccgaatcaggttagtcgcgaattgattgactacactgcttatgccatacccacccgcgcaggaatggggggcggggggtggggagggctcactgcgcgtgctaccagggaagggctggccccgcacgagcgcaggaacgtggcgggggagggaacggggggtggggggtggcggctagtgcccctccacgcgcagcaagtggggttttgcaccacccatgccaggaggcctgggcgtacttaagaagtggggtctcacgtcgcgcaacgcacgcacagtgagaaggctgataccgtggcccagcaagagggcctcgtgtcgcctgtggcgtggggccccgtgcgcgcaagaaaggagagctcgcgccctgcagcgtgggcagaaaaggggggcctcgggccgcgcgtgccggaggagggctggcctgcatgcgcagtaagagggctctcgcgccccgccgcgctggcaggaacgggggtgcttgcgctggcaggaacgtgggtgcttgcgctgcctgggcaggaagggggggtttcgcgccgcgcgtgccgaggagggcgggggactgacacccggctcgcgcaggaaccggggtctcgcgctgcgccgcgcgagcaggaggtgggggcacccgcagcaggtggcgggggctcccgcgtgcgcaagaagggagaactcgcgccctgcagcgtctgcaggaagggggtctcgagctgcgcgtgccggtggaccctgcgcccacaggaaagggggtctcgccccgcgcgcagaggaaaggggggcacgcgccgttcgcgccagtagaagggctggtcctgcgcgcgcgcgcgcgcgcgggttggggtcaggggccggtaccccctatggctgctacgacctcccgccgcccgtggggagtagggagctgcgggctacggctgcagctcgcagctcgcggtgacctggaggggcgcggggctgagtggcgctccccctggggccagaggtccggccggggcggcggctcgaggcccagccgttcccagcgtccccgggcaacaggatcggcacgtgaggcggcgggagcccccgtgttggcacccgcggccggccgactccccagacgttgttggcgcggaaccggtggggccggaagcgcccaggagtccccgcagcggtcccgggcccacccgccattcccctgtgctcctagcagcgggcccggcatgccgggtcccgggagccctctgcgcgcgcccccgcggcgtggccaaagcgctggccgtcaggaggtgctcggaagtcgggcggtaggagaaggggtcttggtcttccacsgggactgcccgtacaccctcatgtattagccggcagtgcggcgcgcagcccaaaggcggaatggcctctgttggcgctactgtagttaccgcttagcgagtctttgctcccaaaccctgctgtcgmtttggcacaagtgtgcgacaaggacgtcagctggtatacgactgttctgcagctgggcgctggtgaatacctgatcctcgccaaggagcttcagccaagtgttgtgcaggaagaaggtagcaaggtcaatccgggagagcacagacaggacagggagtcagagggcctggcaggtcccagaggaggacgtgccaggacctgcatcacagtaggagtgmcagtgtctttaggagtcacgagcgggtttwgtattctccggttctgtggcatcttahgccgaccactggggaaagrckgcctggacaagtggacagggggaccgccccataaaggactccaagcttcagtgaccgcaggtcctccgggtagaagtatgaaacgacttctcctttgtaaaaggagtctccaggagggcgcctgtgagaagcaggctgtggtggactttggctgtggcgggtccattcaagtgcacagaactraatacaggcatgtggtggatggtcccagccttcacatcctagttgggagagtgtgctctatcattccgttcaatgttcccattgggagaggtgaggcacggtgcacaaaggaggtccctgtatcatttatttacaactacatgaaaatctacaactgtctccaaaagcaaagttgaagaagttgctggtgtaggaatgccatggcagtccagtggtgaggactccaagcctccactgtgggcggcacaggtttgatgcttggttctgaccaaggaactaagatctcatgtgctgaggagcagctaaacccgcgtatgccacagctactgaccccaggcgccacaagtagagagtccagggaccaccctaaacaatttcaagtgcaataactgacacctgacacagccaaattaaaaaatgccgaagtccaactccagtaaccagggattcaacctgaagagatgaccgtgtcagccaacaagacagcctctcagttttctatggactgcctgttcattccaagtttaagtttctcttttatacttttacaaaaacattaggctagaggtttgacattttcagttccccctctcccagatttattatctccataaatcattgtcgctgttcctgcttcagggattctctccggaatcagccttatcatctattatctacctcctctaatgtgtcctatagttaacttgtgattatattgtaactcatgccacattcctcagtttactacttatcttcctaaatcctgtttgcccctaacatcctgagctcactatctcttaaaaaggcttcttagctataatgtctctaaaaaattcctaaattctataagctatagtaaaatatgctaactttacaacattccttaaatctttaacttctattttaattatttctaagccctaaattcagtaaactcctttgccataaactttgtcttcacaaataggctttagatatcaatgcctcccatggcctcaagctacggcctgtgtgctcatcctggaacactcttttgcacaagttctttaacaaatgtcagtgattaactttatgaattattttctgagcacagctgcagaaggctttgtgccttctcatgctcctctcaagaacaataagcaccttaatattccttttcagtgaactcagccgacgagaggaaaagacaagtcagaattacaaggcctaactccttcatcctgggatccatgcctgcggaatgaggagaggagtctggggctgtgcctccattttgtcagtagtgcctaacgtggctcctgacaaaaaaaaataaaaaaattttaagtggatggatttgatcaattgaaggcctcagaggtggcacttgtgacaaagaacccgcctgccaaaacaggagatgcaagagacactaggtttcatccctaggtggggaagatcccctggagaagggcatgataatctgctctggtattcgtgcctggagaatcccatggacagaggaacctggagggttacagtgcatgggaccacaaagagtcagacctgactgagtgactaagcacatgatcaatcataaggtgaaagaccatataaatgcccctagagtttttggatgtgccctgtcccaactcggggcttcccttgtggctcagctagtaaagaattctgcagtgggggagacctgtgttcgatccctgatttgggaagatcccctggaggaaggcatgggaacccactccagtatttttgcctggagaatcccgtggacagaggaacctgtggagtacagcccataggatggcaaaaacagacagacatggctgaagcaacttaccacacatagcatcctaactaatcacctaatgccatccttctagtaggaattttctgtcttgagactataaaaatgggctgctagcccatcaaaggggtcggctctcccttgaccggccccctgttctaacagcatatcccatactgcactccgatacactctcttctcctctcattctgcctcatctctggaaacttttccarccsgtgcacrgaccaccacactcccccaaaacacacttccttcaragtgcrgtgacattcagatgccatcacatctgagaccacctgcgacctgcctccagcctctcaaaagtggacttcttcttgcttcgagatgctgagtcacaaagagtacttcggtctgtctgacccctttgcggacactcacattgacggggcggtgaacctcgtattccctgctggagcttagggagtgacctgcctggattgaagtctcagctctacgacctctgtgagcttggacacgtcacatgtcttctgctgccttggacagaaactagagatagaaaggtgagccaccaggatagggggtgccagttaagccagacttgaagttatccagtgcagccaacactgcactggaaggcagtcggataaggatactcgcgttagcttgggttccttctttgtctctggacacaaaccccaccctgtcctttccagcctgggctcgtctctttggaggctgagaaatggagctcaagacttagtcagatggytccagcattgaatgttctgctgactgctctcttaggcactatttctcaagccatccatttctgctatctcttcagaacaaagactgggtttccttcaggaagccaagtcaggtgaggttaattccccacaaaaaccacaagtcagactctttggcttctaactctcagtagctcactcatagtctgactctttgtgaccccatggcctgtagcctgccagctcctctgtccacgcaattctccaggcaagaatcctggagtggctagctgctgttcccttctccaggggatcgtcctgacctctgatggaaccctggtcgcccgcattgcaggcagattctttaccttctgagccactaagcccaggttcaaaacccacccactgtgtgcaatatttacagagctactgaattcccccggggtgggggttggggggcagaaaaagagttgcatgccaagctgtaacccacgacaacacttttttatccgcttaaagctctgtaggcaaccttgagcagttttgtccgttctggagacactgggcagaaacagaggccgaaatgcagtgacgctgttgcacagatccaccccccaaatctttggcatcagggcaaatggacaaacgcagcatttccatcttttaaggcacgttccacacacgatcttccaaaagaatgttctgctttccaggagccaaggaaatagaagatcaactgttccaaacaggtactgagatctccactctctgaaggactcagggtcttgggagtagtcctatgatggtttgtcctgtgtttacaagcaagtgcatactctccaagggtggttaaagtttcaacaccatcagtgtgtgttctgacttctatcagagcagctgcccctgcttccagacaagccagcctggatctttctgaagtggtagcgtttgcagctcctggttagaggccctgcttccatctcgcagtgacctcccctctgccttccaccccaacrgaagcccagtcctgtggttttctaggaggaaggctgaggagctgaggccaaactctgtcttatgtaaaactgcaaagctggtggtggaaatggagatgagcctctggcgtgggagacggtggaggaaaagcatagggatgccttacatctggttggaaaaattctgagacctcatgtttactgctgggagaaggtcaaagactgtctactcctggctgcaaatggacctgttaaatttcctgatggtaagtatgatggagctggtgtttttctttctttcttttttttttcaaattttgattagtgaagaatctgcctgcaatgtgggagacctgggtccgatccctgggttgggaagatcccctggagaagagaaaggctacccactccagtattctgccctagagaattctatgcatagtatagtccatggggttgcaaagagtctgacasgactgaggggctttcactttcacttttagtaaagccataacttagatgaggctcttagattttcaacttgaagactttttttttttttcaagtccactcaatatattaccaatagtactaatatcattttgaaactattatgcgtatagaattaagcatgactttcaatgtttaatgcagttcctctaaaaattaaaggagtttggcctccctggtggctaacttggtaaagaatccacgtgcaatgaaggagacctgggttcaatccctgcgtccggaagatcccctggaggagggcatggcaacccaccccagtatttttgcctggagactcccatggacggaggagcctggcgggctacagtccacggggtcgcaaagactcgaacaggaatgagcgactaatacactttaagaaaatctgaataaacttgaacttttttttttttcctaaagagagttactcttgaaaggtaacatacaatgtcaaaaatctaaccttacatggttgagatgagtaaaaattgagcggacctgaaggtataaaacgtgtgtgtgtatgtgtgtgtgtgtgtccagtttggcaaaagagtttcccctttcttcaaagttttgttgattccaagccaaagtcctagctctgtggggtgatttccctggcggaggggggccacagaaaagcagtattttcatgctaatcacggcagggtcactgtttatgaactggcaacggatcaaaatgaaggatgtcagagactcagactgctcagacctggcaagagcgcgtggtttctgggggagatgggttaagatgagacagcactgtcgagggctccgggttctggagaattaagcagccccgccccctccccctcccgggaaggcatgccgaccggcaggagagcggacttcccgaccctgagctctctgcccccacccctcctcctcgttaccaagcatcaccctcgtgaggcctcgtggcgttccgcgctgccctcgccgagcctggcgccttttctgccagcgcggggcggggatcaggcgggggcagcggggaggcccagggcacatgacgccccctccccgcggcccccgcgcccagcacatgactcaggccggcaggcagacccgagccacgcgcgtccccagcacgacccatggcctctccgcgactaggcaccttctgctgccccacgcgggacgccgccacgcagctcgcgctgggcttccagccgcgggctttccacgcgctgtgtctgggtagcggcgcgctccgcctggcgctcggcctcctgcagctgcggcccgggcgccggcccgcgggccccgggatcgcctcagcctcgccggcgacctcggcccgcgtccccgcctccgtgcgcatcgtgcgcgccgcaaccgcttgcgacctgcttggctgcctgggtgagcgcgcgagccgcgcgggcgagggtgggtggggaacccgtcggtgttcgagtgagggcgcccggtccgcgggtggggaccccttcttgggaggtgagggcgcacggccggggacccctccgtgcgtggttcagggcccgtgcgtactgcactgatggagacccctccgcgcgcagttgagtgcgcgcggcccgcgagtagggctccctctgcgggtgaagacgttgggcccctaagtaagggcgcgtctatgcgttttttcagggcgtgcctaggtgcgcggttgagaacccgcggcccacgggtggtcctccgtgggagtttgacggggtgaggcccgccagtggggaccccttcatgggagttgtgggcgcacggcccgtcgtaggtggggaccactgagtgcgcggttgagggcgcgcagctcaaaggtggggacccctccgtgcgcagctgagcgcgagcggcccgcgagtagggctccctcagtttgtgggtgaggacgcccggcccccaagcagagacccgtctgtgcgttttttcagggctcgcctacgtgcgcggttgagagccccgcggccccacgggtgggcctccgagcgaggttgacgaggtgtggcccgccactggggactccttcgtgggagttgggggcgcacgacccgtcgtaggtggggaccactgcctgcgcggttgaggacgcgcggctctacggtggagacccctccgtgtgaaggtgaggacgcctggcctgcgagtagggaccgtctgtgcgtttcctagggcacacgcaccgcgggtgcggacccctccgtgagcagttgagggcccgcggcccgcgagtggggatctttccgtgtgtgcttgaagacgcgctgcccgcgagtggggaccccttcgtgcgcggttgagggtgcacggcactgtgtgtgcagttgaaggcacacggcccggggtgggaacccctccatgcccatttgaaggcccatggcccacgagtggggacctctctgtgcccaatttagagcgcgcggcccgcgagtggggacgcccttcgtgggaattgagggcgcacagccttgcggtgacaaccactccgtgcgcggttgagggcacatggcctgccggtggggtcccctccatgtgcaggtgaggacgcccagcccacaaggagggacccatctgtgcgtttttcagggaacgagtaccgcgagtcgggacctctccgttcccagttgagggtgcgtttccggtgagtggggacctctctgtacgcggttaagggcgcacaccccgcgggtgcagacccctctgtgcgcggttgagggcgcacagcccattggtggggaccactgtgtgtgcagttgagggcccgtggatgcgggtggggaccattgtgtgggcagttgagtgtgcacggcccgcgggtggggtcccctccgtgtgtgggtgaggacaccaggtccacgagtagggacccatctgtgcattttccaggggtcgattaccatgggtcgggacccctccgtgcccagttgaaggccaatagccagagagagtggagtcccctgcgtgtgcggttgagggcgcacggcctgcaggtggggaccactgcctgcgcggttgaaggcgccagtccgcggatggaaacctctctgtgcccagctgagggcgaatggcctgctagtggggacctctctgtgcccagttgagaacgtgtggcccgcgagtggggatgcccttcataggagttgagggcacacagccttgtggtgaggactaccactctgtgcgtggttgagggcacacggcccacgggtggggagcccttggtgctcagttgagggcacgtgtactgcggtggggacccctccatgcccagttgagggcccaggagtggggacccctttgtgtgcagttgagggcgcacggcctgcgggtggggtcccttccatgtgcgggtgaggacgcccagcccatgaggccccttccgtgtgtgctgagggcacgctgcacgctagtggggaccgctccctgcgcagttgagggtgcatggaccgcgggtgggaaccactgtgtgagcagttgaaaacacacgacccaggggtctgcgagtggggacctctctgtgcccagttgagagctcgtggcccatgagtgggtccttcatgggagttgagagcgcatagccttgtggtgaggaccactccgtgcgcagttgagggtgcacagcccacaggtggggaactcttggtgtgcagtcgagggcgcgcctacagccttgaggacccatccatgtgcggttgatggtgcgtggcccgtgagtgggaaccctctctgtgtgcttgagggcgcactgtctgcgagtgggaaccactgcgtgggcggttgaaggcgcacggcccgtgggtgggaagccctccgtgtgcaggtgaggatgtcgggccgcgagttgggacccgtctgtgcgttttccagggcgcacgtagtgcgtgtcgggacccctccatgcccagttgaggcccgcggccctcgagtggggatacctccatgtgtgtttgagggtgcactgcccgcgagtggggacgcctctgtccgcggttaagggcgcacagcccaggggtggggaccactgcgtgcgcggttgaggacacctgccctgcgagcagggagctgtctgtgcgtttttcagggcatgcgcacctcgggtcagggctcctctgtgtgctgttgagggtgtgcagccagcgaatggggacctctccttatgcggttgagggcacacggcccgcgatgcgaacccctccatgcccagtggaggccgcggggcccgagagtggggacccccttcctgggagctgagggaacacggccctcgattggggaagccttcgtgtgcagggaaggacacccgtcccgggagtcgtgtgcatggttcagggtgtacgtaccacggatggggaccactccttgcatggttgagcgcacctggtctccgggactgggggaccctccatatgcgggtgaggacgccaggcccgcgacccgtttgtgagtggttgagggtgcccggcaggcgggtgaggtcccctccgtgcaaggatgagggcaccccgcccacaggtggggacccgtctgtgcgtggttgaaggcatgcggcccgcaagtggaaagccctcggtatgagtgggggtgcccagcagtcgggtgrtgcatgttttagggcctgtggcccatgagtggggacccctacgtgtgcaaatgaggatgcctgccccagggagcgggtaccgcttgtgcgtggttgagagcgcctggcctgcgggtgggaggactgatccagagagggctacccagggacactacgaggacgagtggattctgcaatacagtatgggtaaaagttgcttccggagacttctttttttcttttcttttctttctttctttttttttttttaacactttgatgtatttgttactgctcttactagggaaactttaggcagtgctgtgaaggaaaacacagaaaataccacggttccatgatcgttccaccaatgagagactaatagttaatattatcaagcctgtgctcacatacatgcagttgtttccaaaccgatttagcagaatcaggaagaaaactccatatatacgataaaatttttatttccttttttatctatgtatctaaatttgggttttctatcacaaatgtgcaaacttgtagtctctaggctaaatccagcctgcttttctatgtacagcccatgagttaagaatgtttttacatgttaaagtgtgttttaaaaatctaccagggggcttccctggtggctcagacggtaaagcatctgcctgcaaggtgggagacccgggttcaatccctggatcgggaagatcccctggagaaggaaatggcaacccactccagtactcttgcctagaaaattccatggatagaggagccctgtgggctacagtctatggggtcccaaagagtcagacacgactaagcaacttcactttctccctttctaagaggagagtgaaaaagctagcttaacactcgaagcctagaaatctacagaacagggacttccctggtcgtccagtgactcaaacactgtgctcccaatgcaggagacctgggttcgatccctagtcagggaacaggatctcacattgcggcaacagagacctggcccagccaagtaaataaatacttttaagaacatctacagaacaatggtatttcagacatgtaagcattctgtgaaatgtgaatgtcagcgtccgtaagtggactcttatggggatacagccgtgccctgcacttgtgcactgttggggcaacatcttccacccaccccatttccccatcagggccgaaatgagtgttgcatagagaaacatagagaaagtgtgggccacagaccctaaaatattttctctctgatccactacagaaactttgctgacccctgctttcaaccctagcacggcacgttttacaaactctaagctcgatactgctgaccttttaaacttttttattttggactttattatatatatgcaaatgtagagactgtataagaagccgcctgttctcatcacctagcctcctctatacttcatccctgtaccccactccccactgaattaatttgaagcagatcccagaatttatatcattacatccagacaaatctcagtatgtaactgtagaagaattcaacatatatataattatatacataattataatatataacatgtccattgtaacatactgtatactacatgtgtgtgtatgttagtcactcgctcatgtctgactctttgcagccccatggactgtagcccgccaggctcctctatccgtgggattctccaggcagtaatactggagtgggttgccatttcctcctccaggggatgttcccaacccagggatcaaacctgggtctcccacactgcaggtggattctttactatctgagccaccagggaagccatatactatatgttacaatattataatatattacaatatataatatatttaatatgttatattagtgaagtgaaatcgctcagtcatgtccaactctttgcgaccccatggactgtagcctaccaggctcctccgtccatggaattttccagacaagtgtactggagtgggtttccatttccttctccagggggtcttcccgacccagggatcgaacccgggtctcctgcatcacaggcagacactttaccctctgagccatcagggaagcccaatatgttatattaagtgtatatttaatatagtctcatattataatatgtaattatatatatatatatatcacactgatatcattgccacctaaaaaataaggaaaaccgattccttcatgtcaccgctacatgttcaggcatgtcatacatgccagttttccgttcgttagttcaaatcaggatccaaagagcgtccgtgttctgtgagtgctgacgggtgctggccatgttggaatcgtctgctcttgaaggagaggctctgatggtatttgaatggcttccacctccgtgtcctgagggtgtcgcagcagcttctagcccaccttgaccagatacctggtggatgtgttccaggggttggctcccctgttcctcttgctgttttgtgcttggagacactcaagaatcttgactccagacacatgctatggaacttgcagtcactttttcagactggagtaagacaggaaggaatacctccctttctgttagtgacaaagcgagtgtggtgcaggataattaaattttaaaaaattggttttctccctgaytctgctgaagcatttgggggaagcatcttaatagtttcagtaacttgaatatctggagatagagttgcagccaaacatcttttggggggtgggtcaggaaataagattggtacatcctttcaccttttgaaggaaggtacatggcactgtgggcaaatgagcagaagcaggagtcagggcaaggcagggggtgtctggacctcctcgctgaaaaataacaagcacagatgccaaggtgtccctggtggtgcagtggtaaaggacccgcctgccaatgcaggggacatgggttcgattcccggtccaggagggtcccgcgtactccagagtctgtgagccacaactgctgagcctatgctgcagagactggggtccataacatctgaagcccacgacccacctagagcctgcgctccgcaacaagagatgccacggcgatgagaagtccacataccacaatgaagagtagcccccactctgaaactagagaaagcctgcaaagaccaagtgcagccaaaataaagacagaaagaaatacagatgcctattgcatggaatgtgattctgctcccccactaccccaccccccccggaggattttttaaaatcttttttttttttcctggacaaattgcacagcttgtgggatcccagttccctgaccagggattgaacctgggccatggaagtgaaaccacagaatcctaaccactagaccaccagggaagtcccgccccagttttgctgacacttcactgacatacagcactgggaaacgtttacatatccagcacaatgactcacatttaccataaaatgtttcctaaacacccatcacctcatatacatgcaaatatatatacatatggtgtttactgagctcaaagcagatctccatagtgaaaacaacaaagcactatttggaagaaaacgtcatttctgttatttgcacttaagaataagaagcgacatctttaggattttagtgtccataatgccctttggtggtgtaggacacagtgtatatctggttgcctattttgaagtatatgttttgtatgccaactacacacattatgctagtatacaagtctgtactgttcatttccccttgagcattttgtgttatacactgtacactcaaacagatgctatcccaaacctcacaaccctattatatacgttgagtgtggatactgattataggcgaattagatatctggatctaagagcaaggaacactcccaaataataaatgtcaaaagtagattacaagagctaacttgtgaaaatcttcaatttttaattttattaaaaagaacagaagaactctctttcacaacttcagaataagctttttcgggacttccctggtggtccagtggttgagaatccaccttgcagtgcagggcacacgagttccatccctggtcagggaagatcccacatggcgtggagcgactaagcctatgcaccacaagttcggagcccacgagccatgactactgagcccaggtgccgaaactactggagcccaggtgccctggagctggttctccgcagcaagggaagccacagagatgagcagcccaagagtagaggcccctgctcgcggcacctagacaaagtcctgttagcagcaacaaagaccaatcaataaaataaatcaatacatccttttttaaaaaaattaaaaaagaaagaatgtgctttttttcagtcgttcttttttttttttttaacatgaggactagttaagccggacactaaaaatacagccaagaaacacacactgttattcacacagttgcttcttagaggcaaataaacgaccaaccaaaataaggaacatgactctagctgatgttagtggaagtggaaatcaaagcgccggggattgctctgcaaccataatccttatcttctgagtctttttttttttctcctctgtctctcctgtacccaccaacatgtgtatctttcatctctgcctccctcacatctttctctctcccttcctatcttctatccatccatcctttcatctgtccatgtgtccatttgtccatccatcatttttacagtcagcagaccccctttaggtctgagtcaggagcggacacaaagaaagcgttatcacctggttgccccttaggtatcgcggtccgatctgcggtgtggttagggtttccgagtttcgtggacgacatctctgccgtgaacaacacagatgtgtggcctgccgtcttctgcgtggggagtgcagtaagtgtggcttccctggtcaccctccccaccccttcactcccctctgttacactgagtcctcaaagtatggtcctgaccctcccaccaacccagtgaggtttttagaaaagctcagagacccccacccttagcccactgaccagggaccctgtgggaggaaggcctgtctgtgatttaacaaaccctccaggggtctctggagcttcccaggtggctcacaggtgaagaacccgcctgctaatgcaggaggtgctggtttgatccctgggtagggaagatctcctggaggaaatggcaacccactccagtattttttgcctggaggattccatggacagaggaccctggggggctacagtccacagggtcgcaaagaatcgaacaccattgaaggcacttagcattgcagtacatccctgccttgctccccctgggaaggtccttcctgcgcatcctctttcgtcctcagcagaagcgcatctggttcaaagtgcctggccaggcctttcatctctgagcccgtcatctctctgcctacacatgtgagcatcaggaggggcttgctggaccagtctggctgagcagaccgggaaccaaggcacccgcctgcccccaggccacgtcccagcctaggcttccaagggtggccacgtgtctgccgagggctttcagggacacgagatgacatcaggggtggggcggggccacctccagcctgcacctaggggctgggatgccattcttctgattatagctctggatccagctgctgtacagtgcctgcttctggtggtggttctgctacgcagtggatgcctacctggtgatccagaggtcggctggacagaggtattcagtgcgagcggccgggttcttctgctctagctcattgccggaggggaaggttggagaggccacgagcacttatttctgtagggtagacacggaaagtgaaagaggagagcgaaaaagttggcttaaagctcaacattcagaaaactgagatcatggcatccggtcccatcacttcatggcaaatagatgggaaaacagtggtagactttttttggggggggctccaaaatcactgcagatggtgactgcagccatgaaattaaaagatgcttacttcttgggagaaaagttatgaccaacctagagagcatattaaaaagcagagacgttactttgccaacaaaggtctgtctagtgaaggctgtagttttcccagtagtcatgtatggatgtgagagttggactataaagaaagctgagtgccaaggaattgatgcttttgaactgtggtgttggagaagactcctgagagtcccttggacatcagggagatccaaccagtccatcctaaaggagatcagtcctgaatattcattggaagaactgatgttgaagctgaaactccagtcctttgggcacctgatgcaaagagctgactcattggaaaagaccctgatactgggaaagattgaaggcaggaggagaaggggacgatagaggatgagatggttggacggcatcaccgactcaatggacatgagtttgagcaagcgggagttggtgatggacagggaggcctggcatgctgcagtccatgggatctcagagttggacacgactgagtgactgaactgaactgagacacagtccatcagaacctcccagtgggcactccaccaagaaatgtctaatgccagctcgtttttatgtaaaaggcagaaggcacaatcaaaacagcaaggtattcttgctgggcacctgaaacattgtaagtcaactatatatatatatgttatattatatatatatatatatattaagaaaaaatagcaaaatattctccaggtgcccccccaaccaaaattgaggattttaaaaccatggaaataagtaattcagtaaatttcgtatttggaactggacagatgctaaaaaaggattggcatgattataaaaattctgatggtatctaaaggtataggaagaagttctaagaagtgtgtatggatgtgttatgctaagtacagagcccaagatttctggtgctggagagggggctgtctaggaggaattacacagtggttagtgccttggccccagtgggttcaagttcctgaccctcactcactcacctcagtcactgagtgactgttggcaagtttctgaacctctctggatcacctgcaaggcttactgactctgctcctttggggggacagggtggcgtgtgtgggtgtacagagatgaaggatggggatggttaaggatgtaaggatggaggtggtaagagtgtaaactctgcaaggaatggtcaagggtggcagtggagagagataggaagtctctcagctctcctggaatgtcagatggccacccacttctcagagaccttctttgtggtttggactgagcattaagacccttgcatgttgggtacccagatgacttcaggaaggacagaggctgggaacctcttgctactgggggagctaggtccctaggaggaattatgccagctcatttttataaaaggaagaaggcacaatcaaaacagcaaggtattcttgctgagcacctgaaatgtccatcccttctgtcaatcttgagagccttcagttcagttcagtcgctcagttgtgtccgactctttgtgacccctgcagcacgccaggcctccgtgtccatcaccaactcccggagttgactcaaactcatgtccattgagtcagtgatgccatccagccatctcatcctctgttgtccccttctcctcctgccttcaatctttcccagcatcagggtcttttccaatgagtcagctcttcgcatcaggtggccaaaggattggagtttcagcttcagcttcagtccttccaaaggatattcaagactgatttcttttaggatggactggttggttctcctggcagtccaagggactctcaagagtcttctccaatgccacagttccaaagcatcaattcttcagtgctcagctttctttatggtccgacttgcacatccatacataactactggaaacaccatagctttgcctagatggaccttgaaagccttaggttaagggaaacaaaccagacacagaaggccacattttttacacttccacttggatgaaatgtccagaataggcgaatccatagagagagatggtagctgagtggttgccaggggctgggggtgcaggcgtgatggggggagacccccgaatgggtgtgggtgtccttttatggggatgaacatgttttcaaacgggcctgaggtggggcttgcatggcactgtggatatcctaaatgccgctgagttgttcactttagaatggtgactttgatgttacatgactttcactactcagcactaaggggtgacaatacatacacaagtgggcacagctgtgtgccaataaaactttatttacaaaagcaggcagagggccggatctgtacggagggctgtgtttaaacaaataaacaaacaaaacaacctcagaggcacaatcagggggctacttgctgagattctgctctgcatgtgttttaccgctgctcgcagagtgtggggccgggcaggcgctggacctgaggttctggacctgtattctctctcccccagcaccatcctgctgtaccacctcatgacctggggcctggctgccctgctgagcgtggagggtgccctcatgctgtactatccttccatggccaggtaagccggggctcccccaatgcctgacccccgtggggcgtcctcctgcagacatgcctgggtgctgaccccatgtgggtgggcgggccttcgtccaggatccgtgcagctgcgagcacgtgcctggctccttgagccagccagggtcccctgctcctgcccatcacgtgtgttgggttggtgctgtgcccagtgtggatacagagcttgcagggcaccatgggtgtcctaaatgctgctgagttgttcactttacaatggtgactttgatgttatgtgactttccctactcagcactaatgggtaaagcagccatagacaatacatacacaagtgggcatggctgtgtgccaataaaactttattgacaaaaacaggcggagggtgggatctgtactgagggctgtgtttaaacaaacaaacaaaaaaaacaacctcagaggcacaatcggagctgggggtgcatctctcctccctacacacctgtgatggctctctcctacctgctgttgatttttttttaatccttcaaaccagctttctctgggcacgcaggtgagttgaacatcccagggctgagttccagatgtctgggagactcaaagggttgcttttagaatagctcattttcagcctctgctgtctgcacttttttttcatgtctccatcctgtgctcttctgaacacacgctgggagatgtggcctctgaaactgtctcagacatttctttgagtctgaacccctgccctttaccatttgtaaaacctcaaggtctctctaaggagatatattctacattttaatggaaaactgaaaatcagacgttggacaccatctcttggtgattttaaggtgttgtagacccagggaggggcttcctgggtagctcagctggtaaagagtctgcgtgcaatgcaggagactccagttcaattcctgggttgggaagttcccccagagaagggataggctacccactccagtattcttggacttccctggtggttcagacagtaaagaatccgcctgcagtgtgggagacctgggttcaattcctgggttgggaagatcccctggaggagggcatggcaacccactccagtattcttgcctggagaatcccatggacagaggagcctggcgggccacagtccatggggttgcaaagagttggacacaaatgagcaactaagcacagcacagcacagacccaaggatcagtacagcatggcccatagtacagatccggccctttgcctgcttttgtaaataaagttttattggcacacagccatacccatttgtgtatgtattgtctatggctgctttatccattagagttgagtagatttgacagaggccatgagggttctgaagcctaaaatatttcctatctgttcctttagagaaaaagtttgctgacctgtggtgtagactgtttctccccatctttcttaatttcttgcagctgctcactcaaatgtctccacagtcctattgctttgtcaactgtcactacttccaggcctctgtgtagacccctcaaaatactctgctctccccaggagccttaaaaggacgaagacagagtaagcagctgttacagagggatgcacttaatcggaaagagaatttttatcactcacctccacccatgggatcctggggctgttttcatttgagcccaggtgctcccagattaacacaaggcctccacctgaagcattttcattctccatcatcttatcctccaacatctccaccctcaatcatccccaccctctatcatctccaatatgcatcacctccaacctccatcatctccatccaccatcatcccatcatgcccatcttccatcatctcataggccatcatctcaccctcgaacatctgcacccaatatcatccccaccctccaatatctccaccctcaatcatccccaccctccaacttctgcaacatcacatgcaaccatcatcatctgcaacttccatcatcctatcttctctaacttccatcatctcatcctctatcatctcatcatctcgtcttccatcatctctactctccatcatctccaccttccatcatctcatctttcatcatctccaccatccatcatcccaccctccatcatctccaccatccatcattttcatcctccatcagaaccgtcctccataatctccagccaccatcatctcatctgcctcatgtccaccctccaccatctccaccctccatcagtctgccctccaccatcccatcatctccaccctccatcatcccatcatgtctacctccatctcctcctcctccatcatctccttctccatcattgccacattccatcatctccaccttccatcatcccatcatgcccaccctgcatcatctcatcctccagtgtcttcaccctccatcatctccaccctccatcatcccatcatcttctctgcaccttccattgtctccacccttcatcatttgcatcctcagagcactgtgcaggctcccaccctcactcatctgtccctcatcattcgaatctcatcttttgctcctcctgtttccctttctcccctcaagcccttgtagaccatggtttagctttcacagctgaagaagaaagcttcatttataaaagaacgcaccatcttcacatggtattgccatccttagggcaacaaaaggtgggcactgcattgttcatgcctggttttgtgcattcttccactttcatctctggtgtgtgcactccatactgatgacagcaccagcatctttatcatgcccccttcccgggcctccagggctacgccctcctctggctttccccctcatctggggttggacctgagcaacttctcctttggatgccctctccccctttgccactttctctacattatgcatttgttcagtgacactcccggccttggtaacatagctgtgtgtagcataccaccatgtcccgttgctgacggacgagtcccccagcccatcagtagacattcctgtctttgtttctgaaaagaaccaccgaaatgattatgggtcaccttgggaacaagtgtcctgagaaataggaagctcgggtgttttcagttgttcgagggcagagcttggatcttattggtgatgtcagccaggcctagaacgtagtaaagtgctgagcaccttaccagtctattgagtgcctttgtcttgcttgaactgtgtgtgttttctttgaaggggtcccacaggggggttgtggtgggggtgggcgtccatcatcccagagtctttcctgtctttacttttttccaagggggaacttgaaaagtggcccactttctgagacaagacagaactgtgattgatcttccttctccttctgtgacttacttgcttgtccacagacatgatgagatgtgagtcaggtttccttccggcagccagaagtgatctgtgcatttccctttgcgctctgggtcctcttatctcggctttcagataaggcagcgggttatttcaggtaaagccttattgttggagaatgcctcggtttttctctccccccctccccaccaggtgcgagaggggcctggagcatgccatcccccactacatcaccacgtacttgccgctgctactggtcctggtgggcaaccccatcctatttcgaaagacagtgaccgcaggtaaagggcagggaagcttcctggggatgaggccacggggatgagccccaggacttgggccccagtcattacctaggatccctgacctcatctgtcatcctgcagagacgagcagaatgaagttggggagagtgagtgggtttacattagtgacagggagcaagcctactcagtcacggctagatggtctttgccagcatgcgttagcgcacagcccagggacctgtggactttttcccttctacctggactcatcctgggtctatccgcggcgtgcccccaaagacccagtgtccttgaaaactggcggagggggagatgggaacaggagaggaaaggaaaggtcagccgtgggcgaggggtggagatggacagggctgtccccggtggcggaggggctggctccccccgtgctgtctgggcccatgtcttctcccagctgggcaggaagcaggaaggggcccacaggacatgagtcagaggcaccagaaagatctgggcgagaaggtgagtcagcagacccacaggagggggcctgcattccagccccctaccccatccttgaggagacgagccctggggcaattctctagccccctcgtgccctgtgggtccctcacgggcagaattggaataaagaaacacacctccacagaaaggtcactggaggatcgactgtggattgcagaactttaagcgacatgctctgctacttagggagacaacatcacgaaggctctttgggtctctctctcttcgtatctgcgttgaaaaacagtgtgtgtacacctgtcagtaacacaacattggaaatcgactctactgcaataaaaatttaaaaacacgcaacccccatagtgtattagaaagaaagaaagtgaagtcactcagtcgtgtcagactctttgtgaccccatgggttgtagcctaccaggcttctccatccatggaattttctaggcaagagtaccgtagtgggttgctatttccttttccaggggatcttcccaacccagggattgaaccctggtctcccgcattttaggcagacgcttttaccatctgagccaaatacagctttttaaaaaacattgcattaaaaacagtgatccaattacatcaaataaaggaaaacgaagtgtaggaaaaaacactggaaagaaatatactaagatgctaacagtagttgtctttggggggatgataggttattaattttttttctaaacttttttttatatttttcaacttgtctaaaggaggatgccaaatctttcaatattttaaaatagcaagtatgagactcccctggaggtccaagggttaggactacacgctttcactgcaggggggcctgggtttaatccctagttggggaactaagatcccacgtgcagagagtcttggccaaagaaagaaagaaatcaaggataaattattactgtttacattttttggccacatctttcagcatggggcattatagtttcctgatcagagattgaaccttgccccctgtgttggcatggaatcttataaccactgaaccacgagagaagtcccctagtgaattatttttataataaaaataacatttatatttatatttaatatttataaaataatttttattataaagataaaaagtataaactgttaaaaatacagctcctgaacgaataaaatccagataaaatcaggggtgtagttaacagcactgcaccagcgttattttcttcatgctgaccaacgtgtggctgtgagtgtggttgcggaagacgttaacactggaggcagctgggtgcagctatatgggggctctactatctctgtatatcttatataaatctaaacaatttcaaaaatgaaagtgaaagtggctcagtcatgtcgtgaacccatggactataaagtccgaggaattctccaggccagaatactggagtgggtagcctttcccttctccagggggatcttcccaagcccagggaccgaacccatgtctaccgcattgcgggcaaatttaccagctgagccaccagagaagccccagaatcctggagtgggtagcctcttccttctccaggggctcttcctgacccaggaatcgaactggggtctcctgcattgcaggcggattctttaccagctgagctatcagggaagccacaaagacttcaaaggaaacctttaagagctgtgtattccccccgagtgtctgagaagcacccccgcccctccccatgcagtggtgagagaggcatggctggtatcttgcctcctgctctccagggtgacttgcgggctcccatccggtttgctggcctttgtcgatcgtgaacatcacacaccgttcccctctgctccacagtggcctccttactgaagggaagacaaggcatttacacggagaacgagagacgcatgggagccaggatcaagacccgattcttcaaaataatgctggttttcattgtttggtaacgtgtttctgtttctttaaactctcactgggacgtgaagtgcaaacctcttgtaagttgccttcccaagtgtgacctgagttccagaggcagagtcttgggagaggaaacctttagaggcttgtccagatggccctggaggcaatgctgtccagacaggaaggccagtctgcacccatgaccttctggaaagaaattcacatccgctcaaattatagagaatcgctaagaacagaaaatcctcactttaacgtggtcacatcccaaccacttgcctctggagtactgtcccatcgccgagggttaacattcaaatcaaatgcttcatcgtgccttctaaatccatcttttatttttgcacccatctttgttttattttttaattaattagttttttgttgttgttgctgcagtgtgaggcatgtgggatcttacttccccggccagggattgaaccctcatccctcttgcactggaagtaccgagtcctgaccactggatggccagagaagtccctgcatttgtcttgacagcaggattcctgtatctgatgagactcagggatgacaccccgtgtctgtgtgatgactcacagctcagggcaacaaggacagaaaccatgactccccaggcagtccagtggttaggactctaagctcccactgcagggggcaagagttctatctctggttcaggaaactaagatcccgaaagctgtgtggcacggccaaaaaaacaactaaaaaaaaaaaaaacaaaaaaacaagaagggtagagagcaatcttcatctcctcagtgggcctgccctgtcccccacaggcagtctgccccagcatttcacgtgggtttgctctgtgcaaatagagctgtgacaagtaccaaatcagaaagccctttgctgcagcgacagagtgctttgccagacagggcacacgttggtggttaataatttatctgatgggaagaggctggtttggacattctcctgggagagccgtgagtcacctccctttaaaaataccccgagcctgccatttccaacttcgccgacctgccgacactaccgtcagaaatttccgacataaaacatcaaggttcgtgttggtttccatcaagacgtcagcttgctcgtcaaaggaggaacgacttctgggctatttgacgtgtgccagaaccttccacccgatgtggtgttgattgtgaccatcgggagggtctcttggtctctgcttttggctgagggggatgccgtctctgacgtctaattctcgtctaattctgcagctacttggtcacccctcccggacttctaggtcttgggagacatgttccctggggcttctcaggtggctcagtagataaaaaaatctgcctgcaatgccagagatgtgggtttgatccctaagttgggaagatcccctggaggaggaaatggcaacccactccagtattcttgcctgggaaatcccatggacagaggagcctggagagctacagtccattggggtcacaaagagttagacatgatggagtgactcagcagtcatgcatgcagacatgtcctctgacctctggcattgaaaaaaaaggatattgggaagcaattatcctcccattaaaaataagcaaaaaaaaaaaagaatgatatcacatgttctctttacctgtggcccattttctttctcacagctggttctcaaatgtcatcaacgaaagccttttgttctatcttgaaatgcaaccagatatcaacagcagctctttgaaacaggtcagaaacgcagccaagaccacgtggttcatgatggtaggtcaatcttgattttaaatgtacttcctaaaggagaagaaaaaaacccagacaagataaaaaccactgagaagtatgatctatgtgttatttaaatatgtggtgagttgaagaggttgcgggggcccccttatctggctgtggtccatgagttagcttgtgcatttactcttggcatcagtattaaagggatccgactctggtaagtcagtttggtaagtgtctcaaacctttcaggctgactaaacatatctgggaatttatctgcaagaaatgtttcagactgaaaaagccacaaagttggaggtattccttggaacactatccataaatataaagcattctagacaacccacattttcttttttttataaaagggaatggttaagtaaatcactgtgtttgatcttgatggaatggtatgcagatgctgaaaatgacaatgtaaagtatgacaattatgaaatctcataataagtgaaaaatgcaggatgtagagattatactcatttcaactacctaatcacacatgtaggcaacaatattgcctagaaacacaccaccataatcaggtggaaagctcatggatacatcgtcctcaatgtttccaggatattgtgtccccactgcaaaaactccatacaggagctgttagtggagaagtgcctgggtttccgtggtggctcagcggtaaagaatccgcctgccaatgaaggagaccccagttcaatccctgggtcaggaagatcccctggagaaaggcctggcaacccaccacttagcaagtaaacaagtggagaggctcaccgtgtcctcaattagatgcgtgtgcattcagtgttgccatcattattaaacacatatgactctggtaagttagtttggcaagtgtctcaaactttttaggcagactaagaatatctgggaatttctctggaagaagtgttgcagactgcaaaagccacagagttggaggtatcccttggaacactgtccataaatataaagcattctagagaacccacattgctggggtgatgcccgggaccttcctctccacccctcttgtctcctatgtctcccacacatctctgaaggctcagaacctccaagccagtctctctcttgccccaaactagccaacagggtgcaccccagttgaggccagccctgtggaccgaagtgacccctgtaagccgtgatgcccatcagtgccctgacttatatgccgtctctttcccctcccttccaggggatcctgaatccagcccaaggtttcctgttgtccctggccttctatggctggacgggctgccgcctgacgcttccaggtcccagcaaggagatccagtgggactcgatgaccacctcggccaccgagggggcgcccccctcccccgggggcccccaagagcccggggaaggccccgctcccaagaaggagcttccgggcggcacgcacacttccgatgaggccttgagcttgctttctgaaggtaagagcctctgggtagaggcaggcctaggttgggggccccacgctccccaggaactcctttgtcaatgaccaggaggctccctgccctgatttctggaaccagcttcagggcatgagtggaaagggaccaccttaatcttgcagctcatcactcacctctccttgtaatgtgagcgtggtgcctttcctctgcctgcgtgccacgtggcttcagtcctgttgaactctctgtgtccccagggactgtcaccgcggctaggctcctctgtccctggggattctccaggctagaatactggagcgggttgccatgccctcctccaggggatcttccccacccagggactgagcaccgtagccttgtgttaataatcactagctctgccttgggctgcatgacattctgagtctgagataaacggtcacttctcatccggtgctgacgtgtgctgtaagacaatgaacagtaggcgacctgacctctggagatttttacgtgcagttttggtttgtccacttcatccgggggcatttaagatgaaatgcaggtcacccgtcgctggctcttccactctgtcctcttcaagactctcgcaccgtggtcctcaaattggtcctctttttaaactcctgttcatctaatttggacttcccctggtggctcagatggtaaagagtctgcctacaatgctggagacctgggttcgatccctgagtcaggaagattcccctggagaaggaaacggcaacccattccaaccttctgcctggagaatctcatggacggaggagtctggtgggctacagtccgtggggtcgcagagtcggacaccactgagagacttcactttcacttttcatctaatctggggttatgtccattaagccaactgatgtggagggtgcaagtattagtttaaggcccaaataatgtttctttaaagcttgaaagtgaaagtgaagtcaagactgctttcctttaggatggactggttggatctccttgcagtccaagggactctcaagggtcttctctaacaccacagttaaagatgtgggtgaaaatcagcaaagagaatgactccaatatacaagacccctatgtctataccacaggctgtatggtgaggccagaaccctcaggaaccgggggccttttcgaataaagtggtaatctcggaaatgagataaggctgccttaatctcgggcgtgtttcctttctcctttccagccactttgctttcataagacagaaaatactgttacatgtcagagaaaaacccagagatacaaactcgaagtggtaagtacggttaggcctctccccgtctgcactgttaggatgaagcttgacctgtttatcatatccttaagcctctatctgctactttaggattgacttccaggctgacaggcttccaccgagccttcgtcattaagcatctaacatggtgtctggcacttactcgccacttaacagaaaaggcaactgaatttgaacaaatattccttgagcacctgcaacccaatcaacactactttagctgaggcgggggctggggtgtgagcgtctctgagaagcataatgtgggcaacttagaaataaagacttataaataaataaaggctgaatttgcacgtagtttggtggtgggaagctggagagaggcatgtggggaagagatctatccaggggtgtttacagattctgcatttttatcttggggccagcaaaaggccagttaaggtttctgagcggagatagaacacttacatttgctcgtaggactaacctagtgactggaaaattccatggatggaggagcctagtggggctacagtccatgggttcacaaagagtcagacataactgagcgacttcactttcatgactattgcggcggctcacagtgagaggatctgtgtataattcctgatgacaacttgagttcaaaatacgaggagcatctttgtaaagaatcaggatttcccccaagccctcacatctttccaattatcccttgcttttccagtcttcctccttctccatgcaatcccgtgtttcccacaaagctctatttccctgtaattccctacttctctctccaaccccatgtcttttccaactctacacttctcccacaattttctttcttccccataatccttgccctgcccacaactctccattttcccacagctttctgtttcattccagaattcctcttttgaataattttactttcccacagctctttgctgtctgtggtcagtctcatatcaccattttatctcctagggttctgttcccgaccacgtttcagttcttctgccattttattcttctatgattttctgttctcaattttgcttttttcttttccacaactgcctattcatccccaaattattcattcttatgcaaaaatctcccagggctcccctaaccctctttttcatgacgaccagtctctcccacaacacgctttccccgtaacgaaacaacgtaagactgtatgatccttgcccactgccgcccgccattccgtcatgttctctgcctgcctgctgtgggtggagtttagtcaaacaatcagtttaatcacagaggtgagaaatgctaaaacaaaggaaaacagtcaaaggagaccaaataataataatgtactcattaagtgtagtgagatttccactgtagctcaaacagcaaagaatctgcctgtgaggcaggaaacccgggtttgatccctgggtcgggaagatcccctggagaacagaatggcaacccactccagtattattgcctggagaattccatggacagaggagcctggtgggctacagtccatggggtcacaaagagacatgactgagtgacgaacacacagacaccaggacctttagttctttctgaaggggctatagataatattctgagccacctcctgtgagctgtcttatagataccagaacacaccaggtggaaattaacgacatgatgacccaactgtacccaggacttgctgctgctgctgctgctgctgtcgcttcagtcgtgtccgactctgtgcgaccccatagacggcagcccaccatgctcccctgtccctgggattctccaggcaagaacactggagtgggttgccatttcctcccacaatgcatgaaagtgaaaagtgaaagtgaagtcgctcagtcgtgtctgactcttagcaccatgaagtcagtgaagaggattatcagaggtgactgtactgcttctgcgtgtaacgcgcctcccccaccccaccgcaactttgtctctaaaagctctcactctctgcctgtcgggggggttggcctttggacagacgtctgccaccctccttgtagttgctggcatctgaaacaaagcaaatttttcctttccaccaacctggcccgcttattggcttttgagcggcaagcagccagaccctttcatgcataccttttggtcactgtaatcttctgttctgacagtcctgtatctctccctcaagttcctgtcctttcccacagcaccctttctcacgattctgtctctcccatctctctgtatcctctgacacaattctgtggctccaaaagttcctttttctctccgggatgccgcgctttctcacgggtctcttttcccttcatcatccctcgtctccctcttgcagttttctcttctgcaattctgttactctcacaataccttgtctttccctccactccctgtttcctctttcttttgcccccacacatggttctattttgcacaatcttcccttgatcttcagagttccctgttctctcccacgcttccccattaccttccacgtttctatcctgtctcatgcatcttgttgtctcagattctttcctgccacccatgtgcggtccacataactcctggtatgggttcccaatgcccagggagtatgggagtgaacagaccccagggcctccaatctgtcaggctggctaaaccttgaaggcggttccacagcttgaaggggatgcagggctaggtgtcacacattctctatagcttatgctccatacttcctggctggggttgccaatttagggctttgaaatgtcaaggaatccaaggacaatggcccagggcccaagagacgacatgcctccccgaatggcaggtctacacggagctaggaagctcccaagagcctgcttggacttgcaaggctcactgccgggagttgctaggaaggaggcctggcccttgggcactgtgaggtcaccagcagctgcacttcctgcaaggacactcaggtattcacacgccacggatgcagaatgagtgcacacctaccacagccttgttggatttgctaagaccagacgagaaacccgtggaaaggcagacatgctggaaagcactcggtgcagacagcaggccagctcagcccaggcggggctaggcttgggggttcaggggtcatctgcaatggtgctggcatctgagactcacagaatggttgtggcaagaagcggtctcctttgtggagcccgaggggacagcgaaggatgcaagaacttaggagaaacaaaaaaccacaggatgtggagcatgccccagagtgtcaggtcacgagggtgggtgctgagctgagacctcgatcacgagccactgtgtgaccctccaggccatggtctttctgcaagaactacagagactgcacacaagttcccacgagctcaggatgtgagggtaaacagtgtggggtgaccctgtgagaaatcaaagcctttgtgaagcgcctccagcaggaatgcggttttggaacgggaagtcatcttcccagctggtgcctggtctccagttgagctgtttcaaatcctaaaagatgacgctgtgaaagtgctgcactcaatatgccagcaaatttggaaaactcagcagtggccaaaggactggaaaaggtcagttttcattccaatcccaaagaatgctcaaactgcagctcaattgcactcatctcacatgctagtaaagtgatgcttaaaattctccaagccaggcttcaggaatacgtgaactgtgaacttccacatgttcaatctggttttagaaaaggcagaggaaccaaagatcaaattgccaacatctgttggatcatcaaaaaagcaagagagttccagaaaaaatctacttttgctttattgactatgccaaagcctttaagcgtgtgaatcataacaaactgtggaaaatttttaaacagatgggaataccagaccacctgacttgcctcctgagaaatctgtatgcaggtaaggaagcaacatttagaactggacatggaacaacagactggttccaaataggaaaaggagtacatcaaggctgtatattgtcgccctgcttatttaacttatatgcagagtacatcatgagaaacgctgggctggatgaagcacaagctagaatcaagattgccaggagaaatatcaataacctcagatacacagatgacaccacccttatggcagaaagtgaaaattaactaaagagcctgttaatgaaagtgaaagaggatagtgaaaaagtttgcttaactctcaacattcagaaaactaagatcatggcatctggtcccatcacttcatggcaaatagatggggaaacaatggaaactgagaaactttatttatttatttttttggctccaaaataactgcagattgggactgcagtccatggggtcgcagagtcggacacaactgagcaactgaactgaactgactgcctggcctccaggaggagcgcgttctctgctccagccaaacagctgctcccaccatcttgacaggcaacctcgactctcctgagatggggctgcagggtccatgcccgttgacatgagcctttccccccttctcccgtcttctaaattcctctgctctctgtgggctcgtcactcttggtgtgacctaccccgacctggagccacccggacgtatcttcatcctgccctgggaggaccctcccagcgcctctccccatgcagcccaccgtggggtttcatgtggtggtcgtggggatgctacaagcgctcttgttggcctggtgattaagaatttgctttatgatgcaaggaacaccagtctgacccctggtccgagaagatcccacatgccatggggcaataaagcccgtgtgccaccaactactcagcctatgagccacaaatactgaaacccatgctctgcaacaacagaagccactacaatgacaaacccacttacggcaactagagaaagcccacgtatagcaacaacaggcccagcacagctacaaagaaataatttaaaataatgcacttaaagcacaagcgactcaaaaaaaaaaaaaaaaaaatgttggacgtcatcgaaattaaaaaaataaaggaaaaaggaagtgctgtgaacgatggcatatccgtagagggcaggagactagttttagcgaggggtccaggacacctccttgagaatgaagtgacatttgagttgagacctgagtgcagtgtgggagtgagccacgagaacatcaggagagcttcataggcagaaaaggggatgtgcaaaggccatgtggcaggattgagcttgggtataaagaaacctgtgcctgaagtttgtgagcgagcagagggtcatgacgggaacaggctgcagccgtccatagcgtgtataggttgagagaatttgagatttggaggttttcagcaacgggagtcacctgcctcatgcacagttgaaaaggccacccttccctatgtcttccatggctcacacacacacacacacacacacacacacaccctagaacctggcacaacagcaggcaagcctgggtatttctgccctgagagaataatcttcacatctgttcccacaggttccagaggcaggtgtgtcattgggatggattaaacgggcctctaagggtacagttttggcgagtgatgcctggctttgattccactgattgctccccacacccttccccctcctccagccacatggcatcatctgtgaaaaggccactcttagttttaccaagagaaggcctccgggtacttcctagaattgtgcaagtggtggccaggtgaccatggagccccctgcccgagtgacccccccaccccccaggaccacagagcactgagatatcgggacccctgatttagcgcaagtcccagggccctgaagctgcatggtgtcaacaggcccggtttccatttgctctgcagcgtccacagggtttggctgagagtgtctcagggtaagggcagtggggtggatacagtcatgacatcagtcaagttgcccgagatgatccggtagggtttgctctgtcctttgggctgctttaatcccgcgtaggtggacgtattatgtatttttcttttttcgctttttaatacggccgtgcaagttgtgaaaggcattcccggacattgacactgacccgtgacagtctttcctcctccccaggttccggcggcagcaccattgaaatccacatcgcaagcgggtcccgcggcggaaaggcccccgactctcttccgaaagtccaaggaaccccgtagagaggacgagacagagggctctggaccctgtgtgtattttcagacgcgacggttctcatcccttatgacggtacccttgcccttcagtcagcacactgcggggtgtagcgtcccccccaactgaatcttcctgccatcacagttaacagagtgttccctggcagcctctgtgtgatgcagaggcccaccgtgagcctgtgcttggaaaggaaaggcagattcccttggagcccagcagcttgtccggagtctccgtggacgttcgtttctctgatctggctgtaatgtcaacgccagatccaggtccttggaagagttaataaataacaataattaaaaaaaagaagtagttgtgtccgagctcccagcctgacttggggtctgtctgaataacacctctaagcgaatgctcttgtgtaagataagtgatgatgcagggaccggggacgcacctgggagcaagttcctcacccaccactagggggcagaggagggcaggcttggaccaccaggttagccttcgctcggtcctcagctgtgttgagcggtagcgattcagtctacgggaagcttccattcagttccgttcagtggctcagccgggtcggattctttgcaaccccatggactgcagcacgccaggcctccctgtccatcaccagctcccggagcttgctcaaaggcatgtccatccagccggtgatgccatccaaccatctcatcctctcgtccccttctccacctgccctcaatctttgccagcatcagggtcttttccaaggagtcagttcttcgcatcccgtggccaaagtattggagcttcagtatcagtccttccagtgcacacccaggactgatctgctttatgatggactggttggatcttcttgcagtccaggggactctcaagagtcttctccaacaccacagttcaaaagcatcagttcttcagcactcagccttctttatggtccaactctcacatccacacatgactatgggaaaaaccatagctttgattatacaggcttaggacctgactctgcttagattattgcctgtatttccctctttccataattttacatatataataatataaagctgtcctttggtacccatgagaggtttgttctgggacccccacggataccaaaatccacggacgccctccacatctgcatggatgactgtttacagactttatgtattataaacatatatgtgtatatacatatttggagacaaaaatattgtacacactacacacaggagacacaagccacctaggtaagtctatggcacaagcacacaacgtttcctcaattctgctgtgtgatgctcattcacatcaagattaggactcttgtctcactctggaaaagctgtccaagctgcttctggatttgtgcttttggagtcctagggccacagacaggattcagggcatcccacaaccgtggccgtggtaccccagggccgcacgctcctaaagctgcacttattacctcacagccaccttgacccaaagtgtccaacagcagtaaaaaggacttttgagcctttctgggttcttgtcctctgtatcgatatcctgtctgccccagtcatcagagaagccccgagacctctcctctgagggctccccctgccgagcaagacacagccccaaccccattctagagcaaagatgctagccgctggcttccccaggctcccttgcagctagagtgtgtctggtgtggcggatgagacttgtgccccaagtcaacgttggaagctgatgcgcaaggaaataggctgtgcagaagcaattctggggaggggttggcagctgcatgtctgctttggaaggcagcctgggtgagccctgagaggtcagcccagtgtctggcatcgagctgtgggcagcaccccagtgctgtgttacctgcataggcctgattctgggcctggctctagccagttctattcgcactgtcccccacacctgggataccccagagctgctcaatatactcctcagccattctttctggcttacgttagccagagtccagtggctcagctggtaaagaatctgcctgcaatgcagaagacctgggttcgatccctgggtcgggaagatcctctggagaaggaaatggcaacccactccagtattcttgcctggagaactccagggacagaggagcctggcgggctacacacagtccatggggtagcagagtcagacacgacttaagcaactaagtttcacttgcagttcagttcagttgctcagtcatgtctctttgcaaccctatggactgcagcacaccaggcctccctgtccatcaccaactcccagggtccacccaaacccatgtacatgagtcggtgatgccatccaaccatctcttcctctgtcatccccttctcctcctgccctcaatctttcccatcaccagggtcttttccaatgagtcagctctttgcatgaggtggccaaagtattggagtttcagcttcaacatcagtcctttcaatgaatacccaggactgatctccttcaggatggactggttggatctccttgcagtccaagggactctcaagagtcttctccaacaccacagttcaaaagcatcaattcttcggtgctcagctttctttatagtccaactctcacatccatacatgaccactggaaaagccttgggaatatatagccttgcaaagtgtttaaatgagcctactcattgccttattggaaagattccccacctcctcattcacaatcatggttttggtggagtttctctcagcaactgggctcaagcataaatgtttccctttataggagctcccatgctccagagtattattaacttcaaggctcaggggaaggtaacacttttaacagttcctgcctgccccaatccctgtggagtgtccctcccctcaacttcccatgccgctctgcgggtggacctgtatgttaactttgcttaccttgcacatggatgaagacactttgcagaaacagggagccaggctgcggaaaaaacaacaaaaattccacccaattctgagtctttcgtaaaggtctgtatctgctgcccacgccttcccatgcgtgtgaactagtgatttgcaggctcccaacactgacagtgaccaacaccgcgcccacaggtgcacagaaccctcgccgtgagtatgaacacctcaaagaccttttaggaacatcacccagatgtgggctcctaaaaaaagaaaccaaacaagttctctgctccgtgagtggaaactcagattcccaaatcaaactggggaagggggagggggttctcaatggacaaatatttttagaatcactgcagcagaaggttccatagagatgaaagttaggtaaaagctcaggcaatctggtctcaacccagtttccgtcacagcacagagacctccttggaggtgtgcctgagctaccagcgtgttgcttcagctacggaagcccgagttccaacacactcgtttaacagtggttaagtcccgccatgcgacgtgcaccccagtgggttcgggacatggctcaccccagacccttcagcagcagggctcgggagtgctcagagattcaccccagattcgctgcgtgaaagcaccctcagtcagctaggcaaggcttacaaaaaaaaaaaaaaagaaacaaaaccaggcaggtctgcaccccgggcaacactggggcagaagcaacagcccaggtctggcgtgattcccatgcagggtcagcttgtggccggaggacatggggctctttggggtgactcgcatggccaaatgcagctcgtctggccacctctgtgtcagctccacactgcctatcacccgggggacatcgcttcaaagggaaccggtcaccgtctgcatgtgtctgcctgaaccgtgtgctgcgcccggtcaccagcatccctacaacttcaggttcttctattattcatcctgcacctcaaaactgccagcgacccaagcctgcctgcaggagaacatccgtctgctttttgacttttctaaaactgccagcctgaaacaaagtaatgagaatgaaactagttacacacacaaaaaaagcacactcgccttttacatcataacgcatctgcaaccgctggtctcttcatctggtttacaacgaactggttgtaaaagcagccctggcccccgcccatttctcccaggggaaaaaaacagacaagagcttggggacttgaggcagcggagttagcgtgggccccccagacgaacaagaaagtacacacagaaagttctctatcaaaatagagcaatttatttaagataacctaatatggtctgttgattctctcttggaacaaatgagccatctctctcacacacacacatggtgattcacacacacacacacacacacacacacacacacacggtgattctctttccgggagcgtgtgagggtgggggttgcagtggggcattggtggagagaacggaacggggtgaggggtgccgtgtggatggaacagaagctggaagatgcggacaggacatctggctgtacgtctgacacagacaataaaaaccgcagaagccagacattcacaaacctcagggagcggcgggaggaaaacactctgcattggatgggaaacacgatcagaaaaaaaaaaaaaaggctgaaaggttaaaacaaaacaaaacaaaaagtagacccttactcccgacaaagtgcacttcactcggctccgagatctctcttcagaaagacaaagtgaacatcgcggttctgtagccgtggagtgggctcggacgcgcttccccgggcgtgcgtgtgtgtgtgtgtgtgtgcgtgcgcgcgcgcgagcgagcagaacgagctccccggcaaggtggacaattccggggatggatctgtgcgttactctctgctttcagcaccaaggtcccatctgcacactgaggactacgaagcgttctacacgaagtctagaagccgctcgcactgacgaagcgatgcgcccactgagcgctggcagagggtgcagcgggtgtgactcagggcaggcatgatttcagaggcgccggctgcctcctcctcgtggtccccatcccggggcctcttcctgatggttcatgggcagttctgccttcctcagcagcttcagttcatcaccacgcccggtatccagtccgtgtccttggctttacagcccctggctccaacccggcaacttccagcctggtacaggtttaccaggaggcccccgtcaatacttgggagaccctgcgtaccaacctccccgtgacgatcctctcgtgggccttttatatcaaatcccaccttcaccaccctccagcagcatttggctcatcaaatgcaaaccttgtctggaccccgggcagctgatgctgtgtggacagtgacacgttttcattcggggaggggtccagatggtgttcaactagacctctgcaaactgcatctcaaagttaatcattttggggggggtgaggggggtcagtgattatccgtggacgcctgagggcgtctgctatcttcaaaggtgggagtggagacgccaatcaaacactgaccttctgaaagtttgagtgtttagatacagcagagcctgagaacacgcctgacctgtgaagagtgcctgttggtcttctgcagtcaagggggtttgaaaaactctcagatccatggagatagatagtgagctgagagagaggaatgcactctggctttgggggtgctcccaggacaatccaacttctgcacaaaggcctagccatgccttaccgttggcttcggggtccatggacagataccaccaccctccaaaaccatccaggctggttgctggaacacaaaagggctgccaacctacccaccgccagccccatcccggggggaagacggataagcccagaggccctgctgctaatttacaagctgggtcagcatggggtcaggacagcagtagaagtacaaaggttacctgatccaagggaaaggaaaactcccctagggacacacaaagccatgttttaatttttttttttttttaaaaacagtaaaaagattcattgtttaaaaaaaaagggcaatttctcctgacctgctgccagaggaagcaaactgtgaagatggcctgtgaactgagacaccctacctctgttaattcaccctcaggactgaaaacctctctccaatcacttctgtcgctgtgagacccagtgaggtcccggaggcccctccagttggtcagccctggagggacttggccgagctgctcctggtttggagggggtcccccccacccacgcccacgagtctcaaacaaggcggcattggtgtcgtggagcgggatggttccgacggccccccttcagcgatgctggatctgggaacgcttcttgggttcgagggcacccccttgtccgctgttctggtccaatgcactgggaagaaagaaacgtccaggtcttcttggagcagaaaaggggctggggtcttcctctccggccgcacagaccagccccaaagccgaccccagagctgcccacccggagtcccctcactgctttccaccaagttcaaagtgaagggacccctctacctgtctcttcctaaaggcttctttttttgttgttaaataggaatcacacaaagctctcgtgtctgaatggccattttacgattttaaaattgttttaacataaaaaatatcttttttctcttctccaaaaatactccgggctctttctcttataagtcactttttttttttcttcttccccctccttgtaaaaagtcccccttgccctgccttcttacataaagcacttattatgcgccgaaagtgcccttgagactggaagatgggaggcaactatgagggggggaggggkaggggggaggggggaggggaagatgcaggccagaaagcaccgcatctgagaggggctgtctctctggtgggtgaaacaacagcaaataagcaagacacccagtaacagtctccacctgggacccaaggcccgccccgcctccaccacccttccaccccaaccaagacgacaggattccagcccatagaaaaaccacggggacaaaggttctagaaaatagaaactgttgggattacaaaggtacccatttcatccatacaaactggtctttcgaacatccttgtgagagtttaactgtagtgtccaaatgtttaggggaaaaaaaaaaacaaaaaaaaacaacccattgacgggaggagtttttcctccccttttggtttatcacagcatttttttttctcttttcattttggcacagcctttcctgttttttcgttcgtctcaaccatcggagcctgttctgggtggcaattgatccatgcactgagtcaagctggtggcttctcgctcccctggggctggacgtttcaggtggaaaccattccttttccccgcctttgacagcacttgcgtgtgtgttttctgttttaaacttctcggtggaggttatgaaaaaaaagaaaaaaagaaaaaatagagactccagtggccagggatgacttggtctcaaaggacactacctaaggtcaggtttgcgctgtttcggagctgacggaattgtgtctgaatagtgctacaaccccacacatccactgctggcccgtcggaattctcttctcatgtctataatatcttttctgtgtgtgtttactttcgcagatctaacacgcacacctgaaaggagagagaaaagcgcctgttatcttgagaggcaacatcagatgcccttaagagcacaggatggcacatggcctgtggaccaaatccggcccgccgtccacttgtttataaataaagttttattcacacagtccagagggttcaatcagaccagctctagcggacaggtgagtgtctaaacatcattagcacccaaccctaggccacagccgtcaatgggtgtcacagaccagacccctacatggtggagcagatggcaactgaagcctgagcacacagctcgcgtaagggtcggggggcaggctgacagccagccagccaacgccctgccctacatctcagggcaattccaagtgcagaaagctagacacctcaggggaccagcagagggttactgggacccgggagacctgttgaatggaaggttgggccttcatcaccagagggtctgggtgggggcacaaaggttgaagccaagggtggaccaagttaactgctcagcctcatcacctgtcatgtggtggtcacttctccgctgttggggggaggtcctgagcaccctaagacttccagcagcagccccggtttccacaccaggtgccagcaaccccccacccacccccccaaaagcactaacaacccaaatgcctccagacacctccaatgtctccaagggtgcaaccgatgcagctgagaaccacgaattccgccaggaaaaaaaacacacatcattcaggaagcaagaagagacattcactcccagagcactgcccagcccagatgcttccttcaccaacacacgtctctgaggcctgtcacagccctgggagtactcctacgtgggtctcaggggtctggaccgttgttgaccccaccctccccgaaccctccctgctccccgcaccgccccctctcctgcctggggaagccccaggggtgctgcttacggagccgtctgacgcactggcgcccactttgtcgccccgggcattccagcacacctcgaagatgcccccggtgcctcggtagctgtgcacgagacttccgctctgcaaagcaagggcgccgtcactcgagaggcggaacgctggcgaggccacctgcccaccgaccccacctggggatgtcagcccagaagcttgcaaagccacagaagctaatagaactttcagttaaaggagctctggcctggcgggaacatgtgactcagatctgtagccgcagtgtggggggcctgcctaaaggacccttggctgcccaccaagagagacagcctctatggtttccgccaggctcacgcctcccgggggaaggagaaagtcagcaagattggatacgtgcttatatttcaaccaggcttccgtctcactgagtgttcaagggagcctcagaataactcaaagagttttatggtttagtgggggagaaaaaggatgcaaaatgtgggaagctgctgtatatatatacagcacagggacctcagctcggtgctctgccattacctggaggcttgaagacgggtggggagagggagattcaagagggaggggacatatgtatacatatggctgattgccttgctgcatagcagaaatcagctgtaaaaaaaagcaattatcctccgatttaaaaacaaacaaaaaaaagggtgagtaaaatgatttgctgagctgctgatggcaggcagaacccagctattgatggcaatttttttgaaagtcaagcatcctcctgtgatgcgagttcgatccctaggtcaggaaggcgcccctggagaagggaatggctaccctaactccaccattcttgcctggagaatcccgtgcacagaggagcctggcgggctacataatataagtccatgaggttgcaagagtcggacaggcctgagcgaccgagcacacacatgggcggctgtcagcctggcttcacattagggccactgtcgtgtctcatttgccaagaaggcacgattgcgtgagacggccgcagaggaggagcccgaggtctcagtggaccaccctcggccccaggctggctaacgtggcatccgttactacctcccttgagaaagcatgttcatcagtgtctgccaggcctcagggcacaccaacggggtgcccgcttcatccatcctgggaccacacgcaatgcctgctgctgtcaatggaaggcagtgggtgcaaggtttcctgccaagggctcggaaaaaaaataacccgagtccctggcaggggcgacccagcatccacaaagagcctggggcagatggtgctgggtgggcttccgaccgtccggggaactggggatgtgtctatgtccgtgtccgaggcaaggcattcctgagtgcaaactcaggttctggggtcaccaagaaagtgaaatcaaactgtcagtcgcctagttgtatccgactcttggcgaccccacggactgtagcccgccaggctcctctgtccctgggattctccaggctagaacactggagtgggctgccattcccttctccaggagaatcttcccaacccagggatcaaacccgggtctcccacaatgcaggtggattctttaccatctgagccaccagggaagcccacgggtcagtgagaacttgactgtattgggtgtggggctgggggggttggtggggacccgggcggtgggcatgcagggagtttacctgagtgttccagatgtgcacacacttgtcgaaggagccgctggccaagtacttcccgtcggggctgaaggccacgctgtacacgggttcctggtgcttggtcagcgtgtggaggcacacgccccgctccacgtcccagaggcggacggtagaatcgaacgaggcgctggaaagacagagatggtcaccgcggacgcctgtgggttcaaaaaaggccaccacagctgtcggcaggttcccgccggtctcctgaggacgtgactctcacagtgggagtgacttcccttacatctcaaagaccgtaagtgggtatttcttcctcttctagaatgtgactcaacacgcactgaacacaaagccacggtccagggaggggacatgtatgcctgtggccaattcatactcaggtacgccaaaagccatcacaacatcataaagtttttctcttccaattaagataaataaattttttaaaaagtcatgcaataaatctctttctctttttgggtgccattatcaagggaacaaattgacagccacactcatttatttctgcatttctgtgagacacaaatgctacgtatttgctaaattttctaaaacggtcccaccactgacaacacatgtatcgctgccaagtgaaaaacaaagccaaaaataaaaagccgagagccgtattcggggaaatggttaagtgagcgaacacaaattttaatcccacaggaattaaaatataatcgcatccaaatatacaatcacactcccaggagctgcaaattttagttatgtctaaatctgaaagaagagcgtttgagtgcatgcgtgccaagctgtcccattcatgcctgactctttgtgaccccatggactgcatccccgccaggctcctctgtccatgggattctccaggcaagaatactgcagtgggttgccgtgctctcctccagggaatcttcccgacccagggatcaaacccacgtctcttacatcttcagcattggcaggcggcttctttaccactagggccacctgggaagcccacccaccacagagctgtttccccaggcatttctttcctcttctgggacttacatccatccctttatgcttggcgcctagaaggctcacttacggccgtcatgggtaccatgtgattcaaaagcacctcacaagtcatcaaacaaaacaaaacaaaaaagataacgttctgttccagacctttaagtgcatttcaggccagtggcttgcaataggagcataacagctctcatgtttttctggatcagcttttagctgatacaggaattcctttctgtgttttctgagagacggccttgggtcaggcactttcagtaacagattcttcatcccactgctatgtgacaaagacaaggggagggggtctccccctcatttactgcagggctgggccaccagccattctccccactaacccattctctccaattctgcaggaaaaaacccactgccgcttcctcccaagtcctgtctttcccaggcgagaccacacaaccatgctttgtgggatcatggtctccagaaccctcggtgtgtcccatgttcactcctggaatctctttagcttgtgtgcctcaaggttcttattaaaatatcctactgggagtgcaacactgtatcctgagtgaggtctgagcaggactgggaacacagaagtctgcagaacgccgcctcagtgaggagcttttgtggacttctaaccgctctgctcccttctggtgtgtcactgactggctagtcctctgcatccgatgctggtgtcttagggggtttactgcacacaccaggggtccccatcctccgggacctgatgcctgatgatccgaggtgaagcagacgcgatcataacagaaataaacggcacaataaatgtagtgcactcgaatcatcctgtaaaccttggaaagtgattttccataaaactggtccctggtgccaaaaaagccggggacagccaggacgaaaggttaggctggatctggtattggccggggaacctttatcaccatcaacactctcaccacctgcagctccgagttgggccaagtaaaccaagcccgaagcgaagtgaaatgaactgaaagtcactcagtcgtgtccaagtctttgcgaccccttggactatacagtccatggaattccccaggccagaatactggagtgggtagcctttcccttttccaggggatcttcccaacccaggggtcgaaaccaggtctcccacattgcaggcggattctttaccacctgagccaccagggaagcccaagaatactggagtggatagcctctcccttctccagaggatcttcctgacccagggatcgaaccaggtctcctgcattgcaggtacattctttaccaactgagcttccagggaagcccaaccaagcctggggttcaagctaaactgcttcctttcttcagttgtagtaaaaaccttcaccaaagcaaagtgctgggagggtggatgtaagcaattcctccctccagcctcaaggttaaaagcatccttatctcaacttagctatcatgtgataacctctatctcctggccctttcactttgagttaagtttctaagtaactctgaatgtgaatcactcactcgtgtccaactctttgcgaccccacagactgtagcccgccaggctcatctgtccatgggattctccaggtagctttgtcccatctccaggggaacttcccaacctgagtctcctgcattacaggcagactctttagcatctgagggcatgaacacgtttgcaagacacactgacagcatctcctggtctcttggggacacgaatgacttttactgggcgcatggctggcgcagcctcagccaccaaggctgctttcgggaaccgagactggggtgcgggctgtctgtcctttacctcgccagcatgatgctggagttggggttgctggtggccggcccggtgggactccacttgatggtgtatatctctttgctgtgcgcctgaaggtcgtggacacacgtgtcctgcttcatactccagatctacaggacgcaacgggaacacggtgagcaaaggccgattcttccactgcggggagcttcggcaccgttcctgcctggagcactgcagaggtttctctgtccctgggaagctgacgtccagcggggtgggcggctgacaaggggcaggtgcttcacaggcagaggcgtccgctgaccaccagggggcgcccgtgagaaaggccagagagttccatagcaccctggtctgatctcggaagctaggcagggtcgggtcgggtcaggtctagttagtaattggctgggagaaaggcctcgggaacggaaggaggctccacgggacctcgactgataaacagggcttcactgccggggcaagcgccacgtcgtttgcaaagagaaaaaaacgacagaggcagcaagagtgctcacagcacaacggccagctgcaggaggagcgctgggaccgaagacacagcacgaggccccaggggtcaccaagggtcacacggcccagccttgcagccttaccttcaatgtcatgtcatcagagcaggaggctagcaacatgccagacggatcccatttgatagcgttgacctcattctgcaagagagcaaaaggtttaaaaaaaaaaaaaaagaaagaaatgaaggcctgggtgtagtaaacacaagagtccagcagagggcaggagcgctctgtcttctctgcaaaaagcctgggagtagtaaacacaagcgtccagcagggggcaggagcgctctgtcctcgctgaaaaaagcctgggagtagtaggcacaagcttccagcagggggcaggagcgctctatcctcactgcaaaaagccaggaacctgtaaaacaagttgaatgcagaatttcaaagaatagcaaggagagagaagaaaaccttcctcagcaatcagtgcaaagaaatagaggaaaacaatagaaagggaaagactagagatctcttcaagaaaattagagataccaagggaacatttcatgcaaagatgggtatgataaagggcagaaatggtatggtcctaacaggagcagaagatattaagaagaggtagtaagaatacacagaagaactatataagaaagatcttcatcacccagataatcatgatggtgtgatcaccaacctagagccagacattctggaatgctttaggaagcatcactatgaacaaagctagtggaggcgatggaattccagttgagctatttcagaatctaaaagatgatgctgtgaaagtgctgcactcaatatgccagcaaatctggaaatctcagcagtggccacacgactggaaaaggtcagttttcattccaatcctaaagaaaggtagtgccaaagaatgctcaaactactgcacatctgcactcatctcacacgctaataaagtaatgctcaaaactctccaagccaggcttcaacagtacgtgaaccgtgaacttccagatgttcaagctggatttagaaaaggcagaggaaccagagatcaaatggccaacatctgttggatcatcaaaaaatcaagagagttccagaaaaatatctacttcttactttattgactatgccaaagcttttgactgtgtggaccacaacaaactctggaaaattcttagagatgggaataccagaccacctgaactacctctggagaaatctgtatgcaggtcaggaagcaacagttagaactggacatggaataacagatttgtaataaacagggaaagagtacgtcaaggctgatattgtcaccctgcttatttaacttatatgcagagaacatcatgaggaactccgggctggaagaagcacaagctggaatcaagatgggcaggagaaatatcagtaacctcagatatgcagatgacaccatccttatggcacaaagtgaagaagaactaaagagccttttgatgaaaatgaaagtggagagtgaaaaagttggcttcaacattcagaaaactaagatcatggcattcggtcccatcacttcatggcaaattagatggggagggaaacagtggcagactctattttttggggctccaaaatcactgcagatggtgactgcagccatgaaattaaaagacgcttactccttggaaggaaagttatgaccaacctagacagcatattaaaaagtgcagacattactttgccaacaaaggtctgtctagtcaaagctatggttcttccagtagtcatgtatggatgtgagagctggactataaagaaagctgagcaccaaagaactgatgcttttgaactgtggtgttggagaagactcttgagagttccttggactgcaagaagatccaaccagtacaccctaagggagatcagtcctgaatattcattggaacgactgatgctgaagctgaaactccaaaactttggccacctgatgcaaagaactgactcactggagaagaccctgatgctgggaaagattgaaggcgggaaaaggggacgacagaggatgagatggttagatggcatcactgggtcaatggacatgagtttgagcagctccaggagttggtgatgggcagggaggcctggcgtgctgcagtccatggggttgcaaagagttggacacaactgaccaacggaactgaactgaactgaactaacacaagcttccagcagggggaaggagccctctgtcgtctctgcaagaagcctgcttgcattcaccctggataaccaagcagcaccccaggtttcaggcatgtttctctttctccttcctccttgatccaaagtgcacagtcatacgccctacacagccagccaccctcagctatatttctccaaggggggaaaaaaaaatcacatctaagaaggaagccaaatgaatttatctggaaaccaatgtaaaaccagtttgttatttcaaagctgactttaaaaaagtaattttcgtacttttttttcccctttatgtcttcagacatttttataatacctactttagtgtctttcctgcaaaatccaacacctggctcatgccagataattaaaaaagaaaaacaaaacaaaacaaaaacaccagtgccaacccaggtggatgactgaccaccggaggcagcttcaacagtcccaaggaggctagaggagaatgagttggcagtcacagtaagtaggcttttaatcctaggcagagaccagccggtttcccagctccccacacacctttcgtgaacccgaggccccacactcaggactcacactcaccgtgtgtccctggaatgttttgacggggcggtcacagccgagtctgcagacgtgaatacacatgtccgtgctgcaggaggcgaaggtagtgttgttctgccagtccacgtcgagagcaggggctgcggagaagcaaagggcggaggggtgggggagagcggacaacacgggcattgagcaggtgtgcacagagctcagctgactcagaggcggcagcagcagagctgagaaacctgacaatgtgattaaagcaagactcgcggtgcccacacagcctcggtgcatcacggaccctgaggtctgcggtttacatttgcaaggatgaccagacgcctccagaaagagagccgacccccaccaaagcgtcacccaaagtgatgctttcggtgccctgagcacacagcaactcgctcccctggtgcaggtgagatggtaggggagaagcaccttgctgctcccgggaacgtcaccgggctgggtgatagattttctttcggtggggaagggagagagcggaggaaaaagacttcacagcctatgttcaacatgtgtgtgtgtctctcgctcagttatacctgactctttgcgaccccgtggaccacagcctgccaggcttctctgtccatgggattctccaggcacaaatcctggagtgggttgctattcccttctccagggaatcttccctgatccagggatggaacccaggtcttccgcactgcaggcagattctttaccatctgagtcaccagcgaagccccatgttcaaggtatcagggttgaaagaaatgccccatgacccgttactgcattctacaacttgaagatgctgagttgacgagaacatctgaggggagacatgtggccttaggatggacaccgtctctacgtcctgggcctttctcagaggccgctggcttgaagacctccgtgagggtttgggggacatgacggcttgcttcccttctgccatcgtccccaggagatgactcagcaccctcattctctccggacacgtccctgtttctacagaaaaggagggttaagtcctgcaagcctctgcacagaactttgccaactgatcaatacctaaccttgtctcacgtgtttcttacacgtaagtcagccctgaagtgggacgagaatccagctcacccaggacatgataaatccgacaacccccttctaaccttagcttcaacatatatttattaattttttaacatgtatattttggccagctgatgcaaagagccgactcattggaaaagaccctgatgttgggaaagattgaaggcaggaggagaaggggacgacagaggatgacatggctggatggcatcaccgactcaatgcacatgagtttgagcaaactgtgggagtcggtgatggacagggaggcctggcgtgctgcagtccatggggtcacaaagagtcagacacgactgagccactgagcaatttctggctgtgctgggtcttggctgctatgcgggtgactgtctagctgaggtgtgcgggcttctcactgagccagcttctcttatcggggagcacaaagtcaggagtcgtagggcacgtgtttacttgctccgtggcacacgctatcttcctgggtcggggatcgaacctgagtcccctgccttgcaaaggtagagtgttaaccactggaccaccagggaagcccctgttcttaaccttcaaagcagaggtgcaccccgtccaccctcctgccttccagaagtgcaggacagatcccatcagcttctaaatcttagcaccacccctcacagagcctgcagggggagggtttcacggagaggcaacactggctttctcaagaacccaaaaagggggttcagcacacgtccacggctcctgctgaatttgccacaggcgcccagtggaatgaaggagccggatccacttggtcagcaggacgatcctgggagtgtggaagcagagcccgcagctgctcttgtttataacaccgcatgcatgagccaagtcacagatttcccgggccggcctctgccttcaggcaagcaattaatacgcctggaagggtgacagtctggcacgcggttccggggatggttagcggcacctggccttaaaagcgaatgtgtgctgagccccggccatctaaacagaggacgctgtgtactcagctgaccggggtcgtcggtctgtgtgcactgtgtggctgggaccccgccctcccccgaccagtcacgagggctcagcggttctagggggtgtgggtgacctgctcacacttggccaatgacgtttgagcatcagcaccgcgctaggcacagtcgtgcttcacgttgcttttttccttttaacttttacttgagaaaagggtatagaattgatgctttggaactgcggtgcgagagtcctctggacagccaggaggtccaaccagtccatcctaaaggagatcaacccggaatatttactggaagcaaggatgctgaagctgcaatatttctggtcacctgatgggaaggactgactcattggaaaagactctgatgctgggaaagattgaaggcagggggagaaggggacgacagatggttggatggcatcaccgactcgatggacatgggtttgagcaaactccaggagatggtgaaggacagggaagcctggtgtcctgcagtccatgaggcggcaaagagttgaacgcaagtgagcaactgaacagtaacgacaaacagaatcccaggaaggtgcagagatgttacaggaaggctctaccttgtatccgtcacccactggttgcacgtcgcagccctgcagcactggctggacttggaaggtgagtgggtccagacccagctgtgtatgcagtgtcacagggtttgatctcaagggccgatccatgtaaccagcacggtggtcaagagccgcaagcgtgttccacatcaccaggacctcccttgggccatgttagttggcccaccatgctctccctggactgagatgtaaatgctatctcacccaggatgtgttccttggagtctgactttatttttttttcaaactgcagaagccctctgagatctatccaagcctttgggtattacccacctcatgagtcactcagttgtatctgactctttgcgaccccatggactgtaatagcccgccaggctccccgtccatgggattctccaggcagaacactggagtgggttgccatttccttctccaggggaatcttcccgacccagagatcaaacctgggtctcttaaattgcgcgcagattctttaccatctgagccactagttaggtcctgtctgagccacgatttgaccctcatggctcgggcagtattatcaacagtctatcaataaaaatgagtgggttgccatgccctcctccaggggatcttcccgacccagggattgaacctgggtctcatgtatcagaaacggattattttctgtctgagccacgagggaacgtcctgcacttgtttttaacctgttctttctcagtgctgagcggtggaaccctgttaagctctgaggcctaaggtttcttgctgaccgagggcacccccaccaagccttcatggggaacagctctggagaggcaagtcaactgctgattatcaacgcatcacacactgagggatggttgtccctacagcttaaagaaaagcacgctcccttgtcttccatcttggacagtgtctaccacaccacttcacggtatagataaagagcaaatagcacccagagccaacccagcaatgggatacggggcgacccgtgggcagcaacaccccaggtgagaagctccctgacggcagaagagacacgtggtccgtgggatagaaagcctgggaccttgggaatgctctggggaaaatttacatcctgctgaaagactcaatacaagagtcctcagggtgaaaactggtacaggaaacttatgggcatattcccctctgaaaaaatcagaagccagagctgtctgtgttacagacatccccccatcacgtgagcacactggtgctcagcacgaagaatcagaactctggcagggttctgccccgtcgctgagtcccttccctacaacagagcttgagatgctcttagcctctgtaatgaggctgcgatccccgaagggggctcacgccaaacccgacagaaatccagcaggctcacctgccacggtctgctctgtccatggcacagacggccggctcggaggaggtgacacgcatggatcaaatcatggagaccgggttgtcaggggctgggagagggcactagagagcactttcttaacaagtaaggggtatccttctggagtgacaggaatgttctggagctagacagcggtgaatgctctgcacattctgcccactggaaacacacacatggacacagacacacaaacctacacacagaggcacgcacagagacacacggaccaccccccccacaagagacacagaccccacaaacagagacacacacctacaaacacacacgcacccacacagacacgccccttcagagagatacccacacccacagaccccccaaagatacatcccttcccacacacagaaacacatccacacacaaaccccttccacaaagagagacacacacccacacccacacacacacacacagaccccttccacagagaaagacacacccacacacagacacacacccacacacagaccgcttccacaaagagacacacacccaaacacagacacacacccacacacagaccccttccacaaagagacacacacccacacagagacgcctccttcacacacaaagactcccctacagagagacacatacacagaccccccctgcaacacatacacattgccacacagaaagacatacacagaaacacacaaacagacccacatacacacactcacaggttgggcgcccaagctccctgctcttggagccctgcccacttcctggtggggaaacacaattctacaaaaggctcagcgagaacacaattctacaaaaggctcagcgactcccaccttcatcactgctgttaagagtaaaacagtatgaaacacccccagccagcacccccagagacgtgtgcagcatggagtccccgagcagagccacggaagacatgggctgcctgcctctccagcagggcgaggtatgcacggggcccctcccggggcctgctggtcgaaccaggagggaacgaaggactgcgtgcctttgtcacagtcatgttgagggccctgcggggaacctctcctcccaggctgcttctgcaggaccacacccctcccctcctgcttggagcccttcccaggcagcgaggtgggtcgcgctagcttggggagtcctgggggccagccctgtggttctgagcatcaccaggaacctggatctgcccagtaggagagaacctcggatggcgaccaacacatcacctcagggccactcaatgcatcacccaacgtccaccttgcatgctcagcactgctcatgaggcatgccttcttccgagcattcaccgttacgttaagagaccgttctcaggactccgaattcttatttttgaattaaaaacaaggaaagttaaaaaaaaaaaaaagtattaaaaaagtactcaccggaatgaaatggaaactgttgtttggcttctcctgtgtgagcatcccagattattgttgtctgtgctccgcaaaaaaaaaaaaaagaaaaagaaaaagaaaaaaatttagttactggttcctcccggaaagttatctccagaaaaatttgtcacacatgagaacacagcgagcccttctttttttctaaactagcactgctcaacttaaaattttctagtaaccacagtaaaaattaaaaaaaaaaaaaacaaaaaaccaaaacagtaaaacttattttcacaatatacagtttatttcactccagcacagtgaaaatgtcacttcaacgtcaatcaatatattcgcagcatgtggcctgtgtcttttatgcccactctttggaatccagggtgtgtcttacacacttaaatcacccagacatcacacttatgaactcagccacgcctgtctgattatcatactcctccggacgacagcagccgtaactgataggcaggaagctaccaattttggtgggagtcccatctttatcctcaaacttgaatatctatcattaaaaaaaaaacttttctttttaaatgaagagatttttggttgcacaggacgtgagatcccagccatgcatcgaacatgtgtccctctgcactggaaagtgtagagtcttaaccactggacccagcagggaagtcccaaacttgaaaaattattaaaaaacaaacaaacaaacaaagcatatctaaaactccagtttggaccaattttccaactgaaacactgcagaaatggttatttctgaaaatccccctgaccgtcactctgtgaagtggggacactacacaggctagaggaactgggttccactggggtctggccctgatgtattgctgtacatattaagaagaagaaaaaaaaaaaaagatctccaccccagaagacagaggaagtcacgaatgcttctgaacacattatagtgttggggagacacttggggaagaagccggaggcagttcaatgcaaaacatttggaattaattctcaactggaattttcagctaaattggaaacttgacgcctccctgtcattgacattttaacaaagatcttcctttaggaaaaactggctctactttgtatcaacagcgctaatgttcctggcgagatactgacagcactgagtgtgtttgaaaaacttcaggccagctccagcgctaagtcgcttcagtcgtgtctgactctctgtgaccccagggactgtaagtccatcaggctcctctgtccgtgggattctccaggcaagaacactggagtgggttgccatgccttcctccaggggattctccccatccagggatggaacctgcgtctcttatttataagtctcgagcattggcaggcgggttctttaccaccagctccacctgggaagctctggaccaattaacatttttatcccccgctccaccgtgtggtttatagcattttagttccctgaccgggtactgaacctgggccccgagcagggaaagcgccaagtcctaaccactggactgccagggaagtcccagagattttttttttgatggagattggtcctgagtgttcattggaaggactgatgttgaggctgagactccaatcctttggccccccgatgcgaagagctgactcatttgaaaagaccctgatgctgggaaagattgaaggcaggaggagaaggggacgaccaaggatgaggtggttggatggcatcaccaactcgatggacatgagtctgaataaactccgggagttggtgatggacagggaggcctggcgtgctgtagtccatgaggtcgcaaagagtcggacactactgagcaactgaactgaactcttttttcttaactgagccagtcacaagtctggcatggcagagaatatataaacactttcacaattctgacacaacttcacataccacatcagttgttgtaacaacttactatcagttcagttcagtcactcagtcgtgcctgactcatttgctatcccaaggacctcacaacttactatgtgttaccttaaacacacactggacaaaagtaaacatgacttatccaaaagcttttatggatcagaaatacaggttagccactctgggttaagcacgcaccgagggccaaagccagtatccctaacttaaatttcatccctgagaagatatcattttcaagaacactgttattattcctgccacagttgattcatcatggtagacagtctttcactcagaggttctagaattccagaagatcaaaaccacagaagctcacaaaacctccaaacaaggcccctttgatttcttaagggggaggtagaatttccagaattcaaagaaaccgtgatgtttcccgagagattaaacatagctgaaaaaagcaaggtatctcacgcaaacactcactttgtctactccagcactcaaaatgtaattgcccttcttgttccacttcaaggcgaagatggggcctttatgttggcctaaggtgctggccaggttacctggtaggttaaaaacaaagagagaaatcagggtgacttctgagacatgctcggtctgtaccccagatgatggcaactgaaagtaccccctcttccaaccctgacgcgagggaggcggggccaggaggacttaccatcctctgtccatattcttgcaaaaccatcataggaacccgtagccagcagtgtcccgtcactctgtgggccagaaacacacaccaacacaaactatttattacagcctgcaggcagggcctccattccccgagcaggggtcggtcaaactcacggcccctgcaacagagctgcagactcctgacccctgggccaccaggggaagtcccaggcttccttcccccaaaatcatcacaaggccagggggcgagggaaacacaccgcaccccgtcaccaatgacgacaccaataaaagacaccgtacgaaaagttactgagtgactttgggagaccttgacgagctttgaaatagctgcagatactctgacaagagaagagtttgtgatgaaatgacaggacatccaggaacgtgataaaccgttggtgggaatgcaaactagcacagccactacggagaacagtgtggagattccttaaaaaactggaactagaactgccatacgacccagcaatcccaccgctgggcatacacaccgaggaaaccagaactgaaagagacatgtgtaccccaatgttcatcgcagcactgtttataataaatagccaggacatggaagcaacctagatgtccatcagcagatgaatggataagaaagagtggaacatatacacactggaatattactcagccattaaaaagaatgcatttgaatcggttctaatgaggtggatgaaactggggcctattatacagagtgaagcaagtcagaaagaaaaacaccaatacagtatactaatgcatatatgtggaatttagaaagatggtaatgataaccatgtatgcgagatagcaaaagagacacagatgtacagaatagtcttctggactctgtgggagaaggcgagggtgggatgatctgagagaatagcattgaaacatgtatattatcatatgtgaaacagatcgccagtccaggttcaatgcatgaggcagggtgctcggggctggtgcactgggatgacccagagggatgggatggggagggaggtgggttcaggatggggaacatgtgtatacctgtggcagattcatgtcaagtatggcaaaaaccactataatattgtaaagtaattagtccccaattaaaataaattaataaaataatatcaaaaacagaaaaaaataaaaaataaactgtacagtccaaaaagaaaaaaagaacgtgataaagagaaagtgaaaagatgctggagagctgtgtgtgtgtccgggcacagatgctgagggtcgggtgggaagttcgcatctccctgggcacacctcagggtacccccacataacaggcttcctgataaagccgccagactcttcctgccgccgacgtgcagcttactgaacaccatcaggctggcggtcggcccggacccccgggtgggctgacccacaatcccgtccccttcctggggtccgccccagctccgcccaacaccacaagctgtcaccgaggtcgtggggactgcacaggtgtccccgacggcgtccgggagggaggcagcttacgttccagtccagcgaggtgacgtctttgttactggggacgtcgtggcccccctcccggatacagtgcctcagcaccagctgcgtggagcccccgttgctgttttcattcaggttccatatcctggcagtcgagtctccggacctgcgcaaggaacacagctggccctcaagcttccgggcccagagcaagcaacacccactgttgcttgtcgcctggctgggtctctatctttttaatgtcgctccaaaaaaaaaatcaccgtggttttctctctccactcttaggtgccctcatcattttaaatgtctgtgcatagctttccacacagctgagaaaacggctgaatacggacttttgcacaggcttagacaccccaagggttcccccccgctcaaagccctgctctgtacgctctgagaagatttcacacatgacgtagaaagggagtgtctatcccaggaggaaggactcggacacctggaccccgggcaccccccaatctgatcctggaggactcggacccctggaacgtgggcaccctccatgtgaccctggaggactcggacacttggaccctgggcatcccccaacctgaccttggaggacttggacacccacaccccaggcaccccccaacctgaccctggaggactcggatacctggaccctgggcaacccccaacctgaccgtggatgacttggacacttggaccccaggaagcccccaacctgatcctggaggactcggatatctggaccccgggcacccccggcctgaccccttaccccgaagccaagaggtcactgacagggttccaggcacaaatgaacacctcggactcgtggccccgaaggacggtggctttgctgggcgggatctccacgtctccatctatttccattggtttcgagtgattatctgtcgcggggaacaggacaggacacgtgtctcactgacatggaagaaacccatccatcctcagagcatttaagcttgacagataacttcctccgctgcactgccaggtcacttgtgacaattggaaccaggtgatggcttctgtgaagtcttcacagtctccaaatttgataggggaattaaaaactggtgtttattctttgtaactagggccaggctcataggtaataacaatgggaatgagactgggcacaaacagacagctgtctcttgagcagaatttgaagctttcttctctgtcttagtgaactccgggagttggtgatggacagggaggcctggcgtgctgcgattcacggggtcgcaaagagtcggacacgactgagtgactgatctgatctgatctctgtcttttatgtctacagtaaaaagacgcatattttgttacattttcctaagaagtcaatgaggagagggggacgcagaggatgagacggttggatggcatcactgactcaatggacatgagcctgagccaactccgggagatggtgatggacagggaagaccagcgtgctacagcccatggggtctcaaagagtcagacaccaccgaatgaacaagagcaacacaagtgaaattaggagtttctgaatcccaaaggccgtctcactctttgttatatgcctagaacttaagttgtgctgttgctgtttaagtcgttgtgtcacgtccgtgtctttgtgacctggcttctctgtgcacagggattctccaggcaaaaatactgaagtgggttgctgtttcctcctccaggggatcttcccgacccagggatcgaacccgcatctcttagtatctcctgcattggcagatgagatctttactggtgaacaacttagcaactgaacaacaagtcagttgcaaatactgctgctagaatgcaagttacaattcaaatgacagtgagaataattaggatataaagtttcctcagcaaaagttccttctgttgacgggacaagtgattttataagacatcttcctgaggcgtcacctgcttacctacgaagctcccacacggacacgcaatccttcttccagaaggctcaccactcacaaccccttggcctgctaacaacctcatggtgttgggactccaatgctattttcttactggagctaagtgtgttccaggcttttgaagccatgcagacacagcccagatgacccaatggggactccaggttgtgaaaggatggtggcaacaagtcatggtagatgccgcacagaagagactcatcctggatcatgctttagcgttaagtcgctcagtcatgctgactccttgcgaccccatggactgtagcccgccaggctcctctgtccatgggatttccctaggcaggaatactggagtgggctgccatttcctcctccaggggatcttcctgacccagggatggaacctgggtctcctgccctgtgggcagattctttaccatgtgagcccccagcaaagtcctgaaaagtggatccactttttccccttctatgtggcacaagaaagaaggttcttgaaggtcaaaatgaagtctaacttcagacgctgtaagtctcgctgaagccgccttttaataagagcaggtgggacagagtagaagttggaaaacagtggacattgcatgcatgtgtatgtgtgtgttgcggggggaggctggtggccagccctcaggtggaaagtaaccccattgcagaggccccctcaaacgaaggaggagctgggagcttccagcgcagcttgaggtatgcacctgtctccacgtggctacgctcccttggcgttttagattgaaagacatggagtttaaaaagtttgcaagatttctgtaacagcagtaggtacatagatttctcaagcctcaccaattacgcttcctttctggggtggaaggtgaagaaaacggggacgttcaccttctgggctgtagctgaagcaaacgaggatgttcaccttcttcctgttttacttttagcaccttgacggagagaccccatgtgactgggcacacccacgaatgacgtgcgggcacaagccacgttatcttttcttctcctttcctgggaccctgtccttgcgaaccttgtgaacaatgattatggggcctgctgatgggaggtcatttgattcgacactgcacagacagtcttgaagccatcctgcatggcagcctggcccttctctctgcccagtggtagagccatgtcccaactccagtggggagaggcctcggcccggagatgacaatcttccccctcgggtgtgcacagcacttcagagggaaggggcrttcaaaatagggaagaagacacactcactggcagagacttacatgcatgtacacacacactctgaacgtagaacttccttgaagctgtctctgaatttgtaagcaggaggttctctggccttccctggcggctcggatggtaaagaatccacctaccaatgcaggagacgcaggtccgtccgatccctgggttgggaagatcccctggagaaggaagtggctacccacactccagtgctcttgcctgaggcattcaatggacagaggagcctggcaggctacagttcatgaggttgcaaagagctctctgcctcatttcgcacacacttttgggcattagttaacaagaaccctgctgcaaccaaccacataagtcggcattcgctatgagggcctggtaacctctcagtgaccatgggctctgagtgtcatgtcagaatctgtctgcagggcaggacagtgcagggcgcctccagagatgagacccttctgagccgggcggtgttcacacggcaacacgactacctttacacccagtgagctgtcaaagcagcagaactcctacagcagaggatgtgcaggtattagagagtcacgtcatcggtaccacgtatacaattagacagccagcgggaatgtgctggggcttcccacgtggcgctcatggtaaagaacccgcctgcccacgcaagagacgggggctcgatccctggcttgggaagagcccctgggcggaggaaacggcgacccgctccactactgttgcctggagaagcccgtggacagaggagcctggcaggcgacagcccacaggagtcgcaaaagaggcggaagtgacggaagcgactttgtgcacacacacgcatgggaatatgctgttgtgatgcagggagctctgtaaacaacctagggaggagatgggctgggacggagcttcaagagggaggggacatatttatcacgtgtatacctatggctgcttcgtgttgatggacggcaggggacctatgtgtacctatgcagacgctggtgttcatggatggtggaaaccaacacgatactgcacgttaattattctccaattaaacaaaccaaccacatctaaggacttcgatgggaaaattggagcaagaaggcctcctgggtgcagagtaagcaaacgctagccaaggcctgcagggcaccgtccagcggacccagccagagtcagaggacactagaggagcccgctggacccaggcatcccacactcactgatggcgtgtgctccgttctcctccccgttgactgtggcctcgccgttctttggtgggttctgctgggaagcggccgcgggcgctgccgtcgtggccgctgctgctgcggctgccgccgccgctgcagctgccgcggccgcagccgcctgctgctgggccagcttctcccggaaggcctgctgccgcgtctgcaccacgtccggcatcaccgcgtcgatcagggacagggactcgatggggcggccgtcgaacaccgtgccgtcctgtggacagagccccggggaggggggtgggtcagctccggctgggcgtctcctgggttggaaacgcaggtgcctggggccccggggaggcgtggggggctgggaccgggaaggggtggcccatagacacccagcactcggtcaataacaacctgaacccagacgtgagggggtcaccccaagcctcctatttcccatcacaaccggagggctgcagtggtctcaatggaagggggcgatcgtgctcctcagggcaggggggcatctgggaaaatctggaggcgtgtctggtggtcatgactgcgggggggtgcagctggcatgtagtggggagggaccagagatgccacccaacatcctgaagtgcacaggatgccccctccgctctgagaagtctctggctcaagctggaaagtcctgcgcccccccacccccaacaatcaggctctgactcatagcgtatccttccatctgatattatcaaacgcatgttctgccatgtgaagtgacttctggggactcaggatgcagacaaatgcagccacgaaaytgctaacaacggcaccgctgagttaccacgcctatttcctcagcttcctctcagtttcctagtttccatcggggatgaggaaaaaaaaaaaaaaatctcctacccttttagaaaacacaggagggagtaagagagtcctttcctgagaaggtgctctgattattagagtcacaagaaactccaggcctctcgctgtgcctgctagatgttcagacaggttttagaccgtttttccctcataaatcaggaagaactgaaggacgtacgtatcacctacaaagtgctcgaatgaaacagcaccccagccgactaccggatactacacgatatttgaaaattagsgaacaacagaaaacaatgtagaacgtggcaaaaagaagccaccctcatcccctgattatgcccatggagtggccagtgttgaccggctggccagacggcttctacatcaccttccttctttgcatattcatgccccagacggatgacgattttgcaaaaggtagcaactgcgcagagtaataaaacgcttccttgcctaaactctaaacgcacaccatctcactcctaacaaccggaaagctaagtgatcaatgagacgaccttgcagtctcctctaggcgatgttggactttgaattgacaagggagagaaggtatggaaatgaaagacgtggaaactgggtgtgtgtgtgtgaaagtcactcagtcatgtctgagttttgcaaccccttctccaggccagaatgctggagtgggtagctgttgccctctcgaggggatctgcccaagcccacgatcaaacccaggtcccctgcattgcaggtggatgctttactatctgagccaccagggaagcccaagaatactggagtgggtcacctatcccttctccaggggatcttcctgacccaggaatcaaacctgggtctcctgcattgcaggcggattctttaccagctgaaaattctttgcctttacaagggaagcccatatgtgtgtgtgtgtgtgcccatgtatatgtgtgtgtgtgtgtggatgtggatatgtgtctgtatgtgtgcctgtgtgggtatgtgtccgtatacatatctgttacacgtgtgtgcgtttgtgtgtatctgtgtgtgcgcagggaaatccaatcacggcgtcgcataatctgagagctggggtcctgtactcctatccccacatgtgttttgtgggaaaaaaaaaaagacttagatggggtcacgttgccagcaagaagatcccgggcaccctgatgtacgaaaaggaaagaggagattgcctgctgttgggcaggaacgcctacagtatccgcagacagccaggtgttctctgagatgcggggttttggaaaacgacagaggaacgcatgtgagagaatgtgagatacactacttgtgtgattgtaaaagcttaagggaaagtggaggtaaatgctacgaagaaaagacaggcctgcaaggatgagaaaggggaagagcaggcggaggaaagcgtggaagggtgcagggagatgggctcgagggtggtgtgatcgtgggagcagacttctggtagtgaaggggagggggcgcatggccactgaacggggtcagctgtgctggatgcgttggaacagaaaacggggacttggggcacagcggtgtgctggatggaggtggggtgtgggtaaatggaggcaggacgaaagagaaagggtggtaagtgcagttacaatcagagggagtcagactgagactccgggaagacccggacgtcacaggaggccccgggatccttagaggacagtccccagacagaagcaaagcaggtgggaaggggagctgtcttgacccctcaccttttatctttaaaaggcttcagttcagttcagttgctcagttgtgtctagctctttgcgaccccctagaatacagcacgccaggcttccctgtccatcaccaactcccggagttcactcaaactcatgtccattgagtcagtgatgccatctagccatctcatcctctgtcgtccccttctcctcctgccttccgtctttcccagcatcagtgtctttataaatgagtaagctcttcacatcaggtggcgaaagtactggagatccaacttcagcatcagtcattccaataaacacccaggactgatatcctttaggatggactggttggatttccttgcagtccaagagactctcaagaatcttctccaacaccacagctcaaaagcatcaattctttggcgctcaactttccttatggtcgaactgtcacatccatacatgactactggaaaatccatagctttgactagatgaacctttgttgacaaacctttgtctctgctttttaatacgctttctgggttggtcatagcttttcttccaaggagcaacaagcaccttttaatttcatgactgcagtcaccatctgcagtgattttggagcccccccaaaataaagtttctcactgtttccattgtttccccatctatttgccatgaggtgatgggaccagatgtcatgatcttagttttcggaatgctgacttttaaaaaatgttacaataacccatttataaactgctataaaattggccctgatatgcacatgccctgatctataaagagggagggtatttaagcaaatcaggagcaacttaaatgtacacgtctggtcccaacctcccggaagcagctgtgcagtgagagctctaacaaagccatcgcttgaaataaccctcttgaactcaacagtactcatctctccgcttcgatcttctgggagtgccctcgctcaggttcacataaaacacagactctagtgaagtgactcacagggtccctcttccaccgaactgctgagagacatccaggaggaagggtcatttccgtgcccgaggctggcccatgcccatcgtgaggcatggctgagccttgaacgaggcgggtctgacctcccagggtccactacacgcagatggacgtgtttcagcacgtttcccactgtttgccctccatggcgggcggaatccgcaggtacagaatggctgactgtgggacctgagcaccccctgtggattccgagagacgaccgtataaatcagcatccttttcaatgcatcatgatgaccgctttacggtgaacggtgtcagattcttgatctccaaagaagatttagcttcgggaccagggaccacgcttgatcactcaagagcttttgtgtagcagaagttttattaaagtataaaatcggacagagaaagcttctgacacagatatcagaagggggacgaagagtgtgcccctcactagcattgagttcagttgctccattgtgtcagactctttgcaaccccatggactgcagcacaccaggtctccctgtccatcagcaactccaggagtttactcaaactcgtgtccattgagtcggtgataccatgcaaccatctcatcctctgtcgtcctcttctcctcctgtcctcaatctttcccagcatcagagtcttttccaatgagtcagctctttgcatcaggtggccacaatattggagttttagattcaacatcagtccgtccaatgaacacccaggactgatctcctttaggatggactggttggatctccttgtagtccaagggactctcaagagtcttctccaactccacagttcaaaactatccattcttcagcactcagctttctttataagtccaactctcacatccatacatgaccactgggaaaaccatagccttaactagatggagtgttaccagtgtaagcaagggagctatatcctttctcattaattattacaataaatcaaaagaatgtctcaagtttgtgaaaactttaccagaccaactcacataatttacattgtaagataacagcattagccagaaggttttcaggaaggagaaactgtcctcaagcaggagatacactgttgttatataatccttaggacagagtttaaagtgagctgtttatgtactcatcagttcaggcttaaacaaacaaacaaacaaacaaaaaaccattttatgtgactaagacttaaggaatgtcaagggggaaaaaaaaaaaaaaaagatgtttgtcctttcctcttccttgagaatttcaggcccctatcttcaccttgagaaccccagacccctttctccttctcctcctccttggggactccggacttcttatcaacctgcctaggcattgcctctctcagtaataccactgtgaccaaccagtccacaaccatctatgggccttgggagctggctccttgggtatcacagcactgggatcccctactggagaaaaggatttggcacaaagggagaggggaccgtaatgcactgggagaggccgtggcaaactcccttgctaaggtcttctcagagtgcccaggctccccagggtgggggcgtgcttcagggcccacatcctaaccccccgcctccccaacccctgggaggagggcccacattacctcgttgatgctgatttcggcctccacatactgcaggcccttctgcaggatggagatgagggcggcgggcggcactagggtcccattgatgttggactggctgatatggctctcgataccgaatgtgaacgccgagtgggaaaagcctgcagaagcacacgggaggtggggagggagggtggggttccatgagcaggcacccccggggggtcagacgcacctaacgagtacagggcaggggggtgcggtcatgctcacaggacgcccagcctctggtgtgggcggtgtcccagaggcagacggcagaccaaggtgtgactgcatggaacagctggtaaaaaagcagaggcttaaaaaaaaaaaaaaaagaaagtgcctttccagtgccattattattatttaaacaaagaaacaacttattaaaaaaaaattttttttggctaccaagtgccacaaatgggatcttagttcccggaccagggatcgaacccatggccccaccgcacccctgcactaggagctgaggagtcttaaccactggactgccagggaagtttccccctccccccatccccaaccctttccagcgttataaaagcaaaacagaggtcacagtctgccccattgtagacgttacagaagtcggatcccaggctctgtgtgcctgggttctgcatggctccttccagaagctcacgctgatgtctgctggcaggagccccctcccagcattcggggcaaatgaagctcaccaccaacaatgggaagaaacacgaggtggttctgtacccatcgcggggtaaccttgagtcgctttacagaactgggcatgtccctacccgacccttggctaagggctgaagggtccccccacaccctgcaaagcccctgcagcaccctcgtcaaatgcctctgcacgaacccactgacaacactgggcaggtgaaaggtggtcacagataaggcgtgcgcggagctgcctggatgaaaatatgcagggagaaagactgggccgctgactacacggaacaaatgaggcgtgtgtcaaaggagactgctgtaacaaacgaggccgtgtcaatgaaggaggctgcagggccaggaaggaggtttttgggggccttcgggttggggagggaatggtctgaggcataagtttcccttctgctctgtcacctgttggggggtcccgggcaccccacctccatcacttatgaacatcttgccatcaagactggctcatcctatgtgctggggtgggtagaggggttggggagggggagtctcatgggggaacctgcacactgcctccccacctcactttcctaggacacccccttcatcagccggatgatgggctgcccgcccccaggtcccccagcctccatccgggtcaacaaatacagccagcctctgcaatatacggaacagccagcaagatggcaatgttactccacgcaaggagagccctttctcttcctccacccctcctcctcatggcctgattattaagaatatatgcaataggcacttgattcatatttatttccagagtaatactgcagagcctgctattaacttcccgtgttatttctttacaagccggaaccacctacatgagactgtgcaggacagcaccttggagaccctctctccaacacagggtccctgtgctcctgttgctggaagggcatgagcagaggaaggccctttgcaaatcggccttctgtttttccttcttacttgttctctctctggccctcctttgccctaaccactgctgccacaacagaaacatccagccctgtcttcaccaaagaccttattctctgcatctccgccaccacacacagagccttaacctccaagctctctcatccagtttccggcaagggagcatgagaaaaaccccaggtcagcagacagagaaggacagagtgggtatcatcggctgagagacaccccaaccccatggactccctacgttctctggtccaccgtgccttctgtcttgtagaattcttcccgggtgaccgtctgacccactgactttttccatctccttatctgtgtctccaccagcagatcggacaggacgctggactgagagtcatctgcttccctcctggtatcgtcacaaggtccagagcaggaccacgcccgctggggtaataacaagacaccacccgccagccccgacagtgcccacagctgaccaggtcttgcttctaattgaataacggttcctaattcatgtaagccttcccaccaccaccttccctctctgctgactcagacggtgaaagaatctgcctgcaatgtgggaggacctgggttcgatccctgggttgggaacattccctggagaagggaatggcaacccactccagtattcttgcctggagaacctccacggacataggagccatgggggtcgcaaagagtcggacatgactaaacgactagactaaacgactaagcacgcacaggaccgcgtgcagtctctcccttgtacccttctcttctttttaggaatcaggtcccgagatcacccttggggtctgacacaggccacgcactcgctccaagcttgtggctggacacccgtctctcagcccaccacaaaattccacctggaaccttctgcagagcattcccccagggcatagcgtgggctatggccagggcccccgacaaataacatctcagcaactgacggagcagcggtcggagagaggtcacacttccggtgtatgtctttgtccgttccagctgctgtaagaaaatcccacagcctgcgtggtgtgagccacagacatttatttcccacagttctggaggctaaactccaaaatcaaggcactggcacgttcggtgtctgctgagaaccactttatggaccagacacatgactacgttttccctgtaacctcaggagcaagaaagctctgtggggcctcttacaagggcactaattgcattcacgaaggcccatcctcaggacctgatcaacacctaaagactgaacttccaaatattaactggggatagttgttcagttgctcatttgtgtctgactcttcgaacccaaggaccgctgcacaccaggctcctctgtccttcaccatctcccagagtttgcttaaactcacgtccactgagttagtgatgccatccaccatctcatccactgttgcctgcttctcctcctgccctcaatctttcccagcaacaaggtcttttccaatgagtcagttcttcacatcaagtggccaaaatattggagtttcagcatcagtccttccagcgaatactcagggttgattttcttcaggatggactggatggatccccttgccatccaagggactctcaagagtcgtctccaacaccacagttcaaaagcatcagttcttcggtgctcagccttctttatggtccaactctcatatccatacacgactacgggaaaaaccatggctttgactctatggacccgggttacgtttaaacatatgtattggggggaggcttgcaaaaatcagtctgcagtcaccttggaaatctgccatctctgggaaggatgctacccttggggtggcccagcctccaccaccatcagcgcccacccctcccaatcggactccagggctgctgagcacattcacttgcagtttgccggtcctatagcttctgtgccttaagcgtgttcagctttcagcacaccccttcttccacccaacaagacgtggaaacaaagaacggactatcagcgggtagagacggaaacgcttgggactggctcagtggcaggcgtgcaagttggtgaataggggcagaatagagagcttggtggcacctggatggccgatccaggtgacagctcagtgtgagtgccgttgctgtgcactggggcagccgctgaagctcggagacctgcccactacttagtaagaggcatccccgatgaagcactcctcaggaaggtttctcaaccgtcacatttgaaccaaactgttctttgttgtgaggagtgctatgtcctaggcgttacaagatttagcagcatctatgatctctacacactagatgcttgttttttgtttgggtttttttttttttttttggctgccctgcatgatcttagttcccaaaccagggaccgaatccatggccttggcagtggaggtgtggagttctaaccacggcaccaacagggacgtctcattacacaccaggtgttagcagcaagccttccgtagtcgtgacaaccgaaaacgtctaactaaccccagcacggcagagaaccaaaggcccacaggaagcacaccccccttagtgacacactcctcaggtctacatcacggacacatgtgcttcatttataaacaccacgggtggattgctgtcactgaaaagcaagccacagaattcaggtaacacatagtcatggggggcggggggagcgggtggacaaccaggtgaaggaagagtatttttgaagagaaaaaagggtataatcatgacaagatgggaaaggtttgcatgtgcttagtcactcagtccggagaaggcaatggcaccccactccagtactcttgcctggaaaatcccgtggacggaggagcctggtatagtgcagtccatggggtcgctaagagtcggacacgactgagcgacttcactttcctgcattggagaaggaaatggcaacccactccagtgttcttgcgtggagaatcccagggacgggggagcctggtgggctgccgtctatggggtcgcacagagtcggacacaactaaagtgacttagcagcagcagtcactcagtcgtgtccgactttttgtgacctccatggactgtagcccaccagtccacgggaatccctctgtccatgggattctccaggaaagtatagtggagtgggttgccatgccctcctttgggggatcttcccagcccagggagcaaacccatgtctcctgcattacaggcggattctttactgtctaatccaccaaggggaagatttatactacattttttataaggcacaacaagaccatcaagagttaagtatgcaccccatctgttcactgcagcatcattcacaatagtcaagacatgcaaacaacctagatacccatcaacaggtgaacagataaagacgacatggtacatacagaaaatggaatattactgagccaagaaaaagaatgaaacaatgccatttgcagcaatgtggatgtaactagacattatcatactaagtgaagtgagtcagagaaagacaaaaatcaaatgatagcacttatatatggagtctcttaaaaaaaaaaaagcactaatgtacttatttacaaaacagaaacagacacccatatttagaaaacaaacttgtggttactaaaggggaagggagggaagggataattagcagtctgggattaacagagacacagcaccatacacagaacagataaccaacaaggacctatggcatggcacagacaactatacttaataccgtgcaataacctataaagtaacagaagtttaaaaagaatataaagaaaaattaaataaaaaaattaaaagaatatcaattatatacatgtatggggcttccctggtggctcagttggtagagaagctgcccgcaatgcgggaggacctgggtttgatccctgggttcggaagatcccctggagaagggacaggtcgcccagtccagtatactggcctggagaatcccatagtccatggagtcgaaaagagttggacacaactgagcgactttcccttgtacttgctaaacagctggaactaacacacactgcagatctactatacttcaatagaaacaatacattgaaaaaaaaataagattttgaagagcactgttcttcccatttggggtgacaggtgctaggaaagcagcgtgtgcatggatgtcctcacctaccgctgctgctgctgctgctgctgctgctaagtcacttcagtcgtgtctgactctgtgcgaccccatagacggcagcccaccaggctcccccgtccctgggattctccaggcaagaacactggactgggttgccatttccttctccaatgcatgaaagtgaaagtgaaagtgaagtcgctcagtcgtagccaactcttagcgaccccatggactgcagcctaccaggctcctccatccacaggattttgcaggcaagagtactggactggggtgccattgccttctctgcctcacccactaggcaacccaaatagattgaaaaccaagcacatgctttgtttgctgctaaaccactaagccagacctctttgacaaacaacctcccaaagcattcgagctgtgcagtgaaccttcctgcccaactaatgtcggcctgattcatcagcagaacctgctaaagaatcactcactggcccttggaaacactgcctattcagtggagaaaggcagacacgtaatattcgggcagctgtgaggccggtggtccaccttcagctgaaacaaaggcacccgggctgggaagatcccctggagaggggaaaggctacccactccagtattggggcctggagaatcccgtcgactctatagtccatggggtcacaaagagttgcacatgaccgagcgtgtccaactcttaacattaaaggcagcatcaaaggcacttgtgtgaggcactaaatgcagtctttattaaggacttcatagacttaatattgggaatactgacagctatcaaaaccagtgggcacgatatgaaacttatccagaccacagatatgcagcccagggcccatgctgcctgtgtttataaatcaagttttattggcgtagtcactgcccatgggttgatatgctacctgtggctgctttcacagtgcaacggcagagcagagtcgctgcaagagacacagtatgggccgagagtgaaagtacaagtgtgcgttgctcagtagtgtctgactctttgcgactccaaggactgtaggccggcaggctcctctatccatggggattctccaggcaggaatactggagtggggttgccacgccctcctccagatgggccacaaagtcaaaaatatctaggatatgctcctttataggaaatgtgcgccaacctgactattccgtgtctattgtatatgcacactgatgtgtccctgtgttcagtagcttagctgtgtccacctcttttcgaccccacagactgtagcccgccaggctcctctgtccatgggattctcccatgcaagaatactgcagtgggtcccatttccttccccaaggtatcatcctgacccaaggatcgaacccgcatctctggcatctcctacactggcaggaggattctttaccaactgtgccacgttggaagcccatatatacacatatgtacaaatatatgcatgtatatctacgtgtacccacagacatatgtgcaaatgtatatatatatttatatacatatggacccgcagacacagaccacacgaaagcaagtgtcgcggtctaccctgtatccattcttccctttcttactgctatggactaaatgcctgtctctcaacattcatacactgaaaatgcactccttaaggtggtggtgtctacttttgaactgtggtgctggaaaagagtcttgagagtctcttggactgcaaggagatccaaccagtccatcctcaaggaaatcagtcctgggtgttcattggaaggactgatgttgaagctgaagctccaatcctttggccacctgatgcgaagagctgactcacttgaaaagaccctgatgctgggaaagattgtgggcaggaggagaaggggatgacagaggatgagatggttggatggcatcactgactcaatggacatgggtttgggtaaactctgggagctggtgatggacagggaggcctggagtgctgtggttcgtggggttgcaaagagtcggacacgactgagtgactgaactgaaaaagtggtggtgtcagggaggtgattaggacacgagggtggggtcctcacgaatagggttcgtgtccaaacatcgacacagatgaacactcagttcacgactgttatctgcaggacgacatacgactttcttcccacaacaccttcctggcctgtaagcagcccgcagtgaccccgccagcacctgggaattcctaccctccgtaatggtgagaaaaactgttggtgtttacgagccactcagcctgcagccttttgtcacagcagccgaaaaggccccaagacgcgcctcctgctctgtgtgattgacaaggtgacggtacccgtatctgctgttcaccgagcacttccctaagtgtcagctggcaatgttagagttttctaggtgctagacgggacccaagacccgtatgtatgtgtacccagagacaggttacccatggtaacctggctgatctgacacgaggaaccgaggactctctctccttctcaaaagcagtgaagagtctgcagacctgagcgtggccttggaagcctctcatttcctgcgctaagaagcttggccatgtctcactataccttgccaggctgcctttgccagaagcatatgtaccactttcccacaggacacctctgcttaagactctgctcccttggtctacaacggtggttcgtggttgtgaagtgtgggcacttctgtcggccagaggaagacatgggcaatatatggaaatacttttgcttgtcaccagaggctgtgccgctgtgcggtcgctgagtctttgaacagcattccacgcgtctgctacagttttttttttttacgaagcggggcaacagaggatgaggtggctggatggcatcatcgactcaaatggactctgagcaaactccgggagatggtgatggacagggaggcctggcgtgctgcggtctgtggggtcacagagatttggacacgactgagcgactgaacaatgacaactttctattcgaggagagaacacacttcaccagcttgggcctgccctcgttggcttccttctctgaccaacaaacaggtggaattaggtgatcgccaaggtccctttcaattctaaaattctatacagaggaaggaacggcctcattgtcccagttgaacccacaagatcttgatttacggaggtgagtgagtgacgacacacagagattaaggccaccaagaacccgtgacttgcagttcctgtgggaaggggccatgccacgctgccagggatacaagaaagcttatcgcatcaggggccacatcaccgagaaacagacaggacctagactacagctttatggaaacgagggtgagcaagcagctcagtgggaatgttgcccactggacagacgacagagttaagactgtgtgcttgggaggtctgtatcatgattttcacgagagccacctctgagaaagagagagcgagcctaccggctctggccatcagtccaaccctacaataatgaaggccaagcagccactggttggcacactcatccattccaactggctatgcggatggaagcccagttcaaaaatcactgcagatggtgactgcagccatgaaatgaaaagacgcttgctccttggaagaaaagccatgacaaaactagacagcgtatgaaaaaccagagatggtactttgccaacagaggtctcaatagtcaaaagctgtgatttttccaacagtcgtgtatggatgtgagagctggacaataaaaaaaggctgagcaccaaacaactgatgctttcgaactgtgtcgctggagaagattcttgagagtcccttggactgaaaggagatccaaccagtccatcctagaggagatcagttctgaatatccactggaaggactgatgctgaagctgaagcgccaatcctttggccacctgatgcaaagagcaaactcactggaaatgaccctgatgctgggggaagattgaaggcgggaggagaaggggaagatagaggatgagatggttggatggcatcaccgactcaaaggacatgagtttgagcaagctccgggagttggtgaaggacagggaagcctggcgtgctgcggtccatggggtcgccgagagtcggtcacgactgaacgactgaacaacaatcgggaatggcacctaacaatgggatcatctctggaaacaaaagcgtgggctgctacggtacatgcggtgtttccttgcttctggagcatccctgggcgcagagcagtcagcactctctgtccattcccctccgtccattctccctggatcagttttctgccacagaaccgcagcggcctgcaacacggcccgcgtccctcttggtgtgggggaccagcagactcgggtgtgggggcgttggcgagagtagaggtaacaaataaaaaggaagagaggctgagagcagtgaagcgggtgctgttgctgttcggtcactaagctgtgtctgactccctgtgacccggtggactgttacccaccaggctcctctgtccatgggatctcccagacagggataccggagtgggttgccatttctttctccaggggatcttcccaacccagggatcgaatccaagactcctatgctggcaggtggattctttaccactgagcccagttaagtgctgaatcactgaagttgctcagtcgtgccccagctctttgcgaccccatggactgcagcctaccaggctcctccatccatggaattttccaggcaagagtactggagtgggttgccatttccttctccagggaatctttccaacccagggatcgaacccaggtctcccacattacaggcagacgcttcaccgtctgagccaccagggaaacccagttaagtggcagctgggtccaggaaatcaaagagaagaaaacacacacacacacatgctgtaaacaggcaaggcgggagaagaagataccatcccctcatctttgggggctaatttatcatcagttcacttcttctttttggacgtgctgcatttgtctcgccagaggattaccttttctgcccgggaagccacacaaaaccgttaaccccagtttcattcatggtcaacagcacaaatattcactgagttgtttttgactctgcaatctgcaatcactgcacacgtgtgcatcgactttattgaatctaaaccaactcccccgccccaagacccagcccccactttctgtcctaattctctcctatcttgctggggaataacttgagcacaggcaggctgctctcagggctgcagacctactcccactgacctgccgtaacatggaatttggtataaaatggaactgcttggaaggcgcccaaaagacatagcaggaggacctcgttaaaaatagggttttgcttctaacatctggcctggaccccaggggtggtggctgagtgcgggcctgccaagatgtttaatatcccccaaacctgggagggctccaggaggagaagacattagtcaggttataaatgcagggtgctatttcctcccggggccgaccaactgtgaggggaggcagctcgcccacgaggggcgtcggggcacgctcccccagaccccagccctggcttccagccgccacacagagaagcaaggtttggggccagccccttttggccccatcaccccaagagcccttgaaccctcataaagtttggactcgctattttgttaattggggaattcaccatgaccgccaacacggagatttacgatggatgacaccgtaaaaattacaataatagaaatatacacacatacacacacacacatgacttagggggaggagaaacagagaaagcagtttggaagagagagggaacagtcttatcactagatgcttgatgcaagtgctgttcttacccgggggtaatttcctccagtccctcttacctctgtaaattgatttttcacacgcttaattcttgtggtcctagaattttacacccctgttctttcttaacctaacagagtatcagcacttctcccagtgactcattagtcccggccaccattctgcctgagatggtaaagagtttgcccccaaggcaggagacgcgggttggatccctgggtcgggaagatcctagaggtggacacggcaacactctccagtaatcctgcctggagactcctatggaaaaggaagcctggggggcttatagtccatggggtcgcaaagagtcactccaaagggcccactgaagcgacttagcattcacaccattccggtggagtctcagttttcacagagagtcacccctcatgtgcatggccttcagatttcattttcaaaaactagccaacgggcttcccgtcggacaccccggccttcacctgctgcccgtgacccagagtccctgtacttccttccccttcctcccatcctgcatccttggtacttcttgccaagatgcgtcttcacccctcaacagtgtaaccggcttttgctgagccttgtgacattaacgcaggcccttgatgatgttgcttgaaatgctctgcttcctttttcccctgaagagttctagttcaaatgcctccttctgtctgcacccctcccctgctcccctgtgaacaccgccaacctcacatttagcagcccttgtatcaagacgcctgtttattccctaggtggggagaaaaagacttcctcctctagtgtgtctccttccaaccctagcactagagcagtgccaccactcaacagggccagcgagagacatttggtagagctgaaaatcctgacaactgcttaatcggggcacatcagattcctggagagtgcctgtacctctgatggtacctgaaacaattttacaaagcacagggcgtacatattttattttattatgattttttaagatattttaaaatctaacaattattgttttaattgtgcattatgcaaacacaatagtagatgtatttgaaaatttagtgaaaagttaactgcttcaattttaaggaaaaatgttagtaatcattaacaaaatacttttaggtgtatgcagatatggcaaaatgatgtctctggtacaagagaaacaaggatgggaaatacatgtatagacaagggaggactacggacatgttcaaaggacaaggaagtaataaatactcaggatacaaacastgtgtccargttccgtggacaggaagaaacacatgtattgtatgggcagtctcagacaagagtctgcaagcygtatcctctgggccaaatctggcccctggcttgttattataaataaagttttattgaaacacagctactctcaatggtcyatgtacttttgatggctgcttttggaccacaaacacagaaatgacggttgcgatagagacaaaaacaataccttttaaagtaagattctgcaaactgatccaggggagagacctaaattatgggactacaggtcctcaggaaagcggtttttgatccatcggcttgtataaccctaagatgcaaacctaatgagaatattggaaaaggaacatcactgcctagcttccctcagtaaaacagacatgaacataccagaccagagaattaacaaaacggaattcagtgaagaggacaaaatgtccaggccacatgggatttatttcaggaatgcagacttaatgctgagaaagaaatccaccatcccagcacgttaagagtgaagaaaaatcatttaagtggaagattctggagccagagggtgtgctctgaattctagctctgtgacttcctggcagcatgacctgggcaagccgctatcttctcaacactttgggtcactcacctgtcaacgatggaggcagctgtatctacttcttgaaaataaaatgaaagcgttagttgctcagtcgtgtccaactctttgcaaccccatgggctgtagcctgccaggctcctctgtctgtgagattctccaggcaagaatactggagtgggttgccatgccctcccctccaggggatcttcccgacccagggatcgaacctgggtctcctacactgcaggcagactcttttttatcatctgagccaccagggaagcgccatctccatcttagggggaaacagtttgactcatcaagaactgagaatcatgcccagcacacagctcatgctcaataaatgggagctacagtcattcagacaaagcagcggaacagatggaaaccctgataagatatagggagtgcctggcagacagagacgcgaggatgagaggactcggcatgggggcggtcggaaagataccgtggacgagaaatagatgacagcgattctgacatttccacgtgaaacctggatgaggttacgggggtcagaggaagttcttctttgcaggggaggaactgtggtcaggaaatggcagtgctctcagtgtgggatcatgctgaaggatgcttgggaaatacacaactaacagcagtactgaaggcaacggagaggctggaggtggccccccccagactgggggatctgaggagacgctggggagagaccacccactcccacccccagacaggggatgagatgcgagttaaggatggaaacctacacttgcagaaagagcaccccactgcccgcagacctggatgcgggagggaaaggaaaaaggatgtcacctttgggatagtggcattttagacgtggacggaggagacgtcactagggctacagacagacactcgggagaaaaaaaggacagtgttgtgtctgacagtccagatgaccggctcgccgagtctggaaggaagaaagggagctctgattggcagaatgaagtacatcaaagctggacattttgaaggtcacattagaatttcagagcaggcagatcttaaatgtgatggtgtatgatctttggaaacaggcaccgtgctgagcacggttaattcatctcagctaaaaaaggaaacgggaaccatctgasgctttttcatatcgtccccaccctttaaaggaggaccaatggcctaggaagtcgtaaggacttcttcaacagcacctgaccttcctctgatggccatacgcagggaaataatttggctatattgagtaagacacacgctgaaacaagtgtaacctacacgcaggcttcctgggtggctcaagtggtaaagaacccacctgccaaggcaggagacccaggagacgcaggttcaatycctgagttgggaagattccctgcagaaggaagtggcaacccactccagtattctcgctcacaaaatcccaaggacagaggaacctggggggagctatagtccacgggatcgcgcaagaatcagacgtggctgggcgactaaacaacaacttacaaacactcggccgatttgcaagtgcttttctagcgcattgctttctcccaattctctgacaagtcaacaagcacaggaatggctaagcttgaatctgagctttgaacacatagacgacggagggcagcaaactcagaaaagatggcagaaccgcaaccgcatgggaaaccgggatggctgagaatctcggggatgtgtcgggagcaaccgcaccacggttccgttttgctacgcttcagtgggttatcatcacacccaaacagcagttcaggaagacacttttgtttttgaatgaagaggagctcgttggcaacgggcaccactcgaagagggtgtgggatgagatgaccccaacccagtgcagggtctttcgaaagccctaaggggcctaaggggagatggccccccgcagactgagctctgtcctgcaaaggaagcgtgggcaagctttgctgcgggaggtgtgtgcagccctgcccccacaaagctctcctgggggggtgctttctgtgtgagaggcgtgggtatcacattctccagttacttacccacccttacactttttacagtgagttttcaaacatgaggtaaaaacacacaagtgcacacatgaccgttacaccaccacacaccatggtgtccacctggaggggactggggctgggcggacagcctgggtctgcccaggtgctcctgccccttcgacaagagctgcaggtccacaaagaagcctggatggcaaagcttgggtggctaacaactcagcccctttgccatcggacatgctgctgggagaaccaggtgtcatcacactgctcagccaggaggggacacccccaagtctgtgcctggttctcctggactggaccccatgcacctttgcccctggctgactgtaacctgtctctttttaacttgggaggggggtgggggttgttcccaggtgctgctaggggtaaagaaccctcctgccaatgcaggagacttaagggatgcgggttagatccctgggtggggaggatcccctggaggaacgcaaggcagcccactcctggagaattcttgcctggagaattcccccagtccctagggttgccagagatgccgagagtcacttgaagtgacttggcacctacctacggattacatctcaatggagaaaaactgaagctatcaattcaaacaaagggttctcttggatactgtgtaaacaagaaagctgaacaaacagaatatacacccaactgtatacgattaagaggaatgtaacaaaaatggagcatcgactttgattaacgtgttggtcttttcgattgactttcttgagacgtaagtgccacctaacaaagtgtaagtttggggtatgcactgtgatgatctgatacatggatatacgctgtggaagaactacacaatacgctcagctcacatatgcagcacctcacatgattaacctttacttacttcactgagaacatttccaggatcttagcaaccgtcaagtacccaatataccactatgaagtaggttaaaatccgtatctttttgttgttataatccgtatctttttgttgtaatccactgcgagtgtaacagttactaaattctgtgaaagtcgctcagtcctatccaacacttggcgaccccatggactataccatgtaattctccaggcaggaatcctggagtgggtagcctatcccttatccaggggatcttaccaacccagggattgaacccaggtcttccgcattgcacgcagattctttgtttaccagctgagccacaacggaagcccaagaatactggagtgggtagcctatcccttctccagtggatcttccccgcccaggaatcgaaccgggggctcctgcattgcaggcagattctttagcaactgagctaccagggaagcccaaattctgtgactccttctagcaaatcatagagcctaggggtagtctaggagacagggatacatcaccaaagaactggaaacaaagaaatgagacatttctgtttcacctgactcatttccttgctccctccctccctttccgtcctcacagcctgcaggattgtagatccccaactggggatcgaaccccagtcccagcagtgaaaacaccaagtcctagccactggaccaccagggatttccctcacctgcttttaaattacttttatcctgtgctcgtgcggacaaaccattcaggtattccttacttattatctgatgagtattttcctgtatgctttctaaagcagaggctaccactgcctcacagaagtgcttgcctttaccgtttcataaggatcctctttttaaatggggacaacattagcaatgtaaaaatgccaccgcagtcagagaagtatttttagcaactcagctgaacaatgagccctgaacctgggtttaaattaagctagctgaaccattaaaatgccttcccgaatccggggtgggggaaacgtgtctagcataagaaggcagtccaagctcccaggaaacacagtggagcctacagggtgatctgtcagccgctagaattagagggcaatgctgctgaatcatttctggagacgcaattattcacgaatgttaagagtaacacccttttcctggttatctaatctcctcaccgggagtccagaaaaaaccaaaacccaaagaaaaacaaaacccagattgaagacagctactcccaattattttattatctgctggagaacagtattggcagaagttccccccttcttcaaggggaaaaatactctggaaaaattttataacaacagggaccgctaggaccactgttaagagtttcatttgctttctgcagaaagaatcaaaatgcagcccaagaagacgaaacagaagggagaaatgagaatctcgttacaactccactttgcaataaaaactaactgaagaaacacatctcacaactgaaggatgtgttatcagtggttctcaactaggggtggttttgccctcagaggaacatttggtaaaggtcagagacacctttggttgtcaccagggagggcgaggggagcagggcgttcctgtcatgcagtgggtggagtccagggatgcggtcttaagaacctacaatacagcgtagccacccagcccatacatcaacagtgctgaggttcaggaagccaggcctctgagaaaggtcgagcgtcttcagataatgcatgctgttctgaccattcagagtccctccaagagattcagttcagttgctcagtcgtgtccgactctttgcgaccccatgaactgcagaacaccaggcctccctgtctatcaccaactctcggagttcactcaaactcatgtccatcgcgtcggtgatgccatccaaccatctcatcctctgttgtccccttctcctcctgccctcaatcttttcccgcatcagggtcttttcaaatgagccagttcttcgagtcaggtggcaaaaggactgaagtttcagcttcggcatcagtccttccaatgaatattcaggactgatttcctttaggatggactggttggatctccttgcagtccaagggactctcaagagtcctctccaacaccacagctcaaaagcatcaatgcaagagattaagaccctcaaactctgcccacctaggagaaatctacaagtgaagaccatgaaggggatactgaaaacctggcaagacacaggaccaggaacgcagaagaacactgaagaacactattaaaatattgattgattttcggctgtgatgggtcttcggtgctgtgggcgggctttgctctggttgtggccagcagtggctactctcgagttgcagtgcctgagctcctcattgcagtggcttcttttgttgcaagagcatgggttcgaggggcacgtgggctcaggtcgtttcagctcatgggcttagtggctctccagcactggcatctttccacaccagagatcgaaccggggtcccctgaactggggggtggattcctatccatgatgccaccagggcaagtccctgaatatggacttttatccttctgctaacgatacagcctgtccaacgcctatggtgccaatcaaatgcagaacagcttctcacgacaatgttccaggaggaacgggccaggtgagcctggcctatgatgcacacttgagtctgctgaattaccaacgaagcaaaaatctggctctggatacacaaccaccactgttcaccttcagatgactctattatttttctgttgccaaaagccccagaatggctgtgtggctatccgcttctgagctaacatggtaacactcgaacccatcatacaaaagacactccatcagaaccgaggcctgagccttggctaacgacctgcaaaatcttaattaaaatgtctcggctgcatttgtattactgaccaccttctctgtctcagtcataggcaaagctctgcagaccatggaagcgtaaagtcatgatttctctggtcttttttattttagtaatttcaagatgatcataagctgtgaaaacagtggagtcatttcacctggccaccttcaacagtgacatcttctctcaccgtaggacagtgtcaaaagcagggaatgactttcaggtgatgggtcacctgactgcaaaccttcagcttccctcctttcagatcggtgtgtgagcatcacctaggcacacagggtctatgtgaaagctactccatcatgtctgactctttgtgaccccgtggactgtagcccaccaggctcctttgtccatgggattctccaggcaagaagactgcagtgggttgcctttctttctccagggaatcttctccaccccaggatcgaacccaggtctcctgcattggcaggcagttttttttttttgtttaaccactgagccacctgctgctgctgctgctgcgttgcttcagtagtgtccgactctgtgcgaccccacagacagcagtcaaccaggcttcctgtccctgggattctccaggcaacaacactggagtgggttgccatttccttctccaatgtatgaaagtgaaaagtgaaagtgaagtcactcagtcgtgtccgactctagcgaccccatgaactgcagcccaccagggtcctccgtccacgggattttccaggcaaaagtactggagtggggtgccattgccttctccgactgagccacctgggaagcacttattcagtgaattacacaatgtgagatcttccagggccagggatcaaacctgtgccacctgtatcggcaggtggatttttaatccctggactaccaatatgcataatttttacactgctcaggaacatttctgcagagtcatacctctgtgcgttcagaacagaaccatcctttggctctgtgcctggacttgcctgcggagagaaaatgccacctaaatcttactcggaatctgatgtgaggcggggcaagaaagggcatatgtgtgtgctacctggtcaggaaatctttctaagcattctcctaacagaagctccacgaaatgcatgaaaaaaatgccatctaccttccatttaaaacctcaagagtctggttatgtaacatgaattcacaaggataatacagaccacagagccatgtgcgtattatgtaaatcaaaggaaacatgcctgattctgcctggccagtcacatgagtgcagatatgggcttgaaaccagtccatcctaaaggaaatcaaccctgactacttactggaaggactgatactgaagctgaagcgccaacactttgaccacttgatgtgaagagcagactcactggaaaagaccctgatgctgggaaagatggaaggctggaggagaaggggacgacagaggatgagatggttggatggcatcactgactcaatggacatgagtttgagtaagctccgggagtcagcgatggacaggaaggcctggtgtgctgcagtccatggggtccgcaaagagctggacacgactgagcgactgaaccacaacagaaaatagctcctgtcccatgttgtaggcgcttctgtttgaaaaccaccagcgctcctcgcagaagacagcaggtatggctgtgctaaggtaggcaacagatatgcataatagattagcctcctgaccatgaaccacgacaaagcaggaagcatacttcactgtataaggagtttgccaatcttattcaaaggtgtttatttaattttttagtctttactcaatgtctcgccatgttgcttctgttctacgttctggggtttttggcctcgaggcacatggcatcttagctccccagccagggatcgcacccacaacccccacattggaaggtgaaggcttaaccactggagcaccagggaaggccctagaaggtcttttgttgttgttgttcagtcgctaagtcgaacccaagactcttttgagacgcccatggaccgcagcacgccaggcgcgaatgttgttaaatccccacaataagcagagaaatgcaccatcgtgttgatttctgatatatgcgaaacgaatttctgccatctgcgccgtaaccaaaggctgcttttattgtctcaagtgtttcaaatgacagccactggggacacccagcctggtaacacccgacaaaacattcgggaaacccaatctctcactcgtgtttctggttcgctgtcagagctaccaccacagtcgtcaaatggtttcgattaaaggttctatctgtttttcacggtgtgcagacgttggtcccaatgctagagggaggggaaatatgccccccaagccatgaacaccttgggtgaatctgcatgaatacccatgcaaatcaatgcacgtgcagtcttggctcctgtcttcccaggtggggcaatggtaatgcgcctgactgcccgtgcaggagactcagaggacatgggttcgatccctgggtggcaaagatcccctggaggagggcatggcaaaacactccagtattcttgcctgggaagtcccatggacagagtagcccggcgggctacagtgcatgggggtgcaaagagtcagacatgacttagggactaagcatatcaagttcagttcaatcactcagtcacatctgactctttgcgaccccatggactgcagcacaccaggcttccctgtccatcaccaactcccggagttactcaaactcatgtccatcaggttggtgatgccatccaaacatctcatcctctgtcgtccccttctcctcccaccttcgatctttcccagcatccgggtctttccccatgagtcaactcttcgcatcaggtggccaaaggatgggagtttaagcttcaacatcagtccttgcagtgcatactcaggactgatctcctttaggatggactggttggatctccttgcagtccaagggactctcaagagtcttctccaacaccacagttcaaaagcaccaattcttcggcactcagctttctttataaagaaactccagtttctttataaagttggatgtccaagtctcacatccatccgtgactactggaaacaccatagctttgactagatggacctttgttggcaaagtaatgtctctgctttttaatacgctgtctaggttggtcacagcttttcttccaaggagcaaacgtcttttaatttcatggctgcagtatctacctgctgtctacctagcattccacccagcgcctcccacccagttttctgcagaagggtgcactggctcaggagaagcaggcgggcacgtgtggaccctgtcccactcagcggatccatgggaaccacgaccccagtaacaggttcacagaggctaattccaaaccctgagaggaagcgttagcatttcatcagtattgtgcagatatatctgcaggagaccaatgtctttttacctattttccaatattccctgtgccccaggcactgattatcattagctctgtggcaggaatgaaatctataatataaaaccaccagcggctgtgaattgccttgctgttgagggagaccaaagcagacgcggtagattctgataaactgaacccagttttgtcacctggcccagcagtgcgcctcccagacagctgcccaagaaaaaggaaaaccccagaccacaaaagcctatgtgcaaatgcccccaggagccttagtcgtagcttggaaaccacccaaatgctcttccacaggacaaacgactaaccgccctgcatccataaaacagaatactgggccacgcttagtctcttaagtcatgtccaactctttgcgaccccatggaccatagcctgccaggctcctctgtccataggatgctccaggcaagaatactggagtgggttgccatgccctcctccaggggatcttcccaacccagggattgaacctgcatctcctgcattggcaggcgagttctttaccactgtggaagcccataggaaatagaagaccatgtagcatataaaggaagtactactgaagtatgcaccagcgttagccaacctcagatgcactgagtcagaaaagccagactctcaagggtacacgctgcagagattcacttctcagtcacaatcgcagaggcaacactccagtgccaggaagcagaccggtggcaccagcagctgcgatggggtgaggaggagactacaaagggggagtttttggtggaaccattccatatcttgtttgtgcaggtgtttgtatcactctgaacctctgtattctaaaaagggtcaattttctggcgtgaaaatgatactctatgtaaaaaaaaaaaaaaatggtagagatgaaggtgcagagggatggagaagctggacactgaccatgaagggaactgccaggctgggtggaagagacgctctgcctcaatcagcttcttacaaatcgtcaggatgacaaaagcgaccacatgattaacccccaagaggcagaggccgatggactcctagccctccaaagcggactgccgcaactcatgcaaaacataaaaattaaaagcacaactaagccactttgctgctggggctgctcaaaatctccagctgaactgaaaaaaaaaaaaaaaaagccttgcacactaaccgatcttctttatctggatgccaggaggagcaaccataaacaacagcatccagacagcaccttccacaggcacggcccttgctttagacaacagtgacgccatgaaatcccctctgctttagaggctgtggaatccacggcagggcggagggaggccagaggggaaccctagatgatgcgaattctgcctcttgagggcagaaaaggaagccccttttcaagatacttttcctgagtttgcagagatgtgatgtgcattttgtcgtaacactttcaggaaagtgttgcttctgcactgcgcctcccagctatctggtggggtggggtgcatgaggaatcaatgttctgatgggcaccgttttccctgcagccctgaacatcctgaccggcacatgtgctgcccgactgcaccagcccagcccctgcggtggcagagaacttgtgccttgaggtgggctggaatattctgggtctacccgctgcctggttgtacagccaacagcagacccccagagatccagaggtctgcctccctgcagggtcctccccacctgccatctcccaacctcctacacacacaccttctacacacatagcctggggggtgacacgactaaagacctaggaactttcaaacccctagtggagaagccacactgggaggagctgagtgagtcacctagcgtgacagagaatttatggatgatgggtttgatgctggtaaaggagagacagcacagatacttgatcaggagagtccttccttctccttcctgccttcttttccttccccttcctcccccttcctccccccttcctccctgtcccttccttcctcttcttttcttcattccctcttcttccttccccttctgccctcttccttcccttcccttccatttccttcccctcctttcagctagaaaggagacgttatgaggccaagaacatgctctgtccacttgatggctgctcagtaaattccctggtggctcagtcagtaaagactttgcctgcagtgcaggaggcctgggtttgatccctgggtcaggaagatcccctggagaagggaaaggctacccactccagtattctttcctggagaatccccatggccagaggagcctggtgggctacagcctatgtggttgcaaaacatcagacacagctgagcaactaaaccactacctaaccaaagcgtgaggatttgagataaatgttaaacaatcccacaatgaacatttaacaacattaggcatttcttctttggagtatgccctcgttggggtaccagaggcagttcccggacgcacacagtcttcagaggaacacacagtcatctcttaacttctcactcgatctatgggattctactcaaggctctgaaaaattaggacagccgctcagcagatgctacacacaatgggcattgatggaatacactgtgtctcccaaggctcctttctcagtcgcctggttacagaaagtttctcaacaaatgtgagacaaatgtaaacaaatcctaaaaaaggaagtctcgcttttctggggggagggattagacatttctgtgtttattcaaagaggccagcaacgctcttgccccggggggccctggggactgcccaccgggagaaacctttctctaggttacacaaaagttttctttcccaagtctctatttaccttcatgttcacagagggatggcaatttcaccttaagattgtttacacatttggactgcactttttcagcctatttttgtgcaagtgtttctaaagagaaagaatcctgaattccaagctggaagtcttcagggaggcagggtttggcacgtggaattttcatctgcagacatctctgggaatctatgagatcagtgctgggaggacacttggacgccaggtgagagatttcctggttaatttccgaggttaagttatcagagccgtgggttgatctccctagattgacgagggatgaggtggtacatattttgttccgctcatcatttttttcataggaaatatctagcaagagctaatccaaatggycwwatgwacatgtagaaagaaggtgtactcttcatgaataatgatagctagggtggacagcagaaacagakacctcaggcttgcgtggggagaggtttatttataacatgtggcatgccccacgtattttatcctttttgttcgaggatcagaatcgagccatttgggtcgcctgcaagactggcccatctcacccttctgggctgattctgaaagagtctggacccagggactcacacttggatcaacctcaatgatagcatgtgcggaagtgggatgtgagaactggcttctttctcgaataatcaatttgcagactacagaaaatgtgacacaaagtgaatcaagcttcaatatgcacagaaccgactgttatagcttgaactgtgtccgccacaacggcataggctggtagtcctagcccacagcwcggctcagaatgtgacggcgcttggaagagggtccctgcagatgwcatctgttaagacgaggttgtgctggaataccgtgagccccgtaatccactatgactgtgtctttacraaaaggagaagtcckacaaatgtggacagagagtgacacgcacacagggagaatgcctcgtgaaggkgaaggcaggtctcgggggacaaascgcctgtgawgcaaggaktgccawagacggtccakccgaasctggcakacaggccggagcakacmcycgtgaggcagacagtckcgggaagagagcaagccagctkgcaccctgctttggactgctgtcctccacggcgctggrayagtgcatttttgttcaagccgccccactgagttaccccacaccaattctgtacacggatctgtaaccagcccacaaacgctcgctctgtggccgggaccaagaacaccaaacaccccagccccatccatctatatatatatacacacacatacatacacacacacacacacatacatatacatacacacactatatgtatacaccagcccgcaaatgccctccctatggtggggactgagaacatccaatcaccccactccccatgtatatacaccatatgtatacattagcccacaaatgccctgcctatggtgggggctgagaacatccaatcmccccactccccgtgtatacataccatatgtatacaccagcccataaatatcctctctatggtgagggctaagaacaccaatcactctaccctctccctatatatacatacatatatatacacacacacacacacacacatacatatataccasaccacaaaagcccttcctatggtgggggccaagaatataaaatcaccccaccttcatatatatacaccagcccataagtatcctccctatggtgggcatcaagaacaccaaacaccccacctccatatacacacacacacacacacacacacacacacacagacacacacatttatatgtttgcttgtgtgtttagctgcccagctgtgtccgagatgctttgcgaccccatggactgcagtccgcctggcacctctgtccatggaattccccagataagaatagtggagtgggttgccatgccctcctccaggggatcttcctgacccaggaatggaactggggtctcctccactgcaggtggattctttaccagctgagctaccagggaagtccccatatgtatacaccagcccacagatgccctccctatggtgggggctgagaacactcagatcaccccacccctcatgtatatacaccacatgtatacagcagcccataagcaccctccctatggtgggagccaaggacaccctatcgccccaccctataatatatataccatacgtacacaccagcccacagatgccctccctatggtgggagctgagaacatgaaatcaccccaccccccacgtagatacaccagcccataaacaccctccctatggtgggagccaaggacaccctatcgccccaccctataagatatataccatacatacacaccagcccacagacgccctccctatggtgggggctgagaacgtgaaatcaccccaccccccacgtatatacaccagcccataaacaccctccctatggtgggagccaaggacacactatcgccccaccctataatatatataccatacatacacaccagcccacagatgccctccctatggtgggagctgagaacatgaaatcaccccaccccccacgtatatacaccagcccataaacagcctccctatggtgggagccaaggacaccctatcgccccaccctataatatatataccatacatacacaccagcccacagatgccctccctatggtgggagctgagaacatgaaatcaccccaccccccacgtatatacaccagcccataaacagcctccctatggtgsgaggtgagaacatgaaatcaccccaccccccaggtatatacaccatatgtgtaccccagcccataaacacccctcccctatggtcgggaccaagaacaccaaccgccccacccccatgtagatgcaccatacatacacaccagaccacaaacgccctcccgaggatggggacccagaacaccaaatcctccccgcccctcaccgcatctatcacaactgctcttgatcaaaatgggcttcccctgtggctcagctggtaaagaatctgcgtgcagtgcgggagacctgggttgaatcccttggtgggcaagatccccctggagaagggaaaggccacccactccagtattctggcctggaaaatcccatggaaagaggagcctggtcagctataggtgttcgggttacaaagactcagactgagtgactaacccttgatttcactttattagaggtacttccttggtggcttaagacggtaaagaatctgcttgcaatttaaagagacctgggttcaattcctcagctcgggaagatcccctggagaagcgaaagaccacccattccagtattctggcctggagaatcccatggaccgcatagtccacggggttgcaaagagtcgaacagcactgagtgacttcactttcccttcacttgatcttcattccaggctggcttcaggtgaacagaaatgtcagcacaaacaagaacacattccctcatcccgccccaagagcaggacggcccagccccttctccgcctcccttcagtcctctgaatgagggtgtccggagctctctcttccctatagaccgggttttccatccgtggttgctggccctgcaggtggcgtgttggaaggtgttttttttgccgctccgggcaggaagccggcttcttacctgactcctggagatatcggtacaccaggaagttcacctcgtcactggtaatgctcatcttagcctcacctcgcggggatgaggtctttacgaagcagcagccaccaccctctgtcggaaaaggcaagaggagaaagcggagagacgtttcagacactgcctgcgtgttcctcctcaaccgcatttctttcggaaaataactgatacatgtaatactcaggaggtaactaatgccccattcctgccatacgttctggtaacataaaggttttataattatctaaacatatatacacatacagatgaaaaatttcccctgtatttcctggtgacataaagcttttataattacctaaacatacaaacatacaagtgaaaagtatcccccaaaacacgagctgatgcgttttataatgctggcatggactcatctataaaccacattaacggagacaaaatcaggggcaccaaaaatgagaacaaacaccgcgacttgattcagtgaagatcctgcagcggttggtaccgagcttcaatggaaccccgagacagaaggtggctggtggcaccgactggcccttccctggacagacgaggaagattagagtcctgtcgacacccccaggttgggtgaccacgtggctctggtcacacctgtgggtctgcagcacagatgactcacagcaacctcccacgcatccaagttccatgctgaacctgacggtcaccgaaccgacagggtgcatgtggccccgacatacacagcgaggagctgagtcatcgacaaggagatatgacttctattaggaggcggggagtcagcaagcagccccgtggacagagtgcaacgacagcattcggttctgcggcagactggccaagaccatggcatcttcaaggatgaaccaagtgcttctcgggacccaggggcaggaagaccagcctgtgtgcccggactgtggactgagatggcctgtgtcatcggccaagcaggattcttacagagctcagaatgcttgcatgtgctaagtcgcttcagtcacgtccgaccctctgcgacccccatggactatatgtagccccagccaggctcctccgtccctgggattctccaggcaaggacactggagtgggttgccatgacctcctccagggaaatgcttgtggtgactgcaatgaagttaggggaaccccaaggcaatggttcagacctccaaggactgatgggttggaggtaaggcaatcatgtcctgagggcaccaagaacagagaacatggcactcggagcgtgggggggcttgaggctgcgccaggaggcctgatgctgactgcaccttcatccctaaggagctgtggaccacaggcctcagttttctacctatgccagctgcggatgctaagaaccgctgccctccccctcccccaggaaggtggtgaggatgacatgcttctacacacacttccagaccagggccctgagcaccacagagacacaggagggctcttaatcctactccgccccactgcaagcatcctcaaagcagagagaccaccaccgcccccaggaacacgcctccaaaaacaataacaaaaatgggttcttgattagtgacatggtgtttattttcttgcctgacccagttttgattcatgcatgtgtgtgctcagtcgtgcctggctctttgcaacccccatggactgtggcccgccaggctcctctgtccatgggatttcccaggcaagaataccggagcaggttgccattcccttctccagagttctcatcggaggcacctcccattaggtagatctgagaatctttgtggttgtgactgggcagaaaggaaatgaccgtcttcactgtgtggtaaaaaccacacattccctcactgttctggtttcagaagaaaaaatcagtgtgagctgatgacacgtctggagacttcctgcttaacatgagtgttccaacataaataaccactggggggctgaaatccatcaggaagctgaagggaagattwtttasggtatttttccttttttaataacttactttccaggtggcaaaaaactcttaataaccacagtcataaaacagttcaaaattcagttgcaagagaagtcgtgtctatgatgttatacttaaaatggagaaccaacagaacctgctgtccagcacagggaactctgctcaatcctctgtaataaccttatggtcaccagggggaaggatgggggaagggacagttagggagtttgggatggacatggacacactgctgtatttaacatggagaaccagcaaggacctgctgtccagcactgggaactctgctcaatgctctgtaataaccttatggtcaccagggggaaggatgggggaagggatagtcagggagtctgggatggacatggacacactgctgtgtttaacatggagaaccaacaaggacctgctggacagcacagggaactctgctcaatgtcatgtggcagcctggatgggaggggagtttgggggataagggatacatttaatgtacggctgagtccttttgccttctaccagcaactacggcaatgttgttaatcggctgcatcgcaataccaagtacaaagttaaaaaaatagtttttttttttttttttttttaaagggagtgggaaatggtgtctaagggtttggctgtattgcgtgtacagtatttaacatgctctcatactccaggaagggacaggaccgctccccaaacaacacaatctgacctgctctgtgatgatcttaaaggatgtaatgaccacagagccacttgagcgtcagctctgggagatgaagacagacagggcgagtgagtttccctgcccacgggcggggagcctggttcagaaacagcgatggaacaagcacacagtcactccagggtggggagcgtggtgttcagtgtgatatcatccatggcagaagtttctctcttagtaaatcggtgccgttagracacagtcttataacatcctctcatgcggccccttccgccgttctccagtgatggaagaaaaagtcggtctgaggtcttgtctctgctatgagtctgctggcctggacactggaagaatgcatcgcgtaaggtgatcttctggacaaggccctgggacttgattccacgctgagtaaggatgcaaagccagagctggaaaatctgctataaatgacctcattgcagcaaggcactttcacaaattctctgccaactgcaacatagtaggactcacctcctggcttcccttctccataatactgggggtagtttagccgatcagtcgtgtccaaccctatgtgaccccatggaatgtagcccaccaggctcctctgaccatgggattctccaggcaagaatactggagtgggctgccatttcgtcctccaggggatyttcccgactcagagagtgaacctgggtctcctctgctgcaggtggattctttaccgactgagccaccagggaagtccataacattgctctcttttcaaattgaaactgtgttttccagtggccagttatgagactagaagcttaatgtgtgtgtcaacatttacatataaatgctcttgtacacactgctggagactaaggtggggaggaagcaagcctgtttcatcacattacacacctgctcagcttgcattaactgaaacgaagccgccacgtaactgctggaaaacgactgcaaactttcgttgttgtccaaagacagtaatgagtactattcaataaatgatggcttttatggttggacttcagttattgcagaatgtacatacagaatagattggacagaaaacaagaaaatgtcaacagtggtttcaccttcttttcttcccccacagacttatcttcattttactaaagagtatttgcatgcatattatcctacagtgagaaaaacagtccccccaaaggttaagatttttcactaattgtatagttatatttatttacttgggagcccaatggtccaaaattataaacataatcaaagaactattctttcctcaccagagttcacatgcccagcagtggcatgcatgcgtgctagcttgcttcagcgtgtccgattctttgcaaccctattacctcagcccgccaggctcctctgtccatgggattctccaggcaagaatgctagagcgggcggccctgttctcctccaggggatcttcccaacccagggactgaaaccacgtctctgacgtctcctgcactggcaggcgggttcttaaccaccagtgccaccatctgaataagggaatgcagtaacctagtaagactgcagtcctcaaaaggattttcatgaccggcaatgacatccatgatacagtgctacgtgaaaaaaaaaaaaggaggctccaaatggaatgcgtgtgtgcctaagttttagtttgtacacacacacaaggcctcccccggtggctcagtgggtaagcaatctgctccccaacgaaggagacccaggttcgatccctgagtgggaaagatcccgtggagaaagaaatagcaatccattccagtattcttgcctagagaattccatggacagaggagcctggcaggctacagtccatggggttgcaaatagtcagacacgacttagcaagtaaaccacacaaaccacacacacacacacacgtacaaataatagataccctctaaaatgtaaacagtaaaataaaatgcaccagtctctccagggctgggacgatgctggacttaaatccgtgtacgtgtggcgtgccttttcggtattcccctaatttcctcctacaaacgtgaactgcagtcacattcattaggatgtaattaaaatcaggcagcgtcttcagaaatcagtatgagaacaacccagctaaggaggagagaaattggcagggttgagacagcaaggagaagggagcctcctcgggttagaaaaataatgacacgcttcacagaaatcaaaaaacacccccagggtgaggccttcagaaaggggcctttctgagcctatgtgccggcccggattcattagtggctggttggttagctttgttccgactctctgcacacaggcacgaagcatggagacgcgagaggacgggacgtcctggcccagacgtgcggccaagaccacgtgtccccctcccaagcagccatgggtccccgtctcgaaccctctgagctgcacaccctgcccaccccacacttctctgccccatcgggaggcaggtccagagggaaaccttcctaaaaaagttcaccccgatgatagcaagccactgctaagtaaggtctgcgtggcagagagacagacacacacgtaagtccgacagcaccgttatattttacagccttccgtccacgaggtgaaatgcaagctcaggtgcccaagctttcgcagctaagtgcctcgggaaatacagacaacttcaatgtaaaaatgccctttcccttctttgctggaggacgtgggtctgccagctaaggcttccgattagtccgtaaattaatttacagactttaattatctgagaccgaggggctggaatgacagtctctgcactaggccctctgacaaatcacaggaatatacacaggctggttacgaaagctctcagacaaagtgattttcagccacatatgcaagatttatcttggcctggatgattcccttttctgtggtaggtggagtcctaagtgaggtcatcgactcctataatgtggtctccagagttttgaaaaaacaaacaagcaaaaaaccctgtggctttgtctgtaaatataaaggcaggcgatgctttgccctctgagtcagcctgtgtctcaaaaaccttggtgctaaaatagggagatattgagggcaggaggagaagggggcgacagaggatgagatggctggatggcatcaccgactcaatggacatgagtctgagtaaactccgggagtcggcgatagacagggaggcctggcgtgctgcagtccatggggtctgcaaagagttgacacgactgagcaaccgaacaacaaaaacaaaagtatactgtgatgcttcagctttaaatgagcatggtgacccatgcctctattcttgcctgggaaatccctcggacagaggagcctggtgggctacagtccatgcggtctccaagagttggacatgagtgagcacgcatgcacacacaacaaccacacaccacataaaagaaaaatactcaaagcaccgtgaagccgagtatcccaagagtgccctttgacactgaaatcagcaagcagaaggcattcagttttgtcatctgacgctcggcaggtgaaattcccacagccagaggagtgcgcagatcaaaacacagaaagaaattctgatgcgaacagggaggggtcgtccgtctcggtactgcggacactcaggaccacatggatctctgtgacgggttccccgacgccctggaggatgacaaacaacatccctggtctccacccactgggtgtcaggagcaaacatcttcctcaaaggtctccagacgtgatcaaatgtcccctggagaaggctggttccagacgtccagagccacactgcccatggaaggccctgtaactcctcaagtccatgagcagctgcaggctgttggccaagcaaccacaacttagtaataaagtgtcagtcacttgaccgtgcctgactctttacgaccccacggactgtagcccgccaggttcctctgtccgtgtggattctccaggcaaggagaatcatgttcatggagtgcggttgccatgccctcctccaggggaatcgtcccaacccctggatcgtacccgtgtctcctgccctgcagtcggattctttatcatctgagccagcagggaaaccacaatgtggacgctgtcttaagctcgccatggacctcgcagagctgcatgtcagaggccttctactcgaaggtcacggtcagaagacggagaaataaaatgggtgtctttcttgaggtcaaggctggcgcaccgcagcccacaggcagattcacacgtcgcccaccttccgtaaggcttaggagctatccacggtgtctctgtttctaaatggctgcaaggaaaatcaaaacaatcctattttgccacccctgaaattgcgatgacattggaattccagcgcccatcggcgaagtattactgatgtcactcagccacactcgttcatctgtgtatgatcagtagcggttctcacagcgcaaaggcagaggaaagcaacggagaccccggggcccacaaagcatcagctttcaacatctactctctggcccttgatggaaaacgtctgccaacctcactcccctgaagattctcccctctctacattagaacacgcccgtcttgttgttttccagccgaaattcctgtctgggaacctgaaggtgatgccctaagaatgtggatgctcctcgggcataacccactggtggctctgaaatgaagagtcaagaagacaccacagccatcatctgctgtgtgtcagtcactttccgaggcgggacctgctgaaactaggctaagtcaaaagggaacagaccaaggtccatctggtcaaagccatggtttttccagtagtcctgtatgaatgggagagtcggaccatcaagaaggctgagcaccgaagaatcgatgctttagaactggggagctggagaagactcttgagaggagtcccttggactgcaaggagatccaaccagtccatcctaagggagatcagtcctgggtgttcattggaaggactgatggtgaagctgaagctccaatcctttggccacatgatgtgaagagctgactcactggaaaagaccctgatgctggaaagattaaaggcaggaggagaaggggacgacagaggaagagatggttggatggcatcaccgactcaatgaacgtgggtttgagtaaactctgggagatgatgaagaacagggaagcctggcgtgctgcagtccatggggttgcagagagtcacacacaacttggtgaatgaacagcaacaggaacagacatgagccctcactttctgacacacagcacccagcgcagagagaaagggctgtgatttacagatgactcccaagctcacagtcacggaggcaccatgggcactgtggcatgtgagcagcaatgcctttccccagcataatcccaaatgaacatccgcatgtcgaaatcgcacagggaatcatcaatggctacacaaatttccagtcacctgtgaagctaaacaccaatgcccaactcacagcaggaagtcaacgtgtgcgctcaattcaactcactcaaagctcatgaatactcaaagccctgaatactgaatgcagactcaatcaagacatggatgagcaacagaccttctctctcccctctgacattgaaactatgactcaactgcattttaaaaatactgactgatggtgcagacactttggaaaagctgggatgattcttccaaacgttaaacagagttaccatatgactgagcgattctcctctcaggtatatacccaagagaaaatgaaggtacacatccagataaaatttatacacgaatgttcacggcagcgctcttctgaacaactcagtaaaagtaaacacaactcaaatgtccatcagctgatgcatggataaacggcctacagtgatccatccatacaaatggaatattctttggccattaaaagactcatgccacaacctgaacacctgatgtcacagactcaattaaccttttctaatttttcattttttaaaaaaaaccaaactaccccttccagataattaagtaaaatattgaaattctaatacagctggcccttgaacaaaatggagtttggggagaaaatccccttatacctacagggggccctccatatccatggttcctcatctgtggattcactcacggactgtgcacagcggttaggtatttattggggggggggggggattaacgtataagtgggccggtgcagttcaaacccatgggccaacggtacatatttaccttgctacatgtttggtcttaaacaaaaataccacataaggttgttcctgtggttaaatttgggcctatcatcttacttctgattgcttctttttaaagcagtacagcaaaaaatcttccgcaaatttccatcttttatgtttttaaaataacaaaatttctatcagtgtttttttttttcattaaaacgaggaaagaaccagaaaacacacaaaaataaaggataaaatcaaactctaaaattccatcttccaaaatattaacagtgcttcttcttgatagagggtggagaggggaaccatcagctttaggttgagacgtgggcttcaactctggtttttcattttttggggcgtcacaaggctttcgggatcatagtttcctgaccagggatggaacccacgcctcctgtaatggaaagtgcagggtcctaaccactggaaccacaaggaactccctgggtttccattcttgatgtttgtgcatctatgattgtttggtttcttcgtctgggtataccttatcatctcactagatctatcgatagtaagctacatgtttttctaataactttcaaggttaagtaggaaatcaatttgcagatagacacaaatcatattaagaaactaacaaagcaaagaattaagatacaggagccaaataagatacagcctcatgccctcaattataatcaattttgctgcttaattttaattcataatggtgatgcaagggattgattttagaagctgtcatctgctccagaaaagtttaaaatagctggttccgtattcagcttcatgttcagtacagtacagtcgtatccaactctttgcgaccccatagactacagcacgccaggcctccctgtccatcaccaactcccagagcttgctcaaactcatgtgcatcgagtcggtgaggccatccaaccatctcatcctctgttgtccccttctcctcctgccttcaatctttcccagcatcagagtctttcccaatgagtcagttcttcaaatcaggtggccaaagtattggactttcagcttcagcatcaggccttccaatgagtattcgggactgattttctttaggatggactggttgcatctccttgcagtccaagggactctcaagagtcttctccaccaccacagttccaaagcatccattctttggtgctcagctttctttatggttcaactctcacatccatacatgactactagaaacaccatagctttgactagacggacctttgttggcaaagtaatgtctgtactttttaatatgctgtctaggttggtcacagcttttatcccatggagcaagcctcttttaatttcatggctgcagtgaccatctgcagtgattttggagcccaagaaaataaagtctctcactgtttccactgtttccccacctatttgccatgaagtgatgggaccggatgctgtgaccttcattttctgaatgttgagttttaagccaactttttcactctcctctttcactttcatcaagaggctctttagttcttcactttctgccataagggtggtgtcatctgtgtatctaaggttattaatatttctcccagcaatcttgattccagcttctgcttcatccagcctggcatttcgcatgatgtaatctgcatataagtcaaataagcagggtgacaacatacagccttgaggtactcgttcccagtttggaaccagtccattgttccatgtccggttttaagtgtccgcttcttgacctgtatacagatttctcaggagccaggtatggtggtctggaattcccatctctttaagaatttcccaaaattcttagaaggtaaaaaagcccagttcatcttaaagaatcaaggaagcagcacatcattatttaataatgctttgtctattaccttcatggagaaggaaatggcaacctactccagtatttgtgcctggagaatcccatggacagaggagcctggtgggctacagtccactgggtcgcaaagagtcagacatgacttggcgattaaacaacaattaccttgaacttcagcaagaagaaagacctcagacatgtatcattttaaaacagaaggtgttcagttttggcaaaatgcagttctgtttgaaacgaatttcaggcggagaacaaacatcaaaggcaggggtgtacctttagggtgacttcatgtgtagacctcggaaccagtgggctccccagcacccgatcataggaactgccaagtgtctgctgggacctgcctggcgagaaacaaggagattgaaaaaaaggaaaaccacaacaacaatcaaaatgaacctgaaaattctttgggaattgttcacctttgatctctcctgttcttcttttcctttgtcatctcaaacacgttttactgtttcataccctctatcttcaacttttacccaaactgtgcagttttatttgcaaagtaaaaagggaagtaattaaaatcagccatcagcattatggtacatgcagagctttacaaggaaaacgtctcaaagtaaataacattggttagagtgcagacaagcattctgctatcattcaatgataatgaacgccctcttgaaacatctttgttttagggagtgtatcacataaaatcagtaaattaaaccgtacacaccctaaagagaagaaagatgcttcacagaaagttgatagacagcgcttgggccaatgttaccatcatcacgccatgcaagtttgcagccatgcactcagttatgtgctcatgcaaacagggaaacgttagaatcgtttctcccccccttctttcggcttcctaaaataaagcaaaaggaagcactgactaccaggtctcaagttttccgctgttattctcctacatttccatttcagagcagttctgtatttgcacagatttctaaagaagccaggggtatatctcacaagataacccttcatatcacaaacttgcttttctcagtcaacataaatagaaagcagtcaaccagcatctatttaaggatatgtacctccttccttacaaaatgtctttggttgtgagtgagtccaggctcctacaggatttaaattatccacacattcaaaaactgaggaaattaaatgactaccactttctgtaaattcttgttcaatagtcttaagtgatataaaaaggtgccaggtttaatgtaattcaaattcagaagcagggctatgaatgaagctgtttcttattagtgaattaatcagaacctcttgaaacgtttttaaggaaattcaaggaaattattcccactgagctatgtttatagttacactgttcaaagaaaggagattaaaaagattctataagcaggttcataggctgttaagtttcattacactatatttttaaaacatcataaggatctgtaagatgaaagtaataaccagaacaactgaaaaaaactctaccactgaaggaaaataagatttaaacaaaactctgcagttgtttgaatagacatttaacggcattagactggagcagggacaagggaatatggacttttcttataaaataaaaatgcatttcattatgaaataaattctgtagtagagatgaaataggacaatactcctgagagtaacctgacgcagaaagcaattgactattttcaggactgtgttgtaaacccaaaatagccttctgatgagactgctaaataccgtaaagtatcaaaatatgagcagatgtcaccacaataagccccaaattgcttgaaaattaaaacattaaaatccacaataattcaattaggcaattaatatataaagagctcttactacaagctgttcaggaaaaaaaaaaagcttcagtatttagtatttggacctattctttgtccaaaacagatggtaactcacaaattgaaggttatctttgatataagttgttaaattcaataagagtaattaacatcatctgtgtgttagaaacacacaaaacatgtgaaatatgactttggttaatgacattctggttatgcaacatgacaaattaattagtttactgctaaaaataagaagttaaagtgagaaatctgaccttccaataccttgtcattcgtgattttcccattacgaacaaccagagagaaccactacccataagccgtgctggcaagaattcatgggactgaaaaatatttgtgggacatgcaaatgattttaacaaatgcaatatattccttaattctcaaaggtaaacacacaacgaaagtgacgtgagtccctcagtcacgtccgattctttgtgaccccacagactgcagcctgccaagctcctctgtccatggaattctccaggtaagaaacctgcagtgggttgccattcccttctccaggggatcttcccaacccagggactgaacccgggtctcctgcatggtagacagattctttaccgactgagccaagtaacccgtaataagaatatggaagcatcgtggcacgtccacagtgttaacacaggtgctttcaaccaggggtggtgtgtcctctgccccagggcactctaggatattaggcaatgtctggaaacatttttcagtgtcacaactggaggagggacacgactggcatagcattaagcatctacaactcgcagaacagtcttccttccacacagtagacagatcaggcccccaaatgctactaatacccagaaactatattttaatgggacagactttagtgttacataggttagactagggaaacagagtctaagatggtagtcagaagaagggacaactacaaggaccacacgcagggcagacaaaggaaaggaaaaagcccatgaaaaggaaaacctgaggcctggaacaagaacctctggagggggaagaggcctaacacacccctttccctgactggcttgaccatacaccctaattgcattcccttccctgattggttgcagatacaagccctattttgcaaagggaaatagccaatggagcccgaggaaccgccaaggggaagagaaaaatgtatacaaagggaaacctaaaaatgacaccagaccactccaacgggtgtgggtccactctcctgtctcaagactgtcctgcgtgtggcttgcttaataaatcctgcctgcttgagctgtgtcggtctgtcgttttaattctttcctacgaagagacaagaatggagggggaaaaacctacttgccagtagactaagtagacacttttcttcgtgacttctgccttattcgtacggtgccatattagaagtacacatgggccttcgattcagaaaggaacacccacgggacactttagcatagactatcaaaatctaaaacgggttaccgaggtcaatcacggattctgtatagagaaaatggttcactaagagacacaaaagcatcagatatccaactgcaaaatagttcaaggcattcagtgaattaagagcatgggaataacaatgaattctgataagtgggggcttccgttgtagggggcacatttcctggtaaggggactaggaccccatatgcttcgaagcatagccaaaaaaaaaaaaaaaagaattctgataaagaaaaaatatttaagagcaatgaagcaaaataataactattacagagagaaagtctctggaaaaataggaataccagaacattcaaggttcagcagataatcccatcacttaatgaggtacaaatgaaagcaagaagaaactgcatcgttcatttgaaagaagaatatccatgtttgcaaaaaaagagaaatggctttaacaaaattgaaatttgagtggaagggcttgaaaatcaggtataaaagattataagagaggaaatttataggcttatccttttatgtaaatacaagtttagcaaaatgttgaaaatttctgatgctttgtaatgaatacttggaagtccattattctcttcatttcttagtatgtttgaaatctgccagagtaaaatggtttttttttttaatttacataaaaagatctgtgaacttattgatttgcccttgttacttatgaggctgtaagctactttgaaagcaagaacaatgccttctaccatctctttatacttcactgtggctaacggactaccttacattcagggataaacagctcatcagggtctgttgaaatgttaaaaactaaatgtcttttttggggggagctatggaaaatgccatggatggaggagcctgatgggctgcagtccatgaggtcgctaagagtcggacatgactgagcaacttcactttcacttttcactttcatgcattggagaagaaaatggcaacccactccagtgttcttgactggagaatcccagggacaggggagcctggtgggctgccatctacggggtcgcacagagtcggacacgactgaagagacttagctgcagcagcagcagatacagttaagtgtcttctagctctgagattccatggtatcaagtttcaatttatctgcttatgcttaactcttaactcaccaagataccaaaattacctggccaggtgatcaagtcacatgttaagtctttcccaaagcacaagtgaagagattccaagtttggcttgttttttgcaatcacaaaccagcaaattaaagagagaaggaagcaacctttacaacactgtcaggggtctaacaccctgatttcaagcagtctattttgtctgagttccctccaatagtcacttaaacgtccataattaatgagaaagtagaaaagagcctcccagaggaaagggccagggaactggagctttgagaaaagactaacatgtgacttgatgactgctgcactggaaaaccaagaggaaagcggaagtgaactcacaagatttgttgcacaagtaaaggaaaccgtaaacgaaacaaaaagaaaacctacaggctgggttgcaattcaacaggactccgtttccaaaacacacgaatagctcatacaactcagtaacagaaaaccaaacaactccatcaaaaaaactggcagaagagcaaagtagatactcctccaaagaagacatacagatagccaatgagcacctgagaagatgctcgatgttgctcatcactggacaaattcaaatcaaaactacaatgatgcatcacctcacacaggtccgcatggccatcatcaagaagtctacagataatagatgctggagagggtgtgcagaaaagggaactctcgtacgctgttggcagaaatgcatttggtatggcccctatgaagaatggtaaggaggccccttaaaaaaactcaaagtacaattaccataggattcaacaatcccactcctggttatgccgctgctgctgctaagtcgtttcaggcgtgtccaactctgtgcgaccccacagatggcagcctgggtatatatctagagaaaaccctaattctaagagatacatgcacccacatattcacagaagcactttgcacaatagctaggatgtggaagcagcctacatgttcaccgacagatgaaatgctaaaggtgtgggatgttgtgtttagtcgatatattgtatccgactcttctgtgaccctatggactgtagcccaccaggctcctttgtccatgggattctccaagcaagaatacttgagtgcgctgccatttcctcctgcaagggatctttctgacccggggatcaaatccatgtcaccagcatttgcaggtggtttctttaccaatgagccacctgggaagcccctaaggtgtggtgtgtgtgtatatatatatatgtgtgtgtgtgtgtgtctcagttttatctaactctttgtaaccccgtacactggggtagcctgccaggctcctctgtccatgggattctccaggcaagaatacgatagtgggttgccatgcccttctccaatatatctatatccatatccatctacacatatatatacacatatacatatatataatggaatattactcagacataaaaaaggatgaaataatactattttcaataacatgaatggacctagaggttattgtatcaagtcagacaaaggcaaactcatcacttatacgtggaacctaaaatatgacacaaacgagcttagttacaaaccagaaagagactcatagacatagaaaacagatttatggttaccaagggagattggggaggggataagctaggactttgggactagcagacacatgctactatacataaatcagataaaaaacaaggtctttccgtgaagtacagggaactacagttaatatcatataataagctataatgaaaaagaaactaaccataaaaacggtttatacatagctgcatctactgtggatctgttctaagtgccttgaagttatgtaatcctgctaataaataatgacagaatccttcctaccagaatgtcacgtttccaacgaggatcccaggtacccccacgagcagtgtcagactaggggttcacacacagcaagtcatgactttcttgtcatgatgccttcctggctccaagctgcaggcagcagaaattttgtgaattgctgtccttctctaggggcttccctggtggatcagctggtaaaaaatccacctgcgatgcgggagacctgggttccattcctgggttgggaagaccccctggagaagggaacggctacccacttcagtattctgccctggagaattgcacagactgtatagtccatggggttgcaaagagtcggacataactgattgactttcactttcactttccttctctagcttgtctgtgtacctaggtattcacctagaaaatgacactccatgctcaggatataactttcccaaactttaacttttctgacacatgctgagatgttcctgacgacttcctgacagattttgaatacaccagttgctagacattctaataaagaggttctcctgctggaagactatagattccccagaagaaatatgaaaccaacgtgcatttctctagactctggaaggctgcaagctaaagatggcagcgcttgacaaaaattaatgcaagtctgccaagatggtaatgacttacaaaaataaacttgacttttaacatcataatagtggcaagaagtagggcagttctacacgcgcctttacccactgggaattaaaatcaggccatgatcctcacgtctcttttcactcacccaactaggaacacgggtaccgtgaggtgagaggtcatggtgcattaagcggttcctcatctcctaccatcacattcctctttgaacaccattcatttatgggatgctctttatttagatggattagttattttttgtcactatactaaaagggtacccaattctctttatacgtaaactggcagacaagagtaaagggatctgttctctgttaagagttctatttatgacacacacacaaagattctgggttgttttgttcatgataaacactaattacgtgcatgaatttcaaggggaagaaaagggaaggaaggaagaggaaggaaggattgggaaggaaggggaaggaattggaaggaaggaagggggaggaaggggaaggaattggaaggaaggaaggggaaggaaggggaaggaaggaaggaaggggaaggaaggggaagggaggaagaggaaggaaggggaagggaggaaggggaaggaaggggaaggaataggaaggaaggggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagggagaagggaagaagggttgaaggatgacgggggaggaaggcatgaatgtgggatgaactggacgaatggtagaagaaagggaaggaaatgaatgaaggaaggggatgaagaaaggggaagggagaggaaggaagggaaggaggggaaggaaggtagttgaatgtagggaaggaagaggaaggaaggggaggaaggaagggcatggaagtggaggaaggaacagaagaattgaaggaaggaaggggaaggaaagggaaggagggtgaggaggaaggggaaggaaggtagaggaaggatggggatgggaggaaagggaaggaagaggaaggaaggggaaggaagggagaggaaggaaggggaaggaagaggaaggaaggggaagggagaggaaggaaggggaaggaagaggaaggaaggggaaggaagagcaaggaaggggatggatgggggaggaaggaagggcatggaggcgggaggaaggaacaggaaggaattggaaggaaggaaggggaaggaaaggggatgaaggaaggggaaggaagggaacaagggggaggaagggcgaagcttggtgttttgaatcctcgattaaaaggcccaggcatttcaacaaaggcaccctagtacacacgagtgcatctcacacgcagagagagcaggagtaacgctgcagggtccttcacagggaacacacttgtatgtctctattatttctctaactaggctactctgctgacaatcctccccttttctgaaaccaagtttaaaaatcgggagatagtcaggatatgacgaccgaggagggaaaagttctagactggtaaagcaggggtgagcacactttctctgcaggaatcttggatccttgacacccaaactgagcctgctacaactgctcacgtctgctttggctctaaaacagctgcagacggtacataaatgggtgttggatgtgcttcaacacaattaaaaaaaaaatagattgcaggtggggttggcctataggcaggagtctgccgatccctgcctgagggcagtatcagtgaaagtcattcagtcgtctctgactctttgcgaccccatggactacacagtccatgagattctccaggccagaatactggagtgggtagcctttcccttctccagaggatcttcccaaaccagggatcgaacccaggtctcccacattgcaggcgaattctttaccagctgagtcacaagggaagcccaagaatactggagtggatagcctttcccttctccagcggatcttcccgacccaggaatcgaaccagggtctcctgaattgcaggcagattcttcacccactgagctgtcagggaagcccaaggcaatatcatgctcactttattaaggtgtgtactccaaacttcttgtgtgcatgtgcgcagagtcacttcacttgggtctgactctctgcgaccccatggactgtagcccgccaggctcctccatccatgggattctccaggcaagaacactgcactgggttgccatgccctcctccaggggatcttcccgacctggggattgaacccacgtctcttatgtctcctgcactggcagatgggttctttatgtcccctgggaagatgacccatattacattaaaacagacaaccttctaatatcctcagggtgacttcagacaattaacatatgacagaagtggtctattcgagaagtaaaacaagtctctgccttgtttctgggaatgaccacagtggagggggtgcagttggagtgaggggtgtatttcagcccaggttcttggtctctgggacacacacacacacacccatacacacacacacatgtacacctgccatgggagctttcatgacactagctcaaatgccattctaggttaagatgatgctctcagaacccaagtgcaaagtctcatgaaacttttcagttctttcacggatcgcataagcgtgaaactgaacagaataaatctcatcagtgtggagatgacatctctttttttccccctccctcgttaaacacgatttttaacaggtttcgtttttagactagttttagatttccagccaaagtgacaggaaggtacacagatttcctgtatgccatttgcccctacacaggctcaatttccctccttatctaggtcccccaccagagggtccatctgttacaactgatgaacccacactgacattatcacctgaagcccatagtttatatcaggctgtggcaccttctatgggttttgacaaaagacataaacacacctgagccccccaggtggcgctcgttgtaaagaaccttcgcacttcaatgcaggagacgtaagagacacaggttcgatccctgggccaggaagatcccccggagaagggcatggcaacccactgcagtgttcttgcatggagaagcccatggacagggagcctgttgggctatagtccatggggtcgcaaggagtcgggcaggactgaacgactttcactcactcactcaagaaaaccgaggcttagctgactgacggggttgcaagaagtcggacatgactgaagggacttagtacgcacacacgcataaagacacgtgtccaccactaaaccaccatcaagaatacttccactgtcctaaaagttctccgtgctccaggggctcatctcagcactgaagatggtgctgcaaagagtcagacacaactaagtgattgaactgaactaatattccactgtctagacgcaacattcaaatatttatccactcacatataaaaggacatcatggtttctcccaagttttggcaatgatgaatcaaagctctataaacatacgcgtgcaggttttttcctttgagtacatttttttaaaaatgccattaaaatgatataagtaaggacttgcctggtggtccagtggctaagtctccacgctcacactgcaggggcctgggctcgatccccggtcaaggaattagatccaacatgtgcaactaacgattctggatgatgcaatgaagcctaaagacccacacagttttgcactaagacctgagcagccaaataaatcaacatactttttaaagaattatctaattaacagatgttcaggggaaagcttttaagaggttctcagttgtgggcaactgtgtccccgtagggtcatcagtcaatgtccggagatattcctcagtgtcacaactccggggagggtctctggcatgcaaggggtagaggtcggacaagacattcccacgtgagaaaggattcttgggcccccaacgtaactagtgctgaggctgagaaaactctgctgctgctgttcagccactcagtcgtgtcagactctttgcgaccctgtggactgtagcctgccaggctcctctgtccgtgggattctccaggcaagaatactggagtgggttgccacgccttcctccaggggatcttactgagctggggatcgaacccatgtctcttacgtctcctgcattgccaggcgggaagcgccactagcgccaccagggaagccctgagaagccctggtatgaggataaaatgctaactctccctaggtccactccgtctcacctcgcagagactgcccctatttcccagctccccctacagtcctccaagtgcccaccgtgtggtactcttccttttaacacaaacgtgatctgctaactatcatccctgaatctgagtcatgagaggtgcgtgtcactggttttcatcacgaaaacctttcaacagcaaggctgctcagtacctgcctacataaaataactcgtcgcccacctgttctcgtagaaagtgtacattttatcaagtccctctaggtccatgatatgacttaaccgtcacaacaatccttttgttattcccactgcctagctgtaaaaggccaagttctagagagtttaaatgaacgtaacagttcagactggtttcacagctaggaaatgttcaaatttgtttgttgttgctcagttgctcagttgtgtccgattctttgcgaccccatgggctgcagcactccaggcctctctgttcatcatcaactcccggagtttactcaaactcatgtccattgagttagtgatgccttccaaccatctcatcctctgttgccccctcctcctcttgccctcaatccttcccagcatcaggatgttttcaaatgagttggctctttgcatcaggtggccaaattactggagcttcagcatcactccttccaatgaatactcagagatgatttcctttaggatggactggttggatctccttgcagtacaagggactctcaagagtcttctccaacaccacagttcaaaagcagccattctttggcgctcagctttatggtctaaatctcacatccatacatgactactggaaaaatcatagctttgactagatggacctttgttggcaaagtgatgtttttgctttttaatgttctgtctaggttggtcatagcttttcttccaatgagcaagcgtcttttcatttcatggctgcagtcatcatctgcagtgattttggagcccaagaaaataaagtctgtcactgtttccattgtttctccatctatttgccatgcagtgataggaccggaagccatgaccttagttttttgaatgttgatttttaagccagcttttccactgtcctctttcactttcatcaaggggctctttagttcctcttcacttgctgccataagggtggtgtcatctgcatatctgaggttattgatatttctcccggcaatctttgagtccagcttgtgcttcgcccagcccagcgtttctcatgatgtactctgcatgtaagttaaatatgcagggtgacaattcatagccttgatgtgttcctctcccaatttggaaccagtccgttgtttcaagttctgttcaacttgtccagttcttgacctgcatacagatttctcaggaggcaggtaaggtggtctggtattccgatcactttaagaattttccatagtttgttgtgatccacacagtcaaaggcttcagtgtggtcaatgaaacaaaagtagacgttttcctggaattatcttgctttttctattatccaacagatattggcaatttgatctcacactcctgagctgatctgaaatgtattcagtcatttgcactgtgtatatgtgtgttagtcgcttagttgtttccgactctttgcgaccccgtggactgtagctcaccaggctcctctacccatgggattctccaggcaagaatactggagtgggtagccattcccttctccagggaatctagacgactcagggatcgaaccggggtctcctgtaccgcaggcagattcttcacagcctgagtcaccagggaagccctcgtttgcactttatctacaagcaactacgctctattcagttttggattttctgtgatttttttttttttttcccttcaccggaccgccttccaggtttccgcagttagccttgacatctagaagatgaaagccagaacgggagtgagtcatcctggggctttgcattccattcacgtgcgcatctctcccatggcgtcaaactcagatcaacagcgaatgtcttatttatagcgatggtttatttatagatgagcaggatgaccaaaagcacccttatgcctccaagaaagcagcagcgtgtgcagctggaccaggaaaccaagtgcgggaaaaggcagttccttcctgattcatggaatgcagttctttggagataaaacacaccctatttgtcattaaaattctttgtttctgagtgcgtctgccttcaagatgttaacgcttctcttgttccatcattggtacgggagacttgtgaaatcacaagcaaccattattattagtgtcatgactggggacttgtctgggagtttagtggttaagacatggcactttcacagcaaggggcatgagtctgagccctaactgggggactaagatactaaatgcctcgaggtgaagccgaaatacacacagacatatatatatatacacgtatgtacacgtgtattcatacacatacatatatatacacatgtacgcacacattgatgactgtaaaatcatagctaaatatgtacatcaggcctcatttcatgcaactacagctattgaatatgaattttccaaactagaaaaggagagctgggaattccgtggtctctgtgctttcaatgtcaagggtctgggtttgattgctggtcacgtaattaagatcccataagcccttgtggtgcagtgaaacaaaaagaaaagaaaaatgtcacccaccatttttttaaagtcttttttgattttgttacagtatggcttctgctttatgttttgggtttttaggcctgtggggtcttaagcttccccgaccagggatcgaatgcacacctcctatactggaaggcaaagtctcaatcactggatctccagggaagtcccggcccaccttttttagaattctgattcctcactgccctttcctcctacctgacggctgctaaccaaaacagactcacgtgtgagcgccagtcagtcagtcctccaaagccagggacgaacaggacatgtgggttggtctgtctcggctaagatgactaaaacagtttgagagtttgtttctgcctagctttcaagacacaagcttgccttcaggtgcaggggctgcaggaggcagtttctagtacgggctccaatatcactgggggaggcgactgcagtcatgaaattaaaagacacttgttccttgggagaaaagctacgaccaacccagacagtggcttcagaagcagagacatcactttgttgacaaaggtccgtctagtcaaagctatggtttttccagtagtcatgtatggatgtgagagttggaccataaagaaggctgagtgccaaagaatggatgcttttgaactgtggtgttgaagaagactcttgagagtcccttgtactgcaaggagatccaacccgtccatcctaaaggaaatcggtcctgaatattcataagaaaaactgatgctgaagctcggatactctgtccacctgatatgaaaaaacaccctgatactgggaaagactgaaggtgggaggagaaggggacgacagaggatgagatggaccgatggcatcaccgactcaacggacatgagtttgaataaactccaggaattggtgatggacaggaaggcctgttgtgctgcactccatggggtcgcaaacagttggacatgacctagcgactcaacggacaacagcaagtatgtgattatttggagaaacaaacacttgagacaattcgtttgaagcaagaccaagaaatctatcagttaagtaaagcctcctcttttaaaggaatctgacataaattctgcgaggcagaaaccacctctgtaagctgatggtgtgaggtaccaaaaagataacacacctcatggaaggaggaggcactgcagggcctggcattacaaatgtcaaaaaagacttgtggtttcctgtagtttcagagggcgactgactcttgagacagaaaaagatgatacacaatttaacgccagaaaaaaataacgcacaatttaatggtgctcagaattcccagattcatcccagggtgtgatggtccccactggctttccagggtgggcttgggacgcggaaatcacataaaatagaatgtcaccccgcatgatccgggatcaaaggtgcacgtgtaggacagagctacaggacgtgtgcgtcaggctcacatctgcatggacgtggtccccgcagtgatttcctcatcgccctctgccatctcacagacttgggagagcccatcgctgggcagtcagaacctggaagtcatcccaggtggtctgcgttatatatcccatctcccttccactgcaccactggcctatctttagacgcacttttcaggtactaaaacaataaaataaacacattggcacacactgattcttctagccctgtatatttgccttgaaggccaaactcttgcctctgtcttgggtggctagtctcaatcacgccattctttacacccaatctaagctggcttccttttctccagattccagcacgctgttatttgccagaaattacactcagtgcttggaaagggtagtggttcccttccgacacaagctggcaggctctcggagaagcagtgtggtcctggcgtgacctcggctaaactccagactccttcgctcggggctgtgtgtcccctggtgaggaacttagcctctctgggccagtacaggagcacggcgtgtttagcagttatgctgcagaaattcactgattcagacacataaagccggacgcgtaggcctgtacagaacgacagttaactgcaacatgctaatggctgtggagtggttactggaatgatgctgtgttccttctgtgctgttcatcatttgtttcaatttttaaagcaaaaatgaaaaccaagatgattaaatatattaatatataaaaattatattataaataacaatgaataaataataaatgatataatacacaaatctatacaaatacataatacaaatattaatatataattgtatgtatttaacatatagttaaatatattaatatataattaaatataataatatatttaatcatcttgggttttattatatatatacacacagacacacacacatatataaacctaaaagccaatataaatctacattttcaccagtgactcccacaaaggaataaatgtcagaagcttctttaacaggattaaaacagtcacgtaagtgaaagtataagagaatgatgtttttgtttccactggaacataatcacagttatctctggttttttcttaaacggaaactaactgcttggttttcaaacaactgcacttttaagtgggcaaatgtgtctgagaaaggaaaagaacagggtcagaagaaggaaagaaaccaatcaggttagacagagcgtagatgctccagttcttgactctagcagtatcttcatgctacaccatcaaatgattttatccgcctagtactcttttactgatccccttttaccagtctagtaagcaacaggcttcgtgcaaacatgatttcaagtgtagtgttagcgtgttagccgctcagtcgtgtccaactctttgcgacaccatggactggagcctgccaggctcctctgtccatggcgttctctaggcaaggatactggagtgtgtggccattcccttctccaggggatcttccagacccagggatggaacttgggtctcctgttttgcaggccgattctccactgtctgagccaccagggcagcctaatgattgcaaacacagtaaaatgcaaatgctctatgtcctataaaaatcaagtataaaggaaaactaggatttaagtgaatatcctagtttaagcttaatctgatttccttcgatcctgtccaaatttccctggggtcctaagatcaccctcatgaattatgcaatgtgttaaatccctaagtaagtatactggagtgtgtagtatagcttcccaggtggcgctagcggtaaagaacccacctgctgatgaaggagacataagaggcggaagatcccttggcggaggaagcgacaaccccctccagtagtcttgcctgggaaatcccatggacggaggagcctggtgggcaacagtccgtggggtcgcaaagagtcggacatgactgaagtgacttagcacgtctgtactacagtttggaaggtatatcaagatgttctggggatgcgatttctgacaacacagtaaacagaacaaagtgtgaccccgaaatggtactgggaagatgccattggtaacagggaactggtgctatgtcagattacctggttgtgcaaatagaactattcacaagtcacacactgccctaaacacacagaaaacgtctctgccaaaaaaatgctgaaacgccctgcagtttcaagtatgacatttacatggttttatagaaataccacagattaaaaataaaagacagtgtcttttggtaggagagaaaccttgttcctccaaagagtatatgagaaaactaaagtgaactttgagatatctttctcaatttggttcctccattatacagagatttacttattcaaataagtagagaagagacagccccttacaaagaaaaggatgcagggtctggaagtgcgagtagaaaagttaatgtgtcttaaacgggtgactctccaacttgactggggactttatactgtgctctcctgctggcaaaggtcagtacagtcaaagctatggctctttagtagttacgtacagatgtgagagtcagaccataaagaacactgagtgtctgagaattgatgcttttgaactgcggtgttggagaagactcctgagactcacttggacttaaaggagatcaaaccagtcaatgctaaaccctgcatattcattggaaggactgatgcagaagctgaaactccaatcctttggccacctgatgtgaagaactgactcactggaaaagaccctgatgctgggaaagatggaaggtgggaggaggggacgacagaggatgagatggttggatggcgtcaccaactcaatggacatgagtttgagcaagctctgggagttggtgatggacagggaggcctggcgtgctgcagtccatggggtcgcaaagagtcggatataactgatctgatcaacaacaaatctcctactgaaggaagtctcctgggggctccccccctccccataaagaataaaaatctctatctgggaccttaagaactgcagtccaggagacacagatacaggtaaaaccaaaggaagcgttcggggaaaagaaagaaccaggggctcgtaaagacagaattcacaagggtgttacagttgcccagtgagaattatgagtgactcggatgcaagtcacaggttatttgtcctcacagaaccatgagttattttagggttcccaggggcttttttttgagacttaaatgtgaaacagtgacagcaaaagggttcagaacagcgtgattaattaaggagacatttgatgcacccactgacaccccggaaaagcccctgtacacaggccaagcactcgacaagtgtgaatacgctcagctgacttctggggtcttaacctgttcgattcgattagcgcttggtagatacgttcttaaagggccagagactaaatactcttcattccatgagccgcagagagctacaaccgtgcaatacaggcatcagcgggtttagctgtgttccaaggaaacttaattacgtataatggaactgagagaattttcacgcgttataacttgtcattcttcttggcttcatttttcaaaaaacacttgctttatttatttttaaattatttatttggctgtgactagttgcggtatgcgaactcttagcagcatgtgggatctagtcccctcagcaggactgaaccgagggcccctgcattgggagactggagtcttagccgctggaccaccagggaagtccctggtttctacatttttaagtggtccccccccccgcaaaaaaaatcaaaaaatggtggttcagtggttaagactccatgctcccaaggcaggggtcccaggttcaatccctggtcaagtaaactagatcctacgtacctcaagtaatgattccctctgctgaaactgagacccagagcagctaaatgaatgaatgaataaataagtaagtttaagttcagtgcagtcgctcagttgtgtctgactctttgcgaccccatgggctgcagcacgccaggcctccctgtccatcacccactcccagagtttacccaaacccatgtccattgagtcagtgatgccatccaaccatctcatcctctgtcatctccttctcctcccgccctcaatctttcccagcatcagggtcttttccaatgagtcagttcttcgcatcaggtggccaaggtattggactttcagcttcaccgtcagtcctttcaatgaacactcagtactgatttcctttaggaaggactggttggatctccttgctgttcaagagaccctcaagagtcttctccaacaccatagttcaaaagcatccgttctttggtgctcagctttctttatagtccagttctcacatccatacatgactactggaaaaaccacagccttgactagatggacctttgctggcaaagtaatgtctctgctttttaatatgctatctaggtaagtaagtactgggttggcaaaaaaaaaaagaaaatgtagaaaccatccttagcatatttaatgtacaaaagtagacaaagggttaaatttggcctgcaggctagctctgctaaagctgtgttagataagatgaggcatggttagattccattgctatcacagcttgcaaagattactgaaaaaccaacctcggaaagcattatgacaattcgtcaaaacattcaacacacagttgccatatgattcagcaattccacttttaggtataaaatcgaaagaacagaaagcaggaacctgaacagatacttgcaccccaatgttctaagtgacaacattcacaattgccaaataacagaaacaacccacatgtccactgatggatgaatggattaaaaaagtgttgtttattcatacagtgcaatgctattcagcttaaaaacagaatgaaactctgagacatcctacaacatgggcaaacctcgaagacgtcgtttagtcgctaagttgtgtccgactctgcgagacgccatggactgtatcgtaaaggaaatcagtccagaatacacattggaaggaccgatgatgaagctgaaactccaatactttggccacctgatgcgaagaactgacttaatggaaaagaccctgacgttggcaaagactgaaggcaggaggagaaggggacaacagaggatgagatggttggatggcatcacaaactcccggagttggtgatggacagggaggcctggtgtgcggcagtccatggggtcgcagagagctggacacggctgagcgactgaactgaagtaatgcactgcagcccaccaggctccaatgtccacggaattttccagtcaagagtactgggagtggggtgtcattaccttgtccatctgaagacactatgctaattgaaatacgtgagacacaaaaggagaaataatgtatgatcccacttatgggaagtatctagagtagttaaaaatagagatagaaagtagaatggtcttgccaggagaggggcgaatggagagcagagttaagtttaagagtttaaatctggaaaggaggaaaagttctgcaggtggatggtgaatgtacttaatggtacacagcacaggaatactgtgtacttacaagtggttgaaaaggggaacttttatgtaatgtatatacttttccataacaaaagagatctcaaaggaaatgaaccttcagtgcaaatgtacttcttccaaagacccagcggctagttacaaacttagatcaaaatgtttgtgacagctaaaatgtgcattcgccagtctactctgaagaaaagtcaagaatatctgctttttccataagccagagccaaatccttcactgtgaaaagaagctgtcctcggttatcttccaagtcacagatgacattttaagatacttgccactgcagcggaggcattccctagaaaacagtctcaaaatctgggtaatttctggaatttctgaacgttggtcaaacctagaaattcttaagccagcttctgtccttggagaatttatcttcatggaagcagaacgacacattttggctaattgaatttgtattcatctttgtggttaggaaatgcgattgatgttattgctaaatcaaggtttgtcataaaagtcattcaaaaaaacaaacatacgatgctgttacgtgattctcaggaccatacttgtggttgttagtaagaactctagaatccaggaaaaaaacagcagctaatcaatctaggtgggccgttccatcctacctaagatgaaacctgaaaagtgagggggaaaattgggggtgggggtggcagatctaagcataattctgggtggaagctatttcccttatttaaaagaaaagagttaataagaataaaatgagattaatgcatgttctgtcaaaaactctcccgatggctggttcactaaagtctgatctcctgcaaacttggggcttcccaggtggctcagtggtaaagatctgcctgccaatgcaggagacatgggttcgatccctgggttgggaagatccctggagaagggaaaggctacccagtccagtattctgacctggagaattgcatggactgtatggtccatggggttgcaagagtcggacatgactcagtgactttcacttttttgmtgaatgaaaatacacaaggggttatcaaatgactttggaaaactttcaggcaccttaggtttccaaaaggaatttttgttttttaaattggcctcacctctyggcttgcaggatcttagctccccgagcaggtatggaactcagggcccagggagtgagcacgtgtagtcccaaccactggactgccagagaatccccaaaggcctgtttctgtcatttggttttctcccagtgcgtgcatgctaaggcgcttcagttgtgtccgactctgtgcgaccccatgaactgtaagcccaccaggcttctccgtccctgggattctccaggcaagaatactggacttgggttgccgtgccttcctccaggggatcttcctgacccagggatcgaacccacatctcttccatctcctgcactgccaggcaggttcttttaccactagcgccacctgggaagcctggctttctccctcactcgactgcaaatcgaccaccttttacgacaggactatgctgcgtccagccgccctgtgaacacacgcagcgtccctgcccgcttcaaatcccagatccacatctcaggtcccaggcctcgccgctgcacgctcagcccaggactggcccccatgacgcgtgcacgtagcggaccctgaggcatctcgaacatggcgtgtccacgtgcgtccagcctgccagccccaaccacccacaccaacacacccccccactccagcccaccttgagagttaaaaatgacccctgcctcccacccagggggacacaggccagacccgtagttcctccaaccaggatatccacacatccgctggggctcctggcccaagactgtccgagtctgcgcttttccttctcagatccgctgggctgtgctagtgggaaataagactgccttcatcctgggacccactaggaccactccagttctgacccgcagcagcagccagccaccagtcacagcggtgggaaaagtctgcaggctctcaacttgaaatgcagcaccgtgaccatcgtatcccgcaaggagccgtcgggtgggccccgcacacagctctcaacctcatctcctggtcggtctcgggcccccaccatgctggcctggcctcctttttgtccctggggtttccagagctcagcgtcacaggctgccgctttggaggtgctgttccctttctctggaactatcttttccccgctcttcccagaaacatgcttcagacgacggctcagcagtttccccttctaaaagctcccatgtagaagcccttctggtccgaggccttcagctccacggccccactccttcttgaccacatgcgtcaccatcaaagcatcctgtgccatctgggtactgcctgtctctcaggtggcaggcaggctccggcaggacatggcctctggaccaccgtatcgctgagctcagagttctccctccagccacaaaactgagtttcgtgccagatgcgacaaacacaggtgatcaacagcctacgacatacgagcacgaccttaaaagtcacaatacaacacaaaagggacatcccgggcgtccagtggttaagacttcaacttccaaagcagggggtaatgggttcgatctctggtcagggacttaagatcccatatgcctttgccggggaggggcaaaaaacaagaacatgaagcagaaccaggatgggaacaaattcagtaaggactttaagaaatggtccacatatgaaaaatcaaaaagagaaattaaggacacaatcccctttatcgtcgcaacagaaagaataagatcccttggaataaagctacccaaagagagaaaagacttgcatgcataacactataagaccctgatagaagaaatcaaagatgacgcaaacatggagagatattccatgttcttggattggaagaatcaatactgtgaaagtagctatgctcccccaaaggaagctaccagatccagtgctatccctatcagtaaccaatggtattttccacaggaggagaacaaaaaatttcacaatgtgtatggaattcaaaagacctcgaataaccaaagcaatcttgagaaagaaaaagggagctagagaaatcaaccttccaaacttcagactctacagtctgaaaatagatagcttcggtcatcaagattgtatggtactggcacaaaaacaaaacgacagaatgcccagagataaaccaaagcacctatgggcaccctatctttgacaaaggagccaagaatacagaagggagaaaagacagcctcttcaagagttggtgctgggaaaactggacagctacatgggaaagaatgcaaccagaacacttcctaacaccaaacacaaaagtaaactcaaaatggattaaagacctaaatgtaagaccagaaactgtaaaactcttagaggaaaacataggcagaacactcgatgatataaatcatagcaagatcctctatgacgcaccctctagagtaatggaaataaaagcaaaaataaacaaatgggacctgatcaaacttaaaagcttttgtacagcaaagtaaacaatcaacaaggtgaaaagtgagccctcagaaggggagaaaatgatagcaaatgaaacaactgacaaaggattaatattcaaaatatacaagcagctcttgcggctcaataccagaaaaacaaacaaaccaatcaaaaagtgggcagaagacccacgtatctcaaaaaaagaaatacaggtggctaataaacgcatgaaaagatgcacaacacgactcatcattagagaaatgcaagtcaaaactacaatgagatacgaccggacgctggtcacaatggccaccatcaaaaaacctacaaacgataaatgctggaggcagcgtggggaaaaggggaccctctcgcactgctggtggaaatgcaaactgatacagtcactatggacaacggtgtggagaatccttaaaaacagaggaataaaactaccatctgacccaacaatcccaccactgggcatataacctgagaaaactggaatgaaagagacacacgtaccccagtgtccactgcagcactgtccacaaaagccaggacgtggacgcaacctagatgcccatcggcagatgaatggataaagaagctgtggtacatatacacaacggaatatcacccagctatgaaaaagaacacattcgagtcagttctaacgaggtggatgaacctggagcctattatacagagtgaagtgagaaagagacaaacactccgtattaacgcatgtatacggaatccagaaagatagggctgatgaacctatttgcagggcagcaacggaaacgcagatgcagagaacagacttgtcggcaccggggaaggggaaggagcgaaaatcggagagagtagcattgaaacatatacagtactgcatgtagaattaaaagcggcagtgggaatttgctgtatgacgcagggaggtcaaatccacctagatagggtgtgaggtgagaggtacgttcgacagggaaggcctcacacataacctgtggctcactcatgctggtgtcttgcaaaaaaccaacacaatattgcaaagtgattatcctccaattaaaaataattttttaaaaaagagagagaggggggaaaaaaaggcccatgttaaaaaaaacaaagaaaaaccaaatacgattacttaagaccaaactccagtgaagatggcactttcctccttcatgactcacgtgtggctttcaggtcgcgctgggcttttatactaagcgctaacgacaagcagaaagtgtgtgtggaagttgcctggaatcatatccttctaaatgcatagttgaacgagcagatggcgtgacacctccacatcacctacatacaagggtatatcctcggcattacgtgagacagacaatctggcaagtcctgcgcgaacacgcacgcacacacatatgtaattaccagcgtccgctcttcgctggccggagaaggcaatggcaccccactcttgcctggaaaatcccatggacggaggagcctggaaggctgcagtccatggggttgccgagggtcggacaggactgagcaacttcactttcatgcactggagaaggaaatggcaacccactccagtgttcttgcttggagaatcccagggaaggggggagcctggcaggctgccatctatggggtcacacagagtcggacacgactgaagcgacttagcagcagcagcagcagctcttcgctggcagggctaacagataaaaagatgaatggcaaaaggatctcgtaaaggattcagccactcccaggggctgaagctgctgccaccagccaggtacagatttccacgagcacagctcaggaaagggacgccagccagtccaggctgttgacaggtccttacccgtgctctgtatctcctctacagaagaaccttaaagcgggatgcccgacgcacagggatgggtgaacacagctggcaaggtagtgttcacaaccatccgcgcgtctttcctcccggtacctctggatatgcccgctcccaaagccggtccggctttgctcagctgcacctgctgctgtaatgacacgcgttcctgagatatttcccttgagaaaatatgagtgcatacagcagccgctgaccggatttttgctaaagcagagttgatgctctgcagacagggtaaatggcaatcattttaacagaggccgtggaatcacatgtgggaaagacaaccacgaagggcttccctgagggctcagctggtaaagaatctgcctgcaaggcaggagaccccggttcagtccctgggttgggaagatcccctggagaagggaaaagctacgcactcaagtattcctgggcttccctggtggctcagctggtaaagaatctgcccacaatgggagacctcggttcgatccccaggttgggaaaatcccctggagaagggcaaggctacccactccggtattctggcctggagaattccatggactgtatagtccatggggttgcaaagagttggacacgactgagcggctttcactttcaggataaccaagactgaaacgcaagaagaccttacggtccttgcccctcacgtcccccacctccttttttgtctgtggaaaaacgttagccaaagaataagtttaatcagaggagtgagaaaatgcagaaacataggaaaacagtcaaaggagactaactataaactattaacaatttagccactaagaattgatggcttcgaattgtggtacaggagaagggttttgacagtcccttgaacagacagcaaaataaaagcagtcaatcctaagagaaatcaaccctgaatattccttggaacgaatcgagctccaatattttggccacctgatgacaggagccgactcatttgaaaagaccctgatggtggcaaagattgaaggcggcaggagaaggggacgacagaggatgagatggttggatggcatcaccaacttgacggacatgagtttgagtgagctccgggagttggtgatggacagggaggcctggcgtgctgcagtccgtggggtcacaaagagtcagacgtgacttagcgactgaactgaactgagctgaatgtgcacaaatcaaagaactcattagtgcttccaaatgccaggagtttatgacagctaacagtgattacacacactttctggagacctgagaaagcccagagctaaatggctataactttataatatgaaattaatgcagcaaaatcaccacaatatcccagagaccgctagtattctgaagccccttcatgagatagtagtatctgaatgctgactctataacacagataaattaagaatatacacaactagcttcttgggctcccctggtggctcagatggttaaagaatctgcctgcagtgtgggagacctgggtttgatccctaggtctgtaagatcccagagaaaagggaatggcaacccactccagtattcttgcctggagaattccatggacagaggagcctggtggggtacagtccatggggtcagcaaagagtcagacacaatggagcaactaaccctttcactttttcacaactggttttttaacattagaaaggatacgcaatggaattggaactaaattcatattgaaatgagtacattctgcactttgttgaatgcattagctcatataaaatcatttatgacagtttttttaaaagcagctatctgtgaagtgagatatataaccatgaaatacgataaattttttaaaaaaggatacatcctgctgatccttttaatctaatccctcaccgtatctccacttctgccttcactggcgtaaatttatctccccaggaggtgttaaatcaaagccttctgggcctcaaaaggaatggttcgttggttgcatgcttgggctccagggtccgcttgtgggcatcgccatgaaagaacctacttcatcatgttaatctacgcctgtgttgccactgctgcaaatcatttcatcagcactatcttcgcagattccatatatgtgtgttgcgtgtgcttagtcactcagtcactttgcgaccccatggattagacagtccgtggaatgctccaggtcagaatactggagtggatagcctttccctcctccaggggatcttcccaacccagggatcgaacccaggtctcccgcattgcaggcagattctttaccagctaagccactagggaagccccaagaatcctggagtgggtagcctatcccttctccaggaggtcttccagttccaggaactgaaccggggtctcccacattgcaggcagattctttactagctgggccaccagggaagccccaagaatcctggagtgggtagcctatcccttcctcaggtcttccagacccaggaattgaactggggtctcccacattgcaggcagattttttaccagctgagctaccagggaagcccaagaatcctggaggggatagcctatcccttctccaggaggtcttccagacctaggaattgaaccggggtctcccgcattgcaggtggattctttactaagtgaaccacccgggaagccattaatatacaatatttgttttcctccttctgacttacttcaccctgcattttgacttgtcaccttgaaagtgtatgatttattataagattatgtctgttcaaatatacttataaaaggtttttcattttaaattatcagttgtgtttgacatagggatttgaatttatcctcatgatttaagtccctatgatgtaactaatccaactctaattcactcagtatatatagaatgactttgcaactactattcaaattaaagtactagccaatctaacatagcctccttgtaaaatatatttacgataaaaaaaagctgcaagggattaaccttaaaacatttttaaaaaagaacttaccttagtacccctctgatggtttctttcatttccgaagcccccccaaagtgcaagagctaagatcctcccatctgtctgcactgacctacctactgtggtagttctcactgggggcgatgttgccaagcactcagggtggggggacatccgacaacatgtagagacatgtttggttgcttcaactgttaggagagagttcctgtacactactgcatagaaacccaggatgctgttccacaccccgcaatgcagagaacagttcctaccatcaaaaactggtgtcactgttcagttgcccagtcgtgtccaagagccggaccgtaaacaaggcagagtgccaaagaactgatgccttcgaactgtggtgctggagaagactcctgagagtctgcccccaaaacccagctcagttcagttgctcagccgtgtccgactctgcaactccatggaccacagcacgccaggattccttgtccatcaacaactcccagagtctactcaaactcatgtccactgagtcggtaatgccatccaaccatctcatcctctgtcgtccccttcttctcctgccttcaatctttcccagcatcagtgtcttccagtgagtcagttcttcacatgaggtggccagaggattggagtttcagcttcagcatcagtcctttcaacaacacccaggactgatctcctttaggatggactgtgttggatctccttgcagtccaagggactctcaacagtcttctccaactccaaatgtcaaaagcactaaggttcggaaacccattttatccccttatgtatgcggggaatttatctcttcttcctggagccagttttcacagtctatgtaagttgtacctttttttccccaccctgttgttcctaaattgtccatgagacactttcatttttgtaagaactcagtcttgcttctgtctttccagcattcagactcttcacgtaatggacagctgctgggaataagtcgtcatgaactgacaatccagacacctttgacacctcttattattttttttttatatttacttttaktcttgcttatttgcttgcaccaagtctcagggcacatggggtcttttgttgcagctcacgagaccttgtgcagtggcacctgaattcagtggtggcatgtggtacctatgaacccgggtttccctgcattgggtgcagtgactctcagccactggaccaccagggagatcaacatacctctctttggaagctactatctcacaaagataactctcctccagtccccacaggttaattctcaacgggaaacttctaccttcttcccaaacttaacagacttaacaacactggagataacaccactcaacttgacgaattctaagtcagataatcccaccatcaacacaccatctttcactccaaacatttagagcatcagcttcatcgagatggggaaaacaaaaacaaaacagcaggccaaaagtgaaggatgctctgaagcttctagaaggcarcaaagctaaccttctgctccagggagaagacattctgtatgtggtcctgttttgaccagagaacccaggcctatgatcagggcaaggctttgtggaaaggacggagggggcaggcgagaaacggaagaccacggtgatcattcggacatcaacaggctaagaattcacaaggcatctatttcaaatgccgcagaccagccgtcatgaacgctgtttgatcagtgtctaccacgaaaagctgtacctcccagacatcacaaatccagcctattcagtcccaggtcggtcaaataataataataacaaaaaacacggtgtcttgtaggtgtctgtgctgtctccacccccacatcgcattattcctacaggattttaaaggctcaatgagtcaaaatggggtcacacagatgaagtccacatttaagcactcatgataaatgggtctgctcctccgtccacaccagggaggtaaaagtccgccctaaatgcaaggaagtccatcatcacacatgggggcctcacacagctaggtctctctgcatttatctcttcttaatttctacagggcacaagcctccattcaacaatacgattttcacggaacggaaaattcacggtgtcaggtcaagaggagcgttgagaaccgatacgctggataaaaataattataatgatctaccaagcgccctggaaagcaaaacagcatgacaggcccagtaatacataaacgtgttccatctgcatacattgtaataacacggccactggaagaaaatccagtgtgaactttccttactgcctcagcgtctgtgatttaaacaaaatcaggggcttcttattaatcagttcaattctcctctgaaccaccatccactccattcagcctgcacaaccaaaaatttctcattagcttaaaagagaccgtgctcagggaaaatcattgtgtgtgctaatttctaccaaaaaaaaaaaaaaaaaaccaggagaaatcaatttttccttacaaagtttctaactggcaaaggtccactcaagagtgctgtggttttctgctccccccaccaagtactattatattaatataaagggacataatagtttacatatttgttgaaaacacagaaagaagttaaatgaactttccttttctatttacatggctgaaaaattatgaacaaataatccaaacacaccatattcacatttaaaaatattcaatgaatactcaacatttctactctatttcttttcactgggtggggatatgtgtatgattgttagaatctgctctctagtaagcaccccactccagtactcttgcctggaaaatcccatggacggaggagcctgctaggctgtagtccatgaggtcgcatagagtcggacacgactgagcgacttcactttcacttttcacgcactggagaaggaaatggcaacccactgcagtgttcttgcctggagaatcccagggacggcggagcctggtgggctgccctctctggggtcgcacagagtcagacacgactgaagtgactcagcagcagcagcagcagcaggagtattcttgcctgagaaatcacagacagaggagcctggtgggctacaatccatggggtcacaaaaaagagtcagacacgactaaataacaacacccaatgtgtcatacccaacacatacttttatccaccaggatgtacgcgttctgtctaatttgttcactgttagattctctgcatctagaagaattcctgagacaggtggcaagtgctctagaaacatttatttactggttaggtgaacgcgttcagtctgagagatggtgtgaacgcaaatgacccaggagcatacaagttcatcaagaacttcatgtgcaaatattctgcaaagtgctacgtggtgtggaaggaccgtagagacagacgcctaagccccttcctgtacaagaagcctgtgctacaaaccacacaggctttgcataaaaagataactcccaacaaacagcacattagactaagtctgaccgaggtaggaggaagacaacaaaggcatgctctttcatctttattacttcatgcaaccactataaactccccggagatcatattcccacttggtgcacaaagaaagaggttcctggtcacctggctgaaactcagagtaagtggtgggcaagaaaggaaacacagatgagcagaagatggaacccagaagctctctgcaggagccgttgctgtaccatatacccccatcctcgtcccacagtctagawttctgcktkraacacgtgattgttttagcaacaccgtgttcccactagagtgaggacaaggtcgaaggtagaatctgctagactaccattatcagactaccattatccaattccatccctcaccctttgagaacaggagacctggattctgtgtgggcacatggtcacacccctctcaaaaggacaagttttccccgtaacccagttctagccaatggctacaagcaaaagtcaaatatgaaatctctagtctctgctcttaaaggcaatgaaatccccctcctcactcctactcagggatgtgattgtgagggcaggagctggggcagccattccagacacagaggaggacagaactttggggctcctactgtgaggcagtgctttatggaacgagaggtccatcaccccacttgcaaagctacagaaagttacatctcagctgacttaagtgactcaccccatcactgccaaattaccttgcagaaaggtcagaagctgcttcctggcctcttataacaacagagacattcatcaacagggaagaggaagtgaagaaattacaaactacagctactaatgatttcctggtcagaaacctaagacttcacagtgtgttttggaaccagtgttcattcatgggcaccctagggtgttttaccatataaacaaagcctgaagaaaatccctttcatcagaggaggcatcagttagaggcagccctgagtttatgactttctggctctgaacctgtatgagtttcaaagcgtgtgcacatggggacccctgaacttatagggtgccaggctcctctgtccgtgggattctccaagaatgcaggaatactggagcaggttgccagatccttttccaaggatcttcccgagccagggatcgaactggcaactcctgcaatggcaggtgggttctttatcactagcatcacctgggaagcccaattcaacctctgagacctttttttaagtgcacagcgggaagaaccaaccacctttatctcacgggggatgccaaggggtcagaaaaacagcgtagaccgaggacctcgttcccttccacgtcagcaccgccttttgtggccaggaacccatacatccggcagaggtaaggggaggtggccacgcatgaacacggctcacagcgaggcaaacaaatcctcatgctgactcacctgggtatacagaacactttaggaaggccgacagtgaaagcagggcccgtgttcagactcggatgtcacaaccaacccaccgcctgtgatctcaagcactgcttctttcctttgtggtgtgagagctctgatgggagatgtggatgggccaacggaaatgcgacgtggatgggccaacggaaatgcgacgtggatgggccaacggaaatgcgatgtggatgggccaacggaaatgcaaggtgagcttacatctaattaaagaatgtttaaacgcaaactaattttcacaacatatttaaacttaacccagcatatcccaaatagcatcatttcaactgtagttcacactggtttttaaaaaaattattgagatcttcgacattccttttacctacaaaatctctaaaattcccaaagtctatgtattttccacttgcgccacatcttaattaggaaacggaattttcaaaggaaatagaccacggctatcagagttcagaagatgtattccgtacactgaaaacacaggtacacacacccatgtgattcgcaacatacaacttatggagcagctgaaccaaggatcgaaatttaaatcaataaggaaataaaatctctaaatctgatcctccgtaaccctagctatatttgagggtgctccttgggtctacaaggcttgtggcccctaaactgctaagaagcctgcaggagtgaaagtttgatgattgtagtctgcaggatggaaggacctggggacattccaggccacacgattcctctgtaatcggtccacatgcaaattcttaaattatgcacccacagcatttttttttcccctcgacaaatttcaaatagattaaaaagaaaaccggacaggccacccaacttttaaattttctgaaaatagtgtaacttcctgctagaaaattatttttcataatgcacattttcatatttttacatagacactggaatttgggggctcagactggactgaccaaggcaacgagcccaaagtaagaagtgctgtaggtttaaattccacgctggattttgcagacttagtacagaaatggaaagaacataagatatgtcttggatgattttttaaatatgagtaacttgtcaacatgacactattttgggtagtgagttgattacattaatacatatgttgcaaataatgggcttccctggtggctcaaacggttagatttgcctgcagtgcaagagacctgggtttgatccctggtccggaaagatcccctggaaaagggaatggcaacccactccagtattcttgcctggacaatctcatggacagagaagtctggcaggctacagtccacagtgtcccaaagagttgggacatgactgagagactgttttttttcattgttgcaaataatattaatcgagcatatattttggagaaggaaatagcaacccactccagtattcttgcctggagaattctttggatggaggagcctggtgggctgctgtccatggggttgcacagagtcagacatgactgaatcgacttagcacgcgtgcatgcattggacaaggaaatggcaacccactccagtattcttgcctggagaatcccagggacagagaagcctggtggactgccgtctatggggttgcacagagtcacacaagactgaagtgacatagcagcagcagcatatatttagatatatattgaaaaaataatttatgatttctttttacttctctactgtggctactagaaaaccttaaattaaatttatggtttgcaatctacttctatttggcaagtgcaaaaacccagacagtttccatggtgcctgcaggctctaaaatgtgttgctaatagtcagctagatgaaaccatagcatttttccaaggcttcttttgtgtccactgcaaaattttaattagatgcccatcgcatcatttttttattccgtgaccaattcaagacagattaataatcacaaattgttctaaatgcagaccacctttctctagtaaaaaaaaaaaaaaaacaattttcagtttgaggattttttaatatagggacttatcacaagttctgtcaagacgcctgaaagagttgtgggtctcaaaaaagccacggtcattggcaggttgctcgctttgtgggcaaacaccgacctctcacctgggtgtgtactgttacaccttccagtttcagagccacctggcccagcagacagcaaggtgcgctgtgaagtccagggctcactgcagagcactgggtgtggaccgagactggctgagcctgtctcatctataacctggggggtgacgtcaccagagctgcacccctcttcctagcaggttgccatgggagacccagggtgatgatgctacaatggactctgtaccacagtcaacagtacagctcaaatttcagtgcgaccgtgtttaagatgggcttccctgatggctcagtgggtaaagaatccgcctgcaaacgcaggagacacaggagattcgggttcaattccctggatcgggaccaggaaatggcagcccactccagtattcttgcctggagaatcccatgggcagaggagcctggcgggctacaatccaaagggttgcaaagagtcggacacgactgagcttgcacacattctgtttaagatagggtgctgctgaacagatattttagccaggactaaggacagtttttcttgccgttattgttatattaaacctcctgttagacaaggcttcctgccccttacactgtcggctatcacttttaaagtttaattaagcctcgcctactcaaagcgctggagaacccacagctagagatgcttaggctcccagcacagggagctgtgtgcatctcatcaatgatgacaaactgaacattcgaaggcctccttttgcaagaggccctttattgtcactttgaggtctactttgaactaacaataaaaaaaaaatacaccattcagagatggattggttgagttgcctagccactgagttgttttctagccactgagtctcctgagtcgtgttccactcttttgcaacctcaaaagactgtagcccaccagactcctctgtccacgggattctccaggcaagaacactagagggggtttccatttctttctccaggggtatcttcctgacccagagatcgaagccgagtctcctgcattggcagccgaatcttctgcattggcaggccaatcctttactgctgagccaccaggcaagcccaggttggtgttaaggagaaggagtttattttgcaacaatggtacgacagtataggagagctttctacttttactacttgggcagagggaatatggcttccgtttgcccacagttggaaccggtaccctgcacctatctgtgttaaccacacgccctgggaaacggagttcaaactgagcatctctcaccttgagccaccgtggggaaaggaacctgggagtgagtcctctccccattcctcatttctgtgtgtaatcgcagagtgtagaaacaaggaaactcctagtgtgtagcagtaacatactgagaggaaacaggaccctgagacctgagaagatgcatgttactatagaaataaggtcatttttccacgtattacctcatcgattgaaatctggcaggaaatgattcatggctagactctcacctattgaacataatctagagcaaaaacattcacacatcattgagaagcgaaagccgttaagctacaaggcgacaagaaccgtatcccaaaatcattacatttgtttttaataatcaatgaatttttttttgttttttgcgggcacatcaagaggcatgcaggattttatagtaccctgaccagggatcaaaccagcaacccctgccgtggaagtgccgaatcttaaccacctgaccgtcaggggagtccttctactgatcattgctgtatcatctttttcctcgcctttcattcatttcctttccaggtttggaaatggaaaaaaaaaaaaaaaaagaaatacttaaacactctaaaaggaggaactgaaatagatctttgcaaagctttccaaatgtgtgatgttacacaacagcgcaaggagagagaagcaacagcaaacgcagagacgcctcactgatgacttcagagcaacacagaaaacaaccaatcccctccttgtccctagcatgccacgaaacggcaccgtgcttcaaagacacggcagagacattcgatccattcatttgcacagcaaaatatatgtcacccgtacacactcagtcacacctagacacacacccttgggaaacaaacttgggctaatgacaagtagaagcaaagtgcaagccggcctattgataattataaatattttaagatcttttgcaggctaacctaagaatctgcgcttttcctacagatgtcattctgtaaaaacatcgtcttctgataaacacactgggaatcgtgagatgagctgaactcacgtagagcagtgatattcaaaggcatcaattctcatccatgagcagtcacagcgcacatccccccctccccccacaacggctttaaggaaaggggtcaaagagttaatgagcaaagaaggtattttggtataatctcagtaataagttcatatgctgacccgccttcctgctttctggactcaactcctgtttaagacgcagccccaatatacagactcacagtctggctccagtcgggagactcaacttcccggcaaacgccagaaacggcccgtgtagctgaaactctgacagtgcagtctttattaaaacagctggcattcacgtggaacccagagagcgtaaccccaaacagaacccgagccattgcttgcaaagcaacttacgtccaagtcacagaaaaaaaaaaaaaaaaaaatgcaaaagactggcgaacaggatgaaatagtccacctcattttttttttaaatcaaagacatgcaaaaacaaaacaaaaaaagtgcccacgttcccgtctcagttgaccacaatgtcctgagctgtctgagagacggggccccacccccaccccagcccaccccaccctgggctccctggcaacagggaacaaccagcacaaactttcaaggaaagcaatttggtaatcggtctcaagagccctacagctccattcccagccatctctcctaaggcaaaaaaaaaaaattggaagcaatctgaagctccggcaacaaacgttaaatcaattacggcacacaaactcgacgggatggatgcctgaaaaatgttttcaatgaacgtcggcaatgatcacaaggctcactccgtaaggttaagttgggagagccgggtcacaaagctgaaccttcctgacgtttcatgcacaggaatacaggcagctgggaggagatgtaccaacagtctgatgggggaactggcgtgggtgggctcagaggtgtttattttgtttccagattccattctttgctttttttcccaaattgctcattctatgcagcacattgcttttagaaatcagaaaagagggactgaaaatcctggccagcaaagagtaatgactaaattcctcatctgccaaggatgaagaagatgaattaagacagcgatgtacctgttcacctctttcccaagaacgtcaacgatggttttcctcaagaggcgcggtgtgccagctcggggcgggggcggggcggagattccttctcctcgggggttcacagtgatcagcctcctctgagtcaccttgtgaaggtttatgatgccagagtctataaacacaacattaaatatttatggttgatatttgctgcttctaaaaataacgtggaggagaaggtctcttaacaaggcctcctgttgaccttctcgatgaaatcaccaaggctgctgattctgtaaacacgatgacagccacccacaggccctggaaatccgtaggccaatgccgccttcttaatccagtggacccccccagccccggggggccctttccacctgagccaggtacccttggccttcataccccagtgttgatggggccccaaacctgcttatgcgatggggcatcttactccatcaatgaaatgtaacgaaatattacataagcttatcagttaggaagacagtctttttttccccctaccaaaactactttcgatgctttgttacagactaacagacacgtcatcagacaaaaatgtaccatgaggggcttccggaaggtccggtggttaagactcgagtgagtgtccaccgcaggggatgtgggtttgacctcctgtcggggaaactcagatcacacatgctgtgtgcatggtacagagtgcagcccaaaattaaaaaaaaaaaaaaaaagttatcatgaaaacgctggcggggtggacaagagtgaagagatctcagagaaggcagtgaacatgcagatggattccgtgcggaggtggtttccttcgggaaccactgcgcttcacgggactcaaccctggaaacggcagagggtgcatcatgggtacagtttattacggacagacgaccccaaactggaattactggacccgtattccaagacaaggctgaattaccttccatcaagcaatgagtgaagaaacctgcgtttaaatttgctgctgtttagatgctgtcgtatcccaccctttcgcaaccctgtggactataaagcccgccaggctccctctgttcacgggattttgcaggcaagatcacagcagcggggggctgccctggcgatccagtggttcagactccgtgcttcccactcagggggcacgggttcgatccttggcgggggaaataagatcccacgagcctcatggtgcagtccccaaaagttaaaaaaaagaaatactactactggagtgggttgccatttccttctccaggggatcttcccgaccccgggatcgaaccagcatctcttgccttcgcaggctgtttctttatcgctgagccactagggaagcctagcattcaaacagcttaagcttaagtctccaggaggtgacgcttaccttgctccccctatagcagtaagcatgggctttgaaagaagtgtgggctttgaaagaagtgtggacacacaagagaggggaaagtggtaactcgacactgaagggactctgcaaacgcaaaccctgagcagagggcaagctctgcaagctcatcagcattaggtcacgttgagagtcccggccctggaggcaacgtgatcacagggcccgtctctgcggtctatgtcccccaaactcataagcccagcctcatcacggggcacacacgtgccaagcccaaagtgagagacgttctacgaaagtcgcggccggtatccctcaaaacagagccaccaccaccaaacaaagtccgagacgctgccctggcccaaaggaaagatggagacaggacaactgagttcagtgtgatggggacgggaattctgagacaaaaaaggacatggatcgagaaaaggttaaacgtgaaaaataatcagatatacagtggacaaaatttttttaaatcatgtatacgtgtttcatttctttttttctaattgatttattttaattggaggctaattactttacaatgctgtagtggtttttgccatacttgacatgaatccaccacgggtgtacatgtgtccccatcctgaaccccc

(19) United States
(12) Patent Application Publication
(10) Pub. No.: US 2010/0107265 A1
(43) Pub. Date: Apr. 29, 2010

(54) Double-muscling in mammals

(76) Inventors: Michel Georges, Villers-aux-Tours (BE); Dimitri Pirottin, Sprimont (BE); Luc Grobet, Esneux (BE)

Correspondence Address: MCHALE & SLAVIN, P.A., 2855 PGA BLVD, PALM BEACH GARDENS, FL, 33410, US

(21) Appl. No.: 12/322,075

(22) Filed: Jan. 28, 2009

Related U.S. Application Data
(63) Continuation of application No. 11/416,780, filed on May 2, 2006, now abandoned.
Application No. 11/416,780 is a continuation-in-part of application No. 10/251,115, filed on Sep. 20, 2002, now abandoned.
Application No. 10/251,115 is a continuation of application No. 09/007,761, filed on Jan. 15, 1998, now abandoned.
Application No. 09/007,761 is a continuation-in-part of application No. 08/891,789, filed on Jul. 14, 1997, now Pat. No. 6,103,466.

(51) Int. Cl. A01K 67/027; A01K 67/033; A01K 67/00; C12N 5/071

(52) U.S. Cl. 800/15; 800/13; 800/24; 435/325

(57) ABSTRACT

The invention relates to a gene (cDNA) encoding a bovine myostatin protein. The nucleic acid coding sequence is identified as SEQ ID NO:1 and the protein sequence is identified as SEQ ID NO:2. A mutant gene (SEQ ID NO:3) in which the coding sequence lacks an 11-base pair consecutive sequence (SEQ ID NO:11) of the sequence encoding bovine protein having myostatin has been sequenced. It has been shown that cattle of the Belgian Blue breed homozygous for the mutant gene lacking myostatin activity are double-muscled. A method for determining the presence of muscular hyperplasia in a mammal is described. The method includes obtaining a sample of material containing DNA from the mammal and ascertaining whether a sequence of the DNA encoding (a) a protein having the biological activity of myostatin, is present and whether a sequence of the DNA encoding (b) an allelic protein lacking the activity of (a), is present. The absence of (a) and the presence of (b) indicates the presence of muscular hyperplasia in the mammal. The invention provides a transgenic non-human male mammal exhibiting muscular hypertrophy, in particular, a transgenic bovine. Methods for preparing these transgenic animals is also described.

### CROSS REFERENCE TO RELATED APPLICATIONS

This application is a continuation of application Ser. No. 11/416,780, filed on May 2, 2006, which is a continuation-in-part of application Ser. No. 10/251,115, filed on Sep. 20, 2002, now abandoned, which is a continuation of application Ser. No. 09/007,761, filed on Jan. 15, 1998, now abandoned, which is a continuation-in-part of application Ser. No. 08/891,789, filed on Jul. 14, 1997, now U.S. Pat. No. 6,103,466, the contents of which are each herein incorporated by reference.

### FIELD OF THE INVENTION

This invention relates generally to factors affecting muscle development in mammals, especially livestock. In particular, this invention relates to the cloning of the myostatin gene, a member of the TGF-β superfamily, its involvement in muscular hyperplasia in livestock, and a method for determining myostatin genotypes. This invention most particularly relates to the production of transgenic non-human male mammals exhibiting muscular hypertrophy, such as transgenic bovine.

### BACKGROUND OF THE INVENTION

The TGF-β superfamily consists of a group of multifunctional polypeptides which control a wide range of differentiation processes in many mammalian cell types. GDF-8 is a member of the TGF-β superfamily. All members of this superfamily share a common structure including a short peptide signal for secretion and an N-terminal peptide fragment that is separated from the bioactive carboxy-terminal fragment by proteolytic cleavage at a highly conserved proteolytic cleavage site. The bioactive carboxy-terminal domain is characterized by cysteine residues at highly conserved positions which are involved in intra- and intermolecular disulfide bridges. The functional molecules are covalently linked (via a S—S bond) dimers of the carboxy-terminal domain (U.S. Pat. No. 5,827,733).

Recently, it was reported that mice deficient in the gene encoding for GDF-8 were characterized by a generalized muscular hyperplasia (McPherron et al. Nature 387:83-90 1997). The GDF-8 deficient mice were produced by gene targeting using homologous recombination in embryonic stem cells, a method referred to as “gene knock-out”. The murine generalized muscular hyperplasia appeared to be very similar in its expression to the muscular hyperplasia characterizing “double-muscled” cattle. This observation raised the intriguing possibility that the bovine gene encoding for GDF-8(i.e. the bovine evolutionary homologue of the mouse GDF-8 gene) is involved in the bovine double-muscling phenotype. It also raised the possibility that the human gene coding for GDF-8 (i.e. the human evolutionary homologue of the mouse GDF-8 gene) is involved in regulating muscular development in humans, specifically skeletal muscle genesis. Isolation of the human GDF-8 gene may have therapeutic uses/applications in the treatment of musculodegenerative diseases through upgrading or downgrading the expression of GDF-8.

The occurrence of animals characterized by a distinct generalized muscular hypertrophy, commonly known as “double-muscled” animals, has been reported in several cattle breeds throughout the world. The first documented description of double-muscled cattle dates back as early as 1807 (Culley, G Observations on Livestock, 4th edition, London, G. Woodfall, 1807). One of the breeds in which this characteristic has been most throughly analyzed is the Belgian Blue Cattle Breed (“Belgian Blue Breed”). This is one of the only breeds where the double-muscled trait has been systematically selected for, and where the double-muscled phenotype is virtually fixed. A comparison of double-muscled and conventional animals within the Belgian Blue Breed, showed an increase in muscle mass by 20% on average, while all other organs are reduced in size (Hanset, R. In Breeding for Disease Resistance in Farm Animals, Owen, Axford, editor, C.A.B. International, pages 467-478 1991). The muscular hypertrophy was shown to be an histological hyperplasia affecting primarily superficial muscles, accompanied by a 50% reduction in total lipid content and a reduction in connective tissue fraction as measured by hydroxyproline content (Hanset et al. In Current Topics in Veterinary Medicine and Animal Science, volume 16:341-349 Eds. King and Mènissier 1982). Double-muscled animals were shown to have a reduced feed intake with improved feed conversion ratio (Hanset et al. Gènèt. Sèl. Evol. 19:225-248 1987). An important economic benefit of double-muscled animals, in contrast to conventional animals, is the substantial increase in selling price and net income for the farmer (Hanset et al. 1987).

One of the most through series of studies on double-muscling is that of Hanset and colleagues in the Belgian Blue Breed. Objective criteria of muscular development, such as dressing-out percentage, lean and fat percentage, plasma and red cell creatine and creatinine concentrations, were measured on nearly 150 randomly selected animals raised in standardized conditions. These studies clearly revealed abnormal, bimodal distributions of the double-muscled phenotype and objectively confirmed the visual classification traditionally performed by breeders on double-muscled and conventional animals. The phenotypic distribution was resolved using a maximum likelihood procedure into two component normal populations with a common variance which revealed mean differences of three to four standard deviations depending on the trait. This suggested the presence of an allele having a major effect on muscular development with a population frequency close to 50% (Hanset and Michaux Gènèt. Sèl. Evol. 17:369-386 1985). The most convincing evidence in favor of such an allele, however, came from experimental crosses involving double-muscled Belgian Blue sires and Holstein Friesian dairy cows (the latter animals having very poor muscular development). While F1 offspring showed a phenotypic distribution very similar to that of Holstein Friesian dams, backcrossing these F1′s to double-muscled sires produced a bimodal BC generation, clearly pointing towards the Mendelian segregation of a recessive “mh” (muscular hypertrophy) allele (Hanset and Michaux Gènèt. Sèl. Evol. 17:359-368 1985).

The same kind of experimental crosses were subsequently used to perform a whole genome scan using a microsatellite based marker map. To perform the linkage analysis, animals were classified as double-muscled or conventional. Very significant Logarithm of the Odds scores (lodscores) were obtained on chromosome 2 (>17), and multi-point linkage analysis positioned on the mh locus at the centromeric end of this chromosome, at [2]centimorgan from the nearest microsatellite marker: TGLA44. The corresponding chromosomal region accounted for all of the variance of the trait assumed to be fully penetrant in this experiment (Charlier et al. Mammalian Genome 6:788-792 1995).

Intensive breeding programs implemented over the last 50 years have created cattle breeds that are highly specialized in either milk production (e.g. Holstein-Friesian and Jersey) or meat production (e.g. Angus, Hereford, Charolais, Piedmontese, and Belgian Blue). Physiological antagonisms have indeed precluded combining superior abilities for both milk and meat production in the same animal. Despite its effectiveness, the resulting production system can be considered suboptimal because of poor carcass and milk yield of beef and diary cattle, respectively. Thus, the art lacks a production system that efficiently increases both milk yield and carcass yield in the same cattle population.

Furthermore, in humans, genes coding for some forms of muscular abnormalities have been isolated, e.g. muscular dystrophy. The present invention provides for the gene which regulates the development of skeletal muscle only, as opposed to other types of muscle, e.g. smooth or cardiac muscle. The present invention may provide an understanding of the role of the GDF-8 gene or its receptor in the regrowth of skeletal muscle in humans which only undergoes a hyperplasic response. The transgenic animals provided by the instant invention can be used as research tools to increase the understanding of the pathogenesis of disease in the muscular-skeletal system and to aid in the development of means to diagnosis and/or treat such diseases.

### SUMMARY OF THE INVENTION

The present inventors have identified and sequenced a gene (cDNA and genomic) encoding a bovine myostatin protein. The nucleic acid coding sequence is identified as SEQ ID NO:1 and the protein sequence is identified as SEQ ID NO:2. The genomic bovine sequence is identified as SEQ ID NO:54. A mutant gene (SEQ ID NO:3) in which the coding sequence lacks an 11-base pair consecutive sequence (SEQ ID NO:11) of the sequence encoding bovine protein having myostatin activity has been sequenced. It has been shown that cattle of the Belgian Blue breed homozygous for the mutant gene lacking myostatin activity are double-muscled. Other bovine mutations which lead to double-muscling have also been determined, being identified herein as nt419(de17-ins10), Q204X, E226X and C313Y, respectively.

In one aspect, the present invention thus provides a method for determining the presence of muscular hyperplasia in a mammal. The method includes obtaining a sample of material containing DNA from the mammal and ascertaining whether a sequence of the DNA encoding (a) a protein having the biologicalactivity of myostatin, is present, and whether a sequence encoding of the DNA encoding (b) an allelic protein lacking the activity of (a), is present. The absence of (a) and the presence of (b) indicates the presence of muscular hyperplasia in the mammal.

Of course, the mutation responsible for the lack of activity can be a naturally occurring mutation, as in the case for the Belgian Blue, Asturiana, Parthenaise or Rubia Gallega breeds, shown here.

The mammals of the instant invention are preferably, but not limited to, cattle.

There are several methods known for determining whether a particular nucleotide sequence is present in a sample. A common method is the polymerase chain reaction (PCR). A preferred aspect of the invention thus includes a step in which ascertaining whether a sequence of the DNA encoding (a) is present, and whether a sequence of the DNA encoding (b) is present includes amplifying the DNA in the presence of primers based on a nucleotide sequence encoding a protein having the biological activity of myostatin.

A primer of the present invention, used in PCR for example, is a nucleic acid molecule sufficiently complementary to the sequence on which it is based and of sufficient length to selectively hybridize to the corresponding portion of a nucleic acid molecule intended to be amplified and to prime synthesis thereof under in vitro conditions commonly used in PCR. Likewise, a probe of the present invention, is a molecule, for example a nucleic acid molecule of sufficient length and sufficiently complementary to the nucleic acid molecule of interest, which selectively binds under high or low stringency conditions with the nucleic acid sequence of interest for detection thereof in the presence of nucleic acid molecules having differing sequences.

In preferred aspects, primers are based on the sequences identified as SEQ ID NO:7 or SEQ ID NO:54.

In another aspect, the invention is a method for determining the presence of muscular hyperplasia in a mammal which includes obtaining a sample of material containing mRNA from the mammal. Such method includes ascertaining whether a sequence of the mRNA encoding (A) a protein having the biologicalactivity of myostatin, is present, and whether a sequence of the mRNA encoding (B) a protein at least partially encoded by a truncated nucleotide sequence corresponding to substantially the sequence of the mRNA and lacking the activity of (A), is present. The absence of (A) and the presence of (B) indicates the presence of muscular hyperplasia in the mammal.

The mRNA encoding (A) and the truncated sequence can correspond to alleles of DNA of the mammal.

Again, if an amplification method such as PCR is used in ascertaining whether a sequence of the mRNA encoding (A) is present, and whether a sequence of the mRNA encoding (B) is present, the method includes amplifying the mRNA in the presence of a pair of primers complementary to a nucleotide sequence encoding a protein having the biological activity of myostatin. Each such primer can contain a nucleotide sequence substantially complementary, for example, to the sequence identified as SEQ ID NO:7. The truncated sequence can contain at least 50 consecutive nucleotides substantially corresponding to 50 consecutive nucleotides of SEQ ID NO:7, for example.

In another aspect, the invention is a method for determining the presence of muscular hyperplasia in a mammal which includes obtaining a tissue sample containing mRNA of the mammal and ascertaining whether an mRNA encoding a mutant type myostatin protein lacking the biologicalactivity of myostatin is present. The presence of such an mRNA encoding a mutant type myostatin protein indicates the presence of muscular hyperplasia in the mammal.

In another aspect, the invention thus provides a method for determining the presence of muscular hyperplasia in a bovine animal. The method includes obtaining a sample of material containing DNA from the animal and ascertaining whether DNA having a nucleotide sequence encoding a protein having the biological activity of myostatin is present. The absence of DNA having such a nucleotide sequence indicates the presence of muscular hyperplasia in the animal. Ascertaining whether DNA having a nucleotide sequence encoding a protein having the biological activity of myostatin can include amplifying the DNA in the presence of primers based on a nucleotide sequence encoding a protein having the biological activity of myostatin.

In particular, the method can be carried out using a sample from an animal in which such a bovine animal not displaying muscular hyperplasia is known to have a nucleotide sequence which is capable of hybridizing with a nucleic acid molecule having the sequence identified as SEQ ID NO:1 under stringent hybridization conditions.

It is possible that ascertaining whether DNA having a nucleotide sequence encoding a protein having the biologicalactivity of myostatin is present includes amplifying the DNA in the presence of primers based on a nucleotide sequence encoding the N-terminal and the C-terminal, respectively, of the protein having the biological activity of myostatin.

Primers, say first and second primers, can be based on first and second nucleotide sequences encoding spaced apart regions of the protein, wherein the regions flank a mutation known to naturally occur and which when present in both alleles of such an animal results in muscular hyperplasia.

It can also be that DNA of such an animal not displaying muscular hyperplasia contains a nucleotide sequence which hybridizes under stringent conditions with a nucleotide sequence encoding a protein having a sequence identified as SEQ ID NO:2 and the coding sequence of DNA of such an animal displaying muscular hyperplasia is known to contain an 11-base pair deletion beginning at base pair number 821 of the coding sequence, and said first primer is selected to be upstream of the codon encoding glutamic acid number 275 and the second primer is selected to be downstream of the codon encoding aspartic acid number 274.

Also, a DNA of such an animal not displaying muscular hyperplasia might contain a nucleotide sequence which hybridizes under stringent conditions with a nucleotide sequence encoding a protein having a sequence identified as SEQ ID NO:2. The coding sequence of DNA of such an animal displaying muscular hyperplasia might be known to contain an 11-base pair deletion beginning at base pair number 821. A primer can be selected to span the nucleotide sequence including base pair numbers 820 and 821 of the DNA sequence containing the deletion.

The animal can be of the Belgian Blue breed.

In a particular aspect, ascertaining whether DNA having a nucleotide sequence encoding a protein having the biological activity of myostatin is present includes amplifying the DNA in the presence of a primer containing at least a portion of a mutation known to naturally occur and which when present in both alleles of a said animal results in muscular hyperplasia.

In another aspect, the invention is a method for determining the presence of muscular hyperplasia in a bovine animal which includes obtaining a sample of the animal containing mRNA and ascertaining whether an mRNA encoding a protein having the biological activity of myostatin is present in the sample. The absence of said mRNA indicates the presence of said muscular hyperplasia in the animal.

A sample containing mRNA is preferably, but not limited to, skeletal muscle tissue.

In a particular aspect, the invention is a method for determining the presence of double-muscling in a bovine animal, involving obtaining a sample of material containing DNA from the animal and ascertaining whether the DNA contains the nucleotide sequence identified as SEQ ID NO:11 in which the absence of the sequence indicates double-muscling in the animal.

The animal is preferably of, but not limited to, the Belgian Blue breed.

In another aspect, the invention is a method for determining the myostatin genotype of a mammal, as may be desirable to know for breeding purposes. The method includes obtaining a sample of material containing nucleic acid of the mammal, wherein the nucleic acid is uncontaminated by heterologous nucleic acid; ascertaining whether the sample contains a(i) nucleic acid molecule encoding a protein having the biological activity of myostatin; and ascertaining whether the sample contains an (ii) allelic nucleic acid molecule encoding a protein lacking the biological activity of myostatin. The mammal can be bovine.

In another aspect, the subject is human and (i) includes a nucleic acid sequence substantially homologous (in the sense of identity) with the sequence identified as SEQ ID NO:7.

The invention includes a method of increasing muscle mass of a mammal having muscle cells in which myostatin is expressed, the method comprising administering to the mammal an effective amount of a nucleic acid molecule substantially complementary to at least a portion of mRNA encoding myostatin and being of sufficient length to sufficiently reduce expression of the myostatin to increase the muscle mass. In a particularly preferred aspect, the mammal is bovine.

In another embodiment, the invention is a method of increasing muscle mass of a mammal, including administering to the mammal an effective amount of a nucleic acid molecule having ribozyme activity and a nucleotide sequence substantially complementary to at least a portion of mRNA encoding myostatin and being of sufficient length to bind selectively thereto to sufficiently reduce expression of the myostatin so as to increase the muscle mass.

The invention includes a diagnostic kit, for determining the presence of muscular hyperplasia in a mammal from which a sample containing DNA of the mammal has been obtained. The kit includes first and second primers for amplifying the DNA, the primers being complementary to nucleotide sequences of the DNA upstream and downstream, respectively, of a mutation in the portion of the DNA encoding myostatin which results in muscular hyperplasia of the mammal, wherein at least one of the nucleotide sequences is selected to be from a non-coding region of the myostatin gene. This kit can also include a third primer complementary to a naturally occurring mutation of a coding portion of the myostatin gene.

A particular diagnostic kit, for determining the genotype of a sample of mammalian genetic material, particularly bovine material, includes a pair of primers for amplifying a portion of the genetic material corresponding to a nucleotide sequence which encodes at least a portion of a myostatin protein, wherein a first of the primers includes a nucleotide sequence sufficiently complementary to a mutation of SEQ ID NO:1 to prime amplification of a nucleic acid molecule containing the mutation, the mutation being selected from a group of mutations resulting from: (a) deletion of 11 nucleotides beginning at nucleotide 821 of the coding portion of SEQ ID NO:1; (b) deletion of 7 nucleotides beginning at nucleotide 419 of the coding sequence and insertion of the sequence AAGCATACAA (SEQ ID NO:55) in place thereof; (c) deletion of nucleotide 204 of the coding sequence and insertion of T in place thereof; (d) deletion of nucleotide 226 of the coding sequence and insertion of T in place thereof; and (e) deletion of nucleotide 313 of the coding sequence and insertion of A in place thereof; and combinations thereof. The second of the pair of primers is preferably located entirely upstream or entirely downstream of the selected mutation or mutations. In one kit, a first said primer spans mutation (a) and further comprising a third primer which is sufficiently complementary to the nucleotide sequence identified as SEQ ID NO:11 to prime amplification of a nucleic acid molecule containing SEQ ID NO:11. In another (or the same kit), a first said primer is sufficiently complementary to the inserted sequence of mutation (b) to prime amplification of a nucleic acid molecule containing mutation (b) and further comprising a third primer which is sufficiently complementary to the sequence corresponding to the 7 nucleotide deletion of mutation (b) to prime amplification of a nucleic acid molecule containing the 7 nucleotide deletion of mutation (b). In another (or the same kit), a first said primer spans mutation (c) and further comprising a third primer which is sufficiently complementary to the sequence spanning the corresponding region lacking the mutation (c) to prime amplification of a nucleic acid molecule lacking mutation (c). In another (or the same kit), a first said primer spans mutation (d) and further comprising a third primer which is sufficiently complementary to the sequence spanning the corresponding region lacking mutation (d) to prime amplification of a nucleic acid molecule lacking mutation (d). In another (or the same kit), a first said primer spans mutation (e) and further comprising a third primer which is sufficiently complementary to a sequence spanning the corresponding region lacking mutation (e) to prime amplification of a nucleic acid molecule lacking mutation (e).

The invention includes a purified protein having the biological activity of myostatin, and having an amino acid sequence identified as SEQ ID NO:2, or a conservatively substituted variant thereof. The invention includes a purified bovine protein having the biological activity of myostatin or a purified human protein having the biologicalactivity of myostatin.

The invention includes an isolated nucleic acid molecule encoding a foregoing protein. Particularly, the invention includes an isolated nucleic acid molecule comprising a DNA molecule having the nucleotide sequence identified as SEQ ID NO:1 or SEQ ID NO:3 or SEQ ID NO:7 or which varies from the sequence due to the degeneracy of the genetic code, or a nucleic acid strand capable of hybridizing with at least one said nucleic acid molecule under stringent hybridization conditions.

The invention includes isolated mRNA transcribed from DNA having a sequence which corresponds to a nucleic acid molecule of the invention.

The invention includes an isolated DNA in a recombinant cloning vector and a microbial cell containing and expressing heterologous DNA of the invention.

The invention includes a transfected cell line which expresses a protein of the invention.

The invention includes a process for producing a protein of the invention, including preparing a DNA fragment including a nucleotide sequence which encodes the protein; incorporating the DNA fragment into an expression vector to obtain a recombinant DNA molecule which includes the DNA fragment and is capable of undergoing replication; transforming a host cell with recombinant DNA molecule to produce a transformant which can express the protein; culturing the transformant to produce the protein; and recovering the protein from resulting cultured mixture.

The invention includes a method of inhibiting myostatin so as to induce increased muscle mass in a mammal, comprising administering an effective amount of an antibody to myostatin to the mammal.

The invention includes a method of increasing muscle mass in a mammal, by raising an autoantibody to the myostatin in the mammal. Raising the autoantibody can include administering a protein having myostatin activity to the mammal.

The invention includes a method of increasing muscle mass in a mammal including administering to the mammal an effective amount of an antisense nucleic acid or oligonucleotide substantially complementary to at least a portion of the sequence identified as SEQ ID NO:1 or SEQ ID NO:5, or SEQ ID NO:7. The portion can be at least 5 nucleotide bases in length or longer. The mammal can be a bovine and the sequence can be that identified as SEQ ID NO:l.

The invention includes a method of inhibiting production of myostatin in a mammal in need thereof, including administering to the mammal an effective amount of an antibody to the myostatin.

The invention includes a probe containing a nucleic acid molecule sufficiently complementary with a sequence identified as SEQ ID NO:1, or its complement, so as to bind thereto under stringent conditions. The probe can be a sequence which is between about 8 and about 1195 nucleotides in length.

The invention includes a primer composition useful for detection of the presence of DNA encoding myostatin in cattle. The composition can include a nucleic acid primer substantially complementary to a nucleic acid sequence encoding a bovine myostatin. The nucleic acid sequence can be that identified as SEQ ID NO:1.

The invention includes a method for identifying a nucleotide sequence of a mutant gene encoding a myostatin protein of a mammal displaying muscular hyperplasia. The method includes obtaining a sample of material containing DNA from the mammal and probing the sample using a nucleic acid probe based on a nucleotide sequence of a known gene encoding myostatin in order to identify a nucleotide sequence of the mutant gene. In a particular approach, the probe is based on a nucleotide sequence identified as SEQ ID NO:1, SEQ ID NO:5 or SEQ ID NO:7. Preferably, the probe is at least 8 nucleotides in length. The step of probing the sample can include exposing the DNA to the probe under hybridizing conditions and further comprising isolated hybridized nucleic acid molecules. The method can further include the step of sequencing isolated DNA. The method can include the step of isolating and sequencing a cDNA or mRNA encoding the complete mutant myostatin protein. The method can include a step of isolating and sequencing a functional wild type myostatin from the mammal not displaying muscular hyperplasia.

The method can include comparing the complete coding sequence of the complete mutant myostatin protein with, if the coding sequence for a functional wild type myostatin from such a mammal is previously known, (1) the known sequence, or if the coding sequence for a functional myostatin from such a mammal is previously unknown, (2) the sequence(s) determined according to the invention, to determine the location of any mutation in the mutant gene.

The invention includes a primer composition useful for the detection of a nucleotide sequence encoding a myostatin containing a first nucleic acid molecule based on a nucleotide sequence located upstream of a mutation determined according to a method of the invention and a second nucleic acid molecule based on a nucleotide sequence located downstream of the mutation.

A probe of the invention can include a nucleic acid molecule based on a nucleotide sequence spanning a mutation determined according to the invention.

The invention includes an antibody to a protein encoded by a nucleotide sequence identified as SEQ ID NO:1, SEQ ID NO:3 or SEQ ID NO:7, or other protein of the present invention.

The invention includes a transgenic mammal, usually non-human, having a phenotype characterized by muscular hyperplasia, said phenotype being conferred by a transgene contained in the somatic and germ cells of the mammal, the transgene encoding a myostatin protein having a dominant negative mutation. The transgenic animal can be male and the transgene can be located on the Y chromosome. The mammal can be bovine and the transgene can be located to be under the control of a promoter which is normally a promoter of a myosin gene.

Another transgenic mammal, usually non-human, has a phenotype characterized by muscular hyperplasia, in which the phenotype is conferred by a transgene having a sequence antisense to that encoding a myostatin protein of the mammal. The mammal can be a male bovine and the transgene can be located on the Y chromosome. The transgene can further include a sequence which when transcribed obtains an RNA having ribozyme or siRNA (small interfering RNA) activity.

A transgenic non-human mammal of the invention having a phenotype characterized by muscular hyperplasia, can have the phenotype inducible and conferred by a myostatin gene flanked by loxP sites and Cre transgene under the dependence of an inducible promoter.

A transgenic non-human mammal of the invention having a phenotype characterized by muscular hyperplasia, can have the phenotype inducible and conferred by a myostatin gene flanked by loxP sites and Cre transgene located on the Y chromosome.

The invention includes a method for determining whether a sample of mammalian genetic material is capable of conferring a phenotype characterized by muscular hyperplasia, comprising ascertaining whether the genetic material contains a nucleotide sequence encoding a protein having the biological activity of myostatin, wherein the absence of said sequence indicates the presence of muscular hyperplasia in the animal.

An important objective of the instant invention is to provide a transgenic non-human male mammal exhibiting muscular hypertrophy, most particularly, but not limited to, a transgenic bovine.

Another important objective of the instant invention is to provide a method for producing a transgenic non-human male mammal exhibiting muscular hypertrophy, most particularly, but not limited to, a transgenic bovine.

It is also an objective of the invention to provide the embryonic stem cells or somatic cells for nuclear transfer necessary to produce a transgenic non-human male mammal exhibiting muscular hypertrophy. The somatic cells are preferably, but not limited to, fetal fibroblasts.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE FIGURES

This patent or application file contains at least one drawing executed in color. Copies of this patent or patent application publication with color drawing(s) will be provided by the Office upon request and payment of the necessary fee.

In describing particular aspects of the invention, reference is made to the accompanying drawings, in which:

FIG. 1 is a schematic summary of genetic, physical and comparative mapping information around the bovine locus. A multi-point lodscore curve obtained for the mh locus with respect to the microsatellite marker map is shown. Markers that were not informative in the pedigree used are shown between brackets; their map position is inferred from published mapping data. Markers and the YACs from which they were isolated are connected by arrows. The Rh-map of the relevant section of human HSA2 is shown, with the relative position in cR of the EST's used. Stippled lines connect microsatellite and Type I markers with their respective positive YACs. YACs showing cross-hybridizing SINE-PCR products are connected by the red boxes.

FIG. 2A shows electropherograms obtained by cycle-sequencing the myostatin cDNA sequence from a double-muscled and a conventional animal, showing the nt821del(11) deletion (SEQ ID NO:11 ATGAACACTCC) in the double-muscled animal. The primers used to amplify the fragment encompassing the deletion from genomic DNA are spaced apart from the remaining nucleotides. The sequences shown In FIG. 2A are all part of SEQ ID NO:1 (positions 836-1022), SEQ ID NO:3 (positions 836-1007) and SEQ ID NO:54 (positions 5101-5287).

FIG. 2B shows the amino acid sequence of the murine (top row, SEQ ID NO:6, positions 1-376), bovine normal (middle row, SEQ ID NO:2, positions 22-375) and bovine nt821/del (11)(bottom row, SEQ ID NO:4, positions 20-286) allele. The putative site of proteolytic processing is boxed, while the nine conserved cysteines in the carboxy-terminal region are underlined. The difference between the normal an the nt821del (11) bovine allele is indicated by the double underlining.

FIG. 3 is a schematic representation of the bovine myostatin gene with position and definition of the identified DNA sequence polymorphisms. The “A” (clear) boxes correspond to the untranslated leader and trailer sequences (large diameter), and the intronic sequences (small diameter) respectively. The “B”, “C”, and “D” boxes correspond to the sequences coding for the leader peptide, N-terminal latency-associated peptide (LAP) and the bioactive carboxyterminal domain of the protein respectively. Small “e”, “f” and “g” arrows point towards the positions of the primers used for intron amplification, exon amplification and sequencing and exon sequencing respectively; the corresponding primer sequences are reported in Table 1. The positions of the identified DNA sequence polymorphisms are shown as “h”, “i” or “j” lines on the myostatin gene for silent, conservative and disrupting mutations respectively. Each mutation is connected via an arrow with a box reporting the details of the corresponding DNA sequence and eventually encoded peptide sequence. In each box, the variant sequence is compared with the control Holstein-Friesian sequence and differences are highlighted in color. Box F94L shows four sequences: first row, SEQ ID NO:1 (positions 317-334), SEQ ID NO:3 (positions 317-334) and SEQ ID NO:54 (positions 724-741), second row, SEQ ID NO:2 (positions 91-96) and SEQ ID NO:4(positions 91-96), third row, SEQ ID NO:56 (shows mismatch with sequences shown in the first row) and fourth row, SEQ ID NO:57 (shows mismatch with sequences shown in the second row). Box nt419 shows four sequences: first row, SEQ ID NO:1 (positions 458-479), SEQ ID NO:3 (positions 458-479) and SEQ ID NO:54 (positions 2691-2708), second row, SEQ ID NO:2 (positions 138-143) and SEQ ID NO:4 (positions 138-143), third row, SEQ ID NO:58 and fourth row, SEQ ID NO:2 (positions 138-139) and SEQ ID NO:4 (positions 138-139). Box nt748-78 shows two sequences: first row, SEQ ID NO:54 (positions 4973-4989) and second row, SEQ ID NO:59 (shows mismatch with the sequence shown in the first row). Box nt374-51 shows two sequences, first row, SEQ ID NO:60 and second row SEQ ID NO:61. Box Q204X shows four sequences: first row, SEQ ID NO:1 (positions 647-664), SEQ ID NO:3 (positions 647-664) and SEQ ID NO:54 (positions 2880-2897), second row, SEQ ID NO:2 (positions 201-206) and SEQ ID NO:4 (positions 201-206), third row, third row, SEQ ID NO:62 (shows mismatch with sequence shown in the first row) and fourth row, SEQ ID NO:2 (positions 201-203) and SEQ ID NO:4 (positions 201-203). Box nt821 shows four sequences: first row, SEQ ID NO:1 (positions 860-880) and SEQ ID NO:54 (positions 5101-5125), second row, SEQ ID NO:2 (positions 272-278) and SEQ ID NO:4 (positions 272-278), third row, SEQ ID NO:3 (positions 860-868) and fourth row, SEQ ID NO:4 (positions 272-274). Box nt374-16 shows two sequences: first row, SEQ ID NO:54 (positions 2631-2645) and second row, SEQ ID NO:63 (shows mismatch with sequence shown in the first row). Box nt414 shows four sequences: first row, SEQ ID NO:1 (positions 449-466), SEQ ID NO:3 (positions 449-466) and SEQ ID NO:54 (positions 724-741), second row, SEQ ID NO:2 (positions 135-140) and SEQ ID NO:4 (positions 135-140), third row, SEQ ID NO:64 (shows mismatch with sequence shown in first row) and fourth row SEQ ID NO:2 (positions 135-140) and SEQ ID NO:4 (positions 135-140). Box E226X shows four sequences, first row, SEQ ID NO:1 (positions 713-730), SEQ ID NO:3 (positions 713-730) and SEQ ID NO:54 (positions 2946-2963), second row, SEQ ID NO:2 (positions 223-228) and SEQ ID NO:4 (positions 223-228), third row, SEQ ID NO:65 (shows mismatch with sequence shown in the first row) and fourth row, SEQ ID NO:2 (positions 223-225) and SEQ ID NO:4 (positions 223-225). Box 313Y shows four sequences: first row, SEQ ID NO:1 (positions 974-991) and SEQ ID NO:54 (positions 5239-5256), second row, SEQ ID NO:2 (positions 310-315), third row, SEQ ID NO:66 (shows mismatch with sequence shown in the first row) and fourth row, SEQ ID NO:67 (shows mismatch with sequence shown in the second row).

FIG. 4 shows the distribution of identified mutations in the various breeds examined. The order of the myostatin mutations correspond to FIG. 3. All analyzed animals were double-muscled except for the two Holstein-Friesian and two Jerseys used as controls (column 1).

FIG. 5 is a schematic representation of the targeting strategy used for producing a transgenic non-human male mammal exhibiting muscular hypertrophy.

FIG. 6 shows data demonstrating the integration of the transgene on the Y chromosome for both the R1-UP-neotk (left) and R1-TSPY-neotk (right) clones.

FIG. 7A is an analysis of transgene expression in the F1-UP-LAP and F1-TSPY-LAP transgenic lines by Northern blotting technique.

FIG. 7B is an analysis of transgene expression in the BC-UP-LAP and BC-TSPY-LAP transgenic lines by Northern blotting technique.

FIG. 8 is a chart showing the cumulative frequency distribution of quadriceps femoris myofiber diameter in males and females of the BC-CONT (blue), BC-UP-LAP (red) and BC-TSPY-LAP (green) lines.

FIG. 9 shows the data resulting from the experiments carried out to screen for ES clones having undergone proper gene targeting on the Y chromosome of the pPNTdloxUP construct.

FIG. 10 shows the data resulting from the experiments carried out to screen for ES clones having undergone proper gene targeting on the Y chromosome of the pPNTdloxTSPY construct.

FIG. 11 shows the data resulting from the experiments carried out to screen for R1-UP-neotk ES clones having undergone proper recombinase-mediated cassette exchange (RMCE) with the mDAFdloxLAP vector.

FIG. 12 shows the data resulting from the experiments carried out to screen for R1-TSPy-neotk ES clones having undergone proper recombinase-mediated cassette exchange (RMCE) with the mDAFdloxLAP vector.

FIG. 13 is graph of the growth curves over seven weeks (week 4-week 10) of BC-CONT, BC-UP-LAP, and BC-TSPY-LAP animals sorted by sex.

FIG. 14 presents data characterizing the BAC clone containing bovine Y-specific sequences useful as a gene targeting site to produce transgenic cattle.

### DEFINITIONS

The following list defines terms, phrases and abbreviations used throughout the specification. Although the terms, phrases and abbreviations are listed in the singular tense, this list is intended to encompass all grammatical forms.

As used herein, the term “double-muscling” describes an increase in skeletal muscle mass due to loss of the biological function of myostatin protein. Double-muscling can result from muscular hyperplasia and/or hypertrophy.

As used herein, the term “hyperplasia” refers to an abnormal increase in the number of cells in an organ and/or tissue resulting in enlargement of the organ and/or tissue.

As used herein, the term “hypertrophy” refers to the enlargement of an organ and/or tissue resulting from an increase in the size of the individual cells of the organ and/or tissue.

As used herein, the abbreviation “MSTN” refers to myostatin. Myostatin a protein of the transforming growth factor-(β (TGF-β)superfamily that acts as a negative regulator of skeletal muscle mass (Lee and McPherron PNAS 98:169306-9311 2001). Myostatin is also called growth differentiation factor-8 (GDF-8). Disruption of the myostatin gene in mice double skeletal muscle mass (McPherron et al. Nature 387:83-90 1997). Conversely, systemic over-expression of the myostatin gene leads to a wasting syndrome characterized by extensive muscle loss (Zimmers et al. Science 296:1486-1488 2002).

As used herein, the abbreviation “LAP” refers to the latency-associated peptide or myostatin propeptide. Myostatin protein as purified from mammalian cells consists of a noncovalently held complex of the N-terminal propeptide and a disulfide-linked dimer of C-terminal fragments. This C-terminal dimer is held in an inactive complex with the propeptide and other proteins. Thus, the myostatin propeptide or LAP as an inhibitory/inactivating effect on the biological function of myostatin (Lee and McPherron PNAS 98:169306-9311 2001).

As used herein, the term “dominant-negative effect” involves proteins that act as dimers and results from the ability of a mutated/inactive subunit to dimerize with the active subunit and thus inactivate the normal protein. The transgenic non-human male mammals of the instant invention are produced using the latency-associated peptide as a dominant-negative means to repress endogenous myostatin activity.

As used herein, the phrase “protein having biological activity of myostatin” means that a protein or any portion thereof is capable of the biological function of myostatin.

As used herein, the term “genotype” refers to the entire genetic constitution of an organism; i.e. genes of an organism, both dominant and recessive.

As used herein, the term “phenotype” refers to the observable characteristics of an individual resulting from the interaction of the individual's genotype with the environment. For example, the phenotype of double-muscling is seen in an animal having within its' genotype the nt821(del11) mutation in the gene encoding for myostatin.

As used herein, the term “allele” refers to an alternative form of a gene and/or any one of several mutational forms. The nt821(del11) mutation and the normal are both alleles of the MSTN gene.

As used herein, the term “microsatellite” refers to a segment of DNA about 2 to 6 nucleotides in length which is tandomly repeated.

As used herein, the term “promoter” refers to a sequence at the 5′ end of a gene which binds DNA polymerase and/or transcription factors to regulate expression of the gene. Promoters can be tissue-specific.

As used herein, the term “transgenic” refers to a cell and/or animal having a genome into which genetic material from a different organism has been artificially introduced. The transgenic animals of the instant invention contain DNA for a myostatin trans-repressor that when expressed inactivates endogenous myostatin.

As used herein, the phrase “naturally occurring mutation” refers to a mutation in genetic material that is not artificially introduced.

As used herein, the abbreviation “BBB” refers to the Belgian Blue Breed of cattle. The BBB of cattle express naturally occurring myostatin-inactivating mutations.

As used herein, the term “dystocia” refers to a slow and/or difficult labor. The BBB of cattle often experience dystocia due to the double-muscling phenotype.

As used herein, the term “bovine” means of or relating to an animal of the cattle group, including buffalo and bison.

As used herein, the term “murine” means of or relating to rodents, including mice and rats.

As used herein, the abbreviation “ES cell” refers to an embryonic stem cell which is a pluripotent, balstocyst-derived cell that retains the developmental potential to differentiate into all somatic and germ cell lineages (Robertson, E. J. Trends in Genetics 2:9-13 1986). The ES cells of the instant invention are preferably, but not limited to, murine ES cells.

As used herein, the term “expression” includes transcription and translation.

As used herein, the term “heterologous” or “foreign” refers to nucleic acid and/or amino acid sequences not naturally occurring in the cell/organism of interest. Heterologous sequences may also be found in a location or locations in the genome that differs from that in which it occurs in nature.

As used herein, the term “endogenous” refers to nucleic acid and/or amino acid sequences naturally occurring in the cell/organism of interest.

As used herein, the term “recombinant” refers to genetic material, cells and/or organisms that have been genetically modified; for example, by addition of heterologous genetic material or modification of the endogenous genetic material.

As used herein, the term “isolated” or “purified” refers to nucleic acid and/or amino sequences that have been removed from at least one component with which it is naturally associated. For example, an isolated protein is substantially free of cellular material or culture medium when produced by molecular biological techniques.

As used herein, the term “vector” refers to a polynucleotide construct designed for transduction and/or transfection of one or more cell types.

As used herein, the phrase “operably linked” when referring to a transcriptional regulatory element and a coding sequence is intended to mean that the regulatory sequence is associated with the coding sequence in such a manner as to facilitate transcription of the coding sequence.

As used herein, the term “homologous recombination” refers to the exchange of DNA fragments between two DNA molecules or chromatids at the site of homologous nucleotide sequences.

As used herein, the term “gene targeting” refers to a type of homologous recombination that occurs when a fragment of genomic DNA is introduced into a mammalian cell and that fragment locates and recombines with endogenous homologous sequences. The first step of the method for producing the transgenic non-human male mammals of the invention is carried out by gene targeting.

As used herein, the term “cre recombinase” refers to a specific DNA recombinase which recognizes a specific nucleotide sequence (lox site) and conducts complete processing, including strand cleavage, strand exchange and ligation of each strand within the site. A cre gene can be isolated from the E. coli bacteriophage P1 by methods known in the art (Abremski et al. Cell 32:1301-1311 1983; Sternberg et al. Journal of Molecular Biology 150:467-486 1981). The use of a cre/lox system provides specific gene expression at a specifically desired time.

As used herein, the term “lox site” refers to a specific sequence of nucleotides recognized by a cre recombinase. There are several different lox sites, including, but not limited to, loxP, loxB, loxl, loxR and loxC2. These sequences can be isolated from the E. coli bacteriophage P1 by methods known in the art (Abremski et al. Cell 32:1301-1311 1983; Sternberg et al. Journal of Molecular Biology 150:467-486 1981). The term “flox” means to flank a portion of a nucleotide sequence (gene) with one or more lox sites.

As used herein, the abbreviation “RMCE” refers recombinase-mediated cassette exchange, a method for specific expression of genetic material at a given time mediated by the cre recombinase. The second step of the method for producing the transgenic non-human male mammals of the invention is carried out by RMCE.

As used herein, the term “FISH” refers to fluorescent in situ hybridization, a technique useful for identifying whole chromosomes or parts of chromosomes using fluorescent-tagged DNA probes.

As used herein, the abbreviation “TSPY” refers to the testis-specific protein Y-encoded pseudogene. A pseudogene is a segment of DNA that resembles a gene but lacks a genetic function. The TSPY gene was chosen as the targeting site on the murine Y chromosome for the first step of the claimed method for producing the transgenic male mouse of the instant invention.

As used herein, the abbreviation “MLC” refers to the myosin light chain, specific to skeletal muscle. The regulatory elements (promoter and enhancer) of the MLC gene are used to control the expression of the LAP in the transgenic non-human male mammals of the instant invention.

As used herein, the abbreviation “SV40” refers to the simian virus 40. The regulatory elements (small tumor antigen intron and polyadenylation signal) of the SV40 genome are used to control the expression of the LAP in the transgenic non-human male mammals of the instant invention.

As used herein, the term “polyadenylation signal” refers to a sequence (AATAAA) near the 3′ end of a primary transcript which signals that a polyadenine tail be added to the newly formed transcript. A polyadenine tail can be several hundred nucleotides long and seems to play a role in the stability of mRNA.

As used herein, the abbreviation “BAC” refers to a bacterial artificial chromosome. A BAC is a cloning vector based on E. coli F-factor replicon. Large segments of DNA from another species are cloned into bacterial DNA to form BACs.

As used herein, the term “CpG islands” refers to areas of multiple CG (cytosine and guanine) repeats in a nucleic acid molecule.

As used herein, the term “ESTs” refers to short segments of incompletely sequenced cDNA; allow for design of a PCR reaction which may be used to test for the presence of the cDNA.

As used herein, the abbreviation “UP” means upstream.

As used herein, the abbreviation “BC” means backcross.

As used herein, the abbreviation “CONT” means control.

### DETAILED DESCRIPTION OF THE INVENTION

The method used for isolating genes which cause specific phenotypes is known as positional candidate cloning. It involves: (i) the chromosomal localization of the gene which causes the specific phenotype using genetic markers in a linkage analysis; and (ii) the identification of the gene which causes the specific phenotype amongst the “candidate” genes known to be located in the corresponding region. Most of the time these candidate genes are selected from available mapping information in humans and mice.

The tools required to perform the initial localization (step(i) above) are microsatellite marker maps, which are available for livestock species and are found in the public domain (Bishop et al., 1994; Barendse et al., 1994; Georges et al., 1995; and Kappes, 1997). The tools required for the positional candidate cloning, particularly the YAC libraries, (step (ii) above) are partially available from the public domain. Genomic libraries with large inserts constructed with Yeast Artificial Chromosomes (“YAC”) are available in the public domain for most livestock species including cattle. When cross-referencing the human and mouse map, it is necessary to identify the positional candidate, which is available at low resolution but needs to be refined in every specific instance to obtain the appropriate level of high resolution. A number of original strategies are described herein to achieve this latter objective. For general principles of positional candidate cloning, see Collins, 1995 and Georges and Andersson, 1996.

In order to allow for cross-referencing between the bovine and human gene map as part of the positional candidate cloning approach, HSA2q31-32 (map of the long arm of human chromosome 2, cytogenetic bands q31-32) and BTA2q12-22 (map of the arm of bovine chromosome 2, cytogenetic bands q12-22) were integrated on the basis of coincidence, bovine YAC's as described below.

Using a previously described experimental [(normal x double-muscled) x double-muscled] backcross population comprising 108 backcross individuals, the mh locus was recently mapped by linkage analysis to the centromeric tip of bovine chromosome 2 (BTA2), at 3.1 centiMorgan proximal from the last marker on the linkage map: TGLA44 (Charlier et al., 1995). It was also known from previous work that pro-α(III) collagen (Col3AI) was located in the same chromosomal region as the mh locus. Col3AI has been mapped to BTA2q12-22 by in situ hybridization (Solinas-Toldo et al., 1995), while a Col3AI RFLP marker was shown to be closely linked to TGLA44 (θ=2%)(Fisher et al., 1996). This identifies the region flanking Col3AI on the human map, i.e. HSA2q31-32, as the likely orthologous human chromosome segment. This assumption is compatible with data from Zoo-FISH experiments (Solinas-Toldo et al., 1995) as well as mapping data of Type I markers on somatic cell hybrids (O'Brien et al., 1993), which establish an evolutionary correspondence between segments of HSAq2 and BTA2.

In order to refine the correspondence between the HSA2q31-33 and BTA2q12-22 maps, Comparative Anchored Tagged Sequences or CATS, i.e. primer pairs that would amplify a Sequence Tagged Site or STS from the orthologous gene in different species (Lyons et al., 1996), were developed for a series of genes flanking Col3A1 on the human map and for which sequence information was available in more than one mammal. In addition to Col3AI, working CATS were obtained for α2(V) collagen (Col5A2), inositol polyphosphate-1 phosphatase (INPP1), tissue factor pathway inhibitor precursor (TFPI), titin (TTN), n-chimaerin (CHN), glutamate decarboxylase 67 (GAD1), Cytotoxic T-lymphocyte-associated protein 4 (CTLA4) and T-cell membrane glycoprotein CD28 (CD28). The corresponding primer sequences are given in Table 1.

TABLE 1
CATS

| | | |
|---|---|---|
| INPP1 | UP: 5′ CAGCAAAGTCCTTAATGGTAACAAGC 3′ | DN: 5′ GGGTCACTGAAGAAAACGTCCTG 3′ |
| COL3A1 | UP: 5′ CCCCATATTATGGAGATGAACCG 3′ | DN: 5′ AGTTCAGGATGGCAGAATTTCAG 3′ |
| COL5A2 | UP: 5′ GCAAACTGGGYGGRAGCAAGACC 3′ | DN: 5′ TTSTTCCTGGGCTTTTATTGAGAC 3′ |
| TFPI | UP: 5′ AAGCCWGATTTCTGCTTYTTGGAAG 3′ | DN: 5′ TGCCMAGGCAHCCRCCRTACTTGAA 3′ |
| TTN | UP: 5′ GGTCGTCCTACACCAGAAG 3′ | DN: 5′ GGTTGACATTGTCAAGAACAAG 3′ |
| CHN | UP: 5′ TCTCMAAAGTCGTCTGTGACAATC 3′ | DN: 5′ TGYTCRTTTTCTTTCAGAGTTGC 3′ |
| GAD1 | UP: 5′ RCTGGTCCTCTTCACCTCAGAAC 3′ | DN: 5′ ACATTGTCVGTTCCAAAGCCAAG 3′ |
| CTLA4 | UP: 5′ AGGTYCGGGTGACDGTGCTKC 3′ | DN: 5′ TGGRTACATGAGYTCCACCTTGC 3′ |
| CD28 | UP: 5′ AGCTGCARGTATWCCTACAAYCT 3′ | DN: 5′ GTYCCRTTGCTCYTCTCRTTGYC 3′ |

Microsatellite markers

| | | |
|---|---|---|
| TGLA44 | UP: 5′ AACTGTATATTGAGAGCCTACCATG 3′ | DN: 5′ CACACCTTAGCGACTAAACCACCA 3′ |
| BULGE27 | UP: 5′ CTACCTAACAGAATGATTTTGTAAG 3′ | DN: 5′ AGTGTTCTTGCCTAGAGAATCCCAG 3′ |
| BULGE23 | UP: 5′ ACATTCTCTCACCAATATGACATAC 3′ | DN: 5′ TAAGTCACCATTACATCCTTAGAAC 3 |
| BM81124 | UP: 5′ GCTGTAAGAATCTTCATTAAGCACT 3′ | DN: 5′ CCTGATACATGCTAAGGTTAAAAAC 3″ |
| BULGE28 | UP: 5′ AGGCATACATCTGGAGAGAAACATG 3′ | DN: 5′ CAGAGGAGCGTAGCAGGCTACCGTC 3′ |
| BULGE20 | UP: 5′ CAGCAGGTCTGTTGAAGTGTATCAG 3′ | DN: 5′ AGTGGTAGCATTCACAGGTAGCCAG 3′ |
| BM3627 | UP: 5′ CAGTCCATGGCACCATAAAG 3′ | DN: 5′ TCCGTTAGTACTGGCTAATTGC 3′ |
| ILSTS026 | UP: 5′ CTGAATTGGCTCCAAAGGCC 3′ | DN: 5′ AAACAGAAGTCCAGGGCTGC 3′ |
| INRA40 | UP: 5′ TCAGTCTCCAGGAGAGAAAAC 3′ | DN: 5′ CTCTGCCCTGGGGATGATTG 3′ |

Bovine Myostatin primers

| | |
|---|---|
| GDF8.19 | 5′ AATGTATGTTTATATTTACCTGTTCATG 3′ |
| GDF8.11 | 5′ ACAGTGTTTGTGCAAATCCTGAGAC 3′ |
| GDF8.12 | 5′ CAATGCCTAAGTTGGATTCAGGTTG 3′ |
| GDF8.25 | 5′ CTTGCTGTAACCTTCCCAGGACCAG 3′ |
| GDF8.15 | 5′ TCCCATCCAAAGGCTTCAAAATC 3′ |
| GDF8.21 | 5′ ATACTCWAGGCCTAYAGCCTGTGGT 3′ |

Reading from left to right and down the table, the sequences given in Table 1 are identified as SEQ ID NO: 12 to SEQ ID NO: 53, respectively.

These CATS were used to screen a 6-genome equivalent bovine YAC library by PCR using a three-dimensional pooling strategy as described by Libert et al., 1993. The same YAC library was also screened with all microsatellite markers available for proximal BTA2, i.e. TGLA44, BM81124, BM3627, ILSTS026, INRA40 and TGLA431 (Kappes et al., 1997).

Potential overlap between the YACs obtained with this panel of STS's was explored on the basis of common STS content, as well as cross-hybridization between SINE-PCR product from individual YACs. From this analysis, three independent YAC contigs emerged in the region of interest: (i) contig A containing microsatellites TGLA44, BM81124 and Type I marker INPP1; (ii) contig B containing Col3AI and Col5A2; and (iii) contig C containing microsatellite markers BM3627, ILSTS026 and INRA40, and Type I marker TFPl.

None of the available microsatellites mapped to contig B, therefore this cluster of YACs could not be positioned in cattle with respect to the other two contigs. Available mapping information in the human, however, allowed prediction of contig B's position between contigs A and C. To test this hypothesis, two new microsatellite markers were isolated from contig B, BULGE20 and BULGE28. BULGE20 proved to be polymorphic, allowing for genotyping of the experimental backcross population.

In addition, to increase the informativeness of the markers available for contig A, two new microsatellite markers were developed from this contig: BULGE23 and BULGE27. BULGE23 proved to be polymorphic and was used to type the same pedigree material.

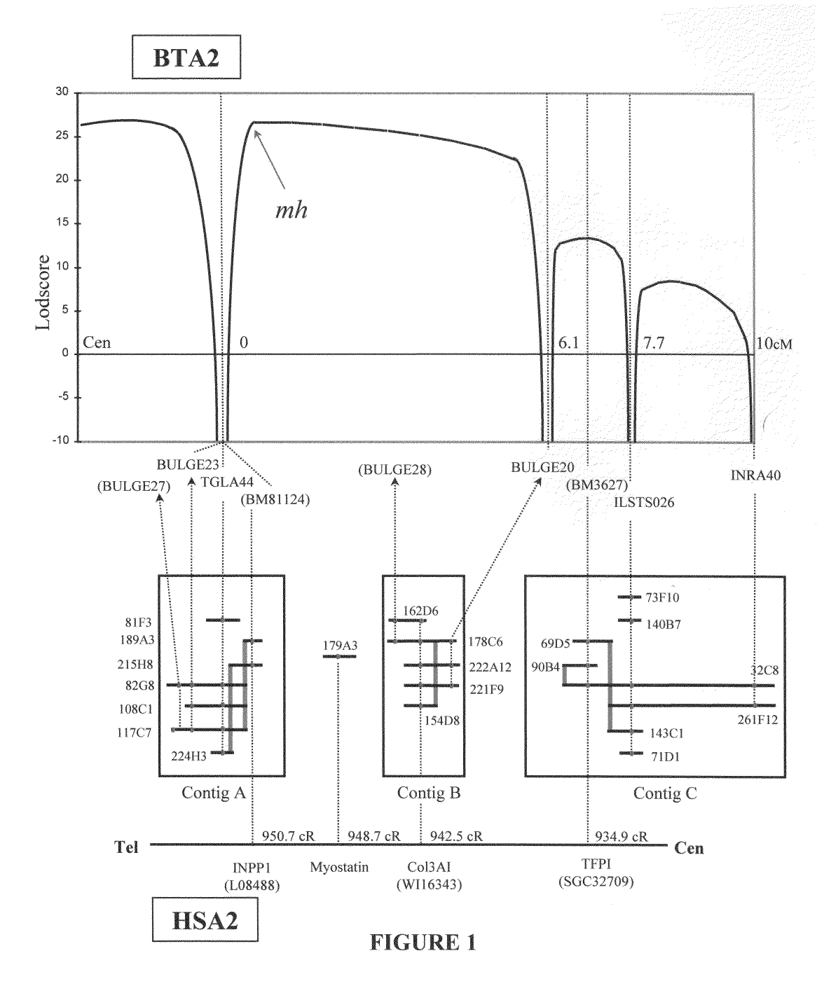

All resulting genotypes were used to construct a linkage map using the lLINK program (Lathrop and Lalouel, 1984). The following most likely order and sex-averaged recombination rates between adjacent markers was obtained: [TGLA44-(0%)-BULG23]-(6.1%)-BULG20-(1.6%)-ILSTS026-(2.3%)-INRA40-(7.1%)-TGLA431. The position of BULGE20 between TGLA44 and ILSTS026 confirmed the anticipated order of the three contigs. FIG. 1 summarizes the resulting mapping information.

A multi point linkage analysis was undertaken using LINKMAP, to position the mh locus with respect to the new marker map. Linkage analysis was performed under a simple recessive model, assuming full penetrance for mh/mh individuals and zero penetrance for the two other genotypes. The LOD score curve shown in FIG. 1 was obtained, placing the mh locus in the TGLA44-BULGE20 interval with an associated maximum LOD score of 26.4. Three backcross individuals were shown to recombine with the BULGE20 and distal markers, but not with TGLA44 and BULGE23, therefore placing the mh locus proximal from this marker. One individual, was shown to recombine with TGLA44 and BULGE23, but not with the more distal markers, therefore positioning the mh locus distal from TGLA44 and BULGE23. Given the relative position of these microsatellite markers with respect to INPP1 and Col3AI as deduced from the integration of the human and bovine map, these results indicated that the mh gene is likely located in a chromosome segment bounded by INPP1 and Col3AI.

Recently, McPherron et al. (1997) demonstrated that mice homozygous for a knock-out deletion of GDF-8 or myostatin, were characterized by a generalized increase in skeletal muscle mass. Using the published 2676 bp murine myostatin cDNA sequence (GenBank accession number U84005), a Tentative Human Consensus (THC) cluster in the Unigene database was identified which represented three cDNA clones (221299, 300367, 308202) and six EST (Expressed Sequence Tag) sequences (H92027, H92028, N80248, N95327, W07375, W24782). The corresponding THC covered 1296 by of the human myostatin gene, showing an homology of 78.1% with the murine sequence when averaged over the entire sequence, and 91.1% when considering only the translated parts of the human and murine genes (566 bp). This THC therefore very likely corresponds to the human orthologue of the murine myostatin gene.

Primers (5′-GGCCCAACTATGGATATATTTG-3′ (SEQ ID NO:9) and 5′-GGTCCTGGGAAGGTTACAGCA-3′ (SEQ ID NO:10)) were thus prepared to amplify a 272 by fragment from the second exon of human myostatin and used to genotype the whole-genome Genebridge-4 radiation hybrid panel (Walter et al., 1994). The RHMapper program (Slonim et al., unpublished) was used to position the myostatin gene with respect to the Whitehead/MIT framework radiation hybrid map, placing it at position 948.7 cR of the HSA2 map with an associated lodscore >3. Closer examination of the myostatin segregation vector and its confrontation with the vectors from all markers located in that region (Data Release 11.9, May 1997) showed it to be identical to EST SGC38239 placed on the Whitehead/MIT radiation hybrid map (Hudson et al., 1995) at position 946.8 cR of HSA2. This places the human myostatin gene on the RH-map in the interval between Col3AI (EST WI16343-942.5 cR) and INPP1 (EST L08488-950.2 to 951.2 cR) (FIG. 1). Myostatin therefore appeared as a very strong positional candidate for the mh gene.

To test the potential involvement of myostatin in the determinism of muscling in cattle, primer pairs were designed based on the available mouse and human myostatin sequence, with the objective to amplify the entire coding sequence from bovine cDNA using PCR (Polymerase Chain Reaction). Whenever possible, primers were therefore positioned in portions of the myostatin sequence showing 100% homology between mouse and human. Two primer pairs were identified that amplified what was predicted to represent 98.4% DNA fragments, respectively 660 (primers GDF8.19-GDF8.12) and 724 by (primers GDF8.11-GDF8.21) long. The expected DNA products were successfully amplified from cDNA generated from skeletal muscle of both a normal (homozygous +/+) (SEQ ID NO:1) and a double-muscled (homozygous mh/mh) (SEQ ID NO:3) animal, and cycle-sequences on both strands.

The nucleotide sequence corresponding to the normal allele presented 88.9% identity with the mouse myostatin sequence (SEQ ID NO:5) over a 1067 by overlap, and contained the expected open reading frame encoding a protein (SEQ ID NO:2) showing 92.9% identity in a 354 amino-acid overlap with mouse myostatin (SEQ ID NO:6). As expected for a member of the TGF superfamily, the bovine myostatin gene is characterized by a proteolytic processing site thought to mediate cleavage of the bioactive carboxy-terminal domain from the longer N-terminal fragment, and by nine cysteine residues separated by a characteristic spacing and suspected to be involved in intra- and inter-molecular disulfide bridges (McPherron and Lee, 1996).

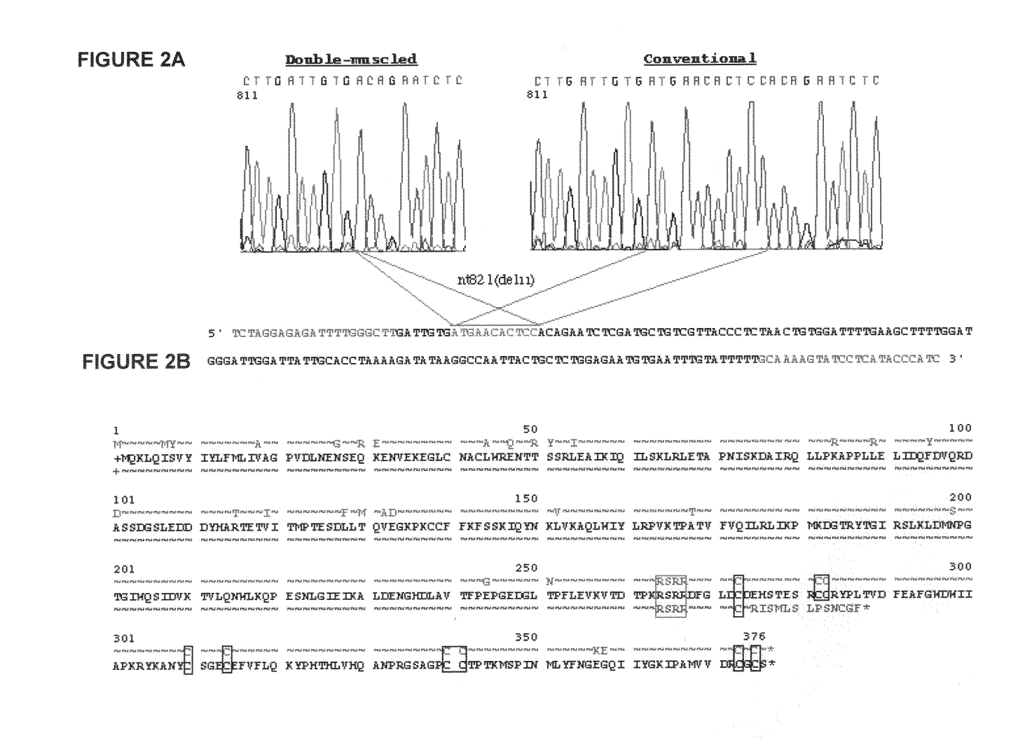

The nucleotide sequence obtained from the mh allele was identical to the normal allele over its entire length, except for an 11 bp deletion involving nucleotides 821 to 831 (counting from the initiation codon). This frame shifting deletion, occurring after the first cysteine residue of the carboxy-terminal domain, drastically disrupts the downstream amino-acid sequence and reveals a premature stop-codon after 13 amino acids, see FIG. 2. The amino acid sequence encoded by the mutant nucleic acid sequence is identified as SEQ ID NO:4. This mutation disrupts the bioactive part of the molecule and is therefore very likely to be the cause of the recessive double-muscling phenotype. Following conventional nomenclature, this mutation will be referred to as nt821(del11).

To further strengthen the assumption of the causality of this mutation, primer pairs flanking the deletion (FIG. 2) were prepared and the corresponding DNA segment from all animals from the experimental backcross population amplified. PCR was performed in the presence of dCTP³² in order to radioactively label the amplification product. Amplification products were separated on denaturing polyacrylamide gels and detected by autoradiography. A 188 by product would be expected for the normal allele and a 177 by product for the nt821(del11) allele. Correlation between phenotype and genotype was matched for the entire pedigree. All ten BBCB double-muscled sires were found to be homozygous for the nt821(del11) mutation, all 41 F1 females were heterozygous, while 53 double-muscled offspring were homozygous for the mutation, the remaining 55 conventional animals were heterozygous.

To examine the distribution of the nt821(del11) mutation in different conventional and double-muscled breeds, a cohort of 25 normal individuals were genotyped representing two dairy breeds (Holstein-Friesian, Red-and -White) and a cohort of 52 double-muscled animals representing four breeds (BBCB, Asturiana, Maine-Anjou and Piémontese). The results are summarized in Table 2. All dairy animals were homozygous normal except for one Red-and-White bull shown to be heterozygous. The occurrence of a small fraction of individuals carrying the mutation in dairy cattle is not unexpected as the phenotype is occasionally described in this breed. In BBCB and Asturiana, all double-muscled animals were homozygous for the nt821(del11) deletion, pointing towards allelic homogeneity in these two breeds. Double-muscled Maine-Anjou and Piémontese animals were homozygous “normal”, i.e. they did not show the nt821(del11) deletion but a distinct cysteine to tyrosine substitution (C313Y) in double-muscled Piédmontese animals identified by others (Kambadur et al., 1997) was discovered.

TABLE 2

| | | Genotype | | |
|---|---|---|---|---|
| | | | | nt821(del11)/ |
| Breed | Phenotype | +/+ | +/nt821(del11) | nt821(del11) |
| Belgian Blue | DM | | | 29 |
| Asturiana | DM | | | 10 |
| Piémontese | DM | 8 | | |
| Maine-Anjou | DM | 4 | | |
| Holstein-Friesian | Normal | 13 | | |
| Red-and-White | Normal | 12 | 1 | |

The entire coding sequence was also determined for the myostatin gene in double-muscled individuals from ten European cattle breeds and a series of mutation that disrupt myostatin function were identified.

The coding sequence of four control Holstein-Friesian and Jersey individuals was identical to the previously described wild-type allele (Grobet et al., 1997), further indicating that it was the genuine myostatin coding sequence being amplified, and not a non-functional pseudogene.

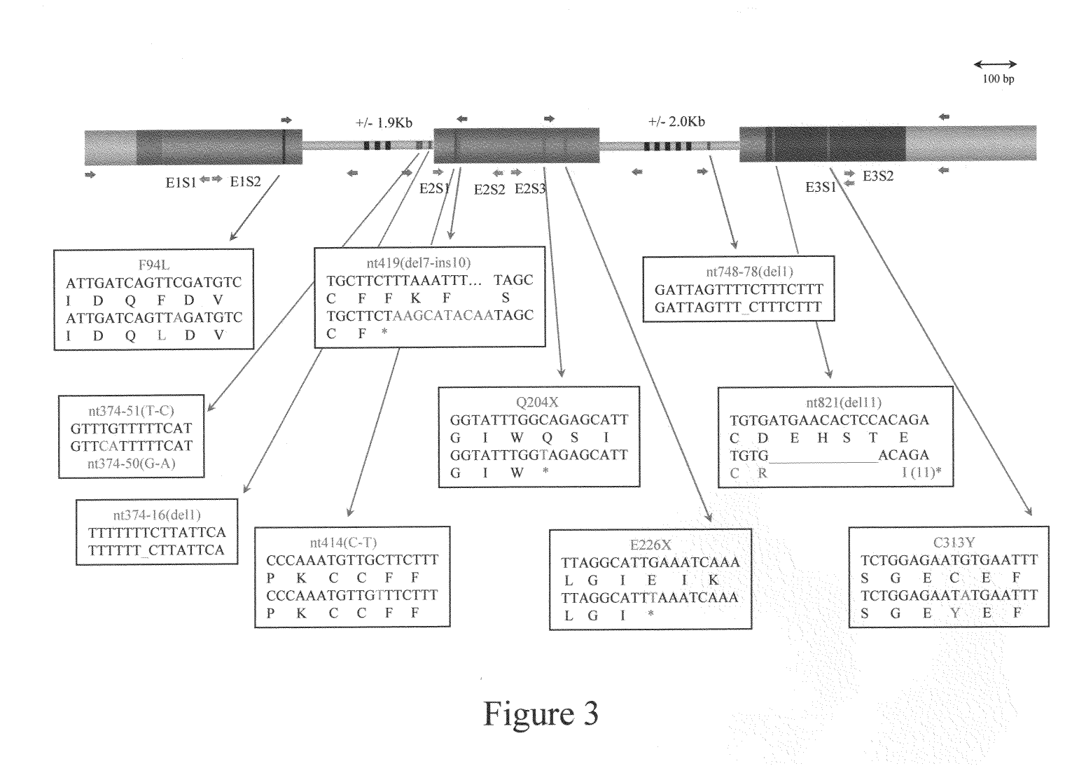

Amongst the 32 double-muscled animals, seven DNA sequence variants within the coding region were found, as summarized in FIG. 3.

In addition to the nt821(del11) mutation in the third exon, described above, four new mutations that would be expected to disrupt the myostatin function were found. An insertion/deletion at position 419 counting from the initiation codon, replacing 7 base pairs with an apparently unrelated stretch of 10 base pairs, reveals a premature stop codon in the N-terminal latency-associated peptide at amino-acid position 140. This mutation is referred to as nt419(de17-ins10). Two base pair substitutions in the second exon, a C→T transition at nucleotide position 610 and a G→T transversion at nucleotide position 676, each yield a premature stop codon in the same N-terminal latency-associated peptide at amino-acid positions 204 and 226 respectively. These mutations are called Q204X and E226X respectively. Finally, a G→A transition at nucleotide position 938 results in the substitution of a cysteine by a tyrosine. This mutation is referred to as C313Y. This cysteine is the fifth of nine highly conserved cysteine residues typical of the members of the TGF-β superfamily and shared in particular by TGF-β1, -β2 and -β3, and inhibin-βA and -βB (McPherron & Lee, 1996). It is thought to be involved in an intramolecular disulfide bridge stabilizing the three-dimensional conformation of the bioactive carboxyterminal peptide. Its substitution is therefore likely to affect the structure and function of the protein. This C313Y has recently also been described by Kambadur et al. (1997).

A conservative phenylalanine to leucine substitution was also found at amino acid position 94 in the first exon, due to a C→A transversion at nucleotide position 282 of the myostatin gene. Given the conservative nature of the amino acid substitution, its location in the less conserved N-terminal latency-associated peptide, and as this mutation was observed at the homozygous condition in animals that were not showing any sign of exceptional muscular development, this mutation probably does not interfere drastically with the myostatic function of the encoded protein, if at all. This mutation is referred to as F94L. The murine protein is characterized by a tyrosine at the corresponding amino acid position.

Also identified was a silent C→T transition at the third position of the 138^{th} cytosine codon in the second exon, referred to as nt414(C-T).

In addition to these DNA sequence polymorphisms detected in the coding region of the myostatin gene, also found were four DNA sequence variants in intronic sequences which are probably neutral polymorphisms and which have been assigned the following symbols: nt374-51(T-C), nt374-50(G-A), nt374-16(del1) in intron 1, and nt748-78(del1) in intron 2 (FIG. 3).

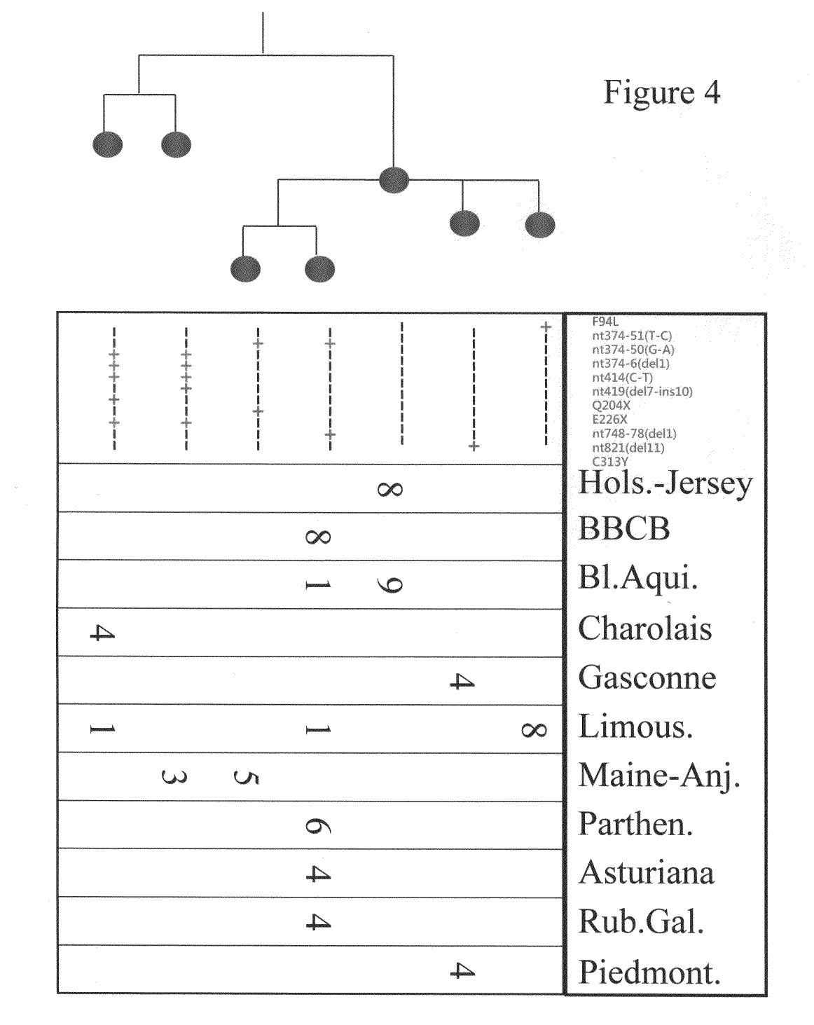

FIG. 4 shows the observed distribution of mutations in the analyzed sample sorted by breed. For the majority of the studied breeds, the analyzed double-muscled animals were homozygous for one of the five described mutations expected to disrupt the myostatin function or compound heterozygotes for two of these mutations. This is compatible with the hypothesis that the double-muscled condition has a recessive mode of inheritance in all these breeds.

Only in Limousin and Blonde d'Aquitaine was there no clear evidence for the role of myostatin loss-of-function mutations in the determinism of the observed muscular hypertrophy. Most Limousin animals were homozygous for the conservative F94L substitution which is unlikely to cause the muscular hypertrophy characterizing these animals, as discussed above. One Limousin animal proved to be heterozygous for this mutation, the other allele being the “wild-type” one. All Blonde d'Aquitaine animals were homozygous “wild-type”. These data indicate either that the myostatin gene is possibly not involved in the double-muscled condition characterizing these two breeds, or that there are additional myostatin mutations outside of the coding region. The double-muscling condition is often considered to be less pronounced in Limousin animals compared to other breeds.

The data indicate that some mutations, such as the nt821del(11) and C313Y, are shared by several breeds which points towards gene migration between the corresponding populations, while others seems to be confined to specific breeds. Moreover, while some breeds (the Belgian Blue breed in particular) seem to be essentially genetically homogeneous others show clear evidence for allelic heterogeneity (e.g. Maine-Anjou).

The observation of allelic heterogeneity contradicts with the classical view that a single mh mutation spread through the European continent in the beginning of the 19^{th} century with the dissemination of the Shorthorn breed from the British Isles (Ménissier, 1982). Two of the mutations at least are shared by more than one breed, indicating some degree of gene migration but definitely not from a single origin.

In mice, and in addition to the in vitro generated myostatin knock-out mice (McPherron and Lee, 1997), the compact mutation can be due to a naturally occurring mutation at the myostatin gene. The compact locus has been mapped to the D1Mit375-D1Mit21 interval on mouse chromosome 1 known to be orthologous to HSA2q31-32 and BTA2q12-22 (Varga et al., 1997).

From an applied point of view, the characterization of a panel of mutations in the myostatin gene associated with double-muscling contributes to the establishment of a diagnostic screening system allowing for marker-assisted selection for or against this condition in cattle.

### Example 1

### Genetic and Physical Mapping

Integration of the HSA2q31-32 and BTA2q12-22 maps were done by using coincident YAC's and the mh locus was positioned in the interval flanked by Col3AI and INPP1 as follows. Genetic mapping was performed using a previously described (Holstein-Friesian×Belgian Blue)×Belgian Blue experimental backcross population counting 108 informative individuals (Charlier et al., 1995). Microsatellite genotyping was performed according to standard procedures (Georges et al., 1995), using the primer sequences reported in Table 1. Linkage analyses were performed with the MLINK, ILINK and LINKMAP programs of the LINKAGE (version 5.1) and FASTLINK (2.3P version, June 1995) packages (Lathrop & Lalouel, 1984; Cottingham et al., 1993). The YAC library was screened by PCR using a three dimensional pooling scheme as described in Libert et al., 1993. The primer pairs corresponding to the CATS used to screen the library are reported in Table 1. Cross-hybridization between SINE-PCR products of individual YACs was performed according to Hunter et al. (1996), using primers reported in Lenstra et al. (1993). Microsatellites were isolated from Yacs according to Cornelis et al. (1992).

### Example 2

### Mapping of the Human Myostatin Gene on the Genebridge-4-Panel

DNA from the Genebridge-4-panel (Walter et al., 1994) was purchased from Research Genetics (Huntsville, Ala.), and genotyped by PCR using standard procedures and the following human myostatin primer pair (5′GGCCCAACTATGGATATATTTG-3′ and 5′-GGTCCTGGGAAGGTTACAGCA-3′, SEQ ID NOS: 9 and 10 respectively). Mapping was performed via the WWW server of the Whitehead Institute/MIT Center for Genome Research using their RH-mapper program (Slonim, D.; Stein, L.; Kruglyak, L.; Lander, E., unpublished) to position the markers with respect to the framework map. Segregation vectors of the query markers were compared with the vectors from all markers in the region of interest in the complete Data Release 11.9 (May 1997) to obtain a more precise position. This positions myostatin in the INPP1-Colb 3AI on the human map with LOD score superior to 3.

### Example 3

### RT-PCR

To clone the bovine myostatin orthologue a strategy based on RT-PCR amplification from skeletal muscle cDNA was chosen. Total RNA was extracted from skeletal muscle (Triceps brachialis) according to Chirgwin et al. (1979). RT-PCR was performed using the Gene-Amp RNA PCR Kit (Perkin-Elmer) and the primers reported in Table 1. The PCR products were purified using QiaQuick PCR Purification kit (Qiagen) and sequenced using Dye terminator Cycle Sequencing Ready Reaction (Perkin-Elmer) and an ABI373 automatic sequencer, using the primers reported in Table 2.

### Example 4

Diagnosis of the nt821(del11) Deletion

To diagnose the nt821(del11) the following primer sequences were designed flanking the nt821(del11) deletion: 5′-TCTAGGAGAGATTTTGGGCTT-3′ (SEQ ID NO:68) and 5-GATGGGTATGAGGATACTTTTGC-3′ (SEQ ID NO:69). These primers amplify a 188 by fragments from normal individuals and a 177bp fragment from double-muscled individuals. Heterozygous individuals show the two amplification products. These amplification products can be detected using a variety of methods. In this example the PCR product was labeled by incorporation of dCTP³², separated on a denaturing acrylamide gel and revealed by autoradiography. Other approaches that could be used to distinguish the three different genotypes are known to those skilled in the art and would include separation in agarose gels and visualization with ethidium bromide, direct sequencing, TaqMan assays, hybridization with allele specific oligonucleotides, reverse dot-blot, RFLP analysis and several others. The specificity of the test is linked to the detected mutation and not to the primers used in the detection method. That means that other primers can easily be designed based on said bovine myostatin sequence that would fulfill the same requirements.

### Example 5

### Determination of Mutations in Other Breeds

A total of 32 animals with extreme muscular development were sampled from ten European beef cattle breeds in which double-muscled animals are known to occur at high to moderate frequency: (i) Belgium: Belgian Blue (4), (ii) France: Blonde d'Aquitaine (5), Charolais (2), Gasconne (2), Limousin (5), Maine-Anjou (4), Parthenaise (3), (iii) Spain: Asturiana (2), Rubia Gallega (2), (iv) Italy: Piedmontese (2). The determination of the double-muscled phenotype of the sampled animals was performed visually by experienced observers. Four animals with conventional phenotype sampled from the Holstein-Friesian (2) and Jersey (2) dairy populations were analyzed as controls.

In order to facilitate the study of the myostatin coding sequence from genomic DNA, the sequences of the exon-intron boundaries of the bovine gene were determined. In mice, the myostatin gene is known to be interrupted by two introns, respectively ≈1.5 and 2.4 Kb long (McPherron & Lee, 1997). Two primer pairs were thus designed, respectively, in bovine exons 1 and 2, and exons 2 and 3, that were predicted to flank the two introns, assuming conservation of gene organization between mouse and cattle (FIG. 3 and Table 3). Using these primer sets, two PCR products respectively 2 Kb and 3.5 Kb long were generated from a YAC clone (179A3) containing the bovine myostatin gene (Grobet et al., 1997). The PCR products were purified using QiaQuick PCR Purification kit (Qiagen) and partially sequenced using Dye terminator Cycle Sequencing Ready Reaction (Perkin-Elmer) and an ABI373 automatic sequencer. Alignment with the bovine cDNA sequence identified the four predicted exon-intron boundaries. The nucleotide sequence corresponding to bovine genomic DNA is identified as SEQ ID NO:54.

TABLE 3
Primers used for PCR amplification and cycle sequencing.

| | | | |
|---|---|---|---|
| Intron1-5′ | 5′-GAAGACGATGACTACCAC | Intron1-3′ | 5′-CTAGTTTATTGTATTGTATCTTA |
| | GCCAGGACG-3′ | | GAGC-3′ |
| Intron2-5′ | 5′-AGACTCCTACAACAGTGT | Intron2-3′ | 5′-ATACTCWAGGCCTAYAGCCTG |
| | TTGT-3′ | | TGGT-3′ |
| Exon1-5′ | 5′-ATTCACTGGTGTGGCAAG | Exon1-3′ | 5′-CCCTCCTCCTTACATACAAGC |
| | TTGTCTCTCAGA-3′ | | CAGCAG-3′ |
| Exon2-5′ | 5′-GTTCATAGATTGATATGGA | Exon2-3′ | 5′-ATAAGCACAGGAAACTGGTAG |
| | GGTGTTCG-3′ | | TTATT-3′ |
| Exon3-5′ | 5′-GAAATGTGACATAAGCAA | Exon3-3′ | 5′-ATACTCWAGGCCTAYAGCCTG |
| | AATGATTAG-3′ | | TGGT-3′ |
| Exon1-Seq1 | 5′-TTGAGGATGTAGTGTTTTC | Exon1-Seq2 | 5′-GCCATAAAAATCCAAATCCTCA |
| | C-3′ | | G-3′ |
| Exon2-Seq1 | 5′-CATTTATAGCTGATCTTCT | Exon2-Seq2 | 5′-TGTCGCAGGAGTCTTGACAGG |
| | AACGCAAG-3′ | | CCTCAG-3′ |
| Exon2-Seq3 | 5′-GTACAAGGTATACTGGAA | | |
| | TCCGATCTC-3′ | | |
| Exon3-Seq1 | 5′-AGCAGGGGCCGGCTGAA | Exon3-Seq2 | 5′-CCCCAGAGGTTCAGCCGGCC |
| | CCTCTGGG-3′ | | CCTGC-3′ |

Reading from left to right and down the table the sequences given in Table 3 are identified as follows: Intron 1-5′ is SEQ ID NO:1(positions 365-391), SEQ ID NO:3(positions 365-391) and SEQ ID NO:54 (positions 772-798); Intron 1-3′ is SEQ ID NO:70; Intron 2-5′ is SEQ ID NO:7l; Intron 2-3′ is SEQ ID NO:72; Exon 1-5′ is SEQ ID NO:54 (positions 324-354); Exon 1-3′ is SEQ ID NO: 73, Exon 2-5′ is SEQ ID NO:54 (positions 2574-2600); Exon 2-3′ is SEQ ID NO:74; Exon 3-5′ is SEQ ID NO:54 (positions 4952-4978); Exon 3-3′is SEQ ID NO:75; Exon 1-seq1 is SEQ ID NO:76; Exon 1-seq2 is SEQ ID NO:1 (positions 209-231), SEQ ID NO:3 (positions 209-231) and SEQ ID NO:54 (positions 616-638); Exon 2-seq1 is SEQ ID NO:77; Exon 2-seq 2 is SEQ ID NO:78; Exon 2-seq3 is SEQ ID NO:1 (positions 594-620), SEQ ID NO:3 (positions 594-620) and SEQ ID NO:54 (positions 2827-2853); Exon 3-seq1 is SEQ ID NO:79; Exon 3-seq2 is SEQ ID NO:1 (positions 1039-1063), SEQ ID NO:3 (positions 1028-1052) and SEQ ID NO:54 (positions 5304-5328).

Based on the available exonic and intronic sequences of the bovine myostatin gene, three primer pairs that jointly allow for convenient amplification of the entire coding sequence from genomic DNA were designed. The position of the corresponding primers is shown in FIG. 3, and the corresponding sequences are reported in Table 3.

After PCR amplification of the entire coding sequence from genomic DNA in the three described fragments, these were purified using QiaQuick PCR purification kit (Qiagen) and sequenced using Dye terminator Cycle Sequencing Ready Reaction (Perkin-Elmer) and an AB1373 automatic sequencer, using the primers used for amplification as well as a series of nested primers (FIG. 3 and Table 3). Chromat files produced with the ABI373 sequencer were analysed with the Polyphred application (D.

Nickerson, personal communication), which is part of a series of sequence analysis programs including Phred (Ewing, B. & Green, P. (1992), unpublished), Phrap (Green, P. (1994), unpublished) and Consed (Gordon, D. (1995), unpublished), but any suitable sequencing program would do, as known to a person skilled in the art.

Monoclonal antibodies (Mab's) specific for myostatin are useful. In the case of the bovine protein having the amino acid sequence identified as SEQ ID NO:2, for example, antibodies can be used for diagnostic purposes such as for determining myostatin protein levels in muscle tissue. To produce these antibodies, purified myostatin is prepared. The myostatin can be produced in bacterial cells as a fusion protein with glutathione-S-transferase using the vector pGEX2 (Pharmacia). This permits purification of the fusion protein by GSH affinity chromatography. In another approach, myostatin is expressed as a fusion protein with the bacterial maltose binding domain. The fusion protein is thus recovered from bacterial extracts by passing the extract over an amylose resin column followed by elution of the fusion protein with maltose. For this fusion construct, the vector pMalC2, commercially available from New England Biolabs, can be used. The preparation of a second fusion protein is also useful in the preliminary screening of MAb's.

The generation of hybridomas expressing monoclonal antibodies recognizing myostatin protein is carried out as follows: BALB/c mice are injected intraperitoneally with protein/adjuvant three times at one-month intervals, followed by a final injection into the tail vein shortly prior to cell fusion. Spleen cells are harvested and fused with NS-1 myeloma cells (American Type Culture Collection, Manassas, Va.) using polyethylene glycol 4000 according to standard protocols (Kennett, 1979; Mirski, 1989). The cell fusion process is carried out as described in more detail below.

The fused cells are plated into 96-well plates with peritoneal exudate cells an irradiated spleen cells from BALB/Cc mice as feeder layers and selection with hypoxanthine, aminopterin, and thymidine (HAT medium) is performed.

An ELISA assay is used as an initial screening procedure. 1-10 μg of purified myostatin (cleaved from the fusion protein) in PBS is used to coat individual wells, and 50-100 μl per well of hybridoma supernatants is incubated. Horseradish peroxidase-conjugated anti-mouse antibodies are used for the colorimetric assay.

Positive hybridomas are cloned by limiting-dilution and grown to large-scale for freezing and antibody production. Various positive hybridomas are selected for usefulness in western blotting and immunohistochemistry, as well as for cross reactivity with myostatin proteins from different species, for example the mouse and human proteins.

Alternatively, active immunization by the generation of an endogenous antibody by direct exposure of the host animal to small amounts of antigen can be carried out. Active immunization involves the injection of minute quantities of antigen (g) which probably will not induce a physiological response and will be degraded rapidly. Antigen will only need to be administered as prime and boost immunizations in much the same manner as techniques used to confer disease resistance (Pell et al., 1997).

Antisense nucleic acids or oligonucleotides (RNA or preferably DNA) can be used to inhibit myostatin production in order to increase muscle mass of an animal. Antisense oligonucleotides, typically 15 to 20 bases long, bind to the sense mRNA or pre mRNA region coding for the protein of interest, which can inhibit translation of the bound mRNA to protein. The cDNA sequence encoding myostatin can thus be used to design a series of oligonucleotides which together span a large portion, or even the entire cDNA sequence. These oligonucleotides can be tested to determine which provides the greatest inhibitory effect on the expression of the protein (Stewart, 1996). The most suitable mRNA target sites include 5′- and 3′-untranslated regions as well as the initiation codon. Other regions might be found to be more or less effective. Alternatively, an antisense nucleic acid or oligonucleotide may bind to myostatin coding or regulatory sequences.

Rather than reducing myostatin activity by inhibiting myostatin gene expression at the nucleic acid level, activity of the myostatin protein may be directly inhibited by binding to an agent, such as, for example, a suitable small molecule or a monoclonal antibody.

It will of course be understood, without the intention of being limited thereby, that a variety of substitutions of amino acids is possible while preserving the structure responsible for myostatin activity of the proteins disclosed herein. Conservative substitutions are described in the patent literature, as for example, in U.S. Pat. Nos. 5,264,558 or 5,487,983. It is thus expected, for example, that interchange among non-polar aliphatic neutral amino acids, glycine, alanine, proline, valine and isoleucine, would be possible. Likewise, substitutions among the polar aliphatic neutral amino acids, serine, threonine, methionine, asparagine and glutamine could possibly be made. Substitutions among the charged acidic amino acids, aspartic acid and glutamic acid, could probably be made, as could substitutions among the charged basic amino acids, lysine and arginine. Substitutions among the aromatic amino acids, including phenylalanine, histidine, tryptophan and tyrosine would also likely be possible. These sorts of substitutions and interchanges are well known to those skilled in the art. Other substitutions might well be possible. Of course, it would also be expected that the greater the percentage of homology, i.e., sequence similarity, of a variant protein with a naturally occurring protein, the greater the retention of metabolic activity. Of course, as protein variants having the activity of myostatin as described herein are intended to be within the scope of this invention, so are nucleic acids encoding such variants.

A further advantage may be obtained through chimeric forms of the protein, as known in the art. A DNA sequence encoding the entire protein, or a portion of the protein, could thus be linked, for example, with a sequence coding for the C-terminal portion of E. coli β-galactosidase to produce a fusion protein. An expression system for human respiratory syncytial virus glycoproteins F and G is described in U.S. Pat. No. 5,288,630 issued Feb. 22, 1994 and references cited therein, for example.

A recombinant expression vector of the invention can be a plasmid, as described above. The recombinant expression vector of the invention further can be a virus, or portion thereof, which allows for expression of a nucleic acid introduced into the viral nucleic acid. For example, replication defective retroviruses, adenoviruses and adeno-associated viruses can be used.

The recombinant expression vectors of the invention can be used to make a transformant host cell including the recombinant expression vector. The term “transformant host cell” is intended to include prokaryotic and eukaryotic cells which have been transformed or transfected with a recombinant expression vector of the invention. The terms “transformed with”, “transfected with”, “transformation” and “transfection” are intended to encompass introduction of nucleic acid (e.g. a vector) into a cell by one of many possible techniques known in the art. Prokaryotic cells can be transformed with nucleic acid by, for example, electroporation or calcium-chloride mediated transformation. Nucleic acid can be introduced into mammalian cells via conventional techniques such as calcium phosphate or calcium chloride coprecipitation, DEAE-dextran-mediated transfection, lipofection, electroporation or microinjection. Suitable methods for transforming and transfecting host cells are known (Sambrook, 1989).

The number of host cells transformed with a recombinant expression vector of the invention by techniques such as those described above will depend upon the type of recombinant expression vector used and the type of transformation technique used. Plasmid vectors introduced into mammalian cells are integrated into host cell DNA at only a low frequency. In order to identify these integrants, a gene that contains a selectable marker (e.g. resistance to antibiotics) is generally introduced into the host cells along with the gene of interest. Preferred selectable markers include those which confer resistance to certain drugs, such as G418 and hygromycin. Selectable markers can be introduced on a separate plasmid from the nucleic acid of interest or, preferably, are introduced on the same plasmid. Host cells transformed with one or more recombinant expression vectors containing a nucleic acid of the invention and a gene for a selectable marker can be identified by selecting for cells using the selectable marker. For example, if the selectable marker encodes a gene conferring neomycin resistance (such as pRc/CMV), transformant cells can be selected with G418. Cells that have incorporated the selectable marker gene will survive, while the other cells die.

Nucleic acids which encode myostatin proteins can be used to generate transgenic animals. A transgenic animal (e.g., a mouse) is an animal having cells that contain a transgene, which transgene is introduced into the animal or an ancestor of the animal at a prenatal, e.g., an embryonic stage. A transgene is a DNA which is integrated into the genome of a cell from which a transgenic animal develops. In one embodiment, a bovine cDNA, comprising the nucleotide sequence shown in SEQ ID NO:1, or an appropriate variant or subsequence thereof, can be used to generate transgenic animals that contain cells which express bovine myostatin. Likewise, variants such as mutant genes (e.g. SEQ ID NO:3) can be used to generate transgenic animals. This could equally well be done with the human myostatin protein and variants thereof. “Knock out” animals, as described above, can also be generated. Methods for generating transgenic animals, particularly animals such as mice, have become conventional in the art are described, for example, in U.S. Pat. Nos. 4,736,866 and 4,870,009. In a preferred embodiment, plasmids containing recombinant molecules of the invention are microinjected into mouse embryos. In particular, the plasmids are microinjected into the male pronuclei of fertilized one-cell mouse eggs; the injected eggs are transferred to pseudo-pregnant foster females; and, the eggs in the foster females are allowed to develop to term. (Hogan,1986). Alternatively, an embryonal stem cell line can be transfected with an expression vector comprising nucleic acid encoding a myostatin protein, and cells containing the nucleic acid can be used to form aggregation chimeras with embryos from a suitable recipient mouse strain. The chimeric embryos can then be implanted into a suitable pseudopregnant female mouse of the appropriate strain and the embryo brought to term. Progeny harboring the transfected DNA in their germ cells can be used to breed uniformly transgenic mice.

Such animals can be used to determine whether a sequence related to an intact myostatin gene retains biological activity of myostatin. Thus, for example, mice in which the murine myostatin gene has been knocked out and containing the nucleic acid sequence identified as SEQ ID NO:1 could be generated along with animals containing the nucleic acid sequence identified as SEQ ID NO:3. The animals could be examined for display of muscular hyperplasia, especially in comparison with knockout mice, which are known to display such. In this way it can be shown that the protein encoded by SEQ ID NO:3 lacks myostatin activity within the context of this invention while the protein encoded by the nucleic acid sequence identified as SEQ ID NO:1 possesses biological activity of myostatin.

In such experiments, muscle cells would be particularly targeted for myostatin (and variants) transgene incorporation by use of tissue specific enhancers operatively linked to the encoding gene. For example, promoters and/or enhancers which direct expression of a gene to which they are operatively linked preferentially in muscle cells can be used to create a transgenic animal which expresses a myostatin protein preferentially in muscle tissue. Transgenic animals that include a copy of a myostatin transgene introduced into the germ line of the animal at an embryonic stage can also be used to examine the effect of increased myostatin expression in various tissues.

The pattern and extent of expression of a recombinant molecule of the invention in a transgenic mouse is facilitated by fusing a reporter gene to the recombinant molecule such that both genes are co-transcribed to form a polycistronic mRNA. The reporter gene can be introduced into the recombinant molecule using conventional methods such as those described in Sambrook et al., (Sambrook, 1989). Efficient expression of both cistrons of the polycistronic mRNA encoding the protein of the invention and the reporter protein can be achieved by inclusion of a known internal translational initiation sequence such as that present in poliovirus mRNA. The reporter gene should be under the control of the regulatory sequence of the recombinant molecule of the invention and the pattern and extent of expression of the gene encoding a protein of the invention can accordingly be determined by assaying for the phenotype of the reporter gene. Preferably the reporter gene codes for a phenotype not displayed by the host cell and the phenotype can be assayed quantitatively. Examples of suitable reporter genes include lacZ (β-galactosidase), neo (neomycin phosphotransferase), CAT (chloramphenicol acetyltransferase) dhfr (dihydrofolate reductase), aphIV (hygromycin phosphotransferase), lux (luciferase), uidA (β-glucuronidase). Preferably, the reporter gene is lacZ which codes for β-galactosidase. β-galactosidase can be assayed using the lactose analogue X-gal (5-bromo-4-chloro-3-indolyl-b-D-galactopyranoside) which is broken down by β-galactosidase to a product that is blue in color (Old).

The present invention includes knocking out wild type myostatin in mammals, in order to obtain the desired effect(s) thereof. This is particularly true in cattle raised for beef production. It may well prove advantageous to substitute a defective gene (e.g. SEQ ID NO:3 or its genomic analogue) rather than delete the entire sequence of DNA encoding for a protein having myostatin activity. A method of producing a transgenic bovine or transgenic bovine embryo is described in U.S. Pat. No. 5,633,076, issued May 27, 1997, for example.

The transgenic animals of the invention can be used to investigate the molecular basis of myostatin action. For example, it is expected that myostatin mutants in which one or more of the conserved cysteine residues has been deleted would have diminished activity in relation to a wild type myostatin protein in which all such residues are retained. Further, deletion of proteolytic cleavage site would likely result in a mutant lacking biological activity of myostatin.

Transgenesis can be used to inactivate myostatin activity. This could be achieved by using conventional transgenesis, i.e. by injection in feritilized oocytes, or by gene targeting methods using totipotent cell lines such as ES (embryonic stem cells) which can then be injected in oocytes and participate in the development of the resulting organisms or whose nucleus can be transferred into unfertilized oocytes, nucleus transfer or cloning.

It is also possible to create a genetically altered animal in which the double-muscling trait is dominant so that the animal would be more useful in cross-breeding. Further, in a particular aspect, the dominant trait would be male specific. In this way, bulls would be double-muscled but cows would not be. In addition, or alternatively, the trait would also be unexpressed until after birth or inducible. If inducible the trait could be induced after birth to avoid the calving difficulties described above.

There are at least three approaches that can be taken to create a dominant “mh” allele. Because functional myostatin, a member of the TGF-8 superfamily, is a dimer, dominant negative myostatin mutations can be created (Herskowitz et al., 1987; Lopez et al., 1992). According to one method, this is accomplished by mutating the proteolytic processing site of myostatin. To enhance the dominant negative effect, the gene can be put under the control of a stronger promoter such as the CMV promoter or that of a myosin gene, which is tissue specific, i.e., expressed only in skeletal muscle. Alternatively, an antisense sequence of that encoding myostatin could be incorporated into the DNA, so that complementary mRNA molecules are generated, as understood by a person skilled in the art. Optionally, a ribozyme could be added to enhance mRNA breakdown. In another approach, cre recombinase generate/ribozyme approach or the Cre-lox P system could be used (Hoess et al., 1982; Gu et al., 1994).

Male Specificity Can be Achieved by Placing the Dominant mh Alleles on the Y Chromosome by Homologous Recombination.

Inducibility can be achieved by choosing promoters with post-natal expression in skeletal muscle or using inducible systems such the Tet-On and Tet-Off (Gossen et al. 1992; Shockett et al. 1996).

Using conventional transgenesis a gene coding for a myostatin antisense is injected, for example, by inverting the orientation of the myostain gene in front of its natural promoter and enhancer sequences. This is followed by injection of a gene coding for an anti-myostain ribozyme, i.e. an RNA that would specifically bind to endogenous myostain mRNA and destroy it via its “ribozyme” activity.

Also, through gene targeting, a conventional knock-out animal can be generated, specific mutations by gene replacement can be engineered. It is possible to inactivate the myostain gene at a specific developmental time, such as after birth to avoid calving difficulties. As mentioned above, this could be achieved using the Cre-lox P systems in which l.ox P sides are engineered around the myostain gene by homologous recombination (gene targeting), and mating these animals with transgenic animals having a Cre transgene (coding for the Cre recombinase existing DNA flanked by loxP sides) under the dependence of a skeletal muscle specific promoter only active after birth. This is done to obtain individuals that would inactivate their myostain gene after birth. As mentioned above, there are also gene targeting systems that allow genes to be turned on and off by feeding an animal with, for example, an antibiotic. In such an instance, one engineers an operator between the promoter of the gene and the gene itself. This operator is the target of a repressor which when binding inactivates the gene (for example, the lac operon in E. coli). The repressor is brought into the cell using conventional transgenesis, for example, by injection of the gene coding for the repressor.

Transgenic animals of the invention can also be used to test substances for the ability to prevent, slow or enhance myostatin action. A transgenic animal can be treated with the substance in parallel with an untreated control trangenic animal.

The antisense nucleic acids and oligonucleotides of the invention are useful for inhibiting expression of nucleic acids (e.g. mRNAs) encoding proteins having myostatin activity.

The isolated nucleic acids and antisense nucleic acids of the invention can be used to construct recombinant expression vectors as described previously. These recombinant expression vectors are then useful for making transformant host cells containing the recombinant expression vectors, for expressing protein encoded by the nucleic acids of the invention, and for isolating proteins of the invention as described previously. The isolated nucleic acids and antisense nucleic acids of the invention can also be used to construct transgenic and knockout animals as described previously.

The isolated proteins of the invention are useful for making antibodies reactive against proteins having myostatin activity, as described previously. Alternatively, the antibodies of the invention can be used to isolate a protein of the invention by standard immunoaffinity techniques. Furthermore, the antibodies of the invention, including bi-specific antibodies are useful for diagnostic purposes.

Molecules which bind to a protein comprising an amino acid sequence shown in SEQ ID NO:2 can also be used in a method for killing a cell which expresses the protein, wherein the cell takes up the molecule, if for some reason this were desirable. Destruction of such cells can be accomplished by labeling the molecule with a substance having toxic or therapeutic activity. The term “substance having toxic or therapeutic activity” as used herein is intended to include molecules whose action can destroy a cell, such as a radioactive isotope, a toxin (e.g. diphtheria toxin or ricin), or a chemotherapeutic drug, as well as cells whose action can destroy a cell, such as a cytotoxic cell. The molecule binding to the myostatin can be directly coupled to a substance having a toxic or therapeutic activity or may be indirectly linked to the substance. In one example, the toxicity of the molecule taken up by the cell is activated by myostatin protein.

The invention also provides a diagnostic kit for identifying cells comprising a molecule which binds to a protein comprising an amino acid sequence shown in SEQ ID NO:2, for example, for incubation with a sample of tumor cells; means for detecting the molecule bound to the protein, unreacted protein or unbound molecule; means for determining the amount of protein in the sample; and means for comparing the amount of protein in the sample with a standard. Preferably, the molecule is a monoclonal antibody. In some embodiments of the invention, the detectability of the molecule which binds to myostatin is activated by said binding (e.g., change in fluorescence spectrum, loss of radioisotopic label). The diagnostic kit can also contain an instruction manual for use of the kit.

The invention further provides a diagnostic kit for identifying cells comprising a nucleotide probe complementary to the sequence, or an oligonucleotide fragment thereof, shown in SEQ ID NO:1, for example, for hybridization with mRNA from a sample of cells, e.g., muscle cells; means for detecting the nucleotide probe bound to mRNA in the sample with a standard. In a particular aspect, the invention is a probe having a nucleic acid molecule sufficiently complementary with a sequence identified as SEQ ID NO:1, or its complement, so as to bind thereto under stringent conditions. “Stringent hybridization conditions” takes on its common meaning to a person skilled in the art here. Appropriate stringency conditions which promote nucleic acid hybridization, for example, 6× sodium chloride/sodium citrate (SSC) at about 45° C. are known to those skilled in the art. The following examples are found in Current Protocols in Molecular Biology, John Wiley & Sons, NY (1989), 6.3.1-6.3.6: For 50 ml of a first suitable hybridization solution, mix together 24 ml formamide, 12 ml 20×SSC, 0.5 ml 2 M Tris-HCl pH 7.6, 0.5 ml 100× Denhardt's solution, 2.5 ml deionized H₂O, 10 ml 50% dextran sulfate, and 0.5 ml 10% SDS. A second suitable hybridization solution can be 1% crystalline BSA (fraction V), 1 mM EDTA, 0.5 M Na₂HPO₄ pH 7.2, 7% SDS. The salt concentration in the wash step can be selected from a low stringency of about 2×SSC at 50° C. to a high stringency of about 0.2×SSC at 50° C. Both of these wash solutions may contain 0.1% SDS. In addition, the temperature in the wash step can be increased from low stringency conditions at room temperature, about 22° C., to high stringency conditions, at about 65° C. The cited reference gives more detail, but appropriate wash stringency depends on degree of homology and length of probe. If homology is 100%, a high temperature (65° C. to 75° C.) may be used. If homology is low, lower wash temperatures must be used. However, if the probe is very short (<100bp), lower temperatures must be used even with 100% homology. In general, one starts washing at low temperatures (37° C. to 40° C.), and raises the temperature by 3-5° C. intervals until background is low enough not to be a major factor in autoradiography. The diagnostic kit can also contain an instruction manual for use of the kit.

The invention also provides a diagnostic kit which can be used to determine the genotype of mammalian genetic material, for example. One kit includes a set of primers used for amplifying the genetic material. A kit can contain a primer including a nucleotide sequence for amplifying a region of the genetic material containing one of the naturally occurring mutations described herein. Such a kit could also include a primer for amplifying the corresponding region of the normal gene that produces functional myostatin. Usually, such a kit would also include another primer upstream or downstream of the region of interest complementary to a coding and/or non-coding portion of the gene. A particular kit includes a primer selected from a non-coding sequence of a myostatin gene. Examples of such primers are provided in Table 3, designated as Exon1-5′, Exon1-3′, Exon2-5′, Exon3-5′ and Exon3-3′. These primers are used to amplify the segment containing the mutation of interest. The actual genotyping is carried out using primers that target specific mutations described herein and that could function as allele-specific oligonucleotides in conventional hybridization, Taqman assays, OLE assays, etc. Alternatively, primers can be designed to permit genotyping by microsequencing.

One kit of primers thus includes first, second and third primers, (a), (b) and (c), respectively. Primer (a) is based on a region containing a myostatin mutation, for example a region of the myostatin gene spanning the nt821del(11) deletion. Primer (b) encodes a region upstream or downstream of the region to be amplified by primer (a) so that genetic material containing the mutation is amplified, by PCR, for example, in the presence of the two primers. Primer (c) is based on the region corresponding to that on which primer (a) is based, but lacking the mutation. Thus, genetic material containing the non-mutated region will be amplified in the presence of primers (b) and (c). Genetic material homozygous for the wild type gene will thus provide amplified products in the presence of primers (b) and (c). Genetic material homozygous for the mutated gene will thus provide amplified products in the presence of primers (a) and (b). Heterozygous genetic material will provide amplified products in both cases.

The invention provides purified proteins having the biological activity of myostatin. The terms “isolated” and “purified” each refer to a protein substantially free of cellular material or culture medium when produced by recombinant DNA techniques, or chemical precursors or other chemicals when chemically synthesized. In certain preferred embodiments, the protein having biological activity of myostatin comprises an amino acid sequence identified as SEQ ID NO:2. Furthermore, proteins having biological activity of myostatin that are encoded by nucleic acids which hybridize under stringent conditions, as discussed above, to a nucleic acid comprising a nucleotide sequence identified as SEQ ID NO:1 or SEQ ID NO:7 are encompassed by the invention. Proteins of the invention having myostatin activity can be obtained by expression in a suitable host cell using techniques known in the art. Suitable host cells include prokaryotic or eukaryotic organisms or cell lines, for example, yeast, E. coil, insect cells and COS 1 cells. The recombinant expression vectors of the invention, described above, can be used to express a protein having myostatin activity in a host cell in order to isolate the protein. The invention provides a method of preparing a purified protein of the invention comprising introducing into a host cell a recombinant nucleic acid encoding the protein, allowing the protein to be expressed in the host cell and isolating and purifying the protein. Preferably, the recombinant nucleic acid is a recombinant expression vector. Proteins can be isolated from a host cell expressing the protein and purified according to standard procedures of the art, including ammonium sulfate precipitation, column chromatography (e.g. ion exchange, gel filtration, affinity chromatography, etc.), electrophoresis, and ultimately, crystallization (see generally, “Enzyme Purification and Related Techniques”, Methods in Enzymology, 22, 233-577 (1971)).

Alternatively, the protein or parts thereof can be prepared by chemical synthesis using techniques well known in the chemistry of proteins such as solid phase synthesis (Merrifield, 1964), or synthesis in homogeneous solution (Houbenwcyl, 1987).

The protein of the invention, or portions thereof, can be used to prepare antibodies specific for the proteins. Antibodies can be prepared which bind to a distinct epitope in an unconserved region of a particular protein. An unconserved region of the protein is one which does not have substantial sequence homology to other proteins, for example other members of the myostatin family or other members of the TGFβ superfamily. Conventional methods can be used to prepare the antibodies. For example, by using a peptide of a myostatin protein, polyclonal antisera or monoclonal antibodies can be made using standard methods. A mammal, (e.g. a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay can be used to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures, thus immortalizing these cells and yielding hybridoma cells. Such techniques are well known in the art. For example, the hybridoma technique originally developed by Kohler and Milstein (Kohler, 1975) as well as other techniques such as the human B-cell hybridoma technique (Kozbor, 1983), the EBV-hybridoma technique to produce human monoclonal antibodies (Cole, 1985), and screening of combinatorial antibody libraries (Huse, 1989). Hybridoma cells can be screened immunochemically for production of antibodies specifically reactive with the peptide, and monoclonal antibodies isolated.

The term antibody as used herein is intended to include fragments thereof which are also specifically reactive with a protein having the biological activity of myostatin, or a peptide fragment thereof. Antibodies can be fragmented using conventional techniques and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments.

It is also known in the art to make chimeric antibody molecules with human constant regions. See, for example, Morrison et al., Takeda et al., Cabilly et al., Boss et al., Tanaguchi et al., Teng et al. (Morrison, 1985; Takeda, 1985; Cabilly; Boss; Tanaguchi; Teng, 1982), European Patent Publication 0173494, United Kingdom Patent GB 2177096B, PCT Publication W092/06193 and EP 0239400. It is expected that such chimeric antibodies would be less immunogenic in a human subject than the corresponding non-chimeric antibody.

Another method of generating specific antibodies, or antibody fragments, reactive against protein having the biological activity of a myostatin protein, or a peptide fragment thereof, is to screen expression libraries encoding immunoglobulin genes, or portions thereof, expressed in bacteria, with peptides produced from the nucleic acid molecules of the present invention. For example, complete Fab fragments, VH regions and FV regions can be expressed in bacteria using phage expression libraries. See for example Ward et al., Huse et al., and McCafferty et al. (Ward, 1989; Huse, 1989; McCafferty, 1990). Screening such libraries with, for example, a myostatin protein can identify immunoglobulin fragments reactive with myostatin. Alternatively, the SCID-hu mouse developed by Genpharm can be used to produce antibodies, or fragments thereof.

The polyclonal, monoclonal or chimeric monoclonal antibodies can be sued to detect the protein of the invention, portions thereof or closely related isoforms in various biological materials, for example they can be used in an ELISA, radioimmunoassay or histochemical tests. Thus, the antibodies can be used to quantify the amount of a myostatin protein of the invention, portions thereof or closely related isoforms in a sample in order to determine the role of myostatin proteins in particular cellular events or pathological states. Using methods described hereinbefore, polyclonal, monoclonal antibodies, or chimeric monoclonal antibodies can be raised to nonconserved regions of myostatin and used to distinguish a particular myostatin from other proteins.

The polyclonal or monoclonal antibodies can be coupled to a detectable substance or reporter system. The term “coupled” is used to mean that the detectable substance is physically linked to the antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride and phycoerythrin; an example of a luminescent material includes luminol; and examples of suitable radioactive material include ¹²⁵I; ¹³¹I, ³⁵S and ³H. In a preferred embodiment, the reporter system allows quantitation of the amount of protein (antigen) present.

Such an antibody-linked reporter system could be used in a method for determining whether a fluid or tissue sample of a subject contains a deficient amount or an excessive amount of the protein. Given a normal threshold concentration of such a protein for a given type of subject, test kits could be thus developed.

The present invention allows the skilled artisan to prepare bi-specific antibodies and tetrameric antibody complexes. Bi-specific antibodies can be prepared by forming hybrid hybridomas (Staerz, 1986 a & b).

Compositions of the invention are administered to subjects in a biologically compatible form suitable for pharmaceutical administration in vivo. By “biologically compatible from suitable for administration in vivo” is meant a form of the composition to be administered in which any toxic effects are outweighed by the therapeutic effects of the composition. The term “subject” is intended to include living organisms in which a desired therapeutic response can be elicited, e.g. mammals. Examples of subjects include cattle, human, dogs, cats, mice, rats and transgenic species thereof. Administration of a therapeutically active amount of the therapeutic compositions of the present invention is defined as an amount effective, at dosages and for periods of time necessary to achieve the desired result. For example, a therapeutically active amount of a compound that inhibits the biological activity of myostatin protein may vary according to factors such as the age, sex, and weight of the individual, as well as target tissue and mode of delivery. Dosage regimes may be adjusted to provide the optimum therapeutic response. For example, several divided doses may be administered daily or the dose may be proportionally reduced as indicated by the exigencies of the therapeutic situation.

In the 2005 publication by Pirottin et al, entitled “Transgenic Engineering of Male-Specific Muscular Hypertrophy” (PNAS, May 3, 2005, vol. 102, no. 18, Pp. 6413-6418) the authors set forth a proof of principle relating to the use of a two-step procedure involving insertional gene targeting and recombinase-mediated cassette exchange in embryonic stem cells specific to transgenic mice. Pirottin et al showed that male mice produced in accordance with the disclosed methodology were characterized by a 5-20% increase in skeletal muscle mass. This led the authors to theorize that such methodology might be applicable to an efficient cattle production system wherein superior beef production and dairy abilities could be realized.

Given that embryonic stem cells are not available for cattle lines, the instant inventors have herein devised an alternative method for producing a genetically modified cattle production system wherein the phenotype exhibits the desirable attributes suggested by Pirottin et al.

By utilizing a somatic cell line, e.g. a fetal fibroblast cell line, in combination with a process for nuclear transfer, the instant inventors have now devised a process which should yield a transgenic line of cattle displaying male-specific muscular hypertrophy.

As illustrated in the following examples, a methodology is suggested for enabling the production of a transgenic bovine line having both male-specific muscular hypertrophy and enhanced dairy production abilities.

### Example 6

### Male-Specific Muscular Hypertrophy

The instant inventors are interested in creating transgenic cattle which produce both milk and meat efficiently.

Intensive breeding programs implemented over the last 50 years have created cattle breeds that are highly specialized in either milk production (e.g. Holstein-Friesian and Jersey) or meat production (e.g. Angus, Hereford, Charolais, Piedmontese, and Belgian Blue). Physiological antagonisms have indeed precluded combining superior abilities for both milk and meat production in the same animal. Despite its effectiveness, the resulting production system can be considered suboptimal because of poor carcass and milk yield of beef and diary cattle, respectively.

The instant inventors envisioned a more efficient alternative based on specialization by sex within the same population: a breed in which cows would be of dairy type and bulls would be of beef type. To achieve this goal the instant inventors proposed to use genetic engineering techniques to target trans-inactivators of myostatin on the Y chromosome. In this way, males are predicted to exhibit muscular hypertrophy akin to “double-muscling”, whereas females will be non-transgenic and fully express their dairy potential.

In order to prove the feasibility of their concept in cattle, the instant inventors generated two transgenic lines of mice in which only the males express a myostatin trans-inactivator in skeletal muscle and consequently show an increase in individual muscles ranging from 5% to 20%.

Applicants note that the experimental design used to produce the described transgenic mice was approved by the ethics committee of the Faculty of Veterinary Medicine, University of Liège.

### Experimental Design/Transgenic Mice

The instant inventors used a two-step procedure involving insertional gene targeting and recombinase-mediated cassette exchange in embryonic stem cells (ES cells) to produce transgenic mice.

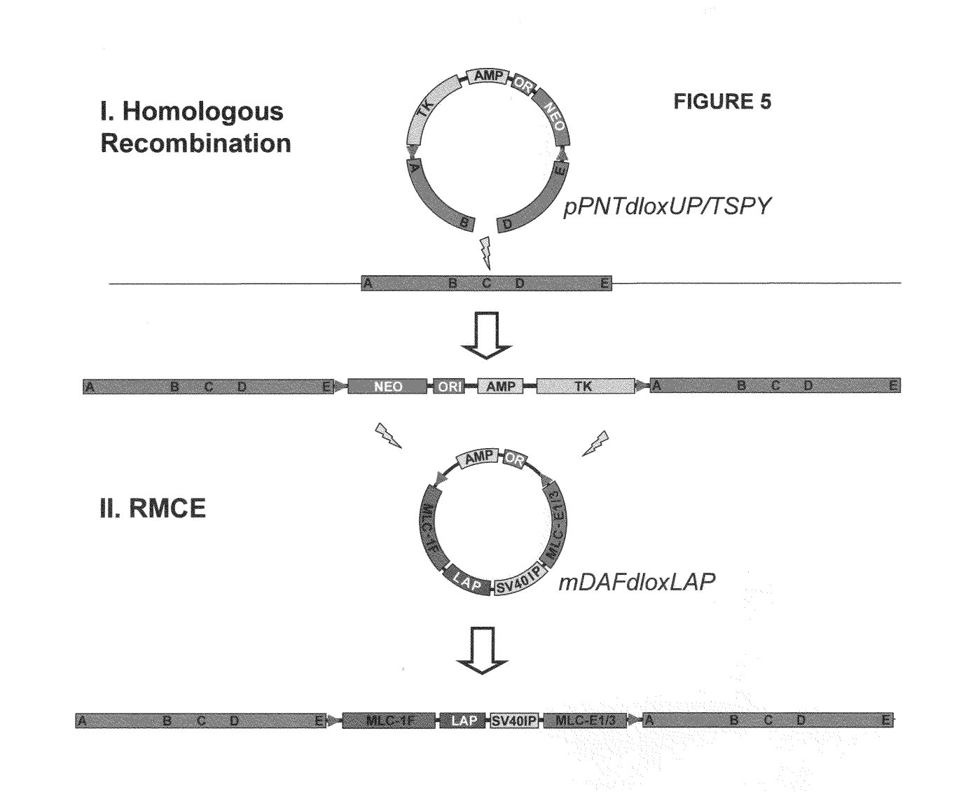

The expression of the murine MSTN “latency-associated peptide” (LAP) or propeptide as a dominant-negative means to repress endogenous myostatin activity (Lee & McPherron PNAS USA 98:9306-9311 2001; Yang et al. Mol. Reprod. Dev. 60:351-361 2001; Thies et al. Growth Factors 18:251-259 2001; Hills et al. Journal of Biological Chemistry 277:40735-40741 2002; Wolfman et al. PNAS USA 100:15842-15846 2003). The testis-specific protein Y-encoded (TSPY) pseudogene was chosen as a targeting site on the murine Y chromosome, since contrary to other mammalian species where TSPY genes are multi-copy, the mouse TSPY is single-copy and non-functional despite being transcribed (Mazeyrat et al. Human Molecular Genetics 7:557-562 1998; Vogel et al. Chromosome Research 6:35-40 1998). As a consequence, the murine TSPy locus is predicted to be non-essential but transcriptionally competent. After Rohozinski et al. (Genesis 32:1-7 2002) the instant inventors chose insertional targeting rather than the usual replacement strategy (which has never been successfully applied to the murine Y chromosome) to insert a cassette containing a positive (neo) and a negative (HSV-tk) selectable marker flanked by heterologous lox sites into the murine Y chromosome. In a second stage, the marker cassette would then be exchanged through cre-mediated recombination with a cassette coding for a myostatin trans-repressor under the dependence of a strong skeletal muscle-specific promoter. FIG. 5 shows a schematic representation of the targeting strategy.

Construction of the Insertional Targeting Vectors pPNYdloxUP and pPNTdloxTSPY

Two adaptors containing (i) a loxP and a SalI site and (ii) a lox2272, a PacI, and a BamHI site were ligated through their shared AflII sticky ends into a 99-bp fragment with XbaI and EcoRI overhangs, which was directionally cloned in the corresponding restriction sites of the pPNT vector to yield the pPNTdlox vector. Homology arms corresponding to nt 31165-39425 [upstream(UP)] and nt 50690-57331 (TSPY) of sequence AC069015 (encompassing the murine TSPY gene) were amplified by using the Expand Long Template PCR system (Roche, Basel, Switzerland) from R1 genomic DNA with primers containing SalI and BamHI sites, respectively, at their extremities. This approach allowed convenient cloning of the PCR products in th pPNTdlox vector to yield the pPNTdloxUP and pPNTdloxTSPY plasmids. Approximately 300-bp gaps were introduced by digestion with Sad (pPNTloxUP) and BbvcI (pPNTdloxTSPY) followed by religation. An adaptor containing unique PmeI and AscI sites was introduced in the Sad site of the pPNTloxdUP. The gapped PPNTdloxUP and pPNTdloxTSPY vectors were completely sequenced before use.

FIG. 5 illustrates this concept. In a first step, an insertional targeting vector comprising a gapped homology arm (A-B/D-E) corresponding to segments of the TSPY locus, heterologous loxP sites (arrows), a positive (Neo) and negative (TK) selectable marker, an ampicillin resistance gene(AMP), and bacterial origin of replication (ORI) is targeted on the Y chromosome by homologous recombination.

Homologous recombination on the murine Y chromosome was successful using these insertional target vectors.

Two distinct insertional targeting vectors were generated by cloning (I) an 8.26-kb homology arm located 13.55 kb upstream of the TSPY pseudogene (pPNTdloxUP) and (ii) a 6.64-kb homology arm spanning the TSPY pseudogene (pPNTdloxTSPY), flanked by heterologous lox sites (loxP and lox 2272; Lee et al. Gene 216:55-65 1998 and Kolb A. F. Analytical Biochemistry 290:260-271 2001), in the pPNT vector providing the neo and HSV-tk cassettes (Tybulewicz et al. Cell 65:1153-1163 1991). The homology arms were obtained by long-template PCR from genomic DNA extracted from R1 ES cells. To enhance targeting efficiency and facilitate screening, 376- and 314-bp gaps (leaving unique AcsI and BbvcI restriction sites for linearization before electroporation) were generated in pPNTdloxUP and pPNYdloxTSPY, respectively. Gene targeting was performed in R1 ES cells by using standard procedures (Nagy et al. PNAS USA 90:8424-8428 1993 and Torres et al. Laboratory Protocols for Conditional Gene Targeting (Oxford University Press, New York) 1997). G418-resistant colonies (677 for pPNTdloxUP and 592 for pPNTloxdTSPY) were screened for successful insertion by using (I) PCR assays based on the use of vector-specific primers combined with gap-specific primers, followed by (ii) Southern blotting with a HSV-tk-specific probe and restriction enzymes cutting in the gap (pPNTdloxUP and pPNTdloxTSPY) and vector (PPNTdloxUP) and (iii) fluorescence in situ hybridization (FISH) by using a pPNT probe and a Y chromosome painting probe. For each construct, one properly targeted clone with euploid karyotype: RI-UP-neotk and RI-TSPY-neotk.

The targeting vectors were linearized with either AscI (pPNTdloxUP) or BbvcI (pPNTdloxTSPY), and 20 μg of resulting products was used to electroporate 10⁷ R1 ES cells with the addition of 25 μg/ml spermidine in the electroporation medium. Positive selection was performed by using G418 (Invitrogen) at 300 μg/ml. After picking and replica plating, colonies having undergone the expected targeting event were identified by performing PCRs with primers located in the gap and selectable markers (neo and HSV-tk). At least two PCRs were performed for each construct, exploring the right and left boundaries of the integration site, respectively. The PCRs were carried out by using the Expand Long Template PCR system. Colonies that appeared positive after PCR screening were further analyzed by Southern blotting. DNA (7.5 μg) was digested with NdeI (pPNTdloxUP) or KpnI (pPNTdloxTSPY) and electrophoresed in a 1% agarose gel before blotting on a nylon membrane by using a standard alkali transfer procedure. The filter was hybridized to a 1,154-bp tk probe excised by BamHI-XbaI digestion from the pcDNA3hsvTK vector (coutesy of F. Princen, University of Liège) according to the manufacturer's instructions (Amersham Pharmacia). Finally, colonies positive by Southern blotting were analyzed by FISH. ES cell metaphase spreads were obtained by following standard procedures (Nagy et al. Manipulating the Mouse Embryo: A Laboratory Manual (Cold Spring Harbor Laboratory Press, Plainview, N.Y.), third edition, 2003). The slides were treated with ribonuclease A and pepsin and fixed with 4% paraformaldehyde. Hybridization was performed at 37° C. in 2×Ssc buffer (1×SSC=0.15M sodium chloride/0.015M sodium citrate, pH 7) containing 50% formamide and 12.5% dextran sulfate. The probes were the flurescein-labeled pPNT plasmid and a Cy3-labeled murine Y chromosome painting probe (Cambio, Cambridge, U.K). The fluorescein signal was amplified by using the Tyramide Signal

Amplification System (NEN/PerkinElmer), and the slides were counterstained with DAPI before microscopic examination.

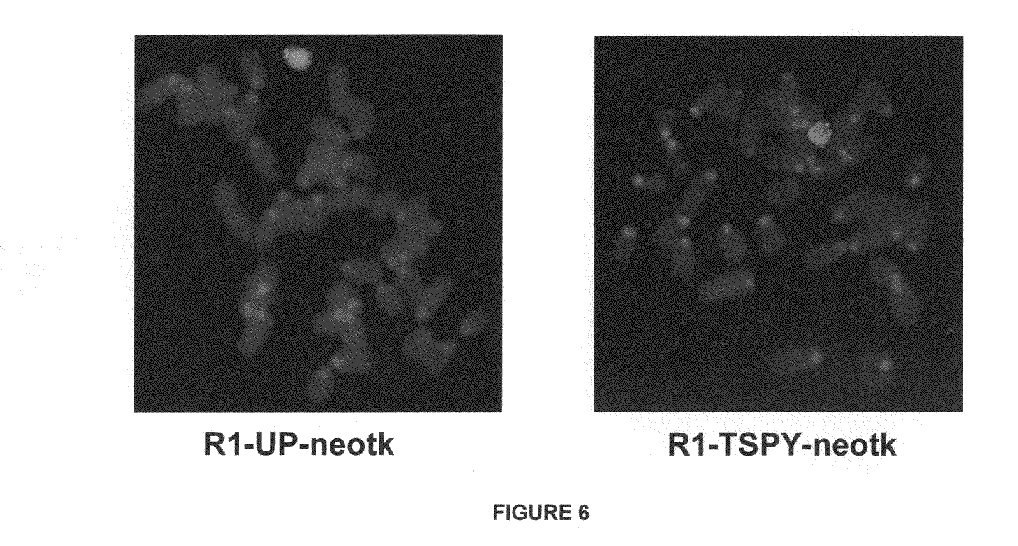

FIG. 6 demonstrates the integration of the transgene on the Y chromosome for both the RI-UP-neotk (left panel) and RI-TSPY-neotk (right panel) clones. ES cell metaphase spreads were hybridized with a fluorescein-labeled transgene-specific pPNT probe (green) and Cy3-labeled murine Y-specific painting probe (red), and counterstained with DAPI.

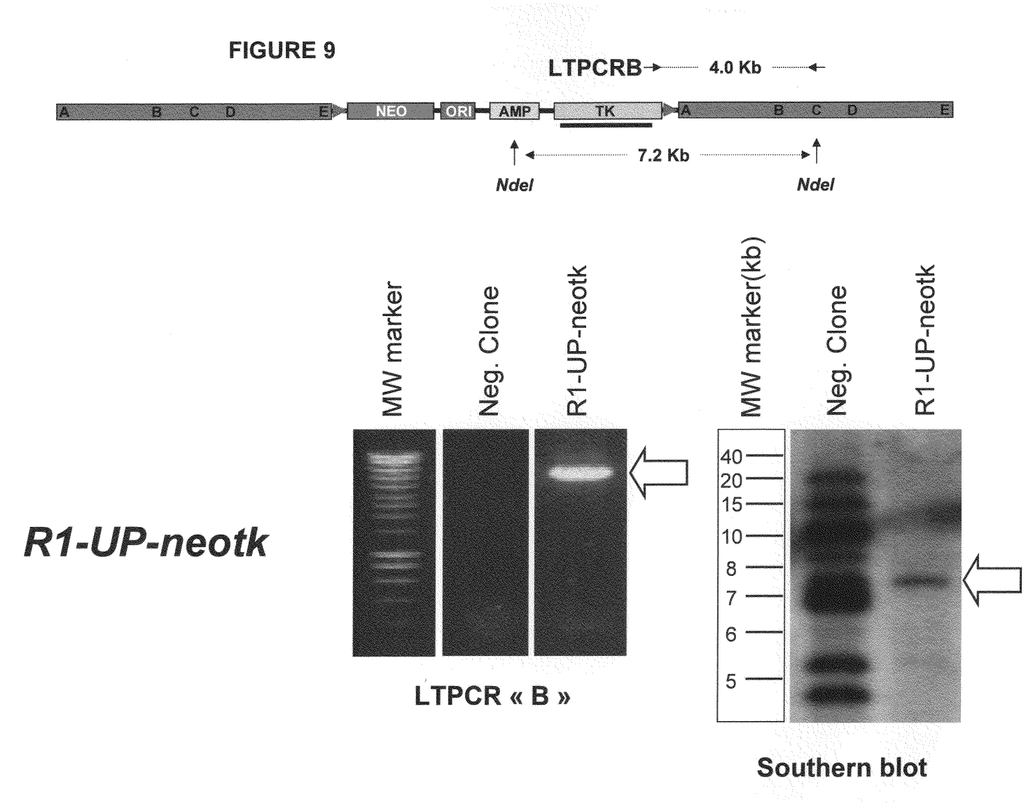

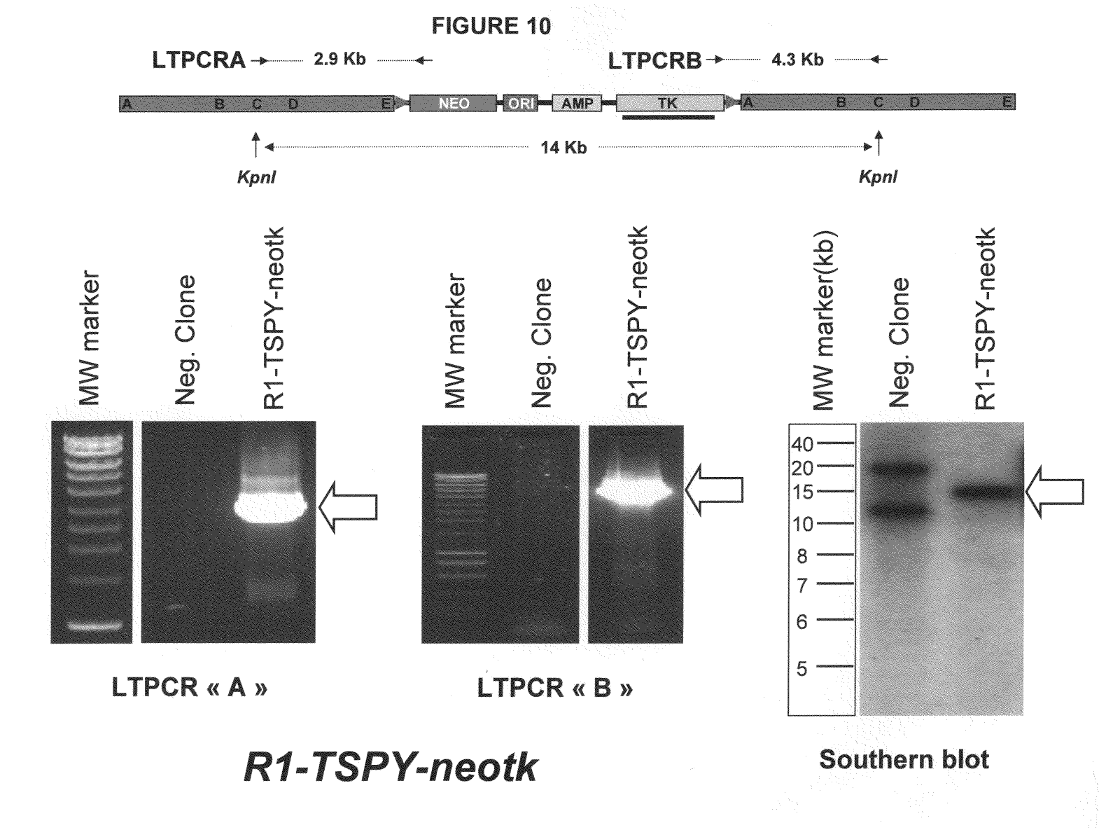

FIGS. 9 and 10 also demonstrate that the ES clones underwent proper gene targeting on the Y chromosome.

The R1-UP-neotk construct is shown in FIG. 9. The position of the primer pairs used for long-template PCR screening (LTPCR), position of the restriction sites, and probe used for Southern blotting are shown. The results of the PCR assay and Southern blotting are shown. The negative clone (Neg. clone) represents a clone that has not undergone the proper gene targeting. The arrows point to the bands of expected size. Most clones that proved positive by PCR appeared to have multiple integrations of the transgene in an autosomal locus, explaining the multiple bands observed for the negative clones by Southern blotting.

The R1-TSPY-neotk construct is shown in FIG. 10. The position of the primer pairs used for long-template PCR screening (LTPCR), position of the restriction sites, and probe used for Southern blotting are shown. The results of the PCR assay and

Southern blotting are shown. The negative clone (Neg. clone) represents a clone that has not undergone the proper gene targeting. The arrows point to the bands of expected size. Most clones that proved positive by PCR appeared to have multiple integrations of the transgene in an autosomal locus, explaining the multiple bands observed for the negative clones by Southern blotting.

Construction of the mDAFdloxLAP Vector

Adaptors containing loxP and lox2272 sites were cloned in the HindIII and EagI restriction sites located, respectively, upstream of the MLC1F promoter (myosin light chain, MLC) and downstream of MLC1/3E enhancer in the mDAF vector (Rosenthal et al. PNAS USA 86:7780-7784 1989). Proper orientation of the lox sites for compatibility with the pPNTdlox vector was verified by sequencing. The MSTN LAP-encoding sequence was obtained by RT-PCR from total RNA extracted from skeletal muscle of 2-month old mice by using TRIzol (Invitrogen). First-strand cDNA synthesis was carried out in a reaction volume of 20 μl starting from 2 μg of total RNA by using an oligo(dT)₁₆as a primer and PowerScript reverse transcriptase (BD Biosciences/Clontech). RT-PCR was performed by using MSTN LAP-specific primers, including either an EcoRI tail or a SmaI tail. The RT-PCR product was digested with EcoRI and SmaI and cloned in the corresponding sites of the mDAFdlox vector. The completed mDAFdloxLAP vector was entirely sequenced before use.

FIG. 5 illustrates this second step of the targeting strategy. The inserted vector sequences are exchanged by RMCE for a cassette coding for the murine MSTN propeptide (LAP) under the dependence of the rat myosin light chain 1F promoter (MLC-1F) and enhancer (MLC-1/3E), appended to the SV40 small tumor antigen intron and polyadenylation signal (SV401P).

### Recombinase-Mediated Cassette Exchange (RMCE)

Three million cells of the RI-UP-neotk and RI-TSPY-neotk ES cell clones were coelectroporated with 25 μg of mDAFdloxLAP and 50 μg of pMCcre plasmid in a buffer containing 25μg/ml spermidine. Gancyclovir-resistant clones (2 μM) were picked and replica-plated. Screening for the expected RMCE event was achieved by PCR with primers located in the UP and TSPY homology arms, the MLC1F promoter (PCR “A”), and MLC1/3E enhancer (PCR “B”). Clones that were positive by PCR were further analyzed by Southern blotting with HindIII restriction enzyme and the MSTN LAP as a probe. The MSTN LAP probe was obtained by PCR amplification of a 850-bp fragment from mDAFdloxLAP.

The MSTN trans-inactivator (LAP) was successfully integrated on the murine Y chromosome by means of RMCE.

The cDNA sequence coding for the murine MSTN LAP was obtained by RT-PCR from total skeletal muscle RNA and cloned into the mDAFdlox plasmid, properly placed under the dependence of the rat MLC1F promoter and 1/3 enhancer for expression in skeletal muscle. The mDAFdlox plasmid corresponds to the mDAF plasmid (Nagy et al. Manipulating the Mouse Embryo: A Laboratory Manual (Cold Spring Harbor Laboratory Press, Plainview, N.Y.), third edition, 2003) in which a 1oxP sequence upstream of the MLC1F promoter and a lox2272 sequence downstream of the MLC1/3 enhancer were inserted. Clones RI-UP-neotk and RI-TSPY-neotk were coelectroporated with the mDAFdloxLAP and pMC-cre plasmids, the later encoding the cre recombinase under the dependence of a tk promoter (Gu et al. Cell 73:1155-1164 1993). Gancyclovir-resistant clones were screened for correct RMCE by (i) PCR assays with UP/TSYP- and MLC-specific primers followed by (ii) Southern blotting with a MSTN LAP probe. Ten RI-UP-LAP 1-10 clones and four RI-TSPY-LAP 1-4 clones having undergone proper RMCE were identified.

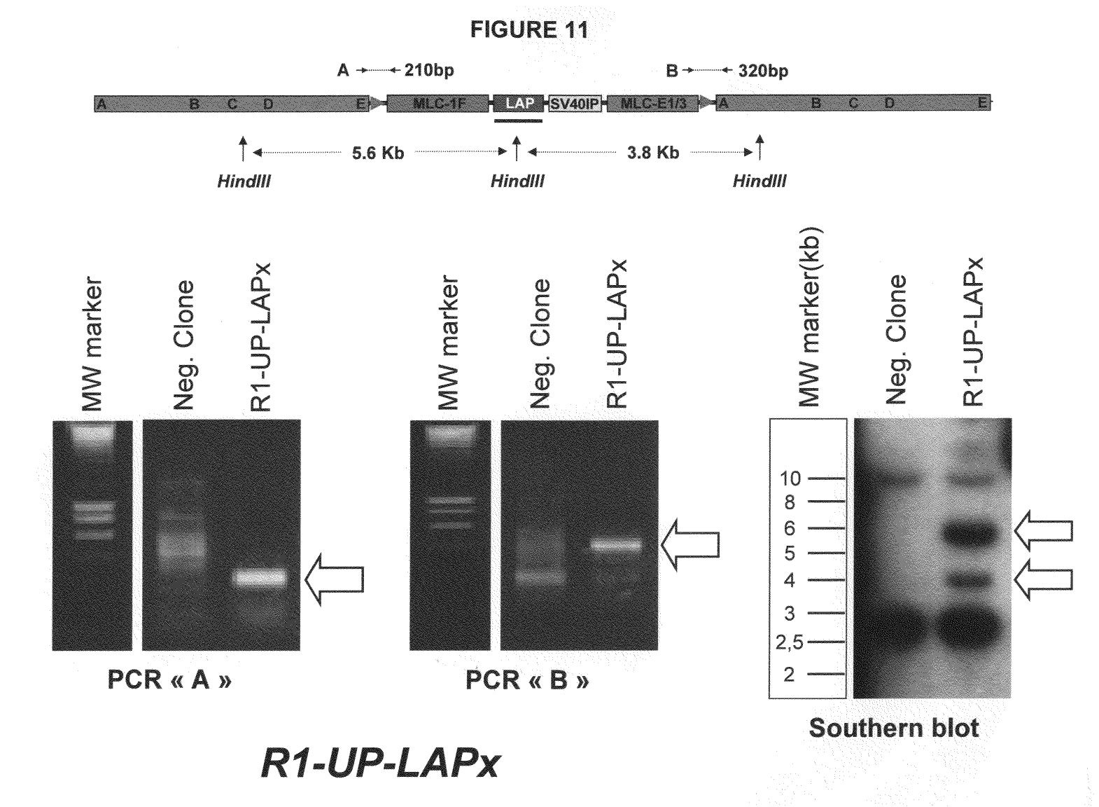

FIG. 11 shows data resulting from the screening for R1-UP-neotk clones having undergone proper recombinase-mediated cassette exchange (RMCE) with the mDAFdloxLAP vector. The position of the primer pairs used for the PCR screening, position of the restriction sites, and probe used for Southern blotting are shown. The results of the PCR assay and Southern blotting are shown. The negative clone (Neg. clone) represents a clone that has not undergone the proper gene targeting. The arrows point to the bands of expected size.

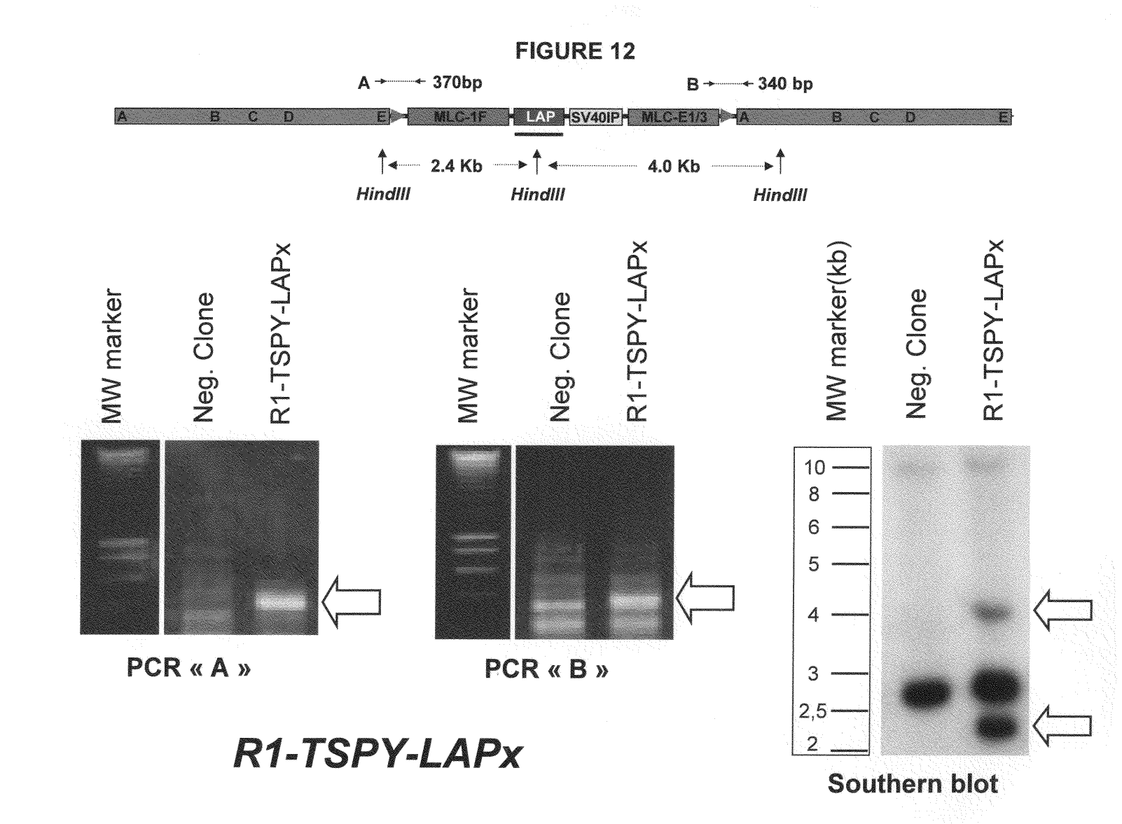

FIG. 12 shows data resulting from the screening for R1-TSPY-neotk clones having undergone proper recombinase-mediated cassette exchange (RMCE) with the mDAFdloxLAP vector. The position of the primer pairs used for the PCR screening, position of the restriction sites, and probe used for Southern blotting are shown. The results of the PCR assay and Southern blotting are shown. The negative clone (Neg. clone) represents a clone that has not undergone the proper gene targeting. The arrows point to the bands of expected size.

### Generation and Identification of Transgenic Mice

C57BL/6J blastocyts (3.5 days old) were harvested and microinjected with targeted ES cells as described in Nagy et al. (Manipulating the Mouse Embryo: A Laboratory Manual (Cold Spring Harbor Laboratory Press, Plainview, N.Y.), third edition, 2003). Uterine transfer was performed the same day in CD1 pseudopregnant mothers by using standard procedures also described in Nagy et al. Individuals carrying the transgene were identified by using a multiplex PCR assay, allowing for the simultaneous amplification of an endogenous MSTN exon 1 fragment (230bp) and a transgene-specific fragment (450 bp) spanning the junction between the MLC1F promoter and MSTN LAP sequence. The ΔMCHR1 allele in the BC-CONT line (CONT, control) was detected by using a multiplex PCR generating a 450- and 700-bp fragment for the knockout and wild-type alleles, respectively.

Four of the R1-UP-LAP clones and all R1-TSPY-LAP clones were used for microinjection into recipient C57BL/6J blastocysts, followed by reimplantation in CD1 foster mothers (Nagy et al.

Manipulating the Mouse Embryo: A Laboratory Manual (Cold Spring Harbor Laboratory Press, Plainveiw, N.Y.), third edition, 2003). Thirteen chimeric males (of which 6 were 100% agouti) and one chimeric females were obtained from R1-UP-LAP clones, whereas a single, 100% agouti chimeric male was obtained from a R1-TSPY-LAP clone. The seven 100% agouti chimeric males were mated to C57BL/6J females to yield an F₁ generation. 110 males and 90 females (P=0.016) were obtained from three of the six R1-UP-LAP-derived males and 69 males and 47 females (P=0.04) were obtained from the unique R1-TSPY-LAP-derived chimera. As expected, all F₁ males were shown by a PCR assay to carry the transgene, whereas none of the females did, thereby confirming the Y-specific integration and germ-line transmission of both UP-LAP and TSPY-LAP transgenes.

### Analysis of Transgene Expression

Total RNA was extracted from muscle and non-muscle tissue by using TRIzol (Invitrogen). Twenty micrograms of total RNA was denatured in formaldehyde load dye (Ambion, Austin, Tex.), electrophoresed on a Reliant Gel System (Cambrex, Rockland, Me.) in NorthernMax Mops gel running buffer (Ambion), and blotted on a positively charged nylon membrane (Amersham Pharmacia) by capillary transfer with 10 mM NaOH in 5×SSC buffer. The membrane was then hybridized overnight with 100 ng of a simian virus 40 (SV40) probe in ULTRAhyb hybridization buffer (Ambion) and washed in 0.1×SSC and 0.1% SDS-containing buffer. The SV40 probe was PCR amplified from the mDAFdloxLAP construct and labeled with ³²P dCTP (Amersham Pharmacia) by random prime labeling (Invitrogen). Membranes were exposed on Hyperfilm (Amersham Pharmacia).

Transgene expression was assayed by Northern blotting with a SV40 probe and total RNA extracted from skeletal muscle, heart, and liver of a 13-week old F₁ male and female from each line. In both lines, transgene-specific transcripts were detected exclusively in male skeletal muscle. As expected, there was no expression of the transgene either in the liver or the heart.

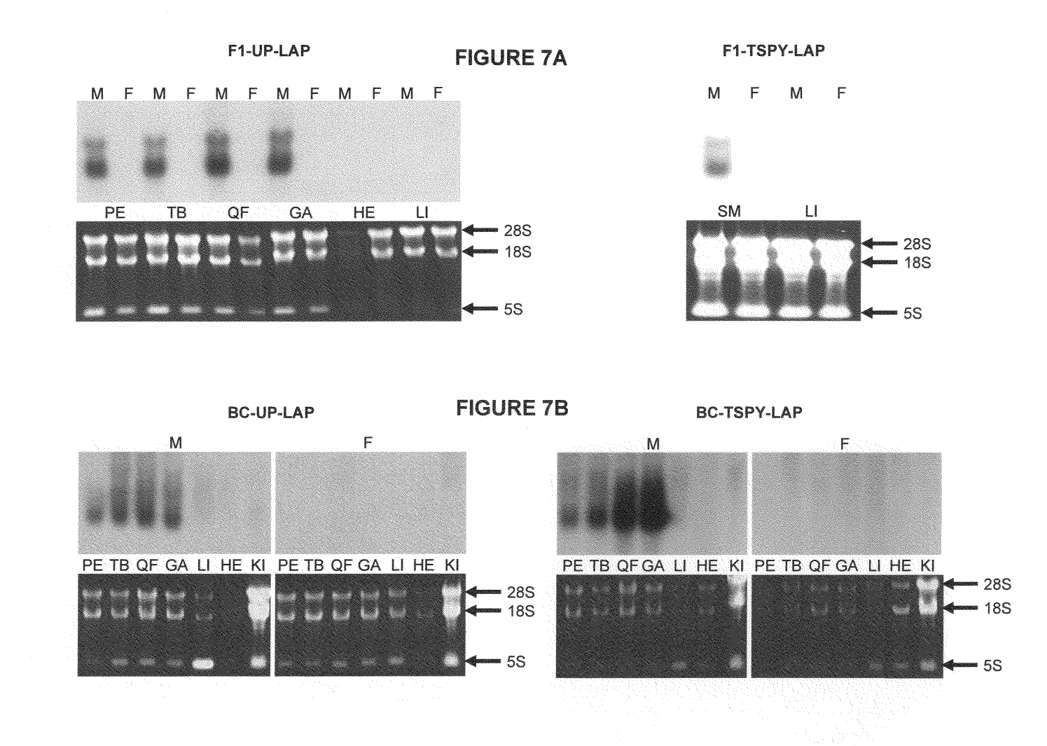

FIG. 7A shows the analysis of this gene expression. Assessment of transgene expression in the F1-UP-LAP and F1-TSPY-LAP transgenic lines by Northern blotting with an SV40 probe and total RNA extracted from pectoralis (PE), triceps brachialis (TB), quadriceps femoris (QF), gastrocnemius (GA), heart (HE), liver (LI) and kidney (KI) is shown in the figure. SM (skeletal muscle) corresponds to mixture of RNA from PE, TB, QF, and GA. “M” and “F” corresponds to samples from males and females, respectively. Ethidium bromide-stained RNA gels before transfer allow for comparison of RNA quantities between lanes. 28S, 18S and 5S correspond to the ribosomal RNAs.

In order to analyze the phenotypic effect of the male-specific MSTN trans-inactivator, four F1-UP-LAP males and two F1-TSPY-LAP males were mated with 129\SV females to produce backcross (BC) animals. To generate a control population (BC-CONT), three non-transgenic males, derived from [(ΔMCHR1)R1>C58BL/6J] chimeric males (Adamantidis et al. European Journal of Neuroscience 21(10):2837-2844 2005) mated to C57BL/6J females, were crossed with 129/SV females. 184 BC-UP-LAPs (87 males and 97 females, P=0.46), 218 BC-UP-LAPs (114 males and 104 females, P=0.50), and 154 BC-CONTs (60 males and 94 females, P=0.006) were produced.

Transgene expression in the BC animals was assayed in skeletal muscle (pectoralis, triceps brachialis, quadriceps femoris, and gastrocnemius), heart, liver, and kidney of a 13-week-old male and female for both BC-TSPY-LAP and BC-UP-LAP lines. As expected, transgene-specified transcripts were detected exclusively in skeletal muscle of males BC-TSPY-LAP and BC-UP-LAP animals. Transgene expression seemed stronger and characterized by an increasing rostro-caudal gradient in the BC-TSPY-LAP animals (FIG. 7B). Such an axial gradient has been reported for a chloramphenicol acetyltransferase transgene drive by the same regulatory elements (Donoghue et al. Development (Cambridge, UK) 116:1101-1112 1992).

FIG. 7B shows the analysis of this gene expression. Assessment of transgene expression in the BC-UP-LAP and BC-TSPY-LAP transgenic lines by Northern blotting with an SV40 probe and total RNA extracted from pectoralis (PE), triceps brachialis (TB), quadriceps femoris (QF), gastrocnemius (GA), heart (HE), liver (LI) and kidney (KI) is shown in the figure. SM (skeletal muscle) corresponds to mixture of RNA from PE, TB, QF, and GA. “M” and “F” corresponds to samples from males and females, respectively. Ethidium bromide-stained RNA gels before transfer allow for comparison of RNA quantities between lanes. 28S, 18S and 5S correspond to the ribosomal RNAs.

BC animals were reared for 10 weeks during which they were weighed weekly. Analyzing the growth curves by using a mixed model including sex, age (weeks 4-10), genotype (UP-LAP, TSPY-LAP, or CONT), sex by genotype interaction, and random individual effects indicated that transgene genotype (both UP-LAP and TSPY-LAP) had a significant (P=0.0004) positive effect on weight; however, the effect did not differ significantly (P=0.96) between males and females. This observation indicates that at least part of the effect on growth is independent of transgene expression, thus probably due to polygenic background effects. R1 ES cells are of 129/SV×129cX/SV geneotype (Threadgill et al. Mammalian Genome 8:390-393 1197), the different BC lines could exhibit phenotypic differences due to variable proportions of 129/SV and 129cX/SV genes.

### Weight Measurements

Live weight was recorded at 4, 5, 6, 7, 8, 9 and 10 weeks of age. Animals were killed at 10 weeks and dissected. The weight of the carcass (skinned body minus head, tail, all internal organs and associated fat and connective tissue), “leg weight” (skinned leg cut at the knee and tarsus level), and weights of the dissected pectoralis, triceps brachialis, and quadriceps femoris muscles were determined.

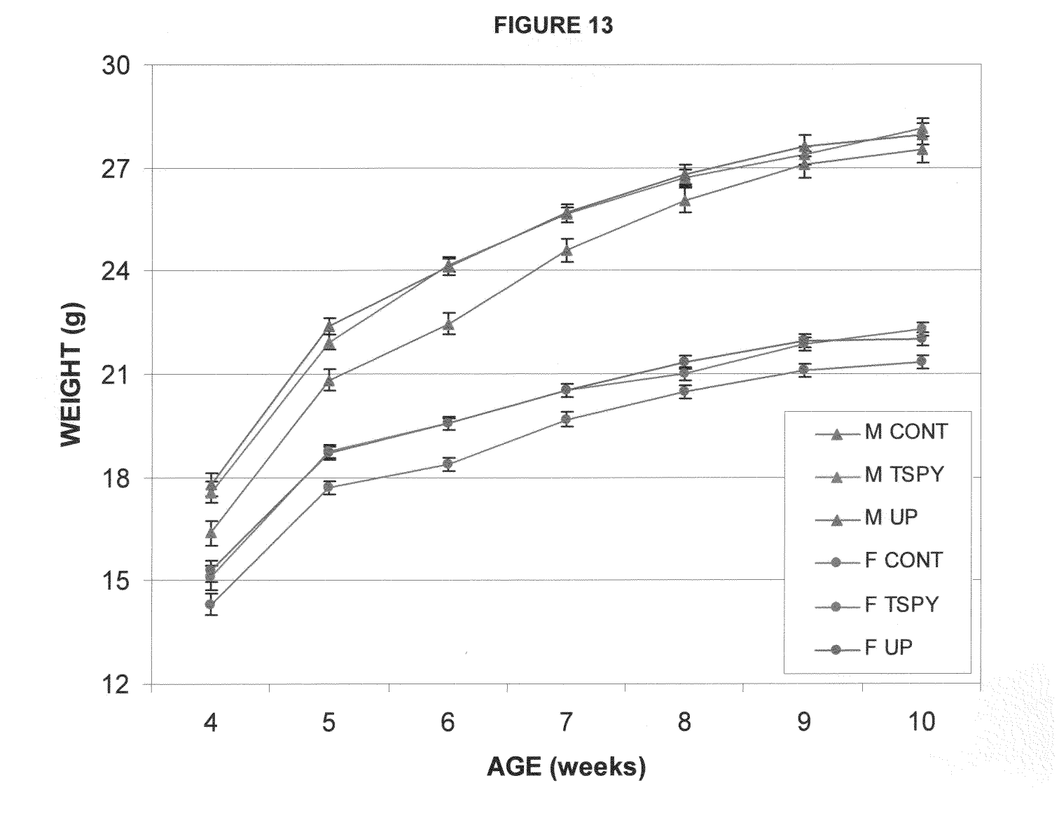

FIG. 13 shows growth curves over 7 weeks (W4-W10) of BC-CONT, BC-UP-LAP, and BC-TSPY-LAP animals sorted by sex (M and F). Error bards correspond to standard errors of the means computed separately for each sex-genotype-week combination.

Growth curves were analyzed with PROC MIXED procedure of the SAS package (SAS Institute, Cary, N.C.). A mixed model including sex, genotype, sex by genotype interaction as fixed effects was used, and a random individual effect accounting for the covariances between repeated measurements (Litell et al. Journal of Animal Science 76:1216-1231 1998) was also used. Relative muscle weights as well as myofiber diameter were analyzed separately for each sex by using the PROC GLM procedure of the SAS package and a model including genotype as a fixed effect.

In order to test for an effect of transgene expression on muscle mass, all BC animals at 10-weeks of age were killed and the carcass, the leg, and a series of individual muscles were weighed. To correct for the differences in live weight observed between lines and individuals, carcass, leg and muscle weights were divided by live weight at slaughter. When analyzing males, both transgenic lines (BC-UP-LAP and BC-TSPY-LAP) exhibited highly significant increases in relative carcass, leg, and individual muscle weights when compared with the control line (BC-CONT). Carcass and leg weights were increased by ≈5%, triceps brachialis weight was increased by ≈10%, and quadriceps femoris weight was increased between ≈15% (BC-UP-LAP) and ≈20% (BC-TSPY-LAP); Tables 4A-B. When comparing females, on the contrary, there was no evidence at all for an effect of genotype (UP-LAP, TSPY-LAP, or CONT) on normalized carcass, leg, or individual muscle weights; Tables 4A-B. These results strongly suggest that the effects observed in the males are caused by the transgenes. The stronger effect in quadriceps femoris (hind legs) when compared with triceps brachialis (front legs) and pectoralis in both lines supports the occurrence of a rostro-caudal gradient and corroborates the Northern blot results in the BC-TSPY-LAP line. Weights of the triceps brachialis and quadriceps femoris were slightly higher in the BC-TSPY-LAP than in the BC-UP-LAP males (P=0.06 and 0.03, respectively), suggesting that the transgenic effect is larger in the former, again corroborating the findings of the Northern blots.

The increase in weight observed for UP-LAP and TSPY-LAP females (FIG. 13) thus likely reflects a proportionate increase in weight of all organs, whereas that of UP-LAP and TSPY-LAP males involves an additional transgene-specific effect on muscle mass.

TABLE 4A
Effect of the transgene on body composition and muscle weight

| | Least square means of body part and muscle weights | | |
|---|---|---|---|
| | relative to live weight ± SE, % (n) | | |
| Body part or muscle | BC-UP-LAP | BC-TSPY-LAP | BC-CONT |

Males

| | | | |
|---|---|---|---|
| Carcass | 41.19 ± 0.27 (24) | 40.57 ± 0.20 (44) | 39.28 ± 0.27 (25) |
| Leg | 1.54 ± 0.02 (24) | 1.51 ± 0.01 (45) | 1.45 ± 0.02 (25) |
| Quadriceps f. | 0.74 ± 0.1 (57) | 0.77 ± 0.01 (67) | 0.64 ± 0.01 (40) |
| Triceps b. | 0.44 ± 0.01 (57) | 0.45 ± 0.01 (68) | 0.40 ± 0.01 (35) |
| Pectoralis | 0.97 ± 0.02 (24) | 0.97 ± 0.01 (45) | 0.94 ± 0.02 (26) |

Females

| | | | |
|---|---|---|---|
| Carcass | 39.07 ± 0.30 (27) | 39.24 ± 0.29 (29) | 38.78 ± 0.31 (25) |
| Leg | 1.47 ± 0.02 (27) | 1.46 ± 0.02 (29) | 1.44 ± 0.02 (27) |
| Quadriceps f. | 0.63 ± 0.01 (66) | 0.64 ± 0.01 (55) | 0.63 ± 0.01 (55) |
| Triceps b. | 0.40 ± 0.01 (65) | 0.39 ± 0.01 (55) | 0.40 ± 0.01 (52) |
| Pectoralis | 0.81 ± 0.01 (27) | 0.80 ± 0.01 (29) | 0.78 ± 0.01 (25) |

TABLE 4B
Effect of the transgene on body composition and muscle weight

| | Statistical significance of genotype effect and | | |
|---|---|---|---|
| | respective contrasts (effect, %) | | |
| Genotype effect | UP-CONT | TSPY-CONT | UP-TSPY |

Males

| | | | |
|---|---|---|---|
| <0.0001 | <0.0001 (4.9) | 0.0002 (3.3) | 0.0703 (1.5) |
| <0.0004 | <0.0001 (6.2) | 0.0023 (4.1) | 0.1482 (2.0) |
| <0.0001 | <0.0001 (15.6) | <0.0001 (20.3) | 0.0266 (−3.9) |
| <0.0001 | <0.0001 (10.0) | <0.0001 (12.5) | 0.0575 (−2.2) |
| 0.4149 | 0.2822 (3.2) | 0.2159 (3.2) | 0.9976 (0.0) |

Females

| | | | |
|---|---|---|---|
| 0.5667 | 0.5148 (0.7) | 0.2905 (1.2) | 0.6852 (−0.4) |
| 0.3305 | 0.1882 (2.1) | 0.2064 (1.4) | 0.939 (0.7) |
| 0.6721 | 0.4791 (0.0) | 0.4064 (1.6) | 0.8731 (−1.6) |
| 0.4572 | 0.4761 (0.0) | 0.2120 (−2.5) | 0.5510 (2.6) |
| 0.2193 | 0.0835 (3.8) | 0.2981 (2.6) | 0.4552 (1.2) |

### Male-Specific Muscular Hypertrophy is Due to an Increase in Myofiber Diameter

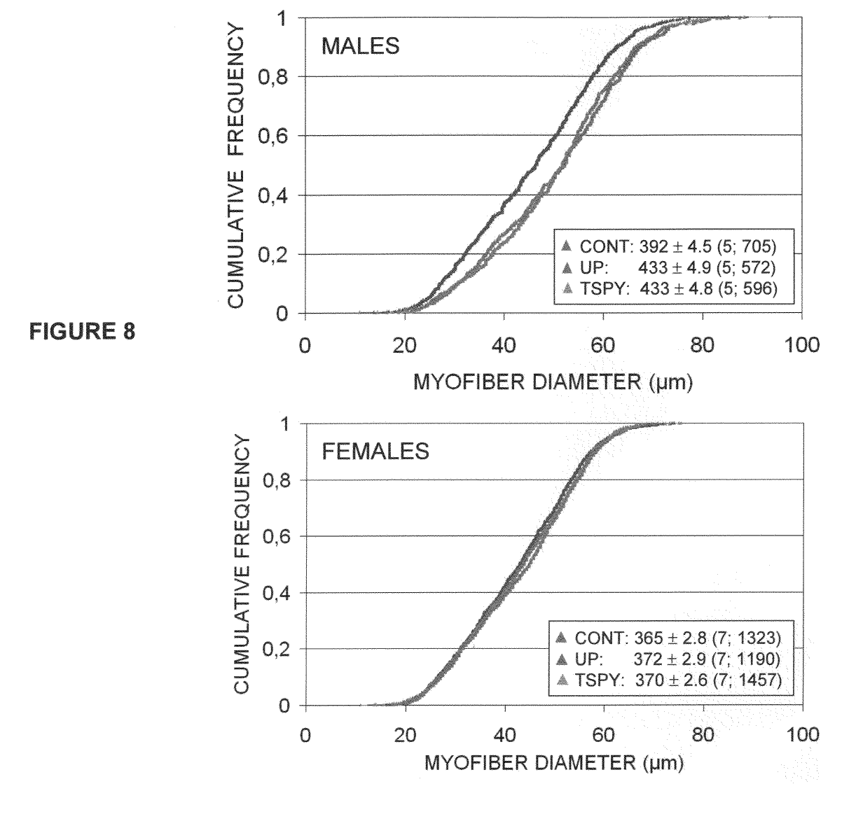

Transgenic expression of the MSTN propeptide has been shown to cause an increase in myofiber diameter (Lee & McPherron PNAS USA 98:9306-9311 2001 and Yang et al. Mol. Reprod. Dev. 60:351-361 2001). To test whether a similar myofiber hypertrophy would have been induced by the transgene in BC-UP-LAP and BC-TSPY-LAP males, histological examinations of transverse sections of the quadriceps femoris was performed. Five 10-week-old males and seven females for each of the three lines (BC-UP-LAP, BC-TSPY-LAP, and BC-CONT) were analyzed. To ensure representativeness, animals with a weight at slaughter within 0.5 g of the mean of their sex-genotype class were selected. The diameter of all myofibers within 10 consistently positioned ×40 microscopic fields for an average of 158 myofibers per individual was determined. FIG. 8 shows the cumulative frequency distribution of myofiber diameter in males and females sorted by genotypes. A highly significant increase in myofiber diameter is seen in both BC-UP-LAP and BC-TSPY-LAP males but not in their female counterparts. Compared with BC-CONT, average myofiber diameter was increased by 10.39% (P<0.0001) and 10.46% (P<0.0001) in BC-UP-LAP and BC-TSPY-LAP males, respectively. Comparable figures were 1.99% (NS) and 1.35% (NS) in females.

### Morphometric Analyses

Ten-week-old mice were killed, and their quadriceps femoris were dissected and fixed in 4% buffered formaldehyde. Muscles were cut transversally at the midpoint and embedded in paraffin. Four-micrometer-wide transverse sections were made from the widest part of the muscle and stained with antibodies against collagen IV to facilitate visualization of individual fibers. Antigen was demasked by pepsin treatment for 60 minutes, and slides were incubated two times (1:5,000 and 1:500) with anti-collagen IV rabbit polyclonal antibody AB748 (Chemicon, Temecula, Calif.). For each muscle section, 10 photographs were taken at ×40 magnification, these photographs being evenly dispersed throughout the section and consistently positioned across individuals. All of the entire myofibers within the microscopic field were measured by using ANALYSIS 3.2 image analysis software (Soft Imaging System, Münster, Germany), and fiber diameter was considered to be the diameter of the largest circle that could be placed within each myofiber.

FIG. 8 shows the cumulative frequency distribution of quadriceps femoris myofiber diameter in males and females of the BC-CONT (blue), BC-UP-LAP (red) and BC-TSPY-LAP (green) lines. Means and standard errors are give for each sex-genotype combination. Numbers in parentheses correspond to the number of analyzed individuals and total number of myofibers.

### Example 7

### Male-Specific Muscular Hypertrophy

### Transgenic Bovine

The production of the transgenic mice described above demonstrates that it is feasible to engineer strains of mammals in which only males express a muscular hypertrophy as a result of the expression of trans-inactivators of the myostatin gene from a transgene integrated on the Y chromosome.

These methods can be optimized for use in cattle.

Currently, embryonic stem cells (ES cells) are available only for application in mice. However, several protocols, for example, nuclear transfer (using somatic cells) useful for successful production of transgenic calves are known in the art (Kuroiwa et al. Nature Genetics 36(7):775-780 2004; Sullivan et al., Biological Reproduction 70:146-153 2004; Kuroiwa et al. Nature Biotechnology 20:889-894 2002; Cibelli et al. Science 280:1256-1258 1998 and U.S. Pat. No. 5,633,076).

The method would involve obtaining a somatic cell, preferably, but not limited to, a fetal fibroblast, introducing a transgene of interest to the somatic cell, introducing the nucleus of the transformed somatic cell to an enucleated oocyte, cultivating the oocyte to obtain an embryo and inserting the embryo into the uterus of a foster mother. The method taught by Kuroiwa et al. 2004, a sequential gene targeting strategy for primary somatic cells, is particularly appropriate.

The TSPY gene used to generate the transgenic mice is not a suitable target site on the bovine Y chromosome as this gene is functional in cattle. Finding alternative target sites, i.e. genes that are transcribed in cattle but have no function, would not present any difficulties for a skilled artisan as a substantial portion of the bovine Y chromosome is currently being sequenced.

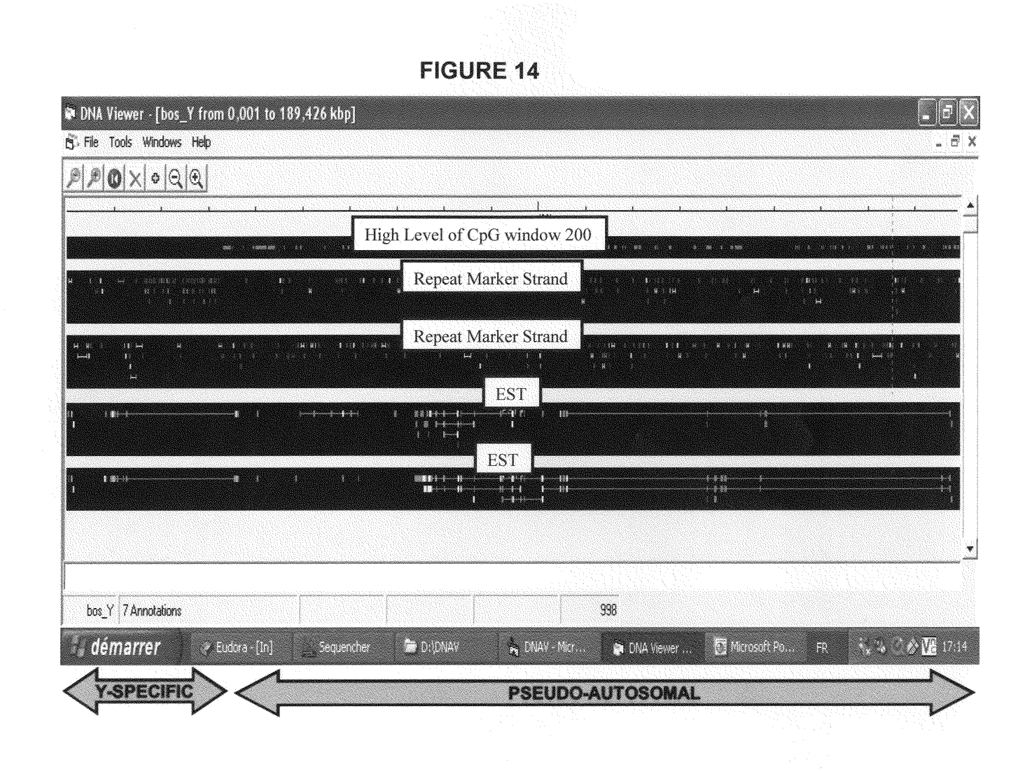

In order to identify suitable target sites on the bovine Y chromosome, the instant inventors have isolated clones from a bovine BAC library containing inserts that originate from the bovine Y chromosome. More specifically, a BAC clone that spans the psuedo-autosomal boundary on the Y chromosome has been sequenced and annotated. The 190 kb insert is identified as SEQ ID NO:80. FIG. 14 shows the position of the CpG islands, repetitive sequence and genes (marked as EST's in the figure). The sequence includes 37 kb of Y-specific sequences, the rest being pseudo-autosomal. The intergenic portions on the Y-specific segment could serve as suitable targeting sites.

Furthermore, additional Y-specific sequences are known and available to the public.

In some cattle breeds, particularly in the BBB, the “double-muscling” phenotype is associated with a high incidence of dystocia, leading to a nearly systematic reliance on cesarean section in some countries. This major drawback has limited the dissemination of the BBB to most countries. The high incidence of dystocia in BBB is due to (i) the extreme muscular hypertrophy characterizing BBB that results from the combined effect of loss-of-function mutation in the myostatin gene and additional “polygenic” effects and (ii) the extreme muscularity of the calf, and also the cow, resulting in a narrowed pelvic channel.

The instant invention remedies this drawback (calving difficulties) because (i) the muscular hypertrophy will be less extreme than, for example, in the BBB, and (ii) the cows will be non-transgenic, and hence, of the dairy type. In addition, one can envisage delaying expression of the myostatin trans-inactivators in order to obtain a postnatal expression of the muscular hypertrophy. Such delayed expression could be achieved by using promoters that are becoming active only in later stages of development or that are inducible through exogenous means. The instant inventors have demonstrated the effectiveness of delayed myostatin invalidation in obtaining late-onset muscular hypertrophy by using cre-loxP-mediated conditional myostatin invalidation (Grobet et al. Genesis 35:227-238 2003).

In summary, the instant inventors, using a two-step procedure involving gene targeting and recombinase-mediated cassette exchange in ES cells, have produced two lines of transgenic mice expressing a dominant-negative latency-associated myostatin propeptide under control of the myosin light chain 1F promoter and 1/3 enhancer from the TSPY locus on the Y chromosome. Males of the corresponding lines are characterized by a 5-20% increase in skeletal muscle mass. This invention enables a more efficient cattle production system combining superior beef production abilities for bulls and diary abilities for cows.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The transgenic animals, oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

### REFERENCES

Particulars of references cited are given below. All of the listed references are incorporated herein by reference.
Barendse, W., S. M. Armitage, L. M. Kossarek, A. Shalom, B. W. Kirkpatrick, A. M. Ryan, D. Clayton, L. Li, H. L. Neibergs, N. Zhang, W. M. Grosse, J. Weiss, P. Creighton, F. McCarthy, M. Ron, A. J. Teale, R. Fries, R. A. McGraw, S. S. Moore, M. Georges, M. Soller, J. E. Womack, and D. J. S. Hetzel. 1994. A genetic linkage map of the bovine genome. Nature Genet. 6: 227-235
Bishop, M. D., S. M. Kappes, J. W. Keele, R. T. Stone, S. L. F. Sunden, G. A. Hawkins, S. Solinas Toldo, R. Fries, M. D. Grosz, J. Yoo, and C. W. Beattie. 1994. A genetic linkage map for cattle. Genetics 136: 619-639.
Boss et al., U.S. Pat. No. 4,816,397.
Cabilly et al. U.S. Pat. No. 4,816,567.
Charlier, C.; Coppieters, W.; Farnir, F.; Grobet, L.; Leroy, P.; Michaux, C.; Mni, M.; Schwers, A.; Vanmanshoven, P.; Hanset, R. & Georges, M. (1995) The mh gene causing double-muscling in cattle maps to bovine chromosome 2. Mammalian Genome 6: 788-792.
Chirgwin, J. M.; Przybyla, A. E.; MacDonald, R. J.; Rutter, W. J. (1979) Isolation of biologically active ribonucleic acid from sources enriched in ribonuclease. Biochemistry 18: 5294-5299.
Cockett, N. E.; Jackson, S. P.; Shay, T. D.; Nielsen, D.; Green, R. D.; Georges, M. (1994). Chromosomal localization of the callipyge gene in sheep (Ovis aries) using bovine DNA markers. Proceedings of the National Academy of Sciences, US, 91: 3019-3023.
Cockett, N. E.; Jackson, S. P.; Shay, T. D.; Farnir, F.; Berghmans, S.; Snowder, G.; Nielsen, D.; Georges, M. (1996). Polar overdominance at the ovine callipyge locus. Science 273: 236-238.
Cole et al. (1985). Monoclonal Antibodies in Cancer Therapy. Allen R. Bliss, Inc. Collins, F. S. 1995. Positional cloning moves from perditional to traditional. Nature Genet. 9: 347-350.
Collina, F. S. 1995. Positional Cloning moves from perditional to traditional. Nature Genetics 9:347-238.
Cornelis, F.; Hashimoto, L.; Loveridge, J.; MacCarthy, A.; Buckle, V.; Julier, C.; Bell, J. (1992). Identification of a CA repeat at the TCRA locus using YACs: a general method for generating highly polymorphic markers at chosen loci. Genomics 13: 820-825.
Cottingham, R. W.; Idury, R. M.; Schäffer, A. A. (1993). Faster sequential genetic linkage computations. Am. J. Hum. Genet. 53: 252-263.
Culley, G. (1807). Observations on livestock. 4th ed., (London,

G. Woodfall).
Fisher, S. R.; Beever, J. E.; Lewin, H. A. (1996). Genetic mapping of COL3A1 to bovine chromosome 2. Mammalian Genome 8:76-77.
Fuji, J.; Otsu, K.; Zorzato, F.; Deleon, S.; Khanna, V. K.; Weiler, J. E. O'Brien, P. J.; MacLennan, D. H. (1991). Identification of a mutation in the porcine ryanodine receptor associated with malignant hyperthermia. Science 253: 448-451.
Georges, M.; Andersson, L. (1996). Livestock genomics comes of age. Genome Research 6: 907-921.
Georges, M.; Nielsen, D.; Mackinnon, M.; Mishra, A.; Okimoto, R.; Pasquino, A. T.; Sargeant, L. S.; Sorensen, A.; Steele, M. R.; Zhao, X.; Womack, J. E.; Hoeschele, I. (1995). Mapping quantitative trait loci controlling milk production by exploiting progeny testing. Genetics 139: 907-920.
Gossen, M. & Bujard, H. (1992). Tight control of gene expression in mammalian cells by tetracycline-responsive promotors. Proceedings of the National Academy of Sciences, USA, 89: 5547-5551.
Grobet, L.; Royo Martin, L. J.; Poncelet, D.; Pirottin, D.; Brouwers, B.; Riquet, J.; Schoeberlein, A.; Dunner, S.; Ménissier, F.; Massabanda, J.; Fries, R.; Hanset, R.; Georges, M. (1997) A deletion in the myostatin gene causes double-muscling in cattle. Nature Genetics 17: 71-74.
Gu, H.; Marth, J. D.; Orban, P. C.; Mossmann, H.; Rajewsky, K. (1994). Deletion of a DNA polymerase beta gene segment in T cells using cell type-specific gene targetting. Science 265: 103-106.
Hanset, R. and Michaux, C. (1985a). On the genetic determinism of muscular hypertrophy in the Belgian White and Blue cattle breed. I. Experimental data. Génét. Sél. Evol. 17: 359-368.
Hanset, R. and Michaux, C. (1985b). On the genetic determinism of muscular hypertrophy in the Belgian White and Blue cattle breed. II. Population data. Génét. Sél. Evol. 17: 369-386.
Hanset, R. (1991). The major gene of muscular hypetrophy in the belgian Blue Cattle Breed. In Breeding for Disease Resistance in Farm Animals, Owen, Axford, eds. C.A.B. International, pp. 467-478.
Herskowitz, I. (1987). Functional inactivation of genes by dominant negative mutations. Nature 329:219-222. Hogan, B. et al., (1986). A Laboratory Manual, Cold Spring Harbor, N.Y., Cold Spring Harbor Laboratory.
Hoess, R. H.; Ziese, M.; Sternberg, N. (1982). P1 site-specific recombination: nucleotide sequence of the recombining sites. Proc. Natl. Acad. Sci. USA 79: 3398-3402.
Houbenwcyl, (1987). Methods of Organic Chemistry, ed. E. Wansch. Vol. 15 I and II. Thieme, Stuttgart.
Hudson et al. (1995) Science 270:1945-1954 with supplementary data from the Whitehead Institute/MIT Center for Genome Research, Human Genetic Mapping Project, data release 11.9 (May 1997).
Hunter, K.; Riba, L.; Schalkwyk, L.; Clark, M.; Resenchuk, S.; Beeghly, A.; Su, J.; Tinkov, F.; Lee, P.; Ramu, E.; Lehrach, H. and Housman, D. (1996). Toward the Construction of Integrated Physical and Genetic Maps of the Mouse Genome Using Interspersed Repetitive Sequence PCR (IRS\NPCR) Genomics. Genome Research 6: 290-299.
Huse et al., (1989). Science 246: 1275-1281.
Kambadur, R.; Sharma, M.; Smith, T. P. L.; Bass, J. J. (1997). Mutations in myostatin (GDF8) in double-muscled Belgian Blue Cattle. Genome Research 7: 910-916.
Kappes, S. M.; Keele, J. W.; Stone, R. T.; McGraw, R. A.; Sonstegard, T. S.; Smith, T. P. L.; Lopez-Corrales, N. L. and Beattie, C. W. (1997). A Second-Generation Linkage Map of the Bovine Genome. Genome Research 7: 235-249.
Kennett, R. (1979). Cell fusion. Methods Enzymol. 58: 345-359.
Kohler and Milstein. (1975). Nature 256: 495-497.
Kozbor et al. (1983). Immunol. Today 4: 72.
Lathrop, M.; Lalouel, J. M. (1984). Easy calculations of lodscores and genetic risk on small computers. American Journal of Human Genetics 36: 460-465.
Lenstra, J. A.; van Boxtel, J. A. F.; Zwaagstra, K. A.; Schwerin, M. (1993). Short interspersed nuclear element (SINE) sequences of the Bovidae. Animal Genetics 24: 33-39.
Libert, F.; Lefort, A.; Okimoto, R.; Georges, M. (1993) Construction of a bovine genomic library of large yeast artificial chromosome clones. Genomics 18: 270-276.
Lopez, A. R.; Cook, J.; Deininger, P. L.; Derynck, R. (1992). Dominant negative mutants of trnasforming growth factor-betal inhibit the secretation of different transforming growth factor beta isoforms. Molecular and Cellular biology 12(4): 1674-1679.
Lyons, A. L.; Laughlin, T. F.; Copeland, N. G.; Jenkins, N. A.; Womack, J. E.; O'Brien, S. J. (1996). Comparative Anchor tagged Sequences for Integrative mapping of Mammalian Genomes. Nature Genetics 15: 47-56.
McPherron, A. C.; Lee, S.-J. (1996). The transforming growth factor β superfamily. In Growth Factors and Cytokines in Health and Disease, Volume 1B, pages 357-393. JAI press Inc.
McPherron, A. C.; Lawler, A. M.; Lee, S.-J. (1997). Regulation of skeletal muscle mass in mice by a new TGFβ superfamily member. Nature 387: 83-90.
Menissier, F. (1982). Present state of knowledge about the genetic determination of muscular hypertrophy or the double muscled trait in cattle. in Current Topics in Veterinary Medicine and Animal Science, vol. 16: Muscle hypertrophy of genetic origin and its use to improve beef production, pp. 387-428. Ed. King and Menissier, Martinus Nijhoff.
Merrifield, (1964]. J. Am. Chem. Assoc. 85: 2149-2154.
McCafferty et al., (1990). Nature 348: 552-554.
Mirski, S. and Cole, S. P. C. (1989). Antigens associated with multidrug resistance in H69AR, a small cell lung cancer cell line. Cancer Res. 49: 5719-5724.
Morrison et al., (1985). Proceedings of the National Academy of Sciences, USA, 81: 6851.
O'Brien, S. J.; Womack, J. E.; Lyons, L. A.; Moore, K. J.; Jenkins, N. A.; Copeland, N. G. (1993). Anchored reference loci for comparative genome mapping in mammals. Nature Genetics 3: 103-112.
Old, R. W. and Primrose, S. B., In: Principles of Gene Manipulation. An Introduction to Genetic Engineering, 4th ed. Oxford University Press. 63-66.
Pell, J. M.; Flint, D. J.; (1997). In: Milk Composition, Production and Biotechnology, Ed. Welch et al., Chapter 19.
Sambrook, J., Fritsch E. F. and Maniatis, T. (1989). Molecular Cloning: A Laboratory Manual. Cold Spring Harbor Lab Press, Cold Spring Harbor, N.Y.
Shockett, P. E.; Schatz, D. G. (1996). Diverse strategies for tetracycline-regulated inducible gene expression. Proceedings of the National Academy of Sciences, USA, 93: 5173-5176.
Solinas-Toldo, S.; Lengauer, C; Fries, R. (1995). Comparative genome map of man and cattle. Genomics 27: 489-496.
Staerz & Bevan (1986a). Proceedings of the National Academy of Sciences, USA, 83: 1453.
Staerz & Bevan (1986b). Immunology Today 7: 241.
Stewart, A. J., Canitrot, Y., Baracchini, E., Dean, N. M., Deeley, R. G., and Cole, S. P. C. (1996). Reduction of Expression of the multidrug resistance protein (MRP) in human tumor cells by antisense phophorothioate oligonucleotides. Biochem. Pharamcol. 51: 461-469.
Takeda et al., (1985). Nature 314: 452.
Tanaguchi et al., European Patent Publication EP171496.
Teng, et al. (1982) Meth. Enzymol. 92: 3-16.
Varga, L.; Szabo, G.; Darvasi, A.; Muller, G.; Sass, M.; Soller, M. (1997). Inheritance and mapping of compact (Cmpt), a new mutation causing hypermuscularity in mice. Genetics, in the press.
Walter, M. A.; Spillett, D. J.; Thomas, P.; Weissenbach, J.; Goodfellow, P. N. (1994). A method for constructing radiation hybrid maps of whole genomes. Nature Genetics 7:22-28. Ward et al., (1989). Nature 341: 544-54

SEQ ID NO: 80

TTATTTTTAAAAACCCTATTATACTTTTTTCTACATTTTTTTTGCCTTTC
CTGTTCTTCTTTTCCCCTGTGGTTAATGTTTAATGCATATAAATCTTTAT
CTACGTCTTTTACTTTTGCGTATCTATTATTTCTTTCTCTTCTTTATTTC
CTTTCTGCTCAACATTTTGTTAATTTTTTTTTTCATTGCTTTATTCCCCA
ATTGGCACCTTGTTCCAGTTTTGCTTTAGTTAGTTTTCTTCTGGTAGATA
TAATTTTGGTTTGCTATGTTAGACAGTTTGATCTATTGTAACTCATTTTA
CTTGGATTCTTTAGATTTTGCTTATGGGTGTATATGTGTATGTGTAAATT
CCATCACACTTTTTATTGTTGCTATAAACTTTGGCCTCTATGTTGGGTTT
TTACAGTTCTGTGGAGTTTTCCTTTTCTTTTCTTTTTTCTTTTTACATCT
GTTATTTTTTCTCTTTATAATTTGAATTAAAATTTTTTTCAACGTATTAT
ATTTTTCTACATTTACCCCTTTGTTTGCCTTTCCCACTCTTCTTTTCCCC
TTGCAATTAACCTTTAATGTATATAAGCCTTCTTTGTCTACCTCTATTTT
ATTTTGCATATCTGTTCTCTCTTTTCTTTCTTTGGGTCCTCTCTACATAT
TTGTACTTTTGTTTTTATTGCTTTATATCCCACTTAGTACATTATGTTAG
TTTTCTTTTCCAGTTTGTGCTATAGTTAGTTTTGTATTTAACTGGTAAAT
ATAATTTTTGATTTACCATCTCCACTCAATCTACTGTAGTTTATTTTTGT
TGGACTCTTTTCACTTTGCTTATGGGTGTATTGGTATATATGTATATTCC
ATTATTTTCATTATTATTTGCCTGATCTTGTAACTGCCATTTGTCTGGAG
TTCATCTTTGGACTCTCATTTTTTGATATTTCCTACCATCTCACTTAATG
CATAACAAACCACTTGTGGAATCTTCAATCATAACCAGAGATCAAGCCCT
GAGAATTGAAGTGAGAGCACTGACTCCAAGATCCTAGACTACAAGAAAAC
TAACCTTCGGGAATATCAAATAGTGAGAACTCACACAAAGGAAACCCTTG
AATACAATACCTGGGATCACCCAGCCACCAGCAGCACCCTATGCTGGACA
CCTCACGCAAAAAACAAGCAAAACAAAAATAAAAACCTAATCAACAGCAA
GCAGGATTACCACATCATTCAGCCTTGCCCATCAGAGGAAATACAAACAA
ACAAAAACTCAGCACGATTCTCATTGGACCAAACTTAGGAGGGCAGAAAC
CAAAAGGAAGAAACWMCTTYWYSCKKGAAGCCTGAGAAAACAAGACCTCA
AACATAGTAAGTTAAAAAAAAATAATGAAAAGGCAGAGAAATACTACACA
AATGAAAGAACAAGCTAGAAACACAGAAGTCCAAATAAATGAAGAGGAAA
TAGGCAAACTACCTGAAAAAGAATTCAGAATAATGATAGTAAAGATGATC
AAAAAACTTGAAAACAAAATGGAGAAAATCTAAGAATCAATGAAGAAAAT
ATAGACGAATTAAAGAATAAACATACTGAGACAAACAACACAATTACTGA
AATTAAAAATATTCTAAAAGGAATCAGTAACAGAATATTTGAAGCAGAAG
ACCAAATCTGTGAGCTGGAAGATAAAATGGTGGAAATAACTGCTGAAGGG
CAGAATAAAGTAAAAAGAATAGAGAATAGCCTCAGAGACCTCAGGGACAA
TATCAAACACACTAACATTCAAATTATAGGGGTCCCAGAATAAGAAAAGA
AGAAATGATACGAGAAGTTTTTTGAAGAGATTATAGTTGAAAATTTCCCT
TAGAAGGAAAAGGAAATAGTCACTCAAGTCCAAGTGTAACAAAGAGCAC
CATACAGGATAAATCCAAGGAGAAATATGGCAAGAAACATAGAAAGCAAA
CTAACAAAGACTAAACAGAAAGAAATATTATTAAAAGCAGCAAAGAAGAA
GTAATGAGTAACATACAAAGGAAATCCCATATGATTAACAGCTTTTCTTT
CAGCAGAAATTCTGCAGGCCCGAGGGGAATGGCAGAATATATGTAAAGTT
CTGAAAGGGAAAAATCTAGCACCAGGATTACAATACCCAGCTAGGATCTC
ATTCAAAATTGAGAAATCAAAAGCTGTTTAGACAAGCAAAAGTTAAGACA
ATTTAGTACCACCAAACCAGCTATACAAGAAATGTTAAAGGGACTTATAT
TGTCAAGAAATACAATAGAATAAAGCAAGTCTACAAAATCAACCCCAAAC
AGTTAAGAAAATAGCTGTTGGAACATATATACCAATAATTATGTTAAATG
GATTAAATGCTCTGACCAAAAGACACAGACTGGCTGAATAGATATAAAAA
CCAGACCCATATAAATATTGTCTACAAGAAACCCACTTCAGACATAGAAT
GCAAGTGAGAGGATGGAAAAATATATTTCATGAAAATGGGAAGAAAAAGA
AAGCTGGAGTAGTAGTCCTTGTATCAGAAAAAATAGACCTGAAACTAAAG
AAGATTATAAGAGATAAGGAAGGACACTACATAATGATCAAAGGATCAAT
CCAAGAGGAAGACATAACAATTGTAAATAACTGTGCACCCAACACAGGAG
CACCTTAATACATAAGACAAACAGTAACAGACACAAAAGGAGAAATTGAT
ACTAACACAAACATAGTGGGAGACTTTAACACCCCACTCATATTGATGGA
AAGATCATCAAAACCGAAAATTAATAAGGAAACACATGTTTTAAATGATA
TATGAGATGAAATGAATCTCATTGATATCTTCAGGGGATTCCATCAAAAT
GCAGAAGAATGTACCTTCTTCTCAAGTGCACATGGAACATTCTCCAGGAT
AGACCACACCTTGGCCACAAATCAAACCTCAGTAAGTTTAAGAAAATTGA
AATTATATCAAGCATCTTCTCAGATCACAATACTATGAAACTAGATATGA
ATTACAAGAAAAACACTGTAAGGAACACGAACAAATGGAGATTAAACCAC
ACTTTTCTAAATAACCAATAGGTTATTGAAGATATCAAAAGCAAAATTTT
TAAAAATCCTTGAAACAAATGACAATGAAAACCTGACAATTCAAAACCTA
TAGTATACATCAAAAGCAATTCTAAGAGGGAAGTTTATAGCAATACAAGC
CTACCTCACAAAACAAGAAAAAGATCGAATAGACAACCTAACTTGACACG
TGAAACAACTGGAAAAAAAAACAAAAAATAAAAAAGGAAAAACAAAAATT
AGTAGAAGGAAAGAAATCATAAAGATTTGAGTAGAATTATAAGAAAATGA
AATAAAAGAGACAATAGTAAAGATTAATAAAACTAAAAGCTGGTTCTTTG
AGATGATAAGCAAAATTGACGACCTTTAGCTAGAGTGAACAAGATAAAAA
GAGAGAAGAATAAAACCAACAAAATTAGAAATGAAAAGGGAGAGGTTACA
ACTGACAATGCAGAAATACAAAGGATTATAAGAGACTATTATGAACAACT
CTATGATAACAAAATGGACAACCTGGGAGAAACAGACAGATTGTTAGAAA
TGCTCAATCTTCCAAGACTGAATGAGGAAGACAGAAATTAGGCACAACCC
AATTACAAGGTCTAAATCAAAGCTGTGATAAAAAATCTCTCAAAAGTCTA
GGAGCAGAAGACTTCATAGGTGAGTTCTATCAAACATTAGAGATGACATA
ATGCTTATCCTTCTGAAACTCTTTCAAGAAACTGCAGAGGAAAGAACACT
TTGAAACTAATTCCACAAGGCCACCATCACCCTGATGCCAAAACCAAAGA
CAACACCAAAAAAGAAAATGACAGGCCAATATCACTGATGAACATAGATG
CAAAAAATCCTCAACAAAATTTTAGCAAACAGAACTCAACAATACATGAA
AAAGCACATACACCATAATCATATTGGGTTTATTTGAGGGATGCTAGCAT
TCTTCAATATATGCAAATTAATCAATATGACAGACCATATTTACCAAATG
AAAGATAAAAATCATATGATAATGATTTTTAATCATTAAAAATCAGAAAT
GCAGAAAAAAAATTAGATTTAAAAATGTAAAATAATTATATTAAAAATGA
AGTTTAAAAAGAAATGAAAAATGGAAAAAAAAAAGTTGAAATGACTCAAA
TGTCCATCAACAAGTGACTGAATAAATAAATCAATGCATTGAAATATTAT
TCAGCCATCAAATTAATGGAGTTCTTATACATTCTATAACATGCTATAGT
TCTGAAATGAYTATGTTGTTTAAAAGAAGCCAGTAACAAAAGCATGCATA
CTTCATTATTCCATGAGTGCATATGTCCCAGATTAGGTAATCCTCTAAAG
ACAGAAAATAGAGGCATAGATACCAGGGCMCAGATGAAGTGGAAAATAGG
AYATSYCTGCCAACTGGACCATGTTTCCCACTGGAGTGATGAAAATATTC
TAGAATGAGGCAGTCATTATGGTTATGCAACCTATGAAGAAATTACAAAT
CAATGAATTATACAGTTWAACTGGGAAATTCATGGTAGATGAACAATATG
TCAATAAATAAAAAAAATTTTTTTTGATTACTGACCTTAACTTCTACGTC
CAGGAATATTATCCTTCAAAAATAAAGRAGRAGTAGATATTTTCGTATGA
ACAAAAAATTMSSRSAWWWAKKTTGAGCTGATGTCTGGTCTGTTGGAAAT
GTAAAAAGGCATTATTTACTGGATAAAATGATATTGAAGAGAAACTATGA
GTTAAGAGTGTCATGTTGAAGTTTGCTACAAAACAAGTAAATTCTAGAAA
GCAATTATCCTTAAATGAATAAATAAATTGAAAAAATAAATAAAGGCAAA
TTGAAATGAAGGATTATTCTTATTATTAATTGGTAAAAGATAAATAATGG
GCTTCTCCATCTTTACTTTTCACTGATCATGAATCTTCATAGTTAAAGTG
CATTTCATATATACAAGATATATTTTGACTCAGAAGTAACGAATCTGCCT
GCAATACAGGAGCCATAGCAGACACGAATTCCATCTTTGTGCCAGGAAGA
TCCCCTGCAGAAGAGCATGGCAACCCACTCCAGTATTCTCGCCTGGAGAA
TTCCCAAGGCAAGAGGAGACTGGTGTGCTTACAGTCCATGGGGTGGCAAA
GAGTTGGACACGACTGAGCAACTAAACAACAGACACAGATTCCTGACAGA
AACTCTGAAGATACTAGAAATACATGAGAAAGTTCTCAGCCTAAACAAAG
CACAAAAACCCTAGACTGTAGAGCTGAATTATAAAAAACCTAGACACAAG
AGTTGAATTACAAAAGATTTTAGGCAATAAAATGTCTCCCAACAGTACTG
TAAACAGTGTGTTCCACACAAGGGAGAGGAGAAATTTTTAATACTCATTC
AATAGAACAACTTTTCTAGTTACATTCATAAACTAATGAAGTAAAAATGT
CCAGAAAATAAACATAGTATAAAATCTGTTGATCATGTTATATCTAATTA
AGACTAAAATTGTAAGGGAATTTTAAGGAAAAAAAATGTGCAAAAGATAC
AGTTAAAGCATCCACAATGAATGTAATTTTATTTTGTTTTTTCCATGGTC
TCAAAATAACTGAAAAATAAGAATGTATTTCATTTTAAAATATATTTTTC
AAATTCAGCAGTATATTTTATTTTCAAATATAAGCAGATTTTGTACTTTT
CAAGCTAAAAATGTTTGTACCTTGCAGTGAATACTTTTTGTTTCACTGAT
TCAGAGGTAAATAAAAGCACGTTATGTGTCTTTACCTTGACAATTTTTGT
GGTATCACTGTGTATTTAGAATCAATGGTTTAAACATGGAGTGTGTAATA
CTACATTCCTTCAGGGACTGTAGGCAATGTAGGAAGTGGTCAAGGCCTTC
CAAACCATTAAGTATGCTCTGTGGAATGGAGTAGTGGTAACCATGGCAGC
CTGCGTTGGCAACAACTGTTTAACAAGGTTTGTCTTAGTTAACTCCCTCA
TAGAAGACAATGAATTTTTAAAGATAAGTACTAAATCTGTGGCCATATAA
AATCATATATATATGATTATACATGCATACAATATGTGTGTGCATATGTG
TATGCTGCTAAGTTGCTTCAGTTGTGTCCGACTCTGTGCGATCCCATAGA
TGGCAGCCCACCAGGCTCCACCATCCCTGGGATTCTCCAGGCAAGATCAC
TGGAGTGGGTTGCCATTTCCTTCTCCAATGCATGAAAGTGAAAAGTGAAA
GTGAAGTCACTCAGTTGTGTCGGACTCTTAGTGACCCCATGGACTGCAGC
CGTCCAGGCTTCTCCATCCATGGGATTTTCCAGGCAAGAGTACTGGAGTG
GAGTACCATTGCTTTCTCTGGCATATGTGTGTATATATATATATGTATAC
TAAAATTTTGTTCCTTGTGAATAATAAGTAAAACATTAAAACCTGACATA
AATGACAAAAACTTTTTAAAACTTAAGCCAACAAAATTTGAATTTGCAGG
AGAAACATATTTAATTACTGACATTATTTTAGAATACTGGAGTATGGAGA
TTGTAATAAAAACAGAACCAGCTTTTACTTGATTTTATTCTTGTAATAAA
ACTCCTCGTGGGCAATGCCTCCCAGTGATGCCAGCACTGGATGTTGGCCA
CTCCCCCTGACCCACCTGTGGAAGGCCACTGCTCCATTCAAAATTCCTTA
GCTATAAAGGGCCTAATCAGTATTGCTTTTGTGACCTACAAAAACCGCAA
TTATTTTCCTTCCTTTCTTTCCTCCTACCTCTCCCTTTTCTTTCCTTTTC
TTTCTTATTATACAATTCATAAAGCTTGCTTCCTGTTTATAGTTATTACA
AAATACTGGCAGTATTCCCCATGTTCTACAATACATTCTTGAGCTCATCT
TACACCTAACACCACCTCTGTGTTGCCCCTTCTCCTCCCCACTATCTGCT
GGTAACCAGTTGATGTTCTTTGTATCTGTTATATTCTCTACTTTGTGGTA
TTTTAAGATTCTATAAGTGATATCATAAAGTATTTGTCTTTCTTATTTTA
CTTGTTGCTGCAAAAGGCATTATTCCATTCTTTTTATGGCTGAGTAACAT
TTCATAGGTCTTTCTTGGTGGCTCAGTGGCAAAGAATCTGCCTGTCAAGA
AGGAGATGTGAGTTAAAAACCTAGGTAAGGTGTATCCCTTGGAAAAGGAA
ATGGCAACCCACTAGAGTATTCTTGCCTGGAAAATTCCACAGGCAGAGGA
GCCTGGAGTGGGTAGAGTCCATAGGGTGGCCATGAGTGGGAAATGACTTA
GTGACTGAACAACAATGAGTGTCCCATAGTACATACATACTACACCTTTA
TACATTCATCCATCATCTATTGGACATTTAGATTGCTTCCATGTCTTAGC
GAATGGTATGCTTCAAGGAACATTGGGATACATGTCTTTTTAATTAATGT
TATGGGTATTCTTTTAATATATATATCCAATAATGAAACTGTTAGGTCAT
GTGTTCCATTCTTAGCTTTTTGAGAAATCTTCATACTAAAAACCATCATT
ATTTCATTCCTATGGACTATCTTTTAGTAAGCAAGTTTGAGTTTCTGAGG
CTGTTAATTGCTGACATCACACCTTGAGGTGTAATAAACACTACTATGAG
GTAGATGCCCTAGGGGCAACTACTTTGTATGCCTTTTAGCAAATGGCACT
GCCACCCCACACATGACATGGCTCCCCAGCTAACTTCCCTCCTCTGCTTT
CACACACAGAAACACCGTGTCCTCTTCCTCTTACATTTCTTCAAAGAAAT
GTATCTTTTGGGCTCAGTCTGAACAGAGTTCTGTCCCATACTTTCTGGTT
AACTGGACCTTCTAGTCTTGTCTCCAATACCACAGGACCTATCTCCTTCA
CTCTTCACACACTTATCCCTTCCCTGCCTCTCTGCTCCACACCATGTTTC
AGAGAGTTCATCAGAGCTCTCATCAGAGAGCTCATTTCACATCAGGTTGA
TTTTATTTAATTTTCATTATTAATATATGCTGTTGGTAGAAGATTCAATG
TCTGTAGTGTTGGCCATTTCTCAGTGGCGAGACACCCGAAGAATTCTGAG
ATCTCTAGCCATCTTCTCAAGTCACTAGCTGCCCATATGTCCAGCTTGAA
TGGCAGGGCTCTCCATGATCTCTGGGGCCTCTTCCAGTCCGTTCTGATTC
AGCTCTTCATTCACCTTGGAACACTGGCTACAGAGCTTTGACCCTCTAAT
TTTCTTAAGTTTCTCTGTGGTGGAGGCTTTCTGAGGAGCTTTCTTGGTCA
GCTTTGCTTGAATATGCTTATTGACCCCCATGGCCACCTGGAGGGTAAAA
AGAGGGAGAACAAAGAATGGCATTGTTTTGGGAAAAAGGGCAATGATGGT
GGTATTGATGGCTTCAAGAGAAGGGATGTGGGCCAAGAAGGGGCATGTGC
AAGCCAAGGACAAGATAGGGTGTGTATGAGGTGGTGTCAATGGGCAAGTG
GAGATTGGGCGGGTTCACTCACCTAGACATTTCTTTGGACTTCTGTTGAT
AGGAGGTCTTCATCTTTTCATCCTGGATATAAGGAGTTGGCTGTTCCATA
ACTGTGCTAGAGCCTTTTAGCTTCCAAGTTACCTTAGTCATTTTGACGTC
TCCCAGTGGTCTGCTCCAGGGCCCTTGAGCACTCCTATATAGGGGAGACC
ACACCCTTATGATCATGAGGTCAGGAAGTCACTTCCTCAGCCAGTGGCGG
CCCTGGGCAAGTCTTGACATCAAAAAGCCTCATCCTGATACCTCTAAGGG
AAGATTAGGAGCAACTCAGATGATTTACTGGGAGAAATGAAGCATGTTTA
ATGCTCCTAGAGAGCCCTCCAAACTCACCTGCCATCTTCATCTCTCTAAG
ACAACACAGATAGGTCACATGGTAGAGAGTAAGTGTCCTCCAAACCTTTC
CTTACAGCCAGTTTTCAGCCTTTCATTCTGTTCTCTCTCATGTTTAACTG
TTATCCAGATTTTATTATCTTGCCTAAAATCCTTCCATGGTAATTAAGCA
GTGGAAATGAGAATCATTTCACTTTTCTCCTACAAGTGGTCTTAAAGCAT
CTTGAACACCATTAAATTCTGAACCACCTACCAGAAACCTTTTCCATCCC
ACCCCCTTTTCTAAGTGTTCGCATACCTTTCCTCGATTTTCTAGTTTCCA
GTCTGAAACCCCTGTCTTCGGTCTGTGAGACTGCACTGACTACACTTGCT
CTTGTTCCAATTGGAGCTGTCTTGTTTCAAATTTAGGACTCATGTGCCGG
AGTCCAACTCTAGCAACCACAGATTCAACTTGAAAAGGTGAACAGTGTTG
GCCAATGAGACAGCCTCTCAGTTTTCTATGGACTGCCTGTTTATTTCAAG
TTTAAGATTATCTTTTACACTTTTACAAAAACATTGGGCCAGAAGTTTGA
CATTTTCAGTTCCCCATCTCCCAGATATATTATCTCCATAAATCATTGTC
GCTCTTCAAACAGAGTTCCTGCTTCAGTGATTCTCTCAGAATCAGCCCTC
CTCTAATGTGTCCTATAGTTAACTTATGATTACATTGTAACTCATGCTAC
ATTCCTCAGTTTACTACTTATCTTCCTAAATCCTGTCTGCCCTTGATATC
TCGAGCTCACTATCTCTTGAAAAAGACTTCTAGCTATAGTGTCTAAAAAT
CCCTAACTTCTATAAACTATAGTAAAATATGCTAACTTTACAACATTCCT
TAAATCTTTAACTTCTAACTATTTTAATTATTTCTGAGCCCTAAATTCAG
TAAATTCCTTTGCCATAAACATTTTCCTCACAAATAGACTTCAGATATCA
ATCCCTCCCATGGCCTCAAGCTACGGCCTATGTGCTCATCCTGGAACACT
CTTTTGTAAAAGTCCTTGAACAAATGTCAATGATTAACTTTATGAATTAT
TTTCTGAGCACAGCTGCAGAAGGCTTTGTGCGTTCTCATGCTCCTCTCGA
GAACAATAAGCACCTTAATATTCCTTTTCAGTCAACTCAGCCGACGAGAG
GAAAAGACAAGTCAGAATTACAAGGCCTAACTCCTTCATCCCAGGATCCA
TGCCTGCAGAATGAGGAGAGGGGTCTGAGGGCCGTGCCTCCATTTTGTTA
GTAATGCCTAAGGCAGCTCCTGACACTCATGGTCACCTCACTCGGTATAA
TACCCAGGCTTCGGTTTCAACAGGTTGGTGGGGATGAGGCAGAGAAATGC
ATGCTTCAGGTTGCACTGAATGATGCCAAGAAGGAAATGACCACCTGTGA
CTGATCAGTATTCCCAGGAAGAGGTAGCACTTGCCCAGTGGTATGTGAGC
CTCATGTGCAAAGGTGTTCTCAACATGTGCCCTCACAAATCTCTTTTCTT
ATCCTGTGTTCCTGCCAGCACCTCCTTTGGAATTTACCTCACAGTCGAGA
CCATGGTGGAATTCATTTTCATGAATAGCCACCACAAGCTTCCAGCTTCT
TGGAGGAAACACTGAGTAATCATGTCCTGCCCATCTAGTTCCCAAGATCT
CTCATTGAGGTTGTTGGCACTGGAGGATACGGGAATTACTGGGGTCCAGC
CCCGGTTGGATCCACGTATTCCTTGGGAGGATGGCGTTGGCAAAATGATA
GATAAAAGGAGAAAAAGACAGAGGCTTGAACTAACTGGTTTACGCAGAAA
GCCAATAAAACCTGTGACATCAGGTTTGCACTGACCACGCAGGCCACAGG
TGCCCTCTCAAATCGTAGAAGGTGCCCACTTTAGGCACCTTCTCGAGTGG
GTCTTAGAAGCCTGGGCAAATAAGTGGTCTCAGAGGTCCCCCACACTCCA
AATTAGTCWTCCTGAAGGAAGAACAGAGAAGAAAAGGAGAGAAAAGGAAA
CAAGAAARAACGACACAGCGAGACCTAGCTTGATGAGCATGGCCTGCAAC
TTTATTTTCCAAAGTAGCTTTTATACCTTAAGTTGTGCATAGAGGATAAT
AGGGGGTGTAGAGTCATGCAAGGTCGGCAGTCCTTGACTCTTATCGAAGC
CAGGCTTTCTTTCTGCAAACTTATCACDTGCAAAGGCTTTAGGTGATTTA
CATCATCTTCTGGCCAGGAGGCCTGTTAACATTTTATGACCCTTTCTTCT
GAATAATGGTTAGTCAATCAGAAAACTTATTTTCTCTAAAGGTGATTATT
CTAAAGTCAGGCGCCACCCTCTGAAAGCATTAGATAAAGTTGCATTCCTA
TAGGGCAAAAGTGTGGTGGGTTATAACAAGAAAAGAATTAACTCAAGGGT
CCAAGGTTACAAACATTAAAGCTACTACTTACATTTCTATATACCAACTA
TCTTAATCAATACACACCCAGGGACACAGTAGTTAAGGGATATGGAAACT
TGGCAGCACGCATTAGCTCAACAAAGAGATCCTCTACTAGTTCTATTCTA
ACAATTTTAACTCTCTGAGAAGCTCTGCATTGTTAGAATATCTTAAGCTT
CCCGTGCCTCTTGTGGTTGGGAGGCTGTGAACAATCACATGCATACCTGC
AGGAGTCCAAACAAACCTGTCAGGCAAGCTTGAAAGTCATCAGAGGGGTT
TGAATTGAAACACTCCTATTATGCCCAGGAGACTTATTAACTAGAGCCCT
AAGTTGATTTTCTTCAGAGAAAGGTGGTCGGGGATAGCCCCCCATTAATG
TCAGAAGAGTTGGTGAAAGTCGTGAAATAGTAAAACAGACAGATTTTGGT
TTTGGGGTAGATGGTTGGGCATATCCAGGGGGCCTCTAGAGTTCTGATTC
ACCTTTGCATGTCAGATCCTCTCTGCWTGACCTTTGTCATGGGTGGGAAC
TCCTGTGCTGGCTTCCAGCAGGGAATGAGTTGTGTGCTTCACTGCAGTCC
TTGTATTCCTAATTGTCCAGCAGGGTCAGCCAGCCTGAGATGTGCCCCTG
GGGGACACTGTTCATTGGCTTAGTGGGACTTCCTGTGATTTCTGTGAAGT
GCTCACTTCGTGGTGTCCACTGGTTGCTTATTCACCCAGGACACATCACA
CTGCTCAAAATTTTAAGAGGTAGAACAGCACACCATAAACAAACCGAAAA
GATGACTCAATATTTTTATAACAAAGAACCTAAAACCTGATAGAAAAACG
TGAAAAGACATTGGCCAACAGTTTCTAAGAAAACTGTAACACTGACCTTT
AACTTTATTAAGAGATGTTTAAAAGACATGAGAGGCAATTGGCAAGAAAG
GTATGGAACTAGTCGCTAACCACATTAAAAAGATATGTAATCTCACTAAG
GTAAGAAATATACTTGGATGCAAGCTCAAAAATGACACAATGATCTCTGT
TCATTTCCAAGGCAAAGCATTCAATATCACAGTTTTCTAAGTCTATGCCA
TGACCAGTAATGCTGAAGAAACTGACAATGAATGGTACTATGAAGACCTA
TAAGACCATTTAGAACTAACACCCAAAAAAAGATATTCGTTTCATTATAG
GGGATTGGAATGCAAAAGCAAGAAGTCAAGAAATACCTGGAGTAAGAGGC
AAATTTGACCTTGGAGTACAGAATGAAGCAGGGCAAAGGCTAGATAGCAT
ATTGAAAAGTGGAAACAATTTCTTTTGTGGGAGCTCTCAATGACCTTTTA
TATATTCCATGGACACAGAGTCTGCCTAGCTGATTATGTGGATTTAATCT
GCAACTTTTATGCTGGTAGGAAGGTTTGCATTTTCTTTTTTAGCCACACT
GACCACAGTGGGTTTCCATTGTGGTTTTATTTCCATCTCCCCATATGATT
CATTCACTGGGGTTTGCTCCTGAGGCTGCCCTGCAGGAATTGGGTTTGCC
CCTGGGACATGTGGAGAGAATAACCTTTGCCCTGCTTCATTCTGTACTCC
CAAATGCTGCTGCTGCTTGGGTCACAGGCATTTTGGCAGCACCAGGTACT
CAGGACGGTAGGCACTGTAGAGAGGGCATCAGAGGGCATCAGAGGGCAGA
CGGACTGAAAACACAATCACAGGAAACTAGCCAGTCTGATCAGAGAAGGC
AATGGCACCCCACTCCAGTACTCTCACCTGCAGAATCCCATTGAGGGAGG
AGCCAGGTGGGCTGCAGTCCATGGGGTCGAGAAAATGAGACATGACCAAG
CGACTTCATTTTCACTTTTCACTTTCAAGCATTGGAGAAGAAAGTGGCAA
CCCACTCCAGTGTTCTTGCCTGGAGAATCCCAGGGACGGGGGAGCCTGGT
GGGCTGCCGTCTATGGGGTCGCACAGAGTCGGACATGACTGAAGTGACTT
AGCAGCAGCAGCAGCAGCCGGTCTGAAAACATGGACCACAGCCTTGTCTA
ACTCAATGAAACTAAGTCATGCCTTGTGGAGCCACCCAAGATGGAGAGGT
CATGGTGGAGAGGTCTGACAGAATGTGGTCCACTGCAGAAGGGAATAGCA
AACAACTTCAATGTTCTTGCCTTGAGCTCTGTTCGTTTCCAAGGCAAGCC
ATTCAATATCACGGTAATCCAAGTCTATGCCCCAACCAGTAACACTGAAG
AAGCTGAAGTTGAACAGTTCTATGAAGACCTACAAGACTGTCTAGAACTA
ACACCCAAAAAAGAAATTCCTTTCATTATAGGGGACTAGAATGCAAAAGT
AGAAAGTCAAGGAACACCTGGAATAACAGGAAAATTTGGCCTTGGAGTAC
AGAATGAAGCAGGGAGAAAGCTAATAGACTTCTGTCAAGAGAACACACTA
ATCATAGCAAACACCCCCTTCCAACAACACAAGAGAGACTCTACACATGG
ACATCACCAGTGGTCAACACCACAATCAGATTGGAGAAGTTCTATACAGT
CAGCCAAAACAAACAAGACTGGGAGCTGACTGTTGCTTAGATCATGAACT
CCTTATTGCCAAATTCATACTGAAATTGAAGAAAGTGGAAAAACCACTAG
ACTATTCACGTATGACCTAAATCAAATCCCTTAGGACTATACAGTGGAAA
TGAAAAAAGGATTTAAGAAACTAGATCTGATAGACAGAGTGCCGGATGAA
CTATGGATGGAGGTTTGTGACATTGTACAGGAGAAAGGAATCAAGACCAT
CCCAAAGAAAAAAATGCAAAAAAAGCAAAATGTCTGTCTGAGGAGGCCTT
ACAAATACCTGTGAAAAGAAGGGAAGTGAAAAGCAAAAAGAAAAGGAAAT
ATACATCCATTTGAGCGCAGAGTTCCAAAGAATAGCAAGGAGAGATAAGA
AAGCCTTTCTCAGTGATCAATACAAAGAAATAGAGGAAAACAATAGAATG
GGAAAGATTGGGGATCTCACCAAGAAAATTAGAGATACAAAGAGAACATT
TCATGCAAAGATGGTCTCAATAAAGGACAGAAATTATATGGACCTAACAG
AAGCAGAAGATATTAAGAAGAGGAGGCAAGAATACACATAATTGTGCAAA
AAAGATATTCACAACCCAGATAATCACAATGGTGTGATCACTCACCTAGA
GCCAGATATCCTTGAATGTGAAGTCAAGTGGCCCTTAGGAAGCATCACTA
TGAACAAAGCTAGTGGAGGTTATGGGATTTCAGTTGAGCTATTTCAAATC
CTCAAAGATGATGCTGTGAAAATGATGCACTCATATACGCCAGGTGCACT
CAACACGCCAGGAAATTTGGAAAACTCAGCAGTGGCCACAGGACTTGAAA
AGGTCAGTTTTCATTCCAACACCAAAGAAAGGCAATGCCAAAGAATGCCC
AGACTAGTGCACAATTGCACTCATCTCACATGCTAGTAAAGTAATGTTCA
AAATTCTCCAAGCCAGGCTTCAGCAATATGTGAACTGTGAACTTCCAGAT
GTTGAAGCTGGTTTTAGAAAAGGCAGAGGAACCAGAGATCAAATTGTCAA
CATCCACTGGATCATGGAAAAAGCAAGAGAGTTCCAGAAAAACATCTATT
TCTGCTTTATTGACTATGCAAAAGACTTTGACTATGGATCACAATAAACT
GTGGACAATTCTGAAAGAGATGGGAATACCAGACCACCTGACCTGTGTCT
TGAGAAACCTGTATGCAGGTCAGGAAGCAACAGTTAGAACTGGACATGGA
ACAAGAGACTGGTTCCAAATAGGAAAAGGAGTATGTCAAGGCTGTATATT
GTCACCCTGCTTATTTAACTTCTATGAAGAGTACATCATGAGAAATGCTG
GGCTGGAAGAAGCACAAGCTGGAATCAAGATTGCAGGAAAAATATCAATA
ATCTCAAATATGTCGATGACACCACCCTTATGGCAGAAAGCGAAGAAGAA
AAGAGCCTCTTGATGAAAGTGAAAGAGGACAGTGGAATATTTGGTTTAAA
GCTCAACATTGAGAAAACTATGATCATGGCATCCGGTCCCATCACTTTCA
TGGTAAATAGATGGGGAAACAGTGGAAACAGTGGCCAACTTTATTTTGGG
GAGCTCCAAAATCACTGCAGATGGTGACTGAAGCCATGAAGTTAAAAGAC
GCTTACTCCTTGGAAGGAAAGTTATGGCCAACTTGGACAGCATATTAAAA
AGCAGAGACATTACTTTTTCAACAAAGGTCCATCTAGTCAAAGTTTTGGT
TTTTCCAGTAGTCATGTATGGATGTGAGAGTTGGACTCTAAAGAAAGCTG
AGTGCCAAAGAATTCATGCTTTTGAACTGCAGTGTTGGATAAGATTCTTG
AGAGTCCCTGGGCTGCAAGGAGATCCAACAAGTCCATCCTAAAGCAGATC
AGTCCAAGGTGTTCATTGGAAGGACTGATGTTAAAGCTCAAACTCCTCAC
ATGAAGAGCTGACTCATTGGAAAAGACCCTGATGCTGGGAAAAATTGGAG
GCAGGAGTAGAAGTGGACGACAGAGGATGAAATGTGTGGATAGCATCACC
GACTCAATGGACATGGGTTTGGGTAGACTCCAGCAGTTGGTGATGGACAG
GGAGGCCTGGCATGCTGTAATTCATGGGGTCACAAAGAGTCAGACTCAAC
TGTGCAACTGAACTGAACAGGCTAGTCTGTCATTTGACTGAGCCAATGGA
ATAGGCCATGACCACACAGGCTGTCGGCCATTTGATTGAAAGCACTGTTT
AGGCCAGGCCCAAAAAGGCTAGTCTTCCTTTCCATTGACACCACTGATTA
GGCCAGGCCCACACAGGCTAGTATGACTTTTGTTTGAGACCATGGATTAG
ACTAGTACCAAGACGCTAGACTGTCATTTGGCTGAGACCATGGATTAAGC
CAGGGTCACACTGTCTAGTCTGCCATTTGATTGAGAACATGGATTAGGCT
ACTACCAACAGGCTTGTCTTCATTTGACTGAGACCATGGATTTGGCCAGG
ACCACCCAGGCAAGTCTGCCTTTCGACTGACACCACTAAATAGTCCAGGC
CCACCCAGGCAGACTGACTTTTGATTGAGACTACAGATTAGGCCAGTACC
AAAAGGCTAGTTTGTCATTCGACTGAGACCATGGATTAGGCCAGGCCCAC
CCAGGCTAGTCTGCCTTTAAATTGAGACCATGAATAAGGCCAGGACCACT
GAGGCTAGGCTTCCATTTGTTTTAGGGCACTGCTTAGGCCAGGCCCACCC
AGGATAGTCGGCCTATCAATTGACACCACTGCTTAGGGAAGGCCAACCCA
GGCTAGTCAGCCTTTTGATTGAGACCACGGATTATGCCAGTACCAACAGG
ATAGTCTCTCATTTGACTGAGCCCACGGATTAGGCCAGGACCACCCAGGC
TAGTCTGTTATTTGATTGAGAGCACTGCTTAGCCCAGGATCACCTGGGCT
AGTCTGCATTTAGATAGAAACCAGGGATTAGGTCAGGCCTTCACTGGCGA
GTCAGCATATTGACTGACACCACTGATTAGGCCAGGCCCACTGAGGCTAG
TCTGTCATTTGACTGACATGAAGGATTAGCCAGGCCAATCCAGTCTAGTC
TGCCATTTGATTGACACCATGGATTAGGCCAGGACCTCCCAGGCTAGTTT
CCCATTTGATTGAGAGCATTGCTTAGTCCAGGCCCACCTAGGCTAGTCTG
TATTTTGATCAAAACCATGGATTAGGTCAGGCCCACACAGGCGAGTCTAA
CATTTGATTGAGACCACAGATTAGGCCAGTACCAACAGGCTAGTCTGTCA
TTTGACTGTGACAACGGATTAGGCCAAGCCCACCCAGGCGAACCTATCAT
TTGATTGAGCCCACGGATTAGGCCATTCACACCCAGGATAGTCTGCCATT
TGATTGCAACCAGTGCTTAGGCCAGGCCCACCCATGCTAATCTGCCCATG
AATTGAGACCACAGATTAGTCTACCCATTCTAGTCTAGTCTGCCATTTGA
TTGAGAGCACTGCTTAGGCCAGGTCCACCAAGCTAGTCTGCATTTTGATC
AAAACCATGGAATAGGTCAGGCCCACACAGGAGAGTCTGCATTTTGACTG
ACACCGCTGATTAGGCCAGGCCCACCCAGGCTAGTCTGCCTTTGAAATGA
GACCACGGATTAGGCCAGGGAAACCTAAGCTACTCTTGTATTTGTTTGAG
AGCAGTGCTTAGGCAAGGCCCCTCCAGGCTAGACTGCATTTCGCATGAC
CCAACTGATATGGCCATGCCCACCTGGGCAAGTCAGAGATTTGATTGAG
TGCATTGCTTAGGCCACAGCCAACAAGCTAGTCTGCGTTTTGATAGAA
ACCTGGATTCGGCCAGTGACAACAGGCTAGTCTGTCATTTGACGAGACCA
ATGAATAGGACAGGTGCACCCAGGCTAGTCTGATATTTGATTGAGAGCAC
AGCTTAGGCCAAGACCACCCAGGTTAGTCTGCATTTCAGATGACACCACT
GACTATGCCAAGTCCACCCAGGCAAGTCTGCCTTTTGATTGACACCACTG
ATTAGGCCTTGCCCACCCAGGCTAGTGTGCAATTTATTTGAGTGCACTGA
TTAGGAAGGCCTACCCAGGCTAGTCAGCCTTTTGGTTGACACCACTTATC
AGGCCAGGTCCATCCAGGTTACTTTGCCTTTGAATTGAGACCATGGATGA
GGCCAGTATCAACAGGCAAGTCTGTTATTTGAGCAAGAACATGGATTAGG
CGAGGCTCACACACACTAGTCTGCCATTTGAGTGATCCATGGATTAGCCA
TGCCCACCTGGGCTAGTCGGCCATTTGATTGAGAGCACTGATTAGGCTAG
GGAGACCCAATCTAGGCTGCATTTTGATTGACATCACTGATTAGGCCAGG
CCCACCCAGGCTAGTCTGCCTTTTGATTGAGAGTGCTGCTAAGACCGGTC
CCACCCAGGCTAGTCTGACTTTTGATTGAGACCATGGATTAGGTCAGTAT
TAACAGGCTAGTCTGTCATTTGACTGAGCCCACAGATAAGGCCAGGACCA
CCCAGGCTAGTTTGCGTTTTAATTGACACCACTGATTAGGCCAGGCCCAC
CCAGGCTAGTCTGCCTTTGAATTGAGACCACGGATTAGGCCAGTACTAAC
AGGGGAGTCTGTCATTTGAGTGAGAATATGGATTAGGCCATGCTCACCCA
GGTAGTCTGCCATTTGATTGAGAGCACTGCTTAGGCCAGGCCCTCCCAGA
TTAGTCTGCCTTTTGATTGAGATTACAGAGGCCAGTACCAACAGGCTAGT
CTGCCTTTTGATTGAAACCACTGATTAGTCCAGGACCACCCATTCTATTC
TTTCTTTTGATTGACACTGCTGGTTAGGTGAGGCCAACCCAGGCTAGTCT
GCCATTTGATTGACACCACATATGAAGCCAGGCCCACCCAGGGAAGTCTG
CCTTTTGACTGAGACCATGGATTAGGCCAGTACCAACAGGTAGGTCTGTC
ATTTGACTGAACACAGATTAGAACAGATCTCCCAGGCTAGTCTGCCCTTT
GATCAGCACCACGGATTATGCCAGGCTCACTCAGATGACTCTGCATTTTA
ACAGACACCACTGATCAGGCCAAACCCACCCAGGCTAGTCTGTCATTTGA
GACCACGGATAAGGCCAGTAACACCCAGGCTATTCTGCCTTTCACTTGAC
ACCACTGATTACGCCAGGGCCACCCAGGTTAGTCTGCCTTTCGACTGAGA
CATGGATTAGATAGGCCCACCCAGTCTAGTCTGCTTTTCAGTTGACACCA
CTGATTATGCCATGCCCACCTAGGCTTTCTGCAATTCAATTGAGTGCACT
TCTTAGGCCAGGCCCACCCAGGCTTTTCTGTGATTTGACTGAGCCATGAA
TTAGGCCATTCCCACCCAGGCTAGTCTATAATTTCATTGGGTGCACTGCT
TAGGCCAGGCCCGCCCAGGTGGGTCTGCATTATACTTTAGTCTGTTAGGC
CAGTACCAACAGGCTAGTCTGCCCTGTGATTGAAACCATGTATTAGGCCA
CGGCCCCACCCTCCAAGTTAGGCTGTGAATTGATTAAGAGCACTGCTTAA
GCAATGTCCACCCAGGCTGGTCTGCCATTTGACTGAGCCACCAATTAGGC
CAGGCCCACCCAGGCAAGTCTGCCTTTTGAATGAGACCACAGATCAGGCC
AGTATCAACAGGCTAGTCTGTCATTTGACTGAGCCTATGGATTAGGCCAG
GCCCAGCCAGGCTAGTCTGCCATTTGACTGAGCCATGGATTACACCAGGC
TCACTCAGATGACTCTGCATTTTGACTGATAGCACTGATCAGGCCAGGCC
CACCCAGGATATTCTGGCCACTTCAGTTAGAGCACTGCTTATGCCAGGGC
CACCCAGCCTATTCAAGCTTTTGATTGAGACCACAAAATATGCCAGTACC
AATAGGCTGGTCTGTCATTTGATTTTGACCATGATTTAGAACAGGTCCAC
CCAGGCTAGTCTGCCCTTTGATTGGCCCATATATTATGCCAGGCTACTCA
AGCAATTCTGCCTTTTGACAGACACCACTGATCAGGCCAGGCCCACTGGG
CTAGTCTGCTATTTGACTGAGACCACGGAATAGGGTTGGACCACACAGGC
TACTCTGCCTTTCGATTTACACCACTGATTAGGCCAGGCCCACCCAGGCT
AGTATGCCTTTTGTTTGAGACTATGGATTAGGCCAGTACCAACAGGCTAG
CCTGTCATTTGAGTGACCATGGAATAGGCCAGGAACACCCAGGATAGTCT
GCCTACTGACTGGGAGGGCTGCTTACGCAAGGCCCACCCAGGCTAGTCTG
CCTTTCGATGAAACCATTGATTAGGCTAGGTCCACCCATGCTAGTCTGCC
ATTTGATTGAAACCACATATGAGGCCAGAGCCACCTAGGAAAGACTGCCT
TTTGATTGAGACCATGGATTAGGCCAGTATCAACAGGCTAGTCTATAATT
TGACTGAGCCCACTGATTAGGCCAGGCCACCCAGGATAGTCTGCCATCTG
ACTAACCACGGATTAATCAGACTCATTCAGGCAACTCAGCATTTTTAATG
ATACCACTGATCAGACCTGGTCCACCCAGGCTATTCTGCCATTTTATTTA
GAGCACTGCTTATGCCAGGCCCACCCAGACTAGTTTGCCTTTCAAATGAG
TGTACTGCCTAGGCCAGGCCCACCCAGGCTAGTCTGCCTTATGATTTAGA
CTGATTAGGCCAGTACCAACAGGCAGTCTGCCCTCTGATTGAAACCATGA
ATTAAGTCATGCCCACCCAGGTTAGTCTGCAAATTAATTGAGTGCACTGC
TTAGGCAATGTCCATCCAGGCTAATCTGCCATTTGACTGAGCCACCAATT
AGGCCAGGCCCACATAGGCAAATCTGCCTTTTTAATGAGACCACAGATTA
GTCCAGTACCAACTGGCTGGTCTGACATTTGACTGAGAGCAGGATTAGAA
CAGGCCCACCCAGCCAGTCTGCTCTTTGACCAGCACCCAGATTATGTCAG
GCTCACTCAGGCATCTCTGCGTTTTGACAGACACCACTGAACAGGCCAGA
CCCACCCAGGCTAGTTTACCATTTGATTGAGACCATGGATTAGGCCAGTA
CCACCCAGATACTCTGCCTTTAGATTGAGACCACAGATTAGGAAAGGCCC
ACCCAACTAGTCTTCTTTTTGATTGACGGCATTGATTATGCCATGCCCAC
CCAAGCTATTCTGAGTTTCCACTGAGTGTACTGCCTAGGCCAGGCCCACC
CAGGCTAGTCTGCCTTATGATTTAGACTGATTAGGCCAGTACCAACAGGC
TAGTCTGCCCTCCAATTGAAACGATGAATTAGGCCATGCCCACCCAGGTT
GTTCTGTGAATAGATTGAGTGCACAATTTAGGCAATGTCCACCCAGGCTA
GTCTCCCATATGACTAAGCCACCAATCAGGCCAGGAAACCCAGTCAAGTC
TGCCTTTTTAATGAGACCACAGATTAGTCCAGGCCCACCCAGGCTATTCT
GCCTTTTGATTGAGACCACAGAATAGATCAGTACCAACCAGCTGGTCTGT
CATTTGATTGAGACCACAATTTAGAACATGTCTACCCAGGCTAGTCTGCC
CTTTGATTGGGCCCATGGATTATGCCAGGCTACTCAAGTGATTCTGCATT
TTGACAGACACCACTAATCAGGCCAGGCCCACCAGGCTAGTCTGCTGTTT
GATTGAGACCATGGATTAGGCCAGTACCACCTAGGCAACTCTGTATTTCA
ATTGACACCACTGATTAGGCCAGGCCCACCCAGGTTATTCTGCCTTGATT
TGGACCACGTTTTAGGCCAGTACCAACAGTACGGTCTGTCTTTTTATTGA
GATCACAGAATAGAACAGGCCCACCCAGGCTAGTCTGCCCTTTGATCGGC
ACCACAGATTATGCCAGGCCCACTCCTGTGACTCTGCATTTTGACAGACA
CCACCAATCAGACCAAACTCACCCAAGCTAGTCTGCCATTTGACTGAGGC
CACAGATTTGGCCAGAACCACCCAGGCTACTCTGTCTTTCTATTGATACC
ACATAATAGGCCAGGCCCACCCAGGCTAGTCTGCTGTTCAATTGACACCA
CTGATTAGGCCAGAACCAACCAGCTAGTCTTCCTTTGATCGACACCACTG
ATTAATCCAGGCCCACCCATGCTGTTCTGCGTTTTGACTGAGACCATGGA
TTAAGCCAGTACCAACAGGCTGGTCTGTCATTTGATTGAGACCATAGGTT
AGAACAGGTCCACCAAGGCTAGTCTGTCCTTTGATTGGCCCCATGGATTA
TGCCAGGCTACTCAGGCAATGCTGCATTTTGACAGACACCACTGATCAGG
CCAGGCCCACCAGGCTAGTCTGTCATTTGATTGAGACCACAGATTAGGC
CAGTACCACATAGGCTACTCTGCATTTCAATTGGCACCACTGATTAGGCC
AGGCCCACCAGGCTAGTCTGCCATTTGATTGAGACTATGGATTAGGCCAG
TACCACCTAGGCTACTCTGCATTTCAATTGGCACCACTGATTAGGCCAGG
TCCACCCAGTCTAGACTGTCATTTGACTGAGACTGATTATGCCAGCACCA
ACAGGCTAGTCTGCTATTGCCTTAGACCACGGATTCGGCCATGACCAACA
GGCTAGCCTGTCAGTTGACTGACAACAGAATAGGCCAGGACCACCCAGGG
TAGTCTGCCTTTCGATTGAGATCATGGATTAGGATAGACCCCCCACCCCG
CACCAGGCTATTCTGCATATCGATTGAGTACACTGCTTAGGCCAGACCTA
CCCAGGATCTTCTGTGATTTGACTGAGCCACAGATTAGGCCATTCCCACC
CAGGCTGGTTTACGATTTGATTGGGTGGACTGCTTAGGCCAGGCCCACCC
AAGCTAGTCTGCCTTATGATTTAGACTGATTAGGCCAGTACCAACAGGCT
AGCCTGCCCTCCAATTGAAACTATGAATTAGGCCATGCCCACCCAGGTTG
GTCTGCGAATAGATTGAGTACACTGCATAAGCAATATCCACCCATGTCCT
ACCATTTGACTGAGACCGTGGATTATGCCAGTATCAACAGGCTAATCTGT
CATTTCACTGAGCACACAGATTAGGCCAGGTCCAGACAGGCTAGTCTGCC
ATTTGACTGAGCCACAGACTATGCCAGGCTCACTCAGGCAACTCTGCATT
TTGACTGACAACACTGATCAGGCCAGGCCCACCTAGAATATTCTGCCACT
TCATTTAGAGCACTGCTTATGCCAGGGACACCTTGCTAGTCTGCCTTTCC
TTTGATATCACTGCTTATACCAAGCCCACCCAGGTATTCTGCCTTTTGAT
TCGGACCATAGAATAGGCCAGTCCAACAGGCTGGAATGAAGAGGAACTAA
AAAGCCTCTTGATTAAAGTGAAAAAGGAGGAGAGTGAAAAAGTTGGCTTA
AAGCTCAATATTCAGAAAACTAAGATCATAGAATCTGATCCCATCACTTC
ATGGCAAATAGATGGGGAAACAGTGGAAACAGTGTCAGACTTTATTTTTC
TGGGCTCCAAAACCACTGCAGATGGTGACTGCAGCCATGAAATTAAAAGA
CACTTACTCCTTGGAAGGAAAGTTATGACCAACCTAGACAGCATATTCAA
AAGCAGAGACATTACTTTGCCAACAAAGGTCCATCTAGTCAAGGCTATGG
TTTTTCCAGTAGTCATGTATGGATGTGAGAGTTGAACTGTGAAGAAAGCT
GAGCACCGAAGAATTGATGCTTTTGAACTGTGGTGTTGGAGAAGACTCTT
GAGAGTCCTTTGGACTGCAAGGAGATCCAACCAGTCCATTCTAAAGTAGA
TCAGTCGTCGGTGTTCTTTGGAAGGAATCATGCTAAAGCTGAAACTCCAG
TACTTTGGCCACCTCTTGAGAAGAGTTGACTCATTGCAAAAGAGTCTGAT
GCTGGGAGGGACTGGGGGCAAGAGGAGAAGGGGATGAACAGAGGATGAGA
TGGCTGGATGGCATCACCGACTCGATGGATGTGAGTTTAAGTGAACTCTG
GGAGTTGGTGATGGACAGGGAGGMCTGATGTGCTGCAATTCATGGGGTCG
CAGAGTCAGACACGACTGATCAACTGAACTGAACTGAACTGAACAGACTG
GTCTGTTATTTGATTGAGACCACGATTTAGAACATGTCCACTCAGGCTAG
TCTGCCTTTGATCAGCCCCACAGATTATGCCAAGCTACTCAAGCAATTCT
ACCCATCAGGCCAGGCCCACCAGTCTAGTCTGCCATTTGATTGACACCAT
GGTTTAGGCCAGTACCACCTAAGCTACTCTGCATTTCAATTGACACCACT
GATATGCTAGGCCCACCCAGGCTATTCTGCCTTTTGATTGAGAACATGGA
TTAGTCCAGTACATTGATTAGAACAGGCCCAATGAGCCTAGTCTGCCCTT
TGATCAGCACCATGTATTATGCCAGGATCACTCAGGTGACTCTGCATTGA
CAGACACCGCTCAATAGGCCAGGCCCACCAGGCTAGACAATGATTTGATT
GAAATAATGTATTAGGCCAGTACCACCCAGGCTAGTCTGCCTTTTGATTG
ACACTGATTAGGCCAGTACCAACAGGCTAGTCCACTATTTGACTGAGACC
ATGGAATAGGTCAGGACCACACAGGCTACTCTGCCTTTCCATTGACACCA
CTGATTAGGCCAGGCCCATCAAGGCTAATCTGTTGTTCGATTGACACCAC
TGATGAGGCCAGGACCACATAGGCGAGTCTGCCTTTGATTGACACCATGT
ATTAATCCAGGCCCACCCAGGCTATTCTGCCTTTTGATTGAGATCATGTA
TTAGGCTAGTACCAAGAGGCTGGTGTGTAATTTAATTGAGACAACAGATT
AGAACAGGTCCACCAAGGTGAGTCTGCCCTTTGATCGGCCCACGGATTAT
GCACAGGCTTCTCAGGTGATTCTGCATTTTGACAGACACTTCTGATCAGG
CNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
ATTATGCCAAGCTTCTCAGGCGATTCTACATTTTGATAAACACCACTGAT
CTGGCCAGGCCCACAAGGCTAGTCTACCATTTGATTGAGAACACAGATTA
TGCCAGTACCACCTAGGCTACTCTGCATTTCAATTGACACCAATGATTAG
GCCAGGCCCAGCCCGGCTAGTCTGCCTTTTGATTGAGACTGATTATGCCA
GTACCAACAGGCTAGTCTGCTATTGGACTGAGACCACGGAATAGGCCAGG
ACCACACAAAGTACTCTGCCTTTCAATTAACATCACTGATTAGGCCAGGC
CCACCCAGGATTGTCTGTCTTTTGATTGAGACACGAATTAGGCCAGTACC
AACAGGCTATATTGTCATTTGACTGACCAAGGAATAGGTCATGCCCACCC
AGGCTGGTCTGCAATTTGACTGAGCACCAATTAGGCCATGCCTATCCAGG
CTGGTTTGCTATTTGATTGAGAGAACTGCTTAGGCCAGCCTGGTCAAGGC
TAGTTTGCATTTCAATTGACACTACTGATTAGGCCAGGCCCACCCAAGGA
AGTCCACCTTTTGTATGAGACCATGGATTAGGCCAGTATCAACAGGCTAG
TCTATCATTTGACTGAGCCCACAGATGAGGCCAGAGGCACTCAGGATAGT
CTACCATTTGACTGAGTCACAGATTATGCCAGGCTCACTCAGGTGACAAG
GCATTTTGACTGACACCATTGATCAGGCCAGGTCCACCCAGGTTATTCAG
CCATTTGATTTAGAGCACTACTTATAACAGACCCACCCAGGCTAGTTTGC
CTTTCAGTTGAGAGAACTGCTTATGCTAGGCCCACCCAGGCTAGTTTACC
CCTCAACTGACACTGCTGATAAGGCCAGGACCACACAGGCTAGTCTGTCT
TTCAATTGACACCACTGATTAATCCAGGCCCACCTAGGCTATTCTCCCTT
TTCATTGAGACCATGGCTGGTCTGCAATTTGAGCACCAATTAGGCCATGC
CTATCCAGGCTAGTTTGCTATTTGATTGAGAGAACTGCTTAGGCCAGCCT
GGTCAAGGCTAGTTTGCATTTCAATTGACACTACTGATTAGGCCAGGCCC
ACCCAAGGAAGTCCACCTTTTGTATGAGACCATGGATTAGGCCAGTATCT
ACAGGCTGGTGTGTCATTTGACTGAGACCACGGATTAGAACAGACCCACC
TAGGCTAGTCTTCACTTTGATCGACACCGCGGATTACGCCAGGCTCACTC
AGGAGACTGCATTTTCACAGATACCAATGATCAGGCCAGACCCACCCAGG
GTAGTCTGCCTTTTGATTGAGACCACAGATTAGGCCAGTATGATCCAGA
CTACTCTGCCTTTCAATTGTTACCACGATTAGGCCTGGCCCATCCAGCCT
AGTCTGCCTTTWAATGAGACCACAGATTAGGCCAGTACCAACAGGCTGC
ACTGTCATTTGTCTGACCAGGGAATAGGCCAGGGAATAGTCAATTGGCTA
GTCTGCCAATTGACTAGGAGGACTCCTTCGGCTAGGCCCACCCACGTTAG
TCTGTCATTTGATTGATACCACATATGAGGCCAGGCCCACCCAGGGAAGT
CTGCCTTTTGATTGAGACCATGTATTAGGCCAATACCAACAGGCTGGTCT
GTCATTTGACTGAGAACATGGATTAGAACAGGCCCACCCAGGCTAGTCTA
CCTTACGATTGAGGCCACGGATTAGGATAGGCCCACCCAGGCAAGTCTGC
CTTTTGAGTAAGACCATGGATTAGGCCAGTATCAACAGGCTAGTCTGTCA
TTTGACAGAGACTATGGATTAGGCCAGGCCCAGCCAGGCTAGTCTGCCAT
TTGACTGAGCCACGGATTATGCCATGCTCATTCAGGCGACTCTGCATTTT
GACTGACACCACTGATCTGGCCAGGCCCACCCAGGATATTCTGCCTCTTC
ATTTTGAGCACTGCTCATGCCAAGCCCACCCAGGCTATTCTGCCTTTTGA
TTGACACCACAGAATAGGCCAGTACCAACAGGCTAGACTGTCATTTGATT
GAGACCATGAATTAGAACAGGTCCACCCAAGCTAGTCTGCCCTTTGATCG
GCACCACAGATTATGCTACTCAGACAACTCAGCAGTTCAACGACAACACT
GATCATGCCAGGCCTACCAGGCTAGTCTGCCATTTGATTCAGACCATGAA
TTAGGTCAGACCACCTAGGCTACTCTGCATTTCAATTGACACCACTGCTT
AGGCCATTCCCACCCAGGCAAGTCTGCCATTTGACTGAGAGCACTGCTTA
GCCCAGCCTGCACAGGCTAGTTCATATTTTCGATTGACACCACTGATTTG
GCCAGCCCCACGCAGGCTAGCCTGCCTTTTGACTGAGACTGACTAGGCTA
GTACCAAAAGGTACTTCTATTTCTACTTCTACTTCTAGTCTTCTATTTGA
CTAAGACCACGGAAGAGGCCAGGACCACACAGGCTATTCTGCCTTTCAGT
TGACACCACTGATTAGGCCAGGCCCACCTAGGCTACTGTGCCTTTTGATC
GAGACCACAAATTGGCCAGTACCAACAGGCTAGCCTGTGATTTGACTGAA
CATGGAATAGGTCAGGACAACCCAGCCCACCCAGGCAAGACTCCCTTTTG
ATTGAGACCACAGATTAGGCCAGTATCAACAGGCTAGTCTGTCATTTGAC
TAAGCCCGTGGATTGGGCCAGGCCCACCCAGGCTAGTCTGCCATTTGACT
GAGCCACAGATTATGCTGGGCTCACTCAGGTGACTCTGCATTTTGACTGA
TACCACTGATCAGGCCAGGCCTACTCAGGCTAGTCTGCTTTTGATTGAGA
CCACGGATTAGACCAGTACCAACAGGCTGGTCTGTCAGTTGACTGAGACC
ATGGATTAGAATAGGCTAGTAGGCCCTTTGATCAGCACCTCAGATTATGC
CAGGCTCACTCAGGTGAATCTGCATTTCAACACACACCACTGATCAGGCC
AAACCCACCCAGGCTAGTCTGCCATTTGATTGAGACCACAGATTAGGACA
GTACTATCCAGGCTACTCAGCCTTTCGATTGACACCACTGATTAATCCAG
GCCCACCCAGGTTATTTTGCCTTTTGATTGATACCATGAATTAAGCCAGT
ACCAACAGGCTGGTCTGTCATTTTATTGAGACCCTGGATTAGAACAGGTA
GACCTAGGCTAGTCTGCCTTTCGATTGAGAACACGGATTAGGCTAGGCCT
ACCCAGGTTAGTCTGCCATTGTTTGACACCACTCATTAGACCAGGCCCAA
CCAGGCTAGTCTGCCTTTCAATTGAAACCACATATTATGCTAGGCCTACC
CAAGCTAGTCTGGCTTTTCATTTACACCACTGATTACGCCATGTCAGCCC
ATGCTAGTCTGCAATTTGATTCAGTACACTACTTATGCCAGGCGTACCCA
GGCTAGTTTGTCATTTGACTGAGCCATGGATTTGGCCATTCCCCCCTGGC
AACTCTCCCATTTGATTGAGAACTCTGCTTAAGCCAAGCCCACCCCAGGC
TAGTCTGCCTTTTGATTCACACCACTGATTAGGCCAGGTCCACCCATGTT
TTTCTGCCTTTTGATTGAGACCTCAAATTAGACCAGTACCAACAGGCTAG
CCTGTCATTTGACTGAGACCACGGATTAGGCCAGGCCCACCCATGCTATT
CTGCACTTTGATAGGCACCATGGGTTAGGCCAGGCACACTCAAGCATCTC
TGCATTTTGATCTACACCACTGATTAGGCCAGGACCCCCAGGCCAATTTG
CCTTTCAAGTGAGACCACAGGGAGGCCAGTACCACCCAGTCTAGATAGCC
TTTGGTGGATTCCATGGATTTTGGCAGGCCCCCCCCACCGCCCCCCAGGC
AAGTCTGCCATTTGATTGAGAGCACTGCTTACACCAGGCCCACCCAGACT
AGTATGCCTTTTGGTTGACAGTACTGATTAGTTCAGGCCCACTCAGGCTA
GTCTGCCTTTTATTCAGACCTTGGATTAGGCTAGTATCAACAGCCTAGTC
TGTCATGTGACTGATCCCACAGATTAGGCCAGGACCACCCAGGTTAGACT
GTCATTCCAATGAGCCATTGATTAGGCCATGCCCACCCAGGCTAGTCTGA
CATTTGACTGAGAGCTCTCCTTAGCCCAGGTCCTCCCAGGGTAGTCTGCA
TTTTGATAGACACCACTGATTAGGCCAGGCCAACCAAGTCTAGTCTGCCT
TTCTATTGACACCACTGATTACGCTAGGTCCACCCAGGCTATTCTGCCCT
TTCACTGGGCCCAAGGATTATGCCAGGCTCACTCAGGCGACTCTGCATTT
TGAAAAGCACCACTGACCAGGTCAGCACCACCAAGGCTATTTTGCCATTG
GATTGAGACCACGGCTTAGGCAAGTCGACCCAGGCTACTCTACCTTTCAA
ATGACACCACTGATTAGGCCAGGCCCACACACACAGGCTAGTGTCTTTCG
ATTAACACTACTGCTTAGGCCAGGCCCACCCGGCTACTCTGCCATTGGAT
TGAGACCACGTATTAGGTGCATACCAACAGGCTAGCCTGTAATTTGACTG
AGACCATGTATTAAGCCAGGAACACACAGGCTACTGTCTTTCTATTGACG
CCACTGATTACGCTAGGCTCACCCAGGCTTTCTACCTTTCGATTGGGACC
ATGGATTAGGCTTTGCTCATGCAGGCTAGTCTGCCTTCTGATTGACACCA
CTGATTAGGCCATGCCTACTCAGGTTAGGTTGCGAATTGTTTGAGTATAC
TGCTTAGGCCATGCCCACCCAACTAGTCTGCCATTTGATTGTGAGCACTG
CTTTGGCCAGGCCCACCTAGGCTAGATTGCATTTCAATTGACACCACTGA
TTAGGCCAGGACAACCCAGCCATGTCTGCCTTTTGATTGAAACCACGAAT
TAGGGCAGTATCCACAGGCTAGTCTGTCATTTGCCCCACGGATTAGGCCA
GGCCCACCCAGGGTAGTTTGTATTTCAGTTGACACCACTGATTAGGCCAG
GCTCACCCAGGTAAGTCTGCTTTTCGATTGAAACCATGGATTAGGCTAGG
CCCACCCAGGCTAGTCTGTCATTTGATTGAGAGCACTGGTTAGGCCAGGA
CCACACAGGCTACTCTGCCTTTCTGTTGACAGCACTGATAAGGCTAGGCC
CACCCAAGCTTGTCTGATTTACGTTTGAGACCATGGATTAGGCTAGGTGC
ACCCAGGCTAGTCTGCCCTTTAGTTGGCACCACGGACTATGCCAGATTCA
TTCAGGTGACTCTGAATTTTGACAGACACCACTGATCAGTCCAGGCCCAC
CCCAGCTAGTCTACCATTTATTGAGACCATGGTTAGGCTAGTACTACCCA
GGCTTTTCTGCCTTTTGATTACACCAGTAATTAGGCCAGGCACACACAGG
CTAGTCTGCCTTTCAATTGACACAACTGCTTAGGCCAGGCCCAACCAGAC
TAGTCTGCCTTTTGATTGACACCGATTAAGCCAGTACCTACAGGCTAGTC
TGCCATTTGCCTGAGACCATGGAATAGGCCAGGACCACACAGGCTACTCT
GACTTTCAACTGACACTACTGATTAGGCCAGGCCCTCCCACATAGTCTGC
CTTTCAATTAACAGTGCTGATTAAGCCAGGTCCACCCTGGCTAGTCTGCT
TTTTGAGTGAGACCCAGGATTAGGCTGGGCCCACCCACGCCAGTCTGCCC
TCCAATTGACACCACTGATTAGGCCATGCCCACCGAGGCTAGTCTGCCAT
TTAACTGAGCCACATTTTATGCCATGCCCACCCAGGCTAGTTTGCCATTT
GATCGAGAGCACCGCTTAGGCCAGGCCCACCCTGGCTACTTTGCATTTCA
GTTGACACCACTGATTAGGCAAGGCCCACTCAGTCAAGTCTGCATTTTGA
TTGAGACCACAGATTGGGCCAGTATCAACAGGCTAGTCTGTCATTTAAAC
TAATCCCAAGGATTACGCCAGGCCCACATAGGATAGTCTGAAATTTGACT
GAGCCACGGATTATGCCAGGCTCACTCAGGTGACTCTGCATTTTGATGAC
ACTACTGATCAGGCAAGGCCCACCCAGGCTAGTCTGCCATTTGATTTAGA
CCACAGATTAGGCCAGTACCACCCTGGCTACTCTGCCTTCAATTGACACC
ACTGATTAGGACAGGACCACCCAGGCTAGTCTTCCTTTTGATTGAAACTG
ATTAGGCCAGTAGTAACAGGCTAGTTTGCCATTTGACTGAGACCATGGAA
TAGGCCAGGACCACACAGGCTACTCTGCCTTTCGGTTGACACCACTGATT
AGACCAGGCCCACCCACGCTAGTCTGCCTTTCAATTGACAGCACTGATTA
GGCTAGGTCCACCCCAGCTACTCTGGCTTTTGATTGAGACCATGGATTAG
GCCAGTACGAACAGGCTAGTCTGTCATTTGATGGAGACTGTGGATTAGGT
CAGGACACACAGGCTACTCTGACTTTATATTGACACCACTGATTAGGCTA
GGCCCACACAGGCTAGTCTGCCATTTGACTGAKGTCACGGATTATGCCAG
GCTCACTCAGGTGACTCTGCATTTTGACTGACAACACTTCAGGCCAGG
CCAACCCAGGCTACTCTGCCTTTCGATTGAGACCATGGATGAGGCCAGTM
CCAACAGGCTAGTCTGTCATTTGAATGAGACCACAGCTTAGGCCAGGACC
ACACAGGCTACTTTCCCTTTCATTTGACAACACTGATGATGCTAGGCCCA
CCCAGGCTTGTCTGCCTTTCGATTGACACCACAGATTCAGCAAGGCCCAC
CCAGGTAGTCTGCTTTTGATCAGCACCACAGATTATGCCAGGCTCACTGA
GGCGACGCTGCATTTTGACAGACACTACTGATCAAGTCAGGCCCACCGAG
GCAAGTCTGCCATTTGACTGACACGACAGATTAGGCCAGTACCACCCAGG
CTACTCTGATATTCAGTTGACACCACTGCTTAGGTCAGGCCCACCCTGGC
TAGTCTGCCTTCTGATTGAGACCACGGATTAGTCCAGTATGGCTCTTGAT
GAAAATGAAAGAGGAGAGTGGAAAAGTTGGCTTAAGGCTCAACATTCACA
AAACAAAGATCACGGCATCTGGTTCCATCACTTCACGGCAAATAGATGA
GGAAACAGTTTCAGACTTTATTTTTGGGGCTCCAAAATCACTGCAGAT
GGTGACTGCAGCCATGAAATTAAAAGACACTTACTCCTTGGAAGGAAAGT
TATGACCAACCTAGACAGCATATTCACAAGCAGAGACATTACTTTGCCAA
CAAAGTTCCATCTAGTCAAGGCTATGGTTTTTCCAGTAGTCATGTATAGA
TGTGAGAGTTGAACTGTGAAGAAAGCTGAGTGCCAAAGAATTGATGCTTT
TGGACTGTGGTGTTGAAGAAAACTTTTGAGAGTCCCTTGGACTGCAAGGA
GATCCAACCAGTCCATTCTAAAGATCAGTCCTGGGTGTTCTTTGGAAGGA
ATGATGCTAAAGTTGAAACTCCAGTACTTTGGCCACCTCATTCGAAGAGT
TGACTCATTGGAAAAGACTCTGATGCTGGGAGGGATTGGGGGCAGGAAGA
AAAGGTGACAACAGAGGATGAGGTGGCTGGATGGCATCACCGATTCTACA
GATGTGAGTTTGAGTGAACTCCAGGAGTTGGTAAAGGACAGGAGGCCTGG
AAGGTTCTGTGATTCATGGGGTCCCAAAGAGTCAGACAGGACTCAGGGAC
TGAACTGAACAGGCTAGACTGTCACTTAAATGACACTGCATATTAGGCTA
GCAATGGCACCCCACTCCAATACTCCTGCCTGGAAAATCCCATGGACAGA
GGAGCCTGGTAGGCTGCAGTCCATGGGGTCGCTCAGAGTCCCACAGGACT
GAGCGACTTCACTTTCACTTTTCACTTTCATGCATTGGAGAATGAAATGG
CAGCCCACTCCAGTGTTCTTGCCTGGAGAATCCTGGGGACGGCGGAGCCT
GGAGGGCTGCTGTCTATGCACAGAGTCGGACAGGACTGAAGGGACTTAGC
GGCAGCGGCGGCTGCCCTCCGATTGACACCACTGAAGGGGTCATGCCGAA
TCAGGTTAGTCGCGAATTGATTGACTACACTGCTTATGCCATACCCACCC
GCGCAGGAATGGGGGGCGGGGGGTGGGGAGGGCTCACTGCGCGTGCTACC
AGGGAAGGGCTGGCCCCGCACGAGCGCAGGAACGTGGCGGGGGAGGGAAC
GGGGGGTGGGGGGTGGCGGCTAGTGCCCCTCCACGCGCAGCAAGTGGGGT
TTTGCACCACCCATGCCAGGAGGCCTGGGCGTACTTAAGAAGTGGGGTCT
CACGTCGCGCAACGCACGCACAGTGAGAAGGCTGATACCGTGGCCCAGCA
AGAGGGCCTCGTGTCGCCTGTGGCGTGGGGCCCCGTGCGCGCAAGAAAGG
AGAGCTCGCGCCCTGCAGCGTGGGCAGAAAAGGGGGGCCTCGGGCCGCGC
GTGCCGGAGGAGGGCTGGCCTGCATGCGCAGTAAGAGGGCTCTCGCGCCC
CGCCGCGCTGGCAGGAACGGGGGTGCTTGCGCTGGCAGGAACGTGGGTGC
TTGCGCTGCCTGGGCAGGAAGGGGGGGTTTCGCGCCGCGCGTGCCGAGGA
GGGCGGGGGACTGACACCCGGCTCGCGCAGGAACCGGGGTCTCGCGCTGC
GCCGCGCGAGCAGGAGGTGGGGGCACCCGCAGCAGGTGGCGGGGGCTCCC
GCGTGCGCAAGAAGGGAGAACTCGCGCCCTGCAGCGTCTGCAGGAAGGGG
GTCTCGAGCTGCGCGTGCCGGTGGACCCTGCGCCCACAGGAAAGGGGGTC
TCGCCCCGCGCGCAGAGGAAAGGGGGGCACGCGCCGTTCGCGCCAGTAGA
AGGGCTGGTCCTGCGCGCGCGCGCGCGCGCGGGTTGGGGTCAGGGGCCGG
TACCCCCTATGGCTGCTACGACCTCCCGCCGCCCGTGGGGAGTAGGGAGC
TGCGGGCTACGGCTGCAGCTCGCAGCTCGCGGTGACCTGGAGGGGCGCGG
GGCTGAGTGGCGCTCCCCCTGGGGCCAGAGGTCCGGCCGGGGCGGCGGCT
CGAGGCCCAGCCGTTCCCAGCGTCCCCGGGCAACAGGATCGGCACGTGAG
GCGGCGGGAGCCCCCGTGTTGGCACCCGCGGCCGGCCGACTCCCCAGACG
TTGTTGGCGCGGAACCGGTGGGGCCGGAAGCGCCCAGGAGTCCCCGCAGC
GGTCCCGGGCCCACCCGCCATTCCCCTGTGCTCCTAGCAGCGGGCCCGGC
ATGCCGGGTCCCGGGAGCCCTCTGCGCGCGCCCCCGCGGCGTGGCCAAAG
CGCTGGCCGTCAGGAGGTGCTCGGAAGTCGGGCGGTAGGAGAAGGGGTCT
TGGTCTTCCACSGGGACTGCCCGTACACCCTCATGTATTAGCCGGCAGTG
CGGCGCGCAGCCCAAAGGCGGAATGGCCTCTGTTGGCGCTACTGTAGTTA
CCGCTTAGCGAGTCTTTGCTCCCAAACCCTGCTGTCGMTTTGGCACAAGT
GTGCGACAAGGACGTCAGCTGGTATACGACTGTTCTGCAGCTGGGCGCTG
GTGAATACCTGATCCTCGCCAAGGAGCTTCAGCCAAGTGTTGTGCAGGAA
GAAGGTAGCAAGGTCAATCCGGGAGAGCACAGACAGGACAGGGAGTCAGA
GGGCCTGGCAGGTCCCAGAGGAGGACGTGCCAGGACCTGCATCACAGTAG
GAGTGMCAGTGTCTTTAGGAGTCACGAGCGGGTTTWGTATTCTCCGGTTC
TGTGGCATCTTAHGCCGACCACTGGGGAAAGRCKGCCTGGACAAGTGGAC
AGGGGGACCGCCCCATAAAGGACTCCAAGCTTCAGTGACCGCAGGTCCTC
CGGGTAGAAGTATGAAACGACTTCTCCTTTGTAAAAGGAGTCTCCAGGAG
GGCGCCTGTGAGAAGCAGGCTGTGGTGGACTTTGGCTGTGGCGGGTCCA
TTCAAGTGCACAGAACTRAATACAGGCATGTGGTGGATGGTCCCAGC
CTTCACATCCTAGTTGGGAGAGTGTGCTCTATCATTCCGTTCAATGTTC
CCATTGGGAGAGGTGAGGCACGGTGCACAAAGGAGGTCCCTGTATCATTT
ATTTACAACTACATGAAAATCTACAACTGTCTCCAAAAGCAAAGTTGAAG
AAGTTGCTGGTGTAGGAATGCCATGGCAGTCCAGTGGTGAGGACTCCAAG
CCTCCACTGTGGGCGGCACAGGTTTGATGCTTGGTTCTGACCAAGGAACT
AAGATCTCATGTGCTGAGGAGCAGCTAAACCCGCGTATGCCACAGCTACT
GACCCCAGGCGCCACAAGTAGAGAGTCCAGGGACCACCCTAAACAATTTC
AAGTGCAATAACTGACACCTGACACAGCCAAATTAAAAAATGCCGAAGTC
CAACTCCAGTAACCAGGGATTCAACCTGAAGAGATGACCGTGTCAGCCAA
CAAGACAGCCTCTCAGTTTTCTATGGACTGCCTGTTCATTCCAAGTTTAA
GTTTCTCTTTTATACTTTTACAAAAACATTAGGCTAGAGGTTTGACATTT
TCAGTTCCCCCTCTCCCAGATTTATTATCTCCATAAATCATTGTCGCTGT
TCCTGCTTCAGGGATTCTCTCCGGAATCAGCCTTATCATCTATTATCTAC
CTCCTCTAATGTGTCCTATAGTTAACTTGTGATTATATTGTAACTCATGC
CACATTCCTCAGTTTACTACTTATCTTCCTAAATCCTGTTTGCCCCTAAC
ATCCTGAGCTCACTATCTCTTAAAAAGGCTTCTTAGCTATAATGTCTCTA
AAAAATTCCTAAATTCTATAAGCTATAGTAAAATATGCTAACTTTACAAC
ATTCCTTAAATCTTTAACTTCTATTTTAATTATTTCTAAGCCCTAAATTC
AGTAAACTCCTTTGCCATAAACTTTGTCTTCACAAATAGGCTTTAGATAT
CAATGCCTCCCATGGCCTCAAGCTACGGCCTGTGTGCTCATCCTGGAACA
CTCTTTTGCACAAGTTCTTTAACAAATGTCAGTGATTAACTTTATGAATT
ATTTTCTGAGCACAGCTGCAGAAGGCTTTGTGCCTTCTCATGCTCCTCTC
AAGAACAATAAGCACCTTAATATTCCTTTTCAGTGAACTCAGCCGACGAG
AGGAAAAGACAAGTCAGAATTACAAGGCCTAACTCCTTCATCCTGGGATC
CATGCCTGCGGAATGAGGAGAGGAGTCTGGGGCTGTGCCTCCATTTTGTC
AGTAGTGCCTAACGTGGCTCCTGACAAAAAAAAATAAAAAAATTTTAAGT
GGATGGATTTGATCAATTGAAGGCCTCAGAGGTGGCACTTGTGACAAAGA
ACCCGCCTGCCAAAACAGGAGATGCAAGAGACACTAGGTTTCATCCCTAG
GTGGGGAAGATCCCCTGGAGAAGGGCATGATAATCTGCTCTGGTATTCGT
GCCTGGAGAATCCCATGGACAGAGGAACCTGGAGGGTTACAGTGCATGGG
ACCACAAAGAGTCAGACCTGACTGAGTGACTAAGCACATGATCAATCATA
AGGTGAAAGACCATATAAATGCCCCTAGAGTTTTTGGATGTGCCCTGTCC
CAACTCGGGGCTTCCCTTGTGGCTCAGCTAGTAAAGAATTCTGCAGTGGG
GGAGACCTGTGTTCGATCCCTGATTTGGGAAGATCCCCTGGAGGAAGGCA
TGGGAACCCACTCCAGTATTTTTGCCTGGAGAATCCCGTGGACAGAGGAA
CCTGTGGAGTACAGCCCATAGGATGGCAAAAACAGACAGACATGGCTGAA
GCAACTTACCACACATAGCATCCTAACTAATCACCTAATGCCATCCTTCT
AGTAGGAATTTTCTGTCTTGAGACTATAAAAATGGGCTGCTAGCCCATCA
AAGGGGTCGGCTCTCCCTTGACCGGCCCCCTGTTCTAACAGCATATCCCA
TACTGCACTCCGATACACTCTCTTCTCCTCTCATTCTGCCTCATCTCTGG
AAACTTTTCCARCCSGTGCACRGACCACCACACTCCCCCAAAACACACTT
CCTTCARAGTGCRGTGACATTCAGATGCCATCACATCTGAGACCACCTGC
GACCTGCCTCCAGCCTCTCAAAAGTGGACTTCTTCTTGCTTCGAGATGCT
GAGTCACAAAGAGTACTTCGGTCTGTCTGACCCCTTTGCGGACACTCACA
TTGACGGGGCGGTGAACCTCGTATTCCCTGCTGGAGCTTAGGGAGTGACC
TGCCTGGATTGAAGTCTCAGCTCTACGACCTCTGTGAGCTTGGACACGTC
ACATGTCTTCTGCTGCCTTGGACAGAAACTAGAGATAGAAAGGTGAGCCA
CCAGGATAGGGGGTGCCAGTTAAGCCAGACTTGAAGTTATCCAGTGCAGC
CAACACTGCACTGGAAGGCAGTCGGATAAGGATACTCGCGTTAGCTTGGG
TTCCTTCTTTGTCTCTGGACACAAACCCCACCCTGTCCTTTCCAGCCTGG
GCTCGTCTCTTTGGAGGCTGAGAAATGGAGCTCAAGACTTAGTCAGATGG
YTCCAGCATTGAATGTTCTGCTGACTGCTCTCTTAGGCACTATTTCTCAA
GCCATCCATTTCTGCTATCTCTTCAGAACAAAGACTGGGTTTCCTTCAGG
AAGCCAAGTCAGGTGAGGTTAATTCCCCACAAAAACCACAAGTCAGACTC
TTTGGCTTCTAACTCTCAGTAGCTCACTCATAGTCTGACTCTTTGTGACC
CCATGGCCTGTAGCCTGCCAGCTCCTCTGTCCACGCAATTCTCCAGGCAA
GAATCCTGGAGTGGCTAGCTGCTGTTCCCTTCTCCAGGGGATCGTCCTGA
CCTCTGATGGAACCCTGGTCGCCCGCATTGCAGGCAGATTCTTTACCTTC
TGAGCCACTAAGCCCAGGTTCAAAACCCACCCACTGTGTGCAATATTTAC
AGAGCTACTGAATTCCCCCGGGGTGGGGGTTGGGGGGCAGAAAAAGAGTT
GCATGCCAAGCTGTAACCCACGACAACACTTTTTTATCCGCTTAAAGCTC
TGTAGGCAACCTTGAGCAGTTTTGTCCGTTCTGGAGACACTGGGCAGAAA
CAGAGGCCGAAATGCAGTGACGCTGTTGCACAGATCCACCCCCCAAATCT
TTGGCATCAGGGCAAATGGACAAACGCAGCATTTCCATCTTTTAAGGCAC
GTTCCACACACGATCTTCCAAAAGAATGTTCTGCTTTCCAGGAGCCAAGG
AAATAGAAGATCAACTGTTCCAAACAGGTACTGAGATCTCCACTCTCTGA
AGGACTCAGGGTCTTGGGAGTAGTCCTATGATGGTTTGTCCTGTGTTTAC
AAGCAAGTGCATACTCTCCAAGGGTGGTTAAAGTTTCAACACCATCAGTG
TGTGTTCTGACTTCTATCAGAGCAGCTGCCCCTGCTTCCAGACAAGCCAG
CCTGGATCTTTCTGAAGTGGTAGCGTTTGCAGCTCCTGGTTAGAGGCCCT
GCTTCCATCTCGCAGTGACCTCCCCTCTGCCTTCCACCCCAACRGAAGCC
CAGTCCTGTGGTTTTCTAGGAGGAAGGCTGAGGAGCTGAGGCCAAACTCT
GTCTTATGTAAAACTGCAAAGCTGGTGGTGGAAATGGAGATGAGCCTCTG
GCGTGGGAGACGGTGGAGGAAAAGCATAGGGATGCCTTACATCTGGTTGG
AAAAATTCTGAGACCTCATGTTTACTGCTGGGAGAAGGTCAAAGACTGTC
TACTCCTGGCTGCAAATGGACCTGTTAAATTTCCTGATGGTAAGTATGAT
GGAGCTGGTGTTTTTCTTTCTTTCTTTTTTTTTTCAAATTTTGATTAGTG
AAGAATCTGCCTGCAATGTGGGAGACCTGGGTCCGATCCCTGGGTTGGGA
AGATCCCCTGGAGAAGAGAAAGGCTACCCACTCCAGTATTCTGCCCTAGA
GAATTCTATGCATAGTATAGTCCATGGGGTTGCAAAGAGTCTGACASGAC
TGAGGGGCTTTCACTTTCACTTTTAGTAAAGCCATAACTTAGATGAGGCT
CTTAGATTTTCAACTTGAAGACTTTTTTTTTTTTTCAAGTCCACTCAATA
TATTACCAATAGTACTAATATCATTTTGAAACTATTATGCGTATAGAATT
AAGCATGACTTTCAATGTTTAATGCAGTTCCTCTAAAAATTAAAGGAGTT
TGGCCTCCCTGGTGGCTAACTTGGTAAAGAATCCACGTGCAATGAAGGAG
ACCTGGGTTCAATCCCTGCGTCCGGAAGATCCCCTGGAGGAGGGCATGGC
AACCCACCCCAGTATTTTTGCCTGGAGACTCCCATGGACGGAGGAGCCTG
GCGGGCTACAGTCCACGGGGTCGCAAAGACTCGAACAGGAATGAGCGACT
AATACACTTTAAGAAAATCTGAATAAACTTGAACTTTTTTTTTTTTCCTA
AAGAGAGTTACTCTTGAAAGGTAACATACAATGTCAAAAATCTAACCTTA
CATGGTTGAGATGAGTAAAAATTGAGCGGACCTGAAGGTATAAAACGTGT
GTGTGTATGTGTGTGTGTGTGTCCAGTTTGGCAAAAGAGTTTCCCCTTTC
TTCAAAGTTTTGTTGATTCCAAGCCAAAGTCCTAGCTCTGTGGGGTGATT
TCCCTGGCGGAGGGGGGCCACAGAAAAGCAGTATTTTCATGCTAATCACG
GCAGGGTCACTGTTTATGAACTGGCAACGGATCAAAATGAAGGATGTCAG
AGACTCAGACTGCTCAGACCTGGCAAGAGCGCGTGGTTTCTGGGGGAGAT
GGGTTAAGATGAGACAGCACTGTCGAGGGCTCCGGGTTCTGGAGAATTAA
GCAGCCCCGCCCCCTCCCCCTCCCGGGAAGGCATGCCGACCGGCAGGAGA
GCGGACTTCCCGACCCTGAGCTCTCTGCCCCCACCCCTCCTCCTCGTTAC
CAAGCATCACCCTCGTGAGGCCTCGTGGCGTTCCGCGCTGCCCTCGCCGA
GCCTGGCGCCTTTTCTGCCAGCGCGGGGCGGGGATCAGGCGGGGGCAGCG
GGGAGGCCCAGGGCACATGACGCCCCCTCCCCGCGGCCCCCGCGCCCAGC
ACATGACTCAGGCCGGCAGGCAGACCCGAGCCACGCGCGTCCCCAGCACG
ACCCATGGCCTCTCCGCGACTAGGCACCTTCTGCTGCCCCACGCGGGACG
CCGCCACGCAGCTCGCGCTGGGCTTCCAGCCGCGGGCTTTCCACGCGCTG
TGTCTGGGTAGCGGCGCGCTCCGCCTGGCGCTCGGCCTCCTGCAGCTGCG
GCCCGGGCGCCGGCCCGCGGGCCCCGGGATCGCCTCAGCCTCGCCGGCGA
CCTCGGCCCGCGTCCCCGCCTCCGTGCGCATCGTGCGCGCCGCAACCGCT
TGCGACCTGCTTGGCTGCCTGGGTGAGCGCGCGAGCCGCGCGGGCGAGGG
TGGGTGGGGAACCCGTCGGTGTTCGAGTGAGGGCGCCCGGTCCGCGGGTG
GGGACCCCTTCTTGGGAGGTGAGGGCGCACGGCCGGGGACCCCTCCGTGC
GTGGTTCAGGGCCCGTGCGTACTGCACTGATGGAGACCCCTCCGCGCGCA
GTTGAGTGCGCGCGGCCCGCGAGTAGGGCTCCCTCTGCGGGTGAAGACGT
TGGGCCCCTAAGTAAGGGCGCGTCTATGCGTTTTTTCAGGGCGTGCCTAG
GTGCGCGGTTGAGAACCCGCGGCCCACGGGTGGTCCTCCGTGGGAGTTTG
ACGGGGTGAGGCCCGCCAGTGGGGACCCCTTCATGGGAGTTGTGGGCGCA
CGGCCCGTCGTAGGTGGGGACCACTGAGTGCGCGGTTGAGGGCGCGCAGC
TCAAAGGTGGGGACCCCTCCGTGCGCAGCTGAGCGCGAGCGGCCCGCGAG
TAGGGCTCCCTCAGTTTGTGGGTGAGGACGCCCGGCCCCCAAGCAGAGAC
CCGTCTGTGCGTTTTTTCAGGGCTCGCCTACGTGCGCGGTTGAGAGCCCC
GCGGCCCCACGGGTGGGCCTCCGAGCGAGGTTGACGAGGTGTGGCCCGCC
ACTGGGGACTCCTTCGTGGGAGTTGGGGGCGCACGACCCGTCGTAGGTGG
GGACCACTGCCTGCGCGGTTGAGGACGCGCGGCTCTACGGTGGAGACCCC
TCCGTGTGAAGGTGAGGACGCCTGGCCTGCGAGTAGGGACCGTCTGTGCG
TTTCCTAGGGCACACGCACCGCGGGTGCGGACCCCTCCGTGAGCAGTTGA
GGGCCCGCGGCCCGCGAGTGGGGATCTTTCCGTGTGTGCTTGAAGACGCG
CTGCCCGCGAGTGGGGACCCCTTCGTGCGCGGTTGAGGGTGCACGGCACT
GTGTGTGCAGTTGAAGGCACACGGCCCGGGGTGGGAACCCCTCCATGCCC
ATTTGAAGGCCCATGGCCCACGAGTGGGGACCTCTCTGTGCCCAATTTAG
AGCGCGCGGCCCGCGAGTGGGGACGCCCTTCGTGGGAATTGAGGGCGCAC
AGCCTTGCGGTGACAACCACTCCGTGCGCGGTTGAGGGCACATGGCCTGC
CGGTGGGGTCCCCTCCATGTGCAGGTGAGGACGCCCAGCCCACAAGGAGG
GACCCATCTGTGCGTTTTTCAGGGAACGAGTACCGCGAGTCGGGACCTCT
CCGTTCCCAGTTGAGGGTGCGTTTCCGGTGAGTGGGGACCTCTCTGTACG
CGGTTAAGGGCGCACACCCCGCGGGTGCAGACCCCTCTGTGCGCGGTTGA
GGGCGCACAGCCCATTGGTGGGGACCACTGTGTGTGCAGTTGAGGGCCCG
TGGATGCGGGTGGGGACCATTGTGTGGGCAGTTGAGTGTGCACGGCCCGC
GGGTGGGGTCCCCTCCGTGTGTGGGTGAGGACACCAGGTCCACGAGTAGG
GACCCATCTGTGCATTTTCCAGGGGTCGATTACCATGGGTCGGGACCCCT
CCGTGCCCAGTTGAAGGCCAATAGCCAGAGAGAGTGGAGTCCCCTGCGTG
TGCGGTTGAGGGCGCACGGCCTGCAGGTGGGGACCACTGCCTGCGCGGTT
GAAGGCGCCAGTCCGCGGATGGAAACCTCTCTGTGCCCAGCTGAGGGCGA
ATGGCCTGCTAGTGGGGACCTCTCTGTGCCCAGTTGAGAACGTGTGGCCC
GCGAGTGGGGATGCCCTTCATAGGAGTTGAGGGCACACAGCCTTGTGGTG
AGGACTACCACTCTGTGCGTGGTTGAGGGCACACGGCCCACGGGTGGGGA
GCCCTTGGTGCTCAGTTGAGGGCACGTGTACTGCGGTGGGGACCCCTCCA
TGCCCAGTTGAGGGCCCAGGAGTGGGGACCCCTTTGTGTGCAGTTGAGGG
CGCACGGCCTGCGGGTGGGGTCCCTTCCATGTGCGGGTGAGGACGCCCAG
CCCATGAGGCCCCTTCCGTGTGTGCTGAGGGCACGCTGCACGCTAGTGGG
GACCGCTCCCTGCGCAGTTGAGGGTGCATGGACCGCGGGTGGGAACCACT
GTGTGAGCAGTTGAAAACACACGACCCAGGGGTCTGCGAGTGGGGACCTC
TCTGTGCCCAGTTGAGAGCTCGTGGCCCATGAGTGGGTCCTTCATGGGAG
TTGAGAGCGCATAGCCTTGTGGTGAGGACCACTCCGTGCGCAGTTGAGGG
TGCACAGCCCACAGGTGGGGAACTCTTGGTGTGCAGTCGAGGGCGCGCCT
ACAGCCTTGAGGACCCATCCATGTGCGGTTGATGGTGCGTGGCCCGTGAG
TGGGAACCCTCTCTGTGTGCTTGAGGGCGCACTGTCTGCGAGTGGGAACC
ACTGCGTGGGCGGTTGAAGGCGCACGGCCCGTGGGTGGGAAGCCCTCCGT
GTGCAGGTGAGGATGTCGGGCCGCGAGTTGGGACCCGTCTGTGCGTTTTC
CAGGGCGCACGTAGTGCGTGTCGGGACCCCTCCATGCCCAGTTGAGGCCC
GCGGCCCTCGAGTGGGGATACCTCCATGTGTGTTTGAGGGTGCACTGCCC
GCGAGTGGGGACGCCTCTGTCCGCGGTTAAGGGCGCACAGCCCAGGGGTG
GGGACCACTGCGTGCGCGGTTGAGGACACCTGCCCTGCGAGCAGGGAGCT
GTCTGTGCGTTTTTCAGGGCATGCGCACCTCGGGTCAGGGCTCCTCTGTG
TGCTGTTGAGGGTGTGCAGCCAGCGAATGGGGACCTCTCCTTATGCGGTT
GAGGGCACACGGCCCGCGATGCGAACCCCTCCATGCCCAGTGGAGGCCGC
GGGGCCCGAGAGTGGGGACCCCCTTCCTGGGAGCTGAGGGAACACGGCCC
TCGATTGGGGAAGCCTTCGTGTGCAGGGAAGGACACCCGTCCCGGGAGTC
GTGTGCATGGTTCAGGGTGTACGTACCACGGATGGGGACCACTCCTTGCA
TGGTTGAGCGCACCTGGTCTCCGGGACTGGGGGACCCTCCATATGCGGGT
GAGGACGCCAGGCCCGCGACCCGTTTGTGAGTGGTTGAGGGTGCCCGGCA
GGCGGGTGAGGTCCCCTCCGTGCAAGGATGAGGGCACCCCGCCCACAGGT
GGGGACCCGTCTGTGCGTGGTTGAAGGCATGCGGCCCGCAAGTGGAAAGC
CCTCGGTATGAGTGGGGGTGCCCAGCAGTCGGGTGRTGCATGTTTTAGGG
CCTGTGGCCCATGAGTGGGGACCCCTACGTGTGCAAATGAGGATGCCTGC
CCCAGGGAGCGGGTACCGCTTGTGCGTGGTTGAGAGCGCCTGGCCTGCGG
GTGGGAGGACTGATCCAGAGAGGGCTACCCAGGGACACTACGAGGACGAG
TGGATTCTGCAATACAGTATGGGTAAAAGTTGCTTCCGGAGACTTCTTTT
TTTCTTTTCTTTTCTTTCTTTCTTTTTTTTTTTTTAACACTTTGATGTAT
TTGTTACTGCTCTTACTAGGGAAACTTTAGGCAGTGCTGTGAAGGAAAAC
ACAGAAAATACCACGGTTCCATGATCGTTCCACCAATGAGAGACTAATAG
TTAATATTATCAAGCCTGTGCTCACATACATGCAGTTGTTTCCAAACCGA
TTTAGCAGAATCAGGAAGAAAACTCCATATATACGATAAAATTTTTATTT
CCTTTTTTATCTATGTATCTAAATTTGGGTTTTCTATCACAAATGTGCAA
ACTTGTAGTCTCTAGGCTAAATCCAGCCTGCTTTTCTATGTACAGCCCAT
GAGTTAAGAATGTTTTTACATGTTAAAGTGTGTTTTAAAAATCTACCAGG
GGGCTTCCCTGGTGGCTCAGACGGTAAAGCATCTGCCTGCAAGGTGGGAG
ACCCGGGTTCAATCCCTGGATCGGGAAGATCCCCTGGAGAAGGAAATGGC
AACCCACTCCAGTACTCTTGCCTAGAAAATTCCATGGATAGAGGAGCCCT
GTGGGCTACAGTCTATGGGGTCCCAAAGAGTCAGACACGACTAAGCAACT
TCACTTTCTCCCTTTCTAAGAGGAGAGTGAAAAAGCTAGCTTAACACTCG
AAGCCTAGAAATCTACAGAACAGGGACTTCCCTGGTCGTCCAGTGACTCA
AACACTGTGCTCCCAATGCAGGAGACCTGGGTTCGATCCCTAGTCAGGGA
ACAGGATCTCACATTGCGGCAACAGAGACCTGGCCCAGCCAAGTAAATAA
ATACTTTTAAGAACATCTACAGAACAATGGTATTTCAGACATGTAAGCAT
TCTGTGAAATGTGAATGTCAGCGTCCGTAAGTGGACTCTTATGGGGATAC
AGCCGTGCCCTGCACTTGTGCACTGTTGGGGCAACATCTTCCACCCACCC
CATTTCCCCATCAGGGCCGAAATGAGTGTTGCATAGAGAAACATAGAGAA
AGTGTGGGCCACAGACCCTAAAATATTTTCTCTCTGATCCACTACAGAAA
CTTTGCTGACCCCTGCTTTCAACCCTAGCACGGCACGTTTTACAAACTCT
AAGCTCGATACTGCTGACCTTTTAAACTTTTTTATTTTGGACTTTATTAT
ATATATGCAAATGTAGAGACTGTATAAGAAGCCGCCTGTTCTCATCACCT
AGCCTCCTCTATACTTCATCCCTGTACCCCACTCCCCACTGAATTAATTT
GAAGCAGATCCCAGAATTTATATCATTACATCCAGACAAATCTCAGTATG
TAACTGTAGAAGAATTCAACATATATATAATTATATACATAATTATAATA
TATAACATGTCCATTGTAACATACTGTATACTACATGTGTGTGTATGTTA
GTCACTCGCTCATGTCTGACTCTTTGCAGCCCCATGGACTGTAGCCCGCC
AGGCTCCTCTATCCGTGGGATTCTCCAGGCAGTAATACTGGAGTGGGTTG
CCATTTCCTCCTCCAGGGGATGTTCCCAACCCAGGGATCAAACCTGGGTC
TCCCACACTGCAGGTGGATTCTTTACTATCTGAGCCACCAGGGAAGCCAT
ATACTATATGTTACAATATTATAATATATTACAATATATAATATATTTAA
TATGTTATATTAGTGAAGTGAAATCGCTCAGTCATGTCCAACTCTTTGCG
ACCCCATGGACTGTAGCCTACCAGGCTCCTCCGTCCATGGAATTTTCCAG
ACAAGTGTACTGGAGTGGGTTTCCATTTCCTTCTCCAGGGGGTCTTCCCG
ACCCAGGGATCGAACCCGGGTCTCCTGCATCACAGGCAGACACTTTACCC
TCTGAGCCATCAGGGAAGCCCAATATGTTATATTAAGTGTATATTTAATA
TAGTCTCATATTATAATATGTAATTATATATATATATATATCACACTGAT
ATCATTGCCACCTAAAAAATAAGGAAAACCGATTCCTTCATGTCACCGCT
ACATGTTCAGGCATGTCATACATGCCAGTTTTCCGTTCGTTAGTTCAAAT
CAGGATCCAAAGAGCGTCCGTGTTCTGTGAGTGCTGACGGGTGCTGGCCA
TGTTGGAATCGTCTGCTCTTGAAGGAGAGGCTCTGATGGTATTTGAATGG
CTTCCACCTCCGTGTCCTGAGGGTGTCGCAGCAGCTTCTAGCCCACCTTG
ACCAGATACCTGGTGGATGTGTTCCAGGGGTTGGCTCCCCTGTTCCTCTT
GCTGTTTTGTGCTTGGAGACACTCAAGAATCTTGACTCCAGACACATGCT
ATGGAACTTGCAGTCACTTTTTCAGACTGGAGTAAGACAGGAAGGAATAC
CTCCCTTTCTGTTAGTGACAAAGCGAGTGTGGTGCAGGATAATTAAATTT
TAAAAAATTGGTTTTCTCCCTGAYTCTGCTGAAGCATTTGGGGGAAGCAT
CTTAATAGTTTCAGTAACTTGAATATCTGGAGATAGAGTTGCAGCCAAAC
ATCTTTTGGGGGGTGGGTCAGGAAATAAGATTGGTACATCCTTTCACCTT
TTGAAGGAAGGTACATGGCACTGTGGGCAAATGAGCAGAAGCAGGAGTCA
GGGCAAGGCAGGGGGTGTCTGGACCTCCTCGCTGAAAAATAACAAGCACA
GATGCCAAGGTGTCCCTGGTGGTGCAGTGGTAAAGGACCCGCCTGCCAAT
GCAGGGGACATGGGTTCGATTCCCGGTCCAGGAGGGTCCCGCGTACTCCA
GAGTCTGTGAGCCACAACTGCTGAGCCTATGCTGCAGAGACTGGGGTCCA
TAACATCTGAAGCCCACGACCCACCTAGAGCCTGCGCTCCGCAACAAGAG
ATGCCACGGCGATGAGAAGTCCACATACCACAATGAAGAGTAGCCCCCAC
TCTGAAACTAGAGAAAGCCTGCAAAGACCAAGTGCAGCCAAAATAAAGAC
AGAAAGAAATACAGATGCCTATTGCATGGAATGTGATTCTGCTCCCCCAC
TACCCCACCCCCCCCGGAGGATTTTTTAAAATCTTTTTTTTTTTTCCTGG
ACAAATTGCACAGCTTGTGGGATCCCAGTTCCCTGACCAGGGATTGAACC
TGGGCCATGGAAGTGAAACCACAGAATCCTAACCACTAGACCACCAGGGA
AGTCCCGCCCCAGTTTTGCTGACACTTCACTGACATACAGCACTGGGAAA
CGTTTACATATCCAGCACAATGACTCACATTTACCATAAAATGTTTCCTA
AACACCCATCACCTCATATACATGCAAATATATATACATATGGTGTTTAC
TGAGCTCAAAGCAGATCTCCATAGTGAAAACAACAAAGCACTATTTGGAA
GAAAACGTCATTTCTGTTATTTGCACTTAAGAATAAGAAGCGACATCTTT
AGGATTTTAGTGTCCATAATGCCCTTTGGTGGTGTAGGACACAGTGTATA
TCTGGTTGCCTATTTTGAAGTATATGTTTTGTATGCCAACTACACACATT
ATGCTAGTATACAAGTCTGTACTGTTCATTTCCCCTTGAGCATTTTGTGT
TATACACTGTACACTCAAACAGATGCTATCCCAAACCTCACAACCCTATT
ATATACGTTGAGTGTGGATACTGATTATAGGCGAATTAGATATCTGGATC
TAAGAGCAAGGAACACTCCCAAATAATAAATGTCAAAAGTAGATTACAAG
AGCTAACTTGTGAAAATCTTCAATTTTTAATTTTATTAAAAAGAACAGAA
GAACTCTCTTTCACAACTTCAGAATAAGCTTTTTCGGGACTTCCCTGGTG
GTCCAGTGGTTGAGAATCCACCTTGCAGTGCAGGGCACACGAGTTCCATC
CCTGGTCAGGGAAGATCCCACATGGCGTGGAGCGACTAAGCCTATGCACC
ACAAGTTCGGAGCCCACGAGCCATGACTACTGAGCCCAGGTGCCGAAACT
ACTGGAGCCCAGGTGCCCTGGAGCTGGTTCTCCGCAGCAAGGGAAGCCAC
AGAGATGAGCAGCCCAAGAGTAGAGGCCCCTGCTCGCGGCACCTAGACAA
AGTCCTGTTAGCAGCAACAAAGACCAATCAATAAAATAAATCAATACATC
CTTTTTTAAAAAAATTAAAAAAGAAAGAATGTGCTTTTTTTCAGTCGTTC
TTTTTTTTTTTTTTAACATGAGGACTAGTTAAGCCGGACACTAAAAATAC
AGCCAAGAAACACACACTGTTATTCACACAGTTGCTTCTTAGAGGCAAAT
AAACGACCAACCAAAATAAGGAACATGACTCTAGCTGATGTTAGTGGAAG
TGGAAATCAAAGCGCCGGGGATTGCTCTGCAACCATAATCCTTATCTTCT
GAGTCTTTTTTTTTTTCTCCTCTGTCTCTCCTGTACCCACCAACATGTGT
ATCTTTCATCTCTGCCTCCCTCACATCTTTCTCTCTCCCTTCCTATCTTC
TATCCATCCATCCTTTCATCTGTCCATGTGTCCATTTGTCCATCCATCAT
TTTTACAGTCAGCAGACCCCCTTTAGGTCTGAGTCAGGAGCGGACACAAA
GAAAGCGTTATCACCTGGTTGCCCCTTAGGTATCGCGGTCCGATCTGCGG
TGTGGTTAGGGTTTCCGAGTTTCGTGGACGACATCTCTGCCGTGAACAAC
ACAGATGTGTGGCCTGCCGTCTTCTGCGTGGGGAGTGCAGTAAGTGTGGC
TTCCCTGGTCACCCTCCCCACCCCTTCACTCCCCTCTGTTACACTGAGTC
CTCAAAGTATGGTCCTGACCCTCCCACCAACCCAGTGAGGTTTTTAGAAA
AGCTCAGAGACCCCCACCCTTAGCCCACTGACCAGGGACCCTGTGGGAGG
AAGGCCTGTCTGTGATTTAACAAACCCTCCAGGGGTCTCTGGAGCTTCCC
AGGTGGCTCACAGGTGAAGAACCCGCCTGCTAATGCAGGAGGTGCTGGTT
TGATCCCTGGGTAGGGAAGATCTCCTGGAGGAAATGGCAACCCACTCCAG
TATTTTTTGCCTGGAGGATTCCATGGACAGAGGACCCTGGGGGGCTACAG
TCCACAGGGTCGCAAAGAATCGAACACCATTGAAGGCACTTAGCATTGCA
GTACATCCCTGCCTTGCTCCCCCTGGGAAGGTCCTTCCTGCGCATCCTCT
TTCGTCCTCAGCAGAAGCGCATCTGGTTCAAAGTGCCTGGCCAGGCCTTT
CATCTCTGAGCCCGTCATCTCTCTGCCTACACATGTGAGCATCAGGAGGG
GCTTGCTGGACCAGTCTGGCTGAGCAGACCGGGAACCAAGGCACCCGCCT
GCCCCCAGGCCACGTCCCAGCCTAGGCTTCCAAGGGTGGCCACGTGTCTG
CCGAGGGCTTTCAGGGACACGAGATGACATCAGGGGTGGGGCGGGGCCAC
CTCCAGCCTGCACCTAGGGGCTGGGATGCCATTCTTCTGATTATAGCTCT
GGATCCAGCTGCTGTACAGTGCCTGCTTCTGGTGGTGGTTCTGCTACGCA
GTGGATGCCTACCTGGTGATCCAGAGGTCGGCTGGACAGAGGTATTCAGT
GCGAGCGGCCGGGTTCTTCTGCTCTAGCTCATTGCCGGAGGGGAAGGTTG
GAGAGGCCACGAGCACTTATTTCTGTAGGGTAGACACGGAAAGTGAAAGA
GGAGAGCGAAAAAGTTGGCTTAAAGCTCAACATTCAGAAAACTGAGATCA
TGGCATCCGGTCCCATCACTTCATGGCAAATAGATGGGAAAACAGTGGTA
GACTTTTTTTGGGGGGGGCTCCAAAATCACTGCAGATGGTGACTGCAGCC
ATGAAATTAAAAGATGCTTACTTCTTGGGAGAAAAGTTATGACCAACCTA
GAGAGCATATTAAAAAGCAGAGACGTTACTTTGCCAACAAAGGTCTGTCT
AGTGAAGGCTGTAGTTTTCCCAGTAGTCATGTATGGATGTGAGAGTTGGA
CTATAAAGAAAGCTGAGTGCCAAGGAATTGATGCTTTTGAACTGTGGTGT
TGGAGAAGACTCCTGAGAGTCCCTTGGACATCAGGGAGATCCAACCAGTC
CATCCTAAAGGAGATCAGTCCTGAATATTCATTGGAAGAACTGATGTTGA
AGCTGAAACTCCAGTCCTTTGGGCACCTGATGCAAAGAGCTGACTCATTG
GAAAAGACCCTGATACTGGGAAAGATTGAAGGCAGGAGGAGAAGGGGACG
ATAGAGGATGAGATGGTTGGACGGCATCACCGACTCAATGGACATGAGTT
TGAGCAAGCGGGAGTTGGTGATGGACAGGGAGGCCTGGCATGCTGCAGTC
CATGGGATCTCAGAGTTGGACACGACTGAGTGACTGAACTGAACTGAGAC
ACAGTCCATCAGAACCTCCCAGTGGGCACTCCACCAAGAAATGTCTAATG
CCAGCTCGTTTTTATGTAAAAGGCAGAAGGCACAATCAAAACAGCAAGGT
ATTCTTGCTGGGCACCTGAAACATTGTAAGTCAACTATATATATATATGT
TATATTATATATATATATATATATTAAGAAAAAATAGCAAAATATTCTCC
AGGTGCCCCCCCAACCAAAATTGAGGATTTTAAAACCATGGAAATAAGTA
ATTCAGTAAATTTCGTATTTGGAACTGGACAGATGCTAAAAAAGGATTGG
CATGATTATAAAAATTCTGATGGTATCTAAAGGTATAGGAAGAAGTTCTA
AGAAGTGTGTATGGATGTGTTATGCTAAGTACAGAGCCCAAGATTTCTGG
TGCTGGAGAGGGGGCTGTCTAGGAGGAATTACACAGTGGTTAGTGCCTTG
GCCCCAGTGGGTTCAAGTTCCTGACCCTCACTCACTCACCTCAGTCACTG
AGTGACTGTTGGCAAGTTTCTGAACCTCTCTGGATCACCTGCAAGGCTTA
CTGACTCTGCTCCTTTGGGGGGACAGGGTGGCGTGTGTGGGTGTACAGAG
ATGAAGGATGGGGATGGTTAAGGATGTAAGGATGGAGGTGGTAAGAGTGT
AAACTCTGCAAGGAATGGTCAAGGGTGGCAGTGGAGAGAGATAGGAAGTC
TCTCAGCTCTCCTGGAATGTCAGATGGCCACCCACTTCTCAGAGACCTTC
TTTGTGGTTTGGACTGAGCATTAAGACCCTTGCATGTTGGGTACCCAGAT
GACTTCAGGAAGGACAGAGGCTGGGAACCTCTTGCTACTGGGGGAGCTAG
GTCCCTAGGAGGAATTATGCCAGCTCATTTTTATAAAAGGAAGAAGGCAC
AATCAAAACAGCAAGGTATTCTTGCTGAGCACCTGAAATGTCCATCCCTT
CTGTCAATCTTGAGAGCCTTCAGTTCAGTTCAGTCGCTCAGTTGTGTCCG
ACTCTTTGTGACCCCTGCAGCACGCCAGGCCTCCGTGTCCATCACCAACT
CCCGGAGTTGACTCAAACTCATGTCCATTGAGTCAGTGATGCCATCCAGC
CATCTCATCCTCTGTTGTCCCCTTCTCCTCCTGCCTTCAATCTTTCCCAG
CATCAGGGTCTTTTCCAATGAGTCAGCTCTTCGCATCAGGTGGCCAAAGG
ATTGGAGTTTCAGCTTCAGCTTCAGTCCTTCCAAAGGATATTCAAGACTG
ATTTCTTTTAGGATGGACTGGTTGGTTCTCCTGGCAGTCCAAGGGACTCT
CAAGAGTCTTCTCCAATGCCACAGTTCCAAAGCATCAATTCTTCAGTGCT
CAGCTTTCTTTATGGTCCGACTTGCACATCCATACATAACTACTGGAAAC
ACCATAGCTTTGCCTAGATGGACCTTGAAAGCCTTAGGTTAAGGGAAACA
AACCAGACACAGAAGGCCACATTTTTTACACTTCCACTTGGATGAAATGT
CCAGAATAGGCGAATCCATAGAGAGAGATGGTAGCTGAGTGGTTGCCAGG
GGCTGGGGGTGCAGGCGTGATGGGGGGAGACCCCCGAATGGGTGTGGGTG
TCCTTTTATGGGGATGAACATGTTTTCAAACGGGCCTGAGGTGGGGCTTG
CATGGCACTGTGGATATCCTAAATGCCGCTGAGTTGTTCACTTTAGAATG
GTGACTTTGATGTTACATGACTTTCACTACTCAGCACTAAGGGGTGACAA
TACATACACAAGTGGGCACAGCTGTGTGCCAATAAAACTTTATTTACAAA
AGCAGGCAGAGGGCCGGATCTGTACGGAGGGCTGTGTTTAAACAAATAAA
CAAACAAAACAACCTCAGAGGCACAATCAGGGGGCTACTTGCTGAGATTC
TGCTCTGCATGTGTTTTACCGCTGCTCGCAGAGTGTGGGGCCGGGCAGGC
GCTGGACCTGAGGTTCTGGACCTGTATTCTCTCTCCCCCAGCACCATCCT
GCTGTACCACCTCATGACCTGGGGCCTGGCTGCCCTGCTGAGCGTGGAGG
GTGCCCTCATGCTGTACTATCCTTCCATGGCCAGGTAAGCCGGGGCTCCC
CCAATGCCTGACCCCCGTGGGGCGTCCTCCTGCAGACATGCCTGGGTGCT
GACCCCATGTGGGTGGGCGGGCCTTCGTCCAGGATCCGTGCAGCTGCGAG
CACGTGCCTGGCTCCTTGAGCCAGCCAGGGTCCCCTGCTCCTGCCCATCA
CGTGTGTTGGGTTGGTGCTGTGCCCAGTGTGGATACAGAGCTTGCAGGGC
ACCATGGGTGTCCTAAATGCTGCTGAGTTGTTCACTTTACAATGGTGACT
TTGATGTTATGTGACTTTCCCTACTCAGCACTAATGGGTAAAGCAGCCAT
AGACAATACATACACAAGTGGGCATGGCTGTGTGCCAATAAAACTTTATT
GACAAAAACAGGCGGAGGGTGGGATCTGTACTGAGGGCTGTGTTTAAACA
AACAAACAAAAAAAACAACCTCAGAGGCACAATCGGAGCTGGGGGTGCAT
CTCTCCTCCCTACACACCTGTGATGGCTCTCTCCTACCTGCTGTTGATTT
TTTTTTAATCCTTCAAACCAGCTTTCTCTGGGCACGCAGGTGAGTTGAAC
ATCCCAGGGCTGAGTTCCAGATGTCTGGGAGACTCAAAGGGTTGCTTTTA
GAATAGCTCATTTTCAGCCTCTGCTGTCTGCACTTTTTTTTCATGTCTCC
ATCCTGTGCTCTTCTGAACACACGCTGGGAGATGTGGCCTCTGAAACTGT
CTCAGACATTTCTTTGAGTCTGAACCCCTGCCCTTTACCATTTGTAAAAC
CTCAAGGTCTCTCTAAGGAGATATATTCTACATTTTAATGGAAAACTGAA
AATCAGACGTTGGACACCATCTCTTGGTGATTTTAAGGTGTTGTAGACCC
AGGGAGGGGCTTCCTGGGTAGCTCAGCTGGTAAAGAGTCTGCGTGCAATG
CAGGAGACTCCAGTTCAATTCCTGGGTTGGGAAGTTCCCCCAGAGAAGGG
ATAGGCTACCCACTCCAGTATTCTTGGACTTCCCTGGTGGTTCAGACAGT
AAAGAATCCGCCTGCAGTGTGGGAGACCTGGGTTCAATTCCTGGGTTGGG
AAGATCCCCTGGAGGAGGGCATGGCAACCCACTCCAGTATTCTTGCCTGG
AGAATCCCATGGACAGAGGAGCCTGGCGGGCCACAGTCCATGGGGTTGCA
AAGAGTTGGACACAAATGAGCAACTAAGCACAGCACAGCACAGACCCAAG
GATCAGTACAGCATGGCCCATAGTACAGATCCGGCCCTTTGCCTGCTTTT
GTAAATAAAGTTTTATTGGCACACAGCCATACCCATTTGTGTATGTATTG
TCTATGGCTGCTTTATCCATTAGAGTTGAGTAGATTTGACAGAGGCCATG
AGGGTTCTGAAGCCTAAAATATTTCCTATCTGTTCCTTTAGAGAAAAAGT
TTGCTGACCTGTGGTGTAGACTGTTTCTCCCCATCTTTCTTAATTTCTTG
CAGCTGCTCACTCAAATGTCTCCACAGTCCTATTGCTTTGTCAACTGTCA
CTACTTCCAGGCCTCTGTGTAGACCCCTCAAAATACTCTGCTCTCCCCAG
GAGCCTTAAAAGGACGAAGACAGAGTAAGCAGCTGTTACAGAGGGATGCA
CTTAATCGGAAAGAGAATTTTTATCACTCACCTCCACCCATGGGATCCTG
GGGCTGTTTTCATTTGAGCCCAGGTGCTCCCAGATTAACACAAGGCCTCC
ACCTGAAGCATTTTCATTCTCCATCATCTTATCCTCCAACATCTCCACCC
TCAATCATCCCCACCCTCTATCATCTCCAATATGCATCACCTCCAACCTC
CATCATCTCCATCCACCATCATCCCATCATGCCCATCTTCCATCATCTCA
TAGGCCATCATCTCACCCTCGAACATCTGCACCCAATATCATCCCCACCC
TCCAATATCTCCACCCTCAATCATCCCCACCCTCCAACTTCTGCAACATC
ACATGCAACCATCATCATCTGCAACTTCCATCATCCTATCTTCTCTAACT
TCCATCATCTCATCCTCTATCATCTCATCATCTCGTCTTCCATCATCTCT
ACTCTCCATCATCTCCACCTTCCATCATCTCATCTTTCATCATCTCCACC
ATCCATCATCCCACCCTCCATCATCTCCACCATCCATCATTTTCATCCTC
CATCAGAACCGTCCTCCATAATCTCCAGCCACCATCATCTCATCTGCCTC
ATGTCCACCCTCCACCATCTCCACCCTCCATCAGTCTGCCCTCCACCATC
CCATCATCTCCACCCTCCATCATCCCATCATGTCTACCTCCATCTCCTCC
TCCTCCATCATCTCCTTCTCCATCATTGCCACATTCCATCATCTCCACCT
TCCATCATCCCATCATGCCCACCCTGCATCATCTCATCCTCCAGTGTCTT
CACCCTCCATCATCTCCACCCTCCATCATCCCATCATCTTCTCTGCACCT
TCCATTGTCTCCACCCTTCATCATTTGCATCCTCAGAGCACTGTGCAGGC
TCCCACCCTCACTCATCTGTCCCTCATCATTCGAATCTCATCTTTTGCTC
CTCCTGTTTCCCTTTCTCCCCTCAAGCCCTTGTAGACCATGGTTTAGCTT
TCACAGCTGAAGAAGAAAGCTTCATTTATAAAAGAACGCACCATCTTCAC
ATGGTATTGCCATCCTTAGGGCAACAAAAGGTGGGCACTGCATTGTTCAT
GCCTGGTTTTGTGCATTCTTCCACTTTCATCTCTGGTGTGTGCACTCCAT
ACTGATGACAGCACCAGCATCTTTATCATGCCCCCTTCCCGGGCCTCCAG
GGCTACGCCCTCCTCTGGCTTTCCCCCTCATCTGGGGTTGGACCTGAGCA
ACTTCTCCTTTGGATGCCCTCTCCCCCTTTGCCACTTTCTCTACATTATG
CATTTGTTCAGTGACACTCCCGGCCTTGGTAACATAGCTGTGTGTAGCAT
ACCACCATGTCCCGTTGCTGACGGACGAGTCCCCCAGCCCATCAGTAGAC
ATTCCTGTCTTTGTTTCTGAAAAGAACCACCGAAATGATTATGGGTCACC
TTGGGAACAAGTGTCCTGAGAAATAGGAAGCTCGGGTGTTTTCAGTTGTT
CGAGGGCAGAGCTTGGATCTTATTGGTGATGTCAGCCAGGCCTAGAACGT
AGTAAAGTGCTGAGCACCTTACCAGTCTATTGAGTGCCTTTGTCTTGCTT
GAACTGTGTGTGTTTTCTTTGAAGGGGTCCCACAGGGGGGTTGTGGTGGG
GGTGGGCGTCCATCATCCCAGAGTCTTTCCTGTCTTTACTTTTTTCCAAG
GGGGAACTTGAAAAGTGGCCCACTTTCTGAGACAAGACAGAACTGTGATT
GATCTTCCTTCTCCTTCTGTGACTTACTTGCTTGTCCACAGACATGATGA
GATGTGAGTCAGGTTTCCTTCCGGCAGCCAGAAGTGATCTGTGCATTTCC
CTTTGCGCTCTGGGTCCTCTTATCTCGGCTTTCAGATAAGGCAGCGGGTT
ATTTCAGGTAAAGCCTTATTGTTGGAGAATGCCTCGGTTTTTCTCTCCCC
CCCTCCCCACCAGGTGCGAGAGGGGCCTGGAGCATGCCATCCCCCACTAC
ATCACCACGTACTTGCCGCTGCTACTGGTCCTGGTGGGCAACCCCATCCT
ATTTCGAAAGACAGTGACCGCAGGTAAAGGGCAGGGAAGCTTCCTGGGGA
TGAGGCCACGGGGATGAGCCCCAGGACTTGGGCCCCAGTCATTACCTAGG
ATCCCTGACCTCATCTGTCATCCTGCAGAGACGAGCAGAATGAAGTTGGG
GAGAGTGAGTGGGTTTACATTAGTGACAGGGAGCAAGCCTACTCAGTCAC
GGCTAGATGGTCTTTGCCAGCATGCGTTAGCGCACAGCCCAGGGACCTGT
GGACTTTTTCCCTTCTACCTGGACTCATCCTGGGTCTATCCGCGGCGTGC
CCCCAAAGACCCAGTGTCCTTGAAAACTGGCGGAGGGGGAGATGGGAACA
GGAGAGGAAAGGAAAGGTCAGCCGTGGGCGAGGGGTGGAGATGGACAGGG
CTGTCCCCGGTGGCGGAGGGGCTGGCTCCCCCCGTGCTGTCTGGGCCCAT
GTCTTCTCCCAGCTGGGCAGGAAGCAGGAAGGGGCCCACAGGACATGAGT
CAGAGGCACCAGAAAGATCTGGGCGAGAAGGTGAGTCAGCAGACCCACAG
GAGGGGGCCTGCATTCCAGCCCCCTACCCCATCCTTGAGGAGACGAGCCC
TGGGGCAATTCTCTAGCCCCCTCGTGCCCTGTGGGTCCCTCACGGGCAGA
ATTGGAATAAAGAAACACACCTCCACAGAAAGGTCACTGGAGGATCGACT
GTGGATTGCAGAACTTTAAGCGACATGCTCTGCTACTTAGGGAGACAACA
TCACGAAGGCTCTTTGGGTCTCTCTCTCTTCGTATCTGCGTTGAAAAACA
GTGTGTGTACACCTGTCAGTAACACAACATTGGAAATCGACTCTACTGCA
ATAAAAATTTAAAAACACGCAACCCCCATAGTGTATTAGAAAGAAAGAAA
GTGAAGTCACTCAGTCGTGTCAGACTCTTTGTGACCCCATGGGTTGTAGC
CTACCAGGCTTCTCCATCCATGGAATTTTCTAGGCAAGAGTACCGTAGTG
GGTTGCTATTTCCTTTTCCAGGGGATCTTCCCAACCCAGGGATTGAACCC
TGGTCTCCCGCATTTTAGGCAGACGCTTTTACCATCTGAGCCAAATACAG
CTTTTTAAAAAACATTGCATTAAAAACAGTGATCCAATTACATCAAATAA
AGGAAAACGAAGTGTAGGAAAAAACACTGGAAAGAAATATACTAAGATGC
TAACAGTAGTTGTCTTTGGGGGGATGATAGGTTATTAATTTTTTTTCTAA
ACTTTTTTTTATATTTTTCAACTTGTCTAAAGGAGGATGCCAAATCTTTC
AATATTTTAAAATAGCAAGTATGAGACTCCCCTGGAGGTCCAAGGGTTAG
GACTACACGCTTTCACTGCAGGGGGGCCTGGGTTTAATCCCTAGTTGGGG
AACTAAGATCCCACGTGCAGAGAGTCTTGGCCAAAGAAAGAAAGAAATCA
AGGATAAATTATTACTGTTTACATTTTTTGGCCACATCTTTCAGCATGGG
GCATTATAGTTTCCTGATCAGAGATTGAACCTTGCCCCCTGTGTTGGCAT
GGAATCTTATAACCACTGAACCACGAGAGAAGTCCCCTAGTGAATTATTT
TTATAATAAAAATAACATTTATATTTATATTTAATATTTATAAAATAATT
TTTATTATAAAGATAAAAAGTATAAACTGTTAAAAATACAGCTCCTGAAC
GAATAAAATCCAGATAAAATCAGGGGTGTAGTTAACAGCACTGCACCAGC
GTTATTTTCTTCATGCTGACCAACGTGTGGCTGTGAGTGTGGTTGCGGAA
GACGTTAACACTGGAGGCAGCTGGGTGCAGCTATATGGGGGCTCTACTAT
CTCTGTATATCTTATATAAATCTAAACAATTTCAAAAATGAAAGTGAAAG
TGGCTCAGTCATGTCGTGAACCCATGGACTATAAAGTCCGAGGAATTCTC
CAGGCCAGAATACTGGAGTGGGTAGCCTTTCCCTTCTCCAGGGGGATCTT
CCCAAGCCCAGGGACCGAACCCATGTCTACCGCATTGCGGGCAAATTTAC
CAGCTGAGCCACCAGAGAAGCCCCAGAATCCTGGAGTGGGTAGCCTCTTC
CTTCTCCAGGGGCTCTTCCTGACCCAGGAATCGAACTGGGGTCTCCTGCA
TTGCAGGCGGATTCTTTACCAGCTGAGCTATCAGGGAAGCCACAAAGACT
TCAAAGGAAACCTTTAAGAGCTGTGTATTCCCCCCGAGTGTCTGAGAAGC
ACCCCCGCCCCTCCCCATGCAGTGGTGAGAGAGGCATGGCTGGTATCTTG
CCTCCTGCTCTCCAGGGTGACTTGCGGGCTCCCATCCGGTTTGCTGGCCT
TTGTCGATCGTGAACATCACACACCGTTCCCCTCTGCTCCACAGTGGCCT
CCTTACTGAAGGGAAGACAAGGCATTTACACGGAGAACGAGAGACGCATG
GGAGCCAGGATCAAGACCCGATTCTTCAAAATAATGCTGGTTTTCATTGT
TTGGTAACGTGTTTCTGTTTCTTTAAACTCTCACTGGGACGTGAAGTGCA
AACCTCTTGTAAGTTGCCTTCCCAAGTGTGACCTGAGTTCCAGAGGCAGA
GTCTTGGGAGAGGAAACCTTTAGAGGCTTGTCCAGATGGCCCTGGAGGCA
ATGCTGTCCAGACAGGAAGGCCAGTCTGCACCCATGACCTTCTGGAAAGA
AATTCACATCCGCTCAAATTATAGAGAATCGCTAAGAACAGAAAATCCTC
ACTTTAACGTGGTCACATCCCAACCACTTGCCTCTGGAGTACTGTCCCAT
CGCCGAGGGTTAACATTCAAATCAAATGCTTCATCGTGCCTTCTAAATCC
ATCTTTTATTTTTGCACCCATCTTTGTTTTATTTTTTAATTAATTAGTTT
TTTGTTGTTGTTGCTGCAGTGTGAGGCATGTGGGATCTTACTTCCCCGGC
CAGGGATTGAACCCTCATCCCTCTTGCACTGGAAGTACCGAGTCCTGACC
ACTGGATGGCCAGAGAAGTCCCTGCATTTGTCTTGACAGCAGGATTCCTG
TATCTGATGAGACTCAGGGATGACACCCCGTGTCTGTGTGATGACTCACA
GCTCAGGGCAACAAGGACAGAAACCATGACTCCCCAGGCAGTCCAGTGGT
TAGGACTCTAAGCTCCCACTGCAGGGGGCAAGAGTTCTATCTCTGGTTCA
GGAAACTAAGATCCCGAAAGCTGTGTGGCACGGCCAAAAAAACAACTAAA
AAAAAAAAAAACAAAAAAACAAGAAGGGTAGAGAGCAATCTTCATCTCCT
CAGTGGGCCTGCCCTGTCCCCCACAGGCAGTCTGCCCCAGCATTTCACGT
GGGTTTGCTCTGTGCAAATAGAGCTGTGACAAGTACCAAATCAGAAAGCC
CTTTGCTGCAGCGACAGAGTGCTTTGCCAGACAGGGCACACGTTGGTGGT
TAATAATTTATCTGATGGGAAGAGGCTGGTTTGGACATTCTCCTGGGAGA
GCCGTGAGTCACCTCCCTTTAAAAATACCCCGAGCCTGCCATTTCCAACT
TCGCCGACCTGCCGACACTACCGTCAGAAATTTCCGACATAAAACATCAA
GGTTCGTGTTGGTTTCCATCAAGACGTCAGCTTGCTCGTCAAAGGAGGAA
CGACTTCTGGGCTATTTGACGTGTGCCAGAACCTTCCACCCGATGTGGTG
TTGATTGTGACCATCGGGAGGGTCTCTTGGTCTCTGCTTTTGGCTGAGGG
GGATGCCGTCTCTGACGTCTAATTCTCGTCTAATTCTGCAGCTACTTGGT
CACCCCTCCCGGACTTCTAGGTCTTGGGAGACATGTTCCCTGGGGCTTCT
CAGGTGGCTCAGTAGATAAAAAAATCTGCCTGCAATGCCAGAGATGTGGG
TTTGATCCCTAAGTTGGGAAGATCCCCTGGAGGAGGAAATGGCAACCCAC
TCCAGTATTCTTGCCTGGGAAATCCCATGGACAGAGGAGCCTGGAGAGCT
ACAGTCCATTGGGGTCACAAAGAGTTAGACATGATGGAGTGACTCAGCAG
TCATGCATGCAGACATGTCCTCTGACCTCTGGCATTGAAAAAAAAGGATA
TTGGGAAGCAATTATCCTCCCATTAAAAATAAGCAAAAAAAAAAAAGAAT
GATATCACATGTTCTCTTTACCTGTGGCCCATTTTCTTTCTCACAGCTGG
TTCTCAAATGTCATCAACGAAAGCCTTTTGTTCTATCTTGAAATGCAACC
AGATATCAACAGCAGCTCTTTGAAACAGGTCAGAAACGCAGCCAAGACCA
CGTGGTTCATGATGGTAGGTCAATCTTGATTTTAAATGTACTTCCTAAAG
GAGAAGAAAAAAACCCAGACAAGATAAAAACCACTGAGAAGTATGATCTA
TGTGTTATTTAAATATGTGGTGAGTTGAAGAGGTTGCGGGGGCCCCCTTA
TCTGGCTGTGGTCCATGAGTTAGCTTGTGCATTTACTCTTGGCATCAGTA
TTAAAGGGATCCGACTCTGGTAAGTCAGTTTGGTAAGTGTCTCAAACCTT
TCAGGCTGACTAAACATATCTGGGAATTTATCTGCAAGAAATGTTTCAGA
CTGAAAAAGCCACAAAGTTGGAGGTATTCCTTGGAACACTATCCATAAAT
ATAAAGCATTCTAGACAACCCACATTTTCTTTTTTTTATAAAAGGGAATG
GTTAAGTAAATCACTGTGTTTGATCTTGATGGAATGGTATGCAGATGCTG
AAAATGACAATGTAAAGTATGACAATTATGAAATCTCATAATAAGTGAAA
AATGCAGGATGTAGAGATTATACTCATTTCAACTACCTAATCACACATGT
AGGCAACAATATTGCCTAGAAACACACCACCATAATCAGGTGGAAAGCTC
ATGGATACATCGTCCTCAATGTTTCCAGGATATTGTGTCCCCACTGCAAA
AACTCCATACAGGAGCTGTTAGTGGAGAAGTGCCTGGGTTTCCGTGGTGG
CTCAGCGGTAAAGAATCCGCCTGCCAATGAAGGAGACCCCAGTTCAATCC
CTGGGTCAGGAAGATCCCCTGGAGAAAGGCCTGGCAACCCACCACTTAGC
AAGTAAACAAGTGGAGAGGCTCACCGTGTCCTCAATTAGATGCGTGTGCA
TTCAGTGTTGCCATCATTATTAAACACATATGACTCTGGTAAGTTAGTTT
GGCAAGTGTCTCAAACTTTTTAGGCAGACTAAGAATATCTGGGAATTTCT
CTGGAAGAAGTGTTGCAGACTGCAAAAGCCACAGAGTTGGAGGTATCCCT
TGGAACACTGTCCATAAATATAAAGCATTCTAGAGAACCCACATTGCTGG
GGTGATGCCCGGGACCTTCCTCTCCACCCCTCTTGTCTCCTATGTCTCCC
ACACATCTCTGAAGGCTCAGAACCTCCAAGCCAGTCTCTCTCTTGCCCCA
AACTAGCCAACAGGGTGCACCCCAGTTGAGGCCAGCCCTGTGGACCGAAG
TGACCCCTGTAAGCCGTGATGCCCATCAGTGCCCTGACTTATATGCCGTC
TCTTTCCCCTCCCTTCCAGGGGATCCTGAATCCAGCCCAAGGTTTCCTGT
TGTCCCTGGCCTTCTATGGCTGGACGGGCTGCCGCCTGACGCTTCCAGGT
CCCAGCAAGGAGATCCAGTGGGACTCGATGACCACCTCGGCCACCGAGGG
GGCGCCCCCCTCCCCCGGGGGCCCCCAAGAGCCCGGGGAAGGCCCCGCTC
CCAAGAAGGAGCTTCCGGGCGGCACGCACACTTCCGATGAGGCCTTGAGC
TTGCTTTCTGAAGGTAAGAGCCTCTGGGTAGAGGCAGGCCTAGGTTGGGG
GCCCCACGCTCCCCAGGAACTCCTTTGTCAATGACCAGGAGGCTCCCTGC
CCTGATTTCTGGAACCAGCTTCAGGGCATGAGTGGAAAGGGACCACCTTA
ATCTTGCAGCTCATCACTCACCTCTCCTTGTAATGTGAGCGTGGTGCCTT
TCCTCTGCCTGCGTGCCACGTGGCTTCAGTCCTGTTGAACTCTCTGTGTC
CCCAGGGACTGTCACCGCGGCTAGGCTCCTCTGTCCCTGGGGATTCTCCA
GGCTAGAATACTGGAGCGGGTTGCCATGCCCTCCTCCAGGGGATCTTCCC
CACCCAGGGACTGAGCACCGTAGCCTTGTGTTAATAATCACTAGCTCTGC
CTTGGGCTGCATGACATTCTGAGTCTGAGATAAACGGTCACTTCTCATCC
GGTGCTGACGTGTGCTGTAAGACAATGAACAGTAGGCGACCTGACCTCTG
GAGATTTTTACGTGCAGTTTTGGTTTGTCCACTTCATCCGGGGGCATTTA
AGATGAAATGCAGGTCACCCGTCGCTGGCTCTTCCACTCTGTCCTCTTCA
AGACTCTCGCACCGTGGTCCTCAAATTGGTCCTCTTTTTAAACTCCTGTT
CATCTAATTTGGACTTCCCCTGGTGGCTCAGATGGTAAAGAGTCTGCCTA
CAATGCTGGAGACCTGGGTTCGATCCCTGAGTCAGGAAGATTCCCCTGGA
GAAGGAAACGGCAACCCATTCCAACCTTCTGCCTGGAGAATCTCATGGAC
GGAGGAGTCTGGTGGGCTACAGTCCGTGGGGTCGCAGAGTCGGACACCAC
TGAGAGACTTCACTTTCACTTTTCATCTAATCTGGGGTTATGTCCATTAA
GCCAACTGATGTGGAGGGTGCAAGTATTAGTTTAAGGCCCAAATAATGTT
TCTTTAAAGCTTGAAAGTGAAAGTGAAGTCAAGACTGCTTTCCTTTAGGA
TGGACTGGTTGGATCTCCTTGCAGTCCAAGGGACTCTCAAGGGTCTTCTC
TAACACCACAGTTAAAGATGTGGGTGAAAATCAGCAAAGAGAATGACTCC
AATATACAAGACCCCTATGTCTATACCACAGGCTGTATGGTGAGGCCAGA
ACCCTCAGGAACCGGGGGCCTTTTCGAATAAAGTGGTAATCTCGGAAATG
AGATAAGGCTGCCTTAATCTCGGGCGTGTTTCCTTTCTCCTTTCCAGCCA
CTTTGCTTTCATAAGACAGAAAATACTGTTACATGTCAGAGAAAAACCCA
GAGATACAAACTCGAAGTGGTAAGTACGGTTAGGCCTCTCCCCGTCTGCA
CTGTTAGGATGAAGCTTGACCTGTTTATCATATCCTTAAGCCTCTATCTG
CTACTTTAGGATTGACTTCCAGGCTGACAGGCTTCCACCGAGCCTTCGTC
ATTAAGCATCTAACATGGTGTCTGGCACTTACTCGCCACTTAACAGAAAA
GGCAACTGAATTTGAACAAATATTCCTTGAGCACCTGCAACCCAATCAAC
ACTACTTTAGCTGAGGCGGGGGCTGGGGTGTGAGCGTCTCTGAGAAGCAT
AATGTGGGCAACTTAGAAATAAAGACTTATAAATAAATAAAGGCTGAATT
TGCACGTAGTTTGGTGGTGGGAAGCTGGAGAGAGGCATGTGGGGAAGAGA
TCTATCCAGGGGTGTTTACAGATTCTGCATTTTTATCTTGGGGCCAGCAA
AAGGCCAGTTAAGGTTTCTGAGCGGAGATAGAACACTTACATTTGCTCGT
AGGACTAACCTAGTGACTGGAAAATTCCATGGATGGAGGAGCCTAGTGGG
GCTACAGTCCATGGGTTCACAAAGAGTCAGACATAACTGAGCGACTTCAC
TTTCATGACTATTGCGGCGGCTCACAGTGAGAGGATCTGTGTATAATTCC
TGATGACAACTTGAGTTCAAAATACGAGGAGCATCTTTGTAAAGAATCAG
GATTTCCCCCAAGCCCTCACATCTTTCCAATTATCCCTTGCTTTTCCAGT
CTTCCTCCTTCTCCATGCAATCCCGTGTTTCCCACAAAGCTCTATTTCCC
TGTAATTCCCTACTTCTCTCTCCAACCCCATGTCTTTTCCAACTCTACAC
TTCTCCCACAATTTTCTTTCTTCCCCATAATCCTTGCCCTGCCCACAACT
CTCCATTTTCCCACAGCTTTCTGTTTCATTCCAGAATTCCTCTTTTGAAT
AATTTTACTTTCCCACAGCTCTTTGCTGTCTGTGGTCAGTCTCATATCAC
CATTTTATCTCCTAGGGTTCTGTTCCCGACCACGTTTCAGTTCTTCTGCC
ATTTTATTCTTCTATGATTTTCTGTTCTCAATTTTGCTTTTTTCTTTTCC
ACAACTGCCTATTCATCCCCAAATTATTCATTCTTATGCAAAAATCTCCC
AGGGCTCCCCTAACCCTCTTTTTCATGACGACCAGTCTCTCCCACAACAC
GCTTTCCCCGTAACGAAACAACGTAAGACTGTATGATCCTTGCCCACTGC
CGCCCGCCATTCCGTCATGTTCTCTGCCTGCCTGCTGTGGGTGGAGTTTA
GTCAAACAATCAGTTTAATCACAGAGGTGAGAAATGCTAAAACAAAGGAA
AACAGTCAAAGGAGACCAAATAATAATAATGTACTCATTAAGTGTAGTGA
GATTTCCACTGTAGCTCAAACAGCAAAGAATCTGCCTGTGAGGCAGGAAA
CCCGGGTTTGATCCCTGGGTCGGGAAGATCCCCTGGAGAACAGAATGGCA
ACCCACTCCAGTATTATTGCCTGGAGAATTCCATGGACAGAGGAGCCTGG
TGGGCTACAGTCCATGGGGTCACAAAGAGACATGACTGAGTGACGAACAC
ACAGACACCAGGACCTTTAGTTCTTTCTGAAGGGGCTATAGATAATATTC
TGAGCCACCTCCTGTGAGCTGTCTTATAGATACCAGAACACACCAGGTGG
AAATTAACGACATGATGACCCAACTGTACCCAGGACTTGCTGCTGCTGCT
GCTGCTGCTGTCGCTTCAGTCGTGTCCGACTCTGTGCGACCCCATAGACG
GCAGCCCACCATGCTCCCCTGTCCCTGGGATTCTCCAGGCAAGAACACTG
GAGTGGGTTGCCATTTCCTCCCACAATGCATGAAAGTGAAAAGTGAAAGT
GAAGTCGCTCAGTCGTGTCTGACTCTTAGCACCATGAAGTCAGTGAAGAG
GATTATCAGAGGTGACTGTACTGCTTCTGCGTGTAACGCGCCTCCCCCAC
CCCACCGCAACTTTGTCTCTAAAAGCTCTCACTCTCTGCCTGTCGGGGGG
GTTGGCCTTTGGACAGACGTCTGCCACCCTCCTTGTAGTTGCTGGCATCT
GAAACAAAGCAAATTTTTCCTTTCCACCAACCTGGCCCGCTTATTGGCTT
TTGAGCGGCAAGCAGCCAGACCCTTTCATGCATACCTTTTGGTCACTGTA
ATCTTCTGTTCTGACAGTCCTGTATCTCTCCCTCAAGTTCCTGTCCTTTC
CCACAGCACCCTTTCTCACGATTCTGTCTCTCCCATCTCTCTGTATCCTC
TGACACAATTCTGTGGCTCCAAAAGTTCCTTTTTCTCTCCGGGATGCCGC
GCTTTCTCACGGGTCTCTTTTCCCTTCATCATCCCTCGTCTCCCTCTTGC
AGTTTTCTCTTCTGCAATTCTGTTACTCTCACAATACCTTGTCTTTCCCT
CCACTCCCTGTTTCCTCTTTCTTTTGCCCCCACACATGGTTCTATTTTGC
ACAATCTTCCCTTGATCTTCAGAGTTCCCTGTTCTCTCCCACGCTTCCCC
ATTACCTTCCACGTTTCTATCCTGTCTCATGCATCTTGTTGTCTCAGATT
CTTTCCTGCCACCCATGTGCGGTCCACATAACTCCTGGTATGGGTTCCCA
ATGCCCAGGGAGTATGGGAGTGAACAGACCCCAGGGCCTCCAATCTGTCA
GGCTGGCTAAACCTTGAAGGCGGTTCCACAGCTTGAAGGGGATGCAGGGC
TAGGTGTCACACATTCTCTATAGCTTATGCTCCATACTTCCTGGCTGGGG
TTGCCAATTTAGGGCTTTGAAATGTCAAGGAATCCAAGGACAATGGCCCA
GGGCCCAAGAGACGACATGCCTCCCCGAATGGCAGGTCTACACGGAGCTA
GGAAGCTCCCAAGAGCCTGCTTGGACTTGCAAGGCTCACTGCCGGGAGTT
GCTAGGAAGGAGGCCTGGCCCTTGGGCACTGTGAGGTCACCAGCAGCTGC
ACTTCCTGCAAGGACACTCAGGTATTCACACGCCACGGATGCAGAATGAG
TGCACACCTACCACAGCCTTGTTGGATTTGCTAAGACCAGACGAGAAACC
CGTGGAAAGGCAGACATGCTGGAAAGCACTCGGTGCAGACAGCAGGCCAG
CTCAGCCCAGGCGGGGCTAGGCTTGGGGGTTCAGGGGTCATCTGCAATGG
TGCTGGCATCTGAGACTCACAGAATGGTTGTGGCAAGAAGCGGTCTCCTT
TGTGGAGCCCGAGGGGACAGCGAAGGATGCAAGAACTTAGGAGAAACAAA
AAACCACAGGATGTGGAGCATGCCCCAGAGTGTCAGGTCACGAGGGTGGG
TGCTGAGCTGAGACCTCGATCACGAGCCACTGTGTGACCCTCCAGGCCAT
GGTCTTTCTGCAAGAACTACAGAGACTGCACACAAGTTCCCACGAGCTCA
GGATGTGAGGGTAAACAGTGTGGGGTGACCCTGTGAGAAATCAAAGCCTT
TGTGAAGCGCCTCCAGCAGGAATGCGGTTTTGGAACGGGAAGTCATCTTC
CCAGCTGGTGCCTGGTCTCCAGTTGAGCTGTTTCAAATCCTAAAAGATGA
CGCTGTGAAAGTGCTGCACTCAATATGCCAGCAAATTTGGAAAACTCAGC
AGTGGCCAAAGGACTGGAAAAGGTCAGTTTTCATTCCAATCCCAAAGAAT
GCTCAAACTGCAGCTCAATTGCACTCATCTCACATGCTAGTAAAGTGATG
CTTAAAATTCTCCAAGCCAGGCTTCAGGAATACGTGAACTGTGAACTTCC
ACATGTTCAATCTGGTTTTAGAAAAGGCAGAGGAACCAAAGATCAAATTG
CCAACATCTGTTGGATCATCAAAAAAGCAAGAGAGTTCCAGAAAAAATCT
ACTTTTGCTTTATTGACTATGCCAAAGCCTTTAAGCGTGTGAATCATAAC
AAACTGTGGAAAATTTTTAAACAGATGGGAATACCAGACCACCTGACTTG
CCTCCTGAGAAATCTGTATGCAGGTAAGGAAGCAACATTTAGAACTGGAC
ATGGAACAACAGACTGGTTCCAAATAGGAAAAGGAGTACATCAAGGCTGT
ATATTGTCGCCCTGCTTATTTAACTTATATGCAGAGTACATCATGAGAAA
CGCTGGGCTGGATGAAGCACAAGCTAGAATCAAGATTGCCAGGAGAAATA
TCAATAACCTCAGATACACAGATGACACCACCCTTATGGCAGAAAGTGAA
AATTAACTAAAGAGCCTGTTAATGAAAGTGAAAGAGGATAGTGAAAAAGT
TTGCTTAACTCTCAACATTCAGAAAACTAAGATCATGGCATCTGGTCCCA
TCACTTCATGGCAAATAGATGGGGAAACAATGGAAACTGAGAAACTTTAT
TTATTTATTTTTTTGGCTCCAAAATAACTGCAGATTGGGACTGCAGTCCA
TGGGGTCGCAGAGTCGGACACAACTGAGCAACTGAACTGAACTGACTGCC
TGGCCTCCAGGAGGAGCGCGTTCTCTGCTCCAGCCAAACAGCTGCTCCCA
CCATCTTGACAGGCAACCTCGACTCTCCTGAGATGGGGCTGCAGGGTCCA
TGCCCGTTGACATGAGCCTTTCCCCCCTTCTCCCGTCTTCTAAATTCCTC
TGCTCTCTGTGGGCTCGTCACTCTTGGTGTGACCTACCCCGACCTGGAGC
CACCCGGACGTATCTTCATCCTGCCCTGGGAGGACCCTCCCAGCGCCTCT
CCCCATGCAGCCCACCGTGGGGTTTCATGTGGTGGTCGTGGGGATGCTAC
AAGCGCTCTTGTTGGCCTGGTGATTAAGAATTTGCTTTATGATGCAAGGA
ACACCAGTCTGACCCCTGGTCCGAGAAGATCCCACATGCCATGGGGCAAT
AAAGCCCGTGTGCCACCAACTACTCAGCCTATGAGCCACAAATACTGAAA
CCCATGCTCTGCAACAACAGAAGCCACTACAATGACAAACCCACTTACGG
CAACTAGAGAAAGCCCACGTATAGCAACAACAGGCCCAGCACAGCTACAA
AGAAATAATTTAAAATAATGCACTTAAAGCACAAGCGACTCAAAAAAAAA
AAAAAAAAAATGTTGGACGTCATCGAAATTAAAAAAATAAAGGAAAAAGG
AAGTGCTGTGAACGATGGCATATCCGTAGAGGGCAGGAGACTAGTTTTAG
CGAGGGGTCCAGGACACCTCCTTGAGAATGAAGTGACATTTGAGTTGAGA
CCTGAGTGCAGTGTGGGAGTGAGCCACGAGAACATCAGGAGAGCTTCATA
GGCAGAAAAGGGGATGTGCAAAGGCCATGTGGCAGGATTGAGCTTGGGTA
TAAAGAAACCTGTGCCTGAAGTTTGTGAGCGAGCAGAGGGTCATGACGGG
AACAGGCTGCAGCCGTCCATAGCGTGTATAGGTTGAGAGAATTTGAGATT
TGGAGGTTTTCAGCAACGGGAGTCACCTGCCTCATGCACAGTTGAAAAGG
CCACCCTTCCCTATGTCTTCCATGGCTCACACACACACACACACACACAC
ACACACACCCTAGAACCTGGCACAACAGCAGGCAAGCCTGGGTATTTCTG
CCCTGAGAGAATAATCTTCACATCTGTTCCCACAGGTTCCAGAGGCAGGT
GTGTCATTGGGATGGATTAAACGGGCCTCTAAGGGTACAGTTTTGGCGAG
TGATGCCTGGCTTTGATTCCACTGATTGCTCCCCACACCCTTCCCCCTCC
TCCAGCCACATGGCATCATCTGTGAAAAGGCCACTCTTAGTTTTACCAAG
AGAAGGCCTCCGGGTACTTCCTAGAATTGTGCAAGTGGTGGCCAGGTGAC
CATGGAGCCCCCTGCCCGAGTGACCCCCCCACCCCCCAGGACCACAGAGC
ACTGAGATATCGGGACCCCTGATTTAGCGCAAGTCCCAGGGCCCTGAAGC
TGCATGGTGTCAACAGGCCCGGTTTCCATTTGCTCTGCAGCGTCCACAGG
GTTTGGCTGAGAGTGTCTCAGGGTAAGGGCAGTGGGGTGGATACAGTCAT
GACATCAGTCAAGTTGCCCGAGATGATCCGGTAGGGTTTGCTCTGTCCTT
TGGGCTGCTTTAATCCCGCGTAGGTGGACGTATTATGTATTTTTCTTTTT
TCGCTTTTTAATACGGCCGTGCAAGTTGTGAAAGGCATTCCCGGACATTG
ACACTGACCCGTGACAGTCTTTCCTCCTCCCCAGGTTCCGGCGGCAGCAC
CATTGAAATCCACATCGCAAGCGGGTCCCGCGGCGGAAAGGCCCCCGACT
CTCTTCCGAAAGTCCAAGGAACCCCGTAGAGAGGACGAGACAGAGGGCTC
TGGACCCTGTGTGTATTTTCAGACGCGACGGTTCTCATCCCTTATGACGG
TACCCTTGCCCTTCAGTCAGCACACTGCGGGGTGTAGCGTCCCCCCCAAC
TGAATCTTCCTGCCATCACAGTTAACAGAGTGTTCCCTGGCAGCCTCTGT
GTGATGCAGAGGCCCACCGTGAGCCTGTGCTTGGAAAGGAAAGGCAGATT
CCCTTGGAGCCCAGCAGCTTGTCCGGAGTCTCCGTGGACGTTCGTTTCTC
TGATCTGGCTGTAATGTCAACGCCAGATCCAGGTCCTTGGAAGAGTTAAT
AAATAACAATAATTAAAAAAAAGAAGTAGTTGTGTCCGAGCTCCCAGCCT
GACTTGGGGTCTGTCTGAATAACACCTCTAAGCGAATGCTCTTGTGTAAG
ATAAGTGATGATGCAGGGACCGGGGACGCACCTGGGAGCAAGTTCCTCAC
CCACCACTAGGGGGCAGAGGAGGGCAGGCTTGGACCACCAGGTTAGCCTT
CGCTCGGTCCTCAGCTGTGTTGAGCGGTAGCGATTCAGTCTACGGGAAGC
TTCCATTCAGTTCCGTTCAGTGGCTCAGCCGGGTCGGATTCTTTGCAACC
CCATGGACTGCAGCACGCCAGGCCTCCCTGTCCATCACCAGCTCCCGGAG
CTTGCTCAAAGGCATGTCCATCCAGCCGGTGATGCCATCCAACCATCTCA
TCCTCTCGTCCCCTTCTCCACCTGCCCTCAATCTTTGCCAGCATCAGGGT
CTTTTCCAAGGAGTCAGTTCTTCGCATCCCGTGGCCAAAGTATTGGAGCT
TCAGTATCAGTCCTTCCAGTGCACACCCAGGACTGATCTGCTTTATGATG
GACTGGTTGGATCTTCTTGCAGTCCAGGGGACTCTCAAGAGTCTTCTCCA
ACACCACAGTTCAAAAGCATCAGTTCTTCAGCACTCAGCCTTCTTTATGG
TCCAACTCTCACATCCACACATGACTATGGGAAAAACCATAGCTTTGATT
ATACAGGCTTAGGACCTGACTCTGCTTAGATTATTGCCTGTATTTCCCTC
TTTCCATAATTTTACATATATAATAATATAAAGCTGTCCTTTGGTACCCA
TGAGAGGTTTGTTCTGGGACCCCCACGGATACCAAAATCCACGGACGCCC
TCCACATCTGCATGGATGACTGTTTACAGACTTTATGTATTATAAACATA
TATGTGTATATACATATTTGGAGACAAAAATATTGTACACACTACACACA
GGAGACACAAGCCACCTAGGTAAGTCTATGGCACAAGCACACAACGTTTC
CTCAATTCTGCTGTGTGATGCTCATTCACATCAAGATTAGGACTCTTGTC
TCACTCTGGAAAAGCTGTCCAAGCTGCTTCTGGATTTGTGCTTTTGGAGT
CCTAGGGCCACAGACAGGATTCAGGGCATCCCACAACCGTGGCCGTGGTA
CCCCAGGGCCGCACGCTCCTAAAGCTGCACTTATTACCTCACAGCCACCT
TGACCCAAAGTGTCCAACAGCAGTAAAAAGGACTTTTGAGCCTTTCTGGG
TTCTTGTCCTCTGTATCGATATCCTGTCTGCCCCAGTCATCAGAGAAGCC
CCGAGACCTCTCCTCTGAGGGCTCCCCCTGCCGAGCAAGACACAGCCCCA
ACCCCATTCTAGAGCAAAGATGCTAGCCGCTGGCTTCCCCAGGCTCCCTT
GCAGCTAGAGTGTGTCTGGTGTGGCGGATGAGACTTGTGCCCCAAGTCAA
CGTTGGAAGCTGATGCGCAAGGAAATAGGCTGTGCAGAAGCAATTCTGGG
GAGGGGTTGGCAGCTGCATGTCTGCTTTGGAAGGCAGCCTGGGTGAGCCC
TGAGAGGTCAGCCCAGTGTCTGGCATCGAGCTGTGGGCAGCACCCCAGTG
CTGTGTTACCTGCATAGGCCTGATTCTGGGCCTGGCTCTAGCCAGTTCTA
TTCGCACTGTCCCCCACACCTGGGATACCCCAGAGCTGCTCAATATACTC
CTCAGCCATTCTTTCTGGCTTACGTTAGCCAGAGTCCAGTGGCTCAGCTG
GTAAAGAATCTGCCTGCAATGCAGAAGACCTGGGTTCGATCCCTGGGTCG
GGAAGATCCTCTGGAGAAGGAAATGGCAACCCACTCCAGTATTCTTGCCT
GGAGAACTCCAGGGACAGAGGAGCCTGGCGGGCTACACACAGTCCATGGG
GTAGCAGAGTCAGACACGACTTAAGCAACTAAGTTTCACTTGCAGTTCAG
TTCAGTTGCTCAGTCATGTCTCTTTGCAACCCTATGGACTGCAGCACACC
AGGCCTCCCTGTCCATCACCAACTCCCAGGGTCCACCCAAACCCATGTAC
ATGAGTCGGTGATGCCATCCAACCATCTCTTCCTCTGTCATCCCCTTCTC
CTCCTGCCCTCAATCTTTCCCATCACCAGGGTCTTTTCCAATGAGTCAGC
TCTTTGCATGAGGTGGCCAAAGTATTGGAGTTTCAGCTTCAACATCAGTC
CTTTCAATGAATACCCAGGACTGATCTCCTTCAGGATGGACTGGTTGGAT
CTCCTTGCAGTCCAAGGGACTCTCAAGAGTCTTCTCCAACACCACAGTTC
AAAAGCATCAATTCTTCGGTGCTCAGCTTTCTTTATAGTCCAACTCTCAC
ATCCATACATGACCACTGGAAAAGCCTTGGGAATATATAGCCTTGCAAAG
TGTTTAAATGAGCCTACTCATTGCCTTATTGGAAAGATTCCCCACCTCCT
CATTCACAATCATGGTTTTGGTGGAGTTTCTCTCAGCAACTGGGCTCAAG
CATAAATGTTTCCCTTTATAGGAGCTCCCATGCTCCAGAGTATTATTAAC
TTCAAGGCTCAGGGGAAGGTAACACTTTTAACAGTTCCTGCCTGCCCCAA
TCCCTGTGGAGTGTCCCTCCCCTCAACTTCCCATGCCGCTCTGCGGGTGG
ACCTGTATGTTAACTTTGCTTACCTTGCACATGGATGAAGACACTTTGCA
GAAACAGGGAGCCAGGCTGCGGAAAAAACAACAAAAATTCCACCCAATTC
TGAGTCTTTCGTAAAGGTCTGTATCTGCTGCCCACGCCTTCCCATGCGTG
TGAACTAGTGATTTGCAGGCTCCCAACACTGACAGTGACCAACACCGCGC
CCACAGGTGCACAGAACCCTCGCCGTGAGTATGAACACCTCAAAGACCTT
TTAGGAACATCACCCAGATGTGGGCTCCTAAAAAAAGAAACCAAACAAGT
TCTCTGCTCCGTGAGTGGAAACTCAGATTCCCAAATCAAACTGGGGAAGG
GGGAGGGGGTTCTCAATGGACAAATATTTTTAGAATCACTGCAGCAGAAG
GTTCCATAGAGATGAAAGTTAGGTAAAAGCTCAGGCAATCTGGTCTCAAC
CCAGTTTCCGTCACAGCACAGAGACCTCCTTGGAGGTGTGCCTGAGCTAC
CAGCGTGTTGCTTCAGCTACGGAAGCCCGAGTTCCAACACACTCGTTTAA
CAGTGGTTAAGTCCCGCCATGCGACGTGCACCCCAGTGGGTTCGGGACAT
GGCTCACCCCAGACCCTTCAGCAGCAGGGCTCGGGAGTGCTCAGAGATTC
ACCCCAGATTCGCTGCGTGAAAGCACCCTCAGTCAGCTAGGCAAGGCTTA
CAAAAAAAAAAAAAAAGAAACAAAACCAGGCAGGTCTGCACCCCGGGCAA
CACTGGGGCAGAAGCAACAGCCCAGGTCTGGCGTGATTCCCATGCAGGGT
CAGCTTGTGGCCGGAGGACATGGGGCTCTTTGGGGTGACTCGCATGGCCA
AATGCAGCTCGTCTGGCCACCTCTGTGTCAGCTCCACACTGCCTATCACC
CGGGGGACATCGCTTCAAAGGGAACCGGTCACCGTCTGCATGTGTCTGCC
TGAACCGTGTGCTGCGCCCGGTCACCAGCATCCCTACAACTTCAGGTTCT
TCTATTATTCATCCTGCACCTCAAAACTGCCAGCGACCCAAGCCTGCCTG
CAGGAGAACATCCGTCTGCTTTTTGACTTTTCTAAAACTGCCAGCCTGAA
ACAAAGTAATGAGAATGAAACTAGTTACACACACAAAAAAAGCACACTCG
CCTTTTACATCATAACGCATCTGCAACCGCTGGTCTCTTCATCTGGTTTA
CAACGAACTGGTTGTAAAAGCAGCCCTGGCCCCCGCCCATTTCTCCCAGG
GGAAAAAAACAGACAAGAGCTTGGGGACTTGAGGCAGCGGAGTTAGCGTG
GGCCCCCCAGACGAACAAGAAAGTACACACAGAAAGTTCTCTATCAAAAT
AGAGCAATTTATTTAAGATAACCTAATATGGTCTGTTGATTCTCTCTTGG
AACAAATGAGCCATCTCTCTCACACACACACATGGTGATTCACACACACA
CACACACACACACACACACACACACGGTGATTCTCTTTCCGGGAGCGTGT
GAGGGTGGGGGTTGCAGTGGGGCATTGGTGGAGAGAACGGAACGGGGTGA
GGGGTGCCGTGTGGATGGAACAGAAGCTGGAAGATGCGGACAGGACATCT
GGCTGTACGTCTGACACAGACAATAAAAACCGCAGAAGCCAGACATTCAC
AAACCTCAGGGAGCGGCGGGAGGAAAACACTCTGCATTGGATGGGAAACA
CGATCAGAAAAAAAAAAAAAAGGCTGAAAGGTTAAAACAAAACAAAACAA
AAAGTAGACCCTTACTCCCGACAAAGTGCACTTCACTCGGCTCCGAGATC
TCTCTTCAGAAAGACAAAGTGAACATCGCGGTTCTGTAGCCGTGGAGTGG
GCTCGGACGCGCTTCCCCGGGCGTGCGTGTGTGTGTGTGTGTGTGCGTGC
GCGCGCGCGAGCGAGCAGAACGAGCTCCCCGGCAAGGTGGACAATTCCGG
GGATGGATCTGTGCGTTACTCTCTGCTTTCAGCACCAAGGTCCCATCTGC
ACACTGAGGACTACGAAGCGTTCTACACGAAGTCTAGAAGCCGCTCGCAC
TGACGAAGCGATGCGCCCACTGAGCGCTGGCAGAGGGTGCAGCGGGTGTG
ACTCAGGGCAGGCATGATTTCAGAGGCGCCGGCTGCCTCCTCCTCGTGGT
CCCCATCCCGGGGCCTCTTCCTGATGGTTCATGGGCAGTTCTGCCTTCCT
CAGCAGCTTCAGTTCATCACCACGCCCGGTATCCAGTCCGTGTCCTTGGC
TTTACAGCCCCTGGCTCCAACCCGGCAACTTCCAGCCTGGTACAGGTTTA
CCAGGAGGCCCCCGTCAATACTTGGGAGACCCTGCGTACCAACCTCCCCG
TGACGATCCTCTCGTGGGCCTTTTATATCAAATCCCACCTTCACCACCCT
CCAGCAGCATTTGGCTCATCAAATGCAAACCTTGTCTGGACCCCGGGCAG
CTGATGCTGTGTGGACAGTGACACGTTTTCATTCGGGGAGGGGTCCAGAT
GGTGTTCAACTAGACCTCTGCAAACTGCATCTCAAAGTTAATCATTTTGG
GGGGGGTGAGGGGGGTCAGTGATTATCCGTGGACGCCTGAGGGCGTCTGC
TATCTTCAAAGGTGGGAGTGGAGACGCCAATCAAACACTGACCTTCTGAA
AGTTTGAGTGTTTAGATACAGCAGAGCCTGAGAACACGCCTGACCTGTGA
AGAGTGCCTGTTGGTCTTCTGCAGTCAAGGGGGTTTGAAAAACTCTCAGA
TCCATGGAGATAGATAGTGAGCTGAGAGAGAGGAATGCACTCTGGCTTTG
GGGGTGCTCCCAGGACAATCCAACTTCTGCACAAAGGCCTAGCCATGCCT
TACCGTTGGCTTCGGGGTCCATGGACAGATACCACCACCCTCCAAAACCA
TCCAGGCTGGTTGCTGGAACACAAAAGGGCTGCCAACCTACCCACCGCCA
GCCCCATCCCGGGGGGAAGACGGATAAGCCCAGAGGCCCTGCTGCTAATT
TACAAGCTGGGTCAGCATGGGGTCAGGACAGCAGTAGAAGTACAAAGGTT
ACCTGATCCAAGGGAAAGGAAAACTCCCCTAGGGACACACAAAGCCATGT
TTTAATTTTTTTTTTTTTTTAAAAACAGTAAAAAGATTCATTGTTTAAAA
AAAAAGGGCAATTTCTCCTGACCTGCTGCCAGAGGAAGCAAACTGTGAAG
ATGGCCTGTGAACTGAGACACCCTACCTCTGTTAATTCACCCTCAGGACT
GAAAACCTCTCTCCAATCACTTCTGTCGCTGTGAGACCCAGTGAGGTCCC
GGAGGCCCCTCCAGTTGGTCAGCCCTGGAGGGACTTGGCCGAGCTGCTCC
TGGTTTGGAGGGGGTCCCCCCCACCCACGCCCACGAGTCTCAAACAAGGC
GGCATTGGTGTCGTGGAGCGGGATGGTTCCGACGGCCCCCCTTCAGCGAT
GCTGGATCTGGGAACGCTTCTTGGGTTCGAGGGCACCCCCTTGTCCGCTG
TTCTGGTCCAATGCACTGGGAAGAAAGAAACGTCCAGGTCTTCTTGGAGC
AGAAAAGGGGCTGGGGTCTTCCTCTCCGGCCGCACAGACCAGCCCCAAAG
CCGACCCCAGAGCTGCCCACCCGGAGTCCCCTCACTGCTTTCCACCAAGT
TCAAAGTGAAGGGACCCCTCTACCTGTCTCTTCCTAAAGGCTTCTTTTTT
TGTTGTTAAATAGGAATCACACAAAGCTCTCGTGTCTGAATGGCCATTTT
ACGATTTTAAAATTGTTTTAACATAAAAAATATCTTTTTTCTCTTCTCCA
AAAATACTCCGGGCTCTTTCTCTTATAAGTCACTTTTTTTTTTTCTTCTT
CCCCCTCCTTGTAAAAAGTCCCCCTTGCCCTGCCTTCTTACATAAAGCAC
TTATTATGCGCCGAAAGTGCCCTTGAGACTGGAAGATGGGAGGCAACTAT
GAGGGGGGGAGGGGKAGGGGGGAGGGGGGAGGGGAAGATGCAGGCCAGAA
AGCACCGCATCTGAGAGGGGCTGTCTCTCTGGTGGGTGAAACAACAGCAA
ATAAGCAAGACACCCAGTAACAGTCTCCACCTGGGACCCAAGGCCCGCCC
CGCCTCCACCACCCTTCCACCCCAACCAAGACGACAGGATTCCAGCCCAT
AGAAAAACCACGGGGACAAAGGTTCTAGAAAATAGAAACTGTTGGGATTA
CAAAGGTACCCATTTCATCCATACAAACTGGTCTTTCGAACATCCTTGTG
AGAGTTTAACTGTAGTGTCCAAATGTTTAGGGGAAAAAAAAAAACAAAAA
AAAACAACCCATTGACGGGAGGAGTTTTTCCTCCCCTTTTGGTTTATCAC
AGCATTTTTTTTTCTCTTTTCATTTTGGCACAGCCTTTCCTGTTTTTTCG
TTCGTCTCAACCATCGGAGCCTGTTCTGGGTGGCAATTGATCCATGCACT
GAGTCAAGCTGGTGGCTTCTCGCTCCCCTGGGGCTGGACGTTTCAGGTGG
AAACCATTCCTTTTCCCCGCCTTTGACAGCACTTGCGTGTGTGTTTTCTG
TTTTAAACTTCTCGGTGGAGGTTATGAAAAAAAAGAAAAAAAGAAAAAAT
AGAGACTCCAGTGGCCAGGGATGACTTGGTCTCAAAGGACACTACCTAAG
GTCAGGTTTGCGCTGTTTCGGAGCTGACGGAATTGTGTCTGAATAGTGCT
ACAACCCCACACATCCACTGCTGGCCCGTCGGAATTCTCTTCTCATGTCT
ATAATATCTTTTCTGTGTGTGTTTACTTTCGCAGATCTAACACGCACACC
TGAAAGGAGAGAGAAAAGCGCCTGTTATCTTGAGAGGCAACATCAGATGC
CCTTAAGAGCACAGGATGGCACATGGCCTGTGGACCAAATCCGGCCCGCC
GTCCACTTGTTTATAAATAAAGTTTTATTCACACAGTCCAGAGGGTTCAA
TCAGACCAGCTCTAGCGGACAGGTGAGTGTCTAAACATCATTAGCACCCA
ACCCTAGGCCACAGCCGTCAATGGGTGTCACAGACCAGACCCCTACATGG
TGGAGCAGATGGCAACTGAAGCCTGAGCACACAGCTCGCGTAAGGGTCGG
GGGGCAGGCTGACAGCCAGCCAGCCAACGCCCTGCCCTACATCTCAGGGC
AATTCCAAGTGCAGAAAGCTAGACACCTCAGGGGACCAGCAGAGGGTTAC
TGGGACCCGGGAGACCTGTTGAATGGAAGGTTGGGCCTTCATCACCAGAG
GGTCTGGGTGGGGGCACAAAGGTTGAAGCCAAGGGTGGACCAAGTTAACT
GCTCAGCCTCATCACCTGTCATGTGGTGGTCACTTCTCCGCTGTTGGGGG
GAGGTCCTGAGCACCCTAAGACTTCCAGCAGCAGCCCCGGTTTCCACACC
AGGTGCCAGCAACCCCCCACCCACCCCCCCAAAAGCACTAACAACCCAAA
TGCCTCCAGACACCTCCAATGTCTCCAAGGGTGCAACCGATGCAGCTGAG
AACCACGAATTCCGCCAGGAAAAAAAACACACATCATTCAGGAAGCAAGA
AGAGACATTCACTCCCAGAGCACTGCCCAGCCCAGATGCTTCCTTCACCA
ACACACGTCTCTGAGGCCTGTCACAGCCCTGGGAGTACTCCTACGTGGGT
CTCAGGGGTCTGGACCGTTGTTGACCCCACCCTCCCCGAACCCTCCCTGC
TCCCCGCACCGCCCCCTCTCCTGCCTGGGGAAGCCCCAGGGGTGCTGCTT
ACGGAGCCGTCTGACGCACTGGCGCCCACTTTGTCGCCCCGGGCATTCCA
GCACACCTCGAAGATGCCCCCGGTGCCTCGGTAGCTGTGCACGAGACTTC
CGCTCTGCAAAGCAAGGGCGCCGTCACTCGAGAGGCGGAACGCTGGCGAG
GCCACCTGCCCACCGACCCCACCTGGGGATGTCAGCCCAGAAGCTTGCAA
AGCCACAGAAGCTAATAGAACTTTCAGTTAAAGGAGCTCTGGCCTGGCGG
GAACATGTGACTCAGATCTGTAGCCGCAGTGTGGGGGGCCTGCCTAAAGG
ACCCTTGGCTGCCCACCAAGAGAGACAGCCTCTATGGTTTCCGCCAGGCT
CACGCCTCCCGGGGGAAGGAGAAAGTCAGCAAGATTGGATACGTGCTTAT
ATTTCAACCAGGCTTCCGTCTCACTGAGTGTTCAAGGGAGCCTCAGAATA
ACTCAAAGAGTTTTATGGTTTAGTGGGGGAGAAAAAGGATGCAAAATGTG
GGAAGCTGCTGTATATATATACAGCACAGGGACCTCAGCTCGGTGCTCTG
CCATTACCTGGAGGCTTGAAGACGGGTGGGGAGAGGGAGATTCAAGAGGG
AGGGGACATATGTATACATATGGCTGATTGCCTTGCTGCATAGCAGAAAT
CAGCTGTAAAAAAAAGCAATTATCCTCCGATTTAAAAACAAACAAAAAAA
AGGGTGAGTAAAATGATTTGCTGAGCTGCTGATGGCAGGCAGAACCCAGC
TATTGATGGCAATTTTTTTGAAAGTCAAGCATCCTCCTGTGATGCGAGTT
CGATCCCTAGGTCAGGAAGGCGCCCCTGGAGAAGGGAATGGCTACCCTAA
CTCCACCATTCTTGCCTGGAGAATCCCGTGCACAGAGGAGCCTGGCGGGC
TACATAATATAAGTCCATGAGGTTGCAAGAGTCGGACAGGCCTGAGCGAC
CGAGCACACACATGGGCGGCTGTCAGCCTGGCTTCACATTAGGGCCACTG
TCGTGTCTCATTTGCCAAGAAGGCACGATTGCGTGAGACGGCCGCAGAGG
AGGAGCCCGAGGTCTCAGTGGACCACCCTCGGCCCCAGGCTGGCTAACGT
GGCATCCGTTACTACCTCCCTTGAGAAAGCATGTTCATCAGTGTCTGCCA
GGCCTCAGGGCACACCAACGGGGTGCCCGCTTCATCCATCCTGGGACCAC
ACGCAATGCCTGCTGCTGTCAATGGAAGGCAGTGGGTGCAAGGTTTCCTG
CCAAGGGCTCGGAAAAAAAATAACCCGAGTCCCTGGCAGGGGCGACCCAG
CATCCACAAAGAGCCTGGGGCAGATGGTGCTGGGTGGGCTTCCGACCGTC
CGGGGAACTGGGGATGTGTCTATGTCCGTGTCCGAGGCAAGGCATTCCTG
AGTGCAAACTCAGGTTCTGGGGTCACCAAGAAAGTGAAATCAAACTGTCA
GTCGCCTAGTTGTATCCGACTCTTGGCGACCCCACGGACTGTAGCCCGCC
AGGCTCCTCTGTCCCTGGGATTCTCCAGGCTAGAACACTGGAGTGGGCTG
CCATTCCCTTCTCCAGGAGAATCTTCCCAACCCAGGGATCAAACCCGGGT
CTCCCACAATGCAGGTGGATTCTTTACCATCTGAGCCACCAGGGAAGCCC
ACGGGTCAGTGAGAACTTGACTGTATTGGGTGTGGGGCTGGGGGGGTTGG
TGGGGACCCGGGCGGTGGGCATGCAGGGAGTTTACCTGAGTGTTCCAGAT
GTGCACACACTTGTCGAAGGAGCCGCTGGCCAAGTACTTCCCGTCGGGGC
TGAAGGCCACGCTGTACACGGGTTCCTGGTGCTTGGTCAGCGTGTGGAGG
CACACGCCCCGCTCCACGTCCCAGAGGCGGACGGTAGAATCGAACGAGGC
GCTGGAAAGACAGAGATGGTCACCGCGGACGCCTGTGGGTTCAAAAAAGG
CCACCACAGCTGTCGGCAGGTTCCCGCCGGTCTCCTGAGGACGTGACTCT
CACAGTGGGAGTGACTTCCCTTACATCTCAAAGACCGTAAGTGGGTATTT
CTTCCTCTTCTAGAATGTGACTCAACACGCACTGAACACAAAGCCACGGT
CCAGGGAGGGGACATGTATGCCTGTGGCCAATTCATACTCAGGTACGCCA
AAAGCCATCACAACATCATAAAGTTTTTCTCTTCCAATTAAGATAAATAA
ATTTTTTAAAAAGTCATGCAATAAATCTCTTTCTCTTTTTGGGTGCCATT
ATCAAGGGAACAAATTGACAGCCACACTCATTTATTTCTGCATTTCTGTG
AGACACAAATGCTACGTATTTGCTAAATTTTCTAAAACGGTCCCACCACT
GACAACACATGTATCGCTGCCAAGTGAAAAACAAAGCCAAAAATAAAAAG
CCGAGAGCCGTATTCGGGGAAATGGTTAAGTGAGCGAACACAAATTTTAA
TCCCACAGGAATTAAAATATAATCGCATCCAAATATACAATCACACTCCC
AGGAGCTGCAAATTTTAGTTATGTCTAAATCTGAAAGAAGAGCGTTTGAG
TGCATGCGTGCCAAGCTGTCCCATTCATGCCTGACTCTTTGTGACCCCAT
GGACTGCATCCCCGCCAGGCTCCTCTGTCCATGGGATTCTCCAGGCAAGA
ATACTGCAGTGGGTTGCCGTGCTCTCCTCCAGGGAATCTTCCCGACCCAG
GGATCAAACCCACGTCTCTTACATCTTCAGCATTGGCAGGCGGCTTCTTT
ACCACTAGGGCCACCTGGGAAGCCCACCCACCACAGAGCTGTTTCCCCAG
GCATTTCTTTCCTCTTCTGGGACTTACATCCATCCCTTTATGCTTGGCGC
CTAGAAGGCTCACTTACGGCCGTCATGGGTACCATGTGATTCAAAAGCAC
CTCACAAGTCATCAAACAAAACAAAACAAAAAAGATAACGTTCTGTTCCA
GACCTTTAAGTGCATTTCAGGCCAGTGGCTTGCAATAGGAGCATAACAGC
TCTCATGTTTTTCTGGATCAGCTTTTAGCTGATACAGGAATTCCTTTCTG
TGTTTTCTGAGAGACGGCCTTGGGTCAGGCACTTTCAGTAACAGATTCTT
CATCCCACTGCTATGTGACAAAGACAAGGGGAGGGGGTCTCCCCCTCATT
TACTGCAGGGCTGGGCCACCAGCCATTCTCCCCACTAACCCATTCTCTCC
AATTCTGCAGGAAAAAACCCACTGCCGCTTCCTCCCAAGTCCTGTCTTTC
CCAGGCGAGACCACACAACCATGCTTTGTGGGATCATGGTCTCCAGAACC
CTCGGTGTGTCCCATGTTCACTCCTGGAATCTCTTTAGCTTGTGTGCCTC
AAGGTTCTTATTAAAATATCCTACTGGGAGTGCAACACTGTATCCTGAGT
GAGGTCTGAGCAGGACTGGGAACACAGAAGTCTGCAGAACGCCGCCTCAG
TGAGGAGCTTTTGTGGACTTCTAACCGCTCTGCTCCCTTCTGGTGTGTCA
CTGACTGGCTAGTCCTCTGCATCCGATGCTGGTGTCTTAGGGGGTTTACT
GCACACACCAGGGGTCCCCATCCTCCGGGACCTGATGCCTGATGATCCGA
GGTGAAGCAGACGCGATCATAACAGAAATAAACGGCACAATAAATGTAGT
GCACTCGAATCATCCTGTAAACCTTGGAAAGTGATTTTCCATAAAACTGG
TCCCTGGTGCCAAAAAAGCCGGGGACAGCCAGGACGAAAGGTTAGGCTGG
ATCTGGTATTGGCCGGGGAACCTTTATCACCATCAACACTCTCACCACCT
GCAGCTCCGAGTTGGGCCAAGTAAACCAAGCCCGAAGCGAAGTGAAATGA
ACTGAAAGTCACTCAGTCGTGTCCAAGTCTTTGCGACCCCTTGGACTATA
CAGTCCATGGAATTCCCCAGGCCAGAATACTGGAGTGGGTAGCCTTTCCC
TTTTCCAGGGGATCTTCCCAACCCAGGGGTCGAAACCAGGTCTCCCACAT
TGCAGGCGGATTCTTTACCACCTGAGCCACCAGGGAAGCCCAAGAATACT
GGAGTGGATAGCCTCTCCCTTCTCCAGAGGATCTTCCTGACCCAGGGATC
GAACCAGGTCTCCTGCATTGCAGGTACATTCTTTACCAACTGAGCTTCCA
GGGAAGCCCAACCAAGCCTGGGGTTCAAGCTAAACTGCTTCCTTTCTTCA
GTTGTAGTAAAAACCTTCACCAAAGCAAAGTGCTGGGAGGGTGGATGTAA
GCAATTCCTCCCTCCAGCCTCAAGGTTAAAAGCATCCTTATCTCAACTTA
GCTATCATGTGATAACCTCTATCTCCTGGCCCTTTCACTTTGAGTTAAGT
TTCTAAGTAACTCTGAATGTGAATCACTCACTCGTGTCCAACTCTTTGCG
ACCCCACAGACTGTAGCCCGCCAGGCTCATCTGTCCATGGGATTCTCCAG
GTAGCTTTGTCCCATCTCCAGGGGAACTTCCCAACCTGAGTCTCCTGCAT
TACAGGCAGACTCTTTAGCATCTGAGGGCATGAACACGTTTGCAAGACAC
ACTGACAGCATCTCCTGGTCTCTTGGGGACACGAATGACTTTTACTGGGC
GCATGGCTGGCGCAGCCTCAGCCACCAAGGCTGCTTTCGGGAACCGAGAC
TGGGGTGCGGGCTGTCTGTCCTTTACCTCGCCAGCATGATGCTGGAGTTG
GGGTTGCTGGTGGCCGGCCCGGTGGGACTCCACTTGATGGTGTATATCTC
TTTGCTGTGCGCCTGAAGGTCGTGGACACACGTGTCCTGCTTCATACTCC
AGATCTACAGGACGCAACGGGAACACGGTGAGCAAAGGCCGATTCTTCCA
CTGCGGGGAGCTTCGGCACCGTTCCTGCCTGGAGCACTGCAGAGGTTTCT
CTGTCCCTGGGAAGCTGACGTCCAGCGGGGTGGGCGGCTGACAAGGGGCA
GGTGCTTCACAGGCAGAGGCGTCCGCTGACCACCAGGGGGCGCCCGTGAG
AAAGGCCAGAGAGTTCCATAGCACCCTGGTCTGATCTCGGAAGCTAGGCA
GGGTCGGGTCGGGTCAGGTCTAGTTAGTAATTGGCTGGGAGAAAGGCCTC
GGGAACGGAAGGAGGCTCCACGGGACCTCGACTGATAAACAGGGCTTCAC
TGCCGGGGCAAGCGCCACGTCGTTTGCAAAGAGAAAAAAACGACAGAGGC
AGCAAGAGTGCTCACAGCACAACGGCCAGCTGCAGGAGGAGCGCTGGGAC
CGAAGACACAGCACGAGGCCCCAGGGGTCACCAAGGGTCACACGGCCCAG
CCTTGCAGCCTTACCTTCAATGTCATGTCATCAGAGCAGGAGGCTAGCAA
CATGCCAGACGGATCCCATTTGATAGCGTTGACCTCATTCTGCAAGAGAG
CAAAAGGTTTAAAAAAAAAAAAAAAGAAAGAAATGAAGGCCTGGGTGTAG
TAAACACAAGAGTCCAGCAGAGGGCAGGAGCGCTCTGTCTTCTCTGCAAA
AAGCCTGGGAGTAGTAAACACAAGCGTCCAGCAGGGGGCAGGAGCGCTCT
GTCCTCGCTGAAAAAAGCCTGGGAGTAGTAGGCACAAGCTTCCAGCAGGG
GGCAGGAGCGCTCTATCCTCACTGCAAAAAGCCAGGAACCTGTAAAACAA
GTTGAATGCAGAATTTCAAAGAATAGCAAGGAGAGAGAAGAAAACCTTCC
TCAGCAATCAGTGCAAAGAAATAGAGGAAAACAATAGAAAGGGAAAGACT
AGAGATCTCTTCAAGAAAATTAGAGATACCAAGGGAACATTTCATGCAAA
GATGGGTATGATAAAGGGCAGAAATGGTATGGTCCTAACAGGAGCAGAAG
ATATTAAGAAGAGGTAGTAAGAATACACAGAAGAACTATATAAGAAAGAT
CTTCATCACCCAGATAATCATGATGGTGTGATCACCAACCTAGAGCCAGA
CATTCTGGAATGCTTTAGGAAGCATCACTATGAACAAAGCTAGTGGAGGC
GATGGAATTCCAGTTGAGCTATTTCAGAATCTAAAAGATGATGCTGTGAA
AGTGCTGCACTCAATATGCCAGCAAATCTGGAAATCTCAGCAGTGGCCAC
ACGACTGGAAAAGGTCAGTTTTCATTCCAATCCTAAAGAAAGGTAGTGCC
AAAGAATGCTCAAACTACTGCACATCTGCACTCATCTCACACGCTAATAA
AGTAATGCTCAAAACTCTCCAAGCCAGGCTTCAACAGTACGTGAACCGTG
AACTTCCAGATGTTCAAGCTGGATTTAGAAAAGGCAGAGGAACCAGAGAT
CAAATGGCCAACATCTGTTGGATCATCAAAAAATCAAGAGAGTTCCAGAA
AAATATCTACTTCTTACTTTATTGACTATGCCAAAGCTTTTGACTGTGTG
GACCACAACAAACTCTGGAAAATTCTTAGAGATGGGAATACCAGACCACC
TGAACTACCTCTGGAGAAATCTGTATGCAGGTCAGGAAGCAACAGTTAGA
ACTGGACATGGAATAACAGATTTGTAATAAACAGGGAAAGAGTACGTCAA
GGCTGATATTGTCACCCTGCTTATTTAACTTATATGCAGAGAACATCATG
AGGAACTCCGGGCTGGAAGAAGCACAAGCTGGAATCAAGATGGGCAGGAG
AAATATCAGTAACCTCAGATATGCAGATGACACCATCCTTATGGCACAAA
GTGAAGAAGAACTAAAGAGCCTTTTGATGAAAATGAAAGTGGAGAGTGAA
AAAGTTGGCTTCAACATTCAGAAAACTAAGATCATGGCATTCGGTCCCAT
CACTTCATGGCAAATTAGATGGGGAGGGAAACAGTGGCAGACTCTATTTT
TTGGGGCTCCAAAATCACTGCAGATGGTGACTGCAGCCATGAAATTAAAA
GACGCTTACTCCTTGGAAGGAAAGTTATGACCAACCTAGACAGCATATTA
AAAAGTGCAGACATTACTTTGCCAACAAAGGTCTGTCTAGTCAAAGCTAT
GGTTCTTCCAGTAGTCATGTATGGATGTGAGAGCTGGACTATAAAGAAAG
CTGAGCACCAAAGAACTGATGCTTTTGAACTGTGGTGTTGGAGAAGACTC
TTGAGAGTTCCTTGGACTGCAAGAAGATCCAACCAGTACACCCTAAGGGA
GATCAGTCCTGAATATTCATTGGAACGACTGATGCTGAAGCTGAAACTCC
AAAACTTTGGCCACCTGATGCAAAGAACTGACTCACTGGAGAAGACCCTG
ATGCTGGGAAAGATTGAAGGCGGGAAAAGGGGACGACAGAGGATGAGATG
GTTAGATGGCATCACTGGGTCAATGGACATGAGTTTGAGCAGCTCCAGGA
GTTGGTGATGGGCAGGGAGGCCTGGCGTGCTGCAGTCCATGGGGTTGCAA
AGAGTTGGACACAACTGACCAACGGAACTGAACTGAACTGAACTAACACA
AGCTTCCAGCAGGGGGAAGGAGCCCTCTGTCGTCTCTGCAAGAAGCCTGC
TTGCATTCACCCTGGATAACCAAGCAGCACCCCAGGTTTCAGGCATGTTT
CTCTTTCTCCTTCCTCCTTGATCCAAAGTGCACAGTCATACGCCCTACAC
AGCCAGCCACCCTCAGCTATATTTCTCCAAGGGGGGAAAAAAAAATCACA
TCTAAGAAGGAAGCCAAATGAATTTATCTGGAAACCAATGTAAAACCAGT
TTGTTATTTCAAAGCTGACTTTAAAAAAGTAATTTTCGTACTTTTTTTTC
CCCTTTATGTCTTCAGACATTTTTATAATACCTACTTTAGTGTCTTTCCT
GCAAAATCCAACACCTGGCTCATGCCAGATAATTAAAAAAGAAAAACAAA
ACAAAACAAAAACACCAGTGCCAACCCAGGTGGATGACTGACCACCGGAG
GCAGCTTCAACAGTCCCAAGGAGGCTAGAGGAGAATGAGTTGGCAGTCAC
AGTAAGTAGGCTTTTAATCCTAGGCAGAGACCAGCCGGTTTCCCAGCTCC
CCACACACCTTTCGTGAACCCGAGGCCCCACACTCAGGACTCACACTCAC
CGTGTGTCCCTGGAATGTTTTGACGGGGCGGTCACAGCCGAGTCTGCAGA
CGTGAATACACATGTCCGTGCTGCAGGAGGCGAAGGTAGTGTTGTTCTGC
CAGTCCACGTCGAGAGCAGGGGCTGCGGAGAAGCAAAGGGCGGAGGGGTG
GGGGAGAGCGGACAACACGGGCATTGAGCAGGTGTGCACAGAGCTCAGCT
GACTCAGAGGCGGCAGCAGCAGAGCTGAGAAACCTGACAATGTGATTAAA
GCAAGACTCGCGGTGCCCACACAGCCTCGGTGCATCACGGACCCTGAGGT
CTGCGGTTTACATTTGCAAGGATGACCAGACGCCTCCAGAAAGAGAGCCG
ACCCCCACCAAAGCGTCACCCAAAGTGATGCTTTCGGTGCCCTGAGCACA
CAGCAACTCGCTCCCCTGGTGCAGGTGAGATGGTAGGGGAGAAGCACCTT
GCTGCTCCCGGGAACGTCACCGGGCTGGGTGATAGATTTTCTTTCGGTGG
GGAAGGGAGAGAGCGGAGGAAAAAGACTTCACAGCCTATGTTCAACATGT
GTGTGTGTCTCTCGCTCAGTTATACCTGACTCTTTGCGACCCCGTGGACC
ACAGCCTGCCAGGCTTCTCTGTCCATGGGATTCTCCAGGCACAAATCCTG
GAGTGGGTTGCTATTCCCTTCTCCAGGGAATCTTCCCTGATCCAGGGATG
GAACCCAGGTCTTCCGCACTGCAGGCAGATTCTTTACCATCTGAGTCACC
AGCGAAGCCCCATGTTCAAGGTATCAGGGTTGAAAGAAATGCCCCATGAC
CCGTTACTGCATTCTACAACTTGAAGATGCTGAGTTGACGAGAACATCTG
AGGGGAGACATGTGGCCTTAGGATGGACACCGTCTCTACGTCCTGGGCCT
TTCTCAGAGGCCGCTGGCTTGAAGACCTCCGTGAGGGTTTGGGGGACATG
ACGGCTTGCTTCCCTTCTGCCATCGTCCCCAGGAGATGACTCAGCACCCT
CATTCTCTCCGGACACGTCCCTGTTTCTACAGAAAAGGAGGGTTAAGTCC
TGCAAGCCTCTGCACAGAACTTTGCCAACTGATCAATACCTAACCTTGTC
TCACGTGTTTCTTACACGTAAGTCAGCCCTGAAGTGGGACGAGAATCCAG
CTCACCCAGGACATGATAAATCCGACAACCCCCTTCTAACCTTAGCTTCA
ACATATATTTATTAATTTTTTAACATGTATATTTTGGCCAGCTGATGCAA
AGAGCCGACTCATTGGAAAAGACCCTGATGTTGGGAAAGATTGAAGGCAG
GAGGAGAAGGGGACGACAGAGGATGACATGGCTGGATGGCATCACCGACT
CAATGCACATGAGTTTGAGCAAACTGTGGGAGTCGGTGATGGACAGGGAG
GCCTGGCGTGCTGCAGTCCATGGGGTCACAAAGAGTCAGACACGACTGAG
CCACTGAGCAATTTCTGGCTGTGCTGGGTCTTGGCTGCTATGCGGGTGAC
TGTCTAGCTGAGGTGTGCGGGCTTCTCACTGAGCCAGCTTCTCTTATCGG
GGAGCACAAAGTCAGGAGTCGTAGGGCACGTGTTTACTTGCTCCGTGGCA
CACGCTATCTTCCTGGGTCGGGGATCGAACCTGAGTCCCCTGCCTTGCAA
AGGTAGAGTGTTAACCACTGGACCACCAGGGAAGCCCCTGTTCTTAACCT
TCAAAGCAGAGGTGCACCCCGTCCACCCTCCTGCCTTCCAGAAGTGCAGG
ACAGATCCCATCAGCTTCTAAATCTTAGCACCACCCCTCACAGAGCCTGC
AGGGGGAGGGTTTCACGGAGAGGCAACACTGGCTTTCTCAAGAACCCAAA
AAGGGGGTTCAGCACACGTCCACGGCTCCTGCTGAATTTGCCACAGGCGC
CCAGTGGAATGAAGGAGCCGGATCCACTTGGTCAGCAGGACGATCCTGGG
AGTGTGGAAGCAGAGCCCGCAGCTGCTCTTGTTTATAACACCGCATGCAT
GAGCCAAGTCACAGATTTCCCGGGCCGGCCTCTGCCTTCAGGCAAGCAAT
TAATACGCCTGGAAGGGTGACAGTCTGGCACGCGGTTCCGGGGATGGTTA
GCGGCACCTGGCCTTAAAAGCGAATGTGTGCTGAGCCCCGGCCATCTAAA
CAGAGGACGCTGTGTACTCAGCTGACCGGGGTCGTCGGTCTGTGTGCACT
GTGTGGCTGGGACCCCGCCCTCCCCCGACCAGTCACGAGGGCTCAGCGGT
TCTAGGGGGTGTGGGTGACCTGCTCACACTTGGCCAATGACGTTTGAGCA
TCAGCACCGCGCTAGGCACAGTCGTGCTTCACGTTGCTTTTTTCCTTTTA
ACTTTTACTTGAGAAAAGGGTATAGAATTGATGCTTTGGAACTGCGGTGC
GAGAGTCCTCTGGACAGCCAGGAGGTCCAACCAGTCCATCCTAAAGGAGA
TCAACCCGGAATATTTACTGGAAGCAAGGATGCTGAAGCTGCAATATTTC
TGGTCACCTGATGGGAAGGACTGACTCATTGGAAAAGACTCTGATGCTGG
GAAAGATTGAAGGCAGGGGGAGAAGGGGACGACAGATGGTTGGATGGCAT
CACCGACTCGATGGACATGGGTTTGAGCAAACTCCAGGAGATGGTGAAGG
ACAGGGAAGCCTGGTGTCCTGCAGTCCATGAGGCGGCAAAGAGTTGAACG
CAAGTGAGCAACTGAACAGTAACGACAAACAGAATCCCAGGAAGGTGCAG
AGATGTTACAGGAAGGCTCTACCTTGTATCCGTCACCCACTGGTTGCACG
TCGCAGCCCTGCAGCACTGGCTGGACTTGGAAGGTGAGTGGGTCCAGACC
CAGCTGTGTATGCAGTGTCACAGGGTTTGATCTCAAGGGCCGATCCATGT
AACCAGCACGGTGGTCAAGAGCCGCAAGCGTGTTCCACATCACCAGGACC
TCCCTTGGGCCATGTTAGTTGGCCCACCATGCTCTCCCTGGACTGAGATG
TAAATGCTATCTCACCCAGGATGTGTTCCTTGGAGTCTGACTTTATTTTT
TTTTCAAACTGCAGAAGCCCTCTGAGATCTATCCAAGCCTTTGGGTATTA
CCCACCTCATGAGTCACTCAGTTGTATCTGACTCTTTGCGACCCCATGGA
CTGTAATAGCCCGCCAGGCTCCCCGTCCATGGGATTCTCCAGGCAGAACA
CTGGAGTGGGTTGCCATTTCCTTCTCCAGGGGAATCTTCCCGACCCAGAG
ATCAAACCTGGGTCTCTTAAATTGCGCGCAGATTCTTTACCATCTGAGCC
ACTAGTTAGGTCCTGTCTGAGCCACGATTTGACCCTCATGGCTCGGGCAG
TATTATCAACAGTCTATCAATAAAAATGAGTGGGTTGCCATGCCCTCCTC
CAGGGGATCTTCCCGACCCAGGGATTGAACCTGGGTCTCATGTATCAGAA
ACGGATTATTTTCTGTCTGAGCCACGAGGGAACGTCCTGCACTTGTTTTT
AACCTGTTCTTTCTCAGTGCTGAGCGGTGGAACCCTGTTAAGCTCTGAGG
CCTAAGGTTTCTTGCTGACCGAGGGCACCCCCACCAAGCCTTCATGGGGA
ACAGCTCTGGAGAGGCAAGTCAACTGCTGATTATCAACGCATCACACACT
GAGGGATGGTTGTCCCTACAGCTTAAAGAAAAGCACGCTCCCTTGTCTTC
CATCTTGGACAGTGTCTACCACACCACTTCACGGTATAGATAAAGAGCAA
ATAGCACCCAGAGCCAACCCAGCAATGGGATACGGGGCGACCCGTGGGCA
GCAACACCCCAGGTGAGAAGCTCCCTGACGGCAGAAGAGACACGTGGTCC
GTGGGATAGAAAGCCTGGGACCTTGGGAATGCTCTGGGGAAAATTTACAT
CCTGCTGAAAGACTCAATACAAGAGTCCTCAGGGTGAAAACTGGTACAGG
AAACTTATGGGCATATTCCCCTCTGAAAAAATCAGAAGCCAGAGCTGTCT
GTGTTACAGACATCCCCCCATCACGTGAGCACACTGGTGCTCAGCACGAA
GAATCAGAACTCTGGCAGGGTTCTGCCCCGTCGCTGAGTCCCTTCCCTAC
AACAGAGCTTGAGATGCTCTTAGCCTCTGTAATGAGGCTGCGATCCCCGA
AGGGGGCTCACGCCAAACCCGACAGAAATCCAGCAGGCTCACCTGCCACG
GTCTGCTCTGTCCATGGCACAGACGGCCGGCTCGGAGGAGGTGACACGCA
TGGATCAAATCATGGAGACCGGGTTGTCAGGGGCTGGGAGAGGGCACTAG
AGAGCACTTTCTTAACAAGTAAGGGGTATCCTTCTGGAGTGACAGGAATG
TTCTGGAGCTAGACAGCGGTGAATGCTCTGCACATTCTGCCCACTGGAAA
CACACACATGGACACAGACACACAAACCTACACACAGAGGCACGCACAGA
GACACACGGACCACCCCCCCCACAAGAGACACAGACCCCACAAACAGAGA
CACACACCTACAAACACACACGCACCCACACAGACACGCCCCTTCAGAGA
GATACCCACACCCACAGACCCCCCAAAGATACATCCCTTCCCACACACAG
AAACACATCCACACACAAACCCCTTCCACAAAGAGAGACACACACCCACA
CCCACACACACACACACAGACCCCTTCCACAGAGAAAGACACACCCACAC
ACAGACACACACCCACACACAGACCGCTTCCACAAAGAGACACACACCCA
AACACAGACACACACCCACACACAGACCCCTTCCACAAAGAGACACACAC
CCACACAGAGACGCCTCCTTCACACACAAAGACTCCCCTACAGAGAGACA
CATACACAGACCCCCCCTGCAACACATACACATTGCCACACAGAAAGACA
TACACAGAAACACACAAACAGACCCACATACACACACTCACAGGTTGGGC
GCCCAAGCTCCCTGCTCTTGGAGCCCTGCCCACTTCCTGGTGGGGAAACA
CAATTCTACAAAAGGCTCAGCGAGAACACAATTCTACAAAAGGCTCAGCG
ACTCCCACCTTCATCACTGCTGTTAAGAGTAAAACAGTATGAAACACCCC
CAGCCAGCACCCCCAGAGACGTGTGCAGCATGGAGTCCCCGAGCAGAGCC
ACGGAAGACATGGGCTGCCTGCCTCTCCAGCAGGGCGAGGTATGCACGGG
GCCCCTCCCGGGGCCTGCTGGTCGAACCAGGAGGGAACGAAGGACTGCGT
GCCTTTGTCACAGTCATGTTGAGGGCCCTGCGGGGAACCTCTCCTCCCAG
GCTGCTTCTGCAGGACCACACCCCTCCCCTCCTGCTTGGAGCCCTTCCCA
GGCAGCGAGGTGGGTCGCGCTAGCTTGGGGAGTCCTGGGGGCCAGCCCTG
TGGTTCTGAGCATCACCAGGAACCTGGATCTGCCCAGTAGGAGAGAACCT
CGGATGGCGACCAACACATCACCTCAGGGCCACTCAATGCATCACCCAAC
GTCCACCTTGCATGCTCAGCACTGCTCATGAGGCATGCCTTCTTCCGAGC
ATTCACCGTTACGTTAAGAGACCGTTCTCAGGACTCCGAATTCTTATTTT
TGAATTAAAAACAAGGAAAGTTAAAAAAAAAAAAAAGTATTAAAAAAGTA
CTCACCGGAATGAAATGGAAACTGTTGTTTGGCTTCTCCTGTGTGAGCAT
CCCAGATTATTGTTGTCTGTGCTCCGCAAAAAAAAAAAAAAGAAAAAGAA
AAAGAAAAAAATTTAGTTACTGGTTCCTCCCGGAAAGTTATCTCCAGAAA
AATTTGTCACACATGAGAACACAGCGAGCCCTTCTTTTTTTCTAAACTAG
CACTGCTCAACTTAAAATTTTCTAGTAACCACAGTAAAAATTAAAAAAAA
AAAAAACAAAAAACCAAAACAGTAAAACTTATTTTCACAATATACAGTTT
ATTTCACTCCAGCACAGTGAAAATGTCACTTCAACGTCAATCAATATATT
CGCAGCATGTGGCCTGTGTCTTTTATGCCCACTCTTTGGAATCCAGGGTG
TGTCTTACACACTTAAATCACCCAGACATCACACTTATGAACTCAGCCAC
GCCTGTCTGATTATCATACTCCTCCGGACGACAGCAGCCGTAACTGATAG
GCAGGAAGCTACCAATTTTGGTGGGAGTCCCATCTTTATCCTCAAACTTG
AATATCTATCATTAAAAAAAAAACTTTTCTTTTTAAATGAAGAGATTTTT
GGTTGCACAGGACGTGAGATCCCAGCCATGCATCGAACATGTGTCCCTCT
GCACTGGAAAGTGTAGAGTCTTAACCACTGGACCCAGCAGGGAAGTCCCA
AACTTGAAAAATTATTAAAAAACAAACAAACAAACAAAGCATATCTAAAA
CTCCAGTTTGGACCAATTTTCCAACTGAAACACTGCAGAAATGGTTATTT
CTGAAAATCCCCCTGACCGTCACTCTGTGAAGTGGGGACACTACACAGGC
TAGAGGAACTGGGTTCCACTGGGGTCTGGCCCTGATGTATTGCTGTACAT
ATTAAGAAGAAGAAAAAAAAAAAAAGATCTCCACCCCAGAAGACAGAGGA
AGTCACGAATGCTTCTGAACACATTATAGTGTTGGGGAGACACTTGGGGA
AGAAGCCGGAGGCAGTTCAATGCAAAACATTTGGAATTAATTCTCAACTG
GAATTTTCAGCTAAATTGGAAACTTGACGCCTCCCTGTCATTGACATTTT
AACAAAGATCTTCCTTTAGGAAAAACTGGCTCTACTTTGTATCAACAGCG
CTAATGTTCCTGGCGAGATACTGACAGCACTGAGTGTGTTTGAAAAACTT
CAGGCCAGCTCCAGCGCTAAGTCGCTTCAGTCGTGTCTGACTCTCTGTGA
CCCCAGGGACTGTAAGTCCATCAGGCTCCTCTGTCCGTGGGATTCTCCAG
GCAAGAACACTGGAGTGGGTTGCCATGCCTTCCTCCAGGGGATTCTCCCC
ATCCAGGGATGGAACCTGCGTCTCTTATTTATAAGTCTCGAGCATTGGCA
GGCGGGTTCTTTACCACCAGCTCCACCTGGGAAGCTCTGGACCAATTAAC
ATTTTTATCCCCCGCTCCACCGTGTGGTTTATAGCATTTTAGTTCCCTGA
CCGGGTACTGAACCTGGGCCCCGAGCAGGGAAAGCGCCAAGTCCTAACCA
CTGGACTGCCAGGGAAGTCCCAGAGATTTTTTTTTTGATGGAGATTGGTC
CTGAGTGTTCATTGGAAGGACTGATGTTGAGGCTGAGACTCCAATCCTTT
GGCCCCCCGATGCGAAGAGCTGACTCATTTGAAAAGACCCTGATGCTGGG
AAAGATTGAAGGCAGGAGGAGAAGGGGACGACCAAGGATGAGGTGGTTGG
ATGGCATCACCAACTCGATGGACATGAGTCTGAATAAACTCCGGGAGTTG
GTGATGGACAGGGAGGCCTGGCGTGCTGTAGTCCATGAGGTCGCAAAGAG
TCGGACACTACTGAGCAACTGAACTGAACTCTTTTTTCTTAACTGAGCCA
GTCACAAGTCTGGCATGGCAGAGAATATATAAACACTTTCACAATTCTGA
CACAACTTCACATACCACATCAGTTGTTGTAACAACTTACTATCAGTTCA
GTTCAGTCACTCAGTCGTGCCTGACTCATTTGCTATCCCAAGGACCTCAC
AACTTACTATGTGTTACCTTAAACACACACTGGACAAAAGTAAACATGAC
TTATCCAAAAGCTTTTATGGATCAGAAATACAGGTTAGCCACTCTGGGTT
AAGCACGCACCGAGGGCCAAAGCCAGTATCCCTAACTTAAATTTCATCCC
TGAGAAGATATCATTTTCAAGAACACTGTTATTATTCCTGCCACAGTTGA
TTCATCATGGTAGACAGTCTTTCACTCAGAGGTTCTAGAATTCCAGAAGA
TCAAAACCACAGAAGCTCACAAAACCTCCAAACAAGGCCCCTTTGATTTC
TTAAGGGGGAGGTAGAATTTCCAGAATTCAAAGAAACCGTGATGTTTCCC
GAGAGATTAAACATAGCTGAAAAAAGCAAGGTATCTCACGCAAACACTCA
CTTTGTCTACTCCAGCACTCAAAATGTAATTGCCCTTCTTGTTCCACTTC
AAGGCGAAGATGGGGCCTTTATGTTGGCCTAAGGTGCTGGCCAGGTTACC
TGGTAGGTTAAAAACAAAGAGAGAAATCAGGGTGACTTCTGAGACATGCT
CGGTCTGTACCCCAGATGATGGCAACTGAAAGTACCCCCTCTTCCAACCC
TGACGCGAGGGAGGCGGGGCCAGGAGGACTTACCATCCTCTGTCCATATT
CTTGCAAAACCATCATAGGAACCCGTAGCCAGCAGTGTCCCGTCACTCTG
TGGGCCAGAAACACACACCAACACAAACTATTTATTACAGCCTGCAGGCA
GGGCCTCCATTCCCCGAGCAGGGGTCGGTCAAACTCACGGCCCCTGCAAC
AGAGCTGCAGACTCCTGACCCCTGGGCCACCAGGGGAAGTCCCAGGCTTC
CTTCCCCCAAAATCATCACAAGGCCAGGGGGCGAGGGAAACACACCGCAC
CCCGTCACCAATGACGACACCAATAAAAGACACCGTACGAAAAGTTACTG
AGTGACTTTGGGAGACCTTGACGAGCTTTGAAATAGCTGCAGATACTCTG
ACAAGAGAAGAGTTTGTGATGAAATGACAGGACATCCAGGAACGTGATAA
ACCGTTGGTGGGAATGCAAACTAGCACAGCCACTACGGAGAACAGTGTGG
AGATTCCTTAAAAAACTGGAACTAGAACTGCCATACGACCCAGCAATCCC
ACCGCTGGGCATACACACCGAGGAAACCAGAACTGAAAGAGACATGTGTA
CCCCAATGTTCATCGCAGCACTGTTTATAATAAATAGCCAGGACATGGAA
GCAACCTAGATGTCCATCAGCAGATGAATGGATAAGAAAGAGTGGAACAT
ATACACACTGGAATATTACTCAGCCATTAAAAAGAATGCATTTGAATCGG
TTCTAATGAGGTGGATGAAACTGGGGCCTATTATACAGAGTGAAGCAAGT
CAGAAAGAAAAACACCAATACAGTATACTAATGCATATATGTGGAATTTA
GAAAGATGGTAATGATAACCATGTATGCGAGATAGCAAAAGAGACACAGA
TGTACAGAATAGTCTTCTGGACTCTGTGGGAGAAGGCGAGGGTGGGATGA
TCTGAGAGAATAGCATTGAAACATGTATATTATCATATGTGAAACAGATC
GCCAGTCCAGGTTCAATGCATGAGGCAGGGTGCTCGGGGCTGGTGCACTG
GGATGACCCAGAGGGATGGGATGGGGAGGGAGGTGGGTTCAGGATGGGGA
ACATGTGTATACCTGTGGCAGATTCATGTCAAGTATGGCAAAAACCACTA
TAATATTGTAAAGTAATTAGTCCCCAATTAAAATAAATTAATAAAATAAT
ATCAAAAACAGAAAAAAATAAAAAATAAACTGTACAGTCCAAAAAGAAAA
AAAGAACGTGATAAAGAGAAAGTGAAAAGATGCTGGAGAGCTGTGTGTGT
GTCCGGGCACAGATGCTGAGGGTCGGGTGGGAAGTTCGCATCTCCCTGGG
CACACCTCAGGGTACCCCCACATAACAGGCTTCCTGATAAAGCCGCCAGA
CTCTTCCTGCCGCCGACGTGCAGCTTACTGAACACCATCAGGCTGGCGGT
CGGCCCGGACCCCCGGGTGGGCTGACCCACAATCCCGTCCCCTTCCTGGG
GTCCGCCCCAGCTCCGCCCAACACCACAAGCTGTCACCGAGGTCGTGGGG
ACTGCACAGGTGTCCCCGACGGCGTCCGGGAGGGAGGCAGCTTACGTTCC
AGTCCAGCGAGGTGACGTCTTTGTTACTGGGGACGTCGTGGCCCCCCTCC
CGGATACAGTGCCTCAGCACCAGCTGCGTGGAGCCCCCGTTGCTGTTTTC
ATTCAGGTTCCATATCCTGGCAGTCGAGTCTCCGGACCTGCGCAAGGAAC
ACAGCTGGCCCTCAAGCTTCCGGGCCCAGAGCAAGCAACACCCACTGTTG
CTTGTCGCCTGGCTGGGTCTCTATCTTTTTAATGTCGCTCCAAAAAAAAA
ATCACCGTGGTTTTCTCTCTCCACTCTTAGGTGCCCTCATCATTTTAAAT
GTCTGTGCATAGCTTTCCACACAGCTGAGAAAACGGCTGAATACGGACTT
TTGCACAGGCTTAGACACCCCAAGGGTTCCCCCCCGCTCAAAGCCCTGCT
CTGTACGCTCTGAGAAGATTTCACACATGACGTAGAAAGGGAGTGTCTAT
CCCAGGAGGAAGGACTCGGACACCTGGACCCCGGGCACCCCCCAATCTGA
TCCTGGAGGACTCGGACCCCTGGAACGTGGGCACCCTCCATGTGACCCTG
GAGGACTCGGACACTTGGACCCTGGGCATCCCCCAACCTGACCTTGGAGG
ACTTGGACACCCACACCCCAGGCACCCCCCAACCTGACCCTGGAGGACTC
GGATACCTGGACCCTGGGCAACCCCCAACCTGACCGTGGATGACTTGGAC
ACTTGGACCCCAGGAAGCCCCCAACCTGATCCTGGAGGACTCGGATATCT
GGACCCCGGGCACCCCCGGCCTGACCCCTTACCCCGAAGCCAAGAGGTCA
CTGACAGGGTTCCAGGCACAAATGAACACCTCGGACTCGTGGCCCCGAAG
GACGGTGGCTTTGCTGGGCGGGATCTCCACGTCTCCATCTATTTCCATTG
GTTTCGAGTGATTATCTGTCGCGGGGAACAGGACAGGACACGTGTCTCAC
TGACATGGAAGAAACCCATCCATCCTCAGAGCATTTAAGCTTGACAGATA
ACTTCCTCCGCTGCACTGCCAGGTCACTTGTGACAATTGGAACCAGGTGA
TGGCTTCTGTGAAGTCTTCACAGTCTCCAAATTTGATAGGGGAATTAAAA
ACTGGTGTTTATTCTTTGTAACTAGGGCCAGGCTCATAGGTAATAACAAT
GGGAATGAGACTGGGCACAAACAGACAGCTGTCTCTTGAGCAGAATTTGA
AGCTTTCTTCTCTGTCTTAGTGAACTCCGGGAGTTGGTGATGGACAGGGA
GGCCTGGCGTGCTGCGATTCACGGGGTCGCAAAGAGTCGGACACGACTGA
GTGACTGATCTGATCTGATCTCTGTCTTTTATGTCTACAGTAAAAAGACG
CATATTTTGTTACATTTTCCTAAGAAGTCAATGAGGAGAGGGGGACGCAG
AGGATGAGACGGTTGGATGGCATCACTGACTCAATGGACATGAGCCTGAG
CCAACTCCGGGAGATGGTGATGGACAGGGAAGACCAGCGTGCTACAGCCC
ATGGGGTCTCAAAGAGTCAGACACCACCGAATGAACAAGAGCAACACAAG
TGAAATTAGGAGTTTCTGAATCCCAAAGGCCGTCTCACTCTTTGTTATAT
GCCTAGAACTTAAGTTGTGCTGTTGCTGTTTAAGTCGTTGTGTCACGTCC
GTGTCTTTGTGACCTGGCTTCTCTGTGCACAGGGATTCTCCAGGCAAAAA
TACTGAAGTGGGTTGCTGTTTCCTCCTCCAGGGGATCTTCCCGACCCAGG
GATCGAACCCGCATCTCTTAGTATCTCCTGCATTGGCAGATGAGATCTTT
ACTGGTGAACAACTTAGCAACTGAACAACAAGTCAGTTGCAAATACTGCT
GCTAGAATGCAAGTTACAATTCAAATGACAGTGAGAATAATTAGGATATA
AAGTTTCCTCAGCAAAAGTTCCTTCTGTTGACGGGACAAGTGATTTTATA
AGACATCTTCCTGAGGCGTCACCTGCTTACCTACGAAGCTCCCACACGGA
CACGCAATCCTTCTTCCAGAAGGCTCACCACTCACAACCCCTTGGCCTGC
TAACAACCTCATGGTGTTGGGACTCCAATGCTATTTTCTTACTGGAGCTA
AGTGTGTTCCAGGCTTTTGAAGCCATGCAGACACAGCCCAGATGACCCAA
TGGGGACTCCAGGTTGTGAAAGGATGGTGGCAACAAGTCATGGTAGATGC
CGCACAGAAGAGACTCATCCTGGATCATGCTTTAGCGTTAAGTCGCTCAG
TCATGCTGACTCCTTGCGACCCCATGGACTGTAGCCCGCCAGGCTCCTCT
GTCCATGGGATTTCCCTAGGCAGGAATACTGGAGTGGGCTGCCATTTCCT
CCTCCAGGGGATCTTCCTGACCCAGGGATGGAACCTGGGTCTCCTGCCCT
GTGGGCAGATTCTTTACCATGTGAGCCCCCAGCAAAGTCCTGAAAAGTGG
ATCCACTTTTTCCCCTTCTATGTGGCACAAGAAAGAAGGTTCTTGAAGGT
CAAAATGAAGTCTAACTTCAGACGCTGTAAGTCTCGCTGAAGCCGCCTTT
TAATAAGAGCAGGTGGGACAGAGTAGAAGTTGGAAAACAGTGGACATTGC
ATGCATGTGTATGTGTGTGTTGCGGGGGGAGGCTGGTGGCCAGCCCTCAG
GTGGAAAGTAACCCCATTGCAGAGGCCCCCTCAAACGAAGGAGGAGCTGG
GAGCTTCCAGCGCAGCTTGAGGTATGCACCTGTCTCCACGTGGCTACGCT
CCCTTGGCGTTTTAGATTGAAAGACATGGAGTTTAAAAAGTTTGCAAGAT
TTCTGTAACAGCAGTAGGTACATAGATTTCTCAAGCCTCACCAATTACGC
TTCCTTTCTGGGGTGGAAGGTGAAGAAAACGGGGACGTTCACCTTCTGGG
CTGTAGCTGAAGCAAACGAGGATGTTCACCTTCTTCCTGTTTTACTTTTA
GCACCTTGACGGAGAGACCCCATGTGACTGGGCACACCCACGAATGACGT
GCGGGCACAAGCCACGTTATCTTTTCTTCTCCTTTCCTGGGACCCTGTCC
TTGCGAACCTTGTGAACAATGATTATGGGGCCTGCTGATGGGAGGTCATT
TGATTCGACACTGCACAGACAGTCTTGAAGCCATCCTGCATGGCAGCCTG
GCCCTTCTCTCTGCCCAGTGGTAGAGCCATGTCCCAACTCCAGTGGGGAG
AGGCCTCGGCCCGGAGATGACAATCTTCCCCCTCGGGTGTGCACAGCACT
TCAGAGGGAAGGGGCRTTCAAAATAGGGAAGAAGACACACTCACTGGCAG
AGACTTACATGCATGTACACACACACTCTGAACGTAGAACTTCCTTGAAG
CTGTCTCTGAATTTGTAAGCAGGAGGTTCTCTGGCCTTCCCTGGCGGCTC
GGATGGTAAAGAATCCACCTACCAATGCAGGAGACGCAGGTCCGTCCGAT
CCCTGGGTTGGGAAGATCCCCTGGAGAAGGAAGTGGCTACCCACACTCCA
GTGCTCTTGCCTGAGGCATTCAATGGACAGAGGAGCCTGGCAGGCTACAG
TTCATGAGGTTGCAAAGAGCTCTCTGCCTCATTTCGCACACACTTTTGGG
CATTAGTTAACAAGAACCCTGCTGCAACCAACCACATAAGTCGGCATTCG
CTATGAGGGCCTGGTAACCTCTCAGTGACCATGGGCTCTGAGTGTCATGT
CAGAATCTGTCTGCAGGGCAGGACAGTGCAGGGCGCCTCCAGAGATGAGA
CCCTTCTGAGCCGGGCGGTGTTCACACGGCAACACGACTACCTTTACACC
CAGTGAGCTGTCAAAGCAGCAGAACTCCTACAGCAGAGGATGTGCAGGTA
TTAGAGAGTCACGTCATCGGTACCACGTATACAATTAGACAGCCAGCGGG
AATGTGCTGGGGCTTCCCACGTGGCGCTCATGGTAAAGAACCCGCCTGCC
CACGCAAGAGACGGGGGCTCGATCCCTGGCTTGGGAAGAGCCCCTGGGCG
GAGGAAACGGCGACCCGCTCCACTACTGTTGCCTGGAGAAGCCCGTGGAC
AGAGGAGCCTGGCAGGCGACAGCCCACAGGAGTCGCAAAAGAGGCGGAAG
TGACGGAAGCGACTTTGTGCACACACACGCATGGGAATATGCTGTTGTGA
TGCAGGGAGCTCTGTAAACAACCTAGGGAGGAGATGGGCTGGGACGGAGC
TTCAAGAGGGAGGGGACATATTTATCACGTGTATACCTATGGCTGCTTCG
TGTTGATGGACGGCAGGGGACCTATGTGTACCTATGCAGACGCTGGTGTT
CATGGATGGTGGAAACCAACACGATACTGCACGTTAATTATTCTCCAATT
AAACAAACCAACCACATCTAAGGACTTCGATGGGAAAATTGGAGCAAGAA
GGCCTCCTGGGTGCAGAGTAAGCAAACGCTAGCCAAGGCCTGCAGGGCAC
CGTCCAGCGGACCCAGCCAGAGTCAGAGGACACTAGAGGAGCCCGCTGGA
CCCAGGCATCCCACACTCACTGATGGCGTGTGCTCCGTTCTCCTCCCCGT
TGACTGTGGCCTCGCCGTTCTTTGGTGGGTTCTGCTGGGAAGCGGCCGCG
GGCGCTGCCGTCGTGGCCGCTGCTGCTGCGGCTGCCGCCGCCGCTGCAGC
TGCCGCGGCCGCAGCCGCCTGCTGCTGGGCCAGCTTCTCCCGGAAGGCCT
GCTGCCGCGTCTGCACCACGTCCGGCATCACCGCGTCGATCAGGGACAGG
GACTCGATGGGGCGGCCGTCGAACACCGTGCCGTCCTGTGGACAGAGCCC
CGGGGAGGGGGGTGGGTCAGCTCCGGCTGGGCGTCTCCTGGGTTGGAAAC
GCAGGTGCCTGGGGCCCCGGGGAGGCGTGGGGGGCTGGGACCGGGAAGGG
GTGGCCCATAGACACCCAGCACTCGGTCAATAACAACCTGAACCCAGACG
TGAGGGGGTCACCCCAAGCCTCCTATTTCCCATCACAACCGGAGGGCTGC
AGTGGTCTCAATGGAAGGGGGCGATCGTGCTCCTCAGGGCAGGGGGGCAT
CTGGGAAAATCTGGAGGCGTGTCTGGTGGTCATGACTGCGGGGGGGTGCA
GCTGGCATGTAGTGGGGAGGGACCAGAGATGCCACCCAACATCCTGAAGT
GCACAGGATGCCCCCTCCGCTCTGAGAAGTCTCTGGCTCAAGCTGGAAAG
TCCTGCGCCCCCCCACCCCCAACAATCAGGCTCTGACTCATAGCGTATCC
TTCCATCTGATATTATCAAACGCATGTTCTGCCATGTGAAGTGACTTCTG
GGGACTCAGGATGCAGACAAATGCAGCCACGAAAYTGCTAACAACGGCAC
CGCTGAGTTACCACGCCTATTTCCTCAGCTTCCTCTCAGTTTCCTAGTTT
CCATCGGGGATGAGGAAAAAAAAAAAAAAATCTCCTACCCTTTTAGAAAA
CACAGGAGGGAGTAAGAGAGTCCTTTCCTGAGAAGGTGCTCTGATTATTA
GAGTCACAAGAAACTCCAGGCCTCTCGCTGTGCCTGCTAGATGTTCAGAC
AGGTTTTAGACCGTTTTTCCCTCATAAATCAGGAAGAACTGAAGGACGTA
CGTATCACCTACAAAGTGCTCGAATGAAACAGCACCCCAGCCGACTACCG
GATACTACACGATATTTGAAAATTAGSGAACAACAGAAAACAATGTAGAA
CGTGGCAAAAAGAAGCCACCCTCATCCCCTGATTATGCCCATGGAGTGGC
CAGTGTTGACCGGCTGGCCAGACGGCTTCTACATCACCTTCCTTCTTTGC
ATATTCATGCCCCAGACGGATGACGATTTTGCAAAAGGTAGCAACTGCGC
AGAGTAATAAAACGCTTCCTTGCCTAAACTCTAAACGCACACCATCTCAC
TCCTAACAACCGGAAAGCTAAGTGATCAATGAGACGACCTTGCAGTCTCC
TCTAGGCGATGTTGGACTTTGAATTGACAAGGGAGAGAAGGTATGGAAAT
GAAAGACGTGGAAACTGGGTGTGTGTGTGTGAAAGTCACTCAGTCATGTC
TGAGTTTTGCAACCCCTTCTCCAGGCCAGAATGCTGGAGTGGGTAGCTGT
TGCCCTCTCGAGGGGATCTGCCCAAGCCCACGATCAAACCCAGGTCCCCT
GCATTGCAGGTGGATGCTTTACTATCTGAGCCACCAGGGAAGCCCAAGAA
TACTGGAGTGGGTCACCTATCCCTTCTCCAGGGGATCTTCCTGACCCAGG
AATCAAACCTGGGTCTCCTGCATTGCAGGCGGATTCTTTACCAGCTGAAA
ATTCTTTGCCTTTACAAGGGAAGCCCATATGTGTGTGTGTGTGTGCCCAT
GTATATGTGTGTGTGTGTGTGGATGTGGATATGTGTCTGTATGTGTGCCT
GTGTGGGTATGTGTCCGTATACATATCTGTTACACGTGTGTGCGTTTGTG
TGTATCTGTGTGTGCGCAGGGAAATCCAATCACGGCGTCGCATAATCTGA
GAGCTGGGGTCCTGTACTCCTATCCCCACATGTGTTTTGTGGGAAAAAAA
AAAAGACTTAGATGGGGTCACGTTGCCAGCAAGAAGATCCCGGGCACCCT
GATGTACGAAAAGGAAAGAGGAGATTGCCTGCTGTTGGGCAGGAACGCCT
ACAGTATCCGCAGACAGCCAGGTGTTCTCTGAGATGCGGGGTTTTGGAAA
ACGACAGAGGAACGCATGTGAGAGAATGTGAGATACACTACTTGTGTGAT
TGTAAAAGCTTAAGGGAAAGTGGAGGTAAATGCTACGAAGAAAAGACAGG
CCTGCAAGGATGAGAAAGGGGAAGAGCAGGCGGAGGAAAGCGTGGAAGGG
TGCAGGGAGATGGGCTCGAGGGTGGTGTGATCGTGGGAGCAGACTTCTGG
TAGTGAAGGGGAGGGGGCGCATGGCCACTGAACGGGGTCAGCTGTGCTGG
ATGCGTTGGAACAGAAAACGGGGACTTGGGGCACAGCGGTGTGCTGGATG
GAGGTGGGGTGTGGGTAAATGGAGGCAGGACGAAAGAGAAAGGGTGGTAA
GTGCAGTTACAATCAGAGGGAGTCAGACTGAGACTCCGGGAAGACCCGGA
CGTCACAGGAGGCCCCGGGATCCTTAGAGGACAGTCCCCAGACAGAAGCA
AAGCAGGTGGGAAGGGGAGCTGTCTTGACCCCTCACCTTTTATCTTTAAA
AGGCTTCAGTTCAGTTCAGTTGCTCAGTTGTGTCTAGCTCTTTGCGACCC
CCTAGAATACAGCACGCCAGGCTTCCCTGTCCATCACCAACTCCCGGAGT
TCACTCAAACTCATGTCCATTGAGTCAGTGATGCCATCTAGCCATCTCAT
CCTCTGTCGTCCCCTTCTCCTCCTGCCTTCCGTCTTTCCCAGCATCAGTG
TCTTTATAAATGAGTAAGCTCTTCACATCAGGTGGCGAAAGTACTGGAGA
TCCAACTTCAGCATCAGTCATTCCAATAAACACCCAGGACTGATATCCTT
TAGGATGGACTGGTTGGATTTCCTTGCAGTCCAAGAGACTCTCAAGAATC
TTCTCCAACACCACAGCTCAAAAGCATCAATTCTTTGGCGCTCAACTTTC
CTTATGGTCGAACTGTCACATCCATACATGACTACTGGAAAATCCATAGC
TTTGACTAGATGAACCTTTGTTGACAAACCTTTGTCTCTGCTTTTTAATA
CGCTTTCTGGGTTGGTCATAGCTTTTCTTCCAAGGAGCAACAAGCACCTT
TTAATTTCATGACTGCAGTCACCATCTGCAGTGATTTTGGAGCCCCCCCA
AAATAAAGTTTCTCACTGTTTCCATTGTTTCCCCATCTATTTGCCATGAG
GTGATGGGACCAGATGTCATGATCTTAGTTTTCGGAATGCTGACTTTTAA
AAAATGTTACAATAACCCATTTATAAACTGCTATAAAATTGGCCCTGATA
TGCACATGCCCTGATCTATAAAGAGGGAGGGTATTTAAGCAAATCAGGAG
CAACTTAAATGTACACGTCTGGTCCCAACCTCCCGGAAGCAGCTGTGCAG
TGAGAGCTCTAACAAAGCCATCGCTTGAAATAACCCTCTTGAACTCAACA
GTACTCATCTCTCCGCTTCGATCTTCTGGGAGTGCCCTCGCTCAGGTTCA
CATAAAACACAGACTCTAGTGAAGTGACTCACAGGGTCCCTCTTCCACCG
AACTGCTGAGAGACATCCAGGAGGAAGGGTCATTTCCGTGCCCGAGGCTG
GCCCATGCCCATCGTGAGGCATGGCTGAGCCTTGAACGAGGCGGGTCTGA
CCTCCCAGGGTCCACTACACGCAGATGGACGTGTTTCAGCACGTTTCCCA
CTGTTTGCCCTCCATGGCGGGCGGAATCCGCAGGTACAGAATGGCTGACT
GTGGGACCTGAGCACCCCCTGTGGATTCCGAGAGACGACCGTATAAATCA
GCATCCTTTTCAATGCATCATGATGACCGCTTTACGGTGAACGGTGTCAG
ATTCTTGATCTCCAAAGAAGATTTAGCTTCGGGACCAGGGACCACGCTTG
ATCACTCAAGAGCTTTTGTGTAGCAGAAGTTTTATTAAAGTATAAAATCG
GACAGAGAAAGCTTCTGACACAGATATCAGAAGGGGGACGAAGAGTGTGC
CCCTCACTAGCATTGAGTTCAGTTGCTCCATTGTGTCAGACTCTTTGCAA
CCCCATGGACTGCAGCACACCAGGTCTCCCTGTCCATCAGCAACTCCAGG
AGTTTACTCAAACTCGTGTCCATTGAGTCGGTGATACCATGCAACCATCT
CATCCTCTGTCGTCCTCTTCTCCTCCTGTCCTCAATCTTTCCCAGCATCA
GAGTCTTTTCCAATGAGTCAGCTCTTTGCATCAGGTGGCCACAATATTGG
AGTTTTAGATTCAACATCAGTCCGTCCAATGAACACCCAGGACTGATCTC
CTTTAGGATGGACTGGTTGGATCTCCTTGTAGTCCAAGGGACTCTCAAGA
GTCTTCTCCAACTCCACAGTTCAAAACTATCCATTCTTCAGCACTCAGCT
TTCTTTATAAGTCCAACTCTCACATCCATACATGACCACTGGGAAAACCA
TAGCCTTAACTAGATGGAGTGTTACCAGTGTAAGCAAGGGAGCTATATCC
TTTCTCATTAATTATTACAATAAATCAAAAGAATGTCTCAAGTTTGTGAA
AACTTTACCAGACCAACTCACATAATTTACATTGTAAGATAACAGCATTA
GCCAGAAGGTTTTCAGGAAGGAGAAACTGTCCTCAAGCAGGAGATACACT
GTTGTTATATAATCCTTAGGACAGAGTTTAAAGTGAGCTGTTTATGTACT
CATCAGTTCAGGCTTAAACAAACAAACAAACAAACAAAAAACCATTTTAT
GTGACTAAGACTTAAGGAATGTCAAGGGGGAAAAAAAAAAAAAAAAGATG
TTTGTCCTTTCCTCTTCCTTGAGAATTTCAGGCCCCTATCTTCACCTTGA
GAACCCCAGACCCCTTTCTCCTTCTCCTCCTCCTTGGGGACTCCGGACTT
CTTATCAACCTGCCTAGGCATTGCCTCTCTCAGTAATACCACTGTGACCA
ACCAGTCCACAACCATCTATGGGCCTTGGGAGCTGGCTCCTTGGGTATCA
CAGCACTGGGATCCCCTACTGGAGAAAAGGATTTGGCACAAAGGGAGAGG
GGACCGTAATGCACTGGGAGAGGCCGTGGCAAACTCCCTTGCTAAGGTCT
TCTCAGAGTGCCCAGGCTCCCCAGGGTGGGGGCGTGCTTCAGGGCCCACA
TCCTAACCCCCCGCCTCCCCAACCCCTGGGAGGAGGGCCCACATTACCTC
GTTGATGCTGATTTCGGCCTCCACATACTGCAGGCCCTTCTGCAGGATGG
AGATGAGGGCGGCGGGCGGCACTAGGGTCCCATTGATGTTGGACTGGCTG
ATATGGCTCTCGATACCGAATGTGAACGCCGAGTGGGAAAAGCCTGCAGA
AGCACACGGGAGGTGGGGAGGGAGGGTGGGGTTCCATGAGCAGGCACCCC
CGGGGGGTCAGACGCACCTAACGAGTACAGGGCAGGGGGGTGCGGTCATG
CTCACAGGACGCCCAGCCTCTGGTGTGGGCGGTGTCCCAGAGGCAGACGG
CAGACCAAGGTGTGACTGCATGGAACAGCTGGTAAAAAAGCAGAGGCTTA
AAAAAAAAAAAAAAAGAAAGTGCCTTTCCAGTGCCATTATTATTATTTAA
ACAAAGAAACAACTTATTAAAAAAAAATTTTTTTTGGCTACCAAGTGCCA
CAAATGGGATCTTAGTTCCCGGACCAGGGATCGAACCCATGGCCCCACCG
CACCCCTGCACTAGGAGCTGAGGAGTCTTAACCACTGGACTGCCAGGGAA
GTTTCCCCCTCCCCCCATCCCCAACCCTTTCCAGCGTTATAAAAGCAAAA
CAGAGGTCACAGTCTGCCCCATTGTAGACGTTACAGAAGTCGGATCCCAG
GCTCTGTGTGCCTGGGTTCTGCATGGCTCCTTCCAGAAGCTCACGCTGAT
GTCTGCTGGCAGGAGCCCCCTCCCAGCATTCGGGGCAAATGAAGCTCACC
ACCAACAATGGGAAGAAACACGAGGTGGTTCTGTACCCATCGCGGGGTAA
CCTTGAGTCGCTTTACAGAACTGGGCATGTCCCTACCCGACCCTTGGCTA
AGGGCTGAAGGGTCCCCCCACACCCTGCAAAGCCCCTGCAGCACCCTCGT
CAAATGCCTCTGCACGAACCCACTGACAACACTGGGCAGGTGAAAGGTGG
TCACAGATAAGGCGTGCGCGGAGCTGCCTGGATGAAAATATGCAGGGAGA
AAGACTGGGCCGCTGACTACACGGAACAAATGAGGCGTGTGTCAAAGGAG
ACTGCTGTAACAAACGAGGCCGTGTCAATGAAGGAGGCTGCAGGGCCAGG
AAGGAGGTTTTTGGGGGCCTTCGGGTTGGGGAGGGAATGGTCTGAGGCAT
AAGTTTCCCTTCTGCTCTGTCACCTGTTGGGGGGTCCCGGGCACCCCACC
TCCATCACTTATGAACATCTTGCCATCAAGACTGGCTCATCCTATGTGCT
GGGGTGGGTAGAGGGGTTGGGGAGGGGGAGTCTCATGGGGGAACCTGCAC
ACTGCCTCCCCACCTCACTTTCCTAGGACACCCCCTTCATCAGCCGGATG
ATGGGCTGCCCGCCCCCAGGTCCCCCAGCCTCCATCCGGGTCAACAAATA
CAGCCAGCCTCTGCAATATACGGAACAGCCAGCAAGATGGCAATGTTACT
CCACGCAAGGAGAGCCCTTTCTCTTCCTCCACCCCTCCTCCTCATGGCCT
GATTATTAAGAATATATGCAATAGGCACTTGATTCATATTTATTTCCAGA
GTAATACTGCAGAGCCTGCTATTAACTTCCCGTGTTATTTCTTTACAAGC
CGGAACCACCTACATGAGACTGTGCAGGACAGCACCTTGGAGACCCTCTC
TCCAACACAGGGTCCCTGTGCTCCTGTTGCTGGAAGGGCATGAGCAGAGG
AAGGCCCTTTGCAAATCGGCCTTCTGTTTTTCCTTCTTACTTGTTCTCTC
TCTGGCCCTCCTTTGCCCTAACCACTGCTGCCACAACAGAAACATCCAGC
CCTGTCTTCACCAAAGACCTTATTCTCTGCATCTCCGCCACCACACACAG
AGCCTTAACCTCCAAGCTCTCTCATCCAGTTTCCGGCAAGGGAGCATGAG
AAAAACCCCAGGTCAGCAGACAGAGAAGGACAGAGTGGGTATCATCGGCT
GAGAGACACCCCAACCCCATGGACTCCCTACGTTCTCTGGTCCACCGTGC
CTTCTGTCTTGTAGAATTCTTCCCGGGTGACCGTCTGACCCACTGACTTT
TTCCATCTCCTTATCTGTGTCTCCACCAGCAGATCGGACAGGACGCTGGA
CTGAGAGTCATCTGCTTCCCTCCTGGTATCGTCACAAGGTCCAGAGCAGG
ACCACGCCCGCTGGGGTAATAACAAGACACCACCCGCCAGCCCCGACAGT
GCCCACAGCTGACCAGGTCTTGCTTCTAATTGAATAACGGTTCCTAATTC
ATGTAAGCCTTCCCACCACCACCTTCCCTCTCTGCTGACTCAGACGGTGA
AAGAATCTGCCTGCAATGTGGGAGGACCTGGGTTCGATCCCTGGGTTGGG
AACATTCCCTGGAGAAGGGAATGGCAACCCACTCCAGTATTCTTGCCTGG
AGAACCTCCACGGACATAGGAGCCATGGGGGTCGCAAAGAGTCGGACATG
ACTAAACGACTAGACTAAACGACTAAGCACGCACAGGACCGCGTGCAGTC
TCTCCCTTGTACCCTTCTCTTCTTTTTAGGAATCAGGTCCCGAGATCACC
CTTGGGGTCTGACACAGGCCACGCACTCGCTCCAAGCTTGTGGCTGGACA
CCCGTCTCTCAGCCCACCACAAAATTCCACCTGGAACCTTCTGCAGAGCA
TTCCCCCAGGGCATAGCGTGGGCTATGGCCAGGGCCCCCGACAAATAACA
TCTCAGCAACTGACGGAGCAGCGGTCGGAGAGAGGTCACACTTCCGGTGT
ATGTCTTTGTCCGTTCCAGCTGCTGTAAGAAAATCCCACAGCCTGCGTGG
TGTGAGCCACAGACATTTATTTCCCACAGTTCTGGAGGCTAAACTCCAAA
ATCAAGGCACTGGCACGTTCGGTGTCTGCTGAGAACCACTTTATGGACCA
GACACATGACTACGTTTTCCCTGTAACCTCAGGAGCAAGAAAGCTCTGTG
GGGCCTCTTACAAGGGCACTAATTGCATTCACGAAGGCCCATCCTCAGGA
CCTGATCAACACCTAAAGACTGAACTTCCAAATATTAACTGGGGATAGTT
GTTCAGTTGCTCATTTGTGTCTGACTCTTCGAACCCAAGGACCGCTGCAC
ACCAGGCTCCTCTGTCCTTCACCATCTCCCAGAGTTTGCTTAAACTCACG
TCCACTGAGTTAGTGATGCCATCCACCATCTCATCCACTGTTGCCTGCTT
CTCCTCCTGCCCTCAATCTTTCCCAGCAACAAGGTCTTTTCCAATGAGTC
AGTTCTTCACATCAAGTGGCCAAAATATTGGAGTTTCAGCATCAGTCCTT
CCAGCGAATACTCAGGGTTGATTTTCTTCAGGATGGACTGGATGGATCCC
CTTGCCATCCAAGGGACTCTCAAGAGTCGTCTCCAACACCACAGTTCAAA
AGCATCAGTTCTTCGGTGCTCAGCCTTCTTTATGGTCCAACTCTCATATC
CATACACGACTACGGGAAAAACCATGGCTTTGACTCTATGGACCCGGGTT
ACGTTTAAACATATGTATTGGGGGGAGGCTTGCAAAAATCAGTCTGCAGT
CACCTTGGAAATCTGCCATCTCTGGGAAGGATGCTACCCTTGGGGTGGCC
CAGCCTCCACCACCATCAGCGCCCACCCCTCCCAATCGGACTCCAGGGCT
GCTGAGCACATTCACTTGCAGTTTGCCGGTCCTATAGCTTCTGTGCCTTA
AGCGTGTTCAGCTTTCAGCACACCCCTTCTTCCACCCAACAAGACGTGGA
AACAAAGAACGGACTATCAGCGGGTAGAGACGGAAACGCTTGGGACTGGC
TCAGTGGCAGGCGTGCAAGTTGGTGAATAGGGGCAGAATAGAGAGCTTGG
TGGCACCTGGATGGCCGATCCAGGTGACAGCTCAGTGTGAGTGCCGTTGC
TGTGCACTGGGGCAGCCGCTGAAGCTCGGAGACCTGCCCACTACTTAGTA
AGAGGCATCCCCGATGAAGCACTCCTCAGGAAGGTTTCTCAACCGTCACA
TTTGAACCAAACTGTTCTTTGTTGTGAGGAGTGCTATGTCCTAGGCGTTA
CAAGATTTAGCAGCATCTATGATCTCTACACACTAGATGCTTGTTTTTTG
TTTGGGTTTTTTTTTTTTTTTTTGGCTGCCCTGCATGATCTTAGTTCCCA
AACCAGGGACCGAATCCATGGCCTTGGCAGTGGAGGTGTGGAGTTCTAAC
CACGGCACCAACAGGGACGTCTCATTACACACCAGGTGTTAGCAGCAAGC
CTTCCGTAGTCGTGACAACCGAAAACGTCTAACTAACCCCAGCACGGCAG
AGAACCAAAGGCCCACAGGAAGCACACCCCCCTTAGTGACACACTCCTCA
GGTCTACATCACGGACACATGTGCTTCATTTATAAACACCACGGGTGGAT
TGCTGTCACTGAAAAGCAAGCCACAGAATTCAGGTAACACATAGTCATGG
GGGGCGGGGGGAGCGGGTGGACAACCAGGTGAAGGAAGAGTATTTTTGAA
GAGAAAAAAGGGTATAATCATGACAAGATGGGAAAGGTTTGCATGTGCTT
AGTCACTCAGTCCGGAGAAGGCAATGGCACCCCACTCCAGTACTCTTGCC
TGGAAAATCCCGTGGACGGAGGAGCCTGGTATAGTGCAGTCCATGGGGTC
GCTAAGAGTCGGACACGACTGAGCGACTTCACTTTCCTGCATTGGAGAAG
GAAATGGCAACCCACTCCAGTGTTCTTGCGTGGAGAATCCCAGGGACGGG
GGAGCCTGGTGGGCTGCCGTCTATGGGGTCGCACAGAGTCGGACACAACT
AAAGTGACTTAGCAGCAGCAGTCACTCAGTCGTGTCCGACTTTTTGTGAC
CTCCATGGACTGTAGCCCACCAGTCCACGGGAATCCCTCTGTCCATGGGA
TTCTCCAGGAAAGTATAGTGGAGTGGGTTGCCATGCCCTCCTTTGGGGGA
TCTTCCCAGCCCAGGGAGCAAACCCATGTCTCCTGCATTACAGGCGGATT
CTTTACTGTCTAATCCACCAAGGGGAAGATTTATACTACATTTTTTATAA
GGCACAACAAGACCATCAAGAGTTAAGTATGCACCCCATCTGTTCACTGC
AGCATCATTCACAATAGTCAAGACATGCAAACAACCTAGATACCCATCAA
CAGGTGAACAGATAAAGACGACATGGTACATACAGAAAATGGAATATTAC
TGAGCCAAGAAAAAGAATGAAACAATGCCATTTGCAGCAATGTGGATGTA
ACTAGACATTATCATACTAAGTGAAGTGAGTCAGAGAAAGACAAAAATCA
AATGATAGCACTTATATATGGAGTCTCTTAAAAAAAAAAAAGCACTAATG
TACTTATTTACAAAACAGAAACAGACACCCATATTTAGAAAACAAACTTG
TGGTTACTAAAGGGGAAGGGAGGGAAGGGATAATTAGCAGTCTGGGATTA
ACAGAGACACAGCACCATACACAGAACAGATAACCAACAAGGACCTATGG
CATGGCACAGACAACTATACTTAATACCGTGCAATAACCTATAAAGTAAC
AGAAGTTTAAAAAGAATATAAAGAAAAATTAAATAAAAAAATTAAAAGAA
TATCAATTATATACATGTATGGGGCTTCCCTGGTGGCTCAGTTGGTAGAG
AAGCTGCCCGCAATGCGGGAGGACCTGGGTTTGATCCCTGGGTTCGGAAG
ATCCCCTGGAGAAGGGACAGGTCGCCCAGTCCAGTATACTGGCCTGGAGA
ATCCCATAGTCCATGGAGTCGAAAAGAGTTGGACACAACTGAGCGACTTT
CCCTTGTACTTGCTAAACAGCTGGAACTAACACACACTGCAGATCTACTA
TACTTCAATAGAAACAATACATTGAAAAAAAAATAAGATTTTGAAGAGCA
CTGTTCTTCCCATTTGGGGTGACAGGTGCTAGGAAAGCAGCGTGTGCATG
GATGTCCTCACCTACCGCTGCTGCTGCTGCTGCTGCTGCTGCTAAGTCAC
TTCAGTCGTGTCTGACTCTGTGCGACCCCATAGACGGCAGCCCACCAGGC
TCCCCCGTCCCTGGGATTCTCCAGGCAAGAACACTGGACTGGGTTGCCAT
TTCCTTCTCCAATGCATGAAAGTGAAAGTGAAAGTGAAGTCGCTCAGTCG
TAGCCAACTCTTAGCGACCCCATGGACTGCAGCCTACCAGGCTCCTCCAT
CCACAGGATTTTGCAGGCAAGAGTACTGGACTGGGGTGCCATTGCCTTCT
CTGCCTCACCCACTAGGCAACCCAAATAGATTGAAAACCAAGCACATGCT
TTGTTTGCTGCTAAACCACTAAGCCAGACCTCTTTGACAAACAACCTCCC
AAAGCATTCGAGCTGTGCAGTGAACCTTCCTGCCCAACTAATGTCGGCCT
GATTCATCAGCAGAACCTGCTAAAGAATCACTCACTGGCCCTTGGAAACA
CTGCCTATTCAGTGGAGAAAGGCAGACACGTAATATTCGGGCAGCTGTGA
GGCCGGTGGTCCACCTTCAGCTGAAACAAAGGCACCCGGGCTGGGAAGAT
CCCCTGGAGAGGGGAAAGGCTACCCACTCCAGTATTGGGGCCTGGAGAAT
CCCGTCGACTCTATAGTCCATGGGGTCACAAAGAGTTGCACATGACCGAG
CGTGTCCAACTCTTAACATTAAAGGCAGCATCAAAGGCACTTGTGTGAGG
CACTAAATGCAGTCTTTATTAAGGACTTCATAGACTTAATATTGGGAATA
CTGACAGCTATCAAAACCAGTGGGCACGATATGAAACTTATCCAGACCAC
AGATATGCAGCCCAGGGCCCATGCTGCCTGTGTTTATAAATCAAGTTTTA
TTGGCGTAGTCACTGCCCATGGGTTGATATGCTACCTGTGGCTGCTTTCA
CAGTGCAACGGCAGAGCAGAGTCGCTGCAAGAGACACAGTATGGGCCGAG
AGTGAAAGTACAAGTGTGCGTTGCTCAGTAGTGTCTGACTCTTTGCGACT
CCAAGGACTGTAGGCCGGCAGGCTCCTCTATCCATGGGGATTCTCCAGGC
AGGAATACTGGAGTGGGGTTGCCACGCCCTCCTCCAGATGGGCCACAAAG
TCAAAAATATCTAGGATATGCTCCTTTATAGGAAATGTGCGCCAACCTGA
CTATTCCGTGTCTATTGTATATGCACACTGATGTGTCCCTGTGTTCAGTA
GCTTAGCTGTGTCCACCTCTTTTCGACCCCACAGACTGTAGCCCGCCAGG
CTCCTCTGTCCATGGGATTCTCCCATGCAAGAATACTGCAGTGGGTCCCA
TTTCCTTCCCCAAGGTATCATCCTGACCCAAGGATCGAACCCGCATCTCT
GGCATCTCCTACACTGGCAGGAGGATTCTTTACCAACTGTGCCACGTTGG
AAGCCCATATATACACATATGTACAAATATATGCATGTATATCTACGTGT
ACCCACAGACATATGTGCAAATGTATATATATATTTATATACATATGGAC
CCGCAGACACAGACCACACGAAAGCAAGTGTCGCGGTCTACCCTGTATCC
ATTCTTCCCTTTCTTACTGCTATGGACTAAATGCCTGTCTCTCAACATTC
ATACACTGAAAATGCACTCCTTAAGGTGGTGGTGTCTACTTTTGAACTGT
GGTGCTGGAAAAGAGTCTTGAGAGTCTCTTGGACTGCAAGGAGATCCAAC
CAGTCCATCCTCAAGGAAATCAGTCCTGGGTGTTCATTGGAAGGACTGAT
GTTGAAGCTGAAGCTCCAATCCTTTGGCCACCTGATGCGAAGAGCTGACT
CACTTGAAAAGACCCTGATGCTGGGAAAGATTGTGGGCAGGAGGAGAAGG
GGATGACAGAGGATGAGATGGTTGGATGGCATCACTGACTCAATGGACAT
GGGTTTGGGTAAACTCTGGGAGCTGGTGATGGACAGGGAGGCCTGGAGTG
CTGTGGTTCGTGGGGTTGCAAAGAGTCGGACACGACTGAGTGACTGAACT
GAAAAAGTGGTGGTGTCAGGGAGGTGATTAGGACACGAGGGTGGGGTCCT
CACGAATAGGGTTCGTGTCCAAACATCGACACAGATGAACACTCAGTTCA
CGACTGTTATCTGCAGGACGACATACGACTTTCTTCCCACAACACCTTCC
TGGCCTGTAAGCAGCCCGCAGTGACCCCGCCAGCACCTGGGAATTCCTAC
CCTCCGTAATGGTGAGAAAAACTGTTGGTGTTTACGAGCCACTCAGCCTG
CAGCCTTTTGTCACAGCAGCCGAAAAGGCCCCAAGACGCGCCTCCTGCTC
TGTGTGATTGACAAGGTGACGGTACCCGTATCTGCTGTTCACCGAGCACT
TCCCTAAGTGTCAGCTGGCAATGTTAGAGTTTTCTAGGTGCTAGACGGGA
CCCAAGACCCGTATGTATGTGTACCCAGAGACAGGTTACCCATGGTAACC
TGGCTGATCTGACACGAGGAACCGAGGACTCTCTCTCCTTCTCAAAAGCA
GTGAAGAGTCTGCAGACCTGAGCGTGGCCTTGGAAGCCTCTCATTTCCTG
CGCTAAGAAGCTTGGCCATGTCTCACTATACCTTGCCAGGCTGCCTTTGC
CAGAAGCATATGTACCACTTTCCCACAGGACACCTCTGCTTAAGACTCTG
CTCCCTTGGTCTACAACGGTGGTTCGTGGTTGTGAAGTGTGGGCACTTCT
GTCGGCCAGAGGAAGACATGGGCAATATATGGAAATACTTTTGCTTGTCA
CCAGAGGCTGTGCCGCTGTGCGGTCGCTGAGTCTTTGAACAGCATTCCAC
GCGTCTGCTACAGTTTTTTTTTTTTACGAAGCGGGGCAACAGAGGATGAG
GTGGCTGGATGGCATCATCGACTCAAATGGACTCTGAGCAAACTCCGGGA
GATGGTGATGGACAGGGAGGCCTGGCGTGCTGCGGTCTGTGGGGTCACAG
AGATTTGGACACGACTGAGCGACTGAACAATGACAACTTTCTATTCGAGG
AGAGAACACACTTCACCAGCTTGGGCCTGCCCTCGTTGGCTTCCTTCTCT
GACCAACAAACAGGTGGAATTAGGTGATCGCCAAGGTCCCTTTCAATTCT
AAAATTCTATACAGAGGAAGGAACGGCCTCATTGTCCCAGTTGAACCCAC
AAGATCTTGATTTACGGAGGTGAGTGAGTGACGACACACAGAGATTAAGG
CCACCAAGAACCCGTGACTTGCAGTTCCTGTGGGAAGGGGCCATGCCACG
CTGCCAGGGATACAAGAAAGCTTATCGCATCAGGGGCCACATCACCGAGA
AACAGACAGGACCTAGACTACAGCTTTATGGAAACGAGGGTGAGCAAGCA
GCTCAGTGGGAATGTTGCCCACTGGACAGACGACAGAGTTAAGACTGTGT
GCTTGGGAGGTCTGTATCATGATTTTCACGAGAGCCACCTCTGAGAAAGA
GAGAGCGAGCCTACCGGCTCTGGCCATCAGTCCAACCCTACAATAATGAA
GGCCAAGCAGCCACTGGTTGGCACACTCATCCATTCCAACTGGCTATGCG
GATGGAAGCCCAGTTCAAAAATCACTGCAGATGGTGACTGCAGCCATGAA
ATGAAAAGACGCTTGCTCCTTGGAAGAAAAGCCATGACAAAACTAGACAG
CGTATGAAAAACCAGAGATGGTACTTTGCCAACAGAGGTCTCAATAGTCA
AAAGCTGTGATTTTTCCAACAGTCGTGTATGGATGTGAGAGCTGGACAAT
AAAAAAAGGCTGAGCACCAAACAACTGATGCTTTCGAACTGTGTCGCTGG
AGAAGATTCTTGAGAGTCCCTTGGACTGAAAGGAGATCCAACCAGTCCAT
CCTAGAGGAGATCAGTTCTGAATATCCACTGGAAGGACTGATGCTGAAGC
TGAAGCGCCAATCCTTTGGCCACCTGATGCAAAGAGCAAACTCACTGGAA
ATGACCCTGATGCTGGGGGAAGATTGAAGGCGGGAGGAGAAGGGGAAGAT
AGAGGATGAGATGGTTGGATGGCATCACCGACTCAAAGGACATGAGTTTG
AGCAAGCTCCGGGAGTTGGTGAAGGACAGGGAAGCCTGGCGTGCTGCGGT
CCATGGGGTCGCCGAGAGTCGGTCACGACTGAACGACTGAACAACAATCG
GGAATGGCACCTAACAATGGGATCATCTCTGGAAACAAAAGCGTGGGCTG
CTACGGTACATGCGGTGTTTCCTTGCTTCTGGAGCATCCCTGGGCGCAGA
GCAGTCAGCACTCTCTGTCCATTCCCCTCCGTCCATTCTCCCTGGATCAG
TTTTCTGCCACAGAACCGCAGCGGCCTGCAACACGGCCCGCGTCCCTCTT
GGTGTGGGGGACCAGCAGACTCGGGTGTGGGGGCGTTGGCGAGAGTAGAG
GTAACAAATAAAAAGGAAGAGAGGCTGAGAGCAGTGAAGCGGGTGCTGTT
GCTGTTCGGTCACTAAGCTGTGTCTGACTCCCTGTGACCCGGTGGACTGT
TACCCACCAGGCTCCTCTGTCCATGGGATCTCCCAGACAGGGATACCGGA
GTGGGTTGCCATTTCTTTCTCCAGGGGATCTTCCCAACCCAGGGATCGAA
TCCAAGACTCCTATGCTGGCAGGTGGATTCTTTACCACTGAGCCCAGTTA
AGTGCTGAATCACTGAAGTTGCTCAGTCGTGCCCCAGCTCTTTGCGACCC
CATGGACTGCAGCCTACCAGGCTCCTCCATCCATGGAATTTTCCAGGCAA
GAGTACTGGAGTGGGTTGCCATTTCCTTCTCCAGGGAATCTTTCCAACCC
AGGGATCGAACCCAGGTCTCCCACATTACAGGCAGACGCTTCACCGTCTG
AGCCACCAGGGAAACCCAGTTAAGTGGCAGCTGGGTCCAGGAAATCAAAG
AGAAGAAAACACACACACACACATGCTGTAAACAGGCAAGGCGGGAGAAG
AAGATACCATCCCCTCATCTTTGGGGGCTAATTTATCATCAGTTCACTTC
TTCTTTTTGGACGTGCTGCATTTGTCTCGCCAGAGGATTACCTTTTCTGC
CCGGGAAGCCACACAAAACCGTTAACCCCAGTTTCATTCATGGTCAACAG
CACAAATATTCACTGAGTTGTTTTTGACTCTGCAATCTGCAATCACTGCA
CACGTGTGCATCGACTTTATTGAATCTAAACCAACTCCCCCGCCCCAAGA
CCCAGCCCCCACTTTCTGTCCTAATTCTCTCCTATCTTGCTGGGGAATAA
CTTGAGCACAGGCAGGCTGCTCTCAGGGCTGCAGACCTACTCCCACTGAC
CTGCCGTAACATGGAATTTGGTATAAAATGGAACTGCTTGGAAGGCGCCC
AAAAGACATAGCAGGAGGACCTCGTTAAAAATAGGGTTTTGCTTCTAACA
TCTGGCCTGGACCCCAGGGGTGGTGGCTGAGTGCGGGCCTGCCAAGATGT
TTAATATCCCCCAAACCTGGGAGGGCTCCAGGAGGAGAAGACATTAGTCA
GGTTATAAATGCAGGGTGCTATTTCCTCCCGGGGCCGACCAACTGTGAGG
GGAGGCAGCTCGCCCACGAGGGGCGTCGGGGCACGCTCCCCCAGACCCCA
GCCCTGGCTTCCAGCCGCCACACAGAGAAGCAAGGTTTGGGGCCAGCCCC
TTTTGGCCCCATCACCCCAAGAGCCCTTGAACCCTCATAAAGTTTGGACT
CGCTATTTTGTTAATTGGGGAATTCACCATGACCGCCAACACGGAGATTT
ACGATGGATGACACCGTAAAAATTACAATAATAGAAATATACACACATAC
ACACACACACATGACTTAGGGGGAGGAGAAACAGAGAAAGCAGTTTGGAA
GAGAGAGGGAACAGTCTTATCACTAGATGCTTGATGCAAGTGCTGTTCTT
ACCCGGGGGTAATTTCCTCCAGTCCCTCTTACCTCTGTAAATTGATTTTT
CACACGCTTAATTCTTGTGGTCCTAGAATTTTACACCCCTGTTCTTTCTT
AACCTAACAGAGTATCAGCACTTCTCCCAGTGACTCATTAGTCCCGGCCA
CCATTCTGCCTGAGATGGTAAAGAGTTTGCCCCCAAGGCAGGAGACGCGG
GTTGGATCCCTGGGTCGGGAAGATCCTAGAGGTGGACACGGCAACACTCT
CCAGTAATCCTGCCTGGAGACTCCTATGGAAAAGGAAGCCTGGGGGGCTT
ATAGTCCATGGGGTCGCAAAGAGTCACTCCAAAGGGCCCACTGAAGCGAC
TTAGCATTCACACCATTCCGGTGGAGTCTCAGTTTTCACAGAGAGTCACC
CCTCATGTGCATGGCCTTCAGATTTCATTTTCAAAAACTAGCCAACGGGC
TTCCCGTCGGACACCCCGGCCTTCACCTGCTGCCCGTGACCCAGAGTCCC
TGTACTTCCTTCCCCTTCCTCCCATCCTGCATCCTTGGTACTTCTTGCCA
AGATGCGTCTTCACCCCTCAACAGTGTAACCGGCTTTTGCTGAGCCTTGT
GACATTAACGCAGGCCCTTGATGATGTTGCTTGAAATGCTCTGCTTCCTT
TTTCCCCTGAAGAGTTCTAGTTCAAATGCCTCCTTCTGTCTGCACCCCTC
CCCTGCTCCCCTGTGAACACCGCCAACCTCACATTTAGCAGCCCTTGTAT
CAAGACGCCTGTTTATTCCCTAGGTGGGGAGAAAAAGACTTCCTCCTCTA
GTGTGTCTCCTTCCAACCCTAGCACTAGAGCAGTGCCACCACTCAACAGG
GCCAGCGAGAGACATTTGGTAGAGCTGAAAATCCTGACAACTGCTTAATC
GGGGCACATCAGATTCCTGGAGAGTGCCTGTACCTCTGATGGTACCTGAA
ACAATTTTACAAAGCACAGGGCGTACATATTTTATTTTATTATGATTTTT
TAAGATATTTTAAAATCTAACAATTATTGTTTTAATTGTGCATTATGCAA
ACACAATAGTAGATGTATTTGAAAATTTAGTGAAAAGTTAACTGCTTCAA
TTTTAAGGAAAAATGTTAGTAATCATTAACAAAATACTTTTAGGTGTATG
CAGATATGGCAAAATGATGTCTCTGGTACAAGAGAAACAAGGATGGGAAA
TACATGTATAGACAAGGGAGGACTACGGACATGTTCAAAGGACAAGGAAG
TAATAAATACTCAGGATACAAACASTGTGTCCARGTTCCGTGGACAGGAA
GAAACACATGTATTGTATGGGCAGTCTCAGACAAGAGTCTGCAAGCYGTA
TCCTCTGGGCCAAATCTGGCCCCTGGCTTGTTATTATAAATAAAGTTTTA
TTGAAACACAGCTACTCTCAATGGTCYATGTACTTTTGATGGCTGCTTTT
GGACCACAAACACAGAAATGACGGTTGCGATAGAGACAAAAACAATACCT
TTTAAAGTAAGATTCTGCAAACTGATCCAGGGGAGAGACCTAAATTATGG
GACTACAGGTCCTCAGGAAAGCGGTTTTTGATCCATCGGCTTGTATAACC
CTAAGATGCAAACCTAATGAGAATATTGGAAAAGGAACATCACTGCCTAG
CTTCCCTCAGTAAAACAGACATGAACATACCAGACCAGAGAATTAACAAA
ACGGAATTCAGTGAAGAGGACAAAATGTCCAGGCCACATGGGATTTATTT
CAGGAATGCAGACTTAATGCTGAGAAAGAAATCCACCATCCCAGCACGTT
AAGAGTGAAGAAAAATCATTTAAGTGGAAGATTCTGGAGCCAGAGGGTGT
GCTCTGAATTCTAGCTCTGTGACTTCCTGGCAGCATGACCTGGGCAAGCC
GCTATCTTCTCAACACTTTGGGTCACTCACCTGTCAACGATGGAGGCAGC
TGTATCTACTTCTTGAAAATAAAATGAAAGCGTTAGTTGCTCAGTCGTGT
CCAACTCTTTGCAACCCCATGGGCTGTAGCCTGCCAGGCTCCTCTGTCTG
TGAGATTCTCCAGGCAAGAATACTGGAGTGGGTTGCCATGCCCTCCCCTC
CAGGGGATCTTCCCGACCCAGGGATCGAACCTGGGTCTCCTACACTGCAG
GCAGACTCTTTTTTATCATCTGAGCCACCAGGGAAGCGCCATCTCCATCT
TAGGGGGAAACAGTTTGACTCATCAAGAACTGAGAATCATGCCCAGCACA
CAGCTCATGCTCAATAAATGGGAGCTACAGTCATTCAGACAAAGCAGCGG
AACAGATGGAAACCCTGATAAGATATAGGGAGTGCCTGGCAGACAGAGAC
GCGAGGATGAGAGGACTCGGCATGGGGGCGGTCGGAAAGATACCGTGGAC
GAGAAATAGATGACAGCGATTCTGACATTTCCACGTGAAACCTGGATGAG
GTTACGGGGGTCAGAGGAAGTTCTTCTTTGCAGGGGAGGAACTGTGGTCA
GGAAATGGCAGTGCTCTCAGTGTGGGATCATGCTGAAGGATGCTTGGGAA
ATACACAACTAACAGCAGTACTGAAGGCAACGGAGAGGCTGGAGGTGGCC
CCCCCCAGACTGGGGGATCTGAGGAGACGCTGGGGAGAGACCACCCACTC
CCACCCCCAGACAGGGGATGAGATGCGAGTTAAGGATGGAAACCTACACT
TGCAGAAAGAGCACCCCACTGCCCGCAGACCTGGATGCGGGAGGGAAAGG
AAAAAGGATGTCACCTTTGGGATAGTGGCATTTTAGACGTGGACGGAGGA
GACGTCACTAGGGCTACAGACAGACACTCGGGAGAAAAAAAGGACAGTGT
TGTGTCTGACAGTCCAGATGACCGGCTCGCCGAGTCTGGAAGGAAGAAAG
GGAGCTCTGATTGGCAGAATGAAGTACATCAAAGCTGGACATTTTGAAGG
TCACATTAGAATTTCAGAGCAGGCAGATCTTAAATGTGATGGTGTATGAT
CTTTGGAAACAGGCACCGTGCTGAGCACGGTTAATTCATCTCAGCTAAAA
AAGGAAACGGGAACCATCTGASGCTTTTTCATATCGTCCCCACCCTTTAA
AGGAGGACCAATGGCCTAGGAAGTCGTAAGGACTTCTTCAACAGCACCTG
ACCTTCCTCTGATGGCCATACGCAGGGAAATAATTTGGCTATATTGAGTA
AGACACACGCTGAAACAAGTGTAACCTACACGCAGGCTTCCTGGGTGGCT
CAAGTGGTAAAGAACCCACCTGCCAAGGCAGGAGACCCAGGAGACGCAGG
TTCAATYCCTGAGTTGGGAAGATTCCCTGCAGAAGGAAGTGGCAACCCAC
TCCAGTATTCTCGCTCACAAAATCCCAAGGACAGAGGAACCTGGGGGGAG
CTATAGTCCACGGGATCGCGCAAGAATCAGACGTGGCTGGGCGACTAAAC
AACAACTTACAAACACTCGGCCGATTTGCAAGTGCTTTTCTAGCGCATTG
CTTTCTCCCAATTCTCTGACAAGTCAACAAGCACAGGAATGGCTAAGCTT
GAATCTGAGCTTTGAACACATAGACGACGGAGGGCAGCAAACTCAGAAAA
GATGGCAGAACCGCAACCGCATGGGAAACCGGGATGGCTGAGAATCTCGG
GGATGTGTCGGGAGCAACCGCACCACGGTTCCGTTTTGCTACGCTTCAGT
GGGTTATCATCACACCCAAACAGCAGTTCAGGAAGACACTTTTGTTTTTG
AATGAAGAGGAGCTCGTTGGCAACGGGCACCACTCGAAGAGGGTGTGGGA
TGAGATGACCCCAACCCAGTGCAGGGTCTTTCGAAAGCCCTAAGGGGCCT
AAGGGGAGATGGCCCCCCGCAGACTGAGCTCTGTCCTGCAAAGGAAGCGT
GGGCAAGCTTTGCTGCGGGAGGTGTGTGCAGCCCTGCCCCCACAAAGCTC
TCCTGGGGGGGTGCTTTCTGTGTGAGAGGCGTGGGTATCACATTCTCCAG
TTACTTACCCACCCTTACACTTTTTACAGTGAGTTTTCAAACATGAGGTA
AAAACACACAAGTGCACACATGACCGTTACACCACCACACACCATGGTGT
CCACCTGGAGGGGACTGGGGCTGGGCGGACAGCCTGGGTCTGCCCAGGTG
CTCCTGCCCCTTCGACAAGAGCTGCAGGTCCACAAAGAAGCCTGGATGGC
AAAGCTTGGGTGGCTAACAACTCAGCCCCTTTGCCATCGGACATGCTGCT
GGGAGAACCAGGTGTCATCACACTGCTCAGCCAGGAGGGGACACCCCCAA
GTCTGTGCCTGGTTCTCCTGGACTGGACCCCATGCACCTTTGCCCCTGGC
TGACTGTAACCTGTCTCTTTTTAACTTGGGAGGGGGGTGGGGGTTGTTCC
CAGGTGCTGCTAGGGGTAAAGAACCCTCCTGCCAATGCAGGAGACTTAAG
GGATGCGGGTTAGATCCCTGGGTGGGGAGGATCCCCTGGAGGAACGCAAG
GCAGCCCACTCCTGGAGAATTCTTGCCTGGAGAATTCCCCCAGTCCCTAG
GGTTGCCAGAGATGCCGAGAGTCACTTGAAGTGACTTGGCACCTACCTAC
GGATTACATCTCAATGGAGAAAAACTGAAGCTATCAATTCAAACAAAGGG
TTCTCTTGGATACTGTGTAAACAAGAAAGCTGAACAAACAGAATATACAC
CCAACTGTATACGATTAAGAGGAATGTAACAAAAATGGAGCATCGACTTT
GATTAACGTGTTGGTCTTTTCGATTGACTTTCTTGAGACGTAAGTGCCAC
CTAACAAAGTGTAAGTTTGGGGTATGCACTGTGATGATCTGATACATGGA
TATACGCTGTGGAAGAACTACACAATACGCTCAGCTCACATATGCAGCAC
CTCACATGATTAACCTTTACTTACTTCACTGAGAACATTTCCAGGATCTT
AGCAACCGTCAAGTACCCAATATACCACTATGAAGTAGGTTAAAATCCGT
ATCTTTTTGTTGTTATAATCCGTATCTTTTTGTTGTAATCCACTGCGAGT
GTAACAGTTACTAAATTCTGTGAAAGTCGCTCAGTCCTATCCAACACTTG
GCGACCCCATGGACTATACCATGTAATTCTCCAGGCAGGAATCCTGGAGT
GGGTAGCCTATCCCTTATCCAGGGGATCTTACCAACCCAGGGATTGAACC
CAGGTCTTCCGCATTGCACGCAGATTCTTTGTTTACCAGCTGAGCCACAA
CGGAAGCCCAAGAATACTGGAGTGGGTAGCCTATCCCTTCTCCAGTGGAT
CTTCCCCGCCCAGGAATCGAACCGGGGGCTCCTGCATTGCAGGCAGATTC
TTTAGCAACTGAGCTACCAGGGAAGCCCAAATTCTGTGACTCCTTCTAGC
AAATCATAGAGCCTAGGGGTAGTCTAGGAGACAGGGATACATCACCAAAG
AACTGGAAACAAAGAAATGAGACATTTCTGTTTCACCTGACTCATTTCCT
TGCTCCCTCCCTCCCTTTCCGTCCTCACAGCCTGCAGGATTGTAGATCCC
CAACTGGGGATCGAACCCCAGTCCCAGCAGTGAAAACACCAAGTCCTAGC
CACTGGACCACCAGGGATTTCCCTCACCTGCTTTTAAATTACTTTTATCC
TGTGCTCGTGCGGACAAACCATTCAGGTATTCCTTACTTATTATCTGATG
AGTATTTTCCTGTATGCTTTCTAAAGCAGAGGCTACCACTGCCTCACAGA
AGTGCTTGCCTTTACCGTTTCATAAGGATCCTCTTTTTAAATGGGGACAA
CATTAGCAATGTAAAAATGCCACCGCAGTCAGAGAAGTATTTTTAGCAAC
TCAGCTGAACAATGAGCCCTGAACCTGGGTTTAAATTAAGCTAGCTGAAC
CATTAAAATGCCTTCCCGAATCCGGGGTGGGGGAAACGTGTCTAGCATAA
GAAGGCAGTCCAAGCTCCCAGGAAACACAGTGGAGCCTACAGGGTGATCT
GTCAGCCGCTAGAATTAGAGGGCAATGCTGCTGAATCATTTCTGGAGACG
CAATTATTCACGAATGTTAAGAGTAACACCCTTTTCCTGGTTATCTAATC
TCCTCACCGGGAGTCCAGAAAAAACCAAAACCCAAAGAAAAACAAAACCC
AGATTGAAGACAGCTACTCCCAATTATTTTATTATCTGCTGGAGAACAGT
ATTGGCAGAAGTTCCCCCCTTCTTCAAGGGGAAAAATACTCTGGAAAAAT
TTTATAACAACAGGGACCGCTAGGACCACTGTTAAGAGTTTCATTTGCTT
TCTGCAGAAAGAATCAAAATGCAGCCCAAGAAGACGAAACAGAAGGGAGA
AATGAGAATCTCGTTACAACTCCACTTTGCAATAAAAACTAACTGAAGAA
ACACATCTCACAACTGAAGGATGTGTTATCAGTGGTTCTCAACTAGGGGT
GGTTTTGCCCTCAGAGGAACATTTGGTAAAGGTCAGAGACACCTTTGGTT
GTCACCAGGGAGGGCGAGGGGAGCAGGGCGTTCCTGTCATGCAGTGGGTG
GAGTCCAGGGATGCGGTCTTAAGAACCTACAATACAGCGTAGCCACCCAG
CCCATACATCAACAGTGCTGAGGTTCAGGAAGCCAGGCCTCTGAGAAAGG
TCGAGCGTCTTCAGATAATGCATGCTGTTCTGACCATTCAGAGTCCCTCC
AAGAGATTCAGTTCAGTTGCTCAGTCGTGTCCGACTCTTTGCGACCCCAT
GAACTGCAGAACACCAGGCCTCCCTGTCTATCACCAACTCTCGGAGTTCA
CTCAAACTCATGTCCATCGCGTCGGTGATGCCATCCAACCATCTCATCCT
CTGTTGTCCCCTTCTCCTCCTGCCCTCAATCTTTTCCCGCATCAGGGTCT
TTTCAAATGAGCCAGTTCTTCGAGTCAGGTGGCAAAAGGACTGAAGTTTC
AGCTTCGGCATCAGTCCTTCCAATGAATATTCAGGACTGATTTCCTTTAG
GATGGACTGGTTGGATCTCCTTGCAGTCCAAGGGACTCTCAAGAGTCCTC
TCCAACACCACAGCTCAAAAGCATCAATGCAAGAGATTAAGACCCTCAAA
CTCTGCCCACCTAGGAGAAATCTACAAGTGAAGACCATGAAGGGGATACT
GAAAACCTGGCAAGACACAGGACCAGGAACGCAGAAGAACACTGAAGAAC
ACTATTAAAATATTGATTGATTTTCGGCTGTGATGGGTCTTCGGTGCTGT
GGGCGGGCTTTGCTCTGGTTGTGGCCAGCAGTGGCTACTCTCGAGTTGCA
GTGCCTGAGCTCCTCATTGCAGTGGCTTCTTTTGTTGCAAGAGCATGGGT
TCGAGGGGCACGTGGGCTCAGGTCGTTTCAGCTCATGGGCTTAGTGGCTC
TCCAGCACTGGCATCTTTCCACACCAGAGATCGAACCGGGGTCCCCTGAA
CTGGGGGGTGGATTCCTATCCATGATGCCACCAGGGCAAGTCCCTGAATA
TGGACTTTTATCCTTCTGCTAACGATACAGCCTGTCCAACGCCTATGGTG
CCAATCAAATGCAGAACAGCTTCTCACGACAATGTTCCAGGAGGAACGGG
CCAGGTGAGCCTGGCCTATGATGCACACTTGAGTCTGCTGAATTACCAAC
GAAGCAAAAATCTGGCTCTGGATACACAACCACCACTGTTCACCTTCAGA
TGACTCTATTATTTTTCTGTTGCCAAAAGCCCCAGAATGGCTGTGTGGCT
ATCCGCTTCTGAGCTAACATGGTAACACTCGAACCCATCATACAAAAGAC
ACTCCATCAGAACCGAGGCCTGAGCCTTGGCTAACGACCTGCAAAATCTT
AATTAAAATGTCTCGGCTGCATTTGTATTACTGACCACCTTCTCTGTCTC
AGTCATAGGCAAAGCTCTGCAGACCATGGAAGCGTAAAGTCATGATTTCT
CTGGTCTTTTTTATTTTAGTAATTTCAAGATGATCATAAGCTGTGAAAAC
AGTGGAGTCATTTCACCTGGCCACCTTCAACAGTGACATCTTCTCTCACC
GTAGGACAGTGTCAAAAGCAGGGAATGACTTTCAGGTGATGGGTCACCTG
ACTGCAAACCTTCAGCTTCCCTCCTTTCAGATCGGTGTGTGAGCATCACC
TAGGCACACAGGGTCTATGTGAAAGCTACTCCATCATGTCTGACTCTTTG
TGACCCCGTGGACTGTAGCCCACCAGGCTCCTTTGTCCATGGGATTCTCC
AGGCAAGAAGACTGCAGTGGGTTGCCTTTCTTTCTCCAGGGAATCTTCTC
CACCCCAGGATCGAACCCAGGTCTCCTGCATTGGCAGGCAGTTTTTTTTT
TTTGTTTAACCACTGAGCCACCTGCTGCTGCTGCTGCTGCGTTGCTTCAG
TAGTGTCCGACTCTGTGCGACCCCACAGACAGCAGTCAACCAGGCTTCCT
GTCCCTGGGATTCTCCAGGCAACAACACTGGAGTGGGTTGCCATTTCCTT
CTCCAATGTATGAAAGTGAAAAGTGAAAGTGAAGTCACTCAGTCGTGTCC
GACTCTAGCGACCCCATGAACTGCAGCCCACCAGGGTCCTCCGTCCACGG
GATTTTCCAGGCAAAAGTACTGGAGTGGGGTGCCATTGCCTTCTCCGACT
GAGCCACCTGGGAAGCACTTATTCAGTGAATTACACAATGTGAGATCTTC
CAGGGCCAGGGATCAAACCTGTGCCACCTGTATCGGCAGGTGGATTTTTA
ATCCCTGGACTACCAATATGCATAATTTTTACACTGCTCAGGAACATTTC
TGCAGAGTCATACCTCTGTGCGTTCAGAACAGAACCATCCTTTGGCTCTG
TGCCTGGACTTGCCTGCGGAGAGAAAATGCCACCTAAATCTTACTCGGAA
TCTGATGTGAGGCGGGGCAAGAAAGGGCATATGTGTGTGCTACCTGGTCA
GGAAATCTTTCTAAGCATTCTCCTAACAGAAGCTCCACGAAATGCATGAA
AAAAATGCCATCTACCTTCCATTTAAAACCTCAAGAGTCTGGTTATGTAA
CATGAATTCACAAGGATAATACAGACCACAGAGCCATGTGCGTATTATGT
AAATCAAAGGAAACATGCCTGATTCTGCCTGGCCAGTCACATGAGTGCAG
ATATGGGCTTGAAACCAGTCCATCCTAAAGGAAATCAACCCTGACTACTT
ACTGGAAGGACTGATACTGAAGCTGAAGCGCCAACACTTTGACCACTTGA
TGTGAAGAGCAGACTCACTGGAAAAGACCCTGATGCTGGGAAAGATGGAA
GGCTGGAGGAGAAGGGGACGACAGAGGATGAGATGGTTGGATGGCATCAC
TGACTCAATGGACATGAGTTTGAGTAAGCTCCGGGAGTCAGCGATGGACA
GGAAGGCCTGGTGTGCTGCAGTCCATGGGGTCCGCAAAGAGCTGGACACG
ACTGAGCGACTGAACCACAACAGAAAATAGCTCCTGTCCCATGTTGTAGG
CGCTTCTGTTTGAAAACCACCAGCGCTCCTCGCAGAAGACAGCAGGTATG
GCTGTGCTAAGGTAGGCAACAGATATGCATAATAGATTAGCCTCCTGACC
ATGAACCACGACAAAGCAGGAAGCATACTTCACTGTATAAGGAGTTTGCC
AATCTTATTCAAAGGTGTTTATTTAATTTTTTAGTCTTTACTCAATGTCT
CGCCATGTTGCTTCTGTTCTACGTTCTGGGGTTTTTGGCCTCGAGGCACA
TGGCATCTTAGCTCCCCAGCCAGGGATCGCACCCACAACCCCCACATTGG
AAGGTGAAGGCTTAACCACTGGAGCACCAGGGAAGGCCCTAGAAGGTCTT
TTGTTGTTGTTGTTCAGTCGCTAAGTCGAACCCAAGACTCTTTTGAGACG
CCCATGGACCGCAGCACGCCAGGCGCGAATGTTGTTAAATCCCCACAATA
AGCAGAGAAATGCACCATCGTGTTGATTTCTGATATATGCGAAACGAATT
TCTGCCATCTGCGCCGTAACCAAAGGCTGCTTTTATTGTCTCAAGTGTTT
CAAATGACAGCCACTGGGGACACCCAGCCTGGTAACACCCGACAAAACAT
TCGGGAAACCCAATCTCTCACTCGTGTTTCTGGTTCGCTGTCAGAGCTAC
CACCACAGTCGTCAAATGGTTTCGATTAAAGGTTCTATCTGTTTTTCACG
GTGTGCAGACGTTGGTCCCAATGCTAGAGGGAGGGGAAATATGCCCCCCA
AGCCATGAACACCTTGGGTGAATCTGCATGAATACCCATGCAAATCAATG
CACGTGCAGTCTTGGCTCCTGTCTTCCCAGGTGGGGCAATGGTAATGCGC
CTGACTGCCCGTGCAGGAGACTCAGAGGACATGGGTTCGATCCCTGGGTG
GCAAAGATCCCCTGGAGGAGGGCATGGCAAAACACTCCAGTATTCTTGCC
TGGGAAGTCCCATGGACAGAGTAGCCCGGCGGGCTACAGTGCATGGGGGT
GCAAAGAGTCAGACATGACTTAGGGACTAAGCATATCAAGTTCAGTTCAA
TCACTCAGTCACATCTGACTCTTTGCGACCCCATGGACTGCAGCACACCA
GGCTTCCCTGTCCATCACCAACTCCCGGAGTTACTCAAACTCATGTCCAT
CAGGTTGGTGATGCCATCCAAACATCTCATCCTCTGTCGTCCCCTTCTCC
TCCCACCTTCGATCTTTCCCAGCATCCGGGTCTTTCCCCATGAGTCAACT
CTTCGCATCAGGTGGCCAAAGGATGGGAGTTTAAGCTTCAACATCAGTCC
TTGCAGTGCATACTCAGGACTGATCTCCTTTAGGATGGACTGGTTGGATC
TCCTTGCAGTCCAAGGGACTCTCAAGAGTCTTCTCCAACACCACAGTTCA
AAAGCACCAATTCTTCGGCACTCAGCTTTCTTTATAAAGAAACTCCAGTT
TCTTTATAAAGTTGGATGTCCAAGTCTCACATCCATCCGTGACTACTGGA
AACACCATAGCTTTGACTAGATGGACCTTTGTTGGCAAAGTAATGTCTCT
GCTTTTTAATACGCTGTCTAGGTTGGTCACAGCTTTTCTTCCAAGGAGCA
AACGTCTTTTAATTTCATGGCTGCAGTATCTACCTGCTGTCTACCTAGCA
TTCCACCCAGCGCCTCCCACCCAGTTTTCTGCAGAAGGGTGCACTGGCTC
AGGAGAAGCAGGCGGGCACGTGTGGACCCTGTCCCACTCAGCGGATCCAT
GGGAACCACGACCCCAGTAACAGGTTCACAGAGGCTAATTCCAAACCCTG
AGAGGAAGCGTTAGCATTTCATCAGTATTGTGCAGATATATCTGCAGGAG
ACCAATGTCTTTTTACCTATTTTCCAATATTCCCTGTGCCCCAGGCACTG
ATTATCATTAGCTCTGTGGCAGGAATGAAATCTATAATATAAAACCACCA
GCGGCTGTGAATTGCCTTGCTGTTGAGGGAGACCAAAGCAGACGCGGTAG
ATTCTGATAAACTGAACCCAGTTTTGTCACCTGGCCCAGCAGTGCGCCTC
CCAGACAGCTGCCCAAGAAAAAGGAAAACCCCAGACCACAAAAGCCTATG
TGCAAATGCCCCCAGGAGCCTTAGTCGTAGCTTGGAAACCACCCAAATGC
TCTTCCACAGGACAAACGACTAACCGCCCTGCATCCATAAAACAGAATAC
TGGGCCACGCTTAGTCTCTTAAGTCATGTCCAACTCTTTGCGACCCCATG
GACCATAGCCTGCCAGGCTCCTCTGTCCATAGGATGCTCCAGGCAAGAAT
ACTGGAGTGGGTTGCCATGCCCTCCTCCAGGGGATCTTCCCAACCCAGGG
ATTGAACCTGCATCTCCTGCATTGGCAGGCGAGTTCTTTACCACTGTGGA
AGCCCATAGGAAATAGAAGACCATGTAGCATATAAAGGAAGTACTACTGA
AGTATGCACCAGCGTTAGCCAACCTCAGATGCACTGAGTCAGAAAAGCCA
GACTCTCAAGGGTACACGCTGCAGAGATTCACTTCTCAGTCACAATCGCA
GAGGCAACACTCCAGTGCCAGGAAGCAGACCGGTGGCACCAGCAGCTGCG
ATGGGGTGAGGAGGAGACTACAAAGGGGGAGTTTTTGGTGGAACCATTCC
ATATCTTGTTTGTGCAGGTGTTTGTATCACTCTGAACCTCTGTATTCTAA
AAAGGGTCAATTTTCTGGCGTGAAAATGATACTCTATGTAAAAAAAAAAA
AAAATGGTAGAGATGAAGGTGCAGAGGGATGGAGAAGCTGGACACTGACC
ATGAAGGGAACTGCCAGGCTGGGTGGAAGAGACGCTCTGCCTCAATCAGC
TTCTTACAAATCGTCAGGATGACAAAAGCGACCACATGATTAACCCCCAA
GAGGCAGAGGCCGATGGACTCCTAGCCCTCCAAAGCGGACTGCCGCAACT
CATGCAAAACATAAAAATTAAAAGCACAACTAAGCCACTTTGCTGCTGGG
GCTGCTCAAAATCTCCAGCTGAACTGAAAAAAAAAAAAAAAAAGCCTTGC
ACACTAACCGATCTTCTTTATCTGGATGCCAGGAGGAGCAACCATAAACA
ACAGCATCCAGACAGCACCTTCCACAGGCACGGCCCTTGCTTTAGACAAC
AGTGACGCCATGAAATCCCCTCTGCTTTAGAGGCTGTGGAATCCACGGCA
GGGCGGAGGGAGGCCAGAGGGGAACCCTAGATGATGCGAATTCTGCCTCT
TGAGGGCAGAAAAGGAAGCCCCTTTTCAAGATACTTTTCCTGAGTTTGCA
GAGATGTGATGTGCATTTTGTCGTAACACTTTCAGGAAAGTGTTGCTTCT
GCACTGCGCCTCCCAGCTATCTGGTGGGGTGGGGTGCATGAGGAATCAAT
GTTCTGATGGGCACCGTTTTCCCTGCAGCCCTGAACATCCTGACCGGCAC
ATGTGCTGCCCGACTGCACCAGCCCAGCCCCTGCGGTGGCAGAGAACTTG
TGCCTTGAGGTGGGCTGGAATATTCTGGGTCTACCCGCTGCCTGGTTGTA
CAGCCAACAGCAGACCCCCAGAGATCCAGAGGTCTGCCTCCCTGCAGGGT
CCTCCCCACCTGCCATCTCCCAACCTCCTACACACACACCTTCTACACAC
ATAGCCTGGGGGGTGACACGACTAAAGACCTAGGAACTTTCAAACCCCTA
GTGGAGAAGCCACACTGGGAGGAGCTGAGTGAGTCACCTAGCGTGACAGA
GAATTTATGGATGATGGGTTTGATGCTGGTAAAGGAGAGACAGCACAGAT
ACTTGATCAGGAGAGTCCTTCCTTCTCCTTCCTGCCTTCTTTTCCTTCCC
CTTCCTCCCCCTTCCTCCCCCCTTCCTCCCTGTCCCTTCCTTCCTCTTCT
TTTCTTCATTCCCTCTTCTTCCTTCCCCTTCTGCCCTCTTCCTTCCCTTC
CCTTCCATTTCCTTCCCCTCCTTTCAGCTAGAAAGGAGACGTTATGAGGC
CAAGAACATGCTCTGTCCACTTGATGGCTGCTCAGTAAATTCCCTGGTGG
CTCAGTCAGTAAAGACTTTGCCTGCAGTGCAGGAGGCCTGGGTTTGATCC
CTGGGTCAGGAAGATCCCCTGGAGAAGGGAAAGGCTACCCACTCCAGTAT
TCTTTCCTGGAGAATCCCCATGGCCAGAGGAGCCTGGTGGGCTACAGCCT
ATGTGGTTGCAAAACATCAGACACAGCTGAGCAACTAAACCACTACCTAA
CCAAAGCGTGAGGATTTGAGATAAATGTTAAACAATCCCACAATGAACAT
TTAACAACATTAGGCATTTCTTCTTTGGAGTATGCCCTCGTTGGGGTACC
AGAGGCAGTTCCCGGACGCACACAGTCTTCAGAGGAACACACAGTCATCT
CTTAACTTCTCACTCGATCTATGGGATTCTACTCAAGGCTCTGAAAAATT
AGGACAGCCGCTCAGCAGATGCTACACACAATGGGCATTGATGGAATACA
CTGTGTCTCCCAAGGCTCCTTTCTCAGTCGCCTGGTTACAGAAAGTTTCT
CAACAAATGTGAGACAAATGTAAACAAATCCTAAAAAAGGAAGTCTCGCT
TTTCTGGGGGGAGGGATTAGACATTTCTGTGTTTATTCAAAGAGGCCAGC
AACGCTCTTGCCCCGGGGGGCCCTGGGGACTGCCCACCGGGAGAAACCTT
TCTCTAGGTTACACAAAAGTTTTCTTTCCCAAGTCTCTATTTACCTTCAT
GTTCACAGAGGGATGGCAATTTCACCTTAAGATTGTTTACACATTTGGAC
TGCACTTTTTCAGCCTATTTTTGTGCAAGTGTTTCTAAAGAGAAAGAATC
CTGAATTCCAAGCTGGAAGTCTTCAGGGAGGCAGGGTTTGGCACGTGGAA
TTTTCATCTGCAGACATCTCTGGGAATCTATGAGATCAGTGCTGGGAGGA
CACTTGGACGCCAGGTGAGAGATTTCCTGGTTAATTTCCGAGGTTAAGTT
ATCAGAGCCGTGGGTTGATCTCCCTAGATTGACGAGGGATGAGGTGGTAC
ATATTTTGTTCCGCTCATCATTTTTTTCATAGGAAATATCTAGCAAGAGC
TAATCCAAATGGYCWWATGWACATGTAGAAAGAAGGTGTACTCTTCATGA
ATAATGATAGCTAGGGTGGACAGCAGAAACAGAKACCTCAGGCTTGCGTG
GGGAGAGGTTTATTTATAACATGTGGCATGCCCCACGTATTTTATCCTTT
TTGTTCGAGGATCAGAATCGAGCCATTTGGGTCGCCTGCAAGACTGGCCC
ATCTCACCCTTCTGGGCTGATTCTGAAAGAGTCTGGACCCAGGGACTCAC
ACTTGGATCAACCTCAATGATAGCATGTGCGGAAGTGGGATGTGAGAACT
GGCTTCTTTCTCGAATAATCAATTTGCAGACTACAGAAAATGTGACACAA
AGTGAATCAAGCTTCAATATGCACAGAACCGACTGTTATAGCTTGAACTG
TGTCCGCCACAACGGCATAGGCTGGTAGTCCTAGCCCACAGCWCGGCTCA
GAATGTGACGGCGCTTGGAAGAGGGTCCCTGCAGATGWCATCTGTTAAGA
CGAGGTTGTGCTGGAATACCGTGAGCCCCGTAATCCACTATGACTGTGTC
TTTACRAAAAGGAGAAGTCCKACAAATGTGGACAGAGAGTGACACGCACA
CAGGGAGAATGCCTCGTGAAGGKGAAGGCAGGTCTCGGGGGACAAASCGC
CTGTGAWGCAAGGAKTGCCAWAGACGGTCCAKCCGAASCTGGCAKACAGG
CCGGAGCAKACMCYCGTGAGGCAGACAGTCKCGGGAAGAGAGCAAGCCAG
CTKGCACCCTGCTTTGGACTGCTGTCCTCCACGGCGCTGGRAYAGTGCAT
TTTTGTTCAAGCCGCCCCACTGAGTTACCCCACACCAATTCTGTACACGG
ATCTGTAACCAGCCCACAAACGCTCGCTCTGTGGCCGGGACCAAGAACAC
CAAACACCCCAGCCCCATCCATCTATATATATATACACACACATACATAC
ACACACACACACACATACATATACATACACACACTATATGTATACACCAG
CCCGCAAATGCCCTCCCTATGGTGGGGACTGAGAACATCCAATCACCCCA
CTCCCCATGTATATACACCATATGTATACATTAGCCCACAAATGCCCTGC
CTATGGTGGGGGCTGAGAACATCCAATCMCCCCACTCCCCGTGTATACAT
ACCATATGTATACACCAGCCCATAAATATCCTCTCTATGGTGAGGGCTAA
GAACACCAATCACTCTACCCTCTCCCTATATATACATACATATATATACA
CACACACACACACACACATACATATATACCASACCACAAAAGCCCTTCCT
ATGGTGGGGGCCAAGAATATAAAATCACCCCACCTTCATATATATACACC
AGCCCATAAGTATCCTCCCTATGGTGGGCATCAAGAACACCAAACACCCC
ACCTCCATATACACACACACACACACACACACACACACACACAGACACAC
ACATTTATATGTTTGCTTGTGTGTTTAGCTGCCCAGCTGTGTCCGAGATG
CTTTGCGACCCCATGGACTGCAGTCCGCCTGGCACCTCTGTCCATGGAAT
TCCCCAGATAAGAATAGTGGAGTGGGTTGCCATGCCCTCCTCCAGGGGAT
CTTCCTGACCCAGGAATGGAACTGGGGTCTCCTCCACTGCAGGTGGATTC
TTTACCAGCTGAGCTACCAGGGAAGTCCCCATATGTATACACCAGCCCAC
AGATGCCCTCCCTATGGTGGGGGCTGAGAACACTCAGATCACCCCACCCC
TCATGTATATACACCACATGTATACAGCAGCCCATAAGCACCCTCCCTAT
GGTGGGAGCCAAGGACACCCTATCGCCCCACCCTATAATATATATACCAT
ACGTACACACCAGCCCACAGATGCCCTCCCTATGGTGGGAGCTGAGAACA
TGAAATCACCCCACCCCCCACGTAGATACACCAGCCCATAAACACCCTCC
CTATGGTGGGAGCCAAGGACACCCTATCGCCCCACCCTATAAGATATATA
CCATACATACACACCAGCCCACAGACGCCCTCCCTATGGTGGGGGCTGAG
AACGTGAAATCACCCCACCCCCCACGTATATACACCAGCCCATAAACACC
CTCCCTATGGTGGGAGCCAAGGACACACTATCGCCCCACCCTATAATATA
TATACCATACATACACACCAGCCCACAGATGCCCTCCCTATGGTGGGAGC
TGAGAACATGAAATCACCCCACCCCCCACGTATATACACCAGCCCATAAA
CAGCCTCCCTATGGTGGGAGCCAAGGACACCCTATCGCCCCACCCTATAA
TATATATACCATACATACACACCAGCCCACAGATGCCCTCCCTATGGTGG
GAGCTGAGAACATGAAATCACCCCACCCCCCACGTATATACACCAGCCCA
TAAACAGCCTCCCTATGGTGSGAGGTGAGAACATGAAATCACCCCACCCC
CCAGGTATATACACCATATGTGTACCCCAGCCCATAAACACCCCTCCCCT
ATGGTCGGGACCAAGAACACCAACCGCCCCACCCCCATGTAGATGCACCA
TACATACACACCAGACCACAAACGCCCTCCCGAGGATGGGGACCCAGAAC
ACCAAATCCTCCCCGCCCCTCACCGCATCTATCACAACTGCTCTTGATCA
AAATGGGCTTCCCCTGTGGCTCAGCTGGTAAAGAATCTGCGTGCAGTGCG
GGAGACCTGGGTTGAATCCCTTGGTGGGCAAGATCCCCCTGGAGAAGGGA
AAGGCCACCCACTCCAGTATTCTGGCCTGGAAAATCCCATGGAAAGAGGA
GCCTGGTCAGCTATAGGTGTTCGGGTTACAAAGACTCAGACTGAGTGACT
AACCCTTGATTTCACTTTATTAGAGGTACTTCCTTGGTGGCTTAAGACGG
TAAAGAATCTGCTTGCAATTTAAAGAGACCTGGGTTCAATTCCTCAGCTC
GGGAAGATCCCCTGGAGAAGCGAAAGACCACCCATTCCAGTATTCTGGCC
TGGAGAATCCCATGGACCGCATAGTCCACGGGGTTGCAAAGAGTCGAACA
GCACTGAGTGACTTCACTTTCCCTTCACTTGATCTTCATTCCAGGCTGGC
TTCAGGTGAACAGAAATGTCAGCACAAACAAGAACACATTCCCTCATCCC
GCCCCAAGAGCAGGACGGCCCAGCCCCTTCTCCGCCTCCCTTCAGTCCTC
TGAATGAGGGTGTCCGGAGCTCTCTCTTCCCTATAGACCGGGTTTTCCAT
CCGTGGTTGCTGGCCCTGCAGGTGGCGTGTTGGAAGGTGTTTTTTTTGCC
GCTCCGGGCAGGAAGCCGGCTTCTTACCTGACTCCTGGAGATATCGGTAC
ACCAGGAAGTTCACCTCGTCACTGGTAATGCTCATCTTAGCCTCACCTCG
CGGGGATGAGGTCTTTACGAAGCAGCAGCCACCACCCTCTGTCGGAAAAG
GCAAGAGGAGAAAGCGGAGAGACGTTTCAGACACTGCCTGCGTGTTCCTC
CTCAACCGCATTTCTTTCGGAAAATAACTGATACATGTAATACTCAGGAG
GTAACTAATGCCCCATTCCTGCCATACGTTCTGGTAACATAAAGGTTTTA
TAATTATCTAAACATATATACACATACAGATGAAAAATTTCCCCTGTATT
TCCTGGTGACATAAAGCTTTTATAATTACCTAAACATACAAACATACAAG
TGAAAAGTATCCCCCAAAACACGAGCTGATGCGTTTTATAATGCTGGCAT
GGACTCATCTATAAACCACATTAACGGAGACAAAATCAGGGGCACCAAAA
ATGAGAACAAACACCGCGACTTGATTCAGTGAAGATCCTGCAGCGGTTGG
TACCGAGCTTCAATGGAACCCCGAGACAGAAGGTGGCTGGTGGCACCGAC
TGGCCCTTCCCTGGACAGACGAGGAAGATTAGAGTCCTGTCGACACCCCC
AGGTTGGGTGACCACGTGGCTCTGGTCACACCTGTGGGTCTGCAGCACAG
ATGACTCACAGCAACCTCCCACGCATCCAAGTTCCATGCTGAACCTGACG
GTCACCGAACCGACAGGGTGCATGTGGCCCCGACATACACAGCGAGGAGC
TGAGTCATCGACAAGGAGATATGACTTCTATTAGGAGGCGGGGAGTCAGC
AAGCAGCCCCGTGGACAGAGTGCAACGACAGCATTCGGTTCTGCGGCAGA
CTGGCCAAGACCATGGCATCTTCAAGGATGAACCAAGTGCTTCTCGGGAC
CCAGGGGCAGGAAGACCAGCCTGTGTGCCCGGACTGTGGACTGAGATGGC
CTGTGTCATCGGCCAAGCAGGATTCTTACAGAGCTCAGAATGCTTGCATG
TGCTAAGTCGCTTCAGTCACGTCCGACCCTCTGCGACCCCCATGGACTAT
ATGTAGCCCCAGCCAGGCTCCTCCGTCCCTGGGATTCTCCAGGCAAGGAC
ACTGGAGTGGGTTGCCATGACCTCCTCCAGGGAAATGCTTGTGGTGACTG
CAATGAAGTTAGGGGAACCCCAAGGCAATGGTTCAGACCTCCAAGGACTG
ATGGGTTGGAGGTAAGGCAATCATGTCCTGAGGGCACCAAGAACAGAGAA
CATGGCACTCGGAGCGTGGGGGGGCTTGAGGCTGCGCCAGGAGGCCTGAT
GCTGACTGCACCTTCATCCCTAAGGAGCTGTGGACCACAGGCCTCAGTTT
TCTACCTATGCCAGCTGCGGATGCTAAGAACCGCTGCCCTCCCCCTCCCC
CAGGAAGGTGGTGAGGATGACATGCTTCTACACACACTTCCAGACCAGGG
CCCTGAGCACCACAGAGACACAGGAGGGCTCTTAATCCTACTCCGCCCCA
CTGCAAGCATCCTCAAAGCAGAGAGACCACCACCGCCCCCAGGAACACGC
CTCCAAAAACAATAACAAAAATGGGTTCTTGATTAGTGACATGGTGTTTA
TTTTCTTGCCTGACCCAGTTTTGATTCATGCATGTGTGTGCTCAGTCGTG
CCTGGCTCTTTGCAACCCCCATGGACTGTGGCCCGCCAGGCTCCTCTGTC
CATGGGATTTCCCAGGCAAGAATACCGGAGCAGGTTGCCATTCCCTTCTC
CAGAGTTCTCATCGGAGGCACCTCCCATTAGGTAGATCTGAGAATCTTTG
TGGTTGTGACTGGGCAGAAAGGAAATGACCGTCTTCACTGTGTGGTAAAA
ACCACACATTCCCTCACTGTTCTGGTTTCAGAAGAAAAAATCAGTGTGAG
CTGATGACACGTCTGGAGACTTCCTGCTTAACATGAGTGTTCCAACATAA
ATAACCACTGGGGGGCTGAAATCCATCAGGAAGCTGAAGGGAAGATTWTT
TASGGTATTTTTCCTTTTTTAATAACTTACTTTCCAGGTGGCAAAAAACT
CTTAATAACCACAGTCATAAAACAGTTCAAAATTCAGTTGCAAGAGAAGT
CGTGTCTATGATGTTATACTTAAAATGGAGAACCAACAGAACCTGCTGTC
CAGCACAGGGAACTCTGCTCAATCCTCTGTAATAACCTTATGGTCACCAG
GGGGAAGGATGGGGGAAGGGACAGTTAGGGAGTTTGGGATGGACATGGAC
ACACTGCTGTATTTAACATGGAGAACCAGCAAGGACCTGCTGTCCAGCAC
TGGGAACTCTGCTCAATGCTCTGTAATAACCTTATGGTCACCAGGGGGAA
GGATGGGGGAAGGGATAGTCAGGGAGTCTGGGATGGACATGGACACACTG
CTGTGTTTAACATGGAGAACCAACAAGGACCTGCTGGACAGCACAGGGAA
CTCTGCTCAATGTCATGTGGCAGCCTGGATGGGAGGGGAGTTTGGGGGAT
AAGGGATACATTTAATGTACGGCTGAGTCCTTTTGCCTTCTACCAGCAAC
TACGGCAATGTTGTTAATCGGCTGCATCGCAATACCAAGTACAAAGTTAA
AAAAATAGTTTTTTTTTTTTTTTTTTTTTAAAGGGAGTGGGAAATGGTGT
CTAAGGGTTTGGCTGTATTGCGTGTACAGTATTTAACATGCTCTCATACT
CCAGGAAGGGACAGGACCGCTCCCCAAACAACACAATCTGACCTGCTCTG
TGATGATCTTAAAGGATGTAATGACCACAGAGCCACTTGAGCGTCAGCTC
TGGGAGATGAAGACAGACAGGGCGAGTGAGTTTCCCTGCCCACGGGCGGG
GAGCCTGGTTCAGAAACAGCGATGGAACAAGCACACAGTCACTCCAGGGT
GGGGAGCGTGGTGTTCAGTGTGATATCATCCATGGCAGAAGTTTCTCTCT
TAGTAAATCGGTGCCGTTAGRACACAGTCTTATAACATCCTCTCATGCGG
CCCCTTCCGCCGTTCTCCAGTGATGGAAGAAAAAGTCGGTCTGAGGTCTT
GTCTCTGCTATGAGTCTGCTGGCCTGGACACTGGAAGAATGCATCGCGTA
AGGTGATCTTCTGGACAAGGCCCTGGGACTTGATTCCACGCTGAGTAAGG
ATGCAAAGCCAGAGCTGGAAAATCTGCTATAAATGACCTCATTGCAGCAA
GGCACTTTCACAAATTCTCTGCCAACTGCAACATAGTAGGACTCACCTCC
TGGCTTCCCTTCTCCATAATACTGGGGGTAGTTTAGCCGATCAGTCGTGT
CCAACCCTATGTGACCCCATGGAATGTAGCCCACCAGGCTCCTCTGACCA
TGGGATTCTCCAGGCAAGAATACTGGAGTGGGCTGCCATTTCGTCCTCCA
GGGGATYTTCCCGACTCAGAGAGTGAACCTGGGTCTCCTCTGCTGCAGGT
GGATTCTTTACCGACTGAGCCACCAGGGAAGTCCATAACATTGCTCTCTT
TTCAAATTGAAACTGTGTTTTCCAGTGGCCAGTTATGAGACTAGAAGCTT
AATGTGTGTGTCAACATTTACATATAAATGCTCTTGTACACACTGCTGGA
GACTAAGGTGGGGAGGAAGCAAGCCTGTTTCATCACATTACACACCTGCT
CAGCTTGCATTAACTGAAACGAAGCCGCCACGTAACTGCTGGAAAACGAC
TGCAAACTTTCGTTGTTGTCCAAAGACAGTAATGAGTACTATTCAATAAA
TGATGGCTTTTATGGTTGGACTTCAGTTATTGCAGAATGTACATACAGAA
TAGATTGGACAGAAAACAAGAAAATGTCAACAGTGGTTTCACCTTCTTTT
CTTCCCCCACAGACTTATCTTCATTTTACTAAAGAGTATTTGCATGCATA
TTATCCTACAGTGAGAAAAACAGTCCCCCCAAAGGTTAAGATTTTTCACT
AATTGTATAGTTATATTTATTTACTTGGGAGCCCAATGGTCCAAAATTAT
AAACATAATCAAAGAACTATTCTTTCCTCACCAGAGTTCACATGCCCAGC
AGTGGCATGCATGCGTGCTAGCTTGCTTCAGCGTGTCCGATTCTTTGCAA
CCCTATTACCTCAGCCCGCCAGGCTCCTCTGTCCATGGGATTCTCCAGGC
AAGAATGCTAGAGCGGGCGGCCCTGTTCTCCTCCAGGGGATCTTCCCAAC
CCAGGGACTGAAACCACGTCTCTGACGTCTCCTGCACTGGCAGGCGGGTT
CTTAACCACCAGTGCCACCATCTGAATAAGGGAATGCAGTAACCTAGTAA
GACTGCAGTCCTCAAAAGGATTTTCATGACCGGCAATGACATCCATGATA
CAGTGCTACGTGAAAAAAAAAAAAGGAGGCTCCAAATGGAATGCGTGTGT
GCCTAAGTTTTAGTTTGTACACACACACAAGGCCTCCCCCGGTGGCTCAG
TGGGTAAGCAATCTGCTCCCCAACGAAGGAGACCCAGGTTCGATCCCTGA
GTGGGAAAGATCCCGTGGAGAAAGAAATAGCAATCCATTCCAGTATTCTT
GCCTAGAGAATTCCATGGACAGAGGAGCCTGGCAGGCTACAGTCCATGGG
GTTGCAAATAGTCAGACACGACTTAGCAAGTAAACCACACAAACCACACA
CACACACACACGTACAAATAATAGATACCCTCTAAAATGTAAACAGTAAA
ATAAAATGCACCAGTCTCTCCAGGGCTGGGACGATGCTGGACTTAAATCC
GTGTACGTGTGGCGTGCCTTTTCGGTATTCCCCTAATTTCCTCCTACAAA
CGTGAACTGCAGTCACATTCATTAGGATGTAATTAAAATCAGGCAGCGTC
TTCAGAAATCAGTATGAGAACAACCCAGCTAAGGAGGAGAGAAATTGGCA
GGGTTGAGACAGCAAGGAGAAGGGAGCCTCCTCGGGTTAGAAAAATAATG
ACACGCTTCACAGAAATCAAAAAACACCCCCAGGGTGAGGCCTTCAGAAA
GGGGCCTTTCTGAGCCTATGTGCCGGCCCGGATTCATTAGTGGCTGGTTG
GTTAGCTTTGTTCCGACTCTCTGCACACAGGCACGAAGCATGGAGACGCG
AGAGGACGGGACGTCCTGGCCCAGACGTGCGGCCAAGACCACGTGTCCCC
CTCCCAAGCAGCCATGGGTCCCCGTCTCGAACCCTCTGAGCTGCACACCC
TGCCCACCCCACACTTCTCTGCCCCATCGGGAGGCAGGTCCAGAGGGAAA
CCTTCCTAAAAAAGTTCACCCCGATGATAGCAAGCCACTGCTAAGTAAGG
TCTGCGTGGCAGAGAGACAGACACACACGTAAGTCCGACAGCACCGTTAT
ATTTTACAGCCTTCCGTCCACGAGGTGAAATGCAAGCTCAGGTGCCCAAG
CTTTCGCAGCTAAGTGCCTCGGGAAATACAGACAACTTCAATGTAAAAAT
GCCCTTTCCCTTCTTTGCTGGAGGACGTGGGTCTGCCAGCTAAGGCTTCC
GATTAGTCCGTAAATTAATTTACAGACTTTAATTATCTGAGACCGAGGGG
CTGGAATGACAGTCTCTGCACTAGGCCCTCTGACAAATCACAGGAATATA
CACAGGCTGGTTACGAAAGCTCTCAGACAAAGTGATTTTCAGCCACATAT
GCAAGATTTATCTTGGCCTGGATGATTCCCTTTTCTGTGGTAGGTGGAGT
CCTAAGTGAGGTCATCGACTCCTATAATGTGGTCTCCAGAGTTTTGAAAA
AACAAACAAGCAAAAAACCCTGTGGCTTTGTCTGTAAATATAAAGGCAGG
CGATGCTTTGCCCTCTGAGTCAGCCTGTGTCTCAAAAACCTTGGTGCTAA
AATAGGGAGATATTGAGGGCAGGAGGAGAAGGGGGCGACAGAGGATGAGA
TGGCTGGATGGCATCACCGACTCAATGGACATGAGTCTGAGTAAACTCCG
GGAGTCGGCGATAGACAGGGAGGCCTGGCGTGCTGCAGTCCATGGGGTCT
GCAAAGAGTTGACACGACTGAGCAACCGAACAACAAAAACAAAAGTATAC
TGTGATGCTTCAGCTTTAAATGAGCATGGTGACCCATGCCTCTATTCTTG
CCTGGGAAATCCCTCGGACAGAGGAGCCTGGTGGGCTACAGTCCATGCGG
TCTCCAAGAGTTGGACATGAGTGAGCACGCATGCACACACAACAACCACA
CACCACATAAAAGAAAAATACTCAAAGCACCGTGAAGCCGAGTATCCCAA
GAGTGCCCTTTGACACTGAAATCAGCAAGCAGAAGGCATTCAGTTTTGTC
ATCTGACGCTCGGCAGGTGAAATTCCCACAGCCAGAGGAGTGCGCAGATC
AAAACACAGAAAGAAATTCTGATGCGAACAGGGAGGGGTCGTCCGTCTCG
GTACTGCGGACACTCAGGACCACATGGATCTCTGTGACGGGTTCCCCGAC
GCCCTGGAGGATGACAAACAACATCCCTGGTCTCCACCCACTGGGTGTCA
GGAGCAAACATCTTCCTCAAAGGTCTCCAGACGTGATCAAATGTCCCCTG
GAGAAGGCTGGTTCCAGACGTCCAGAGCCACACTGCCCATGGAAGGCCCT
GTAACTCCTCAAGTCCATGAGCAGCTGCAGGCTGTTGGCCAAGCAACCAC
AACTTAGTAATAAAGTGTCAGTCACTTGACCGTGCCTGACTCTTTACGAC
CCCACGGACTGTAGCCCGCCAGGTTCCTCTGTCCGTGTGGATTCTCCAGG
CAAGGAGAATCATGTTCATGGAGTGCGGTTGCCATGCCCTCCTCCAGGGG
AATCGTCCCAACCCCTGGATCGTACCCGTGTCTCCTGCCCTGCAGTCGGA
TTCTTTATCATCTGAGCCAGCAGGGAAACCACAATGTGGACGCTGTCTTA
AGCTCGCCATGGACCTCGCAGAGCTGCATGTCAGAGGCCTTCTACTCGAA
GGTCACGGTCAGAAGACGGAGAAATAAAATGGGTGTCTTTCTTGAGGTCA
AGGCTGGCGCACCGCAGCCCACAGGCAGATTCACACGTCGCCCACCTTCC
GTAAGGCTTAGGAGCTATCCACGGTGTCTCTGTTTCTAAATGGCTGCAAG
GAAAATCAAAACAATCCTATTTTGCCACCCCTGAAATTGCGATGACATTG
GAATTCCAGCGCCCATCGGCGAAGTATTACTGATGTCACTCAGCCACACT
CGTTCATCTGTGTATGATCAGTAGCGGTTCTCACAGCGCAAAGGCAGAGG
AAAGCAACGGAGACCCCGGGGCCCACAAAGCATCAGCTTTCAACATCTAC
TCTCTGGCCCTTGATGGAAAACGTCTGCCAACCTCACTCCCCTGAAGATT
CTCCCCTCTCTACATTAGAACACGCCCGTCTTGTTGTTTTCCAGCCGAAA
TTCCTGTCTGGGAACCTGAAGGTGATGCCCTAAGAATGTGGATGCTCCTC
GGGCATAACCCACTGGTGGCTCTGAAATGAAGAGTCAAGAAGACACCACA
GCCATCATCTGCTGTGTGTCAGTCACTTTCCGAGGCGGGACCTGCTGAAA
CTAGGCTAAGTCAAAAGGGAACAGACCAAGGTCCATCTGGTCAAAGCCAT
GGTTTTTCCAGTAGTCCTGTATGAATGGGAGAGTCGGACCATCAAGAAGG
CTGAGCACCGAAGAATCGATGCTTTAGAACTGGGGAGCTGGAGAAGACTC
TTGAGAGGAGTCCCTTGGACTGCAAGGAGATCCAACCAGTCCATCCTAAG
GGAGATCAGTCCTGGGTGTTCATTGGAAGGACTGATGGTGAAGCTGAAGC
TCCAATCCTTTGGCCACATGATGTGAAGAGCTGACTCACTGGAAAAGACC
CTGATGCTGGAAAGATTAAAGGCAGGAGGAGAAGGGGACGACAGAGGAAG
AGATGGTTGGATGGCATCACCGACTCAATGAACGTGGGTTTGAGTAAACT
CTGGGAGATGATGAAGAACAGGGAAGCCTGGCGTGCTGCAGTCCATGGGG
TTGCAGAGAGTCACACACAACTTGGTGAATGAACAGCAACAGGAACAGAC
ATGAGCCCTCACTTTCTGACACACAGCACCCAGCGCAGAGAGAAAGGGCT
GTGATTTACAGATGACTCCCAAGCTCACAGTCACGGAGGCACCATGGGCA
CTGTGGCATGTGAGCAGCAATGCCTTTCCCCAGCATAATCCCAAATGAAC
ATCCGCATGTCGAAATCGCACAGGGAATCATCAATGGCTACACAAATTTC
CAGTCACCTGTGAAGCTAAACACCAATGCCCAACTCACAGCAGGAAGTCA
ACGTGTGCGCTCAATTCAACTCACTCAAAGCTCATGAATACTCAAAGCCC
TGAATACTGAATGCAGACTCAATCAAGACATGGATGAGCAACAGACCTTC
TCTCTCCCCTCTGACATTGAAACTATGACTCAACTGCATTTTAAAAATAC
TGACTGATGGTGCAGACACTTTGGAAAAGCTGGGATGATTCTTCCAAACG
TTAAACAGAGTTACCATATGACTGAGCGATTCTCCTCTCAGGTATATACC
CAAGAGAAAATGAAGGTACACATCCAGATAAAATTTATACACGAATGTTC
ACGGCAGCGCTCTTCTGAACAACTCAGTAAAAGTAAACACAACTCAAATG
TCCATCAGCTGATGCATGGATAAACGGCCTACAGTGATCCATCCATACAA
ATGGAATATTCTTTGGCCATTAAAAGACTCATGCCACAACCTGAACACCT
GATGTCACAGACTCAATTAACCTTTTCTAATTTTTCATTTTTTAAAAAAA
ACCAAACTACCCCTTCCAGATAATTAAGTAAAATATTGAAATTCTAATAC
AGCTGGCCCTTGAACAAAATGGAGTTTGGGGAGAAAATCCCCTTATACCT
ACAGGGGGCCCTCCATATCCATGGTTCCTCATCTGTGGATTCACTCACGG
ACTGTGCACAGCGGTTAGGTATTTATTGGGGGGGGGGGGGGATTAACGTA
TAAGTGGGCCGGTGCAGTTCAAACCCATGGGCCAACGGTACATATTTACC
TTGCTACATGTTTGGTCTTAAACAAAAATACCACATAAGGTTGTTCCTGT
GGTTAAATTTGGGCCTATCATCTTACTTCTGATTGCTTCTTTTTAAAGCA
GTACAGCAAAAAATCTTCCGCAAATTTCCATCTTTTATGTTTTTAAAATA
ACAAAATTTCTATCAGTGTTTTTTTTTTTCATTAAAACGAGGAAAGAACC
AGAAAACACACAAAAATAAAGGATAAAATCAAACTCTAAAATTCCATCTT
CCAAAATATTAACAGTGCTTCTTCTTGATAGAGGGTGGAGAGGGGAACCA
TCAGCTTTAGGTTGAGACGTGGGCTTCAACTCTGGTTTTTCATTTTTTGG
GGCGTCACAAGGCTTTCGGGATCATAGTTTCCTGACCAGGGATGGAACCC
ACGCCTCCTGTAATGGAAAGTGCAGGGTCCTAACCACTGGAACCACAAGG
AACTCCCTGGGTTTCCATTCTTGATGTTTGTGCATCTATGATTGTTTGGT
TTCTTCGTCTGGGTATACCTTATCATCTCACTAGATCTATCGATAGTAAG
CTACATGTTTTTCTAATAACTTTCAAGGTTAAGTAGGAAATCAATTTGCA
GATAGACACAAATCATATTAAGAAACTAACAAAGCAAAGAATTAAGATAC
AGGAGCCAAATAAGATACAGCCTCATGCCCTCAATTATAATCAATTTTGC
TGCTTAATTTTAATTCATAATGGTGATGCAAGGGATTGATTTTAGAAGCT
GTCATCTGCTCCAGAAAAGTTTAAAATAGCTGGTTCCGTATTCAGCTTCA
TGTTCAGTACAGTACAGTCGTATCCAACTCTTTGCGACCCCATAGACTAC
AGCACGCCAGGCCTCCCTGTCCATCACCAACTCCCAGAGCTTGCTCAAAC
TCATGTGCATCGAGTCGGTGAGGCCATCCAACCATCTCATCCTCTGTTGT
CCCCTTCTCCTCCTGCCTTCAATCTTTCCCAGCATCAGAGTCTTTCCCAA
TGAGTCAGTTCTTCAAATCAGGTGGCCAAAGTATTGGACTTTCAGCTTCA
GCATCAGGCCTTCCAATGAGTATTCGGGACTGATTTTCTTTAGGATGGAC
TGGTTGCATCTCCTTGCAGTCCAAGGGACTCTCAAGAGTCTTCTCCACCA
CCACAGTTCCAAAGCATCCATTCTTTGGTGCTCAGCTTTCTTTATGGTTC
AACTCTCACATCCATACATGACTACTAGAAACACCATAGCTTTGACTAGA
CGGACCTTTGTTGGCAAAGTAATGTCTGTACTTTTTAATATGCTGTCTAG
GTTGGTCACAGCTTTTATCCCATGGAGCAAGCCTCTTTTAATTTCATGGC
TGCAGTGACCATCTGCAGTGATTTTGGAGCCCAAGAAAATAAAGTCTCTC
ACTGTTTCCACTGTTTCCCCACCTATTTGCCATGAAGTGATGGGACCGGA
TGCTGTGACCTTCATTTTCTGAATGTTGAGTTTTAAGCCAACTTTTTCAC
TCTCCTCTTTCACTTTCATCAAGAGGCTCTTTAGTTCTTCACTTTCTGCC
ATAAGGGTGGTGTCATCTGTGTATCTAAGGTTATTAATATTTCTCCCAGC
AATCTTGATTCCAGCTTCTGCTTCATCCAGCCTGGCATTTCGCATGATGT
AATCTGCATATAAGTCAAATAAGCAGGGTGACAACATACAGCCTTGAGGT
ACTCGTTCCCAGTTTGGAACCAGTCCATTGTTCCATGTCCGGTTTTAAGT
GTCCGCTTCTTGACCTGTATACAGATTTCTCAGGAGCCAGGTATGGTGGT
CTGGAATTCCCATCTCTTTAAGAATTTCCCAAAATTCTTAGAAGGTAAAA
AAGCCCAGTTCATCTTAAAGAATCAAGGAAGCAGCACATCATTATTTAAT
AATGCTTTGTCTATTACCTTCATGGAGAAGGAAATGGCAACCTACTCCAG
TATTTGTGCCTGGAGAATCCCATGGACAGAGGAGCCTGGTGGGCTACAGT
CCACTGGGTCGCAAAGAGTCAGACATGACTTGGCGATTAAACAACAATTA
CCTTGAACTTCAGCAAGAAGAAAGACCTCAGACATGTATCATTTTAAAAC
AGAAGGTGTTCAGTTTTGGCAAAATGCAGTTCTGTTTGAAACGAATTTCA
GGCGGAGAACAAACATCAAAGGCAGGGGTGTACCTTTAGGGTGACTTCAT
GTGTAGACCTCGGAACCAGTGGGCTCCCCAGCACCCGATCATAGGAACTG
CCAAGTGTCTGCTGGGACCTGCCTGGCGAGAAACAAGGAGATTGAAAAAA
AGGAAAACCACAACAACAATCAAAATGAACCTGAAAATTCTTTGGGAATT
GTTCACCTTTGATCTCTCCTGTTCTTCTTTTCCTTTGTCATCTCAAACAC
GTTTTACTGTTTCATACCCTCTATCTTCAACTTTTACCCAAACTGTGCAG
TTTTATTTGCAAAGTAAAAAGGGAAGTAATTAAAATCAGCCATCAGCATT
ATGGTACATGCAGAGCTTTACAAGGAAAACGTCTCAAAGTAAATAACATT
GGTTAGAGTGCAGACAAGCATTCTGCTATCATTCAATGATAATGAACGCC
CTCTTGAAACATCTTTGTTTTAGGGAGTGTATCACATAAAATCAGTAAAT
TAAACCGTACACACCCTAAAGAGAAGAAAGATGCTTCACAGAAAGTTGAT
AGACAGCGCTTGGGCCAATGTTACCATCATCACGCCATGCAAGTTTGCAG
CCATGCACTCAGTTATGTGCTCATGCAAACAGGGAAACGTTAGAATCGTT
TCTCCCCCCCTTCTTTCGGCTTCCTAAAATAAAGCAAAAGGAAGCACTGA
CTACCAGGTCTCAAGTTTTCCGCTGTTATTCTCCTACATTTCCATTTCAG
AGCAGTTCTGTATTTGCACAGATTTCTAAAGAAGCCAGGGGTATATCTCA
CAAGATAACCCTTCATATCACAAACTTGCTTTTCTCAGTCAACATAAATA
GAAAGCAGTCAACCAGCATCTATTTAAGGATATGTACCTCCTTCCTTACA
AAATGTCTTTGGTTGTGAGTGAGTCCAGGCTCCTACAGGATTTAAATTAT
CCACACATTCAAAAACTGAGGAAATTAAATGACTACCACTTTCTGTAAAT
TCTTGTTCAATAGTCTTAAGTGATATAAAAAGGTGCCAGGTTTAATGTAA
TTCAAATTCAGAAGCAGGGCTATGAATGAAGCTGTTTCTTATTAGTGAAT
TAATCAGAACCTCTTGAAACGTTTTTAAGGAAATTCAAGGAAATTATTCC
CACTGAGCTATGTTTATAGTTACACTGTTCAAAGAAAGGAGATTAAAAAG
ATTCTATAAGCAGGTTCATAGGCTGTTAAGTTTCATTACACTATATTTTT
AAAACATCATAAGGATCTGTAAGATGAAAGTAATAACCAGAACAACTGAA
AAAAACTCTACCACTGAAGGAAAATAAGATTTAAACAAAACTCTGCAGTT
GTTTGAATAGACATTTAACGGCATTAGACTGGAGCAGGGACAAGGGAATA
TGGACTTTTCTTATAAAATAAAAATGCATTTCATTATGAAATAAATTCTG
TAGTAGAGATGAAATAGGACAATACTCCTGAGAGTAACCTGACGCAGAAA
GCAATTGACTATTTTCAGGACTGTGTTGTAAACCCAAAATAGCCTTCTGA
TGAGACTGCTAAATACCGTAAAGTATCAAAATATGAGCAGATGTCACCAC
AATAAGCCCCAAATTGCTTGAAAATTAAAACATTAAAATCCACAATAATT
CAATTAGGCAATTAATATATAAAGAGCTCTTACTACAAGCTGTTCAGGAA
AAAAAAAAAGCTTCAGTATTTAGTATTTGGACCTATTCTTTGTCCAAAAC
AGATGGTAACTCACAAATTGAAGGTTATCTTTGATATAAGTTGTTAAATT
CAATAAGAGTAATTAACATCATCTGTGTGTTAGAAACACACAAAACATGT
GAAATATGACTTTGGTTAATGACATTCTGGTTATGCAACATGACAAATTA
ATTAGTTTACTGCTAAAAATAAGAAGTTAAAGTGAGAAATCTGACCTTCC
AATACCTTGTCATTCGTGATTTTCCCATTACGAACAACCAGAGAGAACCA
CTACCCATAAGCCGTGCTGGCAAGAATTCATGGGACTGAAAAATATTTGT
GGGACATGCAAATGATTTTAACAAATGCAATATATTCCTTAATTCTCAAA
GGTAAACACACAACGAAAGTGACGTGAGTCCCTCAGTCACGTCCGATTCT
TTGTGACCCCACAGACTGCAGCCTGCCAAGCTCCTCTGTCCATGGAATTC
TCCAGGTAAGAAACCTGCAGTGGGTTGCCATTCCCTTCTCCAGGGGATCT
TCCCAACCCAGGGACTGAACCCGGGTCTCCTGCATGGTAGACAGATTCTT
TACCGACTGAGCCAAGTAACCCGTAATAAGAATATGGAAGCATCGTGGCA
CGTCCACAGTGTTAACACAGGTGCTTTCAACCAGGGGTGGTGTGTCCTCT
GCCCCAGGGCACTCTAGGATATTAGGCAATGTCTGGAAACATTTTTCAGT
GTCACAACTGGAGGAGGGACACGACTGGCATAGCATTAAGCATCTACAAC
TCGCAGAACAGTCTTCCTTCCACACAGTAGACAGATCAGGCCCCCAAATG
CTACTAATACCCAGAAACTATATTTTAATGGGACAGACTTTAGTGTTACA
TAGGTTAGACTAGGGAAACAGAGTCTAAGATGGTAGTCAGAAGAAGGGAC
AACTACAAGGACCACACGCAGGGCAGACAAAGGAAAGGAAAAAGCCCATG
AAAAGGAAAACCTGAGGCCTGGAACAAGAACCTCTGGAGGGGGAAGAGGC
CTAACACACCCCTTTCCCTGACTGGCTTGACCATACACCCTAATTGCATT
CCCTTCCCTGATTGGTTGCAGATACAAGCCCTATTTTGCAAAGGGAAATA
GCCAATGGAGCCCGAGGAACCGCCAAGGGGAAGAGAAAAATGTATACAAA
GGGAAACCTAAAAATGACACCAGACCACTCCAACGGGTGTGGGTCCACTC
TCCTGTCTCAAGACTGTCCTGCGTGTGGCTTGCTTAATAAATCCTGCCTG
CTTGAGCTGTGTCGGTCTGTCGTTTTAATTCTTTCCTACGAAGAGACAAG
AATGGAGGGGGAAAAACCTACTTGCCAGTAGACTAAGTAGACACTTTTCT
TCGTGACTTCTGCCTTATTCGTACGGTGCCATATTAGAAGTACACATGGG
CCTTCGATTCAGAAAGGAACACCCACGGGACACTTTAGCATAGACTATCA
AAATCTAAAACGGGTTACCGAGGTCAATCACGGATTCTGTATAGAGAAAA
TGGTTCACTAAGAGACACAAAAGCATCAGATATCCAACTGCAAAATAGTT
CAAGGCATTCAGTGAATTAAGAGCATGGGAATAACAATGAATTCTGATAA
GTGGGGGCTTCCGTTGTAGGGGGCACATTTCCTGGTAAGGGGACTAGGAC
CCCATATGCTTCGAAGCATAGCCAAAAAAAAAAAAAAAAGAATTCTGATA
AAGAAAAAATATTTAAGAGCAATGAAGCAAAATAATAACTATTACAGAGA
GAAAGTCTCTGGAAAAATAGGAATACCAGAACATTCAAGGTTCAGCAGAT
AATCCCATCACTTAATGAGGTACAAATGAAAGCAAGAAGAAACTGCATCG
TTCATTTGAAAGAAGAATATCCATGTTTGCAAAAAAAGAGAAATGGCTTT
AACAAAATTGAAATTTGAGTGGAAGGGCTTGAAAATCAGGTATAAAAGAT
TATAAGAGAGGAAATTTATAGGCTTATCCTTTTATGTAAATACAAGTTTA
GCAAAATGTTGAAAATTTCTGATGCTTTGTAATGAATACTTGGAAGTCCA
TTATTCTCTTCATTTCTTAGTATGTTTGAAATCTGCCAGAGTAAAATGGT
TTTTTTTTTTAATTTACATAAAAAGATCTGTGAACTTATTGATTTGCCCT
TGTTACTTATGAGGCTGTAAGCTACTTTGAAAGCAAGAACAATGCCTTCT
ACCATCTCTTTATACTTCACTGTGGCTAACGGACTACCTTACATTCAGGG
ATAAACAGCTCATCAGGGTCTGTTGAAATGTTAAAAACTAAATGTCTTTT
TTGGGGGGAGCTATGGAAAATGCCATGGATGGAGGAGCCTGATGGGCTGC
AGTCCATGAGGTCGCTAAGAGTCGGACATGACTGAGCAACTTCACTTTCA
CTTTTCACTTTCATGCATTGGAGAAGAAAATGGCAACCCACTCCAGTGTT
CTTGACTGGAGAATCCCAGGGACAGGGGAGCCTGGTGGGCTGCCATCTAC
GGGGTCGCACAGAGTCGGACACGACTGAAGAGACTTAGCTGCAGCAGCAG
CAGATACAGTTAAGTGTCTTCTAGCTCTGAGATTCCATGGTATCAAGTTT
CAATTTATCTGCTTATGCTTAACTCTTAACTCACCAAGATACCAAAATTA
CCTGGCCAGGTGATCAAGTCACATGTTAAGTCTTTCCCAAAGCACAAGTG
AAGAGATTCCAAGTTTGGCTTGTTTTTTGCAATCACAAACCAGCAAATTA
AAGAGAGAAGGAAGCAACCTTTACAACACTGTCAGGGGTCTAACACCCTG
ATTTCAAGCAGTCTATTTTGTCTGAGTTCCCTCCAATAGTCACTTAAACG
TCCATAATTAATGAGAAAGTAGAAAAGAGCCTCCCAGAGGAAAGGGCCAG
GGAACTGGAGCTTTGAGAAAAGACTAACATGTGACTTGATGACTGCTGCA
CTGGAAAACCAAGAGGAAAGCGGAAGTGAACTCACAAGATTTGTTGCACA
AGTAAAGGAAACCGTAAACGAAACAAAAAGAAAACCTACAGGCTGGGTTG
CAATTCAACAGGACTCCGTTTCCAAAACACACGAATAGCTCATACAACTC
AGTAACAGAAAACCAAACAACTCCATCAAAAAAACTGGCAGAAGAGCAAA
GTAGATACTCCTCCAAAGAAGACATACAGATAGCCAATGAGCACCTGAGA
AGATGCTCGATGTTGCTCATCACTGGACAAATTCAAATCAAAACTACAAT
GATGCATCACCTCACACAGGTCCGCATGGCCATCATCAAGAAGTCTACAG
ATAATAGATGCTGGAGAGGGTGTGCAGAAAAGGGAACTCTCGTACGCTGT
TGGCAGAAATGCATTTGGTATGGCCCCTATGAAGAATGGTAAGGAGGCCC
CTTAAAAAAACTCAAAGTACAATTACCATAGGATTCAACAATCCCACTCC
TGGTTATGCCGCTGCTGCTGCTAAGTCGTTTCAGGCGTGTCCAACTCTGT
GCGACCCCACAGATGGCAGCCTGGGTATATATCTAGAGAAAACCCTAATT
CTAAGAGATACATGCACCCACATATTCACAGAAGCACTTTGCACAATAGC
TAGGATGTGGAAGCAGCCTACATGTTCACCGACAGATGAAATGCTAAAGG
TGTGGGATGTTGTGTTTAGTCGATATATTGTATCCGACTCTTCTGTGACC
CTATGGACTGTAGCCCACCAGGCTCCTTTGTCCATGGGATTCTCCAAGCA
AGAATACTTGAGTGCGCTGCCATTTCCTCCTGCAAGGGATCTTTCTGACC
CGGGGATCAAATCCATGTCACCAGCATTTGCAGGTGGTTTCTTTACCAAT
GAGCCACCTGGGAAGCCCCTAAGGTGTGGTGTGTGTGTATATATATATAT
GTGTGTGTGTGTGTGTCTCAGTTTTATCTAACTCTTTGTAACCCCGTACA
CTGGGGTAGCCTGCCAGGCTCCTCTGTCCATGGGATTCTCCAGGCAAGAA
TACGATAGTGGGTTGCCATGCCCTTCTCCAATATATCTATATCCATATCC
ATCTACACATATATATACACATATACATATATATAATGGAATATTACTCA
GACATAAAAAAGGATGAAATAATACTATTTTCAATAACATGAATGGACCT
AGAGGTTATTGTATCAAGTCAGACAAAGGCAAACTCATCACTTATACGTG
GAACCTAAAATATGACACAAACGAGCTTAGTTACAAACCAGAAAGAGACT
CATAGACATAGAAAACAGATTTATGGTTACCAAGGGAGATTGGGGAGGGG
ATAAGCTAGGACTTTGGGACTAGCAGACACATGCTACTATACATAAATCA
GATAAAAAACAAGGTCTTTCCGTGAAGTACAGGGAACTACAGTTAATATC
ATATAATAAGCTATAATGAAAAAGAAACTAACCATAAAAACGGTTTATAC
ATAGCTGCATCTACTGTGGATCTGTTCTAAGTGCCTTGAAGTTATGTAAT
CCTGCTAATAAATAATGACAGAATCCTTCCTACCAGAATGTCACGTTTCC
AACGAGGATCCCAGGTACCCCCACGAGCAGTGTCAGACTAGGGGTTCACA
CACAGCAAGTCATGACTTTCTTGTCATGATGCCTTCCTGGCTCCAAGCTG
CAGGCAGCAGAAATTTTGTGAATTGCTGTCCTTCTCTAGGGGCTTCCCTG
GTGGATCAGCTGGTAAAAAATCCACCTGCGATGCGGGAGACCTGGGTTCC
ATTCCTGGGTTGGGAAGACCCCCTGGAGAAGGGAACGGCTACCCACTTCA
GTATTCTGCCCTGGAGAATTGCACAGACTGTATAGTCCATGGGGTTGCAA
AGAGTCGGACATAACTGATTGACTTTCACTTTCACTTTCCTTCTCTAGCT
TGTCTGTGTACCTAGGTATTCACCTAGAAAATGACACTCCATGCTCAGGA
TATAACTTTCCCAAACTTTAACTTTTCTGACACATGCTGAGATGTTCCTG
ACGACTTCCTGACAGATTTTGAATACACCAGTTGCTAGACATTCTAATAA
AGAGGTTCTCCTGCTGGAAGACTATAGATTCCCCAGAAGAAATATGAAAC
CAACGTGCATTTCTCTAGACTCTGGAAGGCTGCAAGCTAAAGATGGCAGC
GCTTGACAAAAATTAATGCAAGTCTGCCAAGATGGTAATGACTTACAAAA
ATAAACTTGACTTTTAACATCATAATAGTGGCAAGAAGTAGGGCAGTTCT
ACACGCGCCTTTACCCACTGGGAATTAAAATCAGGCCATGATCCTCACGT
CTCTTTTCACTCACCCAACTAGGAACACGGGTACCGTGAGGTGAGAGGTC
ATGGTGCATTAAGCGGTTCCTCATCTCCTACCATCACATTCCTCTTTGAA
CACCATTCATTTATGGGATGCTCTTTATTTAGATGGATTAGTTATTTTTT
GTCACTATACTAAAAGGGTACCCAATTCTCTTTATACGTAAACTGGCAGA
CAAGAGTAAAGGGATCTGTTCTCTGTTAAGAGTTCTATTTATGACACACA
CACAAAGATTCTGGGTTGTTTTGTTCATGATAAACACTAATTACGTGCAT
GAATTTCAAGGGGAAGAAAAGGGAAGGAAGGAAGAGGAAGGAAGGATTGG
GAAGGAAGGGGAAGGAATTGGAAGGAAGGAAGGGGGAGGAAGGGGAAGGA
ATTGGAAGGAAGGAAGGGGAAGGAAGGGGAAGGAAGGAAGGAAGGGGAAG
GAAGGGGAAGGGAGGAAGAGGAAGGAAGGGGAAGGGAGGAAGGGGAAGGA
AGGGGAAGGAATAGGAAGGAAGGGGNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
AGGGAGAAGGGAAGAAGGGTTGAAGGATGACGGGGGAGGAAGGCATGAAT
GTGGGATGAACTGGACGAATGGTAGAAGAAAGGGAAGGAAATGAATGAAG
GAAGGGGATGAAGAAAGGGGAAGGGAGAGGAAGGAAGGGAAGGAGGGGAA
GGAAGGTAGTTGAATGTAGGGAAGGAAGAGGAAGGAAGGGGAGGAAGGAA
GGGCATGGAAGTGGAGGAAGGAACAGAAGAATTGAAGGAAGGAAGGGGAA
GGAAAGGGAAGGAGGGTGAGGAGGAAGGGGAAGGAAGGTAGAGGAAGGAT
GGGGATGGGAGGAAAGGGAAGGAAGAGGAAGGAAGGGGAAGGAAGGGAGA
GGAAGGAAGGGGAAGGAAGAGGAAGGAAGGGGAAGGGAGAGGAAGGAAGG
GGAAGGAAGAGGAAGGAAGGGGAAGGAAGAGCAAGGAAGGGGATGGATGG
GGGAGGAAGGAAGGGCATGGAGGCGGGAGGAAGGAACAGGAAGGAATTGG
AAGGAAGGAAGGGGAAGGAAAGGGGATGAAGGAAGGGGAAGGAAGGGAAC
AAGGGGGAGGAAGGGCGAAGCTTGGTGTTTTGAATCCTCGATTAAAAGGC
CCAGGCATTTCAACAAAGGCACCCTAGTACACACGAGTGCATCTCACACG
CAGAGAGAGCAGGAGTAACGCTGCAGGGTCCTTCACAGGGAACACACTTG
TATGTCTCTATTATTTCTCTAACTAGGCTACTCTGCTGACAATCCTCCCC
TTTTCTGAAACCAAGTTTAAAAATCGGGAGATAGTCAGGATATGACGACC
GAGGAGGGAAAAGTTCTAGACTGGTAAAGCAGGGGTGAGCACACTTTCTC
TGCAGGAATCTTGGATCCTTGACACCCAAACTGAGCCTGCTACAACTGCT
CACGTCTGCTTTGGCTCTAAAACAGCTGCAGACGGTACATAAATGGGTGT
TGGATGTGCTTCAACACAATTAAAAAAAAAATAGATTGCAGGTGGGGTTG
GCCTATAGGCAGGAGTCTGCCGATCCCTGCCTGAGGGCAGTATCAGTGAA
AGTCATTCAGTCGTCTCTGACTCTTTGCGACCCCATGGACTACACAGTCC
ATGAGATTCTCCAGGCCAGAATACTGGAGTGGGTAGCCTTTCCCTTCTCC
AGAGGATCTTCCCAAACCAGGGATCGAACCCAGGTCTCCCACATTGCAGG
CGAATTCTTTACCAGCTGAGTCACAAGGGAAGCCCAAGAATACTGGAGTG
GATAGCCTTTCCCTTCTCCAGCGGATCTTCCCGACCCAGGAATCGAACCA
GGGTCTCCTGAATTGCAGGCAGATTCTTCACCCACTGAGCTGTCAGGGAA
GCCCAAGGCAATATCATGCTCACTTTATTAAGGTGTGTACTCCAAACTTC
TTGTGTGCATGTGCGCAGAGTCACTTCACTTGGGTCTGACTCTCTGCGAC
CCCATGGACTGTAGCCCGCCAGGCTCCTCCATCCATGGGATTCTCCAGGC
AAGAACACTGCACTGGGTTGCCATGCCCTCCTCCAGGGGATCTTCCCGAC
CTGGGGATTGAACCCACGTCTCTTATGTCTCCTGCACTGGCAGATGGGTT
CTTTATGTCCCCTGGGAAGATGACCCATATTACATTAAAACAGACAACCT
TCTAATATCCTCAGGGTGACTTCAGACAATTAACATATGACAGAAGTGGT
CTATTCGAGAAGTAAAACAAGTCTCTGCCTTGTTTCTGGGAATGACCACA
GTGGAGGGGGTGCAGTTGGAGTGAGGGGTGTATTTCAGCCCAGGTTCTTG
GTCTCTGGGACACACACACACACACCCATACACACACACACATGTACACC
TGCCATGGGAGCTTTCATGACACTAGCTCAAATGCCATTCTAGGTTAAGA
TGATGCTCTCAGAACCCAAGTGCAAAGTCTCATGAAACTTTTCAGTTCTT
TCACGGATCGCATAAGCGTGAAACTGAACAGAATAAATCTCATCAGTGTG
GAGATGACATCTCTTTTTTTCCCCCTCCCTCGTTAAACACGATTTTTAAC
AGGTTTCGTTTTTAGACTAGTTTTAGATTTCCAGCCAAAGTGACAGGAAG
GTACACAGATTTCCTGTATGCCATTTGCCCCTACACAGGCTCAATTTCCC
TCCTTATCTAGGTCCCCCACCAGAGGGTCCATCTGTTACAACTGATGAAC
CCACACTGACATTATCACCTGAAGCCCATAGTTTATATCAGGCTGTGGCA
CCTTCTATGGGTTTTGACAAAAGACATAAACACACCTGAGCCCCCCAGGT
GGCGCTCGTTGTAAAGAACCTTCGCACTTCAATGCAGGAGACGTAAGAGA
CACAGGTTCGATCCCTGGGCCAGGAAGATCCCCCGGAGAAGGGCATGGCA
ACCCACTGCAGTGTTCTTGCATGGAGAAGCCCATGGACAGGGAGCCTGTT
GGGCTATAGTCCATGGGGTCGCAAGGAGTCGGGCAGGACTGAACGACTTT
CACTCACTCACTCAAGAAAACCGAGGCTTAGCTGACTGACGGGGTTGCAA
GAAGTCGGACATGACTGAAGGGACTTAGTACGCACACACGCATAAAGACA
CGTGTCCACCACTAAACCACCATCAAGAATACTTCCACTGTCCTAAAAGT
TCTCCGTGCTCCAGGGGCTCATCTCAGCACTGAAGATGGTGCTGCAAAGA
GTCAGACACAACTAAGTGATTGAACTGAACTAATATTCCACTGTCTAGAC
GCAACATTCAAATATTTATCCACTCACATATAAAAGGACATCATGGTTTC
TCCCAAGTTTTGGCAATGATGAATCAAAGCTCTATAAACATACGCGTGCA
GGTTTTTTCCTTTGAGTACATTTTTTTAAAAATGCCATTAAAATGATATA
AGTAAGGACTTGCCTGGTGGTCCAGTGGCTAAGTCTCCACGCTCACACTG
CAGGGGCCTGGGCTCGATCCCCGGTCAAGGAATTAGATCCAACATGTGCA
ACTAACGATTCTGGATGATGCAATGAAGCCTAAAGACCCACACAGTTTTG
CACTAAGACCTGAGCAGCCAAATAAATCAACATACTTTTTAAAGAATTAT
CTAATTAACAGATGTTCAGGGGAAAGCTTTTAAGAGGTTCTCAGTTGTGG
GCAACTGTGTCCCCGTAGGGTCATCAGTCAATGTCCGGAGATATTCCTCA
GTGTCACAACTCCGGGGAGGGTCTCTGGCATGCAAGGGGTAGAGGTCGGA
CAAGACATTCCCACGTGAGAAAGGATTCTTGGGCCCCCAACGTAACTAGT
GCTGAGGCTGAGAAAACTCTGCTGCTGCTGTTCAGCCACTCAGTCGTGTC
AGACTCTTTGCGACCCTGTGGACTGTAGCCTGCCAGGCTCCTCTGTCCGT
GGGATTCTCCAGGCAAGAATACTGGAGTGGGTTGCCACGCCTTCCTCCAG
GGGATCTTACTGAGCTGGGGATCGAACCCATGTCTCTTACGTCTCCTGCA
TTGCCAGGCGGGAAGCGCCACTAGCGCCACCAGGGAAGCCCTGAGAAGCC
CTGGTATGAGGATAAAATGCTAACTCTCCCTAGGTCCACTCCGTCTCACC
TCGCAGAGACTGCCCCTATTTCCCAGCTCCCCCTACAGTCCTCCAAGTGC
CCACCGTGTGGTACTCTTCCTTTTAACACAAACGTGATCTGCTAACTATC
ATCCCTGAATCTGAGTCATGAGAGGTGCGTGTCACTGGTTTTCATCACGA
AAACCTTTCAACAGCAAGGCTGCTCAGTACCTGCCTACATAAAATAACTC
GTCGCCCACCTGTTCTCGTAGAAAGTGTACATTTTATCAAGTCCCTCTAG
GTCCATGATATGACTTAACCGTCACAACAATCCTTTTGTTATTCCCACTG
CCTAGCTGTAAAAGGCCAAGTTCTAGAGAGTTTAAATGAACGTAACAGTT
CAGACTGGTTTCACAGCTAGGAAATGTTCAAATTTGTTTGTTGTTGCTCA
GTTGCTCAGTTGTGTCCGATTCTTTGCGACCCCATGGGCTGCAGCACTCC
AGGCCTCTCTGTTCATCATCAACTCCCGGAGTTTACTCAAACTCATGTCC
ATTGAGTTAGTGATGCCTTCCAACCATCTCATCCTCTGTTGCCCCCTCCT
CCTCTTGCCCTCAATCCTTCCCAGCATCAGGATGTTTTCAAATGAGTTGG
CTCTTTGCATCAGGTGGCCAAATTACTGGAGCTTCAGCATCACTCCTTCC
AATGAATACTCAGAGATGATTTCCTTTAGGATGGACTGGTTGGATCTCCT
TGCAGTACAAGGGACTCTCAAGAGTCTTCTCCAACACCACAGTTCAAAAG
CAGCCATTCTTTGGCGCTCAGCTTTATGGTCTAAATCTCACATCCATACA
TGACTACTGGAAAAATCATAGCTTTGACTAGATGGACCTTTGTTGGCAAA
GTGATGTTTTTGCTTTTTAATGTTCTGTCTAGGTTGGTCATAGCTTTTCT
TCCAATGAGCAAGCGTCTTTTCATTTCATGGCTGCAGTCATCATCTGCAG
TGATTTTGGAGCCCAAGAAAATAAAGTCTGTCACTGTTTCCATTGTTTCT
CCATCTATTTGCCATGCAGTGATAGGACCGGAAGCCATGACCTTAGTTTT
TTGAATGTTGATTTTTAAGCCAGCTTTTCCACTGTCCTCTTTCACTTTCA
TCAAGGGGCTCTTTAGTTCCTCTTCACTTGCTGCCATAAGGGTGGTGTCA
TCTGCATATCTGAGGTTATTGATATTTCTCCCGGCAATCTTTGAGTCCAG
CTTGTGCTTCGCCCAGCCCAGCGTTTCTCATGATGTACTCTGCATGTAAG
TTAAATATGCAGGGTGACAATTCATAGCCTTGATGTGTTCCTCTCCCAAT
TTGGAACCAGTCCGTTGTTTCAAGTTCTGTTCAACTTGTCCAGTTCTTGA
CCTGCATACAGATTTCTCAGGAGGCAGGTAAGGTGGTCTGGTATTCCGAT
CACTTTAAGAATTTTCCATAGTTTGTTGTGATCCACACAGTCAAAGGCTT
CAGTGTGGTCAATGAAACAAAAGTAGACGTTTTCCTGGAATTATCTTGCT
TTTTCTATTATCCAACAGATATTGGCAATTTGATCTCACACTCCTGAGCT
GATCTGAAATGTATTCAGTCATTTGCACTGTGTATATGTGTGTTAGTCGC
TTAGTTGTTTCCGACTCTTTGCGACCCCGTGGACTGTAGCTCACCAGGCT
CCTCTACCCATGGGATTCTCCAGGCAAGAATACTGGAGTGGGTAGCCATT
CCCTTCTCCAGGGAATCTAGACGACTCAGGGATCGAACCGGGGTCTCCTG
TACCGCAGGCAGATTCTTCACAGCCTGAGTCACCAGGGAAGCCCTCGTTT
GCACTTTATCTACAAGCAACTACGCTCTATTCAGTTTTGGATTTTCTGTG
ATTTTTTTTTTTTTTTCCCTTCACCGGACCGCCTTCCAGGTTTCCGCAGT
TAGCCTTGACATCTAGAAGATGAAAGCCAGAACGGGAGTGAGTCATCCTG
GGGCTTTGCATTCCATTCACGTGCGCATCTCTCCCATGGCGTCAAACTCA
GATCAACAGCGAATGTCTTATTTATAGCGATGGTTTATTTATAGATGAGC
AGGATGACCAAAAGCACCCTTATGCCTCCAAGAAAGCAGCAGCGTGTGCA
GCTGGACCAGGAAACCAAGTGCGGGAAAAGGCAGTTCCTTCCTGATTCAT
GGAATGCAGTTCTTTGGAGATAAAACACACCCTATTTGTCATTAAAATTC
TTTGTTTCTGAGTGCGTCTGCCTTCAAGATGTTAACGCTTCTCTTGTTCC
ATCATTGGTACGGGAGACTTGTGAAATCACAAGCAACCATTATTATTAGT
GTCATGACTGGGGACTTGTCTGGGAGTTTAGTGGTTAAGACATGGCACTT
TCACAGCAAGGGGCATGAGTCTGAGCCCTAACTGGGGGACTAAGATACTA
AATGCCTCGAGGTGAAGCCGAAATACACACAGACATATATATATATACAC
GTATGTACACGTGTATTCATACACATACATATATATACACATGTACGCAC
ACATTGATGACTGTAAAATCATAGCTAAATATGTACATCAGGCCTCATTT
CATGCAACTACAGCTATTGAATATGAATTTTCCAAACTAGAAAAGGAGAG
CTGGGAATTCCGTGGTCTCTGTGCTTTCAATGTCAAGGGTCTGGGTTTGA
TTGCTGGTCACGTAATTAAGATCCCATAAGCCCTTGTGGTGCAGTGAAAC
AAAAAGAAAAGAAAAATGTCACCCACCATTTTTTTAAAGTCTTTTTTGAT
TTTGTTACAGTATGGCTTCTGCTTTATGTTTTGGGTTTTTAGGCCTGTGG
GGTCTTAAGCTTCCCCGACCAGGGATCGAATGCACACCTCCTATACTGGA
AGGCAAAGTCTCAATCACTGGATCTCCAGGGAAGTCCCGGCCCACCTTTT
TTAGAATTCTGATTCCTCACTGCCCTTTCCTCCTACCTGACGGCTGCTAA
CCAAAACAGACTCACGTGTGAGCGCCAGTCAGTCAGTCCTCCAAAGCCAG
GGACGAACAGGACATGTGGGTTGGTCTGTCTCGGCTAAGATGACTAAAAC
AGTTTGAGAGTTTGTTTCTGCCTAGCTTTCAAGACACAAGCTTGCCTTCA
GGTGCAGGGGCTGCAGGAGGCAGTTTCTAGTACGGGCTCCAATATCACTG
GGGGAGGCGACTGCAGTCATGAAATTAAAAGACACTTGTTCCTTGGGAGA
AAAGCTACGACCAACCCAGACAGTGGCTTCAGAAGCAGAGACATCACTTT
GTTGACAAAGGTCCGTCTAGTCAAAGCTATGGTTTTTCCAGTAGTCATGT
ATGGATGTGAGAGTTGGACCATAAAGAAGGCTGAGTGCCAAAGAATGGAT
GCTTTTGAACTGTGGTGTTGAAGAAGACTCTTGAGAGTCCCTTGTACTGC
AAGGAGATCCAACCCGTCCATCCTAAAGGAAATCGGTCCTGAATATTCAT
AAGAAAAACTGATGCTGAAGCTCGGATACTCTGTCCACCTGATATGAAAA
AACACCCTGATACTGGGAAAGACTGAAGGTGGGAGGAGAAGGGGACGACA
GAGGATGAGATGGACCGATGGCATCACCGACTCAACGGACATGAGTTTGA
ATAAACTCCAGGAATTGGTGATGGACAGGAAGGCCTGTTGTGCTGCACTC
CATGGGGTCGCAAACAGTTGGACATGACCTAGCGACTCAACGGACAACAG
CAAGTATGTGATTATTTGGAGAAACAAACACTTGAGACAATTCGTTTGAA
GCAAGACCAAGAAATCTATCAGTTAAGTAAAGCCTCCTCTTTTAAAGGAA
TCTGACATAAATTCTGCGAGGCAGAAACCACCTCTGTAAGCTGATGGTGT
GAGGTACCAAAAAGATAACACACCTCATGGAAGGAGGAGGCACTGCAGGG
CCTGGCATTACAAATGTCAAAAAAGACTTGTGGTTTCCTGTAGTTTCAGA
GGGCGACTGACTCTTGAGACAGAAAAAGATGATACACAATTTAACGCCAG
AAAAAAATAACGCACAATTTAATGGTGCTCAGAATTCCCAGATTCATCCC
AGGGTGTGATGGTCCCCACTGGCTTTCCAGGGTGGGCTTGGGACGCGGAA
ATCACATAAAATAGAATGTCACCCCGCATGATCCGGGATCAAAGGTGCAC
GTGTAGGACAGAGCTACAGGACGTGTGCGTCAGGCTCACATCTGCATGGA
CGTGGTCCCCGCAGTGATTTCCTCATCGCCCTCTGCCATCTCACAGACTT
GGGAGAGCCCATCGCTGGGCAGTCAGAACCTGGAAGTCATCCCAGGTGGT
CTGCGTTATATATCCCATCTCCCTTCCACTGCACCACTGGCCTATCTTTA
GACGCACTTTTCAGGTACTAAAACAATAAAATAAACACATTGGCACACAC
TGATTCTTCTAGCCCTGTATATTTGCCTTGAAGGCCAAACTCTTGCCTCT
GTCTTGGGTGGCTAGTCTCAATCACGCCATTCTTTACACCCAATCTAAGC
TGGCTTCCTTTTCTCCAGATTCCAGCACGCTGTTATTTGCCAGAAATTAC
ACTCAGTGCTTGGAAAGGGTAGTGGTTCCCTTCCGACACAAGCTGGCAGG
CTCTCGGAGAAGCAGTGTGGTCCTGGCGTGACCTCGGCTAAACTCCAGAC
TCCTTCGCTCGGGGCTGTGTGTCCCCTGGTGAGGAACTTAGCCTCTCTGG
GCCAGTACAGGAGCACGGCGTGTTTAGCAGTTATGCTGCAGAAATTCACT
GATTCAGACACATAAAGCCGGACGCGTAGGCCTGTACAGAACGACAGTTA
ACTGCAACATGCTAATGGCTGTGGAGTGGTTACTGGAATGATGCTGTGTT
CCTTCTGTGCTGTTCATCATTTGTTTCAATTTTTAAAGCAAAAATGAAAA
CCAAGATGATTAAATATATTAATATATAAAAATTATATTATAAATAACAA
TGAATAAATAATAAATGATATAATACACAAATCTATACAAATACATAATA
CAAATATTAATATATAATTGTATGTATTTAACATATAGTTAAATATATTA
ATATATAATTAAATATAATAATATATTTAATCATCTTGGGTTTTATTATA
TATATACACACAGACACACACACATATATAAACCTAAAAGCCAATATAAA
TCTACATTTTCACCAGTGACTCCCACAAAGGAATAAATGTCAGAAGCTTC
TTTAACAGGATTAAAACAGTCACGTAAGTGAAAGTATAAGAGAATGATGT
TTTTGTTTCCACTGGAACATAATCACAGTTATCTCTGGTTTTTTCTTAAA
CGGAAACTAACTGCTTGGTTTTCAAACAACTGCACTTTTAAGTGGGCAAA
TGTGTCTGAGAAAGGAAAAGAACAGGGTCAGAAGAAGGAAAGAAACCAAT
CAGGTTAGACAGAGCGTAGATGCTCCAGTTCTTGACTCTAGCAGTATCTT
CATGCTACACCATCAAATGATTTTATCCGCCTAGTACTCTTTTACTGATC
CCCTTTTACCAGTCTAGTAAGCAACAGGCTTCGTGCAAACATGATTTCAA
GTGTAGTGTTAGCGTGTTAGCCGCTCAGTCGTGTCCAACTCTTTGCGACA
CCATGGACTGGAGCCTGCCAGGCTCCTCTGTCCATGGCGTTCTCTAGGCA
AGGATACTGGAGTGTGTGGCCATTCCCTTCTCCAGGGGATCTTCCAGACC
CAGGGATGGAACTTGGGTCTCCTGTTTTGCAGGCCGATTCTCCACTGTCT
GAGCCACCAGGGCAGCCTAATGATTGCAAACACAGTAAAATGCAAATGCT
CTATGTCCTATAAAAATCAAGTATAAAGGAAAACTAGGATTTAAGTGAAT
ATCCTAGTTTAAGCTTAATCTGATTTCCTTCGATCCTGTCCAAATTTCCC
TGGGGTCCTAAGATCACCCTCATGAATTATGCAATGTGTTAAATCCCTAA
GTAAGTATACTGGAGTGTGTAGTATAGCTTCCCAGGTGGCGCTAGCGGTA
AAGAACCCACCTGCTGATGAAGGAGACATAAGAGGCGGAAGATCCCTTGG
CGGAGGAAGCGACAACCCCCTCCAGTAGTCTTGCCTGGGAAATCCCATGG
ACGGAGGAGCCTGGTGGGCAACAGTCCGTGGGGTCGCAAAGAGTCGGACA
TGACTGAAGTGACTTAGCACGTCTGTACTACAGTTTGGAAGGTATATCAA
GATGTTCTGGGGATGCGATTTCTGACAACACAGTAAACAGAACAAAGTGT
GACCCCGAAATGGTACTGGGAAGATGCCATTGGTAACAGGGAACTGGTGC
TATGTCAGATTACCTGGTTGTGCAAATAGAACTATTCACAAGTCACACAC
TGCCCTAAACACACAGAAAACGTCTCTGCCAAAAAAATGCTGAAACGCCC
TGCAGTTTCAAGTATGACATTTACATGGTTTTATAGAAATACCACAGATT
AAAAATAAAAGACAGTGTCTTTTGGTAGGAGAGAAACCTTGTTCCTCCAA
AGAGTATATGAGAAAACTAAAGTGAACTTTGAGATATCTTTCTCAATTTG
GTTCCTCCATTATACAGAGATTTACTTATTCAAATAAGTAGAGAAGAGAC
AGCCCCTTACAAAGAAAAGGATGCAGGGTCTGGAAGTGCGAGTAGAAAAG
TTAATGTGTCTTAAACGGGTGACTCTCCAACTTGACTGGGGACTTTATAC
TGTGCTCTCCTGCTGGCAAAGGTCAGTACAGTCAAAGCTATGGCTCTTTA
GTAGTTACGTACAGATGTGAGAGTCAGACCATAAAGAACACTGAGTGTCT
GAGAATTGATGCTTTTGAACTGCGGTGTTGGAGAAGACTCCTGAGACTCA
CTTGGACTTAAAGGAGATCAAACCAGTCAATGCTAAACCCTGCATATTCA
TTGGAAGGACTGATGCAGAAGCTGAAACTCCAATCCTTTGGCCACCTGAT
GTGAAGAACTGACTCACTGGAAAAGACCCTGATGCTGGGAAAGATGGAAG
GTGGGAGGAGGGGACGACAGAGGATGAGATGGTTGGATGGCGTCACCAAC
TCAATGGACATGAGTTTGAGCAAGCTCTGGGAGTTGGTGATGGACAGGGA
GGCCTGGCGTGCTGCAGTCCATGGGGTCGCAAAGAGTCGGATATAACTGA
TCTGATCAACAACAAATCTCCTACTGAAGGAAGTCTCCTGGGGGCTCCCC
CCCTCCCCATAAAGAATAAAAATCTCTATCTGGGACCTTAAGAACTGCAG
TCCAGGAGACACAGATACAGGTAAAACCAAAGGAAGCGTTCGGGGAAAAG
AAAGAACCAGGGGCTCGTAAAGACAGAATTCACAAGGGTGTTACAGTTGC
CCAGTGAGAATTATGAGTGACTCGGATGCAAGTCACAGGTTATTTGTCCT
CACAGAACCATGAGTTATTTTAGGGTTCCCAGGGGCTTTTTTTTGAGACT
TAAATGTGAAACAGTGACAGCAAAAGGGTTCAGAACAGCGTGATTAATTA
AGGAGACATTTGATGCACCCACTGACACCCCGGAAAAGCCCCTGTACACA
GGCCAAGCACTCGACAAGTGTGAATACGCTCAGCTGACTTCTGGGGTCTT
AACCTGTTCGATTCGATTAGCGCTTGGTAGATACGTTCTTAAAGGGCCAG
AGACTAAATACTCTTCATTCCATGAGCCGCAGAGAGCTACAACCGTGCAA
TACAGGCATCAGCGGGTTTAGCTGTGTTCCAAGGAAACTTAATTACGTAT
AATGGAACTGAGAGAATTTTCACGCGTTATAACTTGTCATTCTTCTTGGC
TTCATTTTTCAAAAAACACTTGCTTTATTTATTTTTAAATTATTTATTTG
GCTGTGACTAGTTGCGGTATGCGAACTCTTAGCAGCATGTGGGATCTAGT
CCCCTCAGCAGGACTGAACCGAGGGCCCCTGCATTGGGAGACTGGAGTCT
TAGCCGCTGGACCACCAGGGAAGTCCCTGGTTTCTACATTTTTAAGTGGT
CCCCCCCCCCGCAAAAAAAATCAAAAAATGGTGGTTCAGTGGTTAAGACT
CCATGCTCCCAAGGCAGGGGTCCCAGGTTCAATCCCTGGTCAAGTAAACT
AGATCCTACGTACCTCAAGTAATGATTCCCTCTGCTGAAACTGAGACCCA
GAGCAGCTAAATGAATGAATGAATAAATAAGTAAGTTTAAGTTCAGTGCA
GTCGCTCAGTTGTGTCTGACTCTTTGCGACCCCATGGGCTGCAGCACGCC
AGGCCTCCCTGTCCATCACCCACTCCCAGAGTTTACCCAAACCCATGTCC
ATTGAGTCAGTGATGCCATCCAACCATCTCATCCTCTGTCATCTCCTTCT
CCTCCCGCCCTCAATCTTTCCCAGCATCAGGGTCTTTTCCAATGAGTCAG
TTCTTCGCATCAGGTGGCCAAGGTATTGGACTTTCAGCTTCACCGTCAGT
CCTTTCAATGAACACTCAGTACTGATTTCCTTTAGGAAGGACTGGTTGGA
TCTCCTTGCTGTTCAAGAGACCCTCAAGAGTCTTCTCCAACACCATAGTT
CAAAAGCATCCGTTCTTTGGTGCTCAGCTTTCTTTATAGTCCAGTTCTCA
CATCCATACATGACTACTGGAAAAACCACAGCCTTGACTAGATGGACCTT
TGCTGGCAAAGTAATGTCTCTGCTTTTTAATATGCTATCTAGGTAAGTAA
GTACTGGGTTGGCAAAAAAAAAAAGAAAATGTAGAAACCATCCTTAGCAT
ATTTAATGTACAAAAGTAGACAAAGGGTTAAATTTGGCCTGCAGGCTAGC
TCTGCTAAAGCTGTGTTAGATAAGATGAGGCATGGTTAGATTCCATTGCT
ATCACAGCTTGCAAAGATTACTGAAAAACCAACCTCGGAAAGCATTATGA
CAATTCGTCAAAACATTCAACACACAGTTGCCATATGATTCAGCAATTCC
ACTTTTAGGTATAAAATCGAAAGAACAGAAAGCAGGAACCTGAACAGATA
CTTGCACCCCAATGTTCTAAGTGACAACATTCACAATTGCCAAATAACAG
AAACAACCCACATGTCCACTGATGGATGAATGGATTAAAAAAGTGTTGTT
TATTCATACAGTGCAATGCTATTCAGCTTAAAAACAGAATGAAACTCTGA
GACATCCTACAACATGGGCAAACCTCGAAGACGTCGTTTAGTCGCTAAGT
TGTGTCCGACTCTGCGAGACGCCATGGACTGTATCGTAAAGGAAATCAGT
CCAGAATACACATTGGAAGGACCGATGATGAAGCTGAAACTCCAATACTT
TGGCCACCTGATGCGAAGAACTGACTTAATGGAAAAGACCCTGACGTTGG
CAAAGACTGAAGGCAGGAGGAGAAGGGGACAACAGAGGATGAGATGGTTG
GATGGCATCACAAACTCCCGGAGTTGGTGATGGACAGGGAGGCCTGGTGT
GCGGCAGTCCATGGGGTCGCAGAGAGCTGGACACGGCTGAGCGACTGAAC
TGAAGTAATGCACTGCAGCCCACCAGGCTCCAATGTCCACGGAATTTTCC
AGTCAAGAGTACTGGGAGTGGGGTGTCATTACCTTGTCCATCTGAAGACA
CTATGCTAATTGAAATACGTGAGACACAAAAGGAGAAATAATGTATGATC
CCACTTATGGGAAGTATCTAGAGTAGTTAAAAATAGAGATAGAAAGTAGA
ATGGTCTTGCCAGGAGAGGGGCGAATGGAGAGCAGAGTTAAGTTTAAGAG
TTTAAATCTGGAAAGGAGGAAAAGTTCTGCAGGTGGATGGTGAATGTACT
TAATGGTACACAGCACAGGAATACTGTGTACTTACAAGTGGTTGAAAAGG
GGAACTTTTATGTAATGTATATACTTTTCCATAACAAAAGAGATCTCAAA
GGAAATGAACCTTCAGTGCAAATGTACTTCTTCCAAAGACCCAGCGGCTA
GTTACAAACTTAGATCAAAATGTTTGTGACAGCTAAAATGTGCATTCGCC
AGTCTACTCTGAAGAAAAGTCAAGAATATCTGCTTTTTCCATAAGCCAGA
GCCAAATCCTTCACTGTGAAAAGAAGCTGTCCTCGGTTATCTTCCAAGTC
ACAGATGACATTTTAAGATACTTGCCACTGCAGCGGAGGCATTCCCTAGA
AAACAGTCTCAAAATCTGGGTAATTTCTGGAATTTCTGAACGTTGGTCAA
ACCTAGAAATTCTTAAGCCAGCTTCTGTCCTTGGAGAATTTATCTTCATG
GAAGCAGAACGACACATTTTGGCTAATTGAATTTGTATTCATCTTTGTGG
TTAGGAAATGCGATTGATGTTATTGCTAAATCAAGGTTTGTCATAAAAGT
CATTCAAAAAAACAAACATACGATGCTGTTACGTGATTCTCAGGACCATA
CTTGTGGTTGTTAGTAAGAACTCTAGAATCCAGGAAAAAAACAGCAGCTA
ATCAATCTAGGTGGGCCGTTCCATCCTACCTAAGATGAAACCTGAAAAGT
GAGGGGGAAAATTGGGGGTGGGGGTGGCAGATCTAAGCATAATTCTGGGT
GGAAGCTATTTCCCTTATTTAAAAGAAAAGAGTTAATAAGAATAAAATGA
GATTAATGCATGTTCTGTCAAAAACTCTCCCGATGGCTGGTTCACTAAAG
TCTGATCTCCTGCAAACTTGGGGCTTCCCAGGTGGCTCAGTGGTAAAGAT
CTGCCTGCCAATGCAGGAGACATGGGTTCGATCCCTGGGTTGGGAAGATC
CCTGGAGAAGGGAAAGGCTACCCAGTCCAGTATTCTGACCTGGAGAATTG
CATGGACTGTATGGTCCATGGGGTTGCAAGAGTCGGACATGACTCAGTGA
CTTTCACTTTTTTGMTGAATGAAAATACACAAGGGGTTATCAAATGACTT
TGGAAAACTTTCAGGCACCTTAGGTTTCCAAAAGGAATTTTTGTTTTTTA
AATTGGCCTCACCTCTYGGCTTGCAGGATCTTAGCTCCCCGAGCAGGTAT
GGAACTCAGGGCCCAGGGAGTGAGCACGTGTAGTCCCAACCACTGGACTG
CCAGAGAATCCCCAAAGGCCTGTTTCTGTCATTTGGTTTTCTCCCAGTGC
GTGCATGCTAAGGCGCTTCAGTTGTGTCCGACTCTGTGCGACCCCATGAA
CTGTAAGCCCACCAGGCTTCTCCGTCCCTGGGATTCTCCAGGCAAGAATA
CTGGACTTGGGTTGCCGTGCCTTCCTCCAGGGGATCTTCCTGACCCAGGG
ATCGAACCCACATCTCTTCCATCTCCTGCACTGCCAGGCAGGTTCTTTTA
CCACTAGCGCCACCTGGGAAGCCTGGCTTTCTCCCTCACTCGACTGCAAA
TCGACCACCTTTTACGACAGGACTATGCTGCGTCCAGCCGCCCTGTGAAC
ACACGCAGCGTCCCTGCCCGCTTCAAATCCCAGATCCACATCTCAGGTCC
CAGGCCTCGCCGCTGCACGCTCAGCCCAGGACTGGCCCCCATGACGCGTG
CACGTAGCGGACCCTGAGGCATCTCGAACATGGCGTGTCCACGTGCGTCC
AGCCTGCCAGCCCCAACCACCCACACCAACACACCCCCCCACTCCAGCCC
ACCTTGAGAGTTAAAAATGACCCCTGCCTCCCACCCAGGGGGACACAGGC
CAGACCCGTAGTTCCTCCAACCAGGATATCCACACATCCGCTGGGGCTCC
TGGCCCAAGACTGTCCGAGTCTGCGCTTTTCCTTCTCAGATCCGCTGGGC
TGTGCTAGTGGGAAATAAGACTGCCTTCATCCTGGGACCCACTAGGACCA
CTCCAGTTCTGACCCGCAGCAGCAGCCAGCCACCAGTCACAGCGGTGGGA
AAAGTCTGCAGGCTCTCAACTTGAAATGCAGCACCGTGACCATCGTATCC
CGCAAGGAGCCGTCGGGTGGGCCCCGCACACAGCTCTCAACCTCATCTCC
TGGTCGGTCTCGGGCCCCCACCATGCTGGCCTGGCCTCCTTTTTGTCCCT
GGGGTTTCCAGAGCTCAGCGTCACAGGCTGCCGCTTTGGAGGTGCTGTTC
CCTTTCTCTGGAACTATCTTTTCCCCGCTCTTCCCAGAAACATGCTTCAG
ACGACGGCTCAGCAGTTTCCCCTTCTAAAAGCTCCCATGTAGAAGCCCTT
CTGGTCCGAGGCCTTCAGCTCCACGGCCCCACTCCTTCTTGACCACATGC
GTCACCATCAAAGCATCCTGTGCCATCTGGGTACTGCCTGTCTCTCAGGT
GGCAGGCAGGCTCCGGCAGGACATGGCCTCTGGACCACCGTATCGCTGAG
CTCAGAGTTCTCCCTCCAGCCACAAAACTGAGTTTCGTGCCAGATGCGAC
AAACACAGGTGATCAACAGCCTACGACATACGAGCACGACCTTAAAAGTC
ACAATACAACACAAAAGGGACATCCCGGGCGTCCAGTGGTTAAGACTTCA
ACTTCCAAAGCAGGGGGTAATGGGTTCGATCTCTGGTCAGGGACTTAAGA
TCCCATATGCCTTTGCCGGGGAGGGGCAAAAAACAAGAACATGAAGCAGA
ACCAGGATGGGAACAAATTCAGTAAGGACTTTAAGAAATGGTCCACATAT
GAAAAATCAAAAAGAGAAATTAAGGACACAATCCCCTTTATCGTCGCAAC
AGAAAGAATAAGATCCCTTGGAATAAAGCTACCCAAAGAGAGAAAAGACT
TGCATGCATAACACTATAAGACCCTGATAGAAGAAATCAAAGATGACGCA
AACATGGAGAGATATTCCATGTTCTTGGATTGGAAGAATCAATACTGTGA
AAGTAGCTATGCTCCCCCAAAGGAAGCTACCAGATCCAGTGCTATCCCTA
TCAGTAACCAATGGTATTTTCCACAGGAGGAGAACAAAAAATTTCACAAT
GTGTATGGAATTCAAAAGACCTCGAATAACCAAAGCAATCTTGAGAAAGA
AAAAGGGAGCTAGAGAAATCAACCTTCCAAACTTCAGACTCTACAGTCTG
AAAATAGATAGCTTCGGTCATCAAGATTGTATGGTACTGGCACAAAAACA
AAACGACAGAATGCCCAGAGATAAACCAAAGCACCTATGGGCACCCTATC
TTTGACAAAGGAGCCAAGAATACAGAAGGGAGAAAAGACAGCCTCTTCAA
GAGTTGGTGCTGGGAAAACTGGACAGCTACATGGGAAAGAATGCAACCAG
AACACTTCCTAACACCAAACACAAAAGTAAACTCAAAATGGATTAAAGAC
CTAAATGTAAGACCAGAAACTGTAAAACTCTTAGAGGAAAACATAGGCAG
AACACTCGATGATATAAATCATAGCAAGATCCTCTATGACGCACCCTCTA
GAGTAATGGAAATAAAAGCAAAAATAAACAAATGGGACCTGATCAAACTT
AAAAGCTTTTGTACAGCAAAGTAAACAATCAACAAGGTGAAAAGTGAGCC
CTCAGAAGGGGAGAAAATGATAGCAAATGAAACAACTGACAAAGGATTAA
TATTCAAAATATACAAGCAGCTCTTGCGGCTCAATACCAGAAAAACAAAC
AAACCAATCAAAAAGTGGGCAGAAGACCCACGTATCTCAAAAAAAGAAAT
ACAGGTGGCTAATAAACGCATGAAAAGATGCACAACACGACTCATCATTA
GAGAAATGCAAGTCAAAACTACAATGAGATACGACCGGACGCTGGTCACA
ATGGCCACCATCAAAAAACCTACAAACGATAAATGCTGGAGGCAGCGTGG
GGAAAAGGGGACCCTCTCGCACTGCTGGTGGAAATGCAAACTGATACAGT
CACTATGGACAACGGTGTGGAGAATCCTTAAAAACAGAGGAATAAAACTA
CCATCTGACCCAACAATCCCACCACTGGGCATATAACCTGAGAAAACTGG
AATGAAAGAGACACACGTACCCCAGTGTCCACTGCAGCACTGTCCACAAA
AGCCAGGACGTGGACGCAACCTAGATGCCCATCGGCAGATGAATGGATAA
AGAAGCTGTGGTACATATACACAACGGAATATCACCCAGCTATGAAAAAG
AACACATTCGAGTCAGTTCTAACGAGGTGGATGAACCTGGAGCCTATTAT
ACAGAGTGAAGTGAGAAAGAGACAAACACTCCGTATTAACGCATGTATAC
GGAATCCAGAAAGATAGGGCTGATGAACCTATTTGCAGGGCAGCAACGGA
AACGCAGATGCAGAGAACAGACTTGTCGGCACCGGGGAAGGGGAAGGAGC
GAAAATCGGAGAGAGTAGCATTGAAACATATACAGTACTGCATGTAGAAT
TAAAAGCGGCAGTGGGAATTTGCTGTATGACGCAGGGAGGTCAAATCCAC
CTAGATAGGGTGTGAGGTGAGAGGTACGTTCGACAGGGAAGGCCTCACAC
ATAACCTGTGGCTCACTCATGCTGGTGTCTTGCAAAAAACCAACACAATA
TTGCAAAGTGATTATCCTCCAATTAAAAATAATTTTTTAAAAAAGAGAGA
GAGGGGGGAAAAAAAGGCCCATGTTAAAAAAAACAAAGAAAAACCAAATA
CGATTACTTAAGACCAAACTCCAGTGAAGATGGCACTTTCCTCCTTCATG
ACTCACGTGTGGCTTTCAGGTCGCGCTGGGCTTTTATACTAAGCGCTAAC
GACAAGCAGAAAGTGTGTGTGGAAGTTGCCTGGAATCATATCCTTCTAAA
TGCATAGTTGAACGAGCAGATGGCGTGACACCTCCACATCACCTACATAC
AAGGGTATATCCTCGGCATTACGTGAGACAGACAATCTGGCAAGTCCTGC
GCGAACACGCACGCACACACATATGTAATTACCAGCGTCCGCTCTTCGCT
GGCCGGAGAAGGCAATGGCACCCCACTCTTGCCTGGAAAATCCCATGGAC
GGAGGAGCCTGGAAGGCTGCAGTCCATGGGGTTGCCGAGGGTCGGACAGG
ACTGAGCAACTTCACTTTCATGCACTGGAGAAGGAAATGGCAACCCACTC
CAGTGTTCTTGCTTGGAGAATCCCAGGGAAGGGGGGAGCCTGGCAGGCTG
CCATCTATGGGGTCACACAGAGTCGGACACGACTGAAGCGACTTAGCAGC
AGCAGCAGCAGCTCTTCGCTGGCAGGGCTAACAGATAAAAAGATGAATGG
CAAAAGGATCTCGTAAAGGATTCAGCCACTCCCAGGGGCTGAAGCTGCTG
CCACCAGCCAGGTACAGATTTCCACGAGCACAGCTCAGGAAAGGGACGCC
AGCCAGTCCAGGCTGTTGACAGGTCCTTACCCGTGCTCTGTATCTCCTCT
ACAGAAGAACCTTAAAGCGGGATGCCCGACGCACAGGGATGGGTGAACAC
AGCTGGCAAGGTAGTGTTCACAACCATCCGCGCGTCTTTCCTCCCGGTAC
CTCTGGATATGCCCGCTCCCAAAGCCGGTCCGGCTTTGCTCAGCTGCACC
TGCTGCTGTAATGACACGCGTTCCTGAGATATTTCCCTTGAGAAAATATG
AGTGCATACAGCAGCCGCTGACCGGATTTTTGCTAAAGCAGAGTTGATGC
TCTGCAGACAGGGTAAATGGCAATCATTTTAACAGAGGCCGTGGAATCAC
ATGTGGGAAAGACAACCACGAAGGGCTTCCCTGAGGGCTCAGCTGGTAAA
GAATCTGCCTGCAAGGCAGGAGACCCCGGTTCAGTCCCTGGGTTGGGAAG
ATCCCCTGGAGAAGGGAAAAGCTACGCACTCAAGTATTCCTGGGCTTCCC
TGGTGGCTCAGCTGGTAAAGAATCTGCCCACAATGGGAGACCTCGGTTCG
ATCCCCAGGTTGGGAAAATCCCCTGGAGAAGGGCAAGGCTACCCACTCCG
GTATTCTGGCCTGGAGAATTCCATGGACTGTATAGTCCATGGGGTTGCAA
AGAGTTGGACACGACTGAGCGGCTTTCACTTTCAGGATAACCAAGACTGA
AACGCAAGAAGACCTTACGGTCCTTGCCCCTCACGTCCCCCACCTCCTTT
TTTGTCTGTGGAAAAACGTTAGCCAAAGAATAAGTTTAATCAGAGGAGTG
AGAAAATGCAGAAACATAGGAAAACAGTCAAAGGAGACTAACTATAAACT
ATTAACAATTTAGCCACTAAGAATTGATGGCTTCGAATTGTGGTACAGGA
GAAGGGTTTTGACAGTCCCTTGAACAGACAGCAAAATAAAAGCAGTCAAT
CCTAAGAGAAATCAACCCTGAATATTCCTTGGAACGAATCGAGCTCCAAT
ATTTTGGCCACCTGATGACAGGAGCCGACTCATTTGAAAAGACCCTGATG
GTGGCAAAGATTGAAGGCGGCAGGAGAAGGGGACGACAGAGGATGAGATG
GTTGGATGGCATCACCAACTTGACGGACATGAGTTTGAGTGAGCTCCGGG
AGTTGGTGATGGACAGGGAGGCCTGGCGTGCTGCAGTCCGTGGGGTCACA
AAGAGTCAGACGTGACTTAGCGACTGAACTGAACTGAGCTGAATGTGCAC
AAATCAAAGAACTCATTAGTGCTTCCAAATGCCAGGAGTTTATGACAGCT
AACAGTGATTACACACACTTTCTGGAGACCTGAGAAAGCCCAGAGCTAAA
TGGCTATAACTTTATAATATGAAATTAATGCAGCAAAATCACCACAATAT
CCCAGAGACCGCTAGTATTCTGAAGCCCCTTCATGAGATAGTAGTATCTG
AATGCTGACTCTATAACACAGATAAATTAAGAATATACACAACTAGCTTC
TTGGGCTCCCCTGGTGGCTCAGATGGTTAAAGAATCTGCCTGCAGTGTGG
GAGACCTGGGTTTGATCCCTAGGTCTGTAAGATCCCAGAGAAAAGGGAAT
GGCAACCCACTCCAGTATTCTTGCCTGGAGAATTCCATGGACAGAGGAGC
CTGGTGGGGTACAGTCCATGGGGTCAGCAAAGAGTCAGACACAATGGAGC
AACTAACCCTTTCACTTTTTCACAACTGGTTTTTTAACATTAGAAAGGAT
ACGCAATGGAATTGGAACTAAATTCATATTGAAATGAGTACATTCTGCAC
TTTGTTGAATGCATTAGCTCATATAAAATCATTTATGACAGTTTTTTTAA
AAGCAGCTATCTGTGAAGTGAGATATATAACCATGAAATACGATAAATTT
TTTAAAAAAGGATACATCCTGCTGATCCTTTTAATCTAATCCCTCACCGT
ATCTCCACTTCTGCCTTCACTGGCGTAAATTTATCTCCCCAGGAGGTGTT
AAATCAAAGCCTTCTGGGCCTCAAAAGGAATGGTTCGTTGGTTGCATGCT
TGGGCTCCAGGGTCCGCTTGTGGGCATCGCCATGAAAGAACCTACTTCAT
CATGTTAATCTACGCCTGTGTTGCCACTGCTGCAAATCATTTCATCAGCA
CTATCTTCGCAGATTCCATATATGTGTGTTGCGTGTGCTTAGTCACTCAG
TCACTTTGCGACCCCATGGATTAGACAGTCCGTGGAATGCTCCAGGTCAG
AATACTGGAGTGGATAGCCTTTCCCTCCTCCAGGGGATCTTCCCAACCCA
GGGATCGAACCCAGGTCTCCCGCATTGCAGGCAGATTCTTTACCAGCTAA
GCCACTAGGGAAGCCCCAAGAATCCTGGAGTGGGTAGCCTATCCCTTCTC
CAGGAGGTCTTCCAGTTCCAGGAACTGAACCGGGGTCTCCCACATTGCAG
GCAGATTCTTTACTAGCTGGGCCACCAGGGAAGCCCCAAGAATCCTGGAG
TGGGTAGCCTATCCCTTCCTCAGGTCTTCCAGACCCAGGAATTGAACTGG
GGTCTCCCACATTGCAGGCAGATTTTTTACCAGCTGAGCTACCAGGGAAG
CCCAAGAATCCTGGAGGGGATAGCCTATCCCTTCTCCAGGAGGTCTTCCA
GACCTAGGAATTGAACCGGGGTCTCCCGCATTGCAGGTGGATTCTTTACT
AAGTGAACCACCCGGGAAGCCATTAATATACAATATTTGTTTTCCTCCTT
CTGACTTACTTCACCCTGCATTTTGACTTGTCACCTTGAAAGTGTATGAT
TTATTATAAGATTATGTCTGTTCAAATATACTTATAAAAGGTTTTTCATT
TTAAATTATCAGTTGTGTTTGACATAGGGATTTGAATTTATCCTCATGAT
TTAAGTCCCTATGATGTAACTAATCCAACTCTAATTCACTCAGTATATAT
AGAATGACTTTGCAACTACTATTCAAATTAAAGTACTAGCCAATCTAACA
TAGCCTCCTTGTAAAATATATTTACGATAAAAAAAAGCTGCAAGGGATTA
ACCTTAAAACATTTTTAAAAAAGAACTTACCTTAGTACCCCTCTGATGGT
TTCTTTCATTTCCGAAGCCCCCCCAAAGTGCAAGAGCTAAGATCCTCCCA
TCTGTCTGCACTGACCTACCTACTGTGGTAGTTCTCACTGGGGGCGATGT
TGCCAAGCACTCAGGGTGGGGGGACATCCGACAACATGTAGAGACATGTT
TGGTTGCTTCAACTGTTAGGAGAGAGTTCCTGTACACTACTGCATAGAAA
CCCAGGATGCTGTTCCACACCCCGCAATGCAGAGAACAGTTCCTACCATC
AAAAACTGGTGTCACTGTTCAGTTGCCCAGTCGTGTCCAAGAGCCGGACC
GTAAACAAGGCAGAGTGCCAAAGAACTGATGCCTTCGAACTGTGGTGCTG
GAGAAGACTCCTGAGAGTCTGCCCCCAAAACCCAGCTCAGTTCAGTTGCT
CAGCCGTGTCCGACTCTGCAACTCCATGGACCACAGCACGCCAGGATTCC
TTGTCCATCAACAACTCCCAGAGTCTACTCAAACTCATGTCCACTGAGTC
GGTAATGCCATCCAACCATCTCATCCTCTGTCGTCCCCTTCTTCTCCTGC
CTTCAATCTTTCCCAGCATCAGTGTCTTCCAGTGAGTCAGTTCTTCACAT
GAGGTGGCCAGAGGATTGGAGTTTCAGCTTCAGCATCAGTCCTTTCAACA
ACACCCAGGACTGATCTCCTTTAGGATGGACTGTGTTGGATCTCCTTGCA
GTCCAAGGGACTCTCAACAGTCTTCTCCAACTCCAAATGTCAAAAGCACT
AAGGTTCGGAAACCCATTTTATCCCCTTATGTATGCGGGGAATTTATCTC
TTCTTCCTGGAGCCAGTTTTCACAGTCTATGTAAGTTGTACCTTTTTTTC
CCCACCCTGTTGTTCCTAAATTGTCCATGAGACACTTTCATTTTTGTAAG
AACTCAGTCTTGCTTCTGTCTTTCCAGCATTCAGACTCTTCACGTAATGG
ACAGCTGCTGGGAATAAGTCGTCATGAACTGACAATCCAGACACCTTTGA
CACCTCTTATTATTTTTTTTTTATATTTACTTTTAKTCTTGCTTATTTGC
TTGCACCAAGTCTCAGGGCACATGGGGTCTTTTGTTGCAGCTCACGAGAC
CTTGTGCAGTGGCACCTGAATTCAGTGGTGGCATGTGGTACCTATGAACC
CGGGTTTCCCTGCATTGGGTGCAGTGACTCTCAGCCACTGGACCACCAGG
GAGATCAACATACCTCTCTTTGGAAGCTACTATCTCACAAAGATAACTCT
CCTCCAGTCCCCACAGGTTAATTCTCAACGGGAAACTTCTACCTTCTTCC
CAAACTTAACAGACTTAACAACACTGGAGATAACACCACTCAACTTGACG
AATTCTAAGTCAGATAATCCCACCATCAACACACCATCTTTCACTCCAAA
CATTTAGAGCATCAGCTTCATCGAGATGGGGAAAACAAAAACAAAACAGC
AGGCCAAAAGTGAAGGATGCTCTGAAGCTTCTAGAAGGCARCAAAGCTAA
CCTTCTGCTCCAGGGAGAAGACATTCTGTATGTGGTCCTGTTTTGACCAG
AGAACCCAGGCCTATGATCAGGGCAAGGCTTTGTGGAAAGGACGGAGGGG
GCAGGCGAGAAACGGAAGACCACGGTGATCATTCGGACATCAACAGGCTA
AGAATTCACAAGGCATCTATTTCAAATGCCGCAGACCAGCCGTCATGAAC
GCTGTTTGATCAGTGTCTACCACGAAAAGCTGTACCTCCCAGACATCACA
AATCCAGCCTATTCAGTCCCAGGTCGGTCAAATAATAATAATAACAAAAA
ACACGGTGTCTTGTAGGTGTCTGTGCTGTCTCCACCCCCACATCGCATTA
TTCCTACAGGATTTTAAAGGCTCAATGAGTCAAAATGGGGTCACACAGAT
GAAGTCCACATTTAAGCACTCATGATAAATGGGTCTGCTCCTCCGTCCAC
ACCAGGGAGGTAAAAGTCCGCCCTAAATGCAAGGAAGTCCATCATCACAC
ATGGGGGCCTCACACAGCTAGGTCTCTCTGCATTTATCTCTTCTTAATTT
CTACAGGGCACAAGCCTCCATTCAACAATACGATTTTCACGGAACGGAAA
ATTCACGGTGTCAGGTCAAGAGGAGCGTTGAGAACCGATACGCTGGATAA
AAATAATTATAATGATCTACCAAGCGCCCTGGAAAGCAAAACAGCATGAC
AGGCCCAGTAATACATAAACGTGTTCCATCTGCATACATTGTAATAACAC
GGCCACTGGAAGAAAATCCAGTGTGAACTTTCCTTACTGCCTCAGCGTCT
GTGATTTAAACAAAATCAGGGGCTTCTTATTAATCAGTTCAATTCTCCTC
TGAACCACCATCCACTCCATTCAGCCTGCACAACCAAAAATTTCTCATTA
GCTTAAAAGAGACCGTGCTCAGGGAAAATCATTGTGTGTGCTAATTTCTA
CCAAAAAAAAAAAAAAAAAACCAGGAGAAATCAATTTTTCCTTACAAAGT
TTCTAACTGGCAAAGGTCCACTCAAGAGTGCTGTGGTTTTCTGCTCCCCC
CACCAAGTACTATTATATTAATATAAAGGGACATAATAGTTTACATATTT
GTTGAAAACACAGAAAGAAGTTAAATGAACTTTCCTTTTCTATTTACATG
GCTGAAAAATTATGAACAAATAATCCAAACACACCATATTCACATTTAAA
AATATTCAATGAATACTCAACATTTCTACTCTATTTCTTTTCACTGGGTG
GGGATATGTGTATGATTGTTAGAATCTGCTCTCTAGTAAGCACCCCACTC
CAGTACTCTTGCCTGGAAAATCCCATGGACGGAGGAGCCTGCTAGGCTGT
AGTCCATGAGGTCGCATAGAGTCGGACACGACTGAGCGACTTCACTTTCA
CTTTTCACGCACTGGAGAAGGAAATGGCAACCCACTGCAGTGTTCTTGCC
TGGAGAATCCCAGGGACGGCGGAGCCTGGTGGGCTGCCCTCTCTGGGGTC
GCACAGAGTCAGACACGACTGAAGTGACTCAGCAGCAGCAGCAGCAGCAG
GAGTATTCTTGCCTGAGAAATCACAGACAGAGGAGCCTGGTGGGCTACAA
TCCATGGGGTCACAAAAAAGAGTCAGACACGACTAAATAACAACACCCAA
TGTGTCATACCCAACACATACTTTTATCCACCAGGATGTACGCGTTCTGT
CTAATTTGTTCACTGTTAGATTCTCTGCATCTAGAAGAATTCCTGAGACA
GGTGGCAAGTGCTCTAGAAACATTTATTTACTGGTTAGGTGAACGCGTTC
AGTCTGAGAGATGGTGTGAACGCAAATGACCCAGGAGCATACAAGTTCAT
CAAGAACTTCATGTGCAAATATTCTGCAAAGTGCTACGTGGTGTGGAAGG
ACCGTAGAGACAGACGCCTAAGCCCCTTCCTGTACAAGAAGCCTGTGCTA
CAAACCACACAGGCTTTGCATAAAAAGATAACTCCCAACAAACAGCACAT
TAGACTAAGTCTGACCGAGGTAGGAGGAAGACAACAAAGGCATGCTCTTT
CATCTTTATTACTTCATGCAACCACTATAAACTCCCCGGAGATCATATTC
CCACTTGGTGCACAAAGAAAGAGGTTCCTGGTCACCTGGCTGAAACTCAG
AGTAAGTGGTGGGCAAGAAAGGAAACACAGATGAGCAGAAGATGGAACCC
AGAAGCTCTCTGCAGGAGCCGTTGCTGTACCATATACCCCCATCCTCGTC
CCACAGTCTAGAWTTCTGCKTKRAACACGTGATTGTTTTAGCAACACCGT
GTTCCCACTAGAGTGAGGACAAGGTCGAAGGTAGAATCTGCTAGACTACC
ATTATCAGACTACCATTATCCAATTCCATCCCTCACCCTTTGAGAACAGG
AGACCTGGATTCTGTGTGGGCACATGGTCACACCCCTCTCAAAAGGACAA
GTTTTCCCCGTAACCCAGTTCTAGCCAATGGCTACAAGCAAAAGTCAAAT
ATGAAATCTCTAGTCTCTGCTCTTAAAGGCAATGAAATCCCCCTCCTCAC
TCCTACTCAGGGATGTGATTGTGAGGGCAGGAGCTGGGGCAGCCATTCCA
GACACAGAGGAGGACAGAACTTTGGGGCTCCTACTGTGAGGCAGTGCTTT
ATGGAACGAGAGGTCCATCACCCCACTTGCAAAGCTACAGAAAGTTACAT
CTCAGCTGACTTAAGTGACTCACCCCATCACTGCCAAATTACCTTGCAGA
AAGGTCAGAAGCTGCTTCCTGGCCTCTTATAACAACAGAGACATTCATCA
ACAGGGAAGAGGAAGTGAAGAAATTACAAACTACAGCTACTAATGATTTC
CTGGTCAGAAACCTAAGACTTCACAGTGTGTTTTGGAACCAGTGTTCATT
CATGGGCACCCTAGGGTGTTTTACCATATAAACAAAGCCTGAAGAAAATC
CCTTTCATCAGAGGAGGCATCAGTTAGAGGCAGCCCTGAGTTTATGACTT
TCTGGCTCTGAACCTGTATGAGTTTCAAAGCGTGTGCACATGGGGACCCC
TGAACTTATAGGGTGCCAGGCTCCTCTGTCCGTGGGATTCTCCAAGAATG
CAGGAATACTGGAGCAGGTTGCCAGATCCTTTTCCAAGGATCTTCCCGAG
CCAGGGATCGAACTGGCAACTCCTGCAATGGCAGGTGGGTTCTTTATCAC
TAGCATCACCTGGGAAGCCCAATTCAACCTCTGAGACCTTTTTTTAAGTG
CACAGCGGGAAGAACCAACCACCTTTATCTCACGGGGGATGCCAAGGGGT
CAGAAAAACAGCGTAGACCGAGGACCTCGTTCCCTTCCACGTCAGCACCG
CCTTTTGTGGCCAGGAACCCATACATCCGGCAGAGGTAAGGGGAGGTGGC
CACGCATGAACACGGCTCACAGCGAGGCAAACAAATCCTCATGCTGACTC
ACCTGGGTATACAGAACACTTTAGGAAGGCCGACAGTGAAAGCAGGGCCC
GTGTTCAGACTCGGATGTCACAACCAACCCACCGCCTGTGATCTCAAGCA
CTGCTTCTTTCCTTTGTGGTGTGAGAGCTCTGATGGGAGATGTGGATGGG
CCAACGGAAATGCGACGTGGATGGGCCAACGGAAATGCGACGTGGATGGG
CCAACGGAAATGCGATGTGGATGGGCCAACGGAAATGCAAGGTGAGCTTA
CATCTAATTAAAGAATGTTTAAACGCAAACTAATTTTCACAACATATTTA
AACTTAACCCAGCATATCCCAAATAGCATCATTTCAACTGTAGTTCACAC
TGGTTTTTAAAAAAATTATTGAGATCTTCGACATTCCTTTTACCTACAAA
ATCTCTAAAATTCCCAAAGTCTATGTATTTTCCACTTGCGCCACATCTTA
ATTAGGAAACGGAATTTTCAAAGGAAATAGACCACGGCTATCAGAGTTCA
GAAGATGTATTCCGTACACTGAAAACACAGGTACACACACCCATGTGATT
CGCAACATACAACTTATGGAGCAGCTGAACCAAGGATCGAAATTTAAATC
AATAAGGAAATAAAATCTCTAAATCTGATCCTCCGTAACCCTAGCTATAT
TTGAGGGTGCTCCTTGGGTCTACAAGGCTTGTGGCCCCTAAACTGCTAAG
AAGCCTGCAGGAGTGAAAGTTTGATGATTGTAGTCTGCAGGATGGAAGGA
CCTGGGGACATTCCAGGCCACACGATTCCTCTGTAATCGGTCCACATGCA
AATTCTTAAATTATGCACCCACAGCATTTTTTTTTCCCCTCGACAAATTT
CAAATAGATTAAAAAGAAAACCGGACAGGCCACCCAACTTTTAAATTTTC
TGAAAATAGTGTAACTTCCTGCTAGAAAATTATTTTTCATAATGCACATT
TTCATATTTTTACATAGACACTGGAATTTGGGGGCTCAGACTGGACTGAC
CAAGGCAACGAGCCCAAAGTAAGAAGTGCTGTAGGTTTAAATTCCACGCT
GGATTTTGCAGACTTAGTACAGAAATGGAAAGAACATAAGATATGTCTTG
GATGATTTTTTAAATATGAGTAACTTGTCAACATGACACTATTTTGGGTA
GTGAGTTGATTACATTAATACATATGTTGCAAATAATGGGCTTCCCTGGT
GGCTCAAACGGTTAGATTTGCCTGCAGTGCAAGAGACCTGGGTTTGATCC
CTGGTCCGGAAAGATCCCCTGGAAAAGGGAATGGCAACCCACTCCAGTAT
TCTTGCCTGGACAATCTCATGGACAGAGAAGTCTGGCAGGCTACAGTCCA
CAGTGTCCCAAAGAGTTGGGACATGACTGAGAGACTGTTTTTTTTCATTG
TTGCAAATAATATTAATCGAGCATATATTTTGGAGAAGGAAATAGCAACC
CACTCCAGTATTCTTGCCTGGAGAATTCTTTGGATGGAGGAGCCTGGTGG
GCTGCTGTCCATGGGGTTGCACAGAGTCAGACATGACTGAATCGACTTAG
CACGCGTGCATGCATTGGACAAGGAAATGGCAACCCACTCCAGTATTCTT
GCCTGGAGAATCCCAGGGACAGAGAAGCCTGGTGGACTGCCGTCTATGGG
GTTGCACAGAGTCACACAAGACTGAAGTGACATAGCAGCAGCAGCATATA
TTTAGATATATATTGAAAAAATAATTTATGATTTCTTTTTACTTCTCTAC
TGTGGCTACTAGAAAACCTTAAATTAAATTTATGGTTTGCAATCTACTTC
TATTTGGCAAGTGCAAAAACCCAGACAGTTTCCATGGTGCCTGCAGGCTC
TAAAATGTGTTGCTAATAGTCAGCTAGATGAAACCATAGCATTTTTCCAA
GGCTTCTTTTGTGTCCACTGCAAAATTTTAATTAGATGCCCATCGCATCA
TTTTTTTATTCCGTGACCAATTCAAGACAGATTAATAATCACAAATTGTT
CTAAATGCAGACCACCTTTCTCTAGTAAAAAAAAAAAAAAAACAATTTTC
AGTTTGAGGATTTTTTAATATAGGGACTTATCACAAGTTCTGTCAAGACG
CCTGAAAGAGTTGTGGGTCTCAAAAAAGCCACGGTCATTGGCAGGTTGCT
CGCTTTGTGGGCAAACACCGACCTCTCACCTGGGTGTGTACTGTTACACC
TTCCAGTTTCAGAGCCACCTGGCCCAGCAGACAGCAAGGTGCGCTGTGAA
GTCCAGGGCTCACTGCAGAGCACTGGGTGTGGACCGAGACTGGCTGAGCC
TGTCTCATCTATAACCTGGGGGGTGACGTCACCAGAGCTGCACCCCTCTT
CCTAGCAGGTTGCCATGGGAGACCCAGGGTGATGATGCTACAATGGACTC
TGTACCACAGTCAACAGTACAGCTCAAATTTCAGTGCGACCGTGTTTAAG
ATGGGCTTCCCTGATGGCTCAGTGGGTAAAGAATCCGCCTGCAAACGCAG
GAGACACAGGAGATTCGGGTTCAATTCCCTGGATCGGGACCAGGAAATGG
CAGCCCACTCCAGTATTCTTGCCTGGAGAATCCCATGGGCAGAGGAGCCT
GGCGGGCTACAATCCAAAGGGTTGCAAAGAGTCGGACACGACTGAGCTTG
CACACATTCTGTTTAAGATAGGGTGCTGCTGAACAGATATTTTAGCCAGG
ACTAAGGACAGTTTTTCTTGCCGTTATTGTTATATTAAACCTCCTGTTAG
ACAAGGCTTCCTGCCCCTTACACTGTCGGCTATCACTTTTAAAGTTTAAT
TAAGCCTCGCCTACTCAAAGCGCTGGAGAACCCACAGCTAGAGATGCTTA
GGCTCCCAGCACAGGGAGCTGTGTGCATCTCATCAATGATGACAAACTGA
ACATTCGAAGGCCTCCTTTTGCAAGAGGCCCTTTATTGTCACTTTGAGGT
CTACTTTGAACTAACAATAAAAAAAAAATACACCATTCAGAGATGGATTG
GTTGAGTTGCCTAGCCACTGAGTTGTTTTCTAGCCACTGAGTCTCCTGAG
TCGTGTTCCACTCTTTTGCAACCTCAAAAGACTGTAGCCCACCAGACTCC
TCTGTCCACGGGATTCTCCAGGCAAGAACACTAGAGGGGGTTTCCATTTC
TTTCTCCAGGGGTATCTTCCTGACCCAGAGATCGAAGCCGAGTCTCCTGC
ATTGGCAGCCGAATCTTCTGCATTGGCAGGCCAATCCTTTACTGCTGAGC
CACCAGGCAAGCCCAGGTTGGTGTTAAGGAGAAGGAGTTTATTTTGCAAC
AATGGTACGACAGTATAGGAGAGCTTTCTACTTTTACTACTTGGGCAGAG
GGAATATGGCTTCCGTTTGCCCACAGTTGGAACCGGTACCCTGCACCTAT
CTGTGTTAACCACACGCCCTGGGAAACGGAGTTCAAACTGAGCATCTCTC
ACCTTGAGCCACCGTGGGGAAAGGAACCTGGGAGTGAGTCCTCTCCCCAT
TCCTCATTTCTGTGTGTAATCGCAGAGTGTAGAAACAAGGAAACTCCTAG
TGTGTAGCAGTAACATACTGAGAGGAAACAGGACCCTGAGACCTGAGAAG
ATGCATGTTACTATAGAAATAAGGTCATTTTTCCACGTATTACCTCATCG
ATTGAAATCTGGCAGGAAATGATTCATGGCTAGACTCTCACCTATTGAAC
ATAATCTAGAGCAAAAACATTCACACATCATTGAGAAGCGAAAGCCGTTA
AGCTACAAGGCGACAAGAACCGTATCCCAAAATCATTACATTTGTTTTTA
ATAATCAATGAATTTTTTTTTGTTTTTTGCGGGCACATCAAGAGGCATGC
AGGATTTTATAGTACCCTGACCAGGGATCAAACCAGCAACCCCTGCCGTG
GAAGTGCCGAATCTTAACCACCTGACCGTCAGGGGAGTCCTTCTACTGAT
CATTGCTGTATCATCTTTTTCCTCGCCTTTCATTCATTTCCTTTCCAGGT
TTGGAAATGGAAAAAAAAAAAAAAAAAGAAATACTTAAACACTCTAAAAG
GAGGAACTGAAATAGATCTTTGCAAAGCTTTCCAAATGTGTGATGTTACA
CAACAGCGCAAGGAGAGAGAAGCAACAGCAAACGCAGAGACGCCTCACTG
ATGACTTCAGAGCAACACAGAAAACAACCAATCCCCTCCTTGTCCCTAGC
ATGCCACGAAACGGCACCGTGCTTCAAAGACACGGCAGAGACATTCGATC
CATTCATTTGCACAGCAAAATATATGTCACCCGTACACACTCAGTCACAC
CTAGACACACACCCTTGGGAAACAAACTTGGGCTAATGACAAGTAGAAGC
AAAGTGCAAGCCGGCCTATTGATAATTATAAATATTTTAAGATCTTTTGC
AGGCTAACCTAAGAATCTGCGCTTTTCCTACAGATGTCATTCTGTAAAAA
CATCGTCTTCTGATAAACACACTGGGAATCGTGAGATGAGCTGAACTCAC
GTAGAGCAGTGATATTCAAAGGCATCAATTCTCATCCATGAGCAGTCACA
GCGCACATCCCCCCCTCCCCCCACAACGGCTTTAAGGAAAGGGGTCAAAG
AGTTAATGAGCAAAGAAGGTATTTTGGTATAATCTCAGTAATAAGTTCAT
ATGCTGACCCGCCTTCCTGCTTTCTGGACTCAACTCCTGTTTAAGACGCA
GCCCCAATATACAGACTCACAGTCTGGCTCCAGTCGGGAGACTCAACTTC
CCGGCAAACGCCAGAAACGGCCCGTGTAGCTGAAACTCTGACAGTGCAGT
CTTTATTAAAACAGCTGGCATTCACGTGGAACCCAGAGAGCGTAACCCCA
AACAGAACCCGAGCCATTGCTTGCAAAGCAACTTACGTCCAAGTCACAGA
AAAAAAAAAAAAAAAAAATGCAAAAGACTGGCGAACAGGATGAAATAGTC
CACCTCATTTTTTTTTTAAATCAAAGACATGCAAAAACAAAACAAAAAAA
GTGCCCACGTTCCCGTCTCAGTTGACCACAATGTCCTGAGCTGTCTGAGA
GACGGGGCCCCACCCCCACCCCAGCCCACCCCACCCTGGGCTCCCTGGCA
ACAGGGAACAACCAGCACAAACTTTCAAGGAAAGCAATTTGGTAATCGGT
CTCAAGAGCCCTACAGCTCCATTCCCAGCCATCTCTCCTAAGGCAAAAAA
AAAAAATTGGAAGCAATCTGAAGCTCCGGCAACAAACGTTAAATCAATTA
CGGCACACAAACTCGACGGGATGGATGCCTGAAAAATGTTTTCAATGAAC
GTCGGCAATGATCACAAGGCTCACTCCGTAAGGTTAAGTTGGGAGAGCCG
GGTCACAAAGCTGAACCTTCCTGACGTTTCATGCACAGGAATACAGGCAG
CTGGGAGGAGATGTACCAACAGTCTGATGGGGGAACTGGCGTGGGTGGGC
TCAGAGGTGTTTATTTTGTTTCCAGATTCCATTCTTTGCTTTTTTTCCCA
AATTGCTCATTCTATGCAGCACATTGCTTTTAGAAATCAGAAAAGAGGGA
CTGAAAATCCTGGCCAGCAAAGAGTAATGACTAAATTCCTCATCTGCCAA
GGATGAAGAAGATGAATTAAGACAGCGATGTACCTGTTCACCTCTTTCCC
AAGAACGTCAACGATGGTTTTCCTCAAGAGGCGCGGTGTGCCAGCTCGGG
GCGGGGGCGGGGCGGAGATTCCTTCTCCTCGGGGGTTCACAGTGATCAGC
CTCCTCTGAGTCACCTTGTGAAGGTTTATGATGCCAGAGTCTATAAACAC
AACATTAAATATTTATGGTTGATATTTGCTGCTTCTAAAAATAACGTGGA
GGAGAAGGTCTCTTAACAAGGCCTCCTGTTGACCTTCTCGATGAAATCAC
CAAGGCTGCTGATTCTGTAAACACGATGACAGCCACCCACAGGCCCTGGA
AATCCGTAGGCCAATGCCGCCTTCTTAATCCAGTGGACCCCCCCAGCCCC
GGGGGGCCCTTTCCACCTGAGCCAGGTACCCTTGGCCTTCATACCCCAGT
GTTGATGGGGCCCCAAACCTGCTTATGCGATGGGGCATCTTACTCCATCA
ATGAAATGTAACGAAATATTACATAAGCTTATCAGTTAGGAAGACAGTCT
TTTTTTCCCCCTACCAAAACTACTTTCGATGCTTTGTTACAGACTAACAG
ACACGTCATCAGACAAAAATGTACCATGAGGGGCTTCCGGAAGGTCCGGT
GGTTAAGACTCGAGTGAGTGTCCACCGCAGGGGATGTGGGTTTGACCTCC
TGTCGGGGAAACTCAGATCACACATGCTGTGTGCATGGTACAGAGTGCAG
CCCAAAATTAAAAAAAAAAAAAAAAAGTTATCATGAAAACGCTGGCGGGG
TGGACAAGAGTGAAGAGATCTCAGAGAAGGCAGTGAACATGCAGATGGAT
TCCGTGCGGAGGTGGTTTCCTTCGGGAACCACTGCGCTTCACGGGACTCA
ACCCTGGAAACGGCAGAGGGTGCATCATGGGTACAGTTTATTACGGACAG
ACGACCCCAAACTGGAATTACTGGACCCGTATTCCAAGACAAGGCTGAAT
TACCTTCCATCAAGCAATGAGTGAAGAAACCTGCGTTTAAATTTGCTGCT
GTTTAGATGCTGTCGTATCCCACCCTTTCGCAACCCTGTGGACTATAAAG
CCCGCCAGGCTCCCTCTGTTCACGGGATTTTGCAGGCAAGATCACAGCAG
CGGGGGGCTGCCCTGGCGATCCAGTGGTTCAGACTCCGTGCTTCCCACTC
AGGGGGCACGGGTTCGATCCTTGGCGGGGGAAATAAGATCCCACGAGCCT
CATGGTGCAGTCCCCAAAAGTTAAAAAAAAGAAATACTACTACTGGAGTG
GGTTGCCATTTCCTTCTCCAGGGGATCTTCCCGACCCCGGGATCGAACCA
GCATCTCTTGCCTTCGCAGGCTGTTTCTTTATCGCTGAGCCACTAGGGAA
GCCTAGCATTCAAACAGCTTAAGCTTAAGTCTCCAGGAGGTGACGCTTAC
CTTGCTCCCCCTATAGCAGTAAGCATGGGCTTTGAAAGAAGTGTGGGCTT
TGAAAGAAGTGTGGACACACAAGAGAGGGGAAAGTGGTAACTCGACACTG
AAGGGACTCTGCAAACGCAAACCCTGAGCAGAGGGCAAGCTCTGCAAGCT
CATCAGCATTAGGTCACGTTGAGAGTCCCGGCCCTGGAGGCAACGTGATC
ACAGGGCCCGTCTCTGCGGTCTATGTCCCCCAAACTCATAAGCCCAGCCT
CATCACGGGGCACACACGTGCCAAGCCCAAAGTGAGAGACGTTCTACGAA
AGTCGCGGCCGGTATCCCTCAAAACAGAGCCACCACCACCAAACAAAGTC
CGAGACGCTGCCCTGGCCCAAAGGAAAGATGGAGACAGGACAACTGAGTT
CAGTGTGATGGGGACGGGAATTCTGAGACAAAAAAGGACATGGATCGAGA
AAAGGTTAAACGTGAAAAATAATCAGATATACAGTGGACAAAATTTTTTT
AAATCATGTATACGTGTTTCATTTCTTTTTTTCTAATTGATTTATTTTAA
TTGGAGGCTAATTACTTTACAATGCTGTAGTGGTTTTTGCCATACTTGAC
ATGAATCCACCACGGGTGTACATGTGTCCCCATCCTGAACCCCC

What is claimed is:

1. A transgenic non-human male mammal whose genome comprises a trans-inhibitor of a gene encoding for a protein having biologically activity of myostatin operably linked to muscle-specific regulatory elements and integrated on the Y chromosome; wherein expression of said trans-inhibitor results in said mammal exhibiting muscular hypertrophy.

2. The transgenic non-human male mammal of claim 1 wherein said trans-inhibitor is selected from the group consisting of myostatin latency-associated peptide (LAP), catalytic RNA, siRNA (small interfering RNA), follistatin and dominant-negative actin type II receptors.

3. The transgenic non-human male mammal of claim 1 wherein said muscle-specific regulatory elements are myosin light chain 1F promoter (MLC-1F) and enhancer (MLC-1/3E).

4. A method for producing a transgenic non-human male mammal exhibiting muscular hypertrophy comprising the steps of:
a) providing a somatic cell obtained from a non-human mammal;
b) introducing to said somatic cell a nucleic acid encoding for a trans-inhibitor of a gene encoding for a protein having biologically activity of myostatin operably linked to muscle-specific regulatory elements such that said trans-inhibitor is integrated on the Y chromosome;
c) introducing a nucleus of said somatic cell of step (b) to an enucleated oocyte;
d)cultivating said oocyte of step (c) in vitro to obtain an embryo;
e) inserting said embryo into the uterus of a foster mother non-human mammal and allowing said embryo to develop to term;
f) obtaining a founder male mammal carrying said trans-inhibitor; and
(g) breeding said founder male mammal with a normal female mammal to obtain F1 male offspring exhibiting muscular hypertrophy.

5. The method of claim 4 wherein said somatic cell is a fetal fibroblast.

6. The method of claim 4 wherein said trans-inhibitor is selected from the group consisting of myostatin latency-associated peptide (LAP), catalytic RNA, siRNA (small interfering RNA), follistatin and dominant-negative actin type II receptors.

7. The method of claim 4 wherein said muscle-specific regulatory elements are myosin light chain 1F promoter (MLC-1F) and enhancer (MLC-1/3E).

8. A transgenic male bovine whose genome comprises a trans-inhibitor of a gene encoding for a protein having biologically activity of myostatin operably linked to muscle-specific regulatory elements and integrated on the Y chromosome;
wherein expression of said trans-inhibitor results in said bovine exhibiting muscular hypertrophy.

9. The transgenic male bovine of claim 8 wherein said transinhibitor is selected from the group consisting of myostatin latency-associated peptide (LAP), catalytic RNA, siRNA (small interfering RNA), follistatin and dominant-negative actin type II receptors.

10. The transgenic male bovine of claim 8 wherein said muscle-specific regulatory elements are myosin light chain 1F promoter (MLC-1F) and enhancer (MLC-1/3E).

11. A method for producing a transgenic male bovine exhibiting muscular hypertrophy comprising the steps of:
a) providing a somatic cell obtained from a bovine animal;
b) introducing to said somatic cell a nucleic acid encoding for a trans-inhibitor of a gene encoding for a protein having biologically activity of myostatin operably linked to muscle-specific regulatory elements such that said trans-inhibitor is integrated on the Y chromosome;
c) introducing nucleus of said somatic cell of step (b) to a enucleated oocyte;
d) cultivating said oocyte of step (c) in vitro to obtain an embryo;
e) inserting said embryo into the uterus of a foster mother bovine animal and allowing said embryo to develop to term;
f) obtaining a founder male bovine animal carrying said trans-inhibitor; and
(g) breeding said founder male bovine animal with a normal female bovine animal to obtain F1 male offspring exhibiting muscular hypertrophy.

12. The method of claim 11 wherein said somatic cell is a fetal fibroblast.

13. The method of claim 11 wherein said trans-inhibitor is selected from the group consisting of myostatin latency-associated peptide (LAP), catalytic RNA, siRNA (small interfering RNA), follistatin and dominant-negative actin type II receptors.

14. The method of claim 11 wherein said muscle-specific regulatory elements are myosin light chain 1F promoter (MLC-1F) and enhancer (MLC-1/3E).

15. A fetal fibroblast cell comprising nucleic acid encoding for a trans-inhibitor of a gene encoding for a protein having the biologically activity of myostatin operably linked to muscle-specific regulatory elements such that said trans-inhibitor is integrated on the Y chromosome.

16. The fetal fibroblast cell of claim 15 wherein said trans-inhibitor is selected from the group consisting of myostatin latency-associated peptide (LAP), catalytic RNA, siRNA (small interfering RNA), follistatin and dominant-negative actin type II receptors.

17. The fetal fibroblast cell of claim 15 wherein said muscle-specific regulatory elements are myosin light chain 1F promoter (MLC-1F) and enhancer (MLC-1/3E).
